(19) United States
(12) Patent Application Publication
(10) Pub. No.: US 2011/0045101 A1
(43) Pub. Date: Feb. 24, 2011

(54) FUNGICIDAL SUBSTITUTED AZOLES

(75) Inventors: Thomas Paul Selby, Hockessin, DE (US); James Francis Bereznak, Newtown Square, PA (US); John Joseph Bisaha, Hockessin, DE (US); Amy X. Ding, Wilmington, DE (US); Mary Ann Hanagan, Newark, DE (US); Jeffrey Keith Long, Wilmington, DE (US); Andrew Edmund Taggi, Newark, DE (US); Vijayagopal Gopalsamuthiram, Tamil Nadu (IN)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. Du Pont De Nemours and Company, Wilmington, DE (US)

(21) Appl. No.: 12/988,888

(22) PCT Filed: May 7, 2009
(86) PCT No.: PCT/US2009/043096

§ 371 Date: Oct. 21, 2010

(30) Foreign Application Priority Data
May 6, 2009 (US) PCT/US1994/000922

(51) Int. Cl. A01N 59/20; C07D 233/68; A01N 43/50; A01P 3/00; C07D 233/64; C07D 401/04; C07D 403/04; A01N 43/78; C07D 417/04; A01N 43/56; C07D 231/12; C07D 231/20; A01N 43/647; C07D 249/04; A01N 43/828; A01N 43/653; A01N 43/66; A01N 43/54; A01N 43/88

(52) U.S. Cl. 424/632; 548/343.1; 514/396; 514/400; 548/343.5; 546/272.7; 514/340; 548/313.4; 514/397; 514/365; 548/202; 546/167; 514/314; 514/406; 548/377.1; 548/366.1; 514/407; 514/359; 548/255; 544/333; 514/256; 514/361; 514/384; 514/245; 514/269; 514/367; 514/222.5; 424/637; 424/633

(57) ABSTRACT

Disclosed are compounds of Formula 1, including all geometric and stereoisomers, N-oxides, and salts thereof,

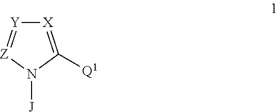

wherein
J is Q² or R¹;
X is N, CR² or CQ³;
Y is N or CR³;
Z is N or CR⁴; and
Q¹, Q², Q³, R¹ R² and R³ are as defined in the disclosure.
   Also disclosed are compositions containing the compounds of Formula 1 and methods for controlling plant disease caused by a fungal pathogen comprising applying an effective amount of a compound or a composition of the invention.

### FIELD OF THE INVENTION

This invention relates to certain azoles, their N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

Almansa et al., Journal of Medical Chemistry 2003, 46, 3463-3475 disclose certain 1,5-diarylimidazole derivatives and their use as cyclooxygenase-2 (COX-2) inhibitors.

Suketaka et al., Bulletin of the Chemical Society of Japan 1984, 57(2), 544-547 disclose certain 3,4-diaryl-4H-1,2,4-triazoles.

### SUMMARY OF THE INVENTION

This invention is directed to compounds of Formula 1 (including all geometric and stereoisomers), N-oxides, and salts thereof, agricultural compositions containing them and their use as fungicides:

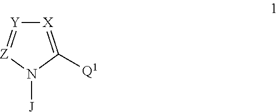

wherein
J is Q² or R¹;
X is N, CR² or CQ³;
Y is N or CR³;
Z is N or CR⁴;
Q¹ is a phenyl ring or a naphthalenyl ring system, each ring or ring system optionally substituted with up to 5 substituents independently selected from R^{5a}; or a 5- to 6-membered fully unsaturated heterocyclic ring or an 8- to 10-membered heteroaromatic bicyclic ring system, each ring or ring system containing ring members selected from carbon atoms and up to 4 heteroatoms independently selected from up to 2O, up to 2S and up to 4N atoms, wherein up to 3 carbon atom ring members are independently selected from C(═O) and C(═S), and the sulfur atom ring members are independently selected from S(═O)ₚ(═NR⁶)_{f}, each ring or ring system optionally substituted with up to 5 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W¹;
W¹ is a phenyl ring optionally substituted with up to 5 substituents independently selected from R^{5a}; or a 5- to 6-membered fully unsaturated heterocyclic ring containing ring members selected from carbon atoms and up to 4 heteroatoms independently selected from up to 2O, up to 2S and up to 4N atoms, wherein up to 2 carbon atom ring members are independently selected from C(═O) and C(═S), and the sulfur atom ring members are independently selected from S(═O)_{P} (═NR⁶)_{f}, the ring optionally substituted with up to 5 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members;
Q² is a phenyl ring or a naphthalenyl ring system, each ring or ring system optionally substituted with up to 5 substituents independently selected from R^{5b}; or a 5- to 6-membered fully unsaturated heterocyclic ring or an 8- to 10-membered heteroaromatic bicyclic ring system, each ring or ring system containing ring members selected from carbon atoms and up to 4 heteroatoms independently selected from up to 2O, up to 2S and up to 4N atoms, wherein up to 3 carbon atom ring members are independently selected from C(═O) and C(═S), and the sulfur atom ring members are independently selected from S(═O)ₚ(═NR⁶)_{f}, each ring or ring system optionally substituted with up to 5 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W²;
W² is a phenyl ring optionally substituted with up to 5 substituents independently selected from R^{5b}; or a 5- to 6-membered fully unsaturated heterocyclic ring containing ring members selected from carbon atoms and up to 4 heteroatoms independently selected from up to 2O, up to 2S and up to 4N atoms, wherein up to 2 carbon atom ring members are independently selected from C(═O) and C(═S), and the sulfur atom ring members are independently selected from S(═O)_{P} (═NR⁶)_{f}, the ring optionally substituted with up to 5 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members;
Q³ is a phenyl ring or a naphthalenyl ring system, each ring or ring system optionally substituted with up to 5 substituents independently selected from R^{5c}; or a 5- to 6-membered fully unsaturated heterocyclic ring or an 8- to 10-membered heteroaromatic bicyclic ring system, each ring or ring system containing ring members selected from carbon atoms and up to 4 heteroatoms independently selected from up to 2O, up to 2S and up to 4N atoms, wherein up to 3 carbon atom ring members are independently selected from C(═O) and C(═S), and the sulfur atom ring members are independently selected from S(═O)ₚ(═NR⁶)_{f}, each ring or ring system optionally substituted with up to 5 substituents independently selected from R^{5c} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W³;
W³ is a phenyl ring optionally substituted with up to 5 substituents independently selected from R^{5c}; or a 5- to 6-membered fully unsaturated heterocyclic ring containing ring members selected from carbon atoms and up to 4 heteroatoms independently selected from up to 2O, up to 2S and up to 4N atoms, wherein up to 2 carbon atom ring members are independently selected from C(═O) and C(═S), and the sulfur atom ring members are independently selected from S(═O)_{P} (═NR⁶)_{f}, the ring optionally substituted with up to 5 substituents independently selected from R^{5c} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members;
R¹ is C₁-C₇ alkyl, C₂-C₇ alkenyl, C₂-C₇ alkynyl, C₁-C₇ haloalkyl, C₂-C₇ haloalkenyl, C₃-C₇ cycloalkyl, C₃-C₇ halocycloalkyl, C₄-C₁₀ alkylcycloalkyl, C₄-C₁₀ cycloalkylalkyl, C₆-C₁₄ cycloalkylcycloalkyl, C₁-C₇ alkoxy, C₁-C₇ haloalkoxy, C₂-C₇ alkoxyalkyl, C₁-C₇ alkylthio, C₁-C₇ haloalkylthio, C₂-C₇ alkylthioalkyl, C₁-C₇ alkylsulfinyl, C₁-C₇ alkylsulfonyl, C₁-C₇ haloalkylsulfinyl, C₁-C₇ haloalkylsulfonyl, C₁-C₇ alkylamino, C₂-C₇ dialkylamino, C₂-C₇ alkylcarbonylamino or C₁-C₇ hydroxyalkyl;
each R², R³ and R⁴ is independently H, halogen, cyano, amino, nitro, —CHO, C₂-C₇ alkenyl, C₂-C₇ alkynyl, C₂-C₇ haloalkenyl, C₃-C₇ cycloalkyl, C₃-C₇ halocycloalkyl, C₄-C₁₀ alkylcycloalkyl, C₄-C₁₀ cycloalkylalkyl, C₆-C₁₄ cycloalkylcycloalkyl, C₁-C₇ alkoxy, C₁-C₇ haloalkoxy, C₂-C₇ alkoxyalkyl, C₁-C₇ alkylthio, C₁-C₇ haloalkylthio, C₂-C₇ alkylthioalkyl, C₁-C₇ alkylsulfinyl, C₁-C₇ alkylsulfonyl, C₁-C₇ haloalkylsulfinyl, C₁-C₇ haloalkylsulfonyl, C₁-C₇ alkylamino, C₂-C₇ dialkylamino, C₁-C₇ hydroxyalkyl, —SCN or CH═NOR¹¹; or C₁-C₇ alkyl or C₁-C₇ haloalkyl, each optionally substituted with up to 3 substituents independently selected from hydroxy, cyano, C(═O)OR⁸, C(═O)NR^{9a}R^{9b}, C(═O)R¹⁰ and CH═NOR¹¹;
each R^{5a}, R^{5b} and R^{5c} is independently halogen, cyano, hydroxy, nitro, C₁-C₇ alkyl, C₂-C₇ alkenyl, C₂-C₇ alkynyl, C₁-C₇ haloalkyl, C₂-C₇ haloalkenyl, C₃-C₇ cycloalkyl, C₃-C₇ halocycloalkyl, C₄-C₁₀ alkylcycloalkyl, C₄-C₁₀ cycloalkylalkyl, C₆-C₁₄ cycloalkylcycloalkyl, C₃-C₇ cycloalkoxy, C₃-C₇ halocycloalkoxy, C₁-C₇ alkoxy, C₁-C₇ haloalkoxy, C₁-C₆ alkylthio, C₁-C₇ haloalkylthio, C₁-C₇ alkylsulfinyl, C₁-C₇ alkylsulfonyl, C₁-C₇ haloalkylsulfinyl, C₁-C₇ haloalkylsulfonyl, C₁-C₇ alkylamino, C₂-C₇ dialkylamino, C₂-C₇ alkylcarbonyl, C₂-C₇ alkoxycarbonyl, C₂-C₇ alkylcarbonylamino, C₃-C₁₀ trialkylsilyl, SF₅, —SCN, C(═S)NH₂ or -U-V-T;
each U is independently O, S(═O)ₙ, NR¹² or a direct bond;
each V is independently C₁-C₆ alkylene, C₂-C₆ alkenylene, C₃-C₆ alkynylene, C₃-C₆ cycloalkylene or C₃-C₆ cycloalkenylene, wherein up to 3 carbon atoms are independently selected from C(═O), each optionally substituted with up to 5 substituents independently selected from halogen, cyano, nitro, hydroxy, C₁-C₆ alkyl, C₁-C₆ haloalkyl, C₁-C₆ alkoxy and C₁-C₆ haloalkoxy;
each T is independently NR^{13a}R^{13b}, OR¹⁴ or S(═O)ₙR¹⁴;
each R^{7a} is independently H, cyano or C₁-C₄ alkyl;
each R^{7b} is independently H or C₁-C₄ alkyl; or
a pair of R^{7a} and R^{7b} attached to the same carbon atom are taken together with the carbon atom to form a 3- to 6-membered saturated carbocyclic ring;
each R⁶ is independently H, cyano, C₁-C₃ alkyl or C₁-C₃ haloalkyl;
each R⁸, R^{9a}, R^{9b}, R¹⁰ and R¹¹ is independently H, C₁-C₇ alkyl, C₂-C₇ alkenyl, C₂-C₇ alkynyl, C₁-C₇ haloalkyl, C₂-C₇ haloalkenyl, C₃-C₇ cycloalkyl or C₃-C₇ halocycloalkyl;
each R¹² is independently H, C₁-C₆ alkyl, C₁-C₆ haloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R^{13a} and R^{13b} is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl); or
a pair of R^{13a} and R^{13b} attached to the same nitrogen atom are taken together with the nitrogen atom to form a 3- to 6-membered heterocyclic ring, the ring optionally substituted with up to 5 substituents independently selected from R¹⁵;
each R¹⁴ is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R¹⁵ is independently halogen, C₁-C₆ alkyl, C₁-C₆ haloalkyl or C₁-C₆ alkoxy;
each n is independently 0, 1 or 2; and
each p and f are independently 0, 1 or 2 in each instance of S(═O)ₚ(═NR⁶)_{f}, provided that the sum of p and f is 0, 1 or 2;
   provided that:
(a) when J is R¹, then X is CQ³;
(b) when J is Q², then X is N or CR², and if X is N or CH, then Z is other than CH;
(c) for compounds other than 1-(4-chlorophenyl)-5-(4-fluorophenyl)-2-methyl-1H-imidazole or 4-chloro-1-(4-chlorophenyl)-5-(4-fluorophenyl)-2-methyl-1H-imidazole, when Q¹ is a phenyl ring which is unsubstituted by R^{5a} at both ortho positions, then when X is N or CR² and Q² is a phenyl ring, the Q² phenyl ring is substituted by at least one R^{5b} at an ortho position; and when X is CQ³ and Q³ is a phenyl ring, the Q³ phenyl ring is substituted by at least one R^{5c} at an ortho position;
(d) at least one and no more than two of X, Y and Z is nitrogen;
(e) the compound is not a compound of F-1 through F-4, as shown below

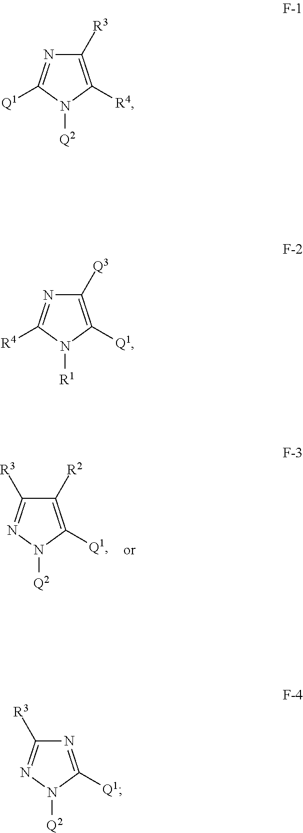

(g) the compound is not 4-[2-ethyl-1-(4-methoxyphenyl)-1H-imidazol-5-yl]-pyridine, 4-[1-(4-methoxyphenyl)-2-methyl-1H-imidazol-5-yl]pyridine or 3,5-dichloro-2-(4-iodo-5-phenyl-1H-1,2,3-triazol-1-yl)pyridine; and
(e) when J is Q², X is CR², Y is N and Z is N, then R² is other than H.

More particularly, this invention pertains to a compound of Formula 1 (including all geometric and stereoisomers), an N-oxide or a salt thereof.

This invention also relates to a fungicidal composition comprising a fungicidally effective amount of a compound of Formula 1 (or an N-oxide or salt thereof) and at least one additional component selected from the group consisting of surfactants, solid diluents and liquid diluents.

This invention also relates to a fungicidal composition comprising a mixture of a compound of Formula 1 (or an N-oxide or salt thereof) and at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of the invention (e.g., as a composition described herein).

### DETAILS OF THE INVENTION

As used herein, the terms “comprises,” “comprising,” “includes,” “including,” “has,” “having”, “contains” or “containing” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

As referred to herein, the term “broadleaf” used either alone or in words such as “broadleaf crop” means dicot or dicotyledon, a term used to describe a group of angiosperms characterized by embryos having two cotyledons.

As used herein, the term “alkylating agent” refers to a chemical compound in which a carbon-containing radical is bound through a carbon atom to leaving group such as halide or sulfonate, which is displaceable by bonding of a nucleophile to said carbon atom. Unless otherwise indicated, the term “alkylating” does not limit the carbon-containing radical to alkyl; the carbon-containing radicals in alkylating agents include the variety of carbon-bound substituent radicals specified, for example, for R², R³ and R⁴.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl, hexyl or heptyl isomers. “Alkenyl” includes straight-chain or branched alkenes such as ethenyl, 1-propenyl, 2-propenyl, and the different butenyl, pentenyl, hexenyl and heptenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl, hexynyl and heptynyl isomers. “Alkynyl” can also include moieties comprised of multiple triple bonds such as 2,5-hexadiynyl. “Alkylene” denotes a straight-chain or branched alkanediyl. Examples of “alkylene” include CH₂, CH₂CH₂, CH(CH₃), CH₂CH₂CH₂, CH₂CH(CH₃) and the different butylene, pentylene and hexylene isomers. “Alkenylene” denotes a straight-chain or branched alkenediyl containing one olefinic bond. Examples of “alkenylene” include CH═CH, CH₂CH═CH, CH═C(CH₃). “Alkynylene” denotes a straight-chain or branched alkynediyl containing one triple bond. Examples of “alkynylene” include CH₂C≡C, C≡CCH₂ and the different butynylene, pentynylene and hexynylene isomers.

“Alkoxy” includes, for example, methoxy, ethoxy, n-propyloxy, isopropyloxy and the different butoxy, pentoxy, hexyloxy and heptyloxy isomers. “Alkylthio” includes branched or straight-chain alkylthio moieties such as methylthio, ethylthio, and the different propylthio, butylthio, pentylthio, hexylthio and heptylthio isomers. “Alkylsulfinyl” includes both enantiomers of an alkylsulfinyl group. Examples of “alkylsulfinyl” include CH₃S(═O), CH₃CH₂S(═O), CH₃CH₂CH₂S(═O), (CH₃)₂CHS(═O) and the different butylsulfinyl, pentylsulfinyl, hexylsulfinyl and heptylsulfinyl isomers. Examples of “alkylsulfonyl” include CH₃S(═O)₂, CH₃CH₂S(═O)₂, CH₃CH₂CH₂S(═O)₂, (CH₃)₂CHS(═O)₂, and the different butylsulfonyl, pentylsulfonyl, hexylsulfonyl and heptylsulfonyl isomers. “Alkylamino” includes an NH radical substituted with straight-chain or branched alkyl. Examples of “alkylamino” include CH₃CH₂NH, CH₃CH₂CH₂NH, and (CH₃)₂CHCH₂NH. Examples of “dialkylamino” include (CH₃)₂N, (CH₃CH₂CH₂)₂N and CH₃CH₂(CH₃)N.

“Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂; “alkylsulfinylalkyl” and “alkylsulfonylalkyl” include the corresponding sulfoxides and sulfones, respectively. “(Alkylthio)carbonyl” denotes a straight-chain or branched alkylthio group bonded to a C(═O) moiety. Examples of “(alkylthio)carbonyl” include CH₃SC(═O), CH₃CH₂CH₂SC(═O) and (CH₃)₂CHSC(═O). “Alkoxy(thiocarbonyl)” denotes a straight-chain or branched alkoxy group bonded to a C(═S) moiety. Examples of “alkoxy(thiocarbonyl)” include CH₃C(═S), CH₃CH₂CH₂C(═S) and (CH₃)₂CHOC(═S). “Alkylaminoalkyl” denotes alkylamino substitution on alkyl. Examples of “alkylaminoalkyl” include CH₃NHCH₂, CH₃NHCH₂CH₂, CH₃CH₂NHCH₂, CH₃CH₂CH₂CH₂NHCH₂ and CH₃CH₂NHCH₂CH₂. Examples of “dialkylaminoalkyl” include ((CH₃)₂CH)₂NCH₂, (CH₃CH₂CH₂)₂NCH₂ and CH₃CH₂(CH₃)NCH₂CH₂. The term “alkylcarbonylamino” denotes alkyl bonded to a C(═O)NH moiety. Examples of “alkylcarbonylamino” include CH₃CH₂C(═O)NH and CH₃CH₂CH₂C(═O)NH.

“Hydroxyalkyl” denotes an alkyl group substituted with one hydroxy group. Examples of “hydroxyalkyl” include HOCH₂CH₂, CH₃CH₂(OH)CH and HOCH₂CH₂CH₂CH₂.

“Cycloalkyl” includes, for example, cyclopropyl, cyclobutyl, cyclopentyl, cyclohexyl and cycloheptyl. The term “alkylcycloalkyl” denotes alkyl substitution on a cycloalkyl moiety and includes, for example, ethylcyclopropyl, i-propylcyclobutyl, methylcyclopentyl and methylcyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. The term “cycloalkylcycloalkyl” denotes cycloalkyl substitution on another cycloalkyl ring, wherein each cycloalkyl ring independently has from 3 to 7 carbon atom ring members. Examples of cycloalkylcycloalkyl include cyclopropylcyclopropyl (such as 1,1′-bicyclopropyl-1-yl, 1,1′-bicyclopropyl-2-yl), cyclohexylcyclopentyl (such as 4-cyclopentylcyclohexyl) and cyclohexylcyclohexyl (such as 1,1′-bicyclohexyl-1-yl), and the different cis- and trans-cycloalkylcycloalkyl isomers, (such as (1R,2S)-1,1′-bicyclopropyl-2-yl and (1R,2R)-1,1′-bicyclopropyl-2-yl). The term “cycloalkoxy” denotes cycloalkyl attached to and linked through an oxygen atom including, for example, cyclopentyloxy and cyclohexyloxy. “Cycloalkylcarbonyl” denotes cycloalkyl bonded to a C(═O) group including, for example, cyclopropylcarbonyl and cyclopentylcarbonyl. The term “cycloalkoxycarbonyl” means cycloalkoxy bonded to a C(═O) group, for example, cyclopropyloxycarbonyl and cyclopentyloxycarbonyl. The term“cycloalkylene” denotes a cycloalkanediyl ring. Examples of “cycloalkylene” include cyclopropylene, cyclobutylene, cyclopentylene and cyclohexylene. The term “cycloalkenylene” denotes a cycloalkenediyl ring containing one olefinic bond. Examples of “cycloalkenylene” include cylopropenediyl and cyclpentenediyl.

Examples of “alkylene” include CH₂, CH₂CH₂, CH(CH₃), CH₂CH₂CH₂, CH₂CH(CH₃) and the different butylene, pentylene and hexylene isomers.

“Alkylcarbonyl” denotes a straight-chain or branched alkyl bonded to a C(═O) moiety. Examples of “alkylcarbonyl” include CH₃C(═O), CH₃CH₂CH₂C(═O) and (CH₃)₂CHC(═O). Examples of “alkoxycarbonyl” include CH₃C(═O), CH₃CH₂C(═O), CH₃CH₂CH₂C(═O), (CH₃)₂CHOC(═O) and the different butoxy-, pentoxy-, hexoxy- and heptoxycarbonyl isomers.

“Trialkylsilyl” includes 3 branched and/or straight-chain alkyl radicals attached to and linked through a silicon atom, such as trimethylsilyl, triethylsilyl and tert-butyldimethylsilyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “haloalkenyl”, “haloalkoxy”, “haloalkylthio”, “haloalkylsulfinyl”, “haloalkylsulfonyl”, “halocycloalkyl”, and the like, are defined analogously to the term “haloalkyl”. Examples of “haloalkenyl” include Cl₂C═CHCH₂ and CF₃CH₂CH═CHCH₂. Examples of “haloalkoxy” include CF₃O, CCl₃CH₂O, F₂CHCH₂CH₂O and CF₃CH₂O. Examples of “haloalkylthio” include CCl₃S, CF₃S, CCl₃CH₂S and ClCH₂CH₂CH₂S. Examples of “haloalkylsulfinyl” include CF₃S(═O), CCl₃S(═O), CF₃CH₂S(═O) and CF₃CF₂S(═O). Examples of “haloalkylsulfonyl” include CF₃S(═O)₂, CCl₃S(═O)₂, CF₃CH₂S(═O)₂ and CF₃CF₂S(═O)₂. Examples of “halocycloalkyl” include 2-chlorocyclopropyl, 2-fluorocyclobutyl, 3-bromocyclopentyl and 4-chorocyclohexyl.

The total number of carbon atoms in a substituent group is indicated by the “Cᵢ-Cⱼ” prefix where i and j are numbers from 1 to 14. For example, C₁-C₄ alkylsulfonyl designates methylsulfonyl through butylsulfonyl; C₂ alkoxyalkyl designates CH₃OCH₂; C₃ alkoxyalkyl designates, for example, CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

The term “unsubstituted” in connection with a group such as a ring or ring system means the group does not have any substituents other than its one or more attachments to the remainder of Formula 1. The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) ranges from 1 to 3. As used herein, the term “optionally substituted” is used interchangeably with the phrase “substituted or unsubstituted” or with the term “(un)substituted.”

The number of optional substituents may be restricted by an expressed limitation. For example, the phrase “optionally substituted with up to 3 substituents selected from R^{5a} on carbon ring members” means that 0, 1, 2 or 3 substituents can be present (if the number of potential connection points allows). Similarly, the phrase “optionally substituted with up to 5 substituents selected from R^{5a} on carbon ring members” means that 0, 1, 2, 3, 4 or 5 substituents can be present if the number of available connection points allows. When a range specified for the number of substituents (e.g., r being an integer from 0 to 5 in Exhibit 1) exceeds the number of positions available for substituents on a ring (e.g., 2 positions available for (R^{v})ᵣ on A-11 in Exhibit 1), the actual higher end of the range is recognized to be the number of available positions.

When a compound is substituted with a substituent bearing a subscript that indicates the number of said substituents can exceed 1, said substituents (when they exceed 1) are independently selected from the group of defined substituents (e.g., (R^{v}), wherein r is 1, 2, 3, 4 or 5 in Exhibit 1). When a variable group is shown to be optionally attached to a position, for example (R^{v})ᵣ wherein r may be 0, then hydrogen may be at the position even if not recited in the variable group definition. When one or more positions on a group are said to be “not substituted” or “unsubstituted”, then hydrogen atoms are attached to take up any free valency.

Unless otherwise indicated, a “ring” as a component of Formula 1 is carbocyclic or heterocyclic. The term “ring system” as a component of Formula 1 denotes two fused rings (e.g., two phenyl rings fused to form naphthalenyl). The term “ring member” refers to an atom (e.g., C, O, N or S) or other moiety (e.g., C(═O), C(═S) or S(═O)ₚ(═NR⁶)_{f}) forming the backbone of a ring or ring system.

The term “nonaromatic” includes rings that are fully saturated as well as partially or fully unsaturated, provided that none of the rings are aromatic. In particular, a “fully unsaturated heterocycle” includes both aromatic and nonaromatic heterocycles. The term “aromatic” indicates that each of the ring atoms of a fully unsaturated ring is essentially in the same plane and has a p-orbital perpendicular to the ring plane, and that (4n+2) π electrons, where n is a positive integer, are associated with the ring to comply with Hückel's rule.

The terms “carbocyclic ring”, “carbocycle” or “carbocyclic ring system” denote a ring or ring system wherein the atoms forming the ring backbone are selected only from carbon. Unless otherwise indicated, a carbocyclic ring can be a saturated, partially unsaturated, or fully unsaturated ring. When a fully unsaturated carbocyclic ring satisfies Hückel's rule, then said ring is also called an “aromatic carbocyclic ring”. “Saturated carbocyclic” refers to a ring having a backbone consisting of carbon atoms linked to one another by single bonds; unless otherwise specified, the remaining carbon valences are occupied by hydrogen atoms.

The terms “heterocyclic ring” or “heterocycle” denote a ring in which at least one atom forming the ring backbone is not carbon (e.g., N, O or S). Typically a heterocyclic ring contains no more than 4N atoms, no more than 2O atoms and no more than 2S atoms. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring” or “aromatic heterocyclic ring”. The terms “heteroaromatic ring system” or “heteroaromatic bicyclic ring system” denote a ring system in which at least one atom forming the ring backbone is not carbon (e.g., N, O or S) and at least one ring is aromatic. Unless otherwise indicated, heterocyclic rings and heteroaromatic ring systems can be attached through any available carbon or nitrogen by replacement of a hydrogen on said carbon or nitrogen.

In the context of the present invention when an instance of Q¹, Q² and Q³ comprises a phenyl or a 5- to 6-membered fully unsaturated heterocyclic ring, the ortho, meta and para positions of each ring is relative to the connection of the ring to the remainder of Formula 1. Further, when an instance of Q¹, Q² and Q³ comprises a phenyl or a 5- to 6-membered fully unsaturated heterocyclic ring attached through the linker CR^{7a}R^{7b} to the remained of Formula 1, the ortho, meta and para positions of each ring is relative to the connection of the ring to the linker CR^{7a}R^{7b}.

As noted above, each Q¹, Q² and Q³ is, inter alia, a 5- to 6-membered fully unsaturated heterocyclic ring or an 8- to 10-membered heteroaromatic bicyclic ring system, each ring or ring system containing ring members selected from carbon atoms and up to 4 heteroatoms independently selected from up to 2O, up to 2S and up to 4N atoms, wherein up to 3 carbon atom ring members are independently selected from C(═O) and C(═S), the sulfur atom ring members are independently selected from S(═O)ₚ(═NR⁶)_{f}, each ring or ring system optionally substituted with up to 5 substituents independently selected from any substituent defined in the Summary of the Invention for Q¹, Q² and Q³ (e.g., a Q¹ ring or ring system is optionally substituted with R^{5a} on carbon ring members and cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members). As the substituents are optional, 0 to 5 substituents may be present, limited only by the number of available points of attachment. In this definition the ring members selected from up to 2O, up to 2S and up to 4N atoms are optional, provided at least one ring member is not carbon (e.g., N, O or S). The definition of S(═O)_{P}(═NR⁶)_{f} allows the up to 2 sulfur ring members, to be oxidized sulfur moieties (e.g., S(═O) or S(═O)₂) or unoxidized sulfur atoms (i.e. when p and f are both zero). The nitrogen atom ring members may be oxidized as N-oxides, because compounds relating to Formula 1 also include N-oxide derivatives. The up to 3 carbon atom ring members selected from C(═O) and C(═S) are in addition to the up to 4 heteroatoms selected from up to 2O, up to 2S and up to 4N atoms. Examples of a 5- to 6-membered fully unsaturated heterocyclic ring include the rings A-1 through A-31 illustrated in Exhibit 1, and examples of an 8- to 10-membered heteroaromatic bicyclic ring system include the ring systems A-31 through A-72 illustrated in Exhibit 2. In Exhibits 1 and 2 the relative the variable (R^{v})ᵣ is any substituent as defined in the Summary of the Invention for Q¹, Q² and Q³ (e.g., a Q¹ ring or ring system is optionally substituted with R^{5a} on carbon ring members and cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members) and r is an integer from 0 to 5, limited by the number of available positions on each depicted ring or ring system.

As noted above, each W¹, W² and W³ is, inter alia, a 5- to 6-membered fully unsaturated heterocyclic ring containing ring members selected from carbon atoms and up to 4 heteroatoms independently selected from up to 2O, up to 2S and up to 4N atoms, wherein up to 2 carbon atom ring members are independently selected from C(═O) and C(═S), the sulfur atom ring members are independently selected from S(═O)ₚ(═NR⁶)_{f}, the ring optionally substituted with up to 5 substituents independently selected from any substituent defined in the Summary of the Invention for W¹, W² and W³ (e.g., a W¹ ring is optionally substituted with R^{5a} on carbon ring members and cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members). As the substituents are optional, 0 to 5 substituents may be present, limited only by the number of available points of attachment. In this definition the ring members selected from up to 2O, up to 2S and up to 4N atoms are optional, provided at least one ring member is not carbon (e.g., N, O or S). The definition of S(═O)ₚ(═NR⁶)_{f} allows the up to 2 sulfur ring members, to be oxidized sulfur moieties (e.g., S(═O) or S(═O)₂) or unoxidized sulfur atoms (i.e. when p and f are both zero). The nitrogen atom ring members may be oxidized as N-oxides, because compounds relating to Formula 1 also include N-oxide derivatives. The up to 2 carbon atom ring members selected from C(═O) and C(═S) are in addition to the up to 4 heteroatoms selected from up to 2O, up to 2S and up to 4N atoms. Examples of a 5- to 6-membered fully unsaturated heterocyclic ring in W¹, W² and W³ include the rings A-1 through A-31 illustrated in Exhibit 1 wherein (R^{v})ᵣ is any substituent as defined in the Summary of the Invention for W¹, W² or W³ (e.g., a W¹ ring is optionally substituted with R^{5a} on carbon ring members and cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members) and r is an integer from 0 to 5, limited by the number of available positions on each A-ring.

Although R^{v} groups are shown in the structures A-1 through A-72, it is noted that they do not need to be present since they are optional substituents. Note that when R^{v} is H attached to an atom, this is the same as if said atom is unsubstituted. The nitrogen atoms that require substitution to fill their valence are substituted with H or R^{v}. Note that when the attachment point between (R^{v})ᵣ and the depicted ring or ring system is illustrated as floating, (R^{v})ᵣ can be attached to any available carbon atom or nitrogen atom of the depicted ring or ring system. Note that when the attachment point on the depicted ring or ring system is illustrated as floating, the depicted ring or ring system can be attached to the remainder of Formula 1 through any available carbon or nitrogen of the depicted ring or ring system by replacement of a hydrogen atom.

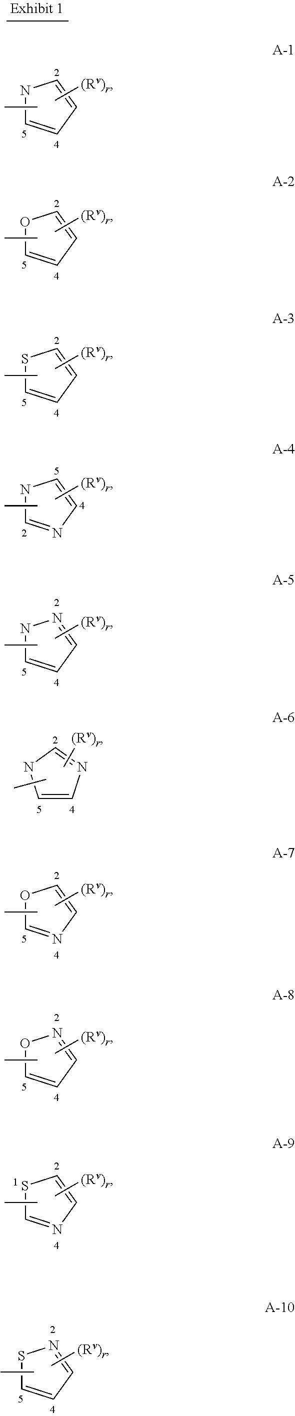

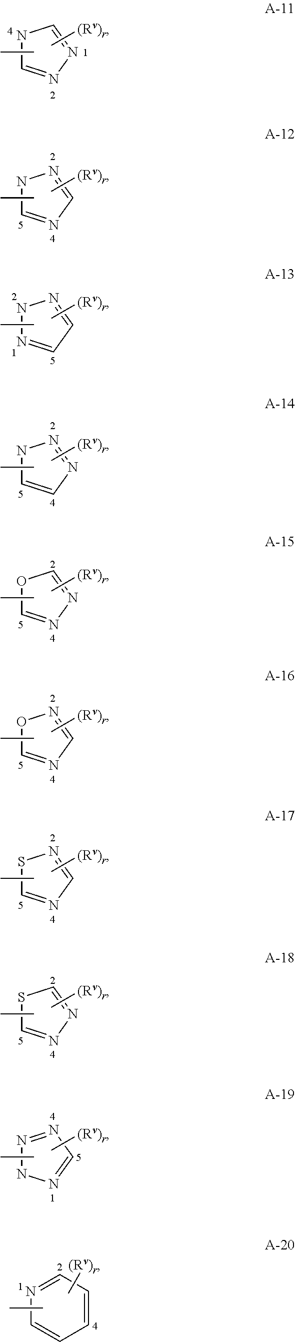

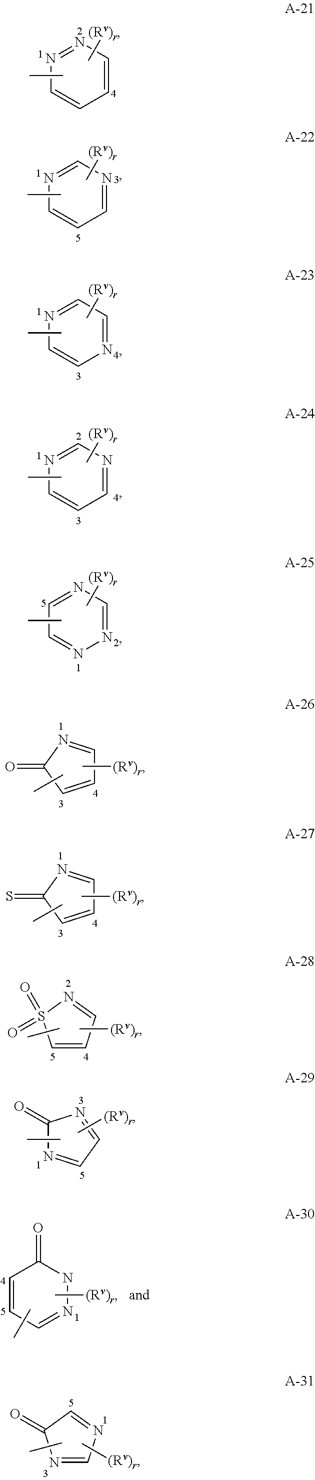

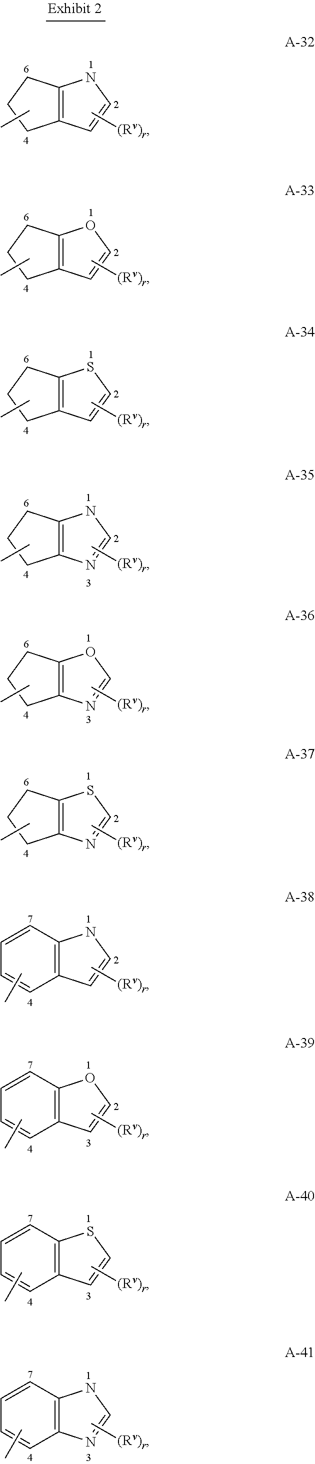

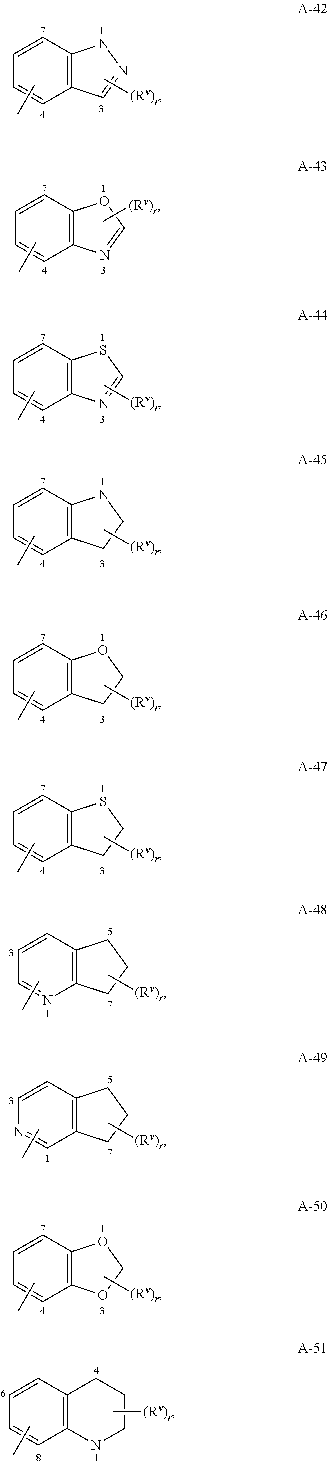

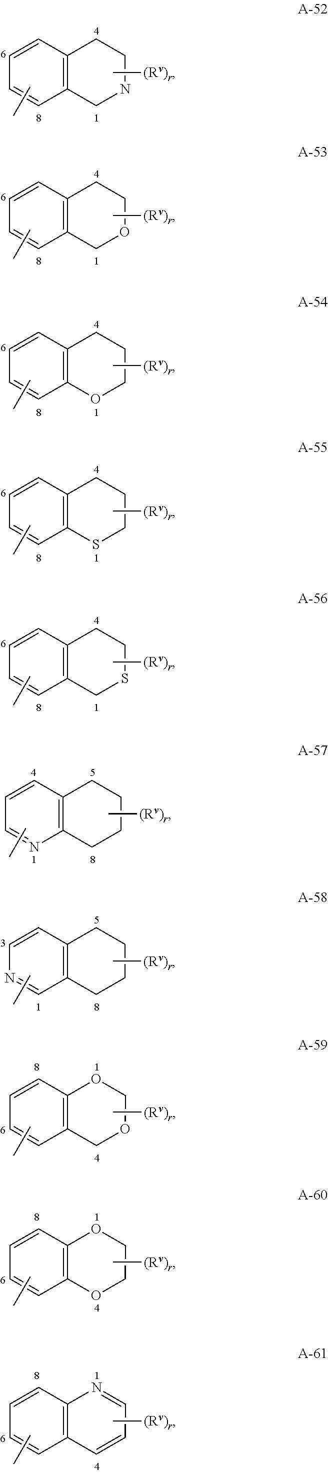

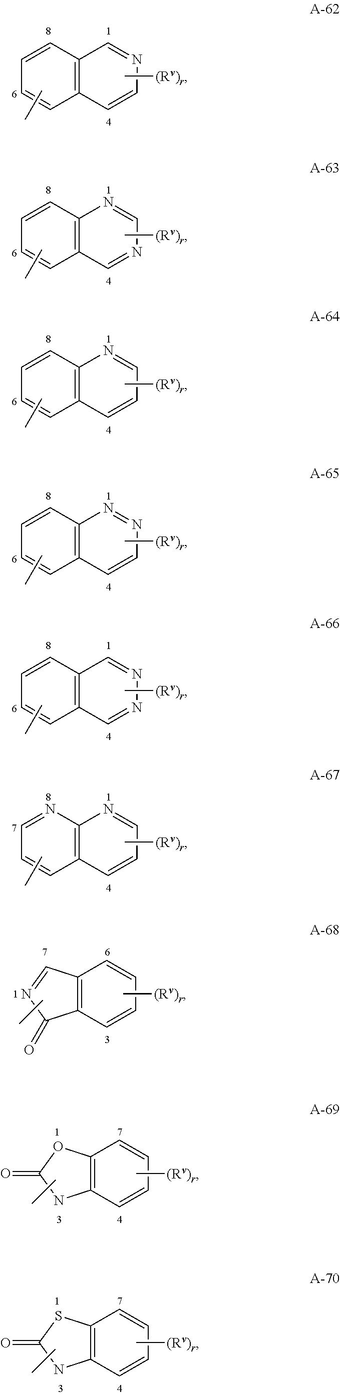

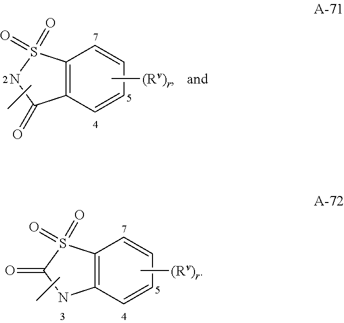

A wide variety of synthetic methods are known in the art to enable preparation of aromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers or as individual stereoisomers (e.g., in optically active form). Of note are atropisomers, which are conformational isomers that occur when rotation about a single bond in a molecule is restricted as a result of steric interaction with other parts of the molecule and the substituents at both ends of the single bond are unsymmetrical. In the present invention, atropisomerism occurs at a single bond in Formula 1 when the rotational barrier is high enough (about ΔG>25 kcal mol⁻¹) that separation of isomers at ambient temperature becomes possible. One skilled in the art will appreciate that one atropisomer may be more active and/or may exhibit beneficial effects when enriched relative to the other atropisomer or when separated from the other atropisomer. Additionally, the skilled artisan knows how to separate, enrich, and/or to selectively prepare said atropisomers. A detailed description of atropisomers can be found in March, Advanced Organic Chemistry, 4th Ed. 1992, 101-102 and Gawronski et al, Chirality 2002, 14, 689-702. This invention includes compounds or compositions that are enriched in an atropisomer of Formula 1 compared to other atropisomers of the compounds. Also included are the essentially pure atropisomers of compounds of Formula 1.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. Accordingly, the present invention comprises compounds selected from Formula 1, N-oxides and agriculturally suitable salts thereof.

Compounds selected from Formula 1, geometric and other stereoisomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

Embodiments of the present invention as described in the Summary of the Invention include those described below. In the following Embodiments, Formula 1 includes N-oxides and salts thereof, and reference to “a compound of Formula 1” includes the definitions of substituents specified in the Summary of the Invention unless further defined in the Embodiments.
Embodiment 1. A compound of Formula 1 wherein J is Q².
Embodiment 2. A compound of Formula 1 wherein J is R¹.
Embodiment 3. A compound of Formula 1 or Embodiment 2 wherein R¹ is C₁-C₃ alkyl.
Embodiment 4. A compound of Embodiment 3 wherein R¹ is methyl.
Embodiment 5. A compound of Formula 1 or any one of Embodiments 1 through 4 wherein X is CR² or CQ³.
Embodiment 6. A compound of Embodiment 5 wherein X is CR².
Embodiment 6a. A compound of Embodiment 5 wherein X is CQ³.
Embodiment 7. A compound of Formula 1 or any one of Embodiments 1 through 6a wherein Y is N.
Embodiment 8. A compound of Formula 1 or any one of Embodiments 1 through 6a wherein Y is CR³.
Embodiment 9. A compound of Formula 1 or any one of Embodiments 1 through 8 wherein Z is CR⁴.
Embodiment 10. A compound of Formula 1 or any one of Embodiments 1 through 8 wherein Z is N.
Embodiment 11. A compound of Formula 1 wherein J is Q², X is CR², Y is N and Z is CR⁴, or J is R¹, X is CQ³, Y is CR³ and Z is N, or J is Q², X is CR², Y is N and Z is N.
Embodiment 12. A compound of Embodiment 11 wherein J is Q², X is CR², Y is N and Z is CR⁴ or J is R¹, X is CQ³, Y is CR³ and Z is N.
Embodiment 13. A compound of Embodiment 12 wherein J is Q², X is CR², Y is N and Z is CR⁴.
Embodiment 14. A compound of Formula 1 or any one of Embodiments 1 through 13 wherein Q¹ is a phenyl ring or a naphthalenyl ring system, each ring or ring system optionally substituted with up to 3 substituents independently selected from R^{5a}; or a 5- to 6-membered fully unsaturated heterocyclic ring containing ring members selected from carbon atoms and up to 3 heteroatoms independently selected from up to 2O, up to 2S and up to 3N atoms, wherein up to 2 carbon atom ring members are independently selected from C(═O) and C(═S), and the sulfur atom ring members are independently selected from S(═O)ₛ(═NR⁶)_{f}, the ring optionally substituted with up to 3 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W¹.
Embodiment 15. A compound of Embodiment 14 wherein Q¹ is a phenyl, thienyl, pyridinyl, pyridazinyl, pyrimidinyl or pyrazolyl ring or a naphthalenyl ring system, each ring or ring system optionally substituted with up to 3 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W¹.
Embodiment 16. A compound of Embodiment 15 wherein Q¹ is a phenyl or pyridinyl ring, optionally substituted with up to 3 substituents independently selected from R^{5a}.
Embodiment 17. A compound of Embodiment 16 wherein Q¹ is a pyridinyl ring attached to Formula 1 at the 3-position of the pyridinyl ring and optionally substituted with up to 3 substituents independently selected from R^{5a}.
Embodiment 18. A compound of Embodiment 16 wherein Q¹ is a phenyl ring optionally substituted with up to 3 substituents independently selected from R^{5a}.
Embodiment 19. A compound of Formula 1 or any one of Embodiments 1 through 15 wherein W¹ is a phenyl, thienyl, pyridinyl, pyridazinyl, pyrimidinyl or pyrazolyl ring optionally substituted with up to 3 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members.
Embodiment 20. A compound of Embodiment 19 wherein W¹ is a phenyl or pyridinyl ring, optionally substituted with up to 3 substituents independently selected from R^{5a}.
Embodiment 21. A compound of Embodiment 20 wherein W¹ is a phenyl ring optionally substituted with up to 3 substituents independently selected from R^{5a}.
Embodiment 22. A compound of Formula 1 or any one of Embodiments 1 through 21 wherein Q² is a phenyl ring or a naphthalenyl ring system, each ring or ring system optionally substituted with up to 3 substituents independently selected from R^{5b}; or a 5- to 6-membered fully unsaturated heterocyclic ring containing ring members selected from carbon atoms and up to 3 heteroatoms independently selected from up to 2O, up to 2S and up to 3N atoms, wherein up to 2 carbon atom ring members are independently selected from C(═O) and C(═S), and the sulfur atom ring members are independently selected from S(═O)ₛ(═NR⁶)_{f}, the ring optionally substituted with up to 3 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W².
Embodiment 23. A compound of Embodiment 22 wherein Q² is a phenyl, thienyl, pyridinyl, pyridazinyl, pyrimidinyl or pyrazolyl ring or a naphthalenyl ring system, each ring or ring system optionally substituted with up to 3 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W².
Embodiment 24. A compound of Embodiment 23 wherein Q² is a phenyl or pyridinyl ring, optionally substituted with up to 3 substituents independently selected from R^{5b}.
Embodiment 25. A compound of Embodiment 24 wherein Q² is a pyridinyl ring attached to Formula 1 at the 3-position of the pyridinyl ring and optionally substituted with up to 3 substituents independently selected from R^{5b}.
Embodiment 26. A compound of Embodiment 24 wherein Q² is a phenyl ring optionally substituted with up to 3 substituents independently selected from R^{5b}.
Embodiment 27. A compound of Formula 1 or any one of Embodiments 1 through 26 wherein when each Q¹ and Q² is independently a phenyl or pyridinyl ring, then one of the Q¹ and Q² rings is substituted with 2 or 3 substituents and the other of the Q¹ and Q² rings is substituted with 1 or 3 substituents.
Embodiment 27a. A compound of Formula 1 or any one of Embodiments 1 through 27 wherein when each Q¹ and Q² is independently a phenyl or pyridinyl ring, then one of the Q¹ and Q² rings is substituted with 2 or 3 substituents and the other of the Q¹ and Q² rings is substituted with 1 or 2 substituents.
Embodiment 28. A compound of Formula 1 or any one of Embodiments 1 through 27 wherein at least one R^{5a} substituent is attached at an ortho position of the Q¹ ring.
Embodiment 29. A compound of Formula 1 or any one of Embodiments 1 through 28 wherein two R^{5a} substituents are attached at ortho positions of the Q¹ ring.
Embodiment 30. A compound of Formula 1 or any one of Embodiments 1 through 29 wherein at least one R^{5b} substituent is attached at an ortho position of the Q² ring.
Embodiment 31. A compound of Formula 1 or any one of Embodiments 1 through 30 wherein two R^{5b} substituents are attached at ortho positions of the Q² ring.
Embodiment 32. A compound of Formula 1 or any one of Embodiments 1 through 23 wherein W² is a phenyl, thienyl, pyridinyl, pyridazinyl, pyrimidinyl or pyrazolyl ring optionally substituted with up to 3 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members.
Embodiment 33. A compound of Embodiment 32 wherein W² is a phenyl or pyridinyl ring, optionally substituted with up to 3 substituents independently selected from R^{5b}.
Embodiment 34. A compound of Embodiment 33 wherein W² is a phenyl ring optionally substituted with up to 3 substituents independently selected from R^{5b}.
Embodiment 35. A compound of Formula 1 or any one of Embodiments 1 through 34 wherein Q³ is a phenyl ring or a naphthalenyl ring system, each ring or ring system optionally substituted with up to 3 substituents independently selected from R^{5c}; or a 5- to 6-membered fully unsaturated heterocyclic ring containing ring members selected from carbon atoms and up to 3 heteroatoms independently selected from up to 2O, up to 2S and up to 3N atoms, wherein up to 2 carbon atom ring members are independently selected from C(═O) and C(═S), and the sulfur atom ring members are independently selected from S(═O)ₛ(═NR⁶)_{f}, the ring optionally substituted with up to 3 substituents independently selected from R^{5c} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W³.
Embodiment 36. A compound of Embodiment 35 wherein Q³ is a phenyl, thienyl, pyridinyl, pyridazinyl, pyrimidinyl or pyrazolyl ring or a naphthalenyl ring system, each ring or ring system optionally substituted with up to 3 substituents independently selected from R^{5c} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W³.
Embodiment 37. A compound of Embodiment 36 wherein Q³ is a phenyl or pyridinyl ring, optionally substituted with up to 3 substituents independently selected from R^{5c}.
Embodiment 38. A compound of Embodiment 37 wherein Q³ is a pyridinyl ring attached to Formula 1 at the 3-position of the pyridinyl ring and optionally substituted with up to 3 substituents independently selected from R^{5c}.
Embodiment 39. A compound of Embodiment 37 wherein Q³ is a phenyl ring optionally substituted with up to 3 substituents independently selected from R^{5c}.
Embodiment 40. A compound of Formula 1 or any one of Embodiments 1 through 36 wherein W³ is a phenyl, thienyl, pyridinyl, pyridazinyl, pyrimidinyl or pyrazolyl ring optionally substituted with up to 3 substituents independently selected from R^{5c} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members.
Embodiment 41. A compound of Embodiment 40 wherein W³ is a phenyl or pyridinyl ring, optionally substituted with up to 3 substituents independently selected from R^{5c}.
Embodiment 42. A compound of Embodiment 41 wherein W³ is a phenyl ring optionally substituted with up to 3 substituents independently selected from R^{5c}.
Embodiment 43. A compound of Formula 1 or any one of Embodiments 1 through 42 wherein each R², R³ and R⁴ is independently H, halogen, C₂-C₃ alkenyl, C₂-C₃ alkynyl, C₂-C₃ haloalkenyl, C₃-C₆ cycloalkyl, C₁-C₃ alkoxy, C₁-C₃ alkylthio, C₁-C₃ alkylamino, C₂-C₄ dialkylamino or C₁-C₃ hydroxyalkyl; or C₁-C₃ alkyl or C₁-C₃ haloalkyl, each optionally substituted with up to 1 substituent independently selected from hydroxy, cyano, C(═O)OR⁸, C(═O)NR^{9a}R^{9b}, C(═O)R¹⁰ and CH═NOR¹¹.
Embodiment 44. A compound of Embodiment 43 wherein each R², R³ and R⁴ is independently H, halogen, cyano or C₁-C₃ alkyl.
Embodiment 45. A compound of Embodiment 44 wherein each R², R³ and R⁴ is independently Cl, Br, I or C₁-C₂ alkyl.
Embodiment 46. A compound of Embodiment 45 wherein each R², R³ and R⁴ is independently Cl, Br or methyl.
Embodiment 47. A compound of Formula 1 or any one of Embodiments 1 through 46 wherein each R^{5a}, R^{5b} and R^{5c} is independently halogen, cyano, C₁-C₃ alkyl, C₂-C₃ alkenyl, C₂-C₃ alkynyl, C₁-C₃ haloalkyl, C₃ cycloalkyl, C₁-C₃ alkoxy, C₁-C₃ haloalkoxy, C₁-C₃ alkylthio, C₁-C₃ alkylamino, C₂-C₄ dialkylamino C₂-C₄ alkylcarbonyl, C₂-C₄ alkoxycarbonyl, C₂-C₄ alkylcarbonylamino or -U-V-T.
Embodiment 48. A compound of Embodiment 47 wherein each R^{5a}, R^{5b} and R^{5c} is independently halogen, cyano, C₁-C₃ alkyl, C₂-C₃ alkenyl, C₁-C₃ haloalkyl, C₁-C₃ alkoxy, C₁-C₃ alkylthio or C₁-C₃ alkylamino.
Embodiment 49. A compound of Embodiment 48 wherein each R^{5a}, R^{5b} and R^{5c} is independently halogen, cyano, C₁-C₃ alkyl, C₁-C₃ haloalkyl or C₁-C₃ alkoxy.
Embodiment 50. A compound of Embodiment 49 wherein each R^{5a}, R^{5b} and R^{5c} is independently F, Cl, Br, cyano, C₁-C₂ alkyl, C₁-C₂ haloalkyl or C₁-C₂ alkoxy.
Embodiment 51. A compound of Embodiment 50 wherein each R^{5a}, R^{5b} and R^{5c} is independently F, Cl, cyano, methyl, trifluoromethyl or methoxy.
Embodiment 52. A compound of Formula 1 or any one of Embodiments 1 through 47 wherein each U is independently O or NR¹².
Embodiment 53. A compound of Embodiment 52 wherein each U is independently O or NH.
Embodiment 54. A compound of Formula 1 or any one of Embodiments 1 through 47 wherein each V is C₂-C₄ alkylene.
Embodiment 55. A compound of Formula 1 or any one of Embodiments 1 through 47 wherein each T is independently NR^{13a}R^{13b} or OR¹⁴.
Embodiment 55a. A compound of Formula 1 or any one of Embodiments 1 through 55 wherein each R^{13a} and R^{13b} is independently H, C₁-C₆ alkyl or C₁-C₆ haloalkyl.
Embodiment 55b. A compound of Formula 1 or any one of Embodiments 1 through 55 wherein each R¹⁴ is independently H, C₁-C₆ alkyl or C₁-C₆ haloalkyl.
Embodiment 56. A compound of Formula 1 or any one of Embodiments 1 through 55b independently when an instance of R^{7a} is not taken together with the carbon atom to which the instance of R^{7a} is attached and a geminal instance of R^{7b} to form a carbocyclic ring (i.e. R^{7a} is taken alone) then the instance of R^{7a} is H, cyano or methyl.
Embodiment 56a. A compound of Embodiment 56 wherein each R^{7a} is independently H or methyl.
Embodiment 57. A compound of Embodiment 56a wherein each R^{7a} is H.
Embodiment 58. A compound of Formula 1 or any one of Embodiments 1 through 57 wherein independently when an instance of R^{7b} is not taken together with the carbon atom to which the instance of R^{7b} is attached and a geminal instance of R^{7a} to form a carbocyclic ring (i.e. R^{7b} is taken alone) then the instance of R^{7b} is independently H or methyl.
Embodiment 59. A compound of Embodiment 58 wherein each R^{7b} is H.
Embodiment 59a. A compound of Formula 1 or any one of Embodiments 1 through 59 wherein when a pair of R^{7a} and R^{7b} attached to the same carbon atom are taken together with the carbon atom to form a carbocyclic ring, the ring is a cyclopropyl ring.
Embodiment 60. A compound of Formula 1 or any one of Embodiments 1 through 59a wherein each pair of R^{7a} and R^{7b} attached to the same carbon atom are not taken together to form a carbocyclic ring (i.e. R^{7a} and R^{7b} are taken alone).
Embodiment 61. A compound of Formula 1 or any one of Embodiments 1 through 60 wherein each R⁸, R^{9a}, R^{9b}, R¹⁰ and R¹¹ is independently H, C₁-C₂ alkyl, C₂-C₃ alkenyl, C₂-C₃ alkynyl or C₁-C₂ haloalkyl.
Embodiment 62. A compound of Embodiment 61 wherein each R⁸, R^{9a}, R^{9b}, R¹⁰ and R¹¹ is independently H or methyl.
Embodiment 63. A compound of Formula 1 or any one of Embodiments 1 through 62 wherein when J is Q², X is CR², Y is N and Z is CR⁴ and Q¹ is a phenyl ring or a naphthalenyl ring system, each ring or ring system optionally substituted with up to 5 substituents independently selected from R^{5a}; or a 5- to 6-membered fully unsaturated heterocyclic ring or an 8- to 10-membered heteroaromatic bicyclic ring system, each ring or ring system containing ring members selected from carbon atoms and up to 4 heteroatoms independently selected from up to 2 O, up to 2S and up to 4N atoms, wherein up to 3 carbon atom ring members are independently selected from C(═O) and C(═S), and the sulfur atom ring members are independently selected from S(═O)ₚ(═NR⁶)_{f}, each ring or ring system optionally substituted with up to 5 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members, R⁴ is halogen and Q² is a 2-pyridinyl ring substituted with halogen at an ortho position, then said 2-pyridinyl ring is also substituted with R^{5b} at a meta position; provided that when Q² is a phenyl ring substituted with halogen at an ortho position and Q¹ is neither phenyl nor substituted phenyl, then said Q² phenyl ring is also substituted with R^{5b} at least one meta position.
Embodiment 63a. A compound of Formula 1 or any one Embodiments 1 through 62 wherein when J is Q², X is CR², Y is N and Z is CR⁴ and Q¹ is a phenyl ring or a naphthalenyl ring system, each ring or ring system optionally substituted with up to 5 substituents independently selected from R^{5a}; or a 5- to 6-membered fully unsaturated heterocyclic ring or an 8- to 10-membered heteroaromatic bicyclic ring system, each ring or ring system containing ring members selected from carbon atoms and up to 4 heteroatoms independently selected from up to 2 O, up to 2S and up to 4N atoms, wherein up to 3 carbon atom ring members are independently selected from C(═O) and C(═S), and the sulfur atom ring members are independently selected from S(═O)ₚ(═NR⁶)_{f}, each ring or ring system optionally substituted with up to 5 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members, R⁴ is halogen and Q² is a phenyl ring or 2-pyridinyl ring substituted with halogen at an ortho position, then said phenyl ring or 2-pyridinyl ring is also substituted with R^{5b} at a meta position.
Embodiment 64. A compound of Formula 1 or any one Embodiments 1 through 62 wherein when J is Q², X is CR², Y is N and Z is CR⁴ and Q² is a phenyl ring or a naphthalenyl ring system, each ring or ring system optionally substituted with up to 5 substituents independently selected from R^{5b}; or a 5- to 6-membered fully unsaturated heterocyclic ring or an 8- to 10-membered heteroaromatic bicyclic ring system, each ring or ring system containing ring members selected from carbon atoms and up to 4 heteroatoms independently selected from up to 2 O, up to 2S and up to 4N atoms, wherein up to 3 carbon atom ring members are independently selected from C(═O) and C(═S), and the sulfur atom ring members are independently selected from S(═O)ₚ(═NR⁶)_{f}, each ring or ring system optionally substituted with up to 5 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members, R² is halogen and Q¹ is a 2-pyridinyl ring substituted with halogen at an ortho position, then said 2-pyridinyl ring is also substituted with R^{5a} at a meta position; provided that when Q¹ is phenyl ring substituted with halogen at an ortho position and Q² is neither phenyl nor substituted phenyl, then said Q¹ phenyl ring is also substituted with R^{5a} at least one meta position.
Embodiment 64a. A compound of Formula 1 or any one Embodiments 1 through 62 wherein when J is Q², X is CR², Y is N and Z is CR⁴ and Q² is a phenyl ring or a naphthalenyl ring system, each ring or ring system optionally substituted with up to 5 substituents independently selected from R^{5b}; or a 5- to 6-membered fully unsaturated heterocyclic ring or an 8- to 10-membered heteroaromatic bicyclic ring system, each ring or ring system containing ring members selected from carbon atoms and up to 4 heteroatoms independently selected from up to 2 O, up to 2S and up to 4N atoms, wherein up to 3 carbon atom ring members are independently selected from C(═O) and C(═S), and the sulfur atom ring members are independently selected from S(═O)ₚ(═NR⁶)_{f}, each ring or ring system optionally substituted with up to 5 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members and R² is halogen and Q¹ is a phenyl ring or 2-pyridinyl ring substituted with halogen at an ortho position, then said phenyl ring or 2-pyridinyl ring is also substituted with R^{5a} at a meta position;
Embodiment 65. A compound of Formula 1 or any one of Embodiments 1 through 62 wherein when J is Q², X is CR², Y is N and Z is CR⁴, then
   (a) when Q² is an optionally substituted phenyl ring, an optionally substituted naphthalenyl ring system, an optionally substituted fully unsaturated heterocyclic ring, or an optionally substituted heteroaromatic bicyclic ring system, and R² is halogen:
      (i) when Q¹ is a 2-pyridinyl ring substituted with halogen at an ortho position, then said 2-pyridinyl ring is also substituted with R^{5a} at least one meta position, and
      (ii) when Q¹ is phenyl ring substituted with halogen at an ortho position and Q² is neither phenyl nor substituted phenyl, then said Q¹ phenyl ring is also substituted with R^{5a} at least one meta position; and
   (b) when Q¹ is an optionally substituted phenyl ring, an optionally substituted naphthalenyl ring system, an optionally substituted fully unsaturated heterocyclic ring, or an optionally substituted heteroaromatic bicyclic ring system, and R⁴ is halogen:
      (i) when Q² is a 2-pyridinyl ring substituted with halogen at an ortho position, said 2-pyridinyl ring is also substituted with R^{5b} at least one meta position, and
      (ii) when Q² is a phenyl ring substituted with halogen at the ortho position and Q¹ is neither phenyl nor substituted phenyl, then said Q² phenyl ring is also substituted with R^{5b} at least one meta position.
Embodiment 65a. A compound of Formula 1 of Embodiment 65 wherein when R² is halogen and Q¹ is a phenyl ring substituted with halogen at an ortho position, then Q² is neither phenyl nor substituted phenyl; and when R⁴ is halogen and Q² is a phenyl ring substituted with halogen at an ortho position, then Q¹ is neither phenyl nor substituted phenyl.

Embodiments of this invention, including Embodiments 1-65a above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-65a above as well as any other embodiments described herein, and any combination thereof, pertain to the compositions and methods of the present invention.

Combinations of Embodiments 1-65a are illustrated by:
Embodiment A1. A compound of Formula 1 wherein
   Q¹ is a phenyl ring or a naphthalenyl ring system, each ring or ring system optionally substituted with up to 3 substituents independently selected from R^{5a}; or a 5- to 6-membered fully unsaturated heterocyclic ring containing ring members selected from carbon atoms and up to 3 heteroatoms independently selected from up to 2O, up to 2S and up to 3N atoms, wherein up to 2 carbon atom ring members are independently selected from C(═O) and C(═S), and the sulfur atom ring members are independently selected from S(═O)ₛ(═NR⁶)_{f}, the ring optionally substituted with up to 3 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W¹;
   W¹ is a phenyl, thienyl, pyridinyl, pyridazinyl, pyrimidinyl or pyrazolyl ring optionally substituted with up to 3 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members;
   Q² is a phenyl ring or a naphthalenyl ring system, each ring or ring system optionally substituted with up to 3 substituents independently selected from R^{5b}; or a 5- to 6-membered fully unsaturated heterocyclic ring containing ring members selected from carbon atoms and up to 3 heteroatoms independently selected from up to 2O, up to 2S and up to 3N atoms, wherein up to 2 carbon atom ring members are independently selected from C(═O) and C(═S), and the sulfur atom ring members are independently selected from S(═O)ₛ(═NR⁶)_{f}, the ring optionally substituted with up to 3 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W²;
   W² is a phenyl, thienyl, pyridinyl, pyridazinyl, pyrimidinyl or pyrazolyl ring optionally substituted with up to 3 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members;
   Q³ is a phenyl ring or a naphthalenyl ring system, each ring or ring system optionally substituted with up to 3 substituents independently selected from R^{5c}; or a 5- to 6-membered fully unsaturated heterocyclic ring containing ring members selected from carbon atoms and up to 3 heteroatoms independently selected from up to 2O, up to 2S and up to 3N atoms, wherein up to 2 carbon atom ring members are independently selected from C(═O) and C(═S), and the sulfur atom ring members are independently selected from S(═O)ₛ(═NR⁶)_{f}, the ring optionally substituted with up to 3 substituents independently selected from R^{5c} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W³;
   W³ is a phenyl, thienyl, pyridinyl, pyridazinyl, pyrimidinyl or pyrazolyl ring optionally substituted with up to 3 substituents independently selected from R^{5c} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members;
   each R^{7a} is independently H, cyano or methyl;
   each R^{7b} is independently H or methyl; or
   a pair of R^{7a} and R^{7b} attached to the same carbon atom are taken together with the carbon atom to form a cyclopropyl ring;
Embodiment A2. A compound of Embodiment A1 wherein
   Q¹ is a phenyl, thienyl, pyridinyl, pyridazinyl, pyrimidinyl or pyrazolyl ring or a naphthalenyl ring system, each ring or ring system optionally substituted with up to 3 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W¹;
   W¹ is a phenyl or pyridinyl ring optionally substituted with up to 3 substituents independently selected from R^{5a};
   Q² is a phenyl, thienyl, pyridinyl, pyridazinyl, pyrimidinyl or pyrazolyl ring or a naphthalenyl ring system, each ring or ring system optionally substituted with up to 3 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W²;
   W² is a phenyl or pyridinyl ring optionally substituted with up to 3 substituents independently selected from R^{5b};
   Q³ is a phenyl, thienyl, pyridinyl, pyridazinyl, pyrimidinyl or pyrazolyl ring or a naphthalenyl ring system, each ring or ring system optionally substituted with up to 3 substituents independently selected from R^{5c} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W³;
   W³ is a phenyl or pyridinyl ring optionally substituted with up to 3 substituents independently selected from R^{5c};
   each R², R³ and R⁴ is independently H, halogen, C₂-C₃ alkenyl, C₂-C₃ alkynyl, C₂-C₃ haloalkenyl, C₃-C₆ cycloalkyl, C₁-C₃ alkoxy, C₁-C₃ alkylthio, C₁-C₃ alkylamino, C₂-C₄ dialkylamino or C₁-C₃ hydroxyalkyl; or C₁-C₃ alkyl or C₁-C₃ haloalkyl, each optionally substituted with up to 1 substituent independently selected from hydroxy, cyano, C(═O)OR⁸, C(═O)NR^{9a}R^{9b}, C(═O)R¹⁰ and CH═NOR¹¹;
   each R^{5a}, R^{5b} and R^{5c} is independently halogen, cyano, C₁-C₃ alkyl, C₂-C₃ alkenyl, C₂-C₃ alkynyl, C₁-C₃ haloalkyl, C₃ cycloalkyl, C₁-C₃ alkoxy, C₁-C₃ haloalkoxy, C₁-C₃ alkylthio, C₁-C₃ alkylamino, C₂-C₄ dialkylamino C₂-C₄ alkylcarbonyl, C₂-C₄ alkoxycarbonyl, C₂-C₄ alkylcarbonylamino or -U-V-T;
   U is O or NH;
   V is C₂-C₄ alkylene;
   T is NR^{13a}R^{13b} or OR¹⁴;
   each R^{7a} and R^{7b} is independently H or methyl;
   each R⁸, R^{9a}, R^{9b}, R¹⁰ and R¹¹ is independently H or methyl;
   each R^{13a} and R^{13b} is independently H, C₁-C₆ alkyl or C₁-C₆ haloalkyl; and
   each R¹⁴ is independently H, C₁-C₆ alkyl or C₁-C₆ haloalkyl.
Embodiment A3. A compound of Embodiment A2 wherein
   Q¹ is a phenyl or pyridinyl ring optionally substituted with up to 3 substituents independently selected from R^{5a};
   Q² is a phenyl or pyridinyl ring optionally substituted with up to 3 substituents independently selected from R^{5b}; and
   Q³ is a phenyl or pyridinyl ring optionally substituted with up to 3 substituents independently selected from R^{5c}.
Embodiment A4. A compound of Embodiment A3 wherein
   each R², R³ and R⁴ is independently H, halogen, cyano or C₁-C₃ alkyl; and
   each R^{5a}, R^{5b} and R^{5c} is independently halogen, cyano, C₁-C₃ alkyl, C₂-C₃ alkenyl, C₁-C₃ haloalkyl, C₁-C₃ alkoxy, C₁-C₃ alkylthio or C₁-C₃ alkylamino.
Embodiment A5. A compound of Embodiment A4 wherein
   J is Q²;
   X is CR²;
   Y is N;
   Z is CR⁴;
   each R² and R⁴ is independently Cl, Br, I or C₁-C₂ alkyl;
   each R^{5a} and R^{5b} is independently F, Cl, Br, cyano, C₁-C₂ alkyl, C₁-C₂ haloalkyl or C₁-C₂ alkoxy; and
   one of the Q¹ and Q² rings is substituted with 2 to 3 substituents and the other of the Q¹ and Q² rings is substituted with 1 to 2 substituents.

Specific embodiments include compounds of Formula 1 selected from the group consisting of:
4-chloro-1-(4-chlorophenyl)-5-(2,4,6-trifluorophenyl)-1H-imidazole;
4-chloro-1-(4-chlorophenyl)-5-(2,6-difluorophenyl)-2-methyl-1H-imidazole;
2,4-dichloro-1-(4-chlorophenyl)-5-(2,6-difluorophenyl)-1H-imidazole;
1-(4-chlorophenyl)-5-(2,6-difluorophenyl)-2,4-dimethyl-1H-imidazole;
4-chloro-1-(4-chlorophenyl)-2-methyl-5-(2,4,6-trifluorophenyl)-1H-imidazole;
1-(4-chlorophenyl)-2,4-dimethyl-5-(2,4,6-trifluorophenyl)-1H-imidazole;
1-(4-chlorophenyl)-2,4-dichloro-5-(2,4,6-trifluorophenyl)-1H-imidazole;
1-(4-chlorophenyl)-5-(2,6-difluoro-4-methoxyphenyl)-2,4-dimethyl-1H-imidazole;
1-(4-chlorophenyl)-2,4-dichloro-5-(2,6-difluoro-4-methoxyphenyl)-1H-imidazole;
4-chloro-1-(4-chlorophenyl)-5-(2,6-difluoro-4-methoxyphenyl)-2-methyl-1H-imidazole;
4-chloro-1-(4-chloro-3-fluorophenyl)-2-methyl-5-(2,4,6-trifluorophenyl)-1H-imidazole;
1-(4-chloro-3-fluorophenyl)-2,4-dichloro-5-(2,4,6-trifluorophenyl)-1H-imidazole;
1-(4-chloro-3-fluorophenyl)-2,4-dimethyl-5-(2,4,6-trifluorophenyl)-1H-imidazole;
1-(4-chloro-3-fluorophenyl)-5-(2,6-difluorophenyl)-2,4-dimethyl-1H-imidazole;
1-(4-chloro-3-fluorophenyl)-2,4-dichloro-5-(2,6-difluorophenyl)-1H-imidazole;
4-chloro-1-(4-chloro-3-fluorophenyl)-5-(2,6-difluoro-4-methoxyphenyl)-2-methyl-1H-imidazole;
1-(4-chloro-3-fluorophenyl)-5-(2,6-difluoro-4-methoxyphenyl)-2,4-dimethyl-1H-imidazole;
1-(4-chloro-3-fluorophenyl)-2,4-dichloro-5-(2,6-difluoro-4-methoxyphenyl)-1H-imidazole;
4-chloro-1-(3-fluorophenyl)-2-methyl-5-(2,4,6-trifluorophenyl)-1H-imidazole;
2,4-dichloro-1-(3-fluorophenyl)-5-(2,4,6-trifluorophenyl)-1H-imidazole;
4-chloro-1-(3-fluorophenyl)-5-(2,6-difluoro-4-methoxyphenyl)-2-methyl-1H-imidazole;
1-(3-fluorophenyl)-5-(2,6-difluoro-4-methoxyphenyl)-2,4-dimethyl-1H-imidazole;
1-(3-fluorophenyl)-2,4-dichloro-5-(2,6-difluoro-4-methoxyphenyl)-1H-imidazole;
4-chloro-1-(4-chlorophenyl)-2-methyl-5-(2,3,6-trifluorophenyl)-1H-imidazole;
1-(4-chlorophenyl)-2,4-dimethyl-5-(2,3,6-trifluorophenyl)-1H-imidazole;
1-(4-chlorophenyl)-2,4-dichloro-5-(2,3,6-trifluorophenyl)-1H-imidazole;
4-chloro-1-(3-fluorophenyl)-2-methyl-5-(2,3,6-trifluorophenyl)-1H-imidazole;
1-(3-fluorophenyl)-2,4-dimethyl-5-(2,3,6-trifluorophenyl)-1H-imidazole;
1-(3-fluorophenyl)-2,4-dichloro-5-(2,3,6-trifluorophenyl)-1H-imidazole;
4-chloro-5-(2-chloro-4-fluorophenyl)-1-(4-chlorophenyl)-2-methyl-1H-imidazole;
5-(2-chloro-4-fluorophenyl)-1-(4-chlorophenyl)-2,4-dimethyl-1H-imidazole;
5-(2-chloro-4-fluorophenyl)-1-(4-chlorophenyl)-2,4-dichloro-1H-imidazole;
4-chloro-1-(4-chlorophenyl)-5-(2,6-difluoro-3-methoxyphenyl)-2-methyl-1H-imidazole;
4-chloro-5-(2,6-difluoro-4-methoxyphenyl)-1-(4-fluorophenyl)-2-methyl-1H-imidazole;
4-chloro-5-(2,6-difluoro-4-methoxyphenyl)-2-methyl-1-(4-methylphenyl)-1H-imidazole;
4-[4-chloro-1-(4-chlorophenyl)-2-methyl-1H-imidazol-5-yl]-3,5-difluorobenzonitrile;
2-bromo-4-chloro-5-(2,6-difluoro-4-methoxyphenyl)-1-(3-fluorophenyl)-1H-imidazole;
2-chloro-1-(4-chloro-3-fluorophenyl)-5-(2,6-difluoro-4-methoxyphenyl)-4-methyl-1H-imidazole;
2-chloro-5-[2-chloro-5-(2,6-difluoro-4-methoxyphenyl)-4-methyl-1H-imidazol-1-yl]pyridine;
2-bromo-4-chloro-5-(2,6-difluoro-3-methoxyphenyl)-1-(4-fluorophenyl)-1H-imidazole;
4-chloro-5-(2,6-difluoro-3-methoxyphenyl)-2-methyl-1-(4-methylphenyl)-1H-imidazole;
4-chloro-5-(2,6-difluoro-3-methoxyphenyl)-1-(4-fluorophenyl)-2-methyl-1H-imidazole;
2,4-dichloro-5-(2,6-difluoro-3-methoxyphenyl)-1-(4-fluorophenyl)-1H-imidazole;
2,4-dichloro-1-(4-chloro-3-fluorophenyl)-5-(2,6-difluoro-3-methoxyphenyl)-1H-imidazole;
4-chloro-1-(4-chloro-3-fluorophenyl)-5-(2,6-difluoro-3-methoxyphenyl)-2-methyl-1H-imidazole;
2-chloro-5-[2,4-dichloro-5-(2,6-difluoro-4-methoxyphenyl)-1H-imidazol-1-yl]pyridine;
4-chloro-1-[3-(difluoromethoxy)phenyl]-5-(2,6-difluoro-3-methoxyphenyl)-2-methyl-1H-imidazole;
3-[4-chloro-1-(4-chlorophenyl)-2-methyl-1H-imidazol-5-yl]-2,4-difluorobenzonitrile;
4-chloro-5-(2,6-difluoro-4-methoxyphenyl)-1-(3,4-difluorophenyl)-2-methyl-1H-imidazole;
4-chloro-1-(3-chlorophenyl)-5-(2,6-difluoro-4-methoxyphenyl)-2-methyl-1H-imidazole;
4-bromo-2-chloro-1-(4-chlorophenyl)-5-(2,6-difluoro-3-methoxyphenyl)-1H-imidazole;
5-[2,4-dichloro-5-(2,6-difluoro-4-methoxyphenyl)-1H-imidazol-yl]-2-methylpyridine;
5-[2,4-dibromo-5-(2,6-difluoro-4-methoxyphenyl)-1H-imidazol-1-yl]-2-methylpyridine;
2-chloro-5-(4-chlorophenyl)-1-(2,6-difluoro-4-methoxyphenyl)-4-methyl-imidazole;
4-chloro-5-(2,6-difluoro-4-methoxyphenyl)-1-(4-fluorophenyl)-1H-imidazole-2-carboxaldehyde oxime;
4-chloro-1-(2,6-dichlorophenyl)-2-methyl-5-[(2,4,6-trifluorophenyl)methyl]-1H-imidazole;
2-chloro-5-[2,4-dichloro-5-(2,6-difluoro-4-methoxyphenyl)-1H-imidazol-1-yl-4-methyl]pyridine;
4-(2-chloro-4-fluorophenyl)-5-[(2,4-difluorophenyl)methyl]-1,3-dimethyl-1H-pyrazole; and
2-chloro-1-(4-chlorophenyl)-5-(2,6-difluorophenyl)-4-methyl-1H-imidazole;
2-chloro-1-(4-chlorophenyl)-4-methyl-5-(2,4,6-trifluorophenyl)-1H-imidazole;
2-chloro-1-(4-chlorophenyl)-5-(2,6-difluoro-4-methoxyphenyl)-4-methyl-1H-imidazole;
2-chloro-1-(4-chloro-3-fluorophenyl)-4-methyl-5-(2,4,6-trifluorophenyl)-1H-imidazole;
2-chloro-1-(4-chloro-3-fluorophenyl)-5-(2,6-difluoro-4-methoxyphenyl)-4-methyl-1H-imidazole;
2-chloro-1-(3-fluorophenyl)-4-methyl-5-(2,4,6-trifluorophenyl)-1H-imidazole;
2-chloro-1-(3-fluorophenyl)-5-(2,6-difluoro-4-methoxyphenyl)-4-methyl-1H-imidazole;
2-chloro-1-(4-chlorophenyl)-4-methyl-5-(2,3,6-trifluorophenyl)-1H-imidazole;
2-chloro-1-(3-fluorophenyl)-4-methyl-5-(2,3,6-trifluorophenyl)-1H-imidazole;
2-chloro-1-(4-chlorophenyl)-5-(2,6-difluoro-3-methyoxyphenyl)-4-methyl-1H-imidazole;
2-chloro-5-(2,6-difluoro-4-methoxyphenyl)-1-(4-fluorolphenyl)-4-methyl-1H-imidazole;
2-chloro-5-(2,6-difluoro-4-methoxyphenyl)-4-methyl-1-(4-methylphenyl)-1H-imidazole;
4-[2-chloro-1-(4-chlorophenyl)-4-methyl-1H-imidazol-5-yl]-3,5-difluorobenzonitrile;
2-chloro-5-(2,6-difluoro-3-methoxyphenyl)-4-methyl-1-(4-methylphenyl)-1H-imidazole;
2-chloro-5-(2,6-difluoro-3-methoxyphenyl)-1-(4-fluorophenyl)-4-methyl-1H-imidazole;
2-chloro-1-(4-chloro-3-fluorophenyl)-5-(2,6-difluoro-3-methoxyphenyl)-1H-imidazole;
2-chloro-1-[3-(difluoromethoxy)phenyl]-5-(2,6-difluoro-3-methoxyphenyl)-4-methyl-1H-imidazole;
3-[2-chloro-1-(4-chlorophenyl)-4-methyl-1H-imidazol-5-yl]-2,4-difluorobenzonitrile;
2-chloro-5-(2,6-difluoro-4-methoxyphenyl)-1-(3,4-difluorophenyl)-4-methyl-1H-imidazole;
2-chloro-1-(3-chlorophenyl)-5-(2,6-difluoro-4-methoxphenyl)-4-methyl-1H-imidazole;
2-fluoro-5-[2-chloro-5-(2,6-difluoro-4-methoxyphenyl)-4-methyl-1H-imidazol-1-yl]pyridine;
2-bromo-5-[2-chloro-5-(2,6-difluoro-4-methoxyphenyl)-4-methyl-1H-imidazol-1-yl]pyridine;
2-methoxy-5-[2-chloro-5-(2,6-difluoro-4-methoxyphenyl)-4-methyl-1H-imidazol-1-yl]pyridine;
2-trifluoromethoxy-5-[2-chloro-5-(2,6-difluoro-4-methoxyphenyl)-4-methyl-1H-imidazol-1-yl]pyridine;
2-trifluoromethyl-5-[2-chloro-5-(2,6-difluoro-4-methoxyphenyl)-4-methyl-1H-imidazol-1-yl]pyridine;
2-cyano-5-[2-chloro-5-(2,6-difluoro-4-methoxyphenyl)-4-methyl-1H-imidazol-1-yl]pyridine;
3-fluoro-5-[2-chloro-5-(2,6-difluoro-4-methoxyphenyl)-4-methyl-1H-imidazol-1-yl]pyridine;
3-bromo-5-[2-chloro-5-(2,6-difluoro-4-methoxyphenyl)-4-methyl-1H-imidazol-1-yl]pyridine;
3-chloro-5-[2-chloro-5-(2,6-difluoro-4-methoxyphenyl)-4-methyl-1H-imidazol-1-yl]pyridine;
3-methoxy-5-[2-chloro-5-(2,6-difluoro-4-methoxyphenyl)-4-methyl-1H-imidazol-1-yl]pyridine;
1-(4-chlorophenyl)-5-(2,6-difluoro-4-ethoxyphenyl)-2,4-dimethyl-1H-imidazole;
1-(4-fluorophenyl)-5-(2,6-difluoro-4-ethoxyphenyl)-2,4-dimethyl-1H-imidazole;
1-(3-chlorophenyl)-5-(2,6-difluoro-4-ethoxyphenyl)-2,4-dimethyl-1H-imidazole;
1-(3-fluorophenyl)-5-(2,6-difluoro-4-ethoxyphenyl)-2,4-dimethyl-1H-imidazole;
1-(4-methylphenyl)-5-(2,6-difluoro-4-ethoxyphenyl)-2,4-dimethyl-1H-imidazole; and
1-(3-methylphenyl)-5-(2,6-difluoro-4-ethoxyphenyl)-2,4-dimethyl-1H-imidazole.

Another aspect of the present invention relates to compounds of Formula 1P (including all geometric and stereoisomers), N-oxides, and salts thereof, agricultural compositions containing them and their use as fungicides:

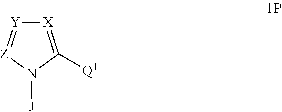

wherein
J is Q² or R¹;
X is N, CR² or CQ³;
Y is N or CR³;
Z is N or CR⁴;
Q¹ is a phenyl, thienyl, pyridinyl, pyridazinyl, pyrimidinyl or pyrazolyl ring, or a naphthalenyl ring system, each ring or ring system optionally substituted with up to 5 substituents independently selected from R^{5a};
Q² is a phenyl, thienyl, pyridinyl, pyridazinyl, pyrimidinyl or pyrazolyl ring, or a naphthalenyl ring system, each ring or ring system optionally substituted with up to 5 substituents independently selected from R^{5b};
Q³ is a phenyl, thienyl, pyridinyl, pyridazinyl, pyrimidinyl or pyrazolyl ring, or a naphthalenyl ring system, each ring or ring system optionally substituted with up to 5 substituents independently selected from R^{5c};
R¹ is C₁-C₇ alkyl, C₂-C₇ alkenyl, C₂-C₇ alkynyl, C₃-C₇ cycloalkyl, C₁-C₇ haloalkyl, C₂-C₇ haloalkenyl, C₄-C₁₀ alkylcycloalkyl or C₄-C₁₀ cycloalkylalkyl;
each R², R³ and R⁴ is independently H, halogen, cyano, nitro, C₁-C₇ alkyl, C₂-C₇ alkenyl, C₇-C₇ alkynyl, C₃-C₇ cycloalkyl, C₁-C₇ haloalkyl, C₂-C₇ haloalkenyl, C₁-C₇ alkoxy, C₁-C₇ haloalkoxy, C₁-C₇ alkylthio, C₁-C₇ alkylsulfinyl, C₁-C₇ alkylsulfonyl, C₁-C₇ haloalkylthio, C₁-C₇ haloalkylsulfinyl, C₁-C₇ haloalkylsulfonyl, C₁-C₇ alkylamino or C₂-C₇ dialkylamino; and
each R^{5a}, R^{5b} and R^{5c} is independently halogen, cyano, nitro, C₁-C₇ alkyl, C₂-C₇ alkenyl, C₂-C₇ alkynyl, C₃-C₇ cycloalkyl, C₁-C₇ haloalkyl, C₂-C₇ haloalkenyl, C₁-C₇ alkoxy, C₁-C₆ alkylthio, C₁-C₆ haloalkylthio, C₁-C₇ haloalkoxy, C₁-C₇ haloalkylthio, C₁-C₇ haloalkylsulfinyl, C₁-C₇ haloalkylsulfonyl, C₁-C₇ alkylamino, C₂-C₇ dialkylamino, C₂-C₇ alkylcarbonyl, C₂-C₇ alkoxycarbonyl or C₃-C₁₀ trialkylsilyl;

provided that:
(a) when J is R¹, then X is CQ³;
(b) when J is Q², then X is N or CR², and if X is N or CH, then Z is other than CH; and
(c) when Q¹ is a phenyl ring which is not substituted by R^{5a} at either ortho positions, then when X is N or CR² and Q² is a phenyl ring, the Q² phenyl ring is substituted by at least one R^{5b} at an ortho position; and when X is CQ³ and Q³ is a phenyl ring, the Q³ phenyl ring is substituted by at least one R^{5c} at an ortho position.

One skilled in the art recognizes that the definition of substituents on Formula 1P overlap the definition of substituents on Formula 1 as described in the Summary of the Invention and therefore disclosure herein relative to Formula 1 also extends to Formula 1P.

More particularly, this aspect of the present invention pertains to a compound of Formula 1P (including all geometric and stereoisomers), an N-oxide or a salt thereof.

Related to this aspect is a fungicidal composition comprising (a) a compound selected from Formula 1P, N-oxides and salts thereof; and (b) at least one other fungicide.

Also related to this aspect is a fungicidal composition comprising (a) a fungicidally effective amount of a compound selected from Formula 1P, N-oxides and salts thereof; and (b) at least one additional component selected from the group consisting of surfactants, solid diluents and liquid diluents.

Also related to this aspect is a method for controlling plant diseases caused by fungal plant pathogens comprising applying to the plant or portion thereof, or to the plant seed, a fungicidally effective amount of a compound selected from Formula 1P, N-oxides and salts thereof (e.g., as a composition described herein).

Embodiments of this aspect include Embodiments B1 through B53 described below. In the following Embodiments, Formula 1P includes N-oxides and salts thereof, and reference to “a compound of Formula 1P” includes the definitions of substituents specified above for Formula 1P unless further defined in the Embodiments.
Embodiment B1. A compound of Formula 1P wherein J is Q².
Embodiment B2. A compound of Formula 1P wherein J is R¹.
Embodiment B3. A compound of Formula 1P or Embodiment B1 wherein R¹ is C₁-C₂ alkyl.
Embodiment B4. A compound of Formula 1P wherein X is CR².
Embodiment B5. A compound of Formula 1P wherein X is CQ³.
Embodiment B6. A compound of Formula 1P wherein Y is N.
Embodiment B7. A compound of Formula 1P wherein Y is CR³.
Embodiment B8. A compound of Formula 1P wherein Z is CR⁴.
Embodiment B9. A compound of Formula 1P wherein Z is N.
Embodiment B10. A compound of Formula 1P wherein Q¹ is a phenyl, thienyl, pyridinyl, pyridazinyl, pyrimidinyl or pyrazolyl ring, or a naphthalenyl ring system, each ring or ring system optionally substituted with up to 3 substituents independently selected from R^{5a}.
Embodiment B 11. A compound of Formula 1P wherein Q² is a phenyl, thienyl, pyridinyl, pyridazinyl, pyrimidinyl or pyrazolyl ring, or a naphthalenyl ring system, each ring or ring system optionally substituted with up to 3 substituents independently selected from R^{5b}.
Embodiment B12. A compound of Formula 1P wherein Q³ is a phenyl, thienyl, pyridinyl, pyridazinyl, pyrimidinyl or pyrazolyl ring, or a naphthalenyl ring system, each ring or ring system optionally substituted with up to 3 substituents independently selected from R^{5c}.
Embodiment B13. A compound of Embodiment B10 wherein Q¹ is a phenyl ring optionally substituted with up to 3 substituents independently selected from R^{5a}.
Embodiment B14. A compound of Embodiment B11 wherein Q² is a phenyl ring optionally substituted with up to 3 substituents independently selected from R^{5b}
Embodiment B15. A compound of Embodiment B12 wherein Q³ is a phenyl ring optionally substituted with up to 3 substituents independently selected from R^{5c}.
Embodiment B16. A compound of Embodiment B13 wherein Q¹ is a phenyl ring optionally substituted with up to 2 substituents independently selected from R^{5a}.
Embodiment B17. A compound of Embodiment B14 wherein Q² is a phenyl ring optionally substituted with up to 2 substituents independently selected from R^{5b}.
Embodiment B18. A compound of Embodiment B15 wherein Q³ is a phenyl ring optionally substituted with up to 2 substituents independently selected from R^{5c}.
Embodiment B19. A compound of Embodiment B16 wherein Q¹ is a phenyl ring optionally substituted with up to 1 substituent selected from R^{5a}.
Embodiment B20. A compound of Embodiment B17 wherein Q² is a phenyl ring optionally substituted with up to 1 substituent selected from R^{5b}.
Embodiment B21. A compound of Embodiment B18 wherein Q³ is a phenyl ring optionally substituted with up to 1 substituent selected from R^{5c}.
Embodiment B22. A compound of Formula 1P or Embodiment B13 wherein Q¹ is a phenyl ring substituted with up to 3 substituents independently selected from R^{5a} attached at ortho and/or para positions.
Embodiment B23. A compound of Formula 1P or Embodiment B14 wherein Q² is a phenyl ring substituted with up to 3 substituents independently selected from R^{5b} attached at ortho and/or para positions.
Embodiment B24. A compound of Formula 1P or Embodiment B15 wherein Q³ is a phenyl ring substituted with up to 3 substituents independently selected from R⁵ attached at ortho and/or para positions.
Embodiment B25. A compound of Formula 1P or Embodiment B22 wherein Q¹ is a phenyl ring substituted with 3 substituents independently selected from R^{5a} attached at ortho and para positions.
Embodiment B26. A compound of Formula 1P or Embodiment B23 wherein Q² is a phenyl ring substituted with 3 substituents independently selected from R^{5b} attached at ortho and para positions.
Embodiment B27. A compound of Formula 1P or Embodiment B24 wherein Q³ is a phenyl ring substituted with 3 substituents independently selected from R⁵ attached at ortho and para positions.
Embodiment B28. A compound of Formula 1P or Embodiment B22 wherein Q¹ is a phenyl ring substituted with up to 2 substituents independently selected from R^{5a} attached at ortho and/or para positions.
Embodiment B29. A compound of Formula 1P or Embodiment B23 wherein Q² is a phenyl ring substituted with up to 2 substituents independently selected from R^{5b} attached at ortho and/or para positions.
Embodiment B30. A compound of Formula 1P or Embodiment B24 wherein Q³ is a phenyl ring substituted with up to 2 substituents independently selected R⁵ attached at ortho and/or para positions.
Embodiment B31. A compound of Formula 1P or Embodiment B28 wherein Q¹ is a phenyl ring substituted with 2 substituents independently selected from R^{5a} attached at one ortho position and the para position.
Embodiment B32. A compound of Formula 1P or Embodiment B29 wherein Q² is a phenyl ring substituted with 2 substituents independently selected from R^{5b} attached at one ortho position and the para position.
Embodiment B33. A compound of Formula 1P or Embodiment B30 wherein Q³ is a phenyl ring substituted with 2 substituents independently selected from R⁵ attached at one ortho position and the para position.
Embodiment B34. A compound of Formula 1P or Embodiment B28 wherein Q¹ is a phenyl ring substituted with 2 substituents independently selected from R^{5a} attached at ortho positions.
Embodiment B35. A compound of Formula 1P or Embodiment B29 wherein Q² is a phenyl ring substituted with 2 substituents independently selected from R^{5b} attached at ortho positions.
Embodiment B36. A compound of Formula 1P or Embodiment B30 wherein Q³ is a phenyl ring substituted with 2 substituents independently selected from R⁵ attached at ortho positions.
Embodiment B37. A compound of Formula 1P wherein Q¹ is a phenyl ring substituted with 2 substituents independently selected from R^{5a} attached at one meta position and the para position.
Embodiment B38. A compound of Formula 1P wherein Q² is a phenyl ring substituted with 2 substituents independently selected from R^{5b} attached at one meta position and the para position.
Embodiment B39. A compound of Formula 1P wherein Q³ is a phenyl ring substituted with 2 substituents independently selected from R^{5c} attached at one meta position and the para position.
Embodiment B40. A compound of Formula 1P or Embodiment B19 wherein Q¹ is a phenyl ring substituted with up to 1 substituent selected from R^{5a} attached at the para position.
Embodiment B41. A compound of Formula 1P or Embodiment B21 wherein Q³ is a phenyl ring substituted with up to 1 substituent selected from R^{5c} attached at the para position.
Embodiment B42. A compound of Formula 1P or Embodiment B40 wherein Q¹ is a phenyl ring substituted with 1 substituent selected from R^{5a} attached at the para position.
Embodiment B43. A compound of Formula 1P wherein Q² is a phenyl ring substituted with 1 substituent selected from R^{5b} attached at the para position.
Embodiment B44. A compound of Formula 1P or Embodiment B41 wherein Q³ is a phenyl ring substituted with 1 substituent selected from R^{5c} attached at the para position.
Embodiment B45. A compound of Formula 1P wherein each R², R³ and R⁴ is independently H, halogen, cyano, C₁-C₃ alkyl, C₂-C₃ alkenyl, C₃ cycloalkyl or C₁-C₃ haloalkyl.
Embodiment B46. A compound of Embodiment B45 wherein each R², R³ and R⁴ is independently H, halogen or C₁-C₃ alkyl.
Embodiment B47. A compound of Embodiment B46 wherein each R², R³ and R⁴ is independently H, Cl, Br, I or C₁-C₂ alkyl.
Embodiment B48. A compound of Embodiment B47 wherein R² is selected from H, Cl, Br, I and C₁-C₂ alkyl.
Embodiment B49. A compound of Embodiment B47 wherein R³ is selected from H, Cl, Br or CH₃;
Embodiment B50. A compound of Embodiment B47 wherein R⁴ is selected from H, Cl, Br or CH₃.
Embodiment B51. A compound of Formula 1P wherein each R^{5a}, R^{5b} and R^{5c} is independently halogen, cyano, nitro, C₁-C₃ alkyl, C₂-C₃ alkenyl, C₃ cycloalkyl, C₁-C₃ haloalkyl, C₂-C₃ haloalkenyl, C₁-C₃ alkoxy, C₁-C₃ alkylthio, C₁-C₃ haloalkylthio, C₁-C₃ haloalkoxy or C₁-C₆ haloalkylthio.
Embodiment B52. A compound of Embodiment B51 wherein each R^{5a}, R^{5b} and R^{5c} is independently halogen, cyano, C₁-C₃ alkyl, C₁-C₃ haloalkyl or C₁-C₃ alkoxy.
Embodiment B53. A compound of Embodiment B52 wherein each R^{5a}, R^{5b} and R^{5c} is independently halogen, CH₃, OCH₃ or CF₃.

Embodiments B1-B53 above as well as any other embodiments described herein relevant to Formula 1P, can be combined in any manner, and the descriptions of variables in the embodiments pertain not only to the compounds of Formula 1P but also to the starting compounds and intermediate compounds useful for preparing the compounds of Formula 1P. In addition, Embodiments B1-B53 above as well as any other embodiments described herein relevant to Formula 1P, and any combination thereof, pertain to the compositions and methods relating to compounds of Formula 1P. Of note are counterparts of Embodiments 1-62 wherein “Formula 1” is replaced by “Formula 1P” to the extent that these counterpart embodiments limit the definition of substituents on Formula 1P. Also of note are counterparts of Embodiments B1-B53 wherein “Formula 1P” is replaced by “Formula 1” to the extent that these counterpart embodiments limit the definition of substituents on Formula 1.

Combinations of Embodiments B1-B53 are illustrated by:
Embodiment C1. A compound of Formula 1P wherein
   Q¹ is independently a phenyl, thienyl, pyridinyl, pyridazinyl, pyrimidinyl or pyrazolyl ring, or a naphthalenyl ring system, each ring or ring system optionally substituted with up to 3 substituents independently selected from R^{5a};
   Q² is independently a phenyl, thienyl, pyridinyl, pyridazinyl, pyrimidinyl or pyrazolyl ring, or a naphthalenyl ring system, each ring or ring system optionally substituted with up to 3 substituents independently selected from R^{5b};
   Q³ is independently a phenyl, thienyl, pyridinyl, pyridazinyl, pyrimidinyl or pyrazolyl ring, or a naphthalenyl ring system, each ring or ring system optionally substituted with up to 3 substituents independently selected from R⁵;
   R¹ is C₁-C₇ alkyl, C₂-C₇ alkenyl, C₂-C₇ alkynyl, C₃-C₇ cycloalkyl, C₁-C₇ haloalkyl, C₂-C₇ haloalkenyl, C₄-C₁₀ alkylcycloalkyl or C₄-C₁₀ cycloalkylalkyl;
   each R², R³ and R⁴ is independently H, halogen, cyano, nitro, C₁-C₇ alkyl, C₂-C₇ alkenyl, C₇-C₇ alkynyl, C₃-C₇ cycloalkyl, C₁-C₇ haloalkyl, C₂-C₇ haloalkenyl, C₁-C₇ alkoxy, C₁-C₇ haloalkoxy, C₁-C₇ alkylthio, C₁-C₇ alkylsulfinyl, C₁-C₇ alkylsulfonyl, C₁-C₇ haloalkylthio, C₁-C₇ haloalkylsulfinyl, C₁-C₇ haloalkylsulfonyl, C₁-C₇ alkylamino or C₂-C₇ dialkylamino; and
   each R^{5a}, R^{5b} and R^{5c} is independently halogen, cyano, nitro, C₁-C₇ alkyl, C₂-C₇ alkenyl, C₂-C₇ alkynyl, C₃-C₇ cycloalkyl, C₁-C₇ haloalkyl, C₂-C₇ haloalkenyl, C₁-C₇ alkoxy, C₁-C₆ alkylthio, C₁-C₆ haloalkylthio, C₁-C₇ haloalkoxy, C₁-C₇ haloalkylthio, C₁-C₇ haloalkylsulfinyl, C₁-C₇ haloalkylsulfonyl, C₁-C₇ alkylamino, C₂-C₇ dialkylamino, C₂-C₇ alkylcarbonyl, C₂-C₇ alkoxycarbonyl or C₃-C₁₀ trialkylsilyl.
Embodiment C2. A compound of Formula C1 wherein
   J is Q²;
   X is CR²;
   Y is N; and
   Z is CR⁴.
Embodiment C3. A compound of Embodiment C2 wherein
   each R² and R⁴ is independently H, halogen, cyano, C₁-C₃ alkyl, C₂-C₃ alkenyl, C₃ cycloalkyl or C₁-C₃ haloalkyl; and
   each R^{5a} and R^{5b} is independently halogen, cyano, nitro, C₁-C₃ alkyl, C₂-C₃ alkenyl, C₃ cycloalkyl, C₁-C₃ haloalkyl, C₂-C₃ haloalkenyl, C₁-C₃ alkoxy, C₁-C₃ alkylthio, C₁-C₃ haloalkylthio, C₁-C₃ haloalkoxy or C₁-C₇ haloalkylthio.
Embodiment C4. A compound of Embodiment C3 wherein
   Q¹ is a phenyl ring optionally substituted with up to 3 substituents independently selected from R^{5a};
   Q² is a phenyl ring optionally substituted with up to 3 substituents independently selected from R^{5b};
   each R² and R⁴ is independently H, halogen or C₁-C₃ alkyl; and
   each R^{5a} and R^{5b} is independently halogen, cyano, C₁-C₃ alkyl, C₁-C₃ haloalkyl or C₁-C₃ alkoxy.
Embodiment C5. A compound of Embodiment C4 wherein
   each R² and R⁴ is independently H, Cl, Br, I or C₁-C₂ alkyl; and
   each R^{5a} and R^{5b} is independently halogen, CH₃, OCH₃ or CF₃.
Embodiment C7. A compound of Embodiment Cl wherein
   X is CQ³;
   Y is CR³;
   R³ is H, Cl, Br or CH₃;
   Z is N;
   J is R¹; and
   R¹ is C₁-C₂ alkyl.

Specific embodiments include compounds of Formula 1P selected from the group consisting of:
4-chloro-1-(4-chlorophenyl)-5-(2,4,6-trifluorophenyl)-1H-imidazole;
4-chloro-1-(4-chlorophenyl)-5-(2,6-difluorophenyl)-2-methyl-1H-imidazole;
2,4-dichloro-1-(4-chlorophenyl)-5-(2,6-difluorophenyl)-1H-imidazole; and
2,4-dichloro-1-(4-chlorophenyl)-5-(2,4,6-trifluorophenyl)-1H-imidazole.

Also, specific embodiments include compounds of Formula 1P selected from the group consisting of:
4-chloro-1-(4-chlorophenyl)-5-(2,4,6-trifluorophenyl)-1H-imidazole;
4-chloro-1-(4-chlorophenyl)-5-(2,6-difluorophenyl)-2-methyl-1H-imidazole;
2,4-dichloro-1-(4-chlorophenyl)-5-(2,6-difluorophenyl)-1H-imidazole;
1-(4-chlorophenyl)-2,4-dichloro-5-(2,4,6-trifluorophenyl)-1H-imidazole (or 2,4-dichloro-1-(4-chlorophenyl)-5-(2,4,6-trifluorophenyl)-1H-imidazole);
1-(4-chlorophenyl)-5-(2,6-difluorophenyl)-2,4-dimethyl-1H-imidazole (or 1-(4-chlorophenyl)-5-(2,6-difluorophenyl)-2,4-dimethyl)-1H-imidazole);
4-chloro-1-(4-chlorophenyl)-2-methyl-5-(2,4,6-trifluorophenyl)-1H-imidazole;
1-(4-chlorophenyl)-2,4-dimethyl-5-(2,4,6-trifluorophenyl)-1H-imidazole;
1-(4-chlorophenyl)-2,4-dichloro-5-(2,4,6-trifluorophenyl)-1H-imidazole;
1-(4-chlorophenyl)-5-(2,6-difluoro-4-methoxyphenyl)-2,4-dimethyl-1H-imidazole;
1-(4-chlorophenyl)-2,4-dichloro-5-(2,6-difluoro-4-methoxyphenyl)-1H-imidazole;
4-chloro-1-(4-chlorophenyl)-5-(2,6-difluoro-4-methoxyphenyl)-2-methyl-1H-imidazole;
4-chloro-1-(4-chloro-3-fluorophenyl)-2-methyl-5-(2,4,6-trifluorophenyl)-1H-imidazole;
1-(4-chloro-3-fluorophenyl)-2,4-dichloro-5-(2,4,6-trifluorophenyl)-1H-imidazole;
1-(4-chloro-3-fluorophenyl)-2,4-dimethyl-5-(2,4,6-trifluorophenyl)-1H-imidazole;
1-(4-chloro-3-fluorophenyl)-5-(2,6-difluorophenyl)-2,4-dimethyl-1H-imidazole;
1-(4-chloro-3-fluorophenyl)-2,4-dichloro-5-(2,6-difluorophenyl)-1H-imidazole;
1-(4-chloro-3-fluorophenyl)-5-(2,6-difluorophenyl)-2,4-dimethyl-1H-imidazole;
4-chloro-1-(4-chloro-3-fluorophenyl)-5-(2,6-difluoro-4-methoxyphenyl)-2-methyl-1H-imidazole;
1-(4-chloro-3-fluorophenyl)-5-(2,6-difluoro-4-methoxyphenyl)-2,4-dimethyl-1H-imidazole;
1-(4-chloro-3-fluorophenyl)-2,4-dichloro-5-(2,6-difluoro-4-methoxyphenyl)-1H-imidazole (or 1-(4-chloro-3-fluorophenyl)-2,4-dichloro-5-(2,6-difluoro-4-methoxyphenyl-1H-imidazole);
4-chloro-1-(3-fluorophenyl)-2-methyl-5-(2,4,6-trifluorophenyl)-1H-imidazole;
2,4-dichloro-1-(3-fluorophenyl)-5-(2,4,6-trifluorophenyl)-1H-imidazole;
1-(3-fluorophenyl)-5-(2,4,6-trifluorophenyl)-2,4-dimethyl-1H-imidazole (or 1-(3-fluorophenyl)-5-(2,4,6-trifluorophenyl)-2,4-dimethyl)-1H-imidazole);
4-chloro-1-(3-fluorophenyl)-5-(2,6-difluoro-4-methoxyphenyl)-2-methyl-1H-imidazole;
1-(4-fluorophenyl)-5-(2,6-difluoro-4-methoxyphenyl)-2,4-dimethyl-1H-imidazole (or 1-(4-3-fluorophenyl)-5-(2,6-difluoro-4-methoxyphenyl)-2,4-dimethyl-1H-imidazole);
1-(3-fluorophenyl)-2,4-dichloro-5-(2,6-difluoro-4-methoxyphenyl)-1H-imidazole;
1-(3-fluorophenyl)-2,4-dichloro-5-(2,6-difluoro-4-methoxyphenyl)-1H-imidazole;
1-(3-fluorophenyl)-2,4-dimethyl-5-(2,6-difluoro-4-methoxyphenyl)-1H-imidazole;
4-chloro-1-(3-fluorophenyl)-5-(2,6-difluoro-4-methoxyphenyl)-2-methyl-1H-imidazole;
4-chloro-1-(4-chlorophenyl)-2-methyl-5-(2,3,6-trifluorophenyl)-1H-imidazole;
1-(4-chlorophenyl)-2,4-dimethyl-5-(2,3,6-trifluorophenyl)-1H-imidazole; and
1-(4-chlorophenyl)-2,4-dichloro-5-(2,3,6-trifluorophenyl)-1H-imidazole;
4-chloro-1-(3-fluorophenyl)-2-methyl-5-(2,3,6-trifluorophenyl)-1H-imidazole;
1-(3-fluorophenyl)-2,4-dimethyl-5-(2,3,6-trifluorophenyl)-1H-imidazole;
1-(3-fluorophenyl)-2,4-dichloro-5-(2,3,6-trifluorophenyl)-1H-imidazole;
4-chloro-5-(2-chloro-4-fluorophenyl)-1-(4-chlorophenyl)-2-methyl-1H-imidazole;
5-(2-chloro-4-fluorophenyl)-1-(4-chlorophenyl)-2,4-dimethyl-1H-imidazole; and
5-(2-chloro-4-fluorophenyl)-1-(4-chlorophenyl)-2,4-dichloro-1H-imidazole (or 1-(2-chloro-4-fluorophenyl)-1-(4-chlorophenyl)-2,4-dichloro-1H-imidazol).

Of note are compounds of Formula 1 or Formula 1P including geometric and stereoisomers, N-oxides, and salts thereof (including but not limited to Embodiments 1-62, A1-A5, B1-53 and C1-C6, above) wherein when Q¹ is a phenyl ring which is not substituted by R^{5a} at either ortho positions, then when X is N or CR² and Q² is a phenyl ring, the Q² phenyl ring is substituted by at least one R^{5b} at an ortho position.

This invention provides a fungicidal composition comprising a compound of Formula 1 or Formula 1P (including all geometric and stereoisomers, N-oxides, and salts thereof), and at least one other fungicide. Of note as embodiments of such compositions are compositions comprising a compound corresponding to any of the compound embodiments described above.

This invention provides a fungicidal composition comprising a fungicidally effective amount of a compound of Formula 1 or Formula 1P (including all geometric and stereoisomers, N-oxides, and salts thereof), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound of Formula 1 or Formula 1P (including all geometric and stereoisomers, N-oxides, and salts thereof). Of note as embodiment of such methods are methods comprising applying a fungicidally effective amount of a compound corresponding to any of the compound embodiments describe above. Of particular note are embodiments where the compounds are applied as compositions of this invention.

One or more of the following methods and variations as described in Schemes 1-17 can be used to prepare the compounds of Formula 1 or Formula 1P. The definitions of Q¹, Q², Q³, R¹, R², R³ and R⁴ in the compounds of Formulae 1-26 below are as defined above in the Summary of the Invention unless otherwise noted. Compounds of Formulae 1a-1e are various subsets of Formula 1, and all substituents for Formulae 1a-1e are as defined above for Formula 1 unless otherwise noted.

Compounds of Formula 1a (Formula 1 wherein J is Q², X is CR², Y is N and Z is CR⁴) can be prepared by halogenation or alkylation of compounds of Formula 1b (i.e. Formula 1 wherein J is Q², X is CR², R² is H, Y is N and Z is CR⁴) as illustrated in Scheme 1. Typically halogenation can be achieved using a variety of halogenating reagents known in the art such as elemental halogen (e.g., Cl₂, Br₂, I₂), sulfuryl chloride, iodine monochloride or a N-halosuccinimide (e.g., NBS, NCS, NIS) in an appropriate solvent such as N,N-dimethylformamide, carbon tetrachloride, acetonitrile, dichloromethane or acetic acid. Alkylation is achieved by reacting a compound of Formula 1b with a metalating agent, followed by an alkylating agent of formula R²-Lg (wherein Lg is a leaving group such as Cl, Br, I or a sulfonate, for example, p-toluenesulfonate, methanesulfonate or trifluoromethanesulfonate). Suitable metalating agents include, for example, as n-butyl lithium (n-BuLi), lithium diisopropylamide (LDA) or sodium hydride (NaH). As used herein, the terms “alkylation” and “alkylating agent” are not limited to R² being an alkyl group, and include in addition to alkyl such groups as alkylthio, haloalkyl, alkenyl, haloalkenyl, alkynyl, and the like. For reaction conditions see Almansa et al., Journal of Medicinal Chemistry 2003, 46(16), 3463-3475 and Katritzky et al., Heterocycles 1997, 44, 67-70. Also, the method of Scheme 1 is illustrated in Examples 5, 6 and 8.

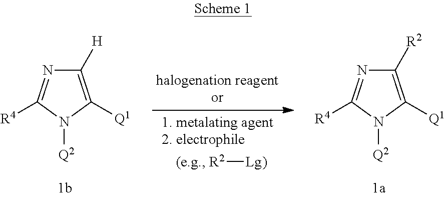

Compounds of Formula 1a can be subjected to various nucleophilic and metallation reactions to add substituents or modify existing substituents, and thus provide other functionalized compounds of Formula 1a. For example, compounds of Formula 1a wherein R² and/or R⁴ are halogen can undergo nucleophilic displacements to provide compounds of Formula 1a wherein R² and/or R⁴ are groups linked to the imidazole ring through an O, S or N atom (e.g., displacements with alkoxides, thiolates and amines). Typically these reactions are run in the presence of a suitable base (e.g., an sodium hydride, potassium t-butoxide, potassium carbonate or triethylamine) in a solvent such as alcohol, acetonitrile or N,N-dimethylformamide at temperatures ranging from room temperature to the reflux temperature of the solvent. General procedures for conducting nucleophilic displacements of halogens are known in the art and can be readily adapted to prepare compounds of the present invention. For procedures relevant to imidazoles see Grimmett et al., Australian Journal of Chemistry 1987, 40(8), 1399-1413.

Additionally, compounds of Formula 1a wherein R² and/or R⁴ are iodo can be used to prepare the corresponding thiocyanate (—SCN) derivatives of Formula 1a. Typical conditions involve contacting the iodo compound of Formula 1a with a thiocyanating agent such as K[Cu(SCN)₂], which is generated in situ from equimolar amounts of copper(I) thiocyanate and potassium thiocyanate. The reaction is typically carried out in a polar solvent such as N,N-dimethylformamide, dimethylacetamide, 1,4-dioxane or dimethylsulfoxide at a temperature between about room temperature and the reflux temperature of the solvent. The reaction can also be carried out at higher temperatures using a microwave reactor. For a reference see, for example, Suzuki et al., Synthetic Communications 1996, 26(18), 3413-3419.

Also, compounds of Formula 1a wherein R² and/or R⁴ are bromo or iodo can be cross-coupled with compounds of formulae R²-Met or R⁴-Met (wherein Met is Sn, Zn, B(OH)₂, Mg, Li, Cu or other suitable counterions) in the presence of a palladium or nickel catalyst to produce compounds of Formula 1a wherein R² and/or R⁴ are cyano, alkyl, alkenyl, haloalkenyl, alkynyl, and the like. Preferred catalysts include but are not limited to Pd(PPh₃)₄, PdCl₂(PPh₃)₂, PdCl₂(diphenylphosphinoferrocene), NiCl₂(PPh₃)₂ and tetrakis(tri-2-furylphosphino)palladium. The exact conditions for each reaction will depend upon the catalyst used and the counterions in the compound of formulae R²-Met or R⁴-Met. The presence of a base (such as an alkali carbonate, tertiary amine or alkali fluoride) is necessary for reactions involving compounds of formulae R²-Met or R⁴-Met where Met is B(OH)₂. Examples 13, 16, 17, 18, 19 and 31 illustrate various cross-coupling reactions for the preparation of certain compounds of Formula 1a.

As shown in Scheme 2, compounds of Formula 1a can alternatively be prepared by halogenation of a compound of Formula 2 preferentially at the 4-position of the imidazole ring to provide a compound of Formula 1c (Formula 1 wherein J is Q², X is CR², Y is N, Z is CR⁴ and R⁴ is H) wherein R² is halogen, which can then be treated with a second equivalent of the same or different halogenating reagent to provide a compound of Formula 1a wherein R² and R⁴ are halogen. For an example illustrating the method of Scheme 2 for the preparation of a compound of Formula Ic see Step C of Examples 1 and 34. Also, for an example illustrating the method of Scheme 2 for the preparation of a compound of Formula 1a wherein R² is chloro and R⁴ is bromo see Example 35. Alternatively, compounds of Formula 2 can be treated with 2 equivalents of a halogenating reagent to provide a compound of Formula 1a directly wherein R² and R⁴ are both the same halogen. For an example illustrating the method of preparing a compound of Formula 1a wherein R² and R⁴ are both the same halogen see Example 2 and Step C of Example 3.

Alternatively as also shown in Scheme 2, compounds of Formula 1a wherein R⁴ is halogen, alkyl, alkylthio, haloalkyl, alkenyl, haloalkenyl, alkynyl, and the like can be prepared from compounds of Formula 1c by metallation with a reagent such as n-butyllithium (n-BuLi), lithium diisopropylamide (LDA) or sodium hydride (NaH) in a solvent such as tetrahydrofuran, dioxane or toluene at temperatures ranging from about 0° C. to room temperature. The anion is then contacted with an electrophile resulting in the introduction of an R⁴ group onto Formula 1c, thus providing a compound of Formula 1a.

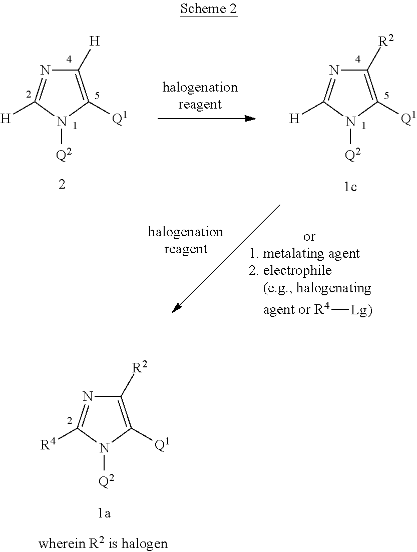

Synthesis of compounds of Formula 1b can be achieved as outlined in Scheme 3. In the first step a compound of Formula 3 is N-arylated with halides of formula Q²X¹ wherein X¹ is I, Cl, Br or F. There are a number of conditions published in the chemistry literature which can be used for introduction of a substituted aryl or a heteroaryl group onto Formula 3, including copper-catalyzed conditions involving the use of a suitable copper source (e.g., copper(I) iodide or copper(I) triflate) and a metal carbonate base (e.g., potassium or cesium carbonate) in a suitable solvent such as xylenes, dioxane or acetonitrile (see Buchwald et al., Tetrahedron Letters 1999, 40, 2657-2660 and Jiang et al., Journal of Organic Chemistry 2007, 72, 8943-8946). The method of Scheme 3 for the preparation of a compound of Formula 4 is also illustrated in Step A of Example 7.

In a subsequent step, compounds of Formula 4 can be converted directly to Formula 1b by reaction with a halide of formula Q¹X¹ in the presence of palladium(II) acetate and a triarylphosphine ligand and cesium fluoride in a solvent such as dioxane, tetrahydrofuran or acetonitrile at the reflux temperature of the solvent. For a representative reference see Bellina et al., Journal of Organic Chemistry 2005, 70, 3997-4005. Also, Step B of Example 7 illustrates the preparation of a compound of Formula 1b using the method of Scheme 3. Alternatively, lithiation of a compound of Formula 4 with n-butyllithium (n-BuLi) or lithium diisopropylamide (LDA), followed by treatment of the anion with trialkylorganostannyl chlorides or boronic acids (or esters) provides compounds of Formula 5. Treatment of compounds Formula 5 with a halide of formula Q¹X¹ using well-known transition metal-catalyzed cross coupling reaction conditions provides Formula 1b compounds. Typically the reaction is run in the present of a palladium catalyst. A wide variety of palladium-containing compounds and complexes are useful as catalysts in the method of Scheme 3, including PdCl₂(PPh₃)₂ (bis(triphenylphosphine)palladium (II) dichloride), Pd(PPh₃)₄ (tetrakis(triphenylphosphine)-palladium(0)) and Pd₂(dba)₃. For relevant references see, for example, Ragan et al., Organic Process Research & Development 2003, 7(5), 675-683; and Gaare et al., Acta Chemica Scandinavica 1993, 47, 57-62.

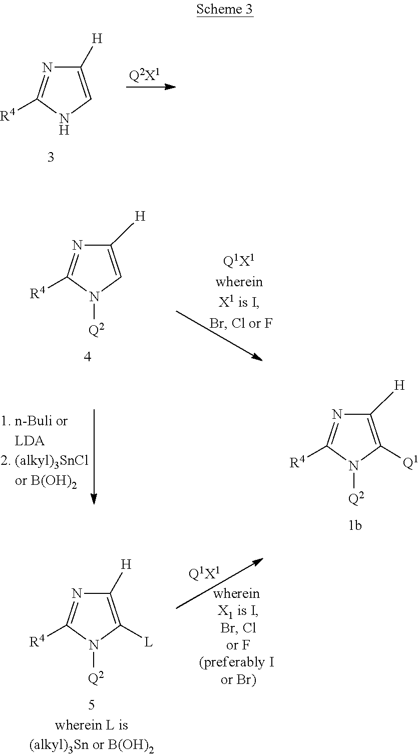

Compounds of Formula 1b can also be prepared as shown in Scheme 4. In this method a compound Formula 6 is first metallated with a metalating agent such as n-butyl lithium (n-BuLi), lithium diisopropylamide (LDA) or sodium hydride (NaH) in a solvent such as tetrahydrofuran, dioxane or toluene at temperatures ranging from about 0° C. to room temperature. The anion is then contacted with an electrophile resulting in the introduction of an R⁴ group onto Formula 6, thus providing a compound of Formula 1b. For halogenation, the electrophile can be a halogen derivative such as N-chlorosuccinimide (NCS), N-bromosuccinimide (NBS), N-iodosuccinimide (NIS), hexachloroethane or 1,2-dibromotetrachloroethane. For alkylation, the electrophile can be an alkylating agent of the formula R⁴-Lg (wherein Lg is a leaving group such as Cl, Br, I or a sulfonate, for example, p-toluenesulfonate, methanesulfonate or trifluoromethanesulfonate) where R⁴ is alkyl, alkylthio, haloalkyl, alkenyl, haloalkenyl, alkynyl, and the like. As referred to herein, the terms “alkylation” and “alkylating agent” are not limited to R⁴ being an alkyl group. For related reference see Almansa et al., Journal of Medicinal Chemistry 2003, 46, 3463-3475. Also, Example 4 illustrates the method of Scheme 4 using LDA and iodomethane.

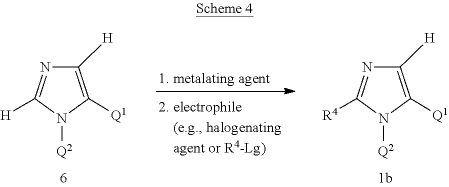

Compounds of Formula 6 are known and can be prepared by one of several methods disclosed herein. For example, using the method disclosed in Scheme 3, starting with a compound of Formula 3 wherein R⁴ is H provides compounds of Formula 6. Alternatively, compounds of Formula 6 can be prepared by the method of Scheme 6 described below using a compound of Formula 10 or 11 wherein R² is H (i.e. when R² is H, Formula 10 is p-toluenesulfonylmethyl isocyanide and Formula 11 is benzotriazol-1-yl-methyl isocyanide). For synthesis of a compound of Formula 6 using p-toluenesulfonylmethyl isocyanide see Step B of Examples 1 and 3.

In another method illustrated in Scheme 5, compounds of Formula 1b can be prepared by reacting N-chloroamidines of Formula 7 with enamines of Formula 8. In this method cyclization proceeds through the intermediacy of an in situ-generated 4-morpholino-4,5-dihydroimidazole which undergoes elimination of the morpholino group to provide the compounds of Formula 1b. Typically the reaction is run in the presence of a base such as pyridine, 4-(dimethylamino)pyridine or a trialkylamine and in a suitable solvent, such as dichloromethane, trichloromethane, carbon tetrachloride or toluene, at temperatures ranging from about 0° C. to the reflux temperature of the solvent. For a representative reference see Pocar et al., Tetrahedron Letters 1976, 21, 1839-1842. One skilled in the art will recognize that imidazole rings of Formula 1b can also be prepared by numerous other methods described in the chemistry literature. For example, the general method described by Wiglenda et al., Journal of Medicinal Chemistry 2007, 50(7), 1475-1484 can be used to prepare compounds of Formula 1b; the method can also be readily adapted to prepare Formula 1b compounds wherein each Q¹ and/or Q² is an optionally substituted benzyl group.

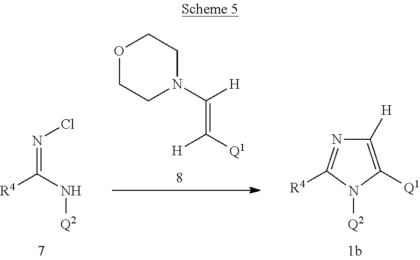

Compounds of Formula 7 can be easily synthesized from amidines and N-chlorosuccinimide according to the procedure given by Pocar et al., Tetrahedron Letters 1976, 21, 1839-1842. Enamines of Formula 8 can be prepared by known methods; for example, see van der Gen et al., Tetrahedron Letters 1979, 26, 2433-2436.

As shown in Scheme 6, compounds of Formula 1c can be prepared by reacting an imine of Formula 9 with a substituted p-toluenesulfonylmethyl isocyanide of Formula 10 or a substituted benzotriazol-1-ylmethyl isocyanide of Formula 11 in the presence of a suitable base such as potassium carbonate, potassium tert-butoxide, sodium hydroxide, sodium hydride, tert-butylamine or 1,8-diazabicyclo[5.4.0]undec-7-ene (DBU) in an appropriate solvent such as methanol, dioxane, tetrahydrofuran, dimethylsulfoxide, N,N-dimethylformamide or dimethoxyethane, at temperatures ranging from about 0 to 150° C. For reaction conditions see Almansa et al., Journal of Medicinal Chemistry 2003, 46(16), 3463-3475 and Katritzky et al., Heterocycles 1997, 44, 67-70. The method of Scheme 6 is also illustrated in Step B of Examples 9, 34 and 37.

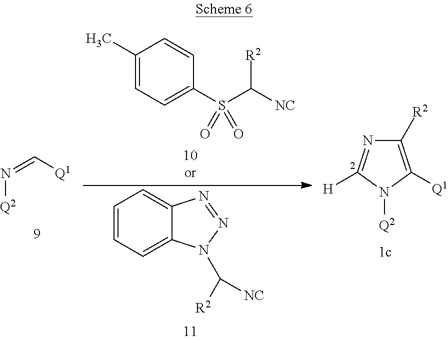

Imines of Formula 9 can be readily prepared by contacting an amine of Formula Q²NH₂ with an aldehyde of formula Q¹CHO under dehydrative conditions such as heating in toluene or xylenes with use of a Dean-Stark trap to remove water formed in the reaction. In some cases, an acid catalyst such as p-toluenesulfonic acid can be added to the reaction mixture to promote elimination of water. For representative procedures see Almansa et al., Journal of Medicinal Chemistry 2003, 46(16), 3463-3475. Also, Step A of Examples 1, 3, 9, 34 and 37 illustrate the preparation of a compound of Formula 9.

Compounds of Formula 10 can be prepared from the unsubstituted p-toluenesulfonylmethyl isocyanide under phase-transfer conditions using methods reported in the chemical literature; see, for example, Leusen et al., Tetrahedron Letters 1975, 40, 3487-3488.

The substituted benzotriazol-1-ylmethyl isocyanides of Formula 11 can be prepared by contacting benzotriazol-1-yl-methyl isocyanide with a compound of formula R²X¹ (wherein X¹ is halogen) in the presence of a base such as potassium carbonate, sodium hydride or potassium tert-butoxide. For typical reaction conditions see Katritzky et al., Heterocycles 1997, 44, 67-70. One skilled in the art will recognize other methods for preparing compounds of Formula 11, for example, the method described by Katritzky et al., Journal of the Chemical Society, Perkin Transactions 1, 1990, (7), 1847-1851.

Certain compounds of Formula 1c wherein R² is halogen can be prepared as shown in Scheme 7. In this method an aminonitrile of Formula 12 is combined with a methanaminium salt of Formula 13 to provide a compound of Formula 1c according to the general method taught by Pawar et al., Tetrahedron Letters 2006, 47, 5451-5453. The reaction is run in a suitable solvent such as dichloromethane or toluene at temperatures ranging from about 0° C. to the reflux temperature of the solvent. The method of Scheme 7 is illustrated in Step B of Examples 11 and 14.

Halogenation at the 2-position of the imidazole ring of Formula 1c can be achieved using methods analogous to those already described for Scheme 2 to provide compounds of Formula 1a wherein R² is halogen. Examples 12, 15, 30, 35 and 38 illustrate this halogenation method.

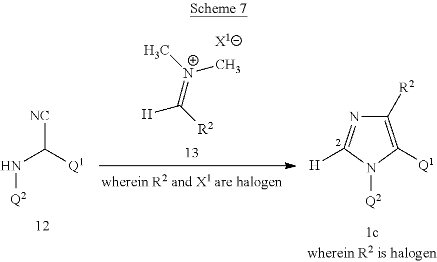

Aminonitriles of Formula 12 are readily available from amines of formula Q²NH₂, aldehydes of formula Q¹CHO and a cyanide source by means of the Strecker reaction. A variety of solvents and cyanide sources can be employed. The presence of a Lewis acid such as indium(III) chloride can be advantageous. For typical conditions, see, for example, Ranu et al., Tetrahedron, 2002, 58, 2529-2532. This reaction has been the subject of a number of reviews. For conditions and variations of this reaction see the following references and references cited therein: D. T. Mowry, Chemical Reviews 1948, 42, 236, H. Groeger, Chemical Reviews 2003, 103, 2795-2827, and M. North in Comprehensive Organic Functional Group Transformations A. R. Katritsky, O. Meth-Cohn and C. W. Rees Editors., Volume 3, 615-617; Pergamon, Oxford, 1995. Also the preparation of a compound of Formula 12 is illustrated in Step A of Examples 11 and 14. For less reactive amines of formula Q²NH₂, such as aryl amines containing ortho electron withdrawing groups, the use of trimethylsilylcyanide in combination with a catalyst such a guanidine hydrochloride can be advantageous. For a reference see, for example, Heydari et al., Journal of Molecular Catalysis A: Chemical 2007, 271(1-2), 142-144.

Methanaminium salts of Formula 13 are commercially available, for example, chloromethylenedimethyliminium chloride (i.e. R² and X¹ are Cl) can be obtained from commercial sources. Compounds of Formula 13 can also be synthesized by methods documented in the chemistry literature.

Other functionalized compounds of Formula 1a can be prepared as shown in Schemes 8 and 9. In Scheme 8, compounds of Formula 1c are used to provide the corresponding 2-imidazolecarboxaldehyde derivatives of Formula 1a¹ (i.e. Formula 1a wherein R⁴ is —CHO). In this reaction the imidazole ring is first lithiated at the 2-position using a lithium base such as lithium diisopropylamide (LDA), followed by treatment of the anion with N,N-dimethylformamide (DMF) provides the 2-imidazolecarboxaldehyde derivative. The method of Scheme 8 is illustrated in Example 20.

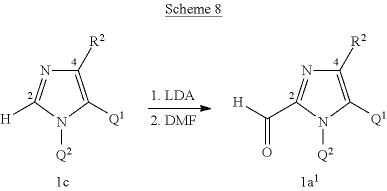

As shown in Scheme 9, the 2-imidazolecarboxaldehyde of Formula 1a¹ can be reduced with sodium borohydride in methanol to provide the corresponding compound of Formula 1a² (i.e. Formula 1a wherein R⁴ is 2-hydroxymethyl). For reaction conditions see Quan et al., Journal of Medicinal Chemistry 2005, 48(6), 1729-1744. Also for the preparation of a 2-hydroxymethyl derivative using the method of Scheme 9 see Example 21. Treatment of the 2-hydroxymethyl compound of Formula 1a² with diethylaminosulfur trifluoride (DAST) results in the 2-fluoromethyl derivative of Formula 1a³ (i.e. Formula 1a wherein R⁴ is —CH₂F). For reaction conditions see C. J. Wang, Organic Reactions 2005, Vol. 34 (Wiley, New York, 1951) Chapter 2, pp. 319-321. Also the method of Scheme 9 for the preparation of a 2-fluoromethyl derivative is illustrated in Example 22. Other 2-halomethyl analogs of Formula 1a³ can be prepared using methods described in the chemistry literature. For example, 2-bromomethyl analogs of Formula 1a³ can be prepared by treating 2-hydroxymethyl compounds of Formula 1a² with hydrobromic acid in a solvent such as glacial acetic acid using the method described by Beukers et al., Journal of Medicinal Chemistry 2004, 47(15), 3707-3709.

Also as shown in Scheme 9, the 2-imidazolecarboxaldehyde can be treated with hydroxylamine hydrochloride to provide the oxime of Formula 1a⁴ (i.e. Formula 1a wherein R⁴ is oxime functionality). For reaction conditions see Oresmaa et al., Journal of Medicinal Chemistry 2005, 48(13), 4231-4236, and Example 23. Alternatively, 2-imidazolecarboxaldehyde compounds of Formula 1^{a1} can be treated with a hydroxylamine salt of formula H₂NOR¹¹.HCl to provide compounds of Formula 1 wherein R⁴ is —CH═NOR¹¹. For a relevant reference, see PCT Patent Publication WO 2006/086634.

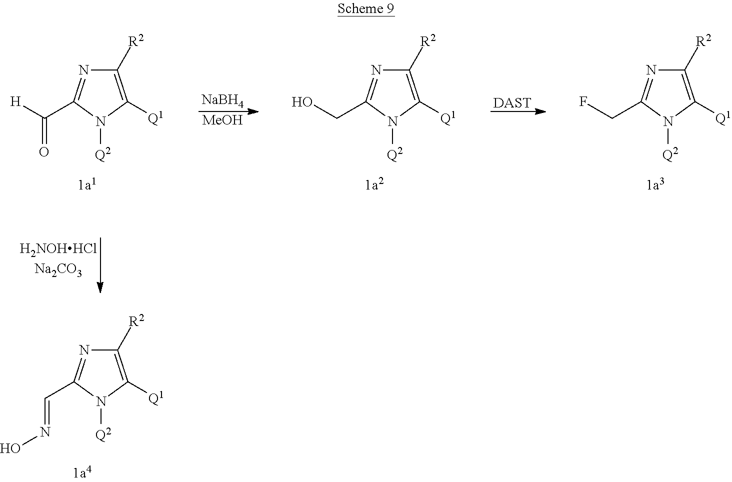

Schemes 5 through 7 are representative of just a few methods of preparing imidazole rings of the present invention. One skilled in the art will recognize that imidazole rings of Formula 1a can also be prepared by numerous other methods described in the chemistry literature. For leading references on imidazole synthesis see Grimmett in Imidazole and Benzimidazole Synthesis, Academic Press, California; and Grimmett, Science of Synthesis 2002, 12, 325-528.

Compounds of Formula 1d (i.e. Formula 1 wherein J is R¹, X is CQ³, Y is CR³ and Z is N) can be prepared as shown in Scheme 10 by cyclization of a 1,3-dicarbonyl compound of Formula 14 with an appropriately substituted hydrazine of formula NH₂NHR¹ in a suitable solvent such as ethanol, methanol, acetonitrile, glacial acetic acid, or mixtures thereof. The reaction is conducted at a temperature between about room temperature to the reflux temperature of the solvent and optionally in the presence of a base such as a metal carbonate, acetate or alkoxide. Two regioisomers can result from these types of reactions; however the desired regioisomer can be predominately formed by adjusting reaction conditions (e.g., solvent choice). For a general reference see, for example, Sliskovic et al., Journal of Medical Chemistry 1990, 33, 31-38 and Singh et al., Journal of Heterocyclic Chemistry 1989, 26, 733-738.

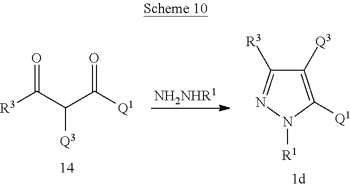

Alternatively, compounds of Formula 1d can be prepared as outlined in Scheme 11. In this method compounds of Formula 15 are first halogenated analogous to the method described in Scheme 2 to provide the compounds of Formula 16, which can then be coupled with a boronic acid of formula Q³B(OH)₂ using well-known Suzuki palladium-catalyzed cross coupling reaction conditions. Many catalysts are useful for the Suzuki reaction; particularly useful catalysts include tetrakis(triphenylphosphine)palladium(0) and [1,1′-bis(diphenylphosphino)ferrocene]dichloropalladium(II). Solvents such as tetrahydrofuran, acetonitrile, diethyl ether and dioxane are suitable. Many boronic acids of formula Q³B(OH)₂ are commercially available and others can be prepared by known methods. For a reference see, for example, Suzuki et al., Chemical Review 1995, 95, 2457-2483. Compounds of Formula 1d wherein Q³ is a N-linked heteroaromatic ring can be prepared via a palladium-catalyzed cross-coupling reaction using compounds of formula Q³H. For leading references see, for example, Buchwald et al., Accounts of Chemical Research, 1998, 31(12), 805-818 and Hartwig, Angew. Chem. Int. Ed., 1998, 37, 2046-2067. Also, Example 40, Steps B-C illustrate the method of Scheme 11.

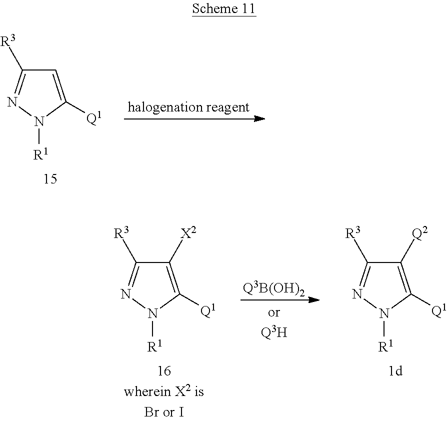

Compounds of Formula 15 can be prepared by the method of Scheme 10 using a hydrazine of formula NH₂NHR¹ and a dione of Formula 14 where Q³ is replaced by H. For a reference see Quan et al., Journal of Medicinal Chemistry 2005, 48(6), 1729-1744. For a specific example see Step A of Example 40.

In another method, as shown in Scheme 12, compounds of Formula 1d can be prepared by introduction of the R¹ substituent via alkylation of the pyrazole ring with an alkylating agent R¹-Lg wherein Lg is a leaving group such as Cl, Br, I or a sulfonate such as p-toluenesulfonate, methanesulfonate or trifluoromethanesulfonate. As referred to herein, the terms “alkylation” and “alkylating agent” are not limited to R¹ being an alkyl group and include in addition to alkyl such groups as alkylthio, haloalkyl, alkenyl, haloalkenyl, alkynyl, and the like. Alkylation of pyrazoles using potassium carbonate in N,N-dimethylformamide or acetone are described by Kitazaki et al., Chem. Pharm. Bull. 2000, 48(12), 1935-1946 and Jeon et al., Journal of Fluorine Chemistry 2007, 128, 1191-1197. One skilled in the art recognizes that a variety of bases and solvents can be used for alkylation of pyrazoles; for example, Alabaster et al., Journal of Med. Chemistry 1989, 32, 575-583 discloses use of sodium carbonate in N,N-dimethylformamide, Wang et al., Organic Letters 2000, 2(20), 3107-3109 discloses use of potassium tert-butoxide in methyl sulfoxide, and European Patent Application Publication EP-1081146-A1 describes the use of sodium or potassium hydroxide in methyl sulfoxide and in the presence of a phase transfer catalyst. For an example illustrating the method of Scheme 12 using sodium hydride in N,N-dimethylformamide see Step E of Example 39. One skilled in the art also recognizes that a variety of alternative synthetic methods are applicable for preparing compounds of Formula 1d including, for example, condensation with aryl iodides in the presence of copper(I) iodide and trans-cyclohexanediamine as reported by Buchwald et al., J. Am. Chem. Soc. 2001, 123, 7727-7729, and condensation with aryl boronic acids in the presence of copper(II) acetate and pyridine as reported by Lam et al., Tetrahedron Letters 1998, 39, 2941-2944. In some cases, depending on the reaction conditions, two regioisomers can be formed; the regioisomers can be separated by methods known to those skilled in the art, including chromatography.

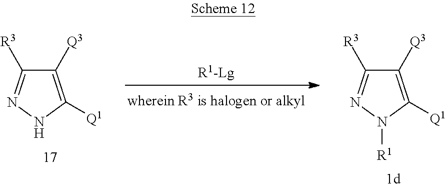

Starting compounds of Formula 17 wherein R³ is halogen or alkyl can be prepared from the corresponding compounds wherein R³ is H by halogenation or alkylation analogous to the method described in Scheme 1. For reaction conditions see, in addition to the references cited in Scheme 1, U.S. patent application publication 2007/054896 and Toto et al., Synthetic Communications 2008, 38(5), 674-683. For a specific example, see Step D of Example 39. Preparation of compounds of Formula 17 where in R³ is H (i.e. the precursor to Formula 17) can be accomplished by condensing compounds of Formula 18 with hydrazine as shown in Scheme 13. The reaction can be run in a variety of solvents, but typically optimal yields are obtained when the reaction is run in ethanol at a temperature between about room temperature and the reflux temperature of the solvent. General procedures for this type of reaction are well documented in the chemical literature; for example, see Maya et al., Bioorganic & Medicinal Chemistry 2005, 13(6), 297-2107; and Domagala et al., Journal of Heterocyclic Chemistry 1989, 26, 1147-1158. The method of Scheme 13 is also illustrated in Step C of Example 39.

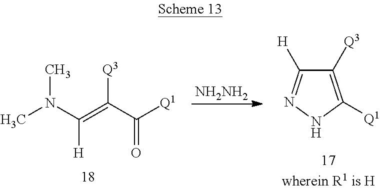

Alternatively, alkylhydrazines (i.e. R¹NHNH₂) can be used in place of hydrazine in the method of Scheme 13 to provide compounds of Formula 17 wherein R¹ is other than hydrogen (e.g., alkyl). Typically these reactions result in mixtures of 1- and 2-substituted pyrazole regioisomers which can be separated using chromatography.

Compounds of Formula 18 can be prepared from ketones of Formula 19 and N,N-dimethylformamide dimethyl acetal using the method described by Maya et al., Bioorganic & Medicinal Chemistry 2005, 13(6), 297-2107. The reaction is typically conducted in a solvent such as benzene, toluene or xylenes at a temperature between about room temperature and the reflux temperature of the solvent. The method of Scheme 14 is illustrated in Step B of Example 39.

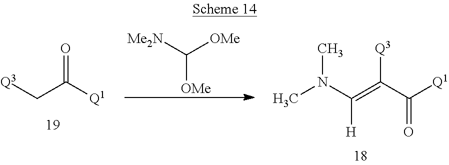

Ketones of Formula 19 can be prepared by reaction of acid chlorides of Formula 20 with the desired aromatic species of formula Q¹-H under Friedel-Crafts condensation reaction conditions. Friedel-Crafts reactions are documented in a variety of published references including Lutjens et al., Journal of Medicinal Chemistry 2003, 46(10), 1870-1877; PCT Patent Publication WO 2005/037758; and J. March, Advanced Organic Chemistry, McGraw-Hill, New York, p 490 and references cited within. The method of Scheme 15 is also illustrated in Step A of Example 39.

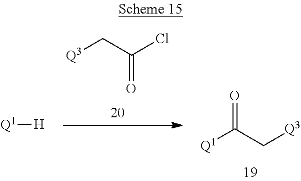

In another method, compounds of Formula 1d wherein R³ is halogen or cyano can be synthesized as shown in Scheme 16. In the first step, cyclization of 2-cyanoketones of Formula 23 with hydrazines of formula NH₂NHR¹ in a suitable solvent such as ethanol, methanol or glacial acetic acid gives 3-aminopyrazoles of Formula 24. Cyclization reactions of this type are documented in the chemical literature; see, for example, Compton et al., Journal of Medical Chemistry 2004, 47, 5872-5893. Typically these reactions result in mixtures of 1- and 2-substituted pyrazole regioisomers which can be separated using chromatography. Using Sandmeyer reaction conditions, amines of Formula 24 can be converted to diazonium salts and then reacted with a copper salt (e.g., copper(I) halide, copper(II) halide or copper(I) cyanide) in the presence of an acid to provide compounds of Formula 1d. The diazonium salt formed from the amine of Formula 24 is generated under standard conditions, for example, strong acid (e.g., hydrochloric acid or hydrobromic acid) and sodium nitrite or using non-aqueous conditions. For a review of the Sandmeyer reaction see Hodgson, Chemical Reviews 1947, 40(2), 251-277. Also, copper chloride, tent-butyl nitrite and acetonitrile can be used according to the general method disclosed by South, Journal of Heterocyclic Chemistry 1991, 28, 1003-1011.

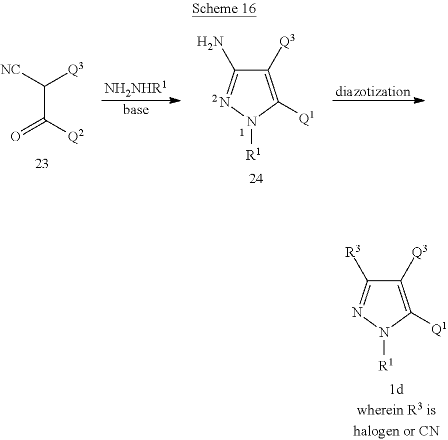

Compounds of Formula 1e (i.e. Formula 1 wherein J is Q², X is CR², Y is N and Z is N) can be prepared by cycloaddition of alkynes to azides as illustrated in Scheme 17. In this method bromomagnesium or lithium acetylides are first generated by reaction of an alkyne of Formula 25 with a Grignard reagent or an alkyl lithium base, followed by reaction with an azide of Formula 26. The cyclization reaction proceeds through the intermediacy of an in situ-generated 4-metallotriazole which when treated with an electrophile of formula R²-Lg (wherein Lg is a leaving group such as Cl, Br, I or a sulfonate, for example, p-toluenesulfonate, methanesulfonate or trifluoromethanesulfonate) provides the corresponding compound of Formula 1e. Typically the reaction is run in an aprotic solvent, such as tetrahydrofuran, at temperature between about 0° C. to the reflux temperature of the solvent. For a representative reference see Krasinski et al., Organic Letters 2004, 6(8), 1237-1240. The method of Scheme 17 is illustrated in Step B of Example 42.

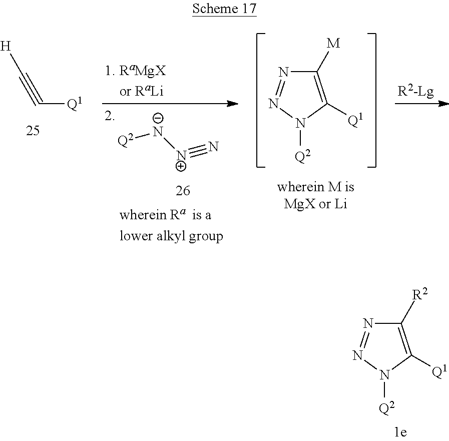

Compounds of Formula 1 and the intermediates described above can be subjected to various electrophilic, nucleophilic, radical, organometallic, oxidation and reduction reactions to add or modify substituents for formation of further compounds of Formula 1. Compounds wherein R², R³, R⁴, R^{5a}, R^{5b} or R^{5c} is halogen (preferably bromide or iodide) are particularly useful intermediates for transition metal-catalyzed cross-coupling reactions to prepare compounds of Formula 1. These types of reactions are well documented in the literature; see, for example, Tsuji in Transition Metal Reagents and Catalysts: Innovations in Organic Synthesis, John Wiley and Sons, Chichester, 2002; Tsuji in Palladium in Organic Synthesis, Springer, 2005; and Miyaura and Buchwald in Cross-Coupling Reactions: A Practical Guide, Springer, 2002; and references cited therein.

One skilled in the art will recognize that for some compounds of Formula 1, the R^{5a}, R^{5b} and R^{5c} substituents attached to the rings and ring systems of Q¹, Q² and Q³ may be more conveniently incorporated after forming the central azole ring with the rings or ring systems of Q¹, Q² and Q³ attached. In particular, when R^{5a}, R^{5b} and/or R^{5c} is halogen or another suitable leaving group, the leaving group can be replaced using various electrophilic, nucleophilic and organometallic reactions known in the art to introduce other functional groups as R^{5a}, R^{5b} and R^{5c}. Example 29 demonstrates the preparation of a compound of Formula 1a wherein R^{5a} is cyano (—CN) starting from the corresponding compound of Formula 1a wherein R^{5a} is bromo. Example 43 illustrates the preparation of a compound of Formula 1a wherein R^{5b} is thiocyanate (—SCN) starting from the corresponding compound of Formula 1a wherein R^{5b} is iodo.

Furthermore, compounds of Formula 1 wherein a ring or ring system of Q¹, Q² or Q³ is substituted with an R^{5a}, R^{5b} or R^{5c} substituent which is -U-V-T (as defined in the Summary of the Invention) can be prepared from the corresponding compounds of Formula 1 wherein R^{5a}, R^{5b} or R^{5c} is a halogen or other suitable leaving group, such as by the general method described in PCT Patent Publication WO 2007/149448 (see Scheme 15 therein). This reference also describes other general methods for forming an R^{5a}, R^{5b} or R^{5c} substituent as -U-V-T (see Schemes 16-19 therein). Present Examples 32 through 33 demonstrate the preparation of a compound of Formula 1a wherein R^{5a} is -U-V-T (i.e. —O(CH₂)₃NHCH₃) starting from the corresponding compound of Formula 1a wherein R^{5a} is methoxy.

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2^{nd} ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula 1. One skilled in the art will also recognize that it may be necessary to perform a combination of the steps illustrated in the above schemes in an order other than that implied by the particular sequence presented to prepare the compounds of Formula 1.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. In the following Examples HPLC analyses were obtained on an Alltech Altima C18 analytical column with UV detection. The solvent system was solvent A: water with 0.05% trifluoroacetic acid by volume/volume, and solvent B: acetonitrile with 0.05% trifluoroacetic acid by volume/volume (gradient started at 0 minutes with 90% solvent A and 10% solvent B and increased solvent B to 90% over 20 minutes, flow rate was 1 mL/minute). Percentages are by weight except for chromatographic solvent mixtures or where otherwise indicated. Parts and percentages for chromatographic solvent mixtures are by volume unless otherwise indicated. The mass spectra value given in the following Examples is the molecular weight of the highest isotopic abundance parent ion (M+1) formed by addition of H⁺ (molecular weight of 1) to the molecule, observed by mass spectrometry using electrospray ionization (ESI). ¹H NMR spectra are reported in ppm downfield from tetramethylsilane; “s” means singlet, “d” means doublet, “m” means multiplet.

### Example 1

### Preparation of 4-chloro-1-(4-chlorophenyl)-5-(2,4,6-trifluorophenyl)-1H-imidazole (Compound 1)

### Step A: Preparation of (E)-4-chloro-N-[(2,4,6-trifluorophenyl)methylene]benzene

To a mixture of 2,4,6-trifluorobenzaldehyde (3.0 g, 18.7 mmol) in toluene (100 mL) was added 4-chloroaniline (2.39 g, 18.7 mmol). The reaction mixture was heated at reflux with the use of a Dean-Stark trap for the azeotropic removal of water. After 16 h the reaction mixture was cooled to room temperature and concentrated under reduced pressure. The resulting residue was purified by silica gel chromatography using ethyl acetate-hexanes (1:9) as eluant to provide the title compound as a pale-yellow solid (4.20 g).

¹H NMR (CDCl₃): δ 8.55 (s, 1H), 7.37-7.34 (m, 2H), 7.17-7.13 (m, 2H), 6.83-6.77 (m, 2H). ESI MS m/z 270 (M+1).

### Step B Preparation of 1-(4-chlorophenyl)-5-(2,4,6-trifluorophenyl)-1H-imidazole

To a mixture of (E)-4-chloro-N-[(2,4,6-trifluorophenyl)methylene]benzene (i.e. the product of Step A) (4.20 g, 15.6 mmol) in methanol and 1,2-dimethoxyethane (2:1, 152 mL) was added p-toluenesulfonylmethyl isocyanide (4.57 g, 23.4 mmol) and potassium carbonate (4.30 g, 31.2 mmol). The reaction mixture was heated at 85° C. for 4 h, cooled, and then concentrated under reduced pressure. The resulting residue was diluted with ethyl acetate (200 mL), washed with water (75 mL) and saturated aqueous sodium chloride (75 mL), dried over sodium sulfate, filtered and concentrated under reduced pressure. The resulting material was purified by silica gel chromatography using ethyl acetate-hexanes (1:4) as eluant to provide the title compound as a pale yellow solid (1.30 g, 98.9 area % purity by HPLC) melting at 170-172° C.

¹H NMR (DMSO-d₆): δ 7.29 (s, 1H), 7.11-7.07 (m, 2H), 6.68-6.63 (m, 2H).

MS m/z 309 (M+1).

### Step C Preparation of 4-chloro-1-(4-chlorophenyl)-5-(2,4,6-trifluorophenyl)-1H-imidazole

To a mixture of 1-(4-chlorophenyl)-5-(2,4,6-trifluorophenyl)-1H-imidazole (i.e. the product of Step B) (0.100 g, 0.32 mmol) in chloroform (2 mL) was added N-chlorosuccinimide (0.046 g, 0.34 mmol). The reaction mixture was heated at reflux for 16 h and then cooled to room temperature. The reaction mixture was diluted with chloroform (100 mL), washed with water (55 mL) and saturated aqueous sodium chloride (55 mL), dried over sodium sulfate, filtered and concentrated under reduced pressure. The resulting material was purified by silica gel chromatography using ethyl acetate-hexanes (1:4) as eluant to provide the title compound, a compound of the present invention, as an off-white solid (0.075 g, 98.9 area % purity by HPLC) melting at 102-104° C.

¹H NMR (CDCl₃): δ 7.68 (s, 1H), 7.37-7.33 (m, 2H), 7.11-7.07 (m, 2H), 6.74-6.65 (m, 2H).

ESI MS m/z 343 (M+1).

### Example 2

### Preparation of 2,4-dichloro-1-(4-chlorophenyl)-5-(2,4,6-trifluorophenyl)-1H-imidazole (Compound 6)

To a mixture of 1-(4-chlorophenyl)-5-(2,4,6-trifluorophenyl)-1H-imidazole (i.e. the product of Step B of Example 1) (0.280 g, 0.90 mmol) in chloroform (10 mL) was added N-chlorosuccinimide (0.420 g, 3.14 mmol). The reaction mixture was heated at reflux for 16 h and then cooled to room temperature. The reaction mixture was diluted with chloroform (100 mL), washed with water (55 mL) and saturated aqueous sodium chloride solution (55 mL), dried over sodium sulfate, filtered and concentrated under reduced pressure. The resulting material was purified by silica gel chromatography using ethyl acetate-hexanes (1:4) as eluant to provide the title compound, a compound of the present invention, as a pale-yellow solid (0.23 g, 96.7 area % purity by HPLC) melting at 112-119° C.

¹H NMR (CDCl₃): δ 7.40-7.35 (m, 2H), 7.14-7.10 (m, 2H), 6.70-6.61 (m, 2H).

ESI MS m/z 377 (M+1).

### Example 3

### Preparation of 2,4-dichloro-1-(4-chlorophenyl)-5-(2,6-difluorophenyl)-1H-imidazole (Compound 3)

### Step A: Preparation of (E)-4-chloro-N-[(2,6-difluorophenyl)methylene]benzene

To a mixture of 2,6-difluorobenzaldehyde (4.0 g, 18.7 mmol) in toluene (100 mL) was added 4-chloroaniline (3.60 g, 28.0 mmol). The reaction mixture was heated at reflux with the use of a Dean-Stark trap for azeotropic removal of water. After 16 h the reaction mixture was cooled to room temperature and concentrated under reduced pressure. The resulting residue was purified by silica gel chromatography using ethyl acetate-hexanes (0.5:9.5) as eluant to provide the title compound as a pale-yellow solid (6.20 g).

¹H NMR (CDCl₃): δ 8.64 (s, 1H), 7.44-7.33 (m, 3H), 7.19-7.14 (m, 2H), 7.04-6.96 (m, 2H).

### Step B Preparation of 1-(4-chlorophenyl)-5-(2,6-difluorophenyl)-1H-imidazole

To a mixture of (E)-4-chloro-N-[(2,6-difluorophenyl)methylene]benzene (i.e. the product of Step A) (4.0 g, 16.0 mmol) in methanol and 1,2-dimethoxyethane (7:3, 160 mL) was added p-toluenesulfonylmethyl isocyanide (4.67 g, 24.0 mmol) and potassium carbonate (4.65 g, 24.0 mmol). The reaction mixture was heated at 85° C. for 4 h, cooled, and then concentrated under reduced pressure. The resulting residue was diluted with ethyl acetate (200 mL), washed with water (75 mL) and saturated aqueous sodium chloride solution (75 mL), dried over sodium sulfate, filtered and concentrated under reduced pressure. The resulting material was purified by silica gel chromatography using ethyl acetate-hexanes (3:7) as eluant to provide the title compound as a pale-yellow solid (1.40 g, 98.9 area % purity by HPLC) melting at 170-172° C.

¹H NMR (CDCl₃): δ 7.79 (d, J=0.9 Hz, 1H), 7.34-7.29 (m, 4H), 7.12-7.08 (m, 2H), 6.91-6.83 (m, 2H).

ESI MS m/z 291 (M+1).

### Step C Preparation of 2,4-dichloro-1-(4-chlorophenyl)-5-(2,6-difluorophenyl)-1H-imidazole

To a mixture of 1-(4-chlorophenyl)-5-(2,6-trifluorophenyl)-1H-imidazole (i.e. the product of Step B) (0.60 g, 2.06 mmol) in chloroform (24 mL) was added N-chlorosuccinimide (0.69 g, 5.17 mmol). The reaction mixture was heated at reflux for 16 h and then cooled to room temperature. The reaction mixture was diluted with chloroform (40 mL), washed with water (30 mL) and saturated aqueous sodium chloride solution (30 mL), dried over sodium sulfate, filtered and concentrated under reduced pressure. The resulting residue was purified by silica gel chromatography using ethyl acetate-hexanes (1:4) as eluant to provide the title compound, a compound of the present invention, as an off-white solid (0.50 g, 97.9 area % purity by HPLC) melting at 123-125° C.

¹H NMR (CDCl₃): δ 7.40-7.30 (m, 3H), 7.16-7.10 (m, 2H), 6.90-6.84 (m, 2H).

ESI MS m/z 360 (M+1).

### Example 4

### Preparation of 1-(4-chlorophenyl)-5-(2,6-difluorophenyl)-2-methyl-1H-imidazole

To a stirred mixture of 1-(4-chlorophenyl)-5-(2,6-trifluorophenyl)-1H-imidazole (i.e. the product of Step B of Example 3) (1.00 g, 3.44 mmol) in tetrahydrofuran (34 mL) at −50° C. was added dropwise a solution of lithium diisopropylamide (1.0 M in tetrahydrofuran, 2.60 mL, 5.10 mmol). The reaction mixture was stirred at −50° C. for 1.5 h, and then a solution of iodomethane (1.47 g, 10.3 mmol) in tetrahydrofuran (16 mL) was added. The reaction mixture was slowly warmed to room temperature, stirred for 4 h, and then concentrated under reduced pressure. The resulting residue was diluted with dichloromethane (50 mL), washed with water (20 mL) and saturated aqueous sodium chloride solution (20 mL), dried over sodium sulfate, filtered and concentrated under reduced pressure. The resulting residue was purified by silica gel chromatography using ethyl acetate-hexanes (1:4) as eluant to provide the title compound, a compound of the present invention, as a pale-yellow solid (0.74 g).

¹H NMR (CDCl₃): δ 7.24-7.17 (m, 1H), 7.14 (s, 1H), 7.12-7.06 (m, 2H), 6.85-6.78 (m, 2H), 2.33 (s, 3H).

ESI MS m/z 305 (M+1).

### Example 5

### Preparation of 4-chloro-1-(4-chlorophenyl)-5-(2,6-difluorophenyl)-2-methyl-1H-imidazole (Compound 2)

To a stirred mixture of 1-(4-chlorophenyl)-5-(2,6-difluorophenyl)-2-methyl-1H-imidazole (i.e. the product of Example 4) (0.740 g, 2.40 mmol) in chloroform (22 mL) was added N-chlorosuccinimide (0.34 g, 2.55 mmol). The reaction mixture was heated at reflux for 16 h and then cooled to room temperature. The reaction mixture was diluted with chloroform (50 mL), washed with water (30 mL), saturated aqueous sodium chloride solution (30 mL) and dried over sodium sulfate, filtered and concentrated under reduced pressure. The resulting residue was purified by silica gel chromatography using ethyl acetate-hexanes (1:9) as eluant to provide the title compound, a compound of the present invention, as an off-white solid (0.35 g, 98.0 area % purity by HPLC) melting at 143-145° C.

¹H NMR (CDCl₃): δ 7.35-7.29 (m, 3H), 7.10-7.06 (m, 2H), 6.90-6.83 (m, 2H), 2.33 (s, 3H).

ESI MS m/z 339 (M+1).

### Example 6

### Preparation of 2-bromo-1-(4-chlorophenyl)-5-(2,6-difluorophenyl)-2-methyl-1H-imidazole (Compound 740)

To a mixture of 1-(4-chlorophenyl)-5-(2,6-difluorophenyl)-2-methyl-1H-imidazole (prepared by the method of Example 4) (0.300 g, 0.97 mmol). The reaction mixture was heated at reflux for 16 h, and then cooled to room temperature. The reaction mixture was diluted with chloroform (20 mL), washed with water (5 mL) and saturated aqueous sodium chloride solution (5 mL), dried over sodium sulfate, filtered and concentrated under reduced pressure. The resulting residue was purified by silica gel chromatography using ethyl acetate-hexanes as eluant to provide the title compound, a compound of the present invention, as a light yellow solid (269 mg) melting at 183-185° C.

¹H NMR (CDCl₃): δ 7.35-7.31 (m, 3H), 7.09-7.06 (d, J=8.7 Hz, 2H), 6.88-6.82 (t, J=7.8 Hz, 2H), 2.31 (s, 3H).

ESI MS m/z 383 (M+1).

### Example 7

### Preparation of 1-(4-chlorophenyl)-5-(4-fluorophenyl)-2-methyl-1H-imidazole (Compound 4)

### Step A: Preparation of 1-(4-chlorophenyl)-2-methyl-1H-imidazole

To a mixture of 1-chloro-4-iodobenzene (1.50 g, 6.30 mmol) in N,N-dimethylformamide (10 mL) was added cesium carbonate (3.50 g, 10.9 mmol), copper(I) iodide (0.10 g, 0.50 mmol), 2-methylimidazole (0.46 g, 5.66 mmol) and. (1R,2R)-1,2-cyclohexanediamine (0.12 g, 1.00 mmol). The reaction mixture was heated at 120° C. for 16 h and then cooled to room temperature. The reaction mixture was diluted with ethyl acetate (80 mL), washed with water (2×30 mL) and saturated aqueous sodium chloride solution (40 mL), dried over sodium sulfate, filtered and concentrated under reduced pressure. The resulting residue was purified by silica gel chromatography using ethyl acetate-hexanes (0.5:9.5) as eluant to provide the title compound as a brown solid (0.60 g).

¹H NMR (CDCl₃): δ 7.48-7.44 (m, 2H), 7.26-7.22 (m, 2H), 7.05 (d, J=16.8 Hz, 2H), 2.35 (s, 3H).

ESI MS m/z 193 (M+1).

### Step B Preparation of 1-(4-chlorophenyl)-5-(4-fluorophenyl)-2-methyl-1H-imidazole

To a mixture of 1-(4-chlorophenyl)-2-methyl-1H-imidazole (i.e. the product of Step A) (0.700 g, 0.520 mmol) in N,N-dimethylformamide (10 mL) was added 1-fluoro-4-iodobenzene (1.61 g, 7.30 mmol), tris(2-methylphenyl)phosphine (0.110 g, 0.360 mmol), cesium fluoride (1.10 g, 7.30 mmol) and palladium(II) acetate (0.041 g, 0.18 mmol). The reaction mixture was stirred under argon, heated at reflux for 16 h, and then cooled to room temperature. The reaction mixture was diluted with ethyl acetate (40 mL), washed with water (20 mL) and saturated aqueous sodium chloride solution (20 mL), dried over sodium sulfate, filtered and concentrated under reduced pressure. The resulting material was purified by silica gel chromatography using methanol-dichloromethane (1:9) as eluant to provide the title compound, a compound of the present invention, as an off-white solid (0.20 g, 95.3 area % purity by HPLC) melting at 132-134° C.

¹H NMR (CDCl₃): δ 7.42-7.39 (m, 2H), 7.11-7.06 (m, 3H), 7.05-6.99 (m, 2H), 6.95-6.87 (m, 2H), 2.31 (s, 3H).

ESI MS m/z 287 (M+1).

### Example 8

### Preparation of 4-chloro-1-(4-chlorophenyl)-5-(4-fluorophenyl)-2-methyl-1H-imidazole (Compound 5)

To a mixture of 1-(4-chlorophenyl)-5-(4-fluorophenyl)-2-methyl-1H-imidazole (i.e. the product of Step B of Example 7) (0.100 g, 0.340 mmol) in chloroform (2.5 mL) was added N-chlorosuccinimide (0.05 g, 0.36 mmol). The reaction mixture was heated at reflux for 16 h and then cooled to room temperature. The reaction mixture was diluted with chloroform (20 mL), washed with water (10 mL) and saturated aqueous sodium chloride solution (10 mL), dried over sodium sulfate, filtered and concentrated under reduced pressure. The resulting residue was purified by silica gel chromatography using ethyl acetate-hexanes (1:4) as eluant to provide the title compound, a compound of the present invention, as a yellow solid (0.065 g, 95.2 area % purity by HPLC) melting at 124-126° C.

¹H NMR (CDCl₃): δ 7.40-7.36 (m, 2H), 7.14-7.07 (m, 2H), 7.06-7.03 (m, 2H), 6.99-6.92 (m, 2H), 2.29 (s, 3H).

ESI MS m/z 322 (M+1).

### Example 9

### Preparation of 5-(2,6-difluoro-4-methoxyphenyl)-1-(3,5-dimethoxyphenyl)-4-methyl-1H-imidazole (Compound 632)

### Step A: Preparation of N-[(2,6-difluoro-4-methoxyphenyl)methylene]-3,5-dimethoxybenzenamine

A mixture of 3,5-dimethoxybenzamine (2.00 g, 13.1 mmol) and 2,6-difluoro-4-methoxybenzaldehyde (2.30 g, 13.1 mmol) in toluene (40 mL) was heated at reflux overnight with use of a Dean-Stark trap for azeotropic removal of water. The reaction mixture was cooled to room temperature and concentrated under reduced pressure to provide the title compound as a solid (4.00 g).

¹H NMR (CDCl₃): δ 8.56 (s, 1H), 6.53 (m, 2H), 6.36 (m, 3H), 3.85 (s, 3H), 3.81 (s, 6H).

### Step B Preparation of 5-(2,6-difluoro-4-methoxyphenyl)-1-(3,5-dimethoxyphenyl)-4-methyl-1H-imidazole

A mixture of N-[(2,6-difluoro-4-methoxyphenyl)methylene]-3,5-dimethoxy-benzenamine (i.e. the product of Step A) (1.80 g, 6.0 mmol), 1-[(1-isocyanoethyl)sulfonyl]-4-methylbenzene (1.90 g, 9.0 mmol) and potassium tert-butoxide in tetrahydrofuran (20 mL) was heated at reflux overnight. The reaction mixture was cooled and then concentrated under reduced pressure. The resulting material was purified by medium pressure liquid chromatography on silica gel (0 to 100% gradient of ethyl acetate in hexanes as eluant) to provide the title compound, a compound of the present invention, as a solid (0.20 g).

¹H NMR (CDCl₃): δ 7.73 (s, 1H), 6.45 (m, 2H), 6.38 (s, 1H), 6.27 (s, 2H), 3.78 (s, 3H), 3.70 (m, 6H), 2.18 (s, 3H).

### Example 10

### Preparation of 2-chloro-5-(2,6-difluoro-4-methoxyphenyl)-1-(3,5-dimethoxyphenyl)-4-methyl-1H-imidazole (Compound 694)

To a mixture of 5-(2,6-difluoro-4-methoxyphenyl)-1-(3,5-dimethoxyphenyl)-4-methyl-1H-imidazole (i.e. the product of Example 9) (0.280 g, 0.78 mmol) and hexachloroethane (1.10 g, 4.7 mmol) in tetrahydrofuran (5 mL) at −78° C. was added lithium diisopropylamide (1.0 M in tetrahydrofuran, 0.390 mL, 0.78 mmol). After 1 h more lithium diisopropylamide (1.0 M in tetrahydrofuran, 0.150 mL, 0.30 mmol) was added to the reaction mixture, stirring was continued for an additional 1 h, and then more lithium diisopropylamide (1.0 M in tetrahydrofuran, 0.150 mL, 0.30 mmol) was added to the reaction mixture. The reaction mixture was allowed to slowly warm to room temperature, stirred for 2.5 h, and then concentrated under reduced pressure. The resulting material was purified by medium pressure liquid chromatography on silica gel (0 to 100% gradient of ethyl acetate in hexanes as eluant) to provide the title compound, a compound of the present invention, as a solid (0.011 g).

¹H NMR (CDCl₃): δ 6.42 (m, 3H), 6.31 (m, 2H), 3.77 (s, 3H), 3.72 (s, 6H), 2.13 (s, 1H).

### Example 11

### Preparation of 4-chloro-5-(2,6-difluoro-4-methoxyphenyl)-1-(3-fluorophenyl)-1H-imidazole (Compound 246)

### Step A: Preparation of 2,6-α-[(3-fluorophenyl)amino)]-4-methoxybenzeneacetonitrile

A mixture of 3-fluoroaniline (1.15 g, 10.4 mmol), 2,6-difluoro-4-methoxybenzaldehyde (2.00 g, 11.6 mmol), potassium cyanide (2.70 g, 41.6 mmol) and indium chloride (2.30 g, 10.4 mmol) in tetrahydrofuran (40 mL) was stirred at room temperature overnight. The reaction mixture was diluted with water (about 100 mL) and extracted with ethyl acetate. The combined ethyl acetate extracts were concentrated under reduced pressure to provide the title compound as an oil, which was carried directly on to Step B.

### Step B Preparation of 4-chloro-5-(2,6-difluoro-4-methoxyphenyl)-1-(3-fluorophenyl)-1H-imidazole

To a mixture of 2,6-α-[(3-fluorophenyl)amino)]-4-methoxybenzeneacetonitrile (i.e. the product of Step A) (10.4 mmol) in dichloromethane (20 mL) was added N-(chloromethylene)-N-methylmethanaminium chloride (1.60 g, 12.5 mmol). The reaction mixture was heated to reflux for 3 h and then diluted with saturated aqueous sodium carbonate solution. The aqueous mixture was extracted with dichloromethane. The combined organic layers were dried over magnesium sulfate, filtered and concentrated under reduced pressure. The resulting material was purified by medium pressure liquid chromatography on silica gel (0 to 100% gradient of ethyl acetate in hexanes as eluant) to provide the title compound, a compound of the present invention, as a solid (2.27 g).

¹H NMR (CDCl₃): δ 7.37 (m, 1H), 7.13 (m, 1H), 6.96 (m, 2H), 6.41 (d, 2H), 3.77 (s, 3H).

### Example 12

### Preparation of 2-bromo-4-chloro-5-(2,6-difluoro-4-methoxyphenyl)-1-(3-fluorophenyl)-1H-imidazole (Compound 256)

A stirred mixture of 4-chloro-5-(2,6-difluoro-4-methoxyphenyl)-1-(3-fluorophenyl)-1H-imidazole (i.e. the product of Step B of Example 11) (1.00 g, 3.0 mmol) and N-bromosuccinimide (0.641 g, 3.6 mmol) in N,N-dimethylformamide (15 mL) was heated at 60° C. for 2.5 days. The reaction mixture was diluted with saturated aqueous sodium carbonate solution, and the resulting aqueous mixture was extracted with dichloromethane. The combined organic layers were dried over magnesium sulfate, filtered and concentrated under reduced pressure. The resulting material was purified by medium pressure liquid chromatography on silica gel (0 to 100% gradient of ethyl acetate in hexanes as eluant) to provide the title compound, a compound of the present invention, as a solid (0.67 g).

¹H NMR (CDCl₃): δ 7.37 (m, 1H), 7.13 (m, 1H), 7.13 (m, 1H), 6.96 (m, 2H), 6.41 (d, 2H), 3.77 (s, 3H).

### Example 13

### Preparation of 4-chloro-5-(2,6-difluoro-4-methoxyphenyl)-1-(3-fluorophenyl)-2-methyl-1H-imidazole (Compound 280)

A mixture of 2-bromo-4-chloro-5-(2,6-difluoro-4-methoxyphenyl)-1-(3-fluorophenyl)-1H-imidazole (i.e. the product of Example 12) (0.200 g, 0.490 mmol), trimethylboroxine (0.062 g, 0.490 mmol), cesium carbonate (0.479 g, 1.47 mmol) and dichlorobis(triphenylphosphine)palladium (0.035 g, 0.05 mmol) in dioxane (5 mL) and water (2 drops) was heated at reflux overnight. More trimethylboroxine (0.062 g, 0.490 mmol) and dichlorobis(triphenylphosphine)palladium (0.035 g, 0.05 mmol) were added to the reaction mixture, and the mixture was again heated at reflux overnight. The reaction mixture was concentrated under reduced pressure, and the resulting material was purified by medium pressure liquid chromatography on silica gel (0 to 100% gradient of ethyl acetate in hexanes as eluant) to provide the title compound, a compound of the present invention, as a solid (0.090 g).

¹H NMR (CDCl₃): δ 7.35 (m, 1H), 7.09 (m, 1H), 6.94 (d, 1H), 6.89 (m, 1H), 6.39 (m, 2H), 3.76 (s, 3H), 2.31 (s, 3H).

### Example 14

### Preparation of 4-bromo-5-(2,6-difluoro-3-methoxyphenyl)-1-(4-fluorophenyl)-1H-imidazole (Compound 450)

### Step A: Preparation of 2,6-α-[(4-fluorophenyl)amino)]-3-methoxybenzeneacetonitrile

A mixture of 4-fluoroaniline (1.17 g, 10.6 mmol), 2,6-difluoro-3-methoxybenzaldehyde (2.00 g, 11.6 mmol), potassium cyanide (2.80 g, 42.4 mmol) and indium chloride (2.30 g, 10.4 mmol) in tetrahydrofuran (50 mL) was stirred at room temperature overnight. The reaction mixture was diluted with water (about 100 mL) and extracted with ethyl acetate. The combined ethyl acetate layers were concentrated under reduced pressure to provide the title compound as an oil, which was carried directly on to Step B.

### Step B Preparation of 4-bromo-5-(2,6-difluoro-3-methoxyphenyl)-1-(4-fluorophenyl)-1H-imidazole

To a mixture of 2,6-α-[(4-fluorophenyl)amino)]-3-methoxybenzeneacetonitrile (i.e. the product of Step A) (10.6 mmol) in dichloromethane (20 mL) was added N-(bromomethylene)-N-methylmethanaminium bromide (2.80 g, 12.7 mmol). The reaction mixture was heated to 80° C. for one minute, then saturated aqueous sodium carbonate solution was added and the aqueous mixture was extracted with dichloromethane. The combined organic layers were dried over magnesium sulfate, filtered and concentrated under reduced pressure. The resulting material was purified by medium pressure liquid chromatography on silica gel (0 to 100% gradient of ethyl acetate in hexanes as eluant) to provide the title compound, a compound of the present invention, as a solid (1.78 g).

¹H NMR (CDCl₃): δ 7.71 (m, 1H), 7.15 (m, 2H), 7.05 (m, 2H), 6.96 (m, 1H), 6.82 (m, 1H), 3.85 (s, 3H).

### Example 15

### Preparation of 2,4-dibromo-5-(2,6-difluoro-3-methoxyphenyl)-1-(4-fluorophenyl)-1H-imidazole (Compound 504)

A stirred mixture of 4-bromo-5-(2,6-difluoro-3-methoxyphenyl)-1-(4-fluorophenyl)1H-imidazole (i.e. the product of Step B of Example 14) (0.500 g, 1.3 mmol) and N-bromosuccinimide (0.285 g, 1.60 mmol) in N,N-dimethylformamide (15 mL) was heated at 60° C. overnight. More N-bromosuccinimide (0.250 g, 1.40 mmol) was added to the reaction mixture and the mixture was again heated at 60° C. overnight, after which time more N-bromosuccinimide (0.250 g, 1.40 mmol) was added and the mixture was again heated at 60° C. overnight. The reaction mixture was diluted with saturated aqueous sodium carbonate solution, and the aqueous mixture was extracted with dichloromethane. The combined organic layers were dried over magnesium sulfate, filtered and concentrated under reduced pressure. The resulting material was purified by medium pressure liquid chromatography on silica gel (0 to 100% gradient of ethyl acetate in hexanes as eluant) to provide the title compound, a compound of the present invention, as a solid (0.36 g).

¹H NMR (CDCl₃): δ 7.19 (m, 2H), 7.07 (m, 2H), 6.95 (m, 1H), 6.79 (m, 1H), 3.83 (s, 3H).

### Example 16

### Preparation of 5-(2,6-difluoro-3-methoxyphenyl)-1-(4-fluorophenyl)-2,4-dimethyl-1H-imidazole (Compound 549)

A mixture of 2,4-dibromo-5-(2,6-difluoro-3-methoxyphenyl)-1-(4-fluorophenyl)-1H-imidazole (i.e. the product of Example 15) (0.314 g, 0.68 mmol), trimethylboroxine (0.085 g, 0.490 mmol), cesium carbonate (0.665 g, 2.04 mmol) and dichlorobis(triphenyl-phosphine)palladium (0.049 g, 0.07 mmol) in dioxane (5 mL) and water (2 drops) was heated at reflux for 3 days. More trimethylboroxine (0.085 g, 0.68 mmol) and dichlorobis (triphenylphosphine)palladium (0.049 g, 0.70 mmol) were added at the reaction mixture, and the mixture was heated to reflux overnight. The reaction mixture was concentrated under reduced pressure and the resulting material was purified by medium pressure liquid chromatography on silica gel (0 to 100% gradient of ethyl acetate in hexanes as eluant) to provide the title compound, a compound of the present invention, as a solid (0.097 g).

¹H NMR (CDCl₃): δ 7.11 (m, 2H), 7.02 (m, 2H), 6.84 (m, 1H), 6.73 (m, 1H), 3.81 (s, 3H), 2.29 (s, 3H), 2.16 (s, 3H).

### Example 17

### Preparation of 4-chloro-1-[3-(difluoromethoxy)phenyl]-5-(2,6-difluoro-3-methoxyphenyl)-2-ethenyl-1H-imidazole (Compound 583)

A mixture of 2-bromo-4-chloro-1-[3-(difluoromethoxy)phenyl]-5-(2,6-difluoro-3-methoxyphenyl)-1H-imidazole (prepared by a procedure analogous to Example 12), 2,4,6-trivinylcyclotriboroxane pyridine complex (0.103 g, 0.43 mmol), cesium carbonate (0.420 g, 1.29 mmol) and dichlorobis(triphenylphosphine)palladium (0.028 g, 0.040 mmol) in dioxane (5 mL) and water (2 drops) was heated at reflux for 2.5 days. The reaction mixture was concentrated under reduced pressure, and the resulting material was purified by medium pressure liquid chromatography on silica gel (0 to 100% gradient of ethyl acetate in hexanes as eluant) to provide the title compound, a compound of the present invention, as a solid (30 mg).

¹H NMR (CDCl₃): δ 7.37 (m, 1H), 7.16 (m, 1H), 7.05 (d, 1H), 6.93 (m, 2H), 6.78 (m, 1H), 6.35 (m, 3H), 5.43 (m, 1H), 3.82 (s, 3H).

### Example 18

### Preparation of 4-chloro-1-(4-chlorophenyl)-5-(2,3,6-trifluorophenyl)-1H-imidazole-2-carbonitrile (Compound 57)

A mixture of 2-bromo-4-chloro-1-(4-chlorophenyl)-5-(2,3,6-trifluorophenyl)-1H-imidazole (prepared by a procedure analogous to Example 12), zinc cyanide (0.033 g, 0.280 mmol), dichloro[1,1′-bis(diphenylphosphino)ferrocene]palladium(II) dichloromethane complex (1:1) (0.016 g, 0.02 mmol) and tetramethylethylenediamine (0.01 g, 0.095 mmol) in N,N-dimethylformamide (3 mL) was heated at 60° C. in a Biotage Initiator™ microwave apparatus for 200 seconds. The reaction mixture was concentrated under reduced pressure, and the resulting material was purified by medium pressure liquid chromatography on silica gel (0 to 100% gradient of ethyl acetate in hexanes as eluant) to provide the title compound, a compound of the present invention, as a solid (0.030 g) melting at 147-149° C.

¹H NMR (CDCl₃): δ 7.45 (m, 2H), 7.28 (m, 1H), 7.21 (m, 2H), 6.89 (m, 1H).

### Example 19

### Preparation of 4-chloro-5-(2,6-difluorophenyl)-2-ethynyl-1-(3-fluorophenyl)-1H-imidazole (Compound 47)

### Step A: Preparation of 4-chloro-5-(2,6-difluorophenyl)-1-(3-fluorophenyl)-2-[2-(trimethylsilyl)ethynyl]-1H-imidazole

A mixture of 2-bromo-4-chloro-5-(2,6-difluorophenyl)-1-(3-fluorophenyl)-1H-imidazole (prepared by a procedure analogous to Example 12) (0.823 g, 2.10 mmol), dichlorobis(triphenylphosphine)palladium (0.147 g, 0.21 mmol), copper(I) iodide (0.081 g, 0.74 mmol) in triethylamine (15 mL) was stirred for 5 minutes while nitrogen gas flowed through a syringe needle below the surface of the reaction mixture. Ethynyltrimethyl silane (0.216 g, 2.2 mmol) was added to the reaction mixture, stirring was continued for 2 h, and then the mixture was heated at reflux overnight. More dichlorobis(triphenylphosphine)palladium (0.147 g, 0.21 mmol) and ethynyltrimethylsilane (0.216 g, 2.2 mmol) were added to the reaction mixture, and the mixture was heated at reflux for 4 h. The reaction mixture was diluted with saturated aqueous sodium carbonate solution and extracted with ethyl acetate, and the combined organic layers were washed with saturated aqueous ethylenediaminetetraacetic acid, dried over magnesium sulfate, filtered and concentrated under reduced pressure. The resulting material was purified by medium pressure liquid chromatography on silica gel (0 to 100% gradient of ethyl acetate in hexanes as eluant) to provide the title compound as a solid (0.244 g).

¹H NMR (CDCl₃): δ 7.34 (m, 2H), 7.05 (m, 3H), 6.89 (m, 2H), 0.13 (s, 9H).

### Step B Preparation of 4-chloro-5-(2,6-difluorophenyl)-2-ethynyl-1-(3-fluorophenyl)-1H-imidazole

A mixture of 2-bromo-4-chloro-5-(2,6-difluorophenyl)-1-(3-fluorophenyl)-1H-imidazole (i.e. the product of Step A) (0.231 mg, 0.570 mmol) in a solution of sodium hydroxide and methanol (1%, w/w, 2 mL) was stirred for 3 h at room temperature. The reaction mixture was diluted with ethyl acetate and saturated aqueous ammonium chloride solution, the layers were separated, and the aqueous layer was extracted with ethyl acetate. The combined organic layers were dried over magnesium sulfate, filtered and concentrated under reduced pressure to provide the title compound, a compound of the present invention, as a solid (0.135 g).

¹H NMR (CDCl₃): δ 7.35 (m, 2H), 7.11 (m, 1H), 7.00 (m, 2H), 6.89 (m, 2H), 3.17 (s, 1H).

### Example 20

### Preparation of 4-chloro-5-(2,6-difluoro-4-methoxyphenyl)-1-(4-fluorophenyl)-1H-imidazole-2-carboxaldehyde (Compound 276)

To a stirred mixture of 4-chloro-5-(2,6-difluoro-4-methoxyphenyl)-1-(4-fluorophenyl)-1H-imidazole (prepared by a procedure analogous to Example 11) (1.35 g, 4.0 mmol) in diethyl ether (10 mL) at 0° C. was added lithium diisopropylamide (2 M in tetrahydrofuran, 2.2 mL, 4.4 mmol). The reaction mixture was stirred for 1 h at 0° C., and then N,N-dimethylformamide (0.47 mL, 6.0 mmol) was added. After an additional 1 h of stirring at 0° C., the reaction mixture was allowed to warm to room temperature. After 1 h, the reaction mixture was diluted with aqueous citric acid solution (20%, 30 mL) and extracted with diethyl ether (100 mL). The organic layers were dried over magnesium sulfate, filtered and concentrated under reduced pressure. The resulting material was purified by flash column chromatography on silica gel (0 to 20% gradient of ethyl acetate in n-butyl chloride as eluant) to provide the title compound, a compound of the present invention, as a pale-yellow solid (0.397 g).

¹H NMR (CDCl₃): δ 9.71 (s, 1H), 7.19-7.17 (m, 2H), 7.06 (t, J=7.5 Hz, 2H), 6.44 (m, 1H), 6.42 (s, 1H), 3.78 (s, 3H).

ESI MS m/z 367 (M+1).

### Example 21

### Preparation of 4-chloro-5-(2,6-difluoro-4-methoxyphenyl)-1-(4-fluorophenyl)-1H-imidazole-2-methanol (Compound 298)

To a mixture of 4-chloro-5-(2,6-difluoro-4-methoxyphenyl)-1-(4-fluorophenyl)-1H-imidazole-2-carboxaldehyde (i.e. the product of Example 20) in methanol (10 mL) was added sodium borohydride (1.10 g, 2.64 mmol). After 1 h, water (25 mL) was added to the reaction mixture, and the aqueous mixture was extracted with diethyl ether (50 mL), dried over magnesium sulfate, filtered and concentrated under reduced pressure. The resulting material was purified by flash column chromatography on silica gel (0 to 30% gradient of ethyl acetate in hexanes as eluant) to provide the title compound, a compound of the present invention, as an off-white solid (0.156 g).

¹H NMR (CDCl₃): δ 7.26-7.25 (m, 2H), 7.07 (t, J=7.5 Hz, 2H), 6.42 (s, 1H), 6.39 (s, 1H), 4.54 (d, J=3 Hz, 2H), 4.13 (t, J=6 Hz, 1H), 3.77 (s, 3H).

### Example 22

### Preparation of 4-chloro-5-(2,6-difluoro-4-methoxyphenyl)-2-(fluoromethyl)-1-(4-fluorophenyl)-1H-imidazole (Compound 331)

To a mixture of 4-chloro-5-(2,6-difluoro-4-methoxyphenyl)-1-(4-fluorophenyl)-1H-imidazole-2-methanol (i.e. the product of Example 21) in dichloromethane (2 mL) was added diethylaminosulfur trifluoride (60 μL, 0.45 mmol). After 2 h, the reaction mixture was diluted with water, extracted with dichloromethane (100 mL), dried over magnesium sulfate, filtered and concentrated under reduced pressure. The resulting material was purified by flash column chromatography on silica gel (0 to 20% gradient of ethyl acetate in hexanes as eluant) to provide the title compound, a compound of the present invention, as a white solid (0.031 g).

¹H NMR (CDCl₃): δ 7.25-7.23 (m, 2H), 7.08 (t, J=6 Hz, 2H), 6.43 (s, 1H), 6.41 (s, 1H), 5.25 (s, 1H), 5.13 (s, 1H), 3.78 (s, 3H).

### Example 23

### Preparation of 4-chloro-5-(2,6-difluoro-4-methoxyphenyl)-1-(4-fluorophenyl)-1H-imidazole-2-carboxaldehyde oxime (Compound 349)

To a mixture of 4-chloro-5-(2,6-difluoro-4-methoxyphenyl)-1-(4-fluorophenyl)-1H-imidazole-2-carboxaldehyde (i.e. the product of Example 20) in methanol (2 mL) was added a solution of hydroxylamine hydrochloride (0.165 g, 2.4 mmol) and sodium carbonate (0.127 g, 1.2 mmol) in water (1 mL). The reaction mixture was heated at 70° C. for 1 h and then allowed to cool to room temperature. After 48 h, the reaction mixture was filtered, and the solid collected was washed with methanol (2 mL) to provide the title compound, a compound of the present invention, as a shiny-white solid (0.148 g).

¹H NMR (CDCl₃): δ 11.0 (br s, 1H), 7.42 (s, 1H), 6.82-6.79 (t, 2H), 6.68 (t, J=6 Hz, 2H), 6.07 (s, 1H), 6.05 (s, 1H), 3.35 (s, 3H).

### Example 24

### Preparation of 4-bromo-2-(bromoethyl)-1-(4-chlorophenyl)-5-(2,6-difluorophenyl)-1H-imidazole (Compound 475)

A mixture of 2-bromo-1-(4-chlorophenyl)-5-(2,6-difluorophenyl)-2-methyl-1H-imidazole (i.e. the product of Example 6) (1.00 g, 2.6 mmol), N-bromosuccinimide (0.510 g, 2.87 mmol) and 2,2′-azobis(2-methylpropionitrile) (0.021 g, 130 mmol) in carbon tetrachloride (8 mL) was heated at reflux overnight. The reaction mixture was concentrated under reduced pressure, and the resulting material was purified by medium pressure liquid chromatography on silica gel (0 to 100% gradient of ethyl acetate in hexanes as eluant) to provide the title compound, a compound of the present invention, as a solid (0.940 g).

¹H NMR (CDCl₃): δ 7.35 (m, 3H), 7.24 (m, 2H), 6.87 (m, 2H), 4.36 (s, 2H).

### Example 25

### Preparation of 4-bromo-1-(4-chlorophenyl)-5-(2,6-difluorophenyl)-1H-imidazole-2-acetonitrile (Compound 509)

A mixture of 4-bromo-2-(bromoethyl)-1-(4-chlorophenyl)-5-(2,6-difluorophenyl)-1H-imidazole (i.e. the product of Example 24) (1.00 g, 2.6 mmol), potassium cyanide (0.105 g, 1.62 mmol) and 18-crown-6 (0.057 g, 0.216 mmol) in acetonitrile (3 mL) was heated at 40° C. overnight. The reaction mixture was concentrated under reduced pressure, and the resulting material was purified by medium pressure liquid chromatography on silica gel (0 to 100% gradient of ethyl acetate in hexanes as eluant) to provide the title compound, a compound of the present invention, as a solid (0.105 g).

¹H NMR (CDCl₃): δ 7.38 (m, 3H), 7.18 (m, 2H), 6.89 (m, 2H), 3.76 (s, 2H).

### Example 26

### Preparation of methyl 4-bromo-1-(4-chlorophenyl)-5-(2,6-difluorophenyl)-1H-imidazole-2-acetate (Compound 544)

To a mixture of thionyl chloride (0.232 mL) in methanol (5 mL) was added 4-bromo-1-(4-chlorophenyl)-5-(2,6-difluorophenyl)-1H-imidazole-2-acetonitrile (i.e. the product of Example 25) (0.650 g 1.59 mmol). The reaction mixture was heated at reflux overnight, then more thionyl chloride (0.5 mL) was added, and the mixture was heated at reflux for an additional 8 h. The reaction mixture was diluted with ethyl acetate and washed with water (2×), and the ethyl acetate mixture was neutralized by the addition of saturated aqueous sodium bicarbonate solution. The aqueous mixture was extracted with ethyl acetate (2×), and the combined organic layers were washed with saturated aqueous sodium bicarbonate solution, dried over magnesium sulfate, filtered and concentrated under reduced pressure. The resulting material was purified by medium pressure liquid chromatography on silica gel (0 to 100% gradient of ethyl acetate in hexanes as eluant) to provide the title compound, a compound of the present invention, as a solid (0.470 g).

¹H NMR (CDCl₃): δ 7.32 (m, 3H), 7.15 (m, 2H), 6.86 (dd, 2H), 3.70 (s, 2H), 3.66 (s, 3H).

### Example 27

### Preparation of 4-bromo-1-(4-chlorophenyl)-5-(2,6-difluorophenyl)-1H-imidazole-2-acetic acid (Compound 570)

A mixture of methyl 4-bromo-1-(4-chlorophenyl)-5-(2,6-difluorophenyl)1H-imidazole-2-acetate (i.e. the product of Example 26) (0.100 g, 0.226 mmol) and lithium hydroxide monohydrate (0.019 g, 0.453 mmol) in tetrahydrofuran and water (1:1, 2 mL) was stirred for 20 minutes, and then hydrochloric acid (1 N, 0.45 mL) was added. The reaction mixture was extracted with ethyl acetate (3×), and the combined organic layers were dried over magnesium sulfate, filtered and concentrated under reduced pressure. The resulting material was purified by medium pressure liquid chromatography on silica gel (0 to 100% gradient of ethyl acetate in hexanes as eluant) to provide the title compound, a compound of the present invention, as a solid (0.037 g).

¹H NMR (CD₃COCD₃): δ 7.51 (m, 3H), 7.35 (m, 2H), 7.05 (m, 2H), 3.77 (s, 2H).

### Example 28

### Preparation of 4-bromo-1-(4-chlorophenyl)-5-(2,6-difluorophenyl)-N-methyl-1H-imidazole-2-acetamide (Compound 571)

A mixture of 4-bromo-1-(4-chlorophenyl)-5-(2,6-difluorophenyl)-1H-imidazole-2-acetic acid (i.e. the product of Example 27) (0.217 g, 0.507 mmol), methylamine (2 M in tetrahydrofuran, 0.505 mL, 1.01 mmol) and N-(3-dimethylaminopropyl)-N-ethylcarbodiimide hydrochloride (0.117 g, 0.609 mmol) in pyridine (4 mL) and dichloromethane (3 mL) were stirred at room temperature overnight. More methylamine (2 M in tetrahydrofuran, 0.505 mL, 1.01 mmol), N-(3-dimethylaminopropyl)-N-ethylcarbodiimide hydrochloride (0.117 g, 0.609 mmol) and pyridine (1 mL) were added to the reaction mixture and stirring was continued for 4 h. The reaction mixture was diluted with ethyl acetate and then washed with water (3×) and saturated aqueous sodium chloride solution. The organic layer was dried over magnesium sulfate, filtered and concentrated under reduced pressure. The resulting material was purified by medium pressure liquid chromatography on silica gel (0 to 100% gradient of ethyl acetate in hexanes as eluant) to provide the title compound, a compound of the present invention, as a solid (0.063 g).

¹H NMR (CDCl₃): δ 7.52 (br s, 1H), 7.34 (m, 3H), 7.11 (m, 2H), 6.88 (m, 2H), 3.54 (s, 2H), 2.87 (d, 3H).

### Example 29

### Preparation of 3-[4-chloro-1-(4-chlorophenyl)-1H-imidazol-5-yl]-2,4-difluorobenzonitrile (Compound 386)

A mixture of 5-(3-bromo-2,6-difluorophenyl)-4-chloro-1-(4-chlorophenyl)-1H-imidazole (prepared a procedure analogous to Example 11) (0.440 g, 0.490 mmol), zinc cyanide (0.058 g, 0.490 mmol), dichloro[1,1′-bis(diphenylphosphino)ferrocene]palladium(II) dichloromethane complex (1:1) (0.044 g, 0.0545 mmol) and N,N,N′,N′-tetramethylethylenediamine (0.022 g, 0.218 mmol) in dimethylacetamide (3 mL) was heated at 200° C. in a Biotage Initiator™ microwave apparatus for 5 minutes. The reaction mixture was concentrated under reduced pressure, and the resulting material was purified by medium pressure liquid chromatography on silica gel (0 to 100% gradient of ethyl acetate in hexanes as eluant) to provide the title compound, a compound of the present invention, as a solid (0.217 g).

¹H NMR (CDCl₃): δ 7.72 (s, 1H), 7.69 (m, 1H), 7.38 (m, 2H), 7.07 (m, 3H).

### Example 30

### Preparation of 3-[2-bromo-4-chloro-1-(4-chlorophenyl)-1H-imidazol-5-yl]-2,4-difluorobenzonitrile (Compound 419)

A mixture of 3-[4-chloro-1-(4-chlorophenyl)-1H-imidazol-5-yl]-2,4-difluorobenzonitrile (i.e. the product of Example 29) (0.217 g, 0.62 mmol) and N-bromosuccinimide (0.165 g, 0.930 mmol) in dimethylformamide (4 mL) was heated at 60° C. overnight. More N-bromosuccinimide (0.386 g, 2.17 mmol) was added portionwise to the reaction mixture, and the mixture was heated at 60° C. overnight again. The reaction mixture was diluted with ethyl acetate, washed with saturated aqueous sodium chloride solution, dried over magnesium sulfate, filtered and concentrated under reduced pressure. The resulting material was purified by medium pressure liquid chromatography on silica gel (0 to 100% gradient of ethyl acetate in hexanes as eluant) to provide the title compound, a compound of the present invention, as a solid (0.196 g).

¹H NMR (CDCl₃): δ 7.67 (m, 1H), 7.40 (m, 2H), 7.13 (m, 2H), 7.03 (m, 1H).

### Example 31

### Preparation of 3-[4-chloro-1-(4-chlorophenyl)-2-methyl-1H-imidazol-5-yl]-2,4-difluorobenzonitrile (Compound 430)

A mixture of 3-[2-bromo-4-chloro-1-(4-chlorophenyl)-1H-imidazol-5-yl]-2,4-difluorobenzonitrile (i.e. the product of Example 30) (0.150 g, 0.350 mmol), trimethylboroxine (0.088 g, 0.700 mmol), cesium carbonate (0.342 g, 1.05 mmol) and dichlorobis(triphenylphosphine)palladium (0.025 g, 0.035 mmol) in dioxane (4 mL) and water (2 drops) was heated at reflux for 3 h. The reaction mixture was concentrated under reduced pressure, and the resulting material was purified by medium pressure liquid chromatography on silica gel (0 to 100% gradient of ethyl acetate in hexanes as eluant) to provide the title compound, a compound of the present invention, as a solid (0.088 g).

¹H NMR (CDCl₃): δ 7.65 (m, 1H), 7.39 (m, 2H), 7.09 (m, 2H), 7.01 (m, 1H), 2.32 (s, 3H).

### Example 32

### Preparation of 4-[4-chloro-2-methyl-1-(4-methylphenyl)-1H-imidazol-5-yl]-3,5-difluorophenol (Compound 624)

To a stirred mixture of 4-chloro-5-(2,6-difluoro-4-methoxyphenyl)-2-methyl-1-(4-methylphenyl)-1H-imidazole (prepared by a procedure analogous to Example 13) (0.500 g, 1.43 mmol) in dichloromethane (10 mL) at −78° C. was added tribromoborane (1 M in dichloromethane, 4.3 mL, 4.30 mmol). The reaction mixture was allowed to warm to room temperature, and stirred overnight. More tribromoborane (1 M in dichloromethane, 1.4 mL, 1.40 mmol) was added to the reaction mixture at room temperature, and stirring was continued for 4 h. Hydrochloric acid (1 N, 8.0 mL) was added to the reaction mixture, and then the aqueous mixture was brought to a basic pH by the addition of saturated aqueous sodium carbonate solution. The aqueous mixture was extracted with ethyl acetate, and the extract was dried over magnesium sulfate, filtered and concentrated under reduced pressure. The resulting material was purified by medium pressure liquid chromatography on silica gel (0 to 100% gradient of ethyl acetate in hexanes as eluant) to provide the title compound, a compound of the present invention, as a solid (0.41 g).

¹H NMR (CDCl₃): δ 10.68 (s, 1H), 7.24 (d, 2H), 7.09 (d, 2H), 6.43 (m, 2H), 2.31 (s, 3H), 2.18 (s, 3H).

### Example 33

### Preparation of 3-[4-[4-chloro-2-methyl-1-(4-methylphenyl)-1H-imidazol-5-yl]-3,5-difluorophenoxy]-N-methyl-1-propanamide hydrochloride (Compound 734)

### Step A: Preparation of phenylmethyl N-[3-[4-[4-chloro-2-methyl-1-(4-methylphenyl)-1H-imidazol-5-yl]-3,5-difluorophenoxy]propyl]-N-methylcarbamate

A mixture of 4-[4-chloro-2-methyl-1-(4-methylphenyl)-1H-imidazol-5-yl]-3,5-difluorophenol (i.e. the product of Example 32) (0.200 g, 0.598 mmol) and 4 A molecular sieves (1.55 g) in N,N-dimethylformamide (3 mL) was stirred at room temperature for 3 h, and then a solution of phenylmethyl N-(3-chloropropyl)-N-methylcarbamate (prepared by the method described in PCT Publication WO 2007/149448) (0.434 g, 1.80 mmol) and tetrabutylammonium iodide (0.044 g, 0.120 mmol) in N,N-dimethylformamide (1 mL) was added. After 15 minutes, cesium carbonate (0.584 g, 1.80 mmol) was added to the reaction mixture. After 15 minutes, the reaction mixture was heated at 75° C. for 2 h. The reaction mixture was filtered, and the filtrate was concentrated under reduced pressure. The resulting material was purified by medium pressure liquid chromatography on silica gel (0 to 100% gradient of ethyl acetate in hexanes as eluant) to provide the title compound as a solid (210 mg).

ESI MS m/z 541 (M+1).

### Step B Preparation of 3-[4-[4-chloro-2-methyl-1-(4-methylphenyl)-1H-imidazol-5-yl]-3,5-difluorophenoxy]-N-methyl-1-propanamide hydrochloride

A mixture of phenylmethyl N-[3-[4-[4-chloro-2-methyl-1-(4-methylphenyl)-1H-imidazol-5-yl]-3,5-difluorophenoxy]propyl]-N-methylcarbamate (i.e. the product of Step A) (0.197 g, 0.365 mmol), hydrochloric acid (2 M in diethyl ether, 1 mL) and methanol (30 mL) was purged with nitrogen for 30 minutes, and then palladium on carbon (10%, 0.058 g, 0.0547 mmol) was added and the nitrogen purge was maintained for an additional 15 minutes. After 15 minutes, the nitrogen purge was stopped and a balloon filled with hydrogen was connected to the reaction flask, and the reaction mixture was stirred at room temperature for 3 h. The reaction mixture was purged with nitrogen and the palladium on carbon catalyst was removed by filtering through a bed of sand and Celite® (diatomaceous filter aid) on a sintered glass frit funnel. The filtrate was concentrated under reduced pressure to provide the title compound, a compound of the present invention, as a solid (0.140 g).

¹H NMR (DMSO-d₆): δ 8.90 (br s, 2H), 7.25 (d, 2H), 7.11 (d, 2H), 6.76 (d, 2H), 4.08 (t, 2H), 2.98 (m, 2H), 2.54 (m, 3H), 2.31 (s, 3H), 2.19 (s, 3H), 2.04 (d, 2H).

### Example 34

### Preparation of 5-[4-chloro-5-(2,6-difluoro-4-methoxyphenyl)-1H-imidazol-1-yl]-2-(trifluoromethyl)pyridine

### Step A: Preparation of N-[(2,6-difluoro-4-methoxyphenyl)methylene]-6-(trifluoromethyl)-3-pyridinamine

A mixture of 6-(trifluoromethyl)-3-pyridinamine (5.00 g, 30.8 mmol) and 2,6-difluoro-4-methoxybenzaldehyde (5.30 g, 30.8 mmol) in toluene (100 mL) was heated to reflux for 2.5 days. Then the reaction mixture was concentrated under reduced pressure to provide the title compound as a solid (9.9 g).

¹H NMR (CDCl₃): δ 8.56 (s, 1H), 8.52 (d, 1H), 7.71 (d, 1H), 7.60 (m, 1H), 6.57 (m, 2H), 3.88 (s, 3H).

### Step B Preparation of 5-[5-(2,6-difluoro-4-methoxyphenyl)-1H-imidazol-1-yl]-2-(trifluoromethyl)pyridine

A mixture of N-[(2,6-difluoro-4-methoxyphenyl)methylene]-6-(trifluoromethyl)-3-pyridinamine (i.e. the product of Step A), 1-[(1-isocyanoethyl)sulfonyl]-4-methylbenzene (4.6 g, 23.7 mmol) and potassium carbonate (4.4 g, 31.6 mmol) in dimethoxyethane (20 mL) and methanol (20 mL) was heated at 75° C. overnight. The reaction mixture was concentrated under reduced pressure, and the resulting material was purified by medium pressure liquid chromatography on silica gel (0 to 100% gradient of ethyl acetate in hexanes as eluant) to provide the title compound as a solid (4.63 g).

¹H NMR (CDCl₃): δ 8.59 (m, 1H), 7.86 (s, 1H), 7.71 (m, 2H), 7.33 (m, 1H), 6.46 (m, 2H), 3.80 (s, 3H).

### Step C Preparation of 5-[4-chloro-5-(2,6-difluoro-4-methoxyphenyl)-1H-imidazol-1-yl]-2-(trifluoromethyl)pyridine

A mixture of 5-[5-(2,6-difluoro-4-methoxyphenyl)-1H-imidazol-1-yl]-2-(trifluoromethyl)pyridine (i.e. the product of Step B) (1.50 g, 4.20 mmol), N-chlorosuccinimide (0.56 g, 4.2 mmol) and 2,2′-azobis(2-methylpropionitrile) (0.038 g, 0.230 mmol) in carbon tetrachloride (8 mL) was heated at 65° C. for 3.5 days. The reaction mixture was diluted with saturated aqueous sodium carbonate solution and extracted with dichloromethane. The combined organic layers were dried over magnesium sulfate, filtered and concentrated under reduced pressure. The resulting material was purified by medium pressure liquid chromatography on silica gel (0 to 100% gradient of ethyl acetate in hexanes as eluant) to provide the title compound, a compound of the present invention, as a solid (0.650 g).

¹H NMR (CDCl₃): δ 8.59 (m, 1H), 7.73 (m, 3H), 6.49 (m, 2H), 3.82 (s, 3H).

### Example 35

### Preparation of 5-[2-bromo-4-chloro-5-(2,6-difluoro-4-methoxyphenyl)-1H-imidazol-1-yl]-2-(trifluoromethyl)pyridine (Compound 707)

A mixture of 5-[4-chloro-5-(2,6-difluoro-4-methoxyphenyl)-1H-imidazol-1-yl]-2-(trifluoromethyl)pyridine (i.e. the product of Step C of Example 34) (0.650 g, 1.70 mmol) and N-bromosuccinimide (0.356 g, 2.0 mmol) in N,N-dimethylformamide (10 mL) was heated at 65° C. overnight. More N-bromosuccinimide (0.195 g, 1.1 mmol) was added to the reaction mixture, and the mixture was heated at 65° C. for 2.5 days. The reaction mixture was diluted with saturated sodium carbonate solution and extracted with dichloromethane. The combined organic layers were dried over magnesium sulfate, filtered and concentrated under reduced pressure. The resulting material was purified by medium pressure liquid chromatography on silica gel (0 to 100% gradient of ethyl acetate in hexanes as eluant) to provide the title compound, a compound of the present invention, as a solid (0.108 g).

¹H NMR (CDCl₃): δ 8.58 (s, 1H), 7.78 (m, 2H), 6.43 (m, 2H), 3.79 (s, 3H).

### Example 36

### Preparation of 5-[4-chloro-5-(2,6-difluoro-4-methoxyphenyl)-2-methyl-1H-imidazol-1-yl]-2-(trifluoromethyl)pyridine (Compound 713)

A mixture of 5-[2-bromo-4-chloro-5-(2,6-difluoro-4-methoxyphenyl)-1H-imidazol-1-yl]-2-(trifluoromethyl)pyridine (i.e. the product of Example 35) (0.093 g, 0.20 mmol), trimethylboroxine (0.025 g, 0.20 mmol), cesium carbonate (0.195 g, 0.60 mmol) and dichlorobis(triphenylphosphine)palladium (0.014 g, 0.20 mmol) in dioxane (3 mL) and water (1 drop) was heated to reflux overnight. Then the reaction mixture was concentrated under reduced pressure, and the resulting material was purified by medium pressure liquid chromatography on silica gel (0 to 100% gradient of ethyl acetate in hexanes as eluant) to provide the title compound, a compound of the present invention, as a solid (0.021 g).

¹H NMR (CDCl₃): δ 8.55 (s, 1H), 7.75 (m, 1H), 7.69 (m, 1H), 6.42 (m, 2H), 3.78 (s, 3H), 2.35 (s, 3H).

### Example 37

### Preparation of 2-chloro-5-[1-(2,6-difluoro-4-methoxyphenyl)-4-methyl-1H-imidazol-5-yl]pyridine (Compound 685)

### Step A: Preparation of N-[(6-chloro-3-pyridinyl)methylene]-2,6-difluoro-4-methoxybenzenamine

To a mixture of 2,6-difluoro-4-methoxybenzenamine (0.98 g, 6.9 mmol) in toluene (20 mL) was added 6-chloro-3-pyridinecarboxaldehyde (1.0 g, 6.3 mmol). The reaction mixture was heated at reflux with the use of a Dean-Stark trap for azeotropic removal of water. After 16 h the reaction mixture was cooled to room temperature and concentrated under reduced pressure. The resulting material was dried in a vacuum oven at 55° C. overnight to provide the title compound as a light brown solid (1.66 g).

¹H NMR (CDCl₃): δ 8.74 (s, 1H), 8.73 (d, J=2.44 Hz, 1H), 8.32 (dd, J=2.20 Hz, J=8.29 Hz, 1H), 7.44 (d, J=8.29 Hz, 1H), 6.59-6.52 (m, 2H), 3.82 (s, 3H).

¹F NMR (CDCl₃): δ-121.48 to 121.40 (m, 2F).

### Step B Preparation of 2-chloro-5-[1-(2,6-difluoro-4-methoxyphenyl)-4-methyl-1H-imidazol-5-yl]pyridine

To a mixture of N-[(6-chloro-3-pyridinyl)methylene]-2,6-difluoro-4-methoxybenzenamine (i.e. the product of Step A) (1.66 g, 5.9 mmol) in tetrahydrofuran (15 mL) was added p-toluenesulfonylmethyl isocyanide (1.35 g, 6.5 mmol) and potassium tert-butoxide (0.86 g, 7.7 mmol). The reaction mixture was heated at 85° C. for 4 h, cooled, and concentrated. The resulting material was diluted with ethyl acetate and washed with saturated aqueous sodium chloride solution. The organic layer was dried over magnesium sulfate, filtered and concentrated. The resulting material was purified by medium pressure liquid chromatography on silica gel (ethyl acetate in hexanes), and then triturated with n-butyl chloride-hexanes as eluant to provide the title compound, a compound of the present invention, as a tan solid (0.50 g) melting at 117-118° C.

¹H NMR (CDCl₃): δ 8.16 (d, J=2.44 Hz, 1H), 7.55 (s, 1H), 7.44 (dd, J=2.68 and 8.29 Hz, 1H), 7.28 (dd, J=0.49 and 8.29 Hz, 1H), 6.54-6.48 (m, 2H), 3.81 (s, 3H), 2.33 (s, 3H).

¹FNMR (CDCl₃): δ-118.21 to -118.15 (m, 2F).

### Example 38

### Preparation of 2-chloro-5-[2-chloro-1-(2,6-difluoro-4-methoxyphenyl)-4-methyl-1H-imidazol-5-yl]pyridine (Compound 696)

To a mixture of 2-chloro-5-[1-(2,6-difluoro-4-methoxyphenyl)-4-methyl-1H-imidazol-5-yl]pyridine (i.e. the product of Step B of Example 37) (0.150 g, 0.45 mmol) in N,N-dimethylformamide (2.0 mL) was added N-chlorosuccinimide (0.066 g, 0.49 mmol), and the reaction mixture was heated at 60° C. After 30 minutes, the reaction mixture was cooled to room temperature, diluted with ethyl acetate (40 mL), and washed with water and saturated aqueous sodium chloride solution. The organic layer was dried over magnesium sulfate, filtered and concentrated under reduced pressure. The resulting material was purified by medium pressure liquid chromatography on silica gel (ethyl acetate in hexanes as eluant) to provide the title compound, a compound of the present invention, (0.108 g) as an off-white solid melting at 94-95° C.

¹H NMR (CDCl₃): δ 8.17 (d, J=2.20 Hz, 1H), 7.43 (dd, J=8.29 Hz, J=2.44 Hz, 1H), 7.26 (d, J=8.54 Hz, 1H), 6.53 (d, J=9.02 Hz, 2H), 3.82 (s, 3H), 2.28 (s, 3H).

### Example 39

### Preparation of 3-bromo-5-(4-chlorophenyl)-4-(2,6-difluorophenyl)-1-methyl-1H-pyrazole (Compound 125)

### Step A: Preparation of 1-(4-chlorophenyl)-2-(2,6-difluorophenyl)ethanone

A mixture of 2,6-difluorophenylacetic acid (5.63 g, 31.1 mmol) and thionyl chloride (4.5 mL) in toluene was heated at reflux for 4 h, after which time the reaction mixture was allowed to cool to room temperature and stirred for 1 h. The reaction mixture was concentrated under reduced pressure, diluted with carbon tetrachloride (50 mL), and again concentrated under reduced pressure. To a stirred mixture of the resulting material in chlorobenzene (17.5 mL) was added aluminum chloride (5 g) portionwise such that the reaction temperature was maintained at about 30° C. Upon completion of the addition, the reaction mixture was heated at 50° to 70° C. for 2 h, and then stirred at room temperature overnight. The reaction mixture was poured portionwise into a slurry of ice/hydrochloric acid (1 N), extracted with toluene and concentrated under reduced pressure. The resulting material was diluted with methanol (50 mL), concentrated under reduced pressure, and then partitioned between ethyl acetate and aqueous sodium hydroxide solution (1 N). The organic layer was separated, and the aqueous sodium hydroxide layer was extracted with ethyl acetate. The combined organic layers were dried over magnesium sulfate, filtered and concentrated under reduced pressure to provide the title compound as a brown solid (1.75 g).

¹HNMR (CDCl₃) δ 7.98 (d, 2H), 7.47 (d, 2H), 7.35-7.22 (m, 1H), 6.97-6.87 (m, 2H), 4.34 (s, 2H).

### Step B Preparation of 1-(4-chlorophenyl)-2-(2,6-difluorophenyl)-3-(dimethylamino)-2-prop en-1-one

A mixture of 1-(4-chlorophenyl)-2-(2,6-difluorophenyl)ethanone (i.e. the product of Step A) (0.5 g, 1.9 mmol) and N,N-dimethylformamide dimethyl acetal (3.7 mL, 28.0 mmol) in toluene (34 mL) was heated at reflux overnight. The reaction mixture was concentrated under reduced pressure, and the resulting residue was purified by medium pressure liquid chromatography on silica gel (20:80 to 50:50 gradient of ethyl acetate in hexanes as eluant) to provide of the title compound as a yellow solid (0.49 g).

¹H NMR (CDCl₃): δ 7.51 (br s, 1H), 7.39 (d, 2H), 7.28-7.17 (m, 3H), 6.84-6.78 (m, 2H), 2.84 (br s, 6H).

### Step C Preparation of 3-(4-chlorophenyl)-4-(2,6-difluorophenyl)-1H-pyrazole

A mixture of 1-(4-chlorophenyl)-2-(2,6-difluorophenyl)-3-(dimethylamino)-2-propen-1-one (i.e. the product of Step B) (0.43 g, 1.34 mmol) and hydrazine monohydrochloride (0.14 g, 2.0 mmol) in ethanol (20 mL) was heated at reflux overnight. The reaction mixture was concentrated under reduced pressure, and the resulting residue was purified by medium pressure liquid chromatography on silica gel (20:80 to 40:60 gradient of ethyl acetate in hexanes as eluant) to provide of the title compound as a yellow oil (0.40 g).

¹H NMR (CDCl₃): δ 7.70 (s, 1H), 7.34 (d, 2H), 7.30-7.23 (m, 3H), 6.92 (m, 2H).

### Step D Preparation of 3-bromo-5-(4-chlorophenyl)-4-(2,6-difluorophenyl)-1H-pyrazole

To a stirred mixture of 3-(4-chlorophenyl)-4-(2,6-difluorophenyl)-1H-pyrazole (i.e. the product of Step C) (0.40 g, 1.38 mmol) in dichloromethane (10 mL) at 0° C. was added N-bromosuccinimide (0.24 g, 1.38 mmol) portionwise. The reaction mixture was stirred at room temperature overnight and then cooled to 0° C., and more N-bromosuccinimide (0.12 g, 0.69 mmol) was added. After stirring for 6 h at room temperature, the reaction mixture was cooled to 0° C. and more N-bromosuccinimide (0.12 g, 0.69 mmol) was added, and the mixture was stirred at room temperature overnight. The reaction mixture was diluted with water, stirred for 5 minutes, and then extracted with dichloromethane. The organic layer was dried over magnesium sulfate, filtered and concentrated under reduced pressure. The resulting residue was triturated with n-butyl chloride-hexanes and filtered to provide the title compound as a white solid (0.41 g).

¹H NMR (CDCl₃): δ 7.41-7.28 (m, 3H), 7.26 (d, 2H), 7.03-6.90 (m, 2H).

### Step E Preparation of 3-bromo-5-(4-chlorophenyl)-4-(2,6-difluorophenyl)-1-methyl-1H-pyrazole

To a stirred mixture of 3-bromo-5-(4-chlorophenyl)-4-(2,6-difluorophenyl)-1H-pyrazole (i.e. the product of Step D) (0.35 g, 0.95 mmol) in N,N-dimethylformamide (5 mL) at 0° C. was added sodium hydride (60% in mineral oil, 0.04 g, 0.95 mmol) portionwise. The reaction mixture was stirred for 5 minutes, then methyl iodide (0.67 g, 4.7 mmol) was added, and the mixture was allowed to warm to room temperature and stir for 20 minutes. The reaction mixture was poured into water and extracted with ethyl acetate (2×). The combined organic layers were dried over magnesium sulfate, filtered and concentrated under reduced pressure. The resulting residue was purified by medium pressure liquid chromatography on silica gel (3:97 to 12:88 gradient of ethyl acetate in hexanes as eluant) to provide the title compound, a compound of the present invention, as a solid (12.6 mg).

¹H NMR (CDCl₃): δ 7.35 (d, 2H), 7.31-7.23 (m, 1H), 7.17 (d, 2H), 6.91-6.82 (m, 2H), 3.83 (s, 3H).

### Example 40

### Preparation of 5-(2,6-difluorophenyl)-4-(3-fluorophenyl)-1,3-dimethyl-1H-pyrazole (Compound 60)

### Step A: Preparation of 5-(2,6-difluorophenyl)-1,3-dimethyl-1H-pyrazole

To a solution of glacial acetic acid (2.2 mL) was added 1-(2,6-difluorophenyl)-1,3-butanedione (prepared by the method described in Japanese Patent Application Publication JP 2001/048826) (1.0 g, 5.1 mmol) and N-methylhydrazine (0.23 g, 5.1 mmol). The reaction mixture was heated at reflux for 4 h and then concentrated under reduced pressure. The resulting residue was purified by medium pressure liquid chromatography on silica gel (100:0 to 20:80 gradient of ethyl acetate in hexanes as eluant) to provide the title compound as a white solid (0.26 g).

¹H NMR (CDCl₃): δ 7.43-7.37 (m, 1H), 7.06-6.98 (m, 2H), 6.17 (s, 1H), 3.71 (s, 3H), 2.33 (s, 3H).

### Step B Preparation of 5-(2,6-difluorophenyl)-4-iodo-1,3-dimethyl-1H-pyrazole

A mixture of 5-(2,6-difluorophenyl)-1,3-dimethyl-1H-pyrazole (i.e. the product of Step A) (0.20 g, 0.96 mmol) and N-iodosuccinimide (0.22 g, 0.96 mmol) in N,N-dimethylformamide (5 mL) was heated at 90° C. overnight. The reaction mixture was allowed to cool to room temperature and then partitioned between water and ethyl acetate. The layers were separated and the aqueous layer was extracted with ethyl acetate. The combined organic layers were dried over magnesium sulfate, filtered and concentrated under reduced pressure. The resulting residue was purified by medium pressure liquid chromatography on silica gel (5:95 to 20:80 gradient of ethyl acetate in hexanes as eluant) to provide the title compound as a light brown oil (0.22 g).

¹H NMR (CDCl₃): δ 7.55-7.42 (m, 1H), 7.10-7.03 (m, 2H), 3.74 (s, 3H), 2.32 (s, 3H).

### Step C Preparation of 5-(2,6-difluorophenyl)-4-(3-fluorophenyl)-1,3-dimethyl-1H-pyrazole

To toluene (5 mL) was added 5-(2,6-difluorophenyl)-4-iodo-1,3-dimethyl-1H-pyrazole (i.e. the product of Step B) (0.22 g, 0.66 mmol), 3-fluorophenylboronic acid (0.18 g, 1.32 mmol), tris(dibenzylideneacetone)dipalladium(0) (0.02 g, 0.02 mmol), 2-dicyclohexylphosphino-2′,6′-dimethoxybiphenyl (0.04 g, 0.09 mmol), and potassium phosphate (0.43 g, 2.0 mmol), and the mixture was then heated at 100° C. overnight. The reaction mixture was allowed to cool to room temperature, and then concentrated under reduced pressure. The resulting material was purified by medium pressure liquid chromatography on silica gel (5:95 to 30:70 gradient of ethyl acetate in hexanes as eluant) to provide a solid. The solid was triturated with n-butyl chloride, filtered and air-dried to provide the title compound, a compound of the present invention, as an off-white solid (0.08 g).

¹H NMR (CDCl₃): δ 7.45-7.38 (m, 1H), 7.28-7.20 (m, 2H), 7.04-6.79 (m, 4H), 3.71 (s, 3H), 2.36 (s, 3H).

### Example 41

### Preparation of 4-(2-chloro-4-fluorophenyl)-5-[(2,4-difluorophenyl)methyl]-1,3-dimethyl-1H-pyrazole (Compound 313)

### Step A: Preparation of 4-(2-chloro-4-fluorophenyl)-1,3-dimethyl-1H-pyrazol-5-amine

To a stirred mixture of sodium hydride (60% in mineral oil, 3.5 g, 88 mmol) in xylenes (34 mL) at 50° C. was added ethanol (20 mL, 34 mmol) dropwise over about 15 minutes while maintaining an atmosphere of nitrogen. A solution of 2-chloro-4-fluorophenylacetonitrile (4.8 g, 28 mmol) and ethyl acetate (20 mL, 38 mmol) in xylenes (6 mL) was added dropwise over 15 minutes to the reaction mixture, and the mixture was heated at reflux for 2 h, then allowed to cool to room temperature. The reaction mixture was diluted with water (50 mL) and extracted with hexanes (50 mL). The aqueous layer was then acidified to pH 4 with aqueous hydrochloric acid (3 N) solution and extracted with diethyl ether (3×100 mL). The combined diethyl ether layers were washed with saturated aqueous sodium chloride solution, dried over magnesium sulfate, filtered, and concentrated under reduced pressure to provide a tan solid (4.6 g). To a of mixture of the tan solid in ethanol (15 mL) was added acetic acid (3 mL) and methyl hydrazine (1.2 mL, 22 mmol), and the mixture was heated at reflux for 3 h, allowed to cool, and concentrated under reduced pressure. The resulting residue was triturated with ethyl acetate (about 5 mL) and filtered through a glass frit funnel, and the solid collected was air-dried to provide the title compound as a white solid (2.4 g).

¹H NMR (CD₃COCD₃): δ 7.35-7.30 (m, 2H), 7.14 (m, 1H), 4.43 (br s, 2H), 3.60 (s, 3H), 1.94 (s, 3H).

### Step B Preparation of 5-bromo-4-(2-chloro-4-fluorophenyl)-1,3-dimethyl-1H-pyrazole

To a mixture of 4-(2-chloro-4-fluorophenyl)-1,3-dimethyl-1H-pyrazol-5-amine (i.e. the product of Step A) (2.4 g, 10 mmol) in acetonitrile (50 mL) was added copper(II) bromide (3.94 g, 17.7 mmol). The reaction mixture was cooled to about 0° C. with an ice-water bath, and then tent-butyl nitrite (90% technical grade, 2.33 mL, 17.7 mmol) was added dropwise over 5 minutes. The reaction mixture was allowed to warm slowly to room temperature. Aqueous hydrochloric acid solution (1 N, 20 mL) and ethyl acetate (20 mL) were added to the reaction mixture, and then the mixture was filtered through a pad (2 cm) of Celite® (diatomaceous filter aid). The Celite® pad was washed with ethyl acetate (20 mL), the layers were separated, and the organic layer was washed with aqueous hydrochloric acid (1 N) solution and saturated aqueous sodium chloride solution, dried over magnesium sulfate, and concentrated under reduced pressure to provide the title compound as an orange-brown semisolid.

¹H NMR (CDCl₃): δ 7.18-7.25 (m, 2H), 7.04 (m, 1H), 3.89 (s, 3H), 2.14 (s, 3H).

### Step C Preparation of 4-(2-chloro-4-fluorophenyl)-5-[(2,4-difluorophenyl)methyl]-1,3-dimethyl-1H-pyrazole

To a stirred mixture of 5-bromo-4-(2-chloro-4-fluorophenyl)-1,3-dimethyl-1H-pyrazole (i.e. the product of Step B) (0.20 g, 0.66 mmol) in tetrahydrofuran (3 mL) was added dichlorobis(triphenylphosphine)palladium(II) (23 mg, 0.033 mmol) and a solution of 2,4-difluorobenzylzinc chloride (0.5 M in tetrahydrofuran, 2.64 mL, 1.32 mmol). The reaction mixture was heated at reflux for 3 h and then cooled to room temperature and aqueous hydrochloric acid solution (1 N, 3 mL) was added. The aqueous mixture was extracted with ethyl acetate (20 mL), and the organic layer was washed with saturated aqueous sodium chloride solution, dried over magnesium sulfate, and concentrated under reduced pressure to provide an oily material. The oily material was purified by silica gel (5 g) column chromatography (3 to 100% ethyl acetate in hexanes as eluant) to provide the title compound, a compound of the present invention, as a yellow oil (118 mg).

¹H NMR (CDCl₃): δ 7.18 (m, 1H), 7.10 (m, 1H), 6.96 (m, 1H), 6.80-6.65 (m, 3H), 3.83 (s, 2H), 3.70 (s, 3H), 2.11 (s, 3H).

### Example 42

### Preparation of 4-chloro-1-(4-chlorophenyl)-5-(2,4-difluorophenyl)-1H-1,2,3-triazole (Compound 24)

### Step A: Preparation of 1-azido-4-chlorobenzene

A stirred mixture of p-chloroaniline (1.0 g, 7.8 mmol) in trifluoroacetic acid (20 mL) was cooled to 0° C., and then a solution of sodium nitrite (2.7 g, 39.2 mmol) in water (10 mL) was added over 10 minutes. While maintaining the temperature of the reaction mixture at 0° C., a solution of sodium azide (5.1 g, 78.4 mmol) in water (10 mL) was added t over 10 minutes. The reaction mixture was allowed to warm to room temperature and stirred overnight. The reaction mixture was extracted with dichloromethane (2×), and the combined organic layers were washed with saturated aqueous sodium bicarbonate, dried over magnesium sulfate, filtered and concentrated under reduced pressure to provide the title compound as a brown oil (1.13 g).

¹H NMR (CDCl₃): δ 7.31 (m, 2H), 6.96 (m, 2H).

### Step B Preparation of 4-chloro-1-(4-chlorophenyl)-5-(2,4-difluorophenyl)-1H-1,2,3-triazole

To a mixture of ethylmagnesium chloride (2 M in tetrahydrofuran, 1.2 mL, 2.39 mmol) was added a solution of 1-ethynyl-2,4-difluorobenzene (0.300 g, 2.17 mmol) in tetrahydrofuran (1 mL). The reaction mixture was heated at 50° C. for 15 minutes and then allowed to cool to room temperature. A solution of 1-azido-4-chlorobenzene (i.e. the product of Step A) (0.328 g, 2.39 mmol) in tetrahydrofuran (1 mL) was added to the reaction mixture, followed by heating at 50° C. After 1 h, hexachloroethane (1.03 g, 4.34 mmol) was added to the reaction mixture. After 2 h, the reaction mixture was allowed to cool to room temperature, and hydrochloric acid (2 M in diethyl ether, 2 mL) was added. The reaction mixture was concentrated under reduced pressure, and the resulting material was purified by medium pressure liquid chromatography on silica gel (0 to 100% gradient of ethyl acetate in hexanes as eluant) to provide the title compound, a compound of the present invention, as a solid (0.35 g).

¹H NMR (CDCl₃): δ 7.40 (m, 2H), 7.36 (m, 1H), 7.26 (m, 2H), 7.03 (m, 1H), 6.89 (m, 1H).

### Example 43

### Preparation of 4-[4-chloro-5-(2,6-difluoro-4-methoxyphenyl)-2-methyl-1H-1-yl]phenyl thiocyanate (Compound 741)

A mixture of 4-chloro-1-(4-iodophenyl)-5-(2,6-difluoro-4-methoxyphenyl)-2-methyl-1H-imidazole (prepared by a procedure analogous to Example 13) (0.2 g, 0.43 mmol), cuprous thiocyanate (0.08 g, 0.65 mmol) and potassium thiocyanate (0.06 g, 0.65 mmol) in N,N-dimethylformamide (5 mL) was heated to 140° C. overnight. The reaction mixture was cooled to room temperature and then partitioned between water and ethyl acetate, the layers were separated, and the aqueous layer was extracted with ethyl acetate. The combined organic layers were dried over magnesium sulfate, filtered and concentrated under reduced pressure. The resulting material was purified by medium pressure liquid chromatography on silica gel (20:80 to 60:40 to 80:20 gradient of ethyl acetate in hexane as eluant) to provide the title compound, a compound of the present invention, as solid (0.07 g).

¹H NMR (CDCl₃): δ 7.53 (d, 2H), 7.22 (d, 2H), 6.40 (d, 2H), 3.78 (s, 3H), 2.31 (s, 3H).

By the procedures described herein together with methods known in the art, the following compounds of Tables 1 to 12 can be prepared. The following abbreviations are used in the Tables which follow: Me means methyl, Et means ethyl, Ph means phenyl, MeO means methoxy, MeS is methylthio, CN means cyano, Bn means benzyl and NO₂ means nitro.

TABLE 1

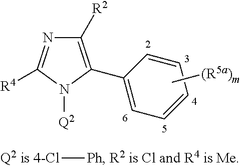

| (R^{5a})ₘ | (R^{5a})ₘ | (R^{5a})ₘ | (R^{5a})ₘ | (R^{5a})ₘ |
|---|---|---|---|---|
| 2,6-di-F | 2,4-di-Cl | 4-CN, 2,6-di-F | 2-CF₃, 4-F | 2-Cl, 4-NO₂ |
| 2,4,6-tri-F | 2-Cl, 4,6-di-F | 2,6-di-F, 4-Me | 2-CF₂HO, 4-F | 2-NO₂, 4-F |
| 2,3,6-tri-F | 2-Cl, 6-F | 2-Cl, 5-CF₃ | 2-CN, 6-F | 2,3,4,5,6-penta-F |
| 2,4,5-tri-F | 2-Br, 6-F | 2-Cl, 4-Me | 2,5-di-Cl | 2,5-di-Cl, 4-F |
| 2,3,4-tri-F | 2-F, 6-CF₃ | 2-Cl, 4-MeO | 2-CF₃, 4-MeO | 2,3-di-Cl, 4-F |
| 2-Cl, 4-F | 2-F, 6-CHF₂O | 2-Br, 4-MeO | 2-F, 6-Me | 2-Cl, 5-CN |
| 2-Br, 4-F | 2-I, 4-F | 2,6-di-F, 3-Cl | 2,6-di-F, 3-Me | 2,4-di-F, 5-CN |
| 2,4-di-F | 4-Cl, 2,6-di-F | 2,6-di-F, 3-CN | 2-CF₃ | 2-Cl, 6-F, 3-MeO |
| 2,6-di-Cl | 2,6-di-F, 4-MeO | 2,6-di-F, 3-MeO | 2-CF₃O | |
| 2,6-di-F, 4-EtO | 2-Cl, 6-F, 4-MeO | 2,6-di-F, 4-CF₂HO | 2,6-di-F, 4-NO₂ | |
| 2-Cl, 3,6-di-F | 2-Cl, 6-F, 5-MeO | 2,6-di-F, 3-CF₂HO | 2,6-di-F, 3-EtO | |

The present disclosure also includes Tables 1A through 934A, each of which is constructed the same as Table 1 above except that the row heading in Table 1 (i.e. “Q² is 4-Cl-Ph, R² is Cl and R⁴ is Me”) is replaced with the respective row heading shown below. For example, in Table 1A the row heading is “Q² is 4-Cl-Ph, R² is Br and R⁴ is Me”, and (R^{5a})ₘ is as defined in Table 1 above. Thus, the first entry in Table 1A specifically discloses 4-bromo-1-(4-chlorophenyl)-5-(2,6-difluorophenyl)-1H-imidazole. Tables 2A through 934A are constructed similarly.

| Table | Row Heading |
|---|---|
| 1A | Q² is 4-Cl-Ph, R² is Br and R⁴ is Me. |
| 2A | Q² is 4-Cl-Ph, R² is Cl and R⁴ is CFH₂. |
| 3A | Q² is 4-Cl-Ph, R² is I and R⁴ is Me. |
| 4A | Q² is 4-Cl-Ph, R² is Me and R⁴ is Me. |
| 5A | Q² is 4-Cl-Ph, R² is Me and R⁴ is Cl. |
| 6A | Q² is 4-Cl-Ph, R² is Me and R⁴ is Br. |
| 7A | Q² is 4-Cl-Ph, R² is Me and R⁴ is I. |
| 8A | Q² is 4-Cl-Ph, R² is Me and R⁴ is MeO. |
| 9A | Q² is 4-Cl-Ph, R² is MeO and R⁴ is Me. |
| 10A | Q² is 4-Cl-Ph, R² is Br and R⁴ is Br. |
| 11A | Q² is 4-Cl-Ph, R² is Br and R⁴ is Cl. |
| 12A | Q² is 4-Cl-Ph, R² is Cl and R⁴ is Br. |
| 13A | Q² is 4-Cl-Ph, R² is Cl and R⁴ is Cl. |
| 14A | Q² is 4-Cl-Ph, R² is Me and R⁴ is MeS. |
| 15A | Q² is 4-Cl-Ph, R² is MeS and R⁴ is Me. |
| 16A | Q² is 4-Cl-Ph, R² is Et and R⁴ is Br. |
| 17A | Q² is 4-Cl-Ph, R² is Et and R⁴ is Cl. |
| 18A | Q² is 4-Cl-Ph, R² is Et and R⁴ is Me. |
| 19A | Q² is 4-Cl-Ph, R² is Me and R⁴ is Et. |
| 20A | Q² is 4-Cl-Ph, R² is Cl and R⁴ is Et. |
| 21A | Q² is 4-Cl-Ph, R² is Me and R⁴ is CN. |
| 22A | Q² is 4-Cl-Ph, R² is Me and R⁴ is H. |
| 23A | Q² is 4-Cl-Ph, R² is Cl and R⁴ is H. |
| 24A | Q² is 4-Cl-Ph, R² is Br and R⁴ is H. |
| 25A | Q² is 3-Cl-Ph, R² is Cl and R⁴ is Me. |
| 26A | Q² is 3-Cl-Ph, R² is Cl and R⁴ is CFH₂. |
| 27A | Q² is 3-Cl-Ph, R² is Br and R⁴ is Me. |
| 28A | Q² is 3-Cl-Ph, R² is I and R⁴ is Me. |
| 29A | Q² is 3-Cl-Ph, R² is Me and R⁴ is Me. |
| 30A | Q² is 3-Cl-Ph, R² is Me and R⁴ is Cl. |
| 31A | Q² is 3-Cl-Ph, R² is Me and R⁴ is Br. |
| 32A | Q² is 3-Cl-Ph, R² is Me and R⁴ is I. |
| 33A | Q² is 3-Cl-Ph, R² is Br and R⁴ is Br. |
| 34A | Q² is 3-Cl-Ph, R² is Br and R⁴ is Cl. |
| 35A | Q² is 3-Cl-Ph, R² is Cl and R⁴ is Br. |
| 36A | Q² is 3-Cl-Ph, R² is Cl and R⁴ is Cl. |
| 37A | Q² is 3-Cl-Ph, R² is Me and R⁴ is H. |
| 38A | Q² is 3-Cl-Ph, R² is Cl and R⁴ is H. |
| 39A | Q² is 3-Cl-Ph, R² is Br and R⁴ is H |
| 40A | Q² is 4-F-Ph, R² is Cl and R⁴ is Me. |
| 41A | Q² is 4-F-Ph, R² is Cl and R⁴ is CFH₂. |
| 42A | Q² is 4-F-Ph, R² is Br and R⁴ is Me. |
| 43A | Q² is 4-F-Ph, R² is I and R⁴ is Me. |
| 44A | Q² is 4-F-Ph, R² is Me and R⁴ is Me. |
| 45A | Q² is 4-F-Ph, R² is Me and R⁴ is Cl. |
| 46A | Q² is 4-F-Ph, R² is Me and R⁴ is Br. |
| 47A | Q² is 4-F-Ph, R² is Me and R⁴ is I. |
| 48A | Q² is 4-F-Ph, R² is Br and R⁴ is Br. |
| 49A | Q² is 4-F-Ph, R² is Br and R⁴ is Cl. |
| 50A | Q² is 4-F-Ph, R² is Cl and R⁴ is Br. |
| 51A | Q² is 4-F-Ph, R² is Cl and R⁴ is Cl. |
| 52A | Q² is 4-F-Ph, R² is Me and R⁴ is H. |
| 53A | Q² is 4-F-Ph, R² is Cl and R⁴ is H. |
| 54A | Q² is 4-F-Ph, R² is Br and R⁴ is H. |
| 55A | Q² is 3-F-Ph, R² is Cl and R⁴ is Me. |
| 56A | Q² is 3-F-Ph, R² is Cl and R⁴ is CFH₂. |
| 57A | Q² is 3-F-Ph, R² is Br and R⁴ is Me. |
| 58A | Q² is 3-F-Ph, R² is I and R⁴ is Me. |
| 59A | Q² is 3-F-Ph, R² is Me and R⁴ is Me. |
| 60A | Q² is 3-F-Ph, R² is Me and R⁴ is Cl. |
| 61A | Q² is 3-F-Ph, R² is Me and R⁴ is Br. |
| 62A | Q² is 3-F-Ph, R² is Me and R⁴ is I. |
| 63A | Q² is 3-F-Ph, R² is Br and R⁴ is Br. |
| 64A | Q² is 3-F-Ph, R² is Br and R⁴ is Cl. |
| 65A | Q² is 3-F-Ph, R² is Cl and R⁴ is Br. |
| 66A | Q² is 3-F-Ph, R² is Cl and R⁴ is Cl. |
| 67A | Q² is 3-F-Ph, R² is Me and R⁴ is H. |
| 68A | Q² is 3-F-Ph, R² is Cl and R⁴ is H. |
| 69A | Q² is 3-F-Ph, R² is Br and R⁴ is H |
| 70A | Q² is 3-CF₂HO-Ph, R² is Cl and R⁴ is Me. |
| 71A | Q² is 3-CF₂HO-Ph, R² is Cl and R⁴ is CFH₂. |
| 72A | Q² is 3-CF₂HO-Ph, R² is Br and R⁴ is Me. |
| 73A | Q² is 3-CF₂HO-Ph, R² is I and R⁴ is Me. |
| 74A | Q² is 3-CF₂HO-Ph, R² is Me and R⁴ is Me. |
| 75A | Q² is 3-CF₂HO-Ph, R² is Me and R⁴ is Cl. |
| 76A | Q² is 3-CF₂HO-Ph, R² is Me and R⁴ is Br. |
| 77A | Q² is 3-CF₂HO-Ph, R² is Me and R⁴ is I. |
| 78A | Q² is 3-CF₂HO-Ph, R² is Br and R⁴ is Br. |
| 79A | Q² is 3-CF₂HO-Ph, R² is Br and R⁴ is Cl. |
| 80A | Q² is 3-CF₂HO-Ph, R² is Cl and R⁴ is Br. |
| 81A | Q² is 3-CF₂HO-Ph, R² is Cl and R⁴ is Cl. |
| 82A | Q² is 3-CF₂HO-Ph, R² is Me and R⁴ is H. |
| 83A | Q² is 3-CF₂HO-Ph, R² is Cl and R⁴ is H. |
| 84A | Q² is 3-CF₂HO-Ph, R² is Br and R⁴ is H |
| 85A | Q² is 4-Me-Ph, R² is Cl and R⁴ is Me. |
| 86A | Q² is 4-Me-Ph, R² is Cl and R⁴ is CFH₂. |
| 87A | Q² is 4-Me-Ph, R² is Br and R⁴ is Me. |
| 88A | Q² is 4-Me-Ph, R² is I and R⁴ is Me. |
| 89A | Q² is 4-Me-Ph, R² is Me and R⁴ is Me. |
| 90A | Q² is 4-Me-Ph, R² is Me and R⁴ is Cl. |
| 91A | Q² is 4-Me-Ph, R² is Me and R⁴ is Br. |
| 92A | Q² is 4-Me-Ph, R² is Me and R⁴ is I. |
| 93A | Q² is 4-Me-Ph, R² is Br and R⁴ is Br. |
| 94A | Q² is 4-Me-Ph, R² is Br and R⁴ is Cl. |
| 95A | Q² is 4-Me-Ph, R² is Cl and R⁴ is Br. |
| 96A | Q² is 4-Me-Ph, R² is Cl and R⁴ is Cl. |
| 97A | Q² is 4-Me-Ph, R² is Me and R⁴ is H. |
| 98A | Q² is 4-Me-Ph, R² is Cl and R⁴ is H. |
| 99A | Q² is 4-Me-Ph, R² is Br and R⁴ is H. |
| 100A | Q² is 3-Me-Ph, R² is Cl and R⁴ is Me. |
| 101A | Q² is 3-Me-Ph, R² is Cl and R⁴ is CFH₂. |
| 102A | Q² is 3-Me-Ph, R² is Br and R⁴ is Me. |
| 103A | Q² is 3-Me-Ph, R² is I and R⁴ is Me. |
| 104A | Q² is 3-Me-Ph, R² is Me and R⁴ is Me. |
| 105A | Q² is 3-Me-Ph, R² is Me and R⁴ is Cl. |
| 106A | Q² is 3-Me-Ph, R² is Me and R⁴ is Br. |
| 107A | Q² is 3-Me-Ph, R² is Me and R⁴ is I. |
| 108A | Q² is 3-Me-Ph, R² is Br and R⁴ is Br. |
| 109A | Q² is 3-Me-Ph, R² is Br and R⁴ is Cl. |
| 110A | Q² is 3-Me-Ph, R² is Cl and R⁴ is Br. |
| 111A | Q² is 3-Me-Ph, R² is Cl and R⁴ is Cl. |
| 112A | Q² is 3-Me-Ph, R² is Me and R⁴ is H. |
| 113A | Q² is 3-Me-Ph, R² is Cl and R⁴ is H. |
| 114A | Q² is 3-Me-Ph, R² is Br and R⁴ is H. |
| 115A | Q² is 4-Et-Ph, R² is Cl and R⁴ is Me. |
| 116A | Q² is 4-Et-Ph, R² is Cl and R⁴ is CFH₂. |
| 117A | Q² is 4-Et-Ph, R² is Br and R⁴ is Me. |
| 118A | Q² is 4-Et-Ph, R² is I and R⁴ is Me. |
| 119A | Q² is 4-Et-Ph, R² is Me and R⁴ is Me. |
| 120A | Q² is 4-Et-Ph, R² is Me and R⁴ is Cl. |
| 121A | Q² is 4-Et-Ph, R² is Me and R⁴ is Br. |
| 122A | Q² is 4-Et-Ph, R² is Me and R⁴ is I. |
| 123A | Q² is 4-Et-Ph, R² is Br and R⁴ is Br. |
| 124A | Q² is 4-Et-Ph, R² is Br and R⁴ is Cl. |
| 125A | Q² is 4-Et-Ph, R² is Cl and R⁴ is Br. |
| 126A | Q² is 4-Et-Ph, R² is Cl and R⁴ is Cl. |
| 127A | Q² is 4-Et-Ph, R² is Me and R⁴ is H. |
| 128A | Q² is 4-Et-Ph, R² is Cl and R⁴ is H. |
| 129A | Q² is 4-Et-Ph, R² is Br and R⁴ is H. |
| 130A | Q² is 4-Cl, 3-F-Ph, R² is Cl and R⁴ is Me. |
| 131A | Q² is 4-Cl, 3-F-Ph, R² is Cl and R⁴ is CFH₂. |
| 132A | Q² is 4-Cl, 3-F-Ph, R² is Br and R⁴ is Me. |
| 133A | Q² is 4-Cl, 3-F-Ph, R² is I and R⁴ is Me. |
| 134A | Q² is 4-Cl, 3-F-Ph, R² is Me and R⁴ is Me. |
| 135A | Q² is 4-Cl, 3-F-Ph, R² is Me and R⁴ is Cl. |
| 136A | Q² is 4-Cl, 3-F-Ph, R² is Me and R⁴ is Br. |
| 137A | Q² is 4-Cl, 3-F-Ph, R² is Me and R⁴ is I. |
| 138A | Q² is 4-Cl, 3-F-Ph, R² is Br and R⁴ is Br. |
| 139A | Q² is 4-Cl, 3-F-Ph, R² is Br and R⁴ is Cl. |
| 140A | Q² is 4-Cl, 3-F-Ph, R² is Cl and R⁴ is Br. |
| 141A | Q² is 4-Cl, 3-F-Ph, R² is Cl and R⁴ is Cl. |
| 142A | Q² is 4-Cl, 3-F-Ph, R² is Me and R⁴ is H. |
| 143A | Q² is 4-Cl, 3-F-Ph, R² is Cl and R⁴ is H. |
| 144A | Q² is 4-Cl, 3-F-Ph, R² is Br and R⁴ is H. |
| 145A | Q² is 2-Cl, 4-F-Ph, R² is Cl and R⁴ is Me. |
| 146A | Q² is 2-Cl, 4-F-Ph, R² is Cl and R⁴ is CFH₂. |
| 147A | Q² is 2-Cl, 4-F-Ph, R² is Br and R⁴ is Me. |
| 148A | Q² is 2-Cl, 4-F-Ph, R² is I and R⁴ is Me. |
| 149A | Q² is 2-Cl, 4-F-Ph, R² is Me and R⁴ is Me. |
| 150A | Q² is 2-Cl, 4-F-Ph, R² is Me and R⁴ is Cl. |
| 151A | Q² is 2-Cl, 4-F-Ph, R² is Me and R⁴ is Br. |
| 152A | Q² is 2-Cl, 4-F-Ph, R² is Me and R⁴ is I. |
| 153A | Q² is 2-Cl, 4-F-Ph, R² is Br and R⁴ is Br. |
| 154A | Q² is 2-Cl, 4-F-Ph, R² is Br and R⁴ is Cl. |
| 155A | Q² is 2-Cl, 4-F-Ph, R² is Cl and R⁴ is Br. |
| 156A | Q² is 2-Cl, 4-F-Ph, R² is Cl and R⁴ is Cl. |
| 157A | Q² is 2-Cl, 4-F-Ph, R² is Me and R⁴ is H. |
| 158A | Q² is 2-Cl, 4-F-Ph, R² is Cl and R⁴ is H. |
| 159A | Q² is 2-Cl, 4-F-Ph, R² is Br and R⁴ is H. |
| 160A | Q² is 4-F, 3-Me-Ph, R² is Cl and R⁴ is Me. |
| 161A | Q² is 4-F, 3-Me-Ph, R² is Cl and R⁴ is CFH₂. |
| 162A | Q² is 4-F, 3-Me-Ph, R² is Br and R⁴ is Me. |
| 163A | Q² is 4-F, 3-Me-Ph, R² is I and R⁴ is Me. |
| 164A | Q² is 4-F, 3-Me-Ph, R² is Me and R⁴ is Me. |
| 165A | Q² is 4-F, 3-Me-Ph, R² is Me and R⁴ is Cl. |
| 166A | Q² is 4-F, 3-Me-Ph, R² is Me and R⁴ is Br. |
| 167A | Q² is 4-F, 3-Me-Ph, R² is Me and R⁴ is I. |
| 168A | Q² is 4-F, 3-Me-Ph, R² is Br and R⁴ is Br. |
| 169A | Q² is 4-F, 3-Me-Ph, R² is Br and R⁴ is Cl. |
| 170A | Q² is 4-F, 3-Me-Ph, R² is Cl and R⁴ is Br. |
| 171A | Q² is 4-F, 3-Me-Ph, R² is Cl and R⁴ is Cl. |
| 172A | Q² is 4-F, 3-Me-Ph, R² is Me and R⁴ is H. |
| 173A | Q² is 4-F, 3-Me-Ph, R² is Cl and R⁴ is H. |
| 174A | Q² is 4-F, 3-Me-Ph, R² is Br and R⁴ is H. |
| 175A | Q² is 3,4-di-F-Ph, R² is Cl and R⁴ is Me. |
| 176A | Q² is 3,4-di-F-Ph, R² is Cl and R⁴ is CFH₂. |
| 177A | Q² is 3,4-di-F-Ph, R² is Br and R⁴ is Me. |
| 178A | Q² is 3,4-di-F-Ph, R² is I and R⁴ is Me. |
| 179A | Q² is 3,4-di-F-Ph, R² is Me and R⁴ is Me. |
| 180A | Q² is 3,4-di-F-Ph, R² is Me and R⁴ is Cl. |
| 181A | Q² is 3,4-di-F-Ph, R² is Me and R⁴ is Br. |
| 182A | Q² is 3,4-di-F-Ph, R² is Me and R⁴ is I. |
| 183A | Q² is 3,4-di-F-Ph, R² is Br and R⁴ is Br. |
| 184A | Q² is 3,4-di-F-Ph, R² is Br and R⁴ is Cl. |
| 185A | Q² is 3,4-di-F-Ph, R² is Cl and R⁴ is Br. |
| 186A | Q² is 3,4-di-F-Ph, R² is Cl and R⁴ is Cl. |
| 187A | Q² is 3,4-di-F-Ph, R² is Me and R⁴ is H. |
| 188A | Q² is 3,4-di-F-Ph, R² is Cl and R⁴ is H. |
| 189A | Q² is 3,4-di-F-Ph, R² is Br and R⁴ is H. |
| 190A | Q² is 3,4-di-Cl-Ph, R² is Cl and R⁴ is Me. |
| 191A | Q² is 3,4-di-Cl-Ph, R² is Cl and R⁴ is CFH₂. |
| 192A | Q² is 3,4-di-Cl-Ph, R² is Br and R⁴ is Me. |
| 193A | Q² is 3,4-di-Cl-Ph, R² is I and R⁴ is Me. |
| 194A | Q² is 3,4-di-Cl-Ph, R² is Me and R⁴ is Me. |
| 195A | Q² is 3,4-di-Cl-Ph, R² is Me and R⁴ is Cl. |
| 196A | Q² is 3,4-di-Cl-Ph, R² is Me and R⁴ is Br. |
| 197A | Q² is 3,4-di-Cl-Ph, R² is Me and R⁴ is I. |
| 198A | Q² is 3,4-di-Cl-Ph, R² is Br and R⁴ is Br. |
| 199A | Q² is 3,4-di-Cl-Ph, R² is Br and R⁴ is Cl. |
| 200A | Q² is 3,4-di-Cl-Ph, R² is Cl and R⁴ is Br. |
| 201A | Q² is 3,4-di-Cl-Ph, R² is Cl and R⁴ is Cl. |
| 202A | Q² is 3,4-di-Cl-Ph, R² is Me and R⁴ is H. |
| 203A | Q² is 3,4-di-Cl-Ph, R² is Cl and R⁴ is H. |
| 204A | Q² is 3,4-di-Cl-Ph, R² is Br and R⁴ is H. |
| 205A | Q² is 3,5-di-MeO-Ph, R² is Cl and R⁴ is Me. |
| 206A | Q² is 3,5-di-MeO-Ph, R² is Cl and R⁴ is CFH₂. |
| 207A | Q² is 3,5-di-MeO-Ph, R² is Br and R⁴ is Me. |
| 208A | Q² is 3,5-di-MeO-Ph, R² is I and R⁴ is Me. |
| 209A | Q² is 3,5-di-MeO-Ph, R² is Me and R⁴ is Me. |
| 210A | Q² is 3,5-di-MeO-Ph, R² is Me and R⁴ is Cl. |
| 211A | Q² is 3,5-di-MeO-Ph, R² is Me and R⁴ is Br. |
| 212A | Q² is 3,5-di-MeO-Ph, R² is Me and R⁴ is I. |
| 213A | Q² is 3,5-di-MeO-Ph, R² is Br and R⁴ is Br. |
| 214A | Q² is 3,5-di-MeO-Ph, R² is Br and R⁴ is Cl. |
| 215A | Q² is 3,5-di-MeO-Ph, R² is Cl and R⁴ is Br. |
| 216A | Q² is 3,5-di-MeO-Ph, R² is Cl and R⁴ is Cl. |
| 217A | Q² is 3,5-di-MeO-Ph, R² is Me and R⁴ is H. |
| 218A | Q² is 3,5-di-MeO-Ph, R² is Cl and R⁴ is H. |
| 219A | Q² is 3,5-di-MeO-Ph, R² is Br and R⁴ is H. |
| 220A | Q² is 2-Cl, 3,5-di-MeO-Ph, R² is Cl and R⁴ is Me. |
| 221A | Q² is 2-Cl, 3,5-di-MeO-Ph, R² is Cl and R⁴ is CFH₂. |
| 222A | Q² is 2-Cl, 3,5-di-MeO-Ph, R² is Br and R⁴ is Me. |
| 223A | Q² is 2-Cl, 3,5-di-MeO-Ph, R² is I and R⁴ is Me. |
| 224A | Q² is 2-Cl, 3,5-di-MeO-Ph, R² is Me and R⁴ is Me. |
| 225A | Q² is 2-Cl, 3,5-di-MeO-Ph, R² is Me and R⁴ is Cl. |
| 226A | Q² is 2-Cl, 3,5-di-MeO-Ph, R² is Me and R⁴ is Br. |
| 227A | Q² is 2-Cl, 3,5-di-MeO-Ph, R² is Me and R⁴ is I. |
| 228A | Q² is 2-Cl, 3,5-di-MeO-Ph, R² is Br and R⁴ is Br. |
| 229A | Q² is 2-Cl, 3,5-di-MeO-Ph, R² is Br and R⁴ is Cl. |
| 230A | Q² is 2-Cl, 3,5-di-MeO-Ph, R² is Cl and R⁴ is Br. |
| 231A | Q² is 2-Cl, 3,5-di-MeO-Ph, R² is Cl and R⁴ is Cl. |
| 232A | Q² is 2-Cl, 3,5-di-MeO-Ph, R² is Me and R⁴ is H. |
| 233A | Q² is 2-Cl, 3,5-di-MeO-Ph, R² is Cl and R⁴ is H. |
| 234A | Q² is 2-Cl, 3,5-di-MeO-Ph, R² is Br and R⁴ is H. |
| 235A | Q² is 4-Cl, 3,5-di-MeO-Ph, R² is Cl and R⁴ is Me. |
| 236A | Q² is 4-Cl, 3,5-di-MeO-Ph, R² is Cl and R⁴ is CFH₂. |
| 237A | Q² is 4-Cl, 3,5-di-MeO-Ph, R² is Br and R⁴ is Me. |
| 238A | Q² is 4-Cl, 3,5-di-MeO-Ph, R² is I and R⁴ is Me. |
| 239A | Q² is 4-Cl, 3,5-di-MeO-Ph, R² is Me and R⁴ is Me. |
| 240A | Q² is 4-Cl, 3,5-di-MeO-Ph, R² is Me and R⁴ is Cl. |
| 241A | Q² is 4-Cl, 3,5-di-MeO-Ph, R² is Me and R⁴ is Br. |
| 242A | Q² is 4-Cl, 3,5-di-MeO-Ph, R² is Me and R⁴ is I. |
| 243A | Q² is 4-Cl, 3,5-di-MeO-Ph, R² is Br and R⁴ is Br. |
| 244A | Q² is 4-Cl, 3,5-di-MeO-Ph, R² is Br and R⁴ is Cl. |
| 245A | Q² is 4-Cl, 3,5-di-MeO-Ph, R² is Cl and R⁴ is Br. |
| 246A | Q² is 4-Cl, 3,5-di-MeO-Ph, R² is Cl and R⁴ is Cl. |
| 247A | Q² is 4-Cl, 3,5-di-MeO-Ph, R² is Me and R⁴ is H. |
| 248A | Q² is 4-Cl, 3,5-di-MeO-Ph, R² is Cl and R⁴ is H. |
| 249A | Q² is 4-Cl, 3,5-di-MeO-Ph, R² is Br and R⁴ is H. |
| 250A | Q² is 4-Cl-Bn, R² is Cl and R⁴ is Me. |
| 251A | Q² is 4-Cl-Bn, R² is Cl and R⁴ is CFH₂. |
| 252A | Q² is 4-Cl-Bn, R² is Br and R⁴ is Me. |
| 253A | Q² is 4-Cl-Bn, R² is I and R⁴ is Me. |
| 254A | Q² is 4-Cl-Bn, R² is Me and R⁴ is Me. |
| 255A | Q² is 4-Cl-Bn, R² is Me and R⁴ is Cl. |
| 256A | Q² is 4-Cl-Bn, R² is Me and R⁴ is Br. |
| 257A | Q² is 4-Cl-Bn, R² is Me and R⁴ is I. |
| 258A | Q² is 4-Cl-Bn, R² is Br and R⁴ is Br. |
| 259A | Q² is 4-Cl-Bn, R² is Br and R⁴ is Cl. |
| 260A | Q² is 4-Cl-Bn, R² is Cl and R⁴ is Br. |
| 261A | Q² is 4-Cl-Bn, R² is Cl and R⁴ is Cl. |
| 262A | Q² is 4-Cl-Bn, R² is Me and R⁴ is H. |
| 263A | Q² is 4-Cl-Bn, R² is Cl and R⁴ is H. |
| 264A | Q² is 4-Cl-Bn, R² is Br and R⁴ is H. |
| 265A | Q² is 4-F-Bn, R² is Cl and R⁴ is Me. |
| 266A | Q² is 4-F-Bn, R² is Cl and R⁴ is CFH₂. |
| 267A | Q² is 4-F-Bn, R² is Br and R⁴ is Me. |
| 268A | Q² is 4-F-Bn, R² is I and R⁴ is Me. |
| 269A | Q² is 4-F-Bn, R² is Me and R⁴ is Me. |
| 270A | Q² is 4-F-Bn, R² is Me and R⁴ is Cl. |
| 271A | Q² is 4-F-Bn, R² is Me and R⁴ is Br. |
| 272A | Q² is 4-F-Bn, R² is Me and R⁴ is I. |
| 273A | Q² is 4-F-Bn, R² is Br and R⁴ is Br. |
| 274A | Q² is 4-F-Bn, R² is Br and R⁴ is Cl. |
| 275A | Q² is 4-F-Bn, R² is Cl and R⁴ is Br. |
| 276A | Q² is 4-F-Bn, R² is Cl and R⁴ is Cl. |
| 277A | Q² is 4-F-Bn, R² is Me and R⁴ is H. |
| 278A | Q² is 4-F-Bn, R² is Cl and R⁴ is H. |
| 279A | Q² is 4-F-Bn, R² is Br and R⁴ is H. |
| 280A | Q² is 6-Cl-3-pyridinyl, R² is Cl and R⁴ is Me. |
| 281A | Q² is 6-Cl-3-pyridinyl, R² is Cl and R⁴ is CFH₂. |
| 282A | Q² is 6-Cl-3-pyridinyl, R² is Br and R⁴ is Me. |
| 283A | Q² is 6-Cl-3-pyridinyl, R² is I and R⁴ is Me. |
| 284A | Q² is 6-Cl-3-pyridinyl, R² is Me and R⁴ is Me. |
| 285A | Q² is 6-Cl-3-pyridinyl, R² is Me and R⁴ is Cl. |
| 286A | Q² is 6-Cl-3-pyridinyl, R² is Me and R⁴ is Br. |
| 287A | Q² is 6-Cl-3-pyridinyl, R² is Me and R⁴ is I. |
| 288A | Q² is 6-Cl-3-pyridinyl, R² is Me and R⁴ is MeO. |
| 289A | Q² is 6-Cl-3-pyridinyl, R² is MeO and R⁴ is Me. |
| 290A | Q² is 6-Cl-3-pyridinyl, R² is Br and R⁴ is Br. |
| 291A | Q² is 6-Cl-3-pyridinyl, R² is Br and R⁴ is Cl. |
| 292A | Q² is 6-Cl-3-pyridinyl, R² is Cl and R⁴ is Br. |
| 293A | Q² is 6-Cl-3-pyridinyl, R² is Cl and R⁴ is Cl. |
| 294A | Q² is 6-Cl-3-pyridinyl, R² is Me and R⁴ is MeS. |
| 295A | Q² is 6-Cl-3-pyridinyl, R² is MeS and R⁴ is Me. |
| 296A | Q² is 6-Cl-3-pyridinyl, R² is Et and R⁴ is Br. |
| 297A | Q² is 6-Cl-3-pyridinyl, R² is Et and R⁴ is Cl. |
| 298A | Q² is 6-Cl-3-pyridinyl, R² is Et and R⁴ is Me. |
| 299A | Q² is 6-Cl-3-pyridinyl, R² is Me and R⁴ is Et. |
| 300A | Q² is 6-Cl-3-pyridinyl, R² is Cl and R⁴ is Et. |
| 301A | Q² is 6-Cl-3-pyridinyl, R² is Me and R⁴ is CN. |
| 302A | Q² is 6-Cl-3-pyridinyl, R² is Me and R⁴ is H. |
| 303A | Q² is 6-Cl-3-pyridinyl, R² is Cl and R⁴ is H. |
| 304A | Q² is 6-Cl-3-pyridinyl, R² is Br and R⁴ is H. |
| 305A | Q² is 6-Me-3-pyridinyl, R² is Cl and R⁴ is Me. |
| 306A | Q² is 6-Me-3-pyridinyl, R² is Cl and R⁴ is CFH₂. |
| 307A | Q² is 6-Me-3-pyridinyl, R² is Br and R⁴ is Me. |
| 308A | Q² is 6-Me-3-pyridinyl, R² is I and R⁴ is Me. |
| 309A | Q² is 6-Me-3-pyridinyl, R² is Me and R⁴ is Me. |
| 310A | Q² is 6-Me-3-pyridinyl, R² is Me and R⁴ is Cl. |
| 311A | Q² is 6-Me-3-pyridinyl, R² is Me and R⁴ is Br. |
| 312A | Q² is 6-Me-3-pyridinyl, R² is Me and R⁴ is I. |
| 313A | Q² is 6-Me-3-pyridinyl, R² is Br and R⁴ is Br. |
| 314A | Q² is 6-Me-3-pyridinyl, R² is Br and R⁴ is Cl. |
| 315A | Q² is 6-Me-3-pyridinyl, R² is Cl and R⁴ is Br. |
| 316A | Q² is 6-Me-3-pyridinyl, R² is Cl and R⁴ is Cl. |
| 317A | Q² is 6-Me-3-pyridinyl, R² is Me and R⁴ is H. |
| 318A | Q² is 6-Me-3-pyridinyl, R² is Cl and R⁴ is H. |
| 319A | Q² is 6-Me-3-pyridinyl, R² is Br and R⁴ is H. |
| 320A | Q² is 6-MeO-3-pyridinyl, R² is Cl and R⁴ is Me. |
| 321A | Q² is 6-MeO-3-pyridinyl, R² is Cl and R⁴ is CFH₂. |
| 322A | Q² is 6-MeO-3-pyridinyl, R² is Br and R⁴ is Me. |
| 323A | Q² is 6-MeO-3-pyridinyl, R² is I and R⁴ is Me. |
| 324A | Q² is 6-MeO-3-pyridinyl, R² is Me and R⁴ is Me. |
| 325A | Q² is 6-MeO-3-pyridinyl, R² is Me and R⁴ is Cl. |
| 326A | Q² is 6-MeO-3-pyridinyl, R² is Me and R⁴ is Br. |
| 327A | Q² is 6-MeO-3-pyridinyl, R² is Me and R⁴ is I. |
| 328A | Q² is 6-MeO-3-pyridinyl, R² is Br and R⁴ is Br. |
| 329A | Q² is 6-MeO-3-pyridinyl, R² is Br and R⁴ is Cl. |
| 330A | Q² is 6-MeO-3-pyridinyl, R² is Cl and R⁴ is Br. |
| 331A | Q² is 6-MeO-3-pyridinyl, R² is Cl and R⁴ is Cl. |
| 332A | Q² is 6-MeO-3-pyridinyl, R² is Me and R⁴ is H. |
| 333A | Q² is 6-MeO-3-pyridinyl, R² is Cl and R⁴ is H. |
| 334A | Q² is 6-MeO-3-pyridinyl, R² is Br and R⁴ is H. |
| 335A | Q² is 6-CF₃-3-pyridinyl, R² is Cl and R⁴ is Me. |
| 336A | Q² is 6-CF₃-3-pyridinyl, R² is Cl and R⁴ is CFH₂. |
| 337A | Q² is 6-CF₃-3-pyridinyl, R² is Br and R⁴ is Me. |
| 338A | Q² is 6-CF₃-3-pyridinyl, R² is I and R⁴ is Me. |
| 339A | Q² is 6-CF₃-3-pyridinyl, R² is Me and R⁴ is Me. |
| 340A | Q² is 6-CF₃-3-pyridinyl, R² is Me and R⁴ is Cl. |
| 341A | Q² is 6-CF₃-3-pyridinyl, R² is Me and R⁴ is Br. |
| 342A | Q² is 6-CF₃-3-pyridinyl, R² is Me and R⁴ is I. |
| 343A | Q² is 6-CF₃-3-pyridinyl, R² is Br and R⁴ is Br. |
| 344A | Q² is 6-CF₃-3-pyridinyl, R² is Br and R⁴ is Cl. |
| 345A | Q² is 6-CF₃-3-pyridinyl, R² is Cl and R⁴ is Br. |
| 346A | Q² is 6-CF₃-3-pyridinyl, R² is Cl and R⁴ is Cl. |
| 347A | Q² is 6-CF₃-3-pyridinyl, R² is Me and R⁴ is H. |
| 348A | Q² is 6-CF₃-3-pyridinyl, R² is Cl and R⁴ is H. |
| 349A | Q² is 6-CF₃-3-pyridinyl, R² is Br and R⁴ is H. |
| 350A | Q² is 6-Br-3-pyridinyl, R² is Cl and R⁴ is Me. |
| 351A | Q² is 6-Br-3-pyridinyl, R² is Cl and R⁴ is CFH₂. |
| 352A | Q² is 6-Br-3-pyridinyl, R² is Br and R⁴ is Me. |
| 353A | Q² is 6-Br-3-pyridinyl, R² is I and R⁴ is Me. |
| 354A | Q² is 6-Br-3-pyridinyl, R² is Me and R⁴ is Me. |
| 355A | Q² is 6-Br-3-pyridinyl, R² is Me and R⁴ is Cl. |
| 356A | Q² is 6-Br-3-pyridinyl, R² is Me and R⁴ is Br. |
| 357A | Q² is 6-Br-3-pyridinyl, R² is Me and R⁴ is I. |
| 358A | Q² is 6-Br-3-pyridinyl, R² is Br and R⁴ is Br. |
| 359A | Q² is 6-Br-3-pyridinyl, R² is Br and R⁴ is Cl. |
| 360A | Q² is 6-Br-3-pyridinyl, R² is Cl and R⁴ is Br. |
| 361A | Q² is 6-Br-3-pyridinyl, R² is Cl and R⁴ is Cl. |
| 362A | Q² is 6-Br-3-pyridinyl, R² is Me and R⁴ is H. |
| 363A | Q² is 6-Br-3-pyridinyl, R² is Cl and R⁴ is H. |
| 364A | Q² is 6-Br-3-pyridinyl, R² is Br and R⁴ is H. |
| 365A | Q² is 6-F-3-pyridinyl, R² is Cl and R⁴ is Me. |
| 366A | Q² is 6-F-3-pyridinyl, R² is Cl and R⁴ is CFH₂. |
| 367A | Q² is 6-F-3-pyridinyl, R² is Br and R⁴ is Me. |
| 368A | Q² is 6-F-3-pyridinyl, R² is I and R⁴ is Me. |
| 369A | Q² is 6-F-3-pyridinyl, R² is Me and R⁴ is Me. |
| 370A | Q² is 6-F-3-pyridinyl, R² is Me and R⁴ is Cl. |
| 371A | Q² is 6-F-3-pyridinyl, R² is Me and R⁴ is Br. |
| 372A | Q² is 6-F-3-pyridinyl, R² is Me and R⁴ is I. |
| 373A | Q² is 6-F-3-pyridinyl, R² is Br and R⁴ is Br. |
| 374A | Q² is 6-F-3-pyridinyl, R² is Br and R⁴ is Cl. |
| 375A | Q² is 6-F-3-pyridinyl, R² is Cl and R⁴ is Br. |
| 376A | Q² is 6-F-3-pyridinyl, R² is Cl and R⁴ is Cl. |
| 377A | Q² is 6-F-3-pyridinyl, R² is Me and R⁴ is H. |
| 378A | Q² is 6-F-3-pyridinyl, R² is Cl and R⁴ is H. |
| 379A | Q² is 6-F-3-pyridinyl, R² is Br and R⁴ is H. |
| 380A | Q² is 2-Cl, 6-Me-4-pyridinyl, R² is Cl and R⁴ is Me. |
| 381A | Q² is 2-Cl, 6-Me-4-pyridinyl, R² is Cl and R⁴ is CFH₂. |
| 382A | Q² is 2-Cl, 6-Me-4-pyridinyl, R² is Br and R⁴ is Me. |
| 383A | Q² is 2-Cl, 6-Me-4-pyridinyl, R² is I and R⁴ is Me. |
| 384A | Q² is 2-Cl, 6-Me-4-pyridinyl, R² is Me and R⁴ is Me. |
| 385A | Q² is 2-Cl, 6-Me-4-pyridinyl, R² is Me and R⁴ is Cl. |
| 386A | Q² is 2-Cl, 6-Me-4-pyridinyl, R² is Me and R⁴ is Br. |
| 387A | Q² is 2-Cl, 6-Me-4-pyridinyl, R² is Me and R⁴ is I. |
| 388A | Q² is 2-Cl, 6-Me-4-pyridinyl, R² is Br and R⁴ is Br. |
| 389A | Q² is 2-Cl, 6-Me-4-pyridinyl, R² is Br and R⁴ is Cl. |
| 390A | Q² is 2-Cl, 6-Me-4-pyridinyl, R² is Cl and R⁴ is Br. |
| 391A | Q² is 2-Cl, 6-Me-4-pyridinyl, R² is Cl and R⁴ is Cl. |
| 392A | Q² is 2-Cl, 6-Me-4-pyridinyl, R² is Me and R⁴ is H. |
| 393A | Q² is 2-Cl, 6-Me-4-pyridinyl, R² is Cl and R⁴ is H. |
| 394A | Q² is 2-Cl, 6-Me-4-pyridinyl, R² is Br and R⁴ is H. |
| 395A | Q² is 2-Cl, 6-MeO-3-pyridinyl, R² is Cl and R⁴ is Me. |
| 396A | Q² is 2-Cl, 6-MeO-3-pyridinyl, R² is Cl and R⁴ is CFH₂. |
| 397A | Q² is 2-Cl, 6-MeO-3-pyridinyl, R² is Br and R⁴ is Me. |
| 398A | Q² is 2-Cl, 6-MeO-3-pyridinyl, R² is I and R⁴ is Me. |
| 399A | Q² is 2-Cl, 6-MeO-3-pyridinyl, R² is Me and R⁴ is Me. |
| 400A | Q² is 2-Cl, 6-MeO-3-pyridinyl, R² is Me and R⁴ is Cl. |
| 401A | Q² is 2-Cl, 6-MeO-3-pyridinyl, R² is Me and R⁴ is Br. |
| 402A | Q² is 2-Cl, 6-MeO-3-pyridinyl, R² is Me and R⁴ is I. |
| 403A | Q² is 2-Cl, 6-MeO-3-pyridinyl, R² is Br and R⁴ is Br. |
| 404A | Q² is 2-Cl, 6-MeO-3-pyridinyl, R² is Br and R⁴ is Cl. |
| 405A | Q² is 2-Cl, 6-MeO-3-pyridinyl, R² is Cl and R⁴ is Br. |
| 406A | Q² is 2-Cl, 6-MeO-3-pyridinyl, R² is Cl and R⁴ is Cl. |
| 407A | Q² is 2-Cl, 6-MeO-3-pyridinyl, R² is Me and R⁴ is H. |
| 408A | Q² is 2-Cl, 6-MeO-3-pyridinyl, R² is Cl and R⁴ is H. |
| 409A | Q² is 2-Cl, 6-MeO-3-pyridinyl, R² is Br and R⁴ is H. |
| 410A | Q² is 2-Cl, 6-CF₃-3-pyridinyl, R² is Cl and R⁴ is Me. |
| 411A | Q² is 2-Cl, 6-CF₃-3-pyridinyl, R² is Cl and R⁴ is CFH₂. |
| 412A | Q² is 2-Cl, 6-CF₃-3-pyridinyl, R² is Br and R⁴ is Me. |
| 413A | Q² is 2-Cl, 6-CF₃-3-pyridinyl, R² is I and R⁴ is Me. |
| 414A | Q² is 2-Cl, 6-CF₃-3-pyridinyl, R² is Me and R⁴ is Me. |
| 415A | Q² is 2-Cl, 6-CF₃-3-pyridinyl, R² is Me and R⁴ is Cl. |
| 416A | Q² is 2-Cl, 6-CF₃-3-pyridinyl, R² is Me and R⁴ is Br. |
| 417A | Q² is 2-Cl, 6-CF₃-3-pyridinyl, R² is Me and R⁴ is I. |
| 418A | Q² is 2-Cl, 6-CF₃-3-pyridinyl, R² is Br and R⁴ is Br. |
| 419A | Q² is 2-Cl, 6-CF₃-3-pyridinyl, R² is Br and R⁴ is Cl. |
| 420A | Q² is 2-Cl, 6-CF₃-3-pyridinyl, R² is Cl and R⁴ is Br. |
| 421A | Q² is 2-Cl, 6-CF₃-3-pyridinyl, R² is Cl and R⁴ is Cl. |
| 422A | Q² is 2-Cl, 6-CF₃-3-pyridinyl, R² is Me and R⁴ is H. |
| 423A | Q² is 2-Cl, 6-CF₃-3-pyridinyl, R² is Cl and R⁴ is H. |
| 424A | Q² is 2-Cl, 6-CF₃-3-pyridinyl, R² is Br and R⁴ is H. |
| 425A | Q² is 5-Cl-3-pyridinyl, R² is Cl and R⁴ is Me. |
| 426A | Q² is 5-Cl-3-pyridinyl, R² is Cl and R⁴ is CFH₂. |
| 427A | Q² is 5-Cl-3-pyridinyl, R² is Br and R⁴ is Me. |
| 428A | Q² is 5-Cl-3-pyridinyl, R² is I and R⁴ is Me. |
| 429A | Q² is 5-Cl-3-pyridinyl, R² is Me and R⁴ is Me. |
| 430A | Q² is 5-Cl-3-pyridinyl, R² is Me and R⁴ is Cl. |
| 431A | Q² is 5-Cl-3-pyridinyl, R² is Me and R⁴ is Br. |
| 432A | Q² is 5-Cl-3-pyridinyl, R² is Me and R⁴ is I. |
| 433A | Q² is 5-Cl-3-pyridinyl, R² is Br and R⁴ is Br. |
| 434A | Q² is 5-Cl-3-pyridinyl, R² is Br and R⁴ is Cl. |
| 435A | Q² is 5-Cl-3-pyridinyl, R² is Cl and R⁴ is Br. |
| 436A | Q² is 5-Cl-3-pyridinyl, R² is Cl and R⁴ is Cl. |
| 437A | Q² is 5-Cl-3-pyridinyl, R² is Me and R⁴ is H. |
| 438A | Q² is 5-Cl-3-pyridinyl, R² is Cl and R⁴ is H. |
| 439A | Q² is 5-Cl-3-pyridinyl, R² is Br and R⁴ is H. |
| 440A | Q² is 5-F-3-pyridinyl, R² is Cl and R⁴ is Me. |
| 441A | Q² is 5-F-3-pyridinyl, R² is Cl and R⁴ is CFH₂. |
| 442A | Q² is 5-F-3-pyridinyl, R² is Br and R⁴ is Me. |
| 443A | Q² is 5-F-3-pyridinyl, R² is I and R⁴ is Me. |
| 444A | Q² is 5-F-3-pyridinyl, R² is Me and R⁴ is Me. |
| 445A | Q² is 5-F-3-pyridinyl, R² is Me and R⁴ is Cl. |
| 446A | Q² is 5-F-3-pyridinyl, R² is Me and R⁴ is Br. |
| 447A | Q² is 5-F-3-pyridinyl, R² is Me and R⁴ is I. |
| 448A | Q² is 5-F-3-pyridinyl, R² is Br and R⁴ is Br. |
| 449A | Q² is 5-F-3-pyridinyl, R² is Br and R⁴ is Cl. |
| 450A | Q² is 5-F-3-pyridinyl, R² is Cl and R⁴ is Br. |
| 451A | Q² is 5-F-3-pyridinyl, R² is Cl and R⁴ is Cl. |
| 452A | Q² is 5-F-3-pyridinyl, R² is Me and R⁴ is H. |
| 453A | Q² is 5-F-3-pyridinyl, R² is Cl and R⁴ is H. |
| 454A | Q² is 5-F-3-pyridinyl, R² is Br and R⁴ is H. |
| 455A | Q² is 5-Me-3-pyridinyl, R² is Cl and R⁴ is Me. |
| 456A | Q² is 5-Me-3-pyridinyl, R² is Cl and R⁴ is CFH₂. |
| 457A | Q² is 5-Me-3-pyridinyl, R² is Br and R⁴ is Me. |
| 458A | Q² is 5-Me-3-pyridinyl, R² is I and R⁴ is Me. |
| 459A | Q² is 5-Me-3-pyridinyl, R² is Me and R⁴ is Me. |
| 460A | Q² is 5-Me-3-pyridinyl, R² is Me and R⁴ is Cl. |
| 461A | Q² is 5-Me-3-pyridinyl, R² is Me and R⁴ is Br. |
| 462A | Q² is 5-Me-3-pyridinyl, R² is Me and R⁴ is I. |
| 463A | Q² is 5-Me-3-pyridinyl, R² is Br and R⁴ is Br. |
| 464A | Q² is 5-Me-3-pyridinyl, R² is Br and R⁴ is Cl. |
| 465A | Q² is 5-Me-3-pyridinyl, R² is Cl and R⁴ is Br. |
| 466A | Q² is 5-Me-3-pyridinyl, R² is Cl and R⁴ is Cl. |
| 467A | Q² is 5-Me-3-pyridinyl, R² is Me and R⁴ is H. |
| 468A | Q² is 5-Me-3-pyridinyl, R² is Cl and R⁴ is H. |
| 469A | Q² is 5-Me-3-pyridinyl, R² is Br and R⁴ is H. |
| 470A | Q² is 5-MeO-3-pyridinyl, R² is Cl and R⁴ is Me. |
| 471A | Q² is 5-MeO-3-pyridinyl, R² is Cl and R⁴ is CFH₂. |
| 472A | Q² is 5-MeO-3-pyridinyl, R² is Br and R⁴ is Me. |
| 473A | Q² is 5-MeO-3-pyridinyl, R² is I and R⁴ is Me. |
| 474A | Q² is 5-MeO-3-pyridinyl, R² is Me and R⁴ is Me. |
| 475A | Q² is 5-MeO-3-pyridinyl, R² is Me and R⁴ is Cl. |
| 476A | Q² is 5-MeO-3-pyridinyl, R² is Me and R⁴ is Br. |
| 477A | Q² is 5-MeO-3-pyridinyl, R² is Me and R⁴ is I. |
| 478A | Q² is 5-MeO-3-pyridinyl, R² is Br and R⁴ is Br. |
| 479A | Q² is 5-MeO-3-pyridinyl, R² is Br and R⁴ is Cl. |
| 480A | Q² is 5-MeO-3-pyridinyl, R² is Cl and R⁴ is Br. |
| 481A | Q² is 5-MeO-3-pyridinyl, R² is Cl and R⁴ is Cl. |
| 482A | Q² is 5-MeO-3-pyridinyl, R² is Me and R⁴ is H. |
| 483A | Q² is 5-MeO-3-pyridinyl, R² is Cl and R⁴ is H. |
| 484A | Q² is 5-MeO-3-pyridinyl, R² is Br and R⁴ is H. |
| 485A | Q² is 6-Cl, 5-MeO-3-pyridinyl, R² is Cl and R⁴ is Me. |
| 486A | Q² is 6-Cl, 5-MeO-3-pyridinyl, R² is Cl and R⁴ is CFH₂. |
| 487A | Q² is 6-Cl, 5-MeO-3-pyridinyl, R² is Br and R⁴ is Me. |
| 488A | Q² is 6-Cl, 5-MeO-3-pyridinyl, R² is I and R⁴ is Me. |
| 489A | Q² is 6-Cl, 5-MeO-3-pyridinyl, R² is Me and R⁴ is Me. |
| 490A | Q² is 6-Cl, 5-MeO-3-pyridinyl, R² is Me and R⁴ is Cl. |
| 491A | Q² is 6-Cl, 5-MeO-3-pyridinyl, R² is Me and R⁴ is Br. |
| 492A | Q² is 6-Cl, 5-MeO-3-pyridinyl, R² is Me and R⁴ is I. |
| 493A | Q² is 6-Cl, 5-MeO-3-pyridinyl, R² is Br and R⁴ is Br. |
| 494A | Q² is 6-Cl, 5-MeO-3-pyridinyl, R² is Br and R⁴ is Cl. |
| 495A | Q² is 6-Cl, 5-MeO-3-pyridinyl, R² is Cl and R⁴ is Br. |
| 496A | Q² is 6-Cl, 5-MeO-3-pyridinyl, R² is Cl and R⁴ is Cl. |
| 497A | Q² is 6-Cl, 5-MeO-3-pyridinyl, R² is Me and R⁴ is H. |
| 498A | Q² is 6-Cl, 5-MeO-3-pyridinyl, R² is Cl and R⁴ is H. |
| 499A | Q² is 6-Cl-5-MeO-3-pyridinyl, R² is Br and R⁴ is H. |
| 500A | Q² is 6-Cl-3-pyridazinyl, R² is Cl and R⁴ is Me. |
| 501A | Q² is 6-Cl-3-pyridazinyl, R² is Cl and R⁴ is CFH₂. |
| 502A | Q² is 6-Cl-3-pyridazinyl, R² is Br and R⁴ is Me. |
| 503A | Q² is 6-Cl-3-pyridazinyl, R² is I and R⁴ is Me. |
| 504A | Q² is 6-Cl-3-pyridazinyl, R² is Me and R⁴ is Me. |
| 505A | Q² is 6-Cl-3-pyridazinyl, R² is Me and R⁴ is Cl. |
| 506A | Q² is 6-Cl-3-pyridazinyl, R² is Me and R⁴ is Br. |
| 507A | Q² is 6-Cl-3-pyridazinyl, R² is Me and R⁴ is I. |
| 508A | Q² is 6-Cl-3-pyridazinyl, R² is Br and R⁴ is Br. |
| 509A | Q² is 6-Cl-3-pyridazinyl, R² is Br and R⁴ is Cl. |
| 510A | Q² is 6-Cl-3-pyridazinyl, R² is Cl and R⁴ is Br. |
| 511A | Q² is 6-Cl-3-pyridazinyl, R² is Cl and R⁴ is Cl. |
| 512A | Q² is 6-Cl-3-pyridazinyl, R² is Me and R⁴ is H. |
| 513A | Q² is 6-Cl-3-pyridazinyl, R² is Cl and R⁴ is H. |
| 514A | Q² is 6-Cl-3-pyridazinyl, R² is Br and R⁴ is H. |
| 515A | Q² is 6-Me-3-pyridazinyl, R² is Cl and R⁴ is Me. |
| 516A | Q² is 6-Me-3-pyridazinyl, R² is Cl and R⁴ is CFH₂. |
| 517A | Q² is 6-Me-3-pyridazinyl, R² is Br and R⁴ is Me. |
| 518A | Q² is 6-Me-3-pyridazinyl, R² is I and R⁴ is Me. |
| 519A | Q² is 6-Me-3-pyridazinyl, R² is Me and R⁴ is Me. |
| 520A | Q² is 6-Me-3-pyridazinyl, R² is Me and R⁴ is Cl. |
| 521A | Q² is 6-Me-3-pyridazinyl, R² is Me and R⁴ is Br. |
| 522A | Q² is 6-Me-3-pyridazinyl, R² is Me and R⁴ is I. |
| 523A | Q² is 6-Me-3-pyridazinyl, R² is Br and R⁴ is Br. |
| 524A | Q² is 6-Me-3-pyridazinyl, R² is Br and R⁴ is Cl. |
| 525A | Q² is 6-Me-3-pyridazinyl, R² is Cl and R⁴ is Br. |
| 526A | Q² is 6-Me-3-pyridazinyl, R² is Cl and R⁴ is Cl. |
| 527A | Q² is 6-Me-3-pyridazinyl, R² is Me and R⁴ is H. |
| 528A | Q² is 6-Me-3-pyridazinyl, R² is Cl and R⁴ is H. |
| 529A | Q² is 6-Me-3-pyridazinyl, R² is Br and R⁴ is H. |
| 530A | Q² is 6-MeO-3-pyridazinyl, R² is Cl and R⁴ is Me. |
| 531A | Q² is 6-MeO-3-pyridazinyl, R² is Cl and R⁴ is CFH₂. |
| 532A | Q² is 6-MeO-3-pyridazinyl, R² is Br and R⁴ is Me. |
| 533A | Q² is 6-MeO-3-pyridazinyl, R² is I and R⁴ is Me. |
| 534A | Q² is 6-MeO-3-pyridazinyl, R² is Me and R⁴ is Me. |
| 535A | Q² is 6-MeO-3-pyridazinyl, R² is Me and R⁴ is Cl. |
| 536A | Q² is 6-MeO-3-pyridazinyl, R² is Me and R⁴ is Br. |
| 537A | Q² is 6-MeO-3-pyridazinyl, R² is Me and R⁴ is I. |
| 538A | Q² is 6-MeO-3-pyridazinyl, R² is Br and R⁴ is Br. |
| 539A | Q² is 6-MeO-3-pyridazinyl, R² is Br and R⁴ is Cl. |
| 540A | Q² is 6-MeO-3-pyridazinyl, R² is Cl and R⁴ is Br. |
| 541A | Q² is 6-MeO-3-pyridazinyl, R² is Cl and R⁴ is Cl. |
| 542A | Q² is 6-MeO-3-pyridazinyl, R² is Me and R⁴ is H. |
| 543A | Q² is 6-MeO-3-pyridazinyl, R² is Cl and R⁴ is H. |
| 544A | Q² is 6-MeO-3-pyridazinyl, R² is Br and R⁴ is H. |
| 545A | Q² is 6-CF₃-3-pyridazinyl, R² is Cl and R⁴ is Me. |
| 546A | Q² is 6-CF₃-3-pyridazinyl, R² is Cl and R⁴ is CFH₂. |
| 547A | Q² is 6-CF₃-3-pyridazinyl, R² is Br and R⁴ is Me. |
| 548A | Q² is 6-CF₃-3-pyridazinyl, R² is I and R⁴ is Me. |
| 549A | Q² is 6-CF₃-3-pyridazinyl, R² is Me and R⁴ is Me. |
| 550A | Q² is 6-CF₃-3-pyridazinyl, R² is Me and R⁴ is Cl. |
| 551A | Q² is 6-CF₃-3-pyridazinyl, R² is Me and R⁴ is Br. |
| 552A | Q² is 6-CF₃-3-pyridazinyl, R² is Me and R⁴ is I. |
| 553A | Q² is 6-CF₃-3-pyridazinyl, R² is Br and R⁴ is Br. |
| 554A | Q² is 6-CF₃-3-pyridazinyl, R² is Br and R⁴ is Cl. |
| 555A | Q² is 6-CF₃-3-pyridazinyl, R² is Cl and R⁴ is Br. |
| 556A | Q² is 6-CF₃-3-pyridazinyl, R² is Cl and R⁴ is Cl. |
| 557A | Q² is 6-CF₃-3-pyridazinyl, R² is Me and R⁴ is H. |
| 558A | Q² is 6-CF₃-3-pyridazinyl, R² is Cl and R⁴ is H. |
| 559A | Q² is 6-CF₃-3-pyridazinyl, R² is Br and R⁴ is H. |
| 560A | Q² is 5-Cl-3-pyridazinyl, R² is Cl and R⁴ is Me. |
| 561A | Q² is 5-Cl-3-pyridazinyl, R² is Cl and R⁴ is CFH₂. |
| 562A | Q² is 5-Cl-3-pyridazinyl, R² is Br and R⁴ is Me. |
| 563A | Q² is 5-Cl-3-pyridazinyl, R² is I and R⁴ is Me. |
| 564A | Q² is 5-Cl-3-pyridazinyl, R² is Me and R⁴ is Me. |
| 565A | Q² is 5-Cl-3-pyridazinyl, R² is Me and R⁴ is Cl. |
| 566A | Q² is 5-Cl-3-pyridazinyl, R² is Me and R⁴ is Br. |
| 567A | Q² is 5-Cl-3-pyridazinyl, R² is Me and R⁴ is I. |
| 568A | Q² is 5-Cl-3-pyridazinyl, R² is Br and R⁴ is Br. |
| 569A | Q² is 5-Cl-3-pyridazinyl, R² is Br and R⁴ is Cl. |
| 570A | Q² is 5-Cl-3-pyridazinyl, R² is Cl and R⁴ is Br. |
| 571A | Q² is 5-Cl-3-pyridazinyl, R² is Cl and R⁴ is Cl. |
| 572A | Q² is 5-Cl-3-pyridazinyl, R² is Me and R⁴ is H. |
| 573A | Q² is 5-Cl-3-pyridazinyl, R² is Cl and R⁴ is H. |
| 574A | Q² is 5-Cl-3-pyridazinyl, R² is Br and R⁴ is H. |
| 575A | Q² is 5-F-3-pyridazinyl, R² is Cl and R⁴ is Me. |
| 576A | Q² is 5-F-3-pyridazinyl, R² is Cl and R⁴ is CFH₂. |
| 577A | Q² is 5-F-3-pyridazinyl, R² is Br and R⁴ is Me. |
| 578A | Q² is 5-F-3-pyridazinyl, R² is I and R⁴ is Me. |
| 579A | Q² is 5-F-3-pyridazinyl, R² is Me and R⁴ is Me. |
| 580A | Q² is 5-F-3-pyridazinyl, R² is Me and R⁴ is Cl. |
| 581A | Q² is 5-F-3-pyridazinyl, R² is Me and R⁴ is Br. |
| 582A | Q² is 5-F-3-pyridazinyl, R² is Me and R⁴ is I. |
| 583A | Q² is 5-F-3-pyridazinyl, R² is Br and R⁴ is Br. |
| 584A | Q² is 5-F-3-pyridazinyl, R² is Br and R⁴ is Cl. |
| 585A | Q² is 5-F-3-pyridazinyl, R² is Cl and R⁴ is Br. |
| 586A | Q² is 5-F-3-pyridazinyl, R² is Cl and R⁴ is Cl. |
| 587A | Q² is 5-F-3-pyridazinyl, R² is Me and R⁴ is H. |
| 588A | Q² is 5-F-3-pyridazinyl, R² is Cl and R⁴ is H. |
| 589A | Q² is 5-F-3-pyridazinyl, R² is Br and R⁴ is H. |
| 590A | Q² is 5-MeO-3-pyridazinyl, R² is Cl and R⁴ is Me. |
| 591A | Q² is 5-MeO-3-pyridazinyl, R² is Cl and R⁴ is CFH₂. |
| 592A | Q² is 5-MeO-3-pyridazinyl, R² is Br and R⁴ is Me. |
| 593A | Q² is 5-MeO-3-pyridazinyl, R² is I and R⁴ is Me. |
| 594A | Q² is 5-MeO-3-pyridazinyl, R² is Me and R⁴ is Me. |
| 595A | Q² is 5-MeO-3-pyridazinyl, R² is Me and R⁴ is Cl. |
| 596A | Q² is 5-MeO-3-pyridazinyl, R² is Me and R⁴ is Br. |
| 597A | Q² is 5-MeO-3-pyridazinyl, R² is Me and R⁴ is I. |
| 598A | Q² is 5-MeO-3-pyridazinyl, R² is Br and R⁴ is Br. |
| 599A | Q² is 5-MeO-3-pyridazinyl, R² is Br and R⁴ is Cl. |
| 600A | Q² is 5-MeO-3-pyridazinyl, R² is Cl and R⁴ is Br. |
| 601A | Q² is 5-MeO-3-pyridazinyl, R² is Cl and R⁴ is Cl. |
| 602A | Q² is 5-MeO-3-pyridazinyl, R² is Me and R⁴ is H. |
| 603A | Q² is 5-MeO-3-pyridazinyl, R² is Cl and R⁴ is H. |
| 604A | Q² is 5-MeO-3-pyridazinyl, R² is Br and R⁴ is H. |
| 605A | Q² is 2-Cl-5-pyrimidinyl, R² is Cl and R⁴ is Me. |
| 606A | Q² is 2-Cl-5-pyrimidinyl, R² is Cl and R⁴ is CFH₂. |
| 607A | Q² is 2-Cl-5-pyrimidinyl, R² is Br and R⁴ is Me. |
| 608A | Q² is 2-Cl-5-pyrimidinyl, R² is I and R⁴ is Me. |
| 609A | Q² is 2-Cl-5-pyrimidinyl, R² is Me and R⁴ is Me. |
| 610A | Q² is 2-Cl-5 pyrimidinyl, R² is Me and R⁴ is Cl. |
| 611A | Q² is 2-Cl-5-pyrimidinyl, R² is Me and R⁴ is Br. |
| 612A | Q² is 2-Cl-5-pyrimidinyl, R² is Me and R⁴ is I. |
| 613A | Q² is 2-Cl-5-pyrimidinyl, R² is Br and R⁴ is Br. |
| 614A | Q² is 2-Cl-5-pyrimidinyl, R² is Br and R⁴ is Cl. |
| 615A | Q² is 2-Cl-5-pyrimidinyl, R² is Cl and R⁴ is Br. |
| 616A | Q² is 2-Cl-5-pyrimidinyl, R² is Cl and R⁴ is Cl. |
| 617A | Q² is 2-Cl-5-pyrimidinyl, R² is Me and R⁴ is H. |
| 618A | Q² is 2-Cl-5-pyrimidinyl, R² is Cl and R⁴ is H. |
| 619A | Q² is 2-Cl-5-pyrimidinyl, R² is Br and R⁴ is H. |
| 620A | Q² is 2-Me-5-pyrimidinyl, R² is Cl and R⁴ is Me. |
| 621A | Q² is 2-Me-5-pyrimidinyl, R² is Cl and R⁴ is CFH₂. |
| 622A | Q² is 2-Me-5-pyrimidinyl, R² is Br and R⁴ is Me. |
| 623A | Q² is 2-Me-5-pyrimidinyl, R² is I and R⁴ is Me. |
| 624A | Q² is 2-Me-5-pyrimidinyl, R² is Me and R⁴ is Me. |
| 625A | Q² is 2-Me-5-pyrimidinyl, R² is Me and R⁴ is Cl. |
| 626A | Q² is 2-Me-5-pyrimidinyl, R² is Me and R⁴ is Br. |
| 627A | Q² is 2-Me-5-pyrimidinyl, R² is Me and R⁴ is I. |
| 628A | Q² is 2-Me-5-pyrimidinyl, R² is Br and R⁴ is Br. |
| 629A | Q² is 2-Me-5-pyrimidinyl, R² is Br and R⁴ is Cl. |
| 630A | Q² is 2-Me-5-pyrimidinyl, R² is Cl and R⁴ is Br. |
| 631A | Q² is 2-Me-5-pyrimidinyl, R² is Cl and R⁴ is Cl. |
| 632A | Q² is 2-Me-5-pyrimidinyl, R² is Me and R⁴ is H. |
| 633A | Q² is 2-Me-5-pyrimidinyl, R² is Cl and R⁴ is H. |
| 634A | Q² is 2-Me-5-pyrimidinyl, R² is Br and R⁴ is H. |
| 635A | Q² is 2-MeO-5-pyrimidinyl, R² is Cl and R⁴ is Me. |
| 636A | Q² is 2-MeO-5-pyrimidinyl, R² is Cl and R⁴ is CFH₂. |
| 637A | Q² is 2-MeO-5-pyrimidinyl, R² is Br and R⁴ is Me. |
| 638A | Q² is 2-MeO-5-pyrimidinyl, R² is I and R⁴ is Me. |
| 639A | Q² is 2-MeO-5-pyrimidinyl, R² is Me and R⁴ is Me. |
| 640A | Q² is 2-MeO-5-pyrimidinyl, R² is Me and R⁴ is Cl. |
| 641A | Q² is 2-MeO-5-pyrimidinyl, R² is Me and R⁴ is Br. |
| 642A | Q² is 2-MeO-5-pyrimidinyl, R² is Me and R⁴ is I. |
| 643A | Q² is 2-MeO-5 pyrimidinyl, R² is Br and R⁴ is Br. |
| 644A | Q² is 2-MeO-5-pyrimidinyl, R² is Br and R⁴ is Cl. |
| 645A | Q² is 2-MeO-5-pyrimidinyl, R² is Cl and R⁴ is Br. |
| 646A | Q² is 2-MeO-5-pyrimidinyl, R² is Cl and R⁴ is Cl. |
| 647A | Q² is 2-MeO-5-pyrimidinyl, R² is Me and R⁴ is H. |
| 648A | Q² is 2-MeO-5-pyrimidinyl, R² is Cl and R⁴ is H. |
| 649A | Q² is 2-MeO-5-pyrimidinyl, R² is Br and R⁴ is H. |
| 650A | Q² is 2-CF₃-5-pyrimidinyl, R² is Cl and R⁴ is Me. |
| 651A | Q² is 2-CF₃-5-pyrimidinyl, R² is Cl and R⁴ is CFH₂. |
| 652A | Q² is 2-CF₃-5-pyrimidinyl, R² is Br and R⁴ is Me. |
| 653A | Q² is 2-CF₃-5-pyrimidinyl, R² is I and R⁴ is Me. |
| 654A | Q² is 2-CF₃-5-pyrimidinyl, R² is Me and R⁴ is Me. |
| 655A | Q² is 2-CF₃-5-pyrimidinyl, R² is Me and R⁴ is Cl. |
| 656A | Q² is 2-CF₃-5-pyrimidinyl, R² is Me and R⁴ is Br. |
| 657A | Q² is 2-CF₃-5-pyrimidinyl, R² is Me and R⁴ is I. |
| 658A | Q² is 2-CF₃-5-pyrimidinyl, R² is Br and R⁴ is Br. |
| 659A | Q² is 2-CF₃-5-pyrimidinyl, R² is Br and R⁴ is Cl. |
| 660A | Q² is 2-CF₃-5-pyrimidinyl, R² is Cl and R⁴ is Br. |
| 661A | Q² is 2-CF₃-5-pyrimidinyl, R² is Cl and R⁴ is Cl. |
| 662A | Q² is 2-CF₃-5-pyrimidinyl, R² is Me and R⁴ is H. |
| 663A | Q² is 2-CF₃-5-pyrimidinyl, R² is Cl and R⁴ is H. |
| 664A | Q² is 2-CF₃-5-pyrimidinyl, R² is Br and R⁴ is H. |
| 665A | Q² is 5-Cl-2-pyrimidinyl, R² is Cl and R⁴ is Me. |
| 666A | Q² is 5-Cl-2-pyrimidinyl, R² is Cl and R⁴ is CFH₂. |
| 667A | Q² is 5-Cl-2-pyrimidinyl, R² is Br and R⁴ is Me. |
| 668A | Q² is 5-Cl-2-pyrimidinyl, R² is I and R⁴ is Me. |
| 669A | Q² is 5-Cl-2-pyrimidinyl, R² is Me and R⁴ is Me. |
| 670A | Q² is 5-Cl-2-pyrimidinyl, R² is Me and R⁴ is Cl. |
| 671A | Q² is 5-Cl-2-pyrimidinyl, R² is Me and R⁴ is Br. |
| 672A | Q² is 5-Cl-2-pyrimidinyl, R² is Me and R⁴ is I. |
| 673A | Q² is 5-Cl-2-pyrimidinyl, R² is Br and R⁴ is Br. |
| 674A | Q² is 5-Cl-2-pyrimidinyl, R² is Br and R⁴ is Cl. |
| 675A | Q² is 5-Cl-2-pyrimidinyl, R² is Cl and R⁴ is Br. |
| 676A | Q² is 5-Cl-2-pyrimidinyl, R² is Cl and R⁴ is Cl. |
| 677A | Q² is 5-Cl-2-pyrimidinyl, R² is Me and R⁴ is H. |
| 678A | Q² is 5-Cl-2-pyrimidinyl, R² is Cl and R⁴ is H. |
| 679A | Q² is 5-Cl-2-pyrimidinyl, R² is Br and R⁴ is H. |
| 680A | Q² is 5-Me-2-pyrimidinyl, R² is Cl and R⁴ is Me. |
| 681A | Q² is 5-Me-2-pyrimidinyl, R² is Cl and R⁴ is CFH₂. |
| 682A | Q² is 5-Me-2-pyrimidinyl, R² is Br and R⁴ is Me. |
| 683A | Q² is 5-Me-2-pyrimidinyl, R² is I and R⁴ is Me. |
| 684A | Q² is 5-Me-2-pyrimidinyl, R² is Me and R⁴ is Me. |
| 685A | Q² is 5-Me-2-pyrimidinyl, R² is Me and R⁴ is Cl. |
| 686A | Q² is 5-Me-2-pyrimidinyl, R² is Me and R⁴ is Br. |
| 687A | Q² is 5-Me-2-pyrimidinyl, R² is Me and R⁴ is I. |
| 688A | Q² is 5-Me-2-pyrimidinyl, R² is Br and R⁴ is Br. |
| 689A | Q² is 5-Me-2-pyrimidinyl, R² is Br and R⁴ is Cl. |
| 690A | Q² is 5-Me-2-pyrimidinyl, R² is Cl and R⁴ is Br. |
| 691A | Q² is 5-Me-2-pyrimidinyl, R² is Cl and R⁴ is Cl. |
| 692A | Q² is 5-Me-2-pyrimidinyl, R² is Me and R⁴ is H. |
| 693A | Q² is 5-Me-2-pyrimidinyl, R² is Cl and R⁴ is H. |
| 694A | Q² is 5-Me-2-pyrimidinyl, R² is Br and R⁴ is H. |
| 695A | Q² is 5-MeO-2-pyrimidinyl, R² is Cl and R⁴ is Me. |
| 696A | Q² is 5-MeO-2-pyrimidinyl, R² is Cl and R⁴ is CFH₂. |
| 697A | Q² is 5-MeO-2-pyrimidinyl, R² is Br and R⁴ is Me. |
| 698A | Q² is 5-MeO-2-pyrimidinyl, R² is I and R⁴ is Me. |
| 699A | Q² is 5-MeO-2-pyrimidinyl, R² is Me and R⁴ is Me. |
| 700A | Q² is 5-MeO-2-pyrimidinyl, R² is Me and R⁴ is Cl. |
| 701A | Q² is 5-MeO-2-pyrimidinyl, R² is Me and R⁴ is Br. |
| 702A | Q² is 5-MeO-2-pyrimidinyl, R² is Me and R⁴ is I. |
| 703A | Q² is 5-MeO-2-pyrimidinyl, R² is Br and R⁴ is Br. |
| 704A | Q² is 5-MeO-2-pyrimidinyl, R² is Br and R⁴ is Cl. |
| 705A | Q² is 5-MeO-2-pyrimidinyl, R² is Cl and R⁴ is Br. |
| 706A | Q² is 5-MeO-2-pyrimidinyl, R² is Cl and R⁴ is Cl. |
| 707A | Q² is 5-MeO-2-pyrimidinyl, R² is Me and R⁴ is H. |
| 708A | Q² is 5-MeO-2-pyrimidinyl, R² is Cl and R⁴ is H. |
| 709A | Q² is 5-MeO-2-pyrimidinyl, R² is Br and R⁴ is H. |
| 710A | Q² is 5-Cl-2-pyrimidinyl, R² is Cl and R⁴ is Me. |
| 711A | Q² is 5-Cl-2-pyrimidinyl, R² is Cl and R⁴ is CFH₂. |
| 712A | Q² is 5-Cl-2-pyrimidinyl, R² is Br and R⁴ is Me. |
| 713A | Q² is 5-Cl-2-pyrimidinyl, R² is I and R⁴ is Me. |
| 714A | Q² is 5-Cl-2-pyrimidinyl, R² is Me and R⁴ is Me. |
| 715A | Q² is 5-Cl-2-pyrimidinyl, R² is Me and R⁴ is Cl. |
| 716A | Q² is 5-Cl-2-pyrimidinyl, R² is Me and R⁴ is Br. |
| 717A | Q² is 5-Cl-2-pyrimidinyl, R² is Me and R⁴ is I. |
| 718A | Q² is 5-Cl-2-pyrimidinyl, R² is Br and R⁴ is Br. |
| 719A | Q² is 5-Cl-2-pyrimidinyl, R² is Br and R⁴ is Cl. |
| 720A | Q² is 5-Cl-2-pyrimidinyl, R² is Cl and R⁴ is Br. |
| 721A | Q² is 5-Cl-2-pyrimidinyl, R² is Cl and R⁴ is Cl. |
| 722A | Q² is 5-Cl-2-pyrimidinyl, R² is Me and R⁴ is H. |
| 723A | Q² is 5-Cl-2-pyrimidinyl, R² is Cl and R⁴ is H. |
| 724A | Q² is 5-Cl-2-pyrimidinyl, R² is Br and R⁴ is H. |
| 725A | Q² is 5-CF₃-2-pyrimidinyl, R² is Cl and R⁴ is Me. |
| 726A | Q² is 5-CF₃-2-pyrimidinyl, R² is Cl and R⁴ is CFH₂. |
| 727A | Q² is 5-CF₃-2-pyrimidinyl, R² is Br and R⁴ is Me. |
| 728A | Q² is 5-CF₃-2-pyrimidinyl, R² is I and R⁴ is Me. |
| 729A | Q² is 5-CF₃-2-pyrimidinyl, R² is Me and R⁴ is Me. |
| 730A | Q² is 5-CF₃-2-pyrimidinyl, R² is Me and R⁴ is Cl. |
| 731A | Q² is 5-CF₃-2-pyrimidinyl, R² is Me and R⁴ is Br. |
| 732A | Q² is 5-CF₃-2-pyrimidinyl, R² is Me and R⁴ is I. |
| 733A | Q² is 5-CF₃-2-pyrimidinyl, R² is Br and R⁴ is Br. |
| 734A | Q² is 5-CF₃-2-pyrimidinyl, R² is Br and R⁴ is Cl. |
| 735A | Q² is 5-CF₃-2-pyrimidinyl, R² is Cl and R⁴ is Br. |
| 736A | Q² is 5-CF₃-2-pyrimidinyl, R² is Cl and R⁴ is Cl. |
| 737A | Q² is 5-CF₃-2-pyrimidinyl, R² is Me and R⁴ is H. |
| 738A | Q² is 5-CF₃-2-pyrimidinyl, R² is Cl and R⁴ is H. |
| 739A | Q² is 5-CF₃-2-pyrimidinyl, R² is Br and R⁴ is H. |
| 740A | Q² is 4,6-di-MeO-1,3,4-triazin-2-yl, R² is Cl and R⁴ is Me. |
| 741A | Q² is 4,6-di-MeO-1,3,4-triazin-2-yl, R² is Cl and R⁴ is CFH₂. |
| 742A | Q² is 4,6-di-MeO-1,3,4-triazin-2-yl, R² is Br and R⁴ is Me. |
| 743A | Q² is 4,6-di-MeO-1,3,4-triazin-2-yl, R² is I and R⁴ is Me. |
| 744A | Q² is 4,6-di-MeO-1,3,4-triazin-2-yl, R² is Me and R⁴ is Me. |
| 745A | Q² is 4,6-di-MeO-1,3,4-triazin-2-yl, R² is Me and R⁴ is Cl. |
| 746A | Q² is 4,6-di-MeO-1,3,4-triazin-2-yl, R² is Me and R⁴ is Br. |
| 747A | Q² is 4,6-di-MeO-1,3,4-triazin-2-yl, R² is Me and R⁴ is I. |
| 748A | Q² is 4,6-di-MeO-1,3,4-triazin-2-yl, R² is Br and R⁴ is Br. |
| 749A | Q² is 4,6-di-MeO-1,3,4-triazin-2-yl, R² is Br and R⁴ is Cl. |
| 750A | Q² is 4,6-di-MeO-1,3,4-triazin-2-yl, R² is Cl and R⁴ is Br. |
| 751A | Q² is 4,6-di-MeO-1,3,4-triazin-2-yl, R² is Cl and R⁴ is Cl. |
| 752A | Q² is 4,6-di-MeO-1,3,4-triazin-2-yl, R² is Me and R⁴ is H. |
| 753A | Q² is 4,6-di-MeO-1,3,4-triazin-2-yl, R² is Cl and R⁴ is H. |
| 754A | Q² is 4,6-di-MeO-1,3,4-triazin-2-yl, R² is Br and R⁴ is H. |
| 755A | Q² is 5-Me-2-thienyl, R² is Cl and R⁴ is Me. |
| 756A | Q² is 5-Me-2-thienyl, R² is Cl and R⁴ is CFH₂. |
| 757A | Q² is 5-Me-2-thienyl, R² is Br and R⁴ is Me. |
| 758A | Q² is 5-Me-2-thienyl, R² is I and R⁴ is Me. |
| 759A | Q² is 5-Me-2-thienyl, R² is Me and R⁴ is Me. |
| 760A | Q² is 5-Me-2-thienyl, R² is Me and R⁴ is Cl. |
| 761A | Q² is 5-Me-2-thienyl, R² is Me and R⁴ is Br. |
| 762A | Q² is 5-Me-2-thienyl, R² is Me and R⁴ is I. |
| 763A | Q² is 5-Me-2-thienyl, R² is Br and R⁴ is Br. |
| 764A | Q² is 5-Me-2-thienyl, R² is Br and R⁴ is Cl. |
| 765A | Q² is 5-Me-2-thienyl, R² is Cl and R⁴ is Br. |
| 766A | Q² is 5-Me-2-thienyl, R² is Cl and R⁴ is Cl. |
| 767A | Q² is 5-Me-2-thienyl, R² is Me and R⁴ is H. |
| 768A | Q² is 5-Me-2-thienyl, R² is Cl and R⁴ is H. |
| 769A | Q² is 5-Me-2-thienyl, R² is Br and R⁴ is H. |
| 770A | Q² is 5-Cl-2-thienyl, R² is Cl and R⁴ is Me. |
| 771A | Q² is 5-Cl-2-thienyl, R² is Cl and R⁴ is CFH₂. |
| 772A | Q² is 5-Cl-2-thienyl, R² is Br and R⁴ is Me. |
| 773A | Q² is 5-Cl-2-thienyl, R² is I and R⁴ is Me. |
| 774A | Q² is 5-Cl-2-thienyl, R² is Me and R⁴ is Me. |
| 775A | Q² is 5-Cl-2-thienyl, R² is Me and R⁴ is Cl. |
| 776A | Q² is 5-Cl-2-thienyl, R² is Me and R⁴ is Br. |
| 777A | Q² is 5-Cl-2-thienyl, R² is Me and R⁴ is I. |
| 778A | Q² is 5-Cl-2-thienyl, R² is Br and R⁴ is Br. |
| 779A | Q² is 5-Cl-2-thienyl, R² is Br and R⁴ is Cl. |
| 780A | Q² is 5-Cl-2-thienyl, R² is Cl and R⁴ is Br. |
| 781A | Q² is 5-Cl-2-thienyl, R² is Cl and R⁴ is Cl. |
| 782A | Q² is 5-Cl-2-thienyl, R² is Me and R⁴ is H. |
| 783A | Q² is 5-Cl-2-thienyl, R² is Cl and R⁴ is H. |
| 784A | Q² is 5-Cl-2-thienyl, R² is Br and R⁴ is H. |
| 785A | Q² is 5-F-2-thienyl, R² is Cl and R⁴ is Me. |
| 786A | Q² is 5-F-2-thienyl, R² is Cl and R⁴ is CFH₂. |
| 787A | Q² is 5-F-2-thienyl, R² is Br and R⁴ is Me. |
| 788A | Q² is 5-F-2-thienyl, R² is I and R⁴ is Me. |
| 789A | Q² is 5-F-2-thienyl, R² is Me and R⁴ is Me. |
| 790A | Q² is 5-F-2-thienyl, R² is Me and R⁴ is Cl. |
| 791A | Q² is 5-F-2-thienyl, R² is Me and R⁴ is Br. |
| 792A | Q² is 5-F-2-thienyl, R² is Me and R⁴ is I. |
| 793A | Q² is 5-F-2-thienyl, R² is Br and R⁴ is Br. |
| 794A | Q² is 5-F-2-thienyl, R² is Br and R⁴ is Cl. |
| 795A | Q² is 5-F-2-thienyl, R² is Cl and R⁴ is Br. |
| 796A | Q² is 5-F-2-thienyl, R² is Cl and R⁴ is Cl. |
| 797A | Q² is 5-F-2-thienyl, R² is Me and R⁴ is H. |
| 798A | Q² is 5-F-2-thienyl, R² is Cl and R⁴ is H. |
| 799A | Q² is 5-F-2-thienyl, R² is Br and R⁴ is H. |
| 800A | Q² is 5-Me-3-thienyl, R² is Cl and R⁴ is Me. |
| 801A | Q² is 5-Me-3-thienyl, R² is Cl and R⁴ is CFH₂. |
| 802A | Q² is 5-Me-3-thienyl, R² is Br and R⁴ is Me. |
| 803A | Q² is 5-Me-3-thienyl, R² is I and R⁴ is Me. |
| 804A | Q² is 5-Me-3-thienyl, R² is Me and R⁴ is Me. |
| 805A | Q² is 5-Me-3-thienyl, R² is Me and R⁴ is Cl. |
| 806A | Q² is 5-Me-3-thienyl, R² is Me and R⁴ is Br. |
| 807A | Q² is 5-Me-3-thienyl, R² is Me and R⁴ is I. |
| 808A | Q² is 5-Me-3-thienyl, R² is Br and R⁴ is Br. |
| 809A | Q² is 5-Me-3-thienyl, R² is Br and R⁴ is Cl. |
| 810A | Q² is 5-Me-3-thienyl, R² is Cl and R⁴ is Br. |
| 811A | Q² is 5-Me-3-thienyl, R² is Cl and R⁴ is Cl. |
| 812A | Q² is 5-Me-3-thienyl, R² is Me and R⁴ is H. |
| 813A | Q² is 5-Me-3-thienyl, R² is Cl and R⁴ is H. |
| 814A | Q² is 5-Me-3-thienyl, R² is Br and R⁴ is H. |
| 815A | Q² is 5-Cl-3-thienyl, R² is Cl and R⁴ is Me. |
| 816A | Q² is 5-Cl-3-thienyl, R² is Cl and R⁴ is CFH₂. |
| 817A | Q² is 5-Cl-3-thienyl, R² is Br and R⁴ is Me. |
| 818A | Q² is 5-Cl-3-thienyl, R² is I and R⁴ is Me. |
| 819A | Q² is 5-Cl-3-thienyl, R² is Me and R⁴ is Me. |
| 820A | Q² is 5-Cl-3-thienyl, R² is Me and R⁴ is Cl. |
| 821A | Q² is 5-Cl-3-thienyl, R² is Me and R⁴ is Br. |
| 822A | Q² is 5-Cl-3-thienyl, R² is Me and R⁴ is I. |
| 823A | Q² is 5-Cl-3-thienyl, R² is Br and R⁴ is Br. |
| 824A | Q² is 5-Cl-3-thienyl, R² is Br and R⁴ is Cl. |
| 825A | Q² is 5-Cl-3-thienyl, R² is Cl and R⁴ is Br. |
| 826A | Q² is 5-Cl-3-thienyl, R² is Cl and R⁴ is Cl. |
| 827A | Q² is 5-Cl-3-thienyl, R² is Me and R⁴ is H. |
| 828A | Q² is 5-Cl-3-thienyl, R² is Cl and R⁴ is H. |
| 829A | Q² is 5-Cl-3-thienyl, R² is Br and R⁴ is H. |
| 830A | Q² is 5-F-3-thienyl, R² is Cl and R⁴ is Me. |
| 831A | Q² is 5-F-3-thienyl, R² is Cl and R⁴ is CFH₂. |
| 832A | Q² is 5-F-3-thienyl, R² is Br and R⁴ is Me. |
| 833A | Q² is 5-F-3-thienyl, R² is I and R⁴ is Me. |
| 834A | Q² is 5-F-3-thienyl, R² is Me and R⁴ is Me. |
| 835A | Q² is 5-F-3-thienyl, R² is Me and R⁴ is Cl. |
| 836A | Q² is 5-F-3-thienyl, R² is Me and R⁴ is Br. |
| 837A | Q² is 5-F-3-thienyl, R² is Me and R⁴ is I. |
| 838A | Q² is 5-F-3-thienyl, R² is Br and R⁴ is Br. |
| 839A | Q² is 5-F-3-thienyl, R² is Br and R⁴ is Cl. |
| 840A | Q² is 5-F-3-thienyl, R² is Cl and R⁴ is Br. |
| 841A | Q² is 5-F-3-thienyl, R² is Cl and R⁴ is Cl. |
| 842A | Q² is 5-F-3-thienyl, R² is Me and R⁴ is H. |
| 843A | Q² is 5-F-3-thienyl, R² is Cl and R⁴ is H. |
| 844A | Q² is 5-F-3-thienyl, R² is Br and R⁴ is H. |
| 845A | Q² is 1-Me-1H-pyrazol-3-yl, R² is Cl and R⁴ is Me. |
| 846A | Q² is 1-Me-1H-pyrazol-3-yl, R² is Cl and R⁴ is CFH₂. |
| 847A | Q² is 1-Me-1H-pyrazol-3-yl, R² is Br and R⁴ is Me. |
| 848A | Q² is 1-Me-1H-pyrazol-3-yl, R² is I and R⁴ is Me. |
| 849A | Q² is 1-Me-1H-pyrazol-3-yl, R² is Me and R⁴ is Me. |
| 850A | Q² is 1-Me-1H-pyrazol-3-yl, R² is Me and R⁴ is Cl. |
| 851A | Q² is 1-Me-1H-pyrazol-3-yl, R² is Me and R⁴ is Br. |
| 852A | Q² is 1-Me-1H-pyrazol-3-yl, R² is Me and R⁴ is I. |
| 853A | Q² is 1-Me-1H-pyrazol-3-yl, R² is Br and R⁴ is Br. |
| 854A | Q² is 1-Me-1H-pyrazol-3-yl, R² is Br and R⁴ is Cl. |
| 855A | Q² is 1-Me-1H-pyrazol-3-yl, R² is Cl and R⁴ is Br. |
| 856A | Q² is 1-Me-1H-pyrazol-3-yl, R² is Cl and R⁴ is Cl. |
| 857A | Q² is 1-Me-1H-pyrazol-3-yl, R² is Me and R⁴ is H. |
| 858A | Q² is 1-Me-1H-pyrazol-3-yl, R² is Cl and R⁴ is H. |
| 859A | Q² is 1-Me-1H-pyrazol-3-yl, R² is Br and R⁴ is H. |
| 860A | Q² is 1-Me-1H-pyrazol-4-yl, R² is Cl and R⁴ is Me. |
| 861A | Q² is 1-Me-1H-pyrazol-4-yl, R² is Cl and R⁴ is CFH₂. |
| 862A | Q² is 1-Me-1H-pyrazol-4-yl, R² is Br and R⁴ is Me. |
| 863A | Q² is 1-Me-1H-pyrazol-4-yl, R² is I and R⁴ is Me. |
| 864A | Q² is 1-Me-1H-pyrazol-4-yl, R² is Me and R⁴ is Me. |
| 865A | Q² is 1-Me-1H-pyrazol-4-yl, R² is Me and R⁴ is Cl. |
| 866A | Q² is 1-Me-1H-pyrazol-4-yl, R² is Me and R⁴ is Br. |
| 867A | Q² is 1-Me-1H-pyrazol-4-yl, R² is Me and R⁴ is I. |
| 868A | Q² is 1-Me-1H-pyrazol-4-yl, R² is Br and R⁴ is Br. |
| 869A | Q² is 1-Me-1H-pyrazol-4-yl, R² is Br and R⁴ is Cl. |
| 870A | Q² is 1-Me-1H-pyrazol-4-yl, R² is Cl and R⁴ is Br. |
| 871A | Q² is 1-Me-1H-pyrazol-4-yl, R² is Cl and R⁴ is Cl. |
| 872A | Q² is 1-Me-1H-pyrazol-4-yl, R² is Me and R⁴ is H. |
| 873A | Q² is 1-Me-1H-pyrazol-4-yl, R² is Cl and R⁴ is H. |
| 874A | Q² is 1-Me-1H-pyrazol-4-yl, R² is Br and R⁴ is H. |
| 875A | Q² is 2-Me-5-thiazolyl, R² is Cl and R⁴ is Me. |
| 876A | Q² is 2-Me-5-thiazolyl, R² is Cl and R⁴ is CFH₂. |
| 877A | Q² is 2-Me-5-thiazolyl, R² is Br and R⁴ is Me. |
| 878A | Q² is 2-Me-5-thiazolyl, R² is I and R⁴ is Me. |
| 879A | Q² is 2-Me-5-thiazolyl, R² is Me and R⁴ is Me. |
| 880A | Q² is 2-Me-5-thiazolyl, R² is Me and R⁴ is Cl. |
| 881A | Q² is 2-Me-5-thiazolyl, R² is Me and R⁴ is Br. |
| 882A | Q² is 2-Me-5-thiazolyl, R² is Me and R⁴ is I. |
| 883A | Q² is 2-Me-5-thiazolyl, R² is Br and R⁴ is Br. |
| 884A | Q² is 2-Me-5-thiazolyl, R² is Br and R⁴ is Cl. |
| 885A | Q² is 2-Me-5-thiazolyl, R² is Cl and R⁴ is Br. |
| 886A | Q² is 2-Me-5-thiazolyl, R² is Cl and R⁴ is Cl. |
| 887A | Q² is 2-Me-5-thiazolyl, R² is Me and R⁴ is H. |
| 888A | Q² is 2-Me-5-thiazolyl, R² is Cl and R⁴ is H. |
| 889A | Q² is 2-Me-5-thiazolyl, R² is Br and R⁴ is H. |
| 890A | Q² is 2-Cl-5-thiazolyl, R² is Cl and R⁴ is Me. |
| 891A | Q² is 2-Cl-5-thiazolyl, R² is Cl and R⁴ is CFH₂. |
| 892A | Q² is 2-Cl-5-thiazolyl, R² is Br and R⁴ is Me. |
| 893A | Q² is 2-Cl-5-thiazolyl, R² is I and R⁴ is Me. |
| 894A | Q² is 2-Cl-5-thiazolyl, R² is Me and R⁴ is Me. |
| 895A | Q² is 2-Cl-5-thiazolyl, R² is Me and R⁴ is Cl. |
| 896A | Q² is 2-Cl-5-thiazolyl, R² is Me and R⁴ is Br. |
| 897A | Q² is 2-Cl-5-thiazolyl, R² is Me and R⁴ is I. |
| 898A | Q² is 2-Cl-5-thiazolyl, R² is Br and R⁴ is Br. |
| 899A | Q² is 2-Cl-5-thiazolyl, R² is Br and R⁴ is Cl. |
| 900A | Q² is 2-Cl-5-thiazolyl, R² is Cl and R⁴ is Br. |
| 901A | Q² is 2-Cl-5-thiazolyl, R² is Cl and R⁴ is Cl. |
| 902A | Q² is 2-Cl-5-thiazolyl, R² is Me and R⁴ is H. |
| 903A | Q² is 2-Cl-5-thiazolyl, R² is Cl and R⁴ is H. |
| 904A | Q² is 2-Cl-5-thiazolyl, R² is Br and R⁴ is H. |
| 905A | Q² is 5-Me-3-isothiazolyl, R² is Cl and R⁴ is Me. |
| 906A | Q² is 5-Me-3-isothiazolyl, R² is Cl and R⁴ is CFH₂. |
| 907A | Q² is 5-Me-3-isothiazolyl, R² is Br and R⁴ is Me. |
| 908A | Q² is 5-Me-3-isothiazolyl, R² is I and R⁴ is Me. |
| 909A | Q² is 5-Me-3-isothiazolyl, R² is Me and R⁴ is Me. |
| 910A | Q² is 5-Me-3-isothiazolyl, R² is Me and R⁴ is Cl. |
| 911A | Q² is 5-Me-3-isothiazolyl, R² is Me and R⁴ is Br. |
| 912A | Q² is 5-Me-3-isothiazolyl, R² is Me and R⁴ is I. |
| 913A | Q² is 5-Me-3-isothiazolyl, R² is Br and R⁴ is Br. |
| 914A | Q² is 5-Me-3-isothiazolyl, R² is Br and R⁴ is Cl. |
| 915A | Q² is 5-Me-3-isothiazolyl, R² is Cl and R⁴ is Br. |
| 916A | Q² is 5-Me-3-isothiazolyl, R² is Cl and R⁴ is Cl. |
| 917A | Q² is 5-Me-3-isothiazolyl, R² is Me and R⁴ is H. |
| 918A | Q² is 5-Me-3-isothiazolyl, R² is Cl and R⁴ is H. |
| 919A | Q² is 5-Me-3-isothiazolyl, R² is Br and R⁴ is H. |
| 920A | Q² is 5-Cl-3-isothiazolyl, R² is Cl and R⁴ is Me. |
| 921A | Q² is 5-Cl-3-isothiazolyl, R² is Cl and R⁴ is CFH₂. |
| 922A | Q² is 5-Cl-3-isothiazolyl, R² is Br and R⁴ is Me. |
| 923A | Q² is 5-Cl-3-isothiazolyl, R² is I and R⁴ is Me. |
| 924A | Q² is 5-Cl-3-isothiazolyl, R² is Me and R⁴ is Me. |
| 925A | Q² is 5-Cl-3-isothiazolyl, R² is Me and R⁴ is Cl. |
| 926A | Q² is 5-Cl-3-isothiazolyl, R² is Me and R⁴ is Br. |
| 927A | Q² is 5-Cl-3-isothiazolyl, R² is Me and R⁴ is I. |
| 928A | Q² is 5-Cl-3-isothiazolyl, R² is Br and R⁴ is Br. |
| 929A | Q² is 5-Cl-3-isothiazolyl, R² is Br and R⁴ is Cl. |
| 930A | Q² is 5-Cl-3-isothiazolyl, R² is Cl and R⁴ is Br. |
| 931A | Q² is 5-Cl-3-isothiazolyl, R² is Cl and R⁴ is Cl. |
| 932A | Q² is 5-Cl-3-isothiazolyl, R² is Me and R⁴ is H. |
| 933A | Q² is 5-Cl-3-isothiazolyl, R² is Cl and R⁴ is H. |
| 934A | Q² is 5-Cl-3-isothiazolyl, R² is Br and R⁴ is H. |

TABLE 2

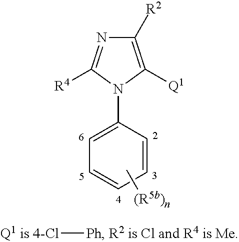

| (R^{5b})ₙ | (R^{5b})ₙ | (R^{5b})ₙ | (R^{5b})ₙ | (R^{5b})ₙ |
|---|---|---|---|---|
| 2,6-di-F | 2,4-di-Cl | 4-CN, 2,6-di-F | 2-CF₃, 4-F | 2-Cl, 4-NO₂ |
| 2,4,6-tri-F | 2-Cl, 4,6-di-F | 2,6-di-F, 4-Me | 2-CF₂HO, 4-F | 2-NO₂, 4-F |
| 2,3,6-tri-F | 2-Cl, 6-F | 2-Cl, 5-CF₃ | 2-CN, 6-F | 2,3,4,5,6-penta-F |
| 2,4,5-tri-F | 2-Br, 6-F | 2-Cl, 4-Me | 2,5-di-Cl | 2,5-di-Cl, 4-F |
| 2,3,4-tri-F | 2-F, 6-CF₃ | 2-Cl, 4-MeO | 2-CF₃, 4-MeO | 2,3-di-Cl, 4-F |
| 2-Cl, 4-F | 2-F, 6-CF₂HO | 2-Br, 4-MeO | 2-F, 6-Me | 2-Cl, 5-CN |
| 2-Br-4-F | 2-I, 4-F | 2,6-di-F, 3-Cl | 2,6-di-F, 3-Me | 2,4-di-F, 5-CN |
| 2,4-di-F | 4-Cl, 2,6-di-F | 2,6-di-F, 3-CN | 2-CF₃ | 2-Cl, 6-F, 3-MeO |
| 2,6-di-Cl | 2,6-di-F, 4-MeO | 2,6-di-F, 3-MeO | 2-CF₃O | |
| 2,6-di-F, 4-EtO | 2-Cl, 6-F, 4-MeO | 2,6-di-F, 4-CF₂HO | 2,6-di-F, 4-NO₂ | |
| 2-Cl, 3,6-di-F | 2-Cl, 6-F, 5-MeO | 2,6-di-F, 3-CF₂HO | 2,6-di-F, 3-EtO | |

The present disclosure also includes Tables 1B through 934B, each of which is constructed the same as Table 2 above except that the row heading in Table 2 (i.e. “Q¹ is 4-Cl-Ph, R² is Cl and R⁴ is Me”) is replaced with the respective row heading shown below. Thus, for example, in Table 1B the row heading is “Q¹ is 4-Cl-Ph, R² is Cl and R⁴ is CFH₂”, and (R^{5b})ₙ is as defined in Table 2 above. Tables 2B through 934B are constructed similarly.

| Table | Row Heading |
|---|---|
| 1B | Q¹ is 4-Cl-Ph, R² is Cl and R⁴ is CFH₂. |
| 2B | Q¹ is 4-Cl-Ph, R² is Br and R⁴ is Me. |
| 3B | Q¹ is 4-Cl-Ph, R² is I and R⁴ is Me. |
| 4B | Q¹ is 4-Cl-Ph, R² is Me and R⁴ is Me. |
| 5B | Q¹ is 4-Cl-Ph, R² is Me and R⁴ is Cl. |
| 6B | Q¹ is 4-Cl-Ph, R² is Me and R⁴ is Br. |
| 7B | Q¹ is 4-Cl-Ph, R² is Me and R⁴ is I. |
| 8B | Q¹ is 4-Cl-Ph, R² is Me and R⁴ is MeO. |
| 9B | Q¹ is 4-Cl-Ph, R² is MeO and R⁴ is Me. |
| 10B | Q¹ is 4-Cl-Ph, R² is Br and R⁴ is Br. |
| 11B | Q¹ is 4-Cl-Ph, R² is Br and R⁴ is Cl. |
| 12B | Q¹ is 4-Cl-Ph, R² is Cl and R⁴ is Br. |
| 13B | Q¹ is 4-Cl-Ph, R² is Cl and R⁴ is Cl. |
| 14B | Q¹ is 4-Cl-Ph, R² is Me and R⁴ is MeS. |
| 15B | Q¹ is 4-Cl-Ph, R² is MeS and R⁴ is Me. |
| 16B | Q¹ is 4-Cl-Ph, R² is Et and R⁴ is Br. |
| 17B | Q¹ is 4-Cl-Ph, R² is Et and R⁴ is Cl. |
| 18B | Q¹ is 4-Cl-Ph, R² is Et and R⁴ is Me. |
| 19B | Q¹ is 4-Cl-Ph, R² is Me and R⁴ is Et. |
| 20B | Q¹ is 4-Cl-Ph, R² is Cl and R⁴ is Et. |
| 21B | Q¹ is 4-Cl-Ph, R² is Me and R⁴ is CN. |
| 22B | Q¹ is 4-Cl-Ph, R² is Me and R⁴ is H. |
| 23B | Q¹ is 4-Cl-Ph, R² is Cl and R⁴ is H. |
| 24B | Q¹ is 4-Cl-Ph, R² is Br and R⁴ is H. |
| 25B | Q¹ is 3-Cl-Ph, R² is Cl and R⁴ is Me. |
| 26B | Q¹ is 3-Cl-Ph, R² is Cl and R⁴ is CFH₂. |
| 27B | Q¹ is 3-Cl-Ph, R² is Br and R⁴ is Me. |
| 28B | Q¹ is 3-Cl-Ph, R² is I and R⁴ is Me. |
| 29B | Q¹ is 3-Cl-Ph, R² is Me and R⁴ is Me. |
| 30B | Q¹ is 3-Cl-Ph, R² is Me and R⁴ is Cl. |
| 31B | Q¹ is 3-Cl-Ph, R² is Me and R⁴ is Br. |
| 32B | Q¹ is 3-Cl-Ph, R² is Me and R⁴ is I. |
| 33B | Q¹ is 3-Cl-Ph, R² is Br and R⁴ is Br. |
| 34B | Q¹ is 3-Cl-Ph, R² is Br and R⁴ is Cl. |
| 35B | Q¹ is 3-Cl-Ph, R² is Cl and R⁴ is Br. |
| 36B | Q¹ is 3-Cl-Ph, R² is Cl and R⁴ is Cl. |
| 37B | Q¹ is 3-Cl-Ph, R² is Me and R⁴ is H. |
| 38B | Q¹ is 3-Cl-Ph, R² is Cl and R⁴ is H. |
| 39B | Q¹ is 3-Cl-Ph, R² is Br and R⁴ is H |
| 40B | Q¹ is 4-F-Ph, R² is Cl and R⁴ is Me. |
| 41B | Q¹ is 4-F-Ph, R² is Cl and R⁴ is CFH₂. |
| 42B | Q¹ is 4-F-Ph, R² is Br and R⁴ is Me. |
| 43B | Q¹ is 4-F-Ph, R² is I and R⁴ is Me. |
| 44B | Q¹ is 4-F-Ph, R² is Me and R⁴ is Me. |
| 45B | Q¹ is 4-F-Ph, R² is Me and R⁴ is Cl. |
| 46B | Q¹ is 4-F-Ph, R² is Me and R⁴ is Br. |
| 47B | Q¹ is 4-F-Ph, R² is Me and R⁴ is I. |
| 48B | Q¹ is 4-F-Ph, R² is Br and R⁴ is Br. |
| 49B | Q¹ is 4-F-Ph, R² is Br and R⁴ is Cl. |
| 50B | Q¹ is 4-F-Ph, R² is Cl and R⁴ is Br. |
| 51B | Q¹ is 4-F-Ph, R² is Cl and R⁴ is Cl. |
| 52B | Q¹ is 4-F-Ph, R² is Me and R⁴ is H. |
| 53B | Q¹ is 4-F-Ph, R² is Cl and R⁴ is H. |
| 54B | Q¹ is 4-F-Ph, R² is Br and R⁴ is H. |
| 55B | Q¹ is 3-F-Ph, R² is Cl and R⁴ is Me. |
| 56B | Q¹ is 3-F-Ph, R² is Cl and R⁴ is CFH₂. |
| 57B | Q¹ is 3-F-Ph, R² is Br and R⁴ is Me. |
| 58B | Q¹ is 3-F-Ph, R² is I and R⁴ is Me. |
| 59B | Q¹ is 3-F-Ph, R² is Me and R⁴ is Me. |
| 60B | Q¹ is 3-F-Ph, R² is Me and R⁴ is Cl. |
| 61B | Q¹ is 3-F-Ph, R² is Me and R⁴ is Br. |
| 62B | Q¹ is 3-F-Ph, R² is Me and R⁴ is I. |
| 63B | Q¹ is 3-F-Ph, R² is Br and R⁴ is Br. |
| 64B | Q¹ is 3-F-Ph, R² is Br and R⁴ is Cl. |
| 65B | Q¹ is 3-F-Ph, R² is Cl and R⁴ is Br. |
| 66B | Q¹ is 3-F-Ph, R² is Cl and R⁴ is Cl. |
| 67B | Q¹ is 3-F-Ph, R² is Me and R⁴ is H. |
| 68B | Q¹ is 3-F-Ph, R² is Cl and R⁴ is H. |
| 69B | Q¹ is 3-F-Ph, R² is Br and R⁴ is H |
| 70B | Q¹ is 3-CF₂HO-Ph, R² is Cl and R⁴ is Me. |
| 71B | Q¹ is 3-CF₂HO-Ph, R² is Cl and R⁴ is CH₂F. |
| 72B | Q¹ is 3-CF₂HO-Ph, R² is Br and R⁴ is Me. |
| 73B | Q¹ is 3-CF₂HO-Ph, R² is I and R⁴ is Me. |
| 74B | Q¹ is 3-CF₂HO-Ph, R² is Me and R⁴ is Me. |
| 75B | Q¹ is 3-CF₂HO-Ph, R² is Me and R⁴ is Cl. |
| 76B | Q¹ is 3-CF₂HO-Ph, R² is Me and R⁴ is Br. |
| 77B | Q¹ is 3-CF₂HO-Ph, R² is Me and R⁴ is I. |
| 78B | Q¹ is 3-CF₂HO-Ph, R² is Br and R⁴ is Br. |
| 79B | Q¹ is 3-CF₂HO-Ph, R² is Br and R⁴ is Cl. |
| 80B | Q¹ is 3-CF₂HO-Ph, R² is Cl and R⁴ is Br. |
| 81B | Q¹ is 3-CF₂HO-Ph, R² is Cl and R⁴ is Cl. |
| 82B | Q¹ is 3-CF₂HO-Ph, R² is Me and R⁴ is H. |
| 83B | Q¹ is 3-CF₂HO-Ph, R² is Cl and R⁴ is H. |
| 84B | Q¹ is 3-CF₂HO-Ph, R² is Br and R⁴ is H |
| 85B | Q¹ is 4-Me-Ph, R² is Cl and R⁴ is Me. |
| 86B | Q¹ is 4-Me-Ph, R² is Cl and R⁴ is CFH₂. |
| 87B | Q¹ is 4-Me-Ph, R² is Br and R⁴ is Me. |
| 88B | Q¹ is 4-Me-Ph, R² is I and R⁴ is Me. |
| 89B | Q¹ is 4-Me-Ph, R² is Me and R⁴ is Me. |
| 90B | Q¹ is 4-Me-Ph, R² is Me and R⁴ is Cl. |
| 91B | Q¹ is 4-Me-Ph, R² is Me and R⁴ is Br. |
| 92B | Q¹ is 4-Me-Ph, R² is Me and R⁴ is I. |
| 93B | Q¹ is 4-Me-Ph, R² is Br and R⁴ is Br. |
| 94B | Q¹ is 4-Me-Ph, R² is Br and R⁴ is Cl. |
| 95B | Q¹ is 4-Me-Ph, R² is Cl and R⁴ is Br. |
| 96B | Q¹ is 4-Me-Ph, R² is Cl and R⁴ is Cl. |
| 97B | Q¹ is 4-Me-Ph, R² is Me and R⁴ is H. |
| 98B | Q¹ is 4-Me-Ph, R² is Cl and R⁴ is H. |
| 99B | Q¹ is 4-Me-Ph, R² is Br and R⁴ is H. |
| 100B | Q¹ is 3-Me-Ph, R² is Cl and R⁴ is Me. |
| 101B | Q¹ is 3-Me-Ph, R² is Cl and R⁴ is CFH₂. |
| 102B | Q¹ is 3-Me-Ph, R² is Br and R⁴ is Me. |
| 103B | Q¹ is 3-Me-Ph, R² is I and R⁴ is Me. |
| 104B | Q¹ is 3-Me-Ph, R² is Me and R⁴ is Me. |
| 105B | Q¹ is 3-Me-Ph, R² is Me and R⁴ is Cl. |
| 106B | Q¹ is 3-Me-Ph, R² is Me and R⁴ is Br. |
| 107B | Q¹ is 3-Me-Ph, R² is Me and R⁴ is I. |
| 108B | Q¹ is 3-Me-Ph, R² is Br and R⁴ is Br. |
| 109B | Q¹ is 3-Me-Ph, R² is Br and R⁴ is Cl. |
| 110B | Q¹ is 3-Me-Ph, R² is Cl and R⁴ is Br. |
| 111B | Q¹ is 3-Me-Ph, R² is Cl and R⁴ is Cl. |
| 112B | Q¹ is 3-Me-Ph, R² is Me and R⁴ is H. |
| 113B | Q¹ is 3-Me-Ph, R² is Cl and R⁴ is H. |
| 114B | Q¹ is 3-Me-Ph, R² is Br and R⁴ is H. |
| 115B | Q¹ is 4-Et-Ph, R² is Cl and R⁴ is Me. |
| 116B | Q¹ is 4-Et-Ph, R² is Cl and R⁴ is CFH₂. |
| 117B | Q¹ is 4-Et-Ph, R² is Br and R⁴ is Me. |
| 118B | Q¹ is 4-Et-Ph, R² is I and R⁴ is Me. |
| 119B | Q¹ is 4-Et-Ph, R² is Me and R⁴ is Me. |
| 120B | Q¹ is 4-Et-Ph, R² is Me and R⁴ is Cl. |
| 121B | Q¹ is 4-Et-Ph, R² is Me and R⁴ is Br. |
| 122B | Q¹ is 4-Et-Ph, R² is Me and R⁴ is I. |
| 123B | Q¹ is 4-Et-Ph, R² is Br and R⁴ is Br. |
| 124B | Q¹ is 4-Et-Ph, R² is Br and R⁴ is Cl. |
| 125B | Q¹ is 4-Et-Ph, R² is Cl and R⁴ is Br. |
| 126B | Q¹ is 4-Et-Ph, R² is Cl and R⁴ is Cl. |
| 127B | Q¹ is 4-Et-Ph, R² is Me and R⁴ is H. |
| 128B | Q¹ is 4-Et-Ph, R² is Cl and R⁴ is H. |
| 129B | Q¹ is 4-Et-Ph, R² is Br and R⁴ is H. |
| 130B | Q¹ is 4-Cl, 3-F-Ph, R² is Cl and R⁴ is Me. |
| 131B | Q¹ is 4-Cl, 3-F-Ph, R² is Cl and R⁴ is CFH₂. |
| 132B | Q¹ is 4-Cl, 3-F-Ph, R² is Br and R⁴ is Me. |
| 133B | Q¹ is 4-Cl, 3-F-Ph, R² is I and R⁴ is Me. |
| 134B | Q¹ is 4-Cl, 3-F-Ph, R² is Me and R⁴ is Me. |
| 135B | Q¹ is 4-Cl, 3-F-Ph, R² is Me and R⁴ is Cl. |
| 136B | Q¹ is 4-Cl, 3-F-Ph, R² is Me and R⁴ is Br. |
| 137B | Q¹ is 4-Cl, 3-F-Ph, R² is Me and R⁴ is I. |
| 138B | Q¹ is 4-Cl, 3-F-Ph, R² is Br and R⁴ is Br. |
| 139B | Q¹ is 4-Cl, 3-F-Ph, R² is Br and R⁴ is Cl. |
| 140B | Q¹ is 4-Cl, 3-F-Ph, R² is Cl and R⁴ is Br. |
| 141B | Q¹ is 4-Cl, 3-F-Ph, R² is Cl and R⁴ is Cl. |
| 142B | Q¹ is 4-Cl, 3-F-Ph, R² is Me and R⁴ is H. |
| 143B | Q¹ is 4-Cl, 3-F-Ph R² is Cl and R⁴ is H. |
| 144B | Q¹ is 4-Cl, 3-F-Ph, R² is Br and R⁴ is H. |
| 145B | Q¹ is 2-Cl, 4-F-Ph, R² is Cl and R⁴ is Me. |
| 146B | Q¹ is 2-Cl, 4-F-Ph, R² is Cl and R⁴ is CFH₂. |
| 147B | Q¹ is 2-Cl, 4-F-Ph, R² is Br and R⁴ is Me. |
| 148B | Q¹ is 2-Cl, 4-F-Ph, R² is I and R⁴ is Me. |
| 149B | Q¹ is 2-Cl, 4-F-Ph, R² is Me and R⁴ is Me. |
| 150B | Q¹ is 2-Cl, 4-F-Ph, R² is Me and R⁴ is Cl. |
| 151B | Q¹ is 2-Cl, 4-F-Ph, R² is Me and R⁴ is Br. |
| 152B | Q¹ is 2-Cl, 4-F-Ph, R² is Me and R⁴ is I. |
| 153B | Q¹ is 2-Cl, 4-F-Ph, R² is Br and R⁴ is Br. |
| 154B | Q¹ is 2-Cl, 4-F-Ph, R² is Br and R⁴ is Cl. |
| 155B | Q¹ is 2-Cl, 4-F-Ph, R² is Cl and R⁴ is Br. |
| 156B | Q¹ is 2-Cl, 4-F-Ph, R² is Cl and R⁴ is Cl. |
| 157B | Q¹ is 2-Cl, 4-F-Ph, R² is Me and R⁴ is H. |
| 158B | Q¹ is 2-Cl, 4-F-Ph, R² is Cl and R⁴ is H. |
| 159B | Q¹ is 2-Cl, 4-F-Ph, R² is Br and R⁴ is H. |
| 160B | Q¹ is 4-F, 3-Me-Ph, R² is Cl and R⁴ is Me. |
| 161B | Q¹ is 4-F, 3-Me-Ph, R² is Cl and R⁴ is CFH₂. |
| 162B | Q¹ is 4-F, 3-Me-Ph, R² is Br and R⁴ is Me. |
| 163B | Q¹ is 4-F, 3-Me-Ph, R² is I and R⁴ is Me. |
| 164B | Q¹ is 4-F, 3-Me-Ph, R² is Me and R⁴ is Me. |
| 165B | Q¹ is 4-F, 3-Me-Ph, R² is Me and R⁴ is Cl. |
| 166B | Q¹ is 4-F, 3-Me-Ph, R² is Me and R⁴ is Br. |
| 167B | Q¹ is 4-F, 3-Me-Ph, R² is Me and R⁴ is I. |
| 168B | Q¹ is 4-F, 3-Me-Ph, R² is Br and R⁴ is Br. |
| 169B | Q¹ is 4-F, 3-Me-Ph, R² is Br and R⁴ is Cl. |
| 170B | Q¹ is 4-F, 3-Me-Ph, R² is Cl and R⁴ is Br. |
| 171B | Q¹ is 4-F, 3-Me-Ph, R² is Cl and R⁴ is Cl. |
| 172B | Q¹ is 4-F, 3-Me-Ph, R² is Me and R⁴ is H. |
| 173B | Q¹ is 4-F, 3-Me-Ph, R² is Cl and R⁴ is H. |
| 174B | Q¹ is 4-F, 3-Me-Ph, R² is Br and R⁴ is H. |
| 175B | Q¹ is 3,4-di-F-Ph, R² is Cl and R⁴ is Me. |
| 176B | Q¹ is 3,4-di-F-Ph, R² is Cl and R⁴ is CFH₂. |
| 177B | Q¹ is 3,4-di-F-Ph, R² is Br and R⁴ is Me. |
| 178B | Q¹ is 3,4-di-F-Ph, R² is I and R⁴ is Me. |
| 179B | Q¹ is 3,4-di-F-Ph, R² is Me and R⁴ is Me. |
| 180B | Q¹ is 3,4-di-F-Ph, R² is Me and R⁴ is Cl. |
| 181B | Q¹ is 3,4-di-F-Ph, R² is Me and R⁴ is Br. |
| 182B | Q¹ is 3,4-di-F-Ph, R² is Me and R⁴ is I. |
| 183B | Q¹ is 3,4-di-F-Ph, R² is Br and R⁴ is Br. |
| 184B | Q¹ is 3,4-di-F-Ph, R² is Br and R⁴ is Cl. |
| 185B | Q¹ is 3,4-di-F-Ph, R² is Cl and R⁴ is Br. |
| 186B | Q¹ is 3,4-di-F-Ph, R² is Cl and R⁴ is Cl. |
| 187B | Q¹ is 3,4-di-F-Ph, R² is Me and R⁴ is H. |
| 188B | Q¹ is 3,4-di-F-Ph, R² is Cl and R⁴ is H. |
| 189B | Q¹ is 3,4-di-F-Ph, R² is Br and R⁴ is H. |
| 190B | Q¹ is 3,4-di-Cl-Ph, R² is Cl and R⁴ is Me. |
| 191B | Q¹ is 3,4-di-Cl-Ph, R² is Cl and R⁴ is CFH₂. |
| 192B | Q¹ is 3,4-di-Cl-Ph, R² is Br and R⁴ is Me. |
| 193B | Q¹ is 3,4-di-Cl-Ph, R² is I and R⁴ is Me. |
| 194B | Q¹ is 3,4-di-Cl-Ph, R² is Me and R⁴ is Me. |
| 195B | Q¹ is 3,4-di-Cl-Ph, R² is Me and R⁴ is Cl. |
| 196B | Q¹ is 3,4-di-Cl-Ph, R² is Me and R⁴ is Br. |
| 197B | Q¹ is 3,4-di-Cl-Ph, R² is Me and R⁴ is I. |
| 198B | Q¹ is 3,4-di-Cl-Ph, R² is Br and R⁴ is Br. |
| 199B | Q¹ is 3,4-di-Cl-Ph, R² is Br and R⁴ is Cl. |
| 200B | Q¹ is 3,4-di-Cl-Ph, R² is Cl and R⁴ is Br. |
| 201B | Q¹ is 3,4-di-Cl-Ph, R² is Cl and R⁴ is Cl. |
| 202B | Q¹ is 3,4-di-Cl-Ph, R² is Me and R⁴ is H. |
| 203B | Q¹ is 3,4-di-Cl-Ph, R² is Cl and R⁴ is H. |
| 204B | Q¹ is 3,4-di-Cl-Ph, R² is Br and R⁴ is H. |
| 205B | Q¹ is 3,5-di-MeO-Ph, R² is Cl and R⁴ is Me. |
| 206B | Q¹ is 3,5-di-MeO-Ph, R² is Cl and R⁴ is CFH₂. |
| 207B | Q¹ is 3,5-di-MeO-Ph, R² is Br and R⁴ is Me. |
| 208B | Q¹ is 3,5-di-MeO-Ph, R² is I and R⁴ is Me. |
| 209B | Q¹ is 3,5-di-MeO-Ph, R² is Me and R⁴ is Me. |
| 210B | Q¹ is 3,5-di-MeO-Ph, R² is Me and R⁴ is Cl. |
| 211B | Q¹ is 3,5-di-MeO-Ph, R² is Me and R⁴ is Br. |
| 212B | Q¹ is 3,5-di-MeO-Ph, R² is Me and R⁴ is I. |
| 213B | Q¹ is 3,5-di-MeO-Ph, R² is Br and R⁴ is Br. |
| 214B | Q¹ is 3,5-di-MeO-Ph, R² is Br and R⁴ is Cl. |
| 215B | Q¹ is 3,5-di-MeO-Ph, R² is Cl and R⁴ is Br. |
| 216B | Q¹ is 3,5-di-MeO-Ph, R² is Cl and R⁴ is Cl. |
| 217B | Q¹ is 3,5-di-MeO-Ph, R² is Me and R⁴ is H. |
| 218B | Q¹ is 3,5-di-MeO-Ph, R² is Cl and R⁴ is H. |
| 219B | Q¹ is 3,5-di-MeO-Ph, R² is Br and R⁴ is H. |
| 220B | Q¹ is 2-Cl, 3,5-di-MeO-Ph, R² is Cl and R⁴ is Me. |
| 221B | Q¹ is 2-Cl, 3,5-di-MeO-Ph, R² is Cl and R⁴ is CFH₂. |
| 222B | Q¹ is 2-Cl, 3,5-di-MeO-Ph, R² is Br and R⁴ is Me. |
| 223B | Q¹ is 2-Cl, 3,5-di-MeO-Ph, R² is I and R⁴ is Me. |
| 224B | Q¹ is 2-Cl, 3,5-di-MeO-Ph, R² is Me and R⁴ is Me. |
| 225B | Q¹ is 2-Cl, 3,5-di-MeO-Ph, R² is Me and R⁴ is Cl. |
| 226B | Q¹ is 2-Cl, 3,5-di-MeO-Ph, R² is Me and R⁴ is Br. |
| 227B | Q¹ is 2-Cl, 3,5-di-MeO-Ph, R² is Me and R⁴ is I. |
| 228B | Q¹ is 2-Cl, 3,5-di-MeO-Ph, R² is Br and R⁴ is Br. |
| 229B | Q¹ is 2-Cl, 3,5-di-MeO-Ph, R² is Br and R⁴ is Cl. |
| 230B | Q¹ is 2-Cl, 3,5-di-MeO-Ph, R² is Cl and R⁴ is Br. |
| 231B | Q¹ is 2-Cl, 3,5-di-MeO-Ph, R² is Cl and R⁴ is Cl. |
| 232B | Q¹ is 2-Cl, 3,5-di-MeO-Ph, R² is Me and R⁴ is H. |
| 233B | Q¹ is 2-Cl, 3,5-di-MeO-Ph, R² is Cl and R⁴ is H. |
| 234B | Q¹ is 2-Cl, 3,5-di-MeO-Ph, R² is Br and R⁴ is H. |
| 235B | Q¹ is 4-Cl, 3,5-di-MeO-Ph, R² is Cl and R⁴ is Me. |
| 236B | Q¹ is 4-Cl, 3,5-di-MeO-Ph, R² is Cl and R⁴ is CFH₂. |
| 237B | Q¹ is 4-Cl, 3,5-di-MeO-Ph, R² is Br and R⁴ is Me. |
| 238B | Q¹ is 4-Cl, 3,5-di-MeO-Ph, R² is I and R⁴ is Me. |
| 239B | Q¹ is 4-Cl, 3,5-di-MeO-Ph, R² is Me and R⁴ is Me. |
| 240B | Q¹ is 4-Cl, 3,5-di-MeO-Ph, R² is Me and R⁴ is Cl. |
| 241B | Q¹ is 4-Cl, 3,5-di-MeO-Ph, R² is Me and R⁴ is Br. |
| 242B | Q¹ is 4-Cl, 3,5-di-MeO-Ph, R² is Me and R⁴ is I. |
| 243B | Q¹ is 4-Cl, 3,5-di-MeO-Ph, R² is Br and R⁴ is Br. |
| 244B | Q¹ is 4-Cl, 3,5-di-MeO-Ph, R² is Br and R⁴ is Cl. |
| 245B | Q¹ is 4-Cl, 3,5-di-MeO-Ph, R² is Cl and R⁴ is Br. |
| 246B | Q¹ is 4-Cl, 3,5-di-MeO-Ph, R² is Cl and R⁴ is Cl. |
| 247B | Q¹ is 4-Cl, 3,5-di-MeO-Ph, R² is Me and R⁴ is H. |
| 248B | Q¹ is 4-Cl, 3,5-di-MeO-Ph, R² is Cl and R⁴ is H. |
| 249B | Q¹ is 4-Cl, 3,5-di-MeO-Ph, R² is Br and R⁴ is H. |
| 250B | Q¹ is 4-Cl-Bn, R² is Cl and R⁴ is Me. |
| 251B | Q¹ is 4-Cl-Bn, R² is Cl and R⁴ is CFH₂. |
| 252B | Q¹ is 4-Cl-Bn, R² is Br and R⁴ is Me. |
| 253B | Q¹ is 4-Cl-Bn, R² is I and R⁴ is Me. |
| 254B | Q¹ is 4-Cl-Bn, R² is Me and R⁴ is Me. |
| 255B | Q¹ is 4-Cl-Bn, R² is Me and R⁴ is Cl. |
| 256B | Q¹ is 4-Cl-Bn, R² is Me and R⁴ is Br. |
| 257B | Q¹ is 4-Cl-Bn, R² is Me and R⁴ is I. |
| 258B | Q¹ is 4-Cl-Bn, R² is Br and R⁴ is Br. |
| 259B | Q¹ is 4-Cl-Bn, R² is Br and R⁴ is Cl. |
| 260B | Q¹ is 4-Cl-Bn, R² is Cl and R⁴ is Br. |
| 261B | Q¹ is 4-Cl-Bn, R² is Cl and R⁴ is Cl. |
| 262B | Q¹ is 4-Cl-Bn, R² is Me and R⁴ is H. |
| 263B | Q¹ is 4-Cl-Bn, R² is Cl and R⁴ is H. |
| 264B | Q¹ is 4-Cl-Bn, R² is Br and R⁴ is H. |
| 265B | Q¹ is 4-F-Bn, R² is Cl and R⁴ is Me. |
| 266B | Q¹ is 4-F-Bn, R² is Cl and R⁴ is CFH₂. |
| 267B | Q¹ is 4-F-Bn, R² is Br and R⁴ is Me. |
| 268B | Q¹ is 4-F-Bn, R² is I and R⁴ is Me. |
| 269B | Q¹ is 4-F-Bn, R² is Me and R⁴ is Me. |
| 270B | Q¹ is 4-F-Bn, R² is Me and R⁴ is Cl. |
| 271B | Q¹ is 4-F-Bn, R² is Me and R⁴ is Br. |
| 272B | Q¹ is 4-F-Bn, R² is Me and R⁴ is I. |
| 273B | Q¹ is 4-F-Bn, R² is Br and R⁴ is Br. |
| 274B | Q¹ is 4-F-Bn, R² is Br and R⁴ is Cl. |
| 275B | Q¹ is 4-F-Bn, R² is Cl and R⁴ is Br. |
| 276B | Q¹ is 4-F-Bn, R² is Cl and R⁴ is Cl. |
| 277B | Q¹ is 4-F-Bn, R² is Me and R⁴ is H. |
| 278B | Q¹ is 4-F-Bn, R² is Cl and R⁴ is H. |
| 279B | Q¹ is 4-F-Bn, R² is Br and R⁴ is H. |
| 280B | Q¹ is 6-Cl-3-pyridinyl, R² is Cl and R⁴ is Me. |
| 281B | Q¹ is 6-Cl-3-pyridinyl, R² is Cl and R⁴ is CFH₂. |
| 282B | Q¹ is 6-Cl-3-pyridinyl, R² is Br and R⁴ is Me. |
| 283B | Q¹ is 6-Cl-3-pyridinyl, R² is I and R⁴ is Me. |
| 284B | Q¹ is 6-Cl-3-pyridinyl, R² is Me and R⁴ is Me. |
| 285B | Q¹ is 6-Cl-3-pyridinyl, R² is Me and R⁴ is Cl. |
| 286B | Q¹ is 6-Cl-3-pyridinyl, R² is Me and R⁴ is Br. |
| 287B | Q¹ is 6-Cl-3-pyridinyl, R² is Me and R⁴ is I. |
| 288B | Q¹ is 6-Cl-3-pyridinyl, R² is Me and R⁴ is MeO. |
| 289B | Q¹ is 6-Cl-3-pyridinyl, R² is MeO and R⁴ is Me. |
| 290B | Q¹ is 6-Cl-3-pyridinyl, R² is Br and R⁴ is Br. |
| 291B | Q¹ is 6-Cl-3-pyridinyl, R² is Br and R⁴ is Cl. |
| 292B | Q¹ is 6-Cl-3-pyridinyl, R² is Cl and R⁴ is Br. |
| 293B | Q¹ is 6-Cl-3-pyridinyl, R² is Cl and R⁴ is Cl. |
| 294B | Q¹ is 6-Cl-3-pyridinyl, R² is Me and R⁴ is MeS. |
| 295B | Q¹ is 6-Cl-3-pyridinyl, R² is MeS and R⁴ is Me. |
| 296B | Q¹ is 6-Cl-3-pyridinyl, R² is Et and R⁴ is Br. |
| 297B | Q¹ is 6-Cl-3-pyridinyl, R² is Et and R⁴ is Cl. |
| 298B | Q¹ is 6-Cl-3-pyridinyl, R² is Et and R⁴ is Me. |
| 299B | Q¹ is 6-Cl-3-pyridinyl, R² is Me and R⁴ is Et. |
| 300B | Q¹ is 6-Cl-3-pyridinyl, R² is Cl and R⁴ is Et. |
| 301B | Q¹ is 6-Cl-3-pyridinyl, R² is Me and R⁴ is CN. |
| 302B | Q¹ is 6-Cl-3-pyridinyl, R² is Me and R⁴ is H. |
| 303B | Q¹ is 6-Cl-3-pyridinyl, R² is Cl and R⁴ is H. |
| 304B | Q¹ is 6-Cl-3-pyridinyl, R² is Br and R⁴ is H. |
| 305B | Q¹ is 6-Me-3-pyridinyl, R² is Cl and R⁴ is Me. |
| 306B | Q¹ is 6-Me-3-pyridinyl, R² is Cl and R⁴ is CFH₂. |
| 307B | Q¹ is 6-Me-3-pyridinyl, R² is Br and R⁴ is Me. |
| 308B | Q¹ is 6-Me-3-pyridinyl, R² is I and R⁴ is Me. |
| 309B | Q¹ is 6-Me-3-pyridinyl, R² is Me and R⁴ is Me. |
| 310B | Q¹ is 6-Me-3-pyridinyl, R² is Me and R⁴ is Cl. |
| 311B | Q¹ is 6-Me-3-pyridinyl, R² is Me and R⁴ is Br. |
| 312B | Q¹ is 6-Me-3-pyridinyl, R² is Me and R⁴ is I. |
| 313B | Q¹ is 6-Me-3-pyridinyl, R² is Br and R⁴ is Br. |
| 314B | Q¹ is 6-Me-3-pyridinyl, R² is Br and R⁴ is Cl. |
| 315B | Q¹ is 6-Me-3-pyridinyl, R² is Cl and R⁴ is Br. |
| 316B | Q¹ is 6-Me-3-pyridinyl, R² is Cl and R⁴ is Cl. |
| 317B | Q¹ is 6-Me-3-pyridinyl, R² is Me and R⁴ is H. |
| 318B | Q¹ is 6-Me-3-pyridinyl, R² is Cl and R⁴ is H. |
| 319B | Q¹ is 6-Me-3-pyridinyl, R² is Br and R⁴ is H. |
| 320B | Q¹ is 6-MeO-3-pyridinyl, R² is Cl and R⁴ is Me. |
| 321B | Q¹ is 6-MeO-3-pyridinyl, R² is Cl and R⁴ is CFH₂. |
| 322B | Q¹ is 6-MeO-3-pyridinyl, R² is Br and R⁴ is Me. |
| 323B | Q¹ is 6-MeO-3-pyridinyl, R² is I and R⁴ is Me. |
| 324B | Q¹ is 6-MeO-3-pyridinyl, R² is Me and R⁴ is Me. |
| 325B | Q¹ is 6-MeO-3-pyridinyl, R² is Me and R⁴ is Cl. |
| 326B | Q¹ is 6-MeO-3-pyridinyl, R² is Me and R⁴ is Br. |
| 327B | Q¹ is 6-MeO-3-pyridinyl, R² is Me and R⁴ is I. |
| 328B | Q¹ is 6-MeO-3-pyridinyl, R² is Br and R⁴ is Br. |
| 329B | Q¹ is 6-MeO-3-pyridinyl, R² is Br and R⁴ is Cl. |
| 330B | Q¹ is 6-MeO-3-pyridinyl, R² is Cl and R⁴ is Br. |
| 331B | Q¹ is 6-MeO-3-pyridinyl, R² is Cl and R⁴ is Cl. |
| 332B | Q¹ is 6-MeO-3-pyridinyl, R² is Me and R⁴ is H. |
| 333B | Q¹ is 6-MeO-3-pyridinyl, R² is Cl and R⁴ is H. |
| 334B | Q¹ is 6-MeO-3-pyridinyl, R² is Br and R⁴ is H. |
| 335B | Q¹ is 6-CF₃-3-pyridinyl, R² is Cl and R⁴ is Me. |
| 336B | Q¹ is 6-CF₃-3-pyridinyl, R² is Cl and R⁴ is CFH₂. |
| 337B | Q¹ is 6-CF₃-3-pyridinyl, R² is Br and R⁴ is Me. |
| 338B | Q¹ is 6-CF₃-3-pyridinyl, R² is I and R⁴ is Me. |
| 339B | Q¹ is 6-CF₃-3-pyridinyl, R² is Me and R⁴ is Me. |
| 340B | Q¹ is 6-CF₃-3-pyridinyl, R² is Me and R⁴ is Cl. |
| 341B | Q¹ is 6-CF₃-3-pyridinyl, R² is Me and R⁴ is Br. |
| 342B | Q¹ is 6-CF₃-3-pyridinyl, R² is Me and R⁴ is I. |
| 343B | Q¹ is 6-CF₃-3-pyridinyl, R² is Br and R⁴ is Br. |
| 344B | Q¹ is 6-CF₃-3-pyridinyl, R² is Br and R⁴ is Cl. |
| 345B | Q¹ is 6-CF₃-3-pyridinyl, R² is Cl and R⁴ is Br. |
| 346B | Q¹ is 6-CF₃-3-pyridinyl, R² is Cl and R⁴ is Cl. |
| 347B | Q¹ is 6-CF₃-3-pyridinyl, R² is Me and R⁴ is H. |
| 348B | Q¹ is 6-CF₃-3-pyridinyl, R² is Cl and R⁴ is H. |
| 349B | Q¹ is 6-CF₃-3-pyridinyl, R² is Br and R⁴ is H. |
| 350B | Q¹ is 6-Br-3-pyridinyl, R² is Cl and R⁴ is Me. |
| 351B | Q¹ is 6-Br-3-pyridinyl, R² is Cl and R⁴ is CFH₂. |
| 352B | Q¹ is 6-Br-3-pyridinyl, R² is Br and R⁴ is Me. |
| 353B | Q¹ is 6-Br-3-pyridinyl, R² is I and R⁴ is Me. |
| 354B | Q¹ is 6-Br-3-pyridinyl, R² is Me and R⁴ is Me. |
| 355B | Q¹ is 6-Br-3-pyridinyl, R² is Me and R⁴ is Cl. |
| 356B | Q¹ is 6-Br-3-pyridinyl, R² is Me and R⁴ is Br. |
| 357B | Q¹ is 6-Br-3-pyridinyl, R² is Me and R⁴ is I. |
| 358B | Q¹ is 6-Br-3-pyridinyl, R² is Br and R⁴ is Br. |
| 359B | Q¹ is 6-Br-3-pyridinyl, R² is Br and R⁴ is Cl. |
| 360B | Q¹ is 6-Br-3-pyridinyl, R² is Cl and R⁴ is Br. |
| 361B | Q¹ is 6-Br-3-pyridinyl, R² is Cl and R⁴ is Cl. |
| 362B | Q¹ is 6-Br-3-pyridinyl, R² is Me and R⁴ is H. |
| 363B | Q¹ is 6-Br-3-pyridinyl, R² is Cl and R⁴ is H. |
| 364B | Q¹ is 6-Br-3-pyridinyl, R² is Br and R⁴ is H. |
| 365B | Q¹ is 6-F-3-pyridinyl, R² is Cl and R⁴ is Me. |
| 366B | Q¹ is 6-F-3-pyridinyl, R² is Cl and R⁴ is CFH₂. |
| 367B | Q¹ is 6-F-3-pyridinyl, R² is Br and R⁴ is Me. |
| 368B | Q¹ is 6-F-3-pyridinyl, R² is I and R⁴ is Me. |
| 369B | Q¹ is 6-F-3-pyridinyl, R² is Me and R⁴ is Me. |
| 370B | Q¹ is 6-F-3-pyridinyl, R² is Me and R⁴ is Cl. |
| 371B | Q¹ is 6-F-3-pyridinyl, R² is Me and R⁴ is Br. |
| 372B | Q¹ is 6-F-3-pyridinyl, R² is Me and R⁴ is I. |
| 373B | Q¹ is 6-F-3-pyridinyl, R² is Br and R⁴ is Br. |
| 374B | Q¹ is 6-F-3-pyridinyl, R² is Br and R⁴ is Cl. |
| 375B | Q¹ is 6-F-3-pyridinyl, R² is Cl and R⁴ is Br. |
| 376B | Q¹ is 6-F-3-pyridinyl, R² is Cl and R⁴ is Cl. |
| 377B | Q¹ is 6-F-3-pyridinyl, R² is Me and R⁴ is H. |
| 378B | Q¹ is 6-F-3-pyridinyl, R² is Cl and R⁴ is H. |
| 379B | Q¹ is 6-F-3-pyridinyl, R² is Br and R⁴ is H. |
| 380B | Q¹ is 2-Cl, 6-Me-4-pyridinyl, R² is Cl and R⁴ is Me. |
| 381B | Q¹ is 2-Cl, 6-Me-4-pyridinyl, R² is Cl and R⁴ is CFH₂. |
| 382B | Q¹ is 2-Cl, 6-Me-4-pyridinyl, R² is Br and R⁴ is Me. |
| 383B | Q¹ is 2-Cl, 6-Me-4-pyridinyl, R² is I and R⁴ is Me. |
| 384B | Q¹ is 2-Cl, 6-Me-4-pyridinyl, R² is Me and R⁴ is Me. |
| 385B | Q¹ is 2-Cl, 6-Me-4-pyridinyl, R² is Me and R⁴ is Cl. |
| 386B | Q¹ is 2-Cl, 6-Me-4-pyridinyl, R² is Me and R⁴ is Br. |
| 387B | Q¹ is 2-Cl, 6-Me-4-pyridinyl, R² is Me and R⁴ is I. |
| 388B | Q¹ is 2-Cl, 6-Me-4-pyridinyl, R² is Br and R⁴ is Br. |
| 389B | Q¹ is 2-Cl, 6-Me-4-pyridinyl, R² is Br and R⁴ is Cl. |
| 390B | Q¹ is 2-Cl, 6-Me-4-pyridinyl, R² is Cl and R⁴ is Br. |
| 391B | Q¹ is 2-Cl, 6-Me-4-pyridinyl, R² is Cl and R⁴ is Cl. |
| 392B | Q¹ is 2-Cl, 6-Me-4-pyridinyl, R² is Me and R⁴ is H. |
| 393B | Q¹ is 2-Cl, 6-Me-4-pyridinyl, R² is Cl and R⁴ is H. |
| 394B | Q¹ is 2-Cl, 6-Me-4-pyridinyl, R² is Br and R⁴ is H. |
| 395B | Q¹ is 2-Cl, 6-MeO-3-pyridinyl, R² is Cl and R⁴ is Me. |
| 396B | Q¹ is 2-Cl, 6-MeO-3-pyridinyl, R² is Cl and R⁴ is CFH₂. |
| 397B | Q¹ is 2-Cl, 6-MeO-3-pyridinyl, R² is Br and R⁴ is Me. |
| 398B | Q¹ is 2-Cl, 6-MeO-3-pyridinyl, R² is I and R⁴ is Me. |
| 399B | Q¹ is 2-Cl, 6-MeO-3-pyridinyl, R² is Me and R⁴ is Me. |
| 400B | Q¹ is 2-Cl, 6-MeO-3-pyridinyl, R² is Me and R⁴ is Cl. |
| 401B | Q¹ is 2-Cl, 6-MeO-3-pyridinyl, R² is Me and R⁴ is Br. |
| 402B | Q¹ is 2-Cl, 6-MeO-3-pyridinyl, R² is Me and R⁴ is I. |
| 403B | Q¹ is 2-Cl, 6-MeO-3-pyridinyl, R² is Br and R⁴ is Br. |
| 404B | Q¹ is 2-Cl, 6-MeO-3-pyridinyl, R² is Br and R⁴ is Cl. |
| 405B | Q¹ is 2-Cl, 6-MeO-3-pyridinyl, R² is Cl and R⁴ is Br. |
| 406B | Q¹ is 2-Cl, 6-MeO-3-pyridinyl, R² is Cl and R⁴ is Cl. |
| 407B | Q¹ is 2-Cl, 6-MeO-3-pyridinyl, R² is Me and R⁴ is H. |
| 408B | Q¹ is 2-Cl, 6-MeO-3-pyridinyl, R² is Cl and R⁴ is H. |
| 409B | Q¹ is 2-Cl, 6-MeO-3-pyridinyl, R² is Br and R⁴ is H. |
| 410B | Q¹ is 2-Cl, 6-CF₃-3-pyridinyl, R² is Cl and R⁴ is Me. |
| 411B | Q¹ is 2-Cl, 6-CF₃-3-pyridinyl, R² is Cl and R⁴ is CFH₂. |
| 412B | Q¹ is 2-Cl, 6-CF₃-3-pyridinyl, R² is Br and R⁴ is Me. |
| 413B | Q¹ is 2-Cl, 6-CF₃-3-pyridinyl, R² is I and R⁴ is Me. |
| 414B | Q¹ is 2-Cl, 6-CF₃-3-pyridinyl, R² is Me and R⁴ is Me. |
| 415B | Q¹ is 2-Cl, 6-CF₃-3-pyridinyl, R² is Me and R⁴ is Cl. |
| 416B | Q¹ is 2-Cl, 6-CF₃-3-pyridinyl, R² is Me and R⁴ is Br. |
| 417B | Q¹ is 2-Cl, 6-CF₃-3-pyridinyl, R² is Me and R⁴ is I. |
| 418B | Q¹ is 2-Cl, 6-CF₃-3-pyridinyl, R² is Br and R⁴ is Br. |
| 419B | Q¹ is 2-Cl, 6-CF₃-3-pyridinyl, R² is Br and R⁴ is Cl. |
| 420B | Q¹ is 2-Cl, 6-CF₃-3-pyridinyl, R² is Cl and R⁴ is Br. |
| 421B | Q¹ is 2-Cl, 6-CF₃-3-pyridinyl, R² is Cl and R⁴ is Cl. |
| 422B | Q¹ is 2-Cl, 6-CF₃-3-pyridinyl, R² is Me and R⁴ is H. |
| 423B | Q¹ is 2-Cl, 6-CF₃-3-pyridinyl, R² is Cl and R⁴ is H. |
| 424B | Q¹ is 2-Cl, 6-CF₃-3-pyridinyl, R² is Br and R⁴ is H. |
| 425B | Q¹ is 5-Cl-3-pyridinyl, R² is Cl and R⁴ is Me. |
| 426B | Q¹ is 5-Cl-3-pyridinyl, R² is Cl and R⁴ is CFH₂. |
| 427B | Q¹ is 5-Cl-3-pyridinyl, R² is Br and R⁴ is Me. |
| 428B | Q¹ is 5-Cl-3-pyridinyl, R² is I and R⁴ is Me. |
| 429B | Q¹ is 5-Cl-3-pyridinyl, R² is Me and R⁴ is Me. |
| 430B | Q¹ is 5-Cl-3-pyridinyl, R² is Me and R⁴ is Cl. |
| 431B | Q¹ is 5-Cl-3-pyridinyl, R² is Me and R⁴ is Br. |
| 432B | Q¹ is 5-Cl-3-pyridinyl, R² is Me and R⁴ is I. |
| 433B | Q¹ is 5-Cl-3-pyridinyl, R² is Br and R⁴ is Br. |
| 434B | Q¹ is 5-Cl-3-pyridinyl, R² is Br and R⁴ is Cl. |
| 435B | Q¹ is 5-Cl-3-pyridinyl, R² is Cl and R⁴ is Br. |
| 436B | Q¹ is 5-Cl-3-pyridinyl, R² is Cl and R⁴ is Cl. |
| 437B | Q¹ is 5-Cl-3-pyridinyl, R² is Me and R⁴ is H. |
| 438B | Q¹ is 5-Cl-3-pyridinyl, R² is Cl and R⁴ is H. |
| 439B | Q¹ is 5-Cl-3-pyridinyl, R² is Br and R⁴ is H. |
| 440B | Q¹ is 5-F-3-pyridinyl, R² is Cl and R⁴ is Me. |
| 441B | Q¹ is 5-F-3-pyridinyl, R² is Cl and R⁴ is CFH₂. |
| 442B | Q¹ is 5-F-3-pyridinyl, R² is Br and R⁴ is Me. |
| 443B | Q¹ is 5-F-3-pyridinyl, R² is I and R⁴ is Me. |
| 444B | Q¹ is 5-F-3-pyridinyl, R² is Me and R⁴ is Me. |
| 445B | Q¹ is 5-F-3-pyridinyl, R² is Me and R⁴ is Cl. |
| 446B | Q¹ is 5-F-3-pyridinyl, R² is Me and R⁴ is Br. |
| 447B | Q¹ is 5-F-3-pyridinyl, R² is Me and R⁴ is I. |
| 448B | Q¹ is 5-F-3-pyridinyl, R² is Br and R⁴ is Br. |
| 449B | Q¹ is 5-F-3-pyridinyl, R² is Br and R⁴ is Cl. |
| 450B | Q¹ is 5-F-3-pyridinyl, R² is Cl and R⁴ is Br. |
| 451B | Q¹ is 5-F-3-pyridinyl, R² is Cl and R⁴ is Cl. |
| 452B | Q¹ is 5-F-3-pyridinyl, R² is Me and R⁴ is H. |
| 453B | Q¹ is 5-F-3-pyridinyl, R² is Cl and R⁴ is H. |
| 454B | Q¹ is 5-F-3-pyridinyl, R² is Br and R⁴ is H. |
| 455B | Q¹ is 5-Me-3-pyridinyl, R² is Cl and R⁴ is Me. |
| 456B | Q¹ is 5-Me-3-pyridinyl, R² is Cl and R⁴ is CFH₂. |
| 457B | Q¹ is 5-Me-3-pyridinyl, R² is Br and R⁴ is Me. |
| 458B | Q¹ is 5-Me-3-pyridinyl, R² is I and R⁴ is Me. |
| 459B | Q¹ is 5-Me-3-pyridinyl, R² is Me and R⁴ is Me. |
| 460B | Q¹ is 5-Me-3-pyridinyl, R² is Me and R⁴ is Cl. |
| 461B | Q¹ is 5-Me-3-pyridinyl, R² is Me and R⁴ is Br. |
| 462B | Q¹ is 5-Me-3-pyridinyl, R² is Me and R⁴ is I. |
| 463B | Q¹ is 5-Me-3-pyridinyl, R² is Br and R⁴ is Br. |
| 464B | Q¹ is 5-Me-3-pyridinyl, R² is Br and R⁴ is Cl. |
| 465B | Q¹ is 5-Me-3-pyridinyl, R² is Cl and R⁴ is Br. |
| 466B | Q¹ is 5-Me-3-pyridinyl, R² is Cl and R⁴ is Cl. |
| 467B | Q¹ is 5-Me-3-pyridinyl, R² is Me and R⁴ is H. |
| 468B | Q¹ is 5-Me-3-pyridinyl, R² is Cl and R⁴ is H. |
| 469B | Q¹ is 5-Me-3-pyridinyl, R² is Br and R⁴ is H. |
| 470B | Q¹ is 5-MeO-3-pyridinyl, R² is Cl and R⁴ is Me. |
| 471B | Q¹ is 5-MeO-3-pyridinyl, R² is Cl and R⁴ is CFH₂. |
| 472B | Q¹ is 5-MeO-3-pyridinyl, R² is Br and R⁴ is Me. |
| 473B | Q¹ is 5-MeO-3-pyridinyl, R² is I and R⁴ is Me. |
| 474B | Q¹ is 5-MeO-3-pyridinyl, R² is Me and R⁴ is Me. |
| 475B | Q¹ is 5-MeO-3-pyridinyl, R² is Me and R⁴ is Cl. |
| 476B | Q¹ is 5-MeO-3-pyridinyl, R² is Me and R⁴ is Br. |
| 477B | Q¹ is 5-MeO-3-pyridinyl, R² is Me and R⁴ is I. |
| 478B | Q¹ is 5-MeO-3-pyridinyl, R² is Br and R⁴ is Br. |
| 479B | Q¹ is 5-MeO-3-pyridinyl, R² is Br and R⁴ is Cl. |
| 480B | Q¹ is 5-MeO-3-pyridinyl, R² is Cl and R⁴ is Br. |
| 481B | Q¹ is 5-MeO-3-pyridinyl, R² is Cl and R⁴ is Cl. |
| 482B | Q¹ is 5-MeO-3-pyridinyl, R² is Me and R⁴ is H. |
| 483B | Q¹ is 5-MeO-3-pyridinyl, R² is Cl and R⁴ is H. |
| 484B | Q¹ is 5-MeO-3-pyridinyl, R² is Br and R⁴ is H. |
| 485B | Q¹ is 6-Cl, 5-MeO-3-pyridinyl, R² is Cl and R⁴ is Me. |
| 486B | Q¹ is 6-Cl, 5-MeO-3-pyridinyl, R² is Cl and R⁴ is CFH₂. |
| 487B | Q¹ is 6-Cl, 5-MeO-3-pyridinyl, R² is Br and R⁴ is Me. |
| 488B | Q¹ is 6-Cl, 5-MeO-3-pyridinyl, R² is I and R⁴ is Me. |
| 489B | Q¹ is 6-Cl, 5-MeO-3-pyridinyl, R² is Me and R⁴ is Me. |
| 490B | Q¹ is 6-Cl, 5-MeO-3-pyridinyl, R² is Me and R⁴ is Cl. |
| 491B | Q¹ is 6-Cl, 5-MeO-3-pyridinyl, R² is Me and R⁴ is Br. |
| 492B | Q¹ is 6-Cl, 5-MeO-3-pyridinyl, R² is Me and R⁴ is I. |
| 493B | Q¹ is 6-Cl, 5-MeO-3-pyridinyl, R² is Br and R⁴ is Br. |
| 494B | Q¹ is 6-Cl, 5-MeO-3-pyridinyl, R² is Br and R⁴ is Cl. |
| 495B | Q¹ is 6-Cl, 5-MeO-3-pyridinyl, R² is Cl and R⁴ is Br. |
| 496B | Q¹ is 6-Cl, 5-MeO-3-pyridinyl, R² is Cl and R⁴ is Cl. |
| 497B | Q¹ is 6-Cl, 5-MeO-3-pyridinyl, R² is Me and R⁴ is H. |
| 498B | Q¹ is 6-Cl, 5-MeO-3-pyridinyl, R² is Cl and R⁴ is H. |
| 499B | Q¹ is 6-Cl-5-MeO-3-pyridinyl, R² is Br and R⁴ is H. |
| 500B | Q¹ is 6-Cl-3-pyridazinyl, R² is Cl and R⁴ is Me. |
| 501B | Q¹ is 6-Cl-3-pyridazinyl, R² is Cl and R⁴ is CFH₂. |
| 502B | Q¹ is 6-Cl-3-pyridazinyl, R² is Br and R⁴ is Me. |
| 503B | Q¹ is 6-Cl-3-pyridazinyl, R² is I and R⁴ is Me. |
| 504B | Q¹ is 6-Cl-3-pyridazinyl, R² is Me and R⁴ is Me. |
| 505B | Q¹ is 6-Cl-3-pyridazinyl, R² is Me and R⁴ is Cl. |
| 506B | Q¹ is 6-Cl-3-pyridazinyl, R² is Me and R⁴ is Br. |
| 507B | Q¹ is 6-Cl-3-pyridazinyl, R² is Me and R⁴ is I. |
| 508B | Q¹ is 6-Cl-3-pyridazinyl, R² is Br and R⁴ is Br. |
| 509B | Q¹ is 6-Cl-3-pyridazinyl, R² is Br and R⁴ is Cl. |
| 510B | Q¹ is 6-Cl-3-pyridazinyl, R² is Cl and R⁴ is Br. |
| 511B | Q¹ is 6-Cl-3-pyridazinyl, R² is Cl and R⁴ is Cl. |
| 512B | Q¹ is 6-Cl-3-pyridazinyl, R² is Me and R⁴ is H. |
| 513B | Q¹ is 6-Cl-3-pyridazinyl, R² is Cl and R⁴ is H. |
| 514B | Q¹ is 6-Cl-3-pyridazinyl, R² is Br and R⁴ is H. |
| 515B | Q¹ is 6-Me-3-pyridazinyl, R² is Cl and R⁴ is Me. |
| 516B | Q¹ is 6-Me-3-pyridazinyl, R² is Cl and R⁴ is CFH₂. |
| 517B | Q¹ is 6-Me-3-pyridazinyl, R² is Br and R⁴ is Me. |
| 518B | Q¹ is 6-Me-3-pyridazinyl, R² is I and R⁴ is Me. |
| 519B | Q¹ is 6-Me-3-pyridazinyl, R² is Me and R⁴ is Me. |
| 520B | Q¹ is 6-Me-3-pyridazinyl, R² is Me and R⁴ is Cl. |
| 521B | Q¹ is 6-Me-3-pyridazinyl, R² is Me and R⁴ is Br. |
| 522B | Q¹ is 6-Me-3-pyridazinyl, R² is Me and R⁴ is I. |
| 523B | Q¹ is 6-Me-3-pyridazinyl, R² is Br and R⁴ is Br. |
| 524B | Q¹ is 6-Me-3-pyridazinyl, R² is Br and R⁴ is Cl. |
| 525B | Q¹ is 6-Me-3-pyridazinyl, R² is Cl and R⁴ is Br. |
| 526B | Q¹ is 6-Me-3-pyridazinyl, R² is Cl and R⁴ is Cl. |
| 527B | Q¹ is 6-Me-3-pyridazinyl, R² is Me and R⁴ is H. |
| 528B | Q¹ is 6-Me-3-pyridazinyl, R² is Cl and R⁴ is H. |
| 529B | Q¹ is 6-Me-3-pyridazinyl, R² is Br and R⁴ is H. |
| 530B | Q¹ is 6-MeO-3-pyridazinyl, R² is Cl and R⁴ is Me. |
| 531B | Q¹ is 6-MeO-3-pyridazinyl, R² is Cl and R⁴ is CFH₂. |
| 532B | Q¹ is 6-MeO-3-pyridazinyl, R² is Br and R⁴ is Me. |
| 533B | Q¹ is 6-MeO-3-pyridazinyl, R² is I and R⁴ is Me. |
| 534B | Q¹ is 6-MeO-3-pyridazinyl, R² is Me and R⁴ is Me. |
| 535B | Q¹ is 6-MeO-3-pyridazinyl, R² is Me and R⁴ is Cl. |
| 536B | Q¹ is 6-MeO-3-pyridazinyl, R² is Me and R⁴ is Br. |
| 537B | Q¹ is 6-MeO-3-pyridazinyl, R² is Me and R⁴ is I. |
| 538B | Q¹ is 6-MeO-3-pyridazinyl, R² is Br and R⁴ is Br. |
| 539B | Q¹ is 6-MeO-3-pyridazinyl, R² is Br and R⁴ is Cl. |
| 540B | Q¹ is 6-MeO-3-pyridazinyl, R² is Cl and R⁴ is Br. |
| 541B | Q¹ is 6-MeO-3-pyridazinyl, R² is Cl and R⁴ is Cl. |
| 542B | Q¹ is 6-MeO-3-pyridazinyl, R² is Me and R⁴ is H. |
| 543B | Q¹ is 6-MeO-3-pyridazinyl, R² is Cl and R⁴ is H. |
| 544B | Q¹ is 6-MeO-3-pyridazinyl, R² is Br and R⁴ is H. |
| 545B | Q¹ is 6-CF₃-3-pyridazinyl, R² is Cl and R⁴ is Me. |
| 546B | Q¹ is 6-CF₃-3-pyridazinyl, R² is Cl and R⁴ is CFH₂. |
| 547B | Q¹ is 6-CF₃-3-pyridazinyl, R² is Br and R⁴ is Me. |
| 548B | Q¹ is 6-CF₃-3-pyridazinyl, R² is I and R⁴ is Me. |
| 549B | Q¹ is 6-CF₃-3-pyridazinyl, R² is Me and R⁴ is Me. |
| 550B | Q¹ is 6-CF₃-3-pyridazinyl, R² is Me and R⁴ is Cl. |
| 551B | Q¹ is 6-CF₃-3-pyridazinyl, R² is Me and R⁴ is Br. |
| 552B | Q¹ is 6-CF₃-3-pyridazinyl, R² is Me and R⁴ is I. |
| 553B | Q¹ is 6-CF₃-3-pyridazinyl, R² is Br and R⁴ is Br. |
| 554B | Q¹ is 6-CF₃-3-pyridazinyl, R² is Br and R⁴ is Cl. |
| 555B | Q¹ is 6-CF₃-3-pyridazinyl, R² is Cl and R⁴ is Br. |
| 556B | Q¹ is 6-CF₃-3-pyridazinyl, R² is Cl and R⁴ is Cl. |
| 557B | Q¹ is 6-CF₃-3-pyridazinyl, R² is Me and R⁴ is H. |
| 558B | Q¹ is 6-CF₃-3-pyridazinyl, R² is Cl and R⁴ is H. |
| 559B | Q¹ is 6-CF₃-3-pyridazinyl, R² is Br and R⁴ is H. |
| 560B | Q¹ is 5-Cl-3-pyridazinyl, R² is Cl and R⁴ is Me. |
| 561B | Q¹ is 5-Cl-3-pyridazinyl, R² is Cl and R⁴ is CFH₂. |
| 562B | Q¹ is 5-Cl-3-pyridazinyl, R² is Br and R⁴ is Me. |
| 563B | Q¹ is 5-Cl-3-pyridazinyl, R² is I and R⁴ is Me. |
| 564B | Q¹ is 5-Cl-3-pyridazinyl, R² is Me and R⁴ is Me. |
| 565B | Q¹ is 5-Cl-3-pyridazinyl, R² is Me and R⁴ is Cl. |
| 566B | Q¹ is 5-Cl-3-pyridazinyl, R² is Me and R⁴ is Br. |
| 567B | Q¹ is 5-Cl-3-pyridazinyl, R² is Me and R⁴ is I. |
| 568B | Q¹ is 5-Cl-3-pyridazinyl, R² is Br and R⁴ is Br. |
| 569B | Q¹ is 5-Cl-3-pyridazinyl, R² is Br and R⁴ is Cl. |
| 570B | Q¹ is 5-Cl-3-pyridazinyl, R² is Cl and R⁴ is Br. |
| 571B | Q¹ is 5-Cl-3-pyridazinyl, R² is Cl and R⁴ is Cl. |
| 572B | Q¹ is 5-Cl-3-pyridazinyl, R² is Me and R⁴ is H. |
| 573B | Q¹ is 5-Cl-3-pyridazinyl, R² is Cl and R⁴ is H. |
| 574B | Q¹ is 5-Cl-3-pyridazinyl, R² is Br and R⁴ is H. |
| 575B | Q¹ is 5-F-3-pyridazinyl, R² is Cl and R⁴ is Me. |
| 576B | Q¹ is 5-F-3-pyridazinyl, R² is Cl and R⁴ is CFH₂. |
| 577B | Q¹ is 5-F-3-pyridazinyl, R² is Br and R⁴ is Me. |
| 578B | Q¹ is 5-F-3-pyridazinyl, R² is I and R⁴ is Me. |
| 579B | Q¹ is 5-F-3-pyridazinyl, R² is Me and R⁴ is Me. |
| 580B | Q¹ is 5-F-3-pyridazinyl, R² is Me and R⁴ is Cl. |
| 581B | Q¹ is 5-F-3-pyridazinyl, R² is Me and R⁴ is Br. |
| 582B | Q¹ is 5-F-3-pyridazinyl, R² is Me and R⁴ is I. |
| 583B | Q¹ is 5-F-3-pyridazinyl, R² is Br and R⁴ is Br. |
| 584B | Q¹ is 5-F-3-pyridazinyl, R² is Br and R⁴ is Cl. |
| 585B | Q¹ is 5-F-3-pyridazinyl, R² is Cl and R⁴ is Br. |
| 586B | Q¹ is 5-F-3-pyridazinyl, R² is Cl and R⁴ is Cl. |
| 587B | Q¹ is 5-F-3-pyridazinyl, R² is Me and R⁴ is H. |
| 588B | Q¹ is 5-F-3-pyridazinyl, R² is Cl and R⁴ is H. |
| 589B | Q¹ is 5-F-3-pyridazinyl, R² is Br and R⁴ is H. |
| 590B | Q¹ is 5-MeO-3-pyridazinyl, R² is Cl and R⁴ is Me. |
| 591B | Q¹ is 5-MeO-3-pyridazinyl, R² is Cl and R⁴ is CFH₂. |
| 592B | Q¹ is 5-MeO-3-pyridazinyl, R² is Br and R⁴ is Me. |
| 593B | Q¹ is 5-MeO-3-pyridazinyl, R² is I and R⁴ is Me. |
| 594B | Q¹ is 5-MeO-3-pyridazinyl, R² is Me and R⁴ is Me. |
| 595B | Q¹ is 5-MeO-3-pyridazinyl, R² is Me and R⁴ is Cl. |
| 596B | Q¹ is 5-MeO-3-pyridazinyl, R² is Me and R⁴ is Br. |
| 597B | Q¹ is 5-MeO-3-pyridazinyl, R² is Me and R⁴ is I. |
| 598B | Q¹ is 5-MeO-3-pyridazinyl, R² is Br and R⁴ is Br. |
| 599B | Q¹ is 5-MeO-3-pyridazinyl, R² is Br and R⁴ is Cl. |
| 600B | Q¹ is 5-MeO-3-pyridazinyl, R² is Cl and R⁴ is Br. |
| 601B | Q¹ is 5-MeO-3-pyridazinyl, R² is Cl and R⁴ is Cl. |
| 602B | Q¹ is 5-MeO-3-pyridazinyl, R² is Me and R⁴ is H. |
| 603B | Q¹ is 5-MeO-3-pyridazinyl, R² is Cl and R⁴ is H. |
| 604B | Q¹ is 5-MeO-3-pyridazinyl, R² is Br and R⁴ is H. |
| 605B | Q¹ is 2-Cl-5-pyrimidinyl, R² is Cl and R⁴ is Me. |
| 606B | Q¹ is 2-Cl-5-pyrimidinyl, R² is Cl and R⁴ is CFH₂. |
| 607B | Q¹ is 2-Cl-5-pyrimidinyl, R² is Br and R⁴ is Me. |
| 608B | Q¹ is 2-Cl-5-pyrimidinyl, R² is I and R⁴ is Me. |
| 609B | Q¹ is 2-Cl-5-pyrimidinyl, R² is Me and R⁴ is Me. |
| 610B | Q¹ is 2-Cl-5 pyrimidinyl, R² is Me and R⁴ is Cl. |
| 611B | Q¹ is 2-Cl-5-pyrimidinyl, R² is Me and R⁴ is Br. |
| 612B | Q¹ is 2-Cl-5-pyrimidinyl, R² is Me and R⁴ is I. |
| 613B | Q¹ is 2-Cl-5-pyrimidinyl, R² is Br and R⁴ is Br. |
| 614B | Q¹ is 2-Cl-5-pyrimidinyl, R² is Br and R⁴ is Cl. |
| 615B | Q¹ is 2-Cl-5-pyrimidinyl, R² is Cl and R⁴ is Br. |
| 616B | Q¹ is 2-Cl-5-pyrimidinyl, R² is Cl and R⁴ is Cl. |
| 617B | Q¹ is 2-Cl-5-pyrimidinyl, R² is Me and R⁴ is H. |
| 618B | Q¹ is 2-Cl-5-pyrimidinyl, R² is Cl and R⁴ is H. |
| 619B | Q¹ is 2-Cl-5-pyrimidinyl, R² is Br and R⁴ is H. |
| 620B | Q¹ is 2-Me-5-pyrimidinyl, R² is Cl and R⁴ is Me. |
| 621B | Q¹ is 2-Me-5-pyrimidinyl, R² is Cl and R⁴ is CFH₂. |
| 622B | Q¹ is 2-Me-5-pyrimidinyl, R² is Br and R⁴ is Me. |
| 623B | Q¹ is 2-Me-5-pyrimidinyl, R² is I and R⁴ is Me. |
| 624B | Q¹ is 2-Me-5-pyrimidinyl, R² is Me and R⁴ is Me. |
| 625B | Q¹ is 2-Me-5-pyrimidinyl, R² is Me and R⁴ is Cl. |
| 626B | Q¹ is 2-Me-5-pyrimidinyl, R² is Me and R⁴ is Br. |
| 627B | Q¹ is 2-Me-5-pyrimidinyl, R² is Me and R⁴ is I. |
| 628B | Q¹ is 2-Me-5-pyrimidinyl, R² is Br and R⁴ is Br. |
| 629B | Q¹ is 2-Me-5-pyrimidinyl, R² is Br and R⁴ is Cl. |
| 630B | Q¹ is 2-Me-5-pyrimidinyl, R² is Cl and R⁴ is Br. |
| 631B | Q¹ is 2-Me-5-pyrimidinyl, R² is Cl and R⁴ is Cl. |
| 632B | Q¹ is 2-Me-5-pyrimidinyl, R² is Me and R⁴ is H. |
| 633B | Q¹ is 2-Me-5-pyrimidinyl, R² is Cl and R⁴ is H. |
| 634B | Q¹ is 2-Me-5-pyrimidinyl, R² is Br and R⁴ is H. |
| 635B | Q¹ is 2-MeO-5-pyrimidinyl, R² is Cl and R⁴ is Me. |
| 636B | Q¹ is 2-MeO-5-pyrimidinyl, R² is Cl and R⁴ is CFH₂. |
| 637B | Q¹ is 2-MeO-5-pyrimidinyl, R² is Br and R⁴ is Me. |
| 638B | Q¹ is 2-MeO-5-pyrimidinyl, R² is I and R⁴ is Me. |
| 639B | Q¹ is 2-MeO-5-pyrimidinyl, R² is Me and R⁴ is Me. |
| 640B | Q¹ is 2-MeO-5-pyrimidinyl, R² is Me and R⁴ is Cl. |
| 641B | Q¹ is 2-MeO-5-pyrimidinyl, R² is Me and R⁴ is Br. |
| 642B | Q¹ is 2-MeO-5-pyrimidinyl, R² is Me and R⁴ is I. |
| 643B | Q¹ is 2-MeO-5 pyrimidinyl, R² is Br and R⁴ is Br. |
| 644B | Q¹ is 2-MeO-5-pyrimidinyl, R² is Br and R⁴ is Cl. |
| 645B | Q¹ is 2-MeO-5-pyrimidinyl, R² is Cl and R⁴ is Br. |
| 646B | Q¹ is 2-MeO-5-pyrimidinyl, R² is Cl and R⁴ is Cl. |
| 647B | Q¹ is 2-MeO-5-pyrimidinyl, R² is Me and R⁴ is H. |
| 648B | Q¹ is 2-MeO-5-pyrimidinyl, R² is Cl and R⁴ is H. |
| 649B | Q¹ is 2-MeO-5-pyrimidinyl, R² is Br and R⁴ is H. |
| 650B | Q¹ is 2-CF₃-5-pyrimidinyl, R² is Cl and R⁴ is Me. |
| 651B | Q¹ is 2-CF₃-5-pyrimidinyl, R² is Cl and R⁴ is CFH₂. |
| 652B | Q¹ is 2-CF₃-5-pyrimidinyl, R² is Br and R⁴ is Me. |
| 653B | Q¹ is 2-CF₃-5-pyrimidinyl, R² is I and R⁴ is Me. |
| 654B | Q¹ is 2-CF₃-5-pyrimidinyl, R² is Me and R⁴ is Me. |
| 655B | Q¹ is 2-CF₃-5-pyrimidinyl, R² is Me and R⁴ is Cl. |
| 656B | Q¹ is 2-CF₃-5-pyrimidinyl, R² is Me and R⁴ is Br. |
| 657B | Q¹ is 2-CF₃-5-pyrimidinyl, R² is Me and R⁴ is I. |
| 658B | Q¹ is 2-CF₃-5-pyrimidinyl, R² is Br and R⁴ is Br. |
| 659B | Q¹ is 2-CF₃-5-pyrimidinyl, R² is Br and R⁴ is Cl. |
| 660B | Q¹ is 2-CF₃-5-pyrimidinyl, R² is Cl and R⁴ is Br. |
| 661B | Q¹ is 2-CF₃-5-pyrimidinyl, R² is Cl and R⁴ is Cl. |
| 662B | Q¹ is 2-CF₃-5-pyrimidinyl, R² is Me and R⁴ is H. |
| 663B | Q¹ is 2-CF₃-5-pyrimidinyl, R² is Cl and R⁴ is H. |
| 664B | Q¹ is 2-CF₃-5-pyrimidinyl, R² is Br and R⁴ is H. |
| 665B | Q¹ is 5-Cl-2-pyrimidinyl, R² is Cl and R⁴ is Me. |
| 666B | Q¹ is 5-Cl-2-pyrimidinyl, R² is Cl and R⁴ is CFH₂. |
| 667B | Q¹ is 5-Cl-2-pyrimidinyl, R² is Br and R⁴ is Me. |
| 668B | Q¹ is 5-Cl-2-pyrimidinyl, R² is I and R⁴ is Me. |
| 669B | Q¹ is 5-Cl-2-pyrimidinyl, R² is Me and R⁴ is Me. |
| 670B | Q¹ is 5-Cl-2-pyrimidinyl, R² is Me and R⁴ is Cl. |
| 671B | Q¹ is 5-Cl-2-pyrimidinyl, R² is Me and R⁴ is Br. |
| 672B | Q¹ is 5-Cl-2-pyrimidinyl, R² is Me and R⁴ is I. |
| 673B | Q¹ is 5-Cl-2-pyrimidinyl, R² is Br and R⁴ is Br. |
| 674B | Q¹ is 5-Cl-2-pyrimidinyl, R² is Br and R⁴ is Cl. |
| 675B | Q¹ is 5-Cl-2-pyrimidinyl, R² is Cl and R⁴ is Br. |
| 676B | Q¹ is 5-Cl-2-pyrimidinyl, R² is Cl and R⁴ is Cl. |
| 677B | Q¹ is 5-Cl-2-pyrimidinyl, R² is Me and R⁴ is H. |
| 678B | Q¹ is 5-Cl-2-pyrimidinyl, R² is Cl and R⁴ is H. |
| 679B | Q¹ is 5-Cl-2-pyrimidinyl, R² is Br and R⁴ is H. |
| 680B | Q¹ is 5-Me-2-pyrimidinyl, R² is Cl and R⁴ is Me. |
| 681B | Q¹ is 5-Me-2-pyrimidinyl, R² is Cl and R⁴ is CFH₂. |
| 682B | Q¹ is 5-Me-2-pyrimidinyl, R² is Br and R⁴ is Me. |
| 683B | Q¹ is 5-Me-2-pyrimidinyl, R² is I and R⁴ is Me. |
| 684B | Q¹ is 5-Me-2-pyrimidinyl, R² is Me and R⁴ is Me. |
| 685B | Q¹ is 5-Me-2-pyrimidinyl, R² is Me and R⁴ is Cl. |
| 686B | Q¹ is 5-Me-2-pyrimidinyl, R² is Me and R⁴ is Br. |
| 687B | Q¹ is 5-Me-2-pyrimidinyl, R² is Me and R⁴ is I. |
| 688B | Q¹ is 5-Me-2-pyrimidinyl, R² is Br and R⁴ is Br. |
| 689B | Q¹ is 5-Me-2-pyrimidinyl, R² is Br and R⁴ is Cl. |
| 690B | Q¹ is 5-Me-2-pyrimidinyl, R² is Cl and R⁴ is Br. |
| 691B | Q¹ is 5-Me-2-pyrimidinyl, R² is Cl and R⁴ is Cl. |
| 692B | Q¹ is 5-Me-2-pyrimidinyl, R² is Me and R⁴ is H. |
| 693B | Q¹ is 5-Me-2-pyrimidinyl, R² is Cl and R⁴ is H. |
| 694B | Q¹ is 5-Me-2-pyrimidinyl, R² is Br and R⁴ is H. |
| 695B | Q¹ is 5-MeO-2-pyrimidinyl, R² is Cl and R⁴ is Me. |
| 696B | Q¹ is 5-MeO-2-pyrimidinyl, R² is Cl and R⁴ is CFH₂. |
| 697B | Q¹ is 5-MeO-2-pyrimidinyl, R² is Br and R⁴ is Me. |
| 698B | Q¹ is 5-MeO-2-pyrimidinyl, R² is I and R⁴ is Me. |
| 699B | Q¹ is 5-MeO-2-pyrimidinyl, R² is Me and R⁴ is Me. |
| 700B | Q¹ is 5-MeO-2-pyrimidinyl, R² is Me and R⁴ is Cl. |
| 701B | Q¹ is 5-MeO-2-pyrimidinyl, R² is Me and R⁴ is Br. |
| 702B | Q¹ is 5-MeO-2-pyrimidinyl, R² is Me and R⁴ is I. |
| 703B | Q¹ is 5-MeO-2-pyrimidinyl, R² is Br and R⁴ is Br. |
| 704B | Q¹ is 5-MeO-2-pyrimidinyl, R² is Br and R⁴ is Cl. |
| 705B | Q¹ is 5-MeO-2-pyrimidinyl, R² is Cl and R⁴ is Br. |
| 706B | Q¹ is 5-MeO-2-pyrimidinyl, R² is Cl and R⁴ is Cl. |
| 707B | Q¹ is 5-MeO-2-pyrimidinyl, R² is Me and R⁴ is H. |
| 708B | Q¹ is 5-MeO-2-pyrimidinyl, R² is Cl and R⁴ is H. |
| 709B | Q¹ is 5-MeO-2-pyrimidinyl, R² is Br and R⁴ is H. |
| 710B | Q¹ is 5-Cl-2-pyrimidinyl, R² is Cl and R⁴ is Me. |
| 711B | Q¹ is 5-Cl-2-pyrimidinyl, R² is Cl and R⁴ is CFH₂. |
| 712B | Q¹ is 5-Cl-2-pyrimidinyl, R² is Br and R⁴ is Me. |
| 713B | Q¹ is 5-Cl-2-pyrimidinyl, R² is I and R⁴ is Me. |
| 714B | Q¹ is 5-Cl-2-pyrimidinyl, R² is Me and R⁴ is Me. |
| 715B | Q¹ is 5-Cl-2-pyrimidinyl, R² is Me and R⁴ is Cl. |
| 716B | Q¹ is 5-Cl-2-pyrimidinyl, R² is Me and R⁴ is Br. |
| 717B | Q¹ is 5-Cl-2-pyrimidinyl, R² is Me and R⁴ is I. |
| 718B | Q¹ is 5-Cl-2-pyrimidinyl, R² is Br and R⁴ is Br. |
| 719B | Q¹ is 5-Cl-2-pyrimidinyl, R² is Br and R⁴ is Cl. |
| 720B | Q¹ is 5-Cl-2-pyrimidinyl, R² is Cl and R⁴ is Br. |
| 721B | Q¹ is 5-Cl-2-pyrimidinyl, R² is Cl and R⁴ is Cl. |
| 722B | Q¹ is 5-Cl-2-pyrimidinyl, R² is Me and R⁴ is H. |
| 723B | Q¹ is 5-Cl-2-pyrimidinyl, R² is Cl and R⁴ is H. |
| 724B | Q¹ is 5-Cl-2-pyrimidinyl, R² is Br and R⁴ is H. |
| 725B | Q¹ is 5-CF₃-2-pyrimidinyl, R² is Cl and R⁴ is Me. |
| 726B | Q¹ is 5-CF₃-2-pyrimidinyl, R² is Cl and R⁴ is CFH₂. |
| 727B | Q¹ is 5-CF₃-2-pyrimidinyl, R² is Br and R⁴ is Me. |
| 728B | Q¹ is 5-CF₃-2-pyrimidinyl, R² is I and R⁴ is Me. |
| 729B | Q¹ is 5-CF₃-2-pyrimidinyl, R² is Me and R⁴ is Me. |
| 730B | Q¹ is 5-CF₃-2-pyrimidinyl, R² is Me and R⁴ is Cl. |
| 731B | Q¹ is 5-CF₃-2-pyrimidinyl, R² is Me and R⁴ is Br. |
| 732B | Q¹ is 5-CF₃-2-pyrimidinyl, R² is Me and R⁴ is I. |
| 733B | Q¹ is 5-CF₃-2-pyrimidinyl, R² is Br and R⁴ is Br. |
| 734B | Q¹ is 5-CF₃-2-pyrimidinyl, R² is Br and R⁴ is Cl. |
| 735B | Q¹ is 5-CF₃-2-pyrimidinyl, R² is Cl and R⁴ is Br. |
| 736B | Q¹ is 5-CF₃-2-pyrimidinyl, R² is Cl and R⁴ is Cl. |
| 737B | Q¹ is 5-CF₃-2-pyrimidinyl, R² is Me and R⁴ is H. |
| 738B | Q¹ is 5-CF₃-2-pyrimidinyl, R² is Cl and R⁴ is H. |
| 739B | Q¹ is 5-CF₃-2-pyrimidinyl, R² is Br and R⁴ is H. |
| 740B | Q¹ is 4,6-di-MeO-1,3,4-triazin-2-yl, R² is Cl and R⁴ is Me. |
| 741B | Q¹ is 4,6-di-MeO-1,3,4-triazin-2-yl, R² is Cl and R⁴ is CFH₂. |
| 742B | Q¹ is 4,6-di-MeO-1,3,4-triazin-2-yl, R² is Br and R⁴ is Me. |
| 743B | Q¹ is 4,6-di-MeO-1,3,4-triazin-2-yl, R² is I and R⁴ is Me. |
| 744B | Q¹ is 4,6-di-MeO-1,3,4-triazin-2-yl, R² is Me and R⁴ is Me. |
| 745B | Q¹ is 4,6-di-MeO-1,3,4-triazin-2-yl, R² is Me and R⁴ is Cl. |
| 746B | Q¹ is 4,6-di-MeO-1,3,4-triazin-2-yl, R² is Me and R⁴ is Br. |
| 747B | Q¹ is 4,6-di-MeO-1,3,4-triazin-2-yl, R² is Me and R⁴ is I. |
| 748B | Q¹ is 4,6-di-MeO-1,3,4-triazin-2-yl, R² is Br and R⁴ is Br. |
| 749B | Q¹ is 4,6-di-MeO-1,3,4-triazin-2-yl, R² is Br and R⁴ is Cl. |
| 750B | Q¹ is 4,6-di-MeO-1,3,4-triazin-2-yl, R² is Cl and R⁴ is Br. |
| 751B | Q¹ is 4,6-di-MeO-1,3,4-triazin-2-yl, R² is Cl and R⁴ is Cl. |
| 752B | Q¹ is 4,6-di-MeO-1,3,4-triazin-2-yl, R² is Me and R⁴ is H. |
| 753B | Q¹ is 4,6-di-MeO-1,3,4-triazin-2-yl, R² is Cl and R⁴ is H. |
| 754B | Q¹ is 4,6-di-MeO-1,3,4-triazin-2-yl, R² is Br and R⁴ is H. |
| 755B | Q¹ is 5-Me-2-thienyl, R² is Cl and R⁴ is Me. |
| 756B | Q¹ is 5-Me-2-thienyl, R² is Cl and R⁴ is CFH₂. |
| 757B | Q¹ is 5-Me-2-thienyl, R² is Br and R⁴ is Me. |
| 758B | Q¹ is 5-Me-2-thienyl, R² is I and R⁴ is Me. |
| 759B | Q¹ is 5-Me-2-thienyl, R² is Me and R⁴ is Me. |
| 760B | Q¹ is 5-Me-2-thienyl, R² is Me and R⁴ is Cl. |
| 761B | Q¹ is 5-Me-2-thienyl, R² is Me and R⁴ is Br. |
| 762B | Q¹ is 5-Me-2-thienyl, R² is Me and R⁴ is I. |
| 763B | Q¹ is 5-Me-2-thienyl, R² is Br and R⁴ is Br. |
| 764B | Q¹ is 5-Me-2-thienyl, R² is Br and R⁴ is Cl. |
| 765B | Q¹ is 5-Me-2-thienyl, R² is Cl and R⁴ is Br. |
| 766B | Q¹ is 5-Me-2-thienyl, R² is Cl and R⁴ is Cl. |
| 767B | Q¹ is 5-Me-2-thienyl, R² is Me and R⁴ is H. |
| 768B | Q¹ is 5-Me-2-thienyl, R² is Cl and R⁴ is H. |
| 769B | Q¹ is 5-Me-2-thienyl, R² is Br and R⁴ is H. |
| 770B | Q¹ is 5-Cl-2-thienyl, R² is Cl and R⁴ is Me. |
| 771B | Q¹ is 5-Cl-2-thienyl, R² is Cl and R⁴ is CFH₂. |
| 772B | Q¹ is 5-Cl-2-thienyl, R² is Br and R⁴ is Me. |
| 773B | Q¹ is 5-Cl-2-thienyl, R² is I and R⁴ is Me. |
| 774B | Q¹ is 5-Cl-2-thienyl, R² is Me and R⁴ is Me. |
| 775B | Q¹ is 5-Cl-2-thienyl, R² is Me and R⁴ is Cl. |
| 776B | Q¹ is 5-Cl-2-thienyl, R² is Me and R⁴ is Br. |
| 777B | Q¹ is 5-Cl-2-thienyl, R² is Me and R⁴ is I. |
| 778B | Q¹ is 5-Cl-2-thienyl, R² is Br and R⁴ is Br. |
| 779B | Q¹ is 5-Cl-2-thienyl, R² is Br and R⁴ is Cl. |
| 780B | Q¹ is 5-Cl-2-thienyl, R² is Cl and R⁴ is Br. |
| 781B | Q¹ is 5-Cl-2-thienyl, R² is Cl and R⁴ is Cl. |
| 782B | Q¹ is 5-Cl-2-thienyl, R² is Me and R⁴ is H. |
| 783B | Q¹ is 5-Cl-2-thienyl, R² is Cl and R⁴ is H. |
| 784B | Q¹ is 5-Cl-2-thienyl, R² is Br and R⁴ is H. |
| 785B | Q¹ is 5-F-2-thienyl, R² is Cl and R⁴ is Me. |
| 786B | Q¹ is 5-F-2-thienyl, R² is Cl and R⁴ is CFH₂. |
| 787B | Q¹ is 5-F-2-thienyl, R² is Br and R⁴ is Me. |
| 788B | Q¹ is 5-F-2-thienyl, R² is I and R⁴ is Me. |
| 789B | Q¹ is 5-F-2-thienyl, R² is Me and R⁴ is Me. |
| 790B | Q¹ is 5-F-2-thienyl, R² is Me and R⁴ is Cl. |
| 791B | Q¹ is 5-F-2-thienyl, R² is Me and R⁴ is Br. |
| 792B | Q¹ is 5-F-2-thienyl, R² is Me and R⁴ is I. |
| 793B | Q¹ is 5-F-2-thienyl, R² is Br and R⁴ is Br. |
| 794B | Q¹ is 5-F-2-thienyl, R² is Br and R⁴ is Cl. |
| 795B | Q¹ is 5-F-2-thienyl, R² is Cl and R⁴ is Br. |
| 796B | Q¹ is 5-F-2-thienyl, R² is Cl and R⁴ is Cl. |
| 797B | Q¹ is 5-F-2-thienyl, R² is Me and R⁴ is H. |
| 798B | Q¹ is 5-F-2-thienyl, R² is Cl and R⁴ is H. |
| 799B | Q¹ is 5-F-2-thienyl, R² is Br and R⁴ is H. |
| 800B | Q¹ is 5-Me-3-thienyl, R² is Cl and R⁴ is Me. |
| 801B | Q¹ is 5-Me-3-thienyl, R² is Cl and R⁴ is CFH₂. |
| 802B | Q¹ is 5-Me-3-thienyl, R² is Br and R⁴ is Me. |
| 803B | Q¹ is 5-Me-3-thienyl, R² is I and R⁴ is Me. |
| 804B | Q¹ is 5-Me-3-thienyl, R² is Me and R⁴ is Me. |
| 805B | Q¹ is 5-Me-3-thienyl, R² is Me and R⁴ is Cl. |
| 806B | Q¹ is 5-Me-3-thienyl, R² is Me and R⁴ is Br. |
| 807B | Q¹ is 5-Me-3-thienyl, R² is Me and R⁴ is I. |
| 808B | Q¹ is 5-Me-3-thienyl, R² is Br and R⁴ is Br. |
| 809B | Q¹ is 5-Me-3-thienyl, R² is Br and R⁴ is Cl. |
| 810B | Q¹ is 5-Me-3-thienyl, R² is Cl and R⁴ is Br. |
| 811B | Q¹ is 5-Me-3-thienyl, R² is Cl and R⁴ is Cl. |
| 812B | Q¹ is 5-Me-3-thienyl, R² is Me and R⁴ is H. |
| 813B | Q¹ is 5-Me-3-thienyl, R² is Cl and R⁴ is H. |
| 814B | Q¹ is 5-Me-3-thienyl, R² is Br and R⁴ is H. |
| 815B | Q¹ is 5-Cl-3-thienyl, R² is Cl and R⁴ is Me. |
| 816B | Q¹ is 5-Cl-3-thienyl, R² is Cl and R⁴ is CFH₂. |
| 817B | Q¹ is 5-Cl-3-thienyl, R² is Br and R⁴ is Me. |
| 818B | Q¹ is 5-Cl-3-thienyl, R² is I and R⁴ is Me. |
| 819B | Q¹ is 5-Cl-3-thienyl, R² is Me and R⁴ is Me. |
| 820B | Q¹ is 5-Cl-3-thienyl, R² is Me and R⁴ is Cl. |
| 821B | Q¹ is 5-Cl-3-thienyl, R² is Me and R⁴ is Br. |
| 822B | Q¹ is 5-Cl-3-thienyl, R² is Me and R⁴ is I. |
| 823B | Q¹ is 5-Cl-3-thienyl, R² is Br and R⁴ is Br. |
| 824B | Q¹ is 5-Cl-3-thienyl, R² is Br and R⁴ is Cl. |
| 825B | Q¹ is 5-Cl-3-thienyl, R² is Cl and R⁴ is Br. |
| 826B | Q¹ is 5-Cl-3-thienyl, R² is Cl and R⁴ is Cl. |
| 827B | Q¹ is 5-Cl-3-thienyl, R² is Me and R⁴ is H. |
| 828B | Q¹ is 5-Cl-3-thienyl, R² is Cl and R⁴ is H. |
| 829B | Q¹ is 5-Cl-3-thienyl, R² is Br and R⁴ is H. |
| 830B | Q¹ is 5-F-3-thienyl, R² is Cl and R⁴ is Me. |
| 831B | Q¹ is 5-F-3-thienyl, R² is Cl and R⁴ is CFH₂. |
| 832B | Q¹ is 5-F-3-thienyl, R² is Br and R⁴ is Me. |
| 833B | Q¹ is 5-F-3-thienyl, R² is I and R⁴ is Me. |
| 834B | Q¹ is 5-F-3-thienyl, R² is Me and R⁴ is Me. |
| 835B | Q¹ is 5-F-3-thienyl, R² is Me and R⁴ is Cl. |
| 836B | Q¹ is 5-F-3-thienyl, R² is Me and R⁴ is Br. |
| 837B | Q¹ is 5-F-3-thienyl, R² is Me and R⁴ is I. |
| 838B | Q¹ is 5-F-3-thienyl, R² is Br and R⁴ is Br. |
| 839B | Q¹ is 5-F-3-thienyl, R² is Br and R⁴ is Cl. |
| 840B | Q¹ is 5-F-3-thienyl, R² is Cl and R⁴ is Br. |
| 841B | Q¹ is 5-F-3-thienyl, R² is Cl and R⁴ is Cl. |
| 842B | Q¹ is 5-F-3-thienyl, R² is Me and R⁴ is H. |
| 843B | Q¹ is 5-F-3-thienyl, R² is Cl and R⁴ is H. |
| 844B | Q¹ is 5-F-3-thienyl, R² is Br and R⁴ is H. |
| 845B | Q¹ is 1-Me-1H-pyrazol-3-yl, R² is Cl and R⁴ is Me. |
| 846B | Q¹ is 1-Me-1H-pyrazol-3-yl, R² is Cl and R⁴ is CFH₂. |
| 847B | Q¹ is 1-Me-1H-pyrazol-3-yl, R² is Br and R⁴ is Me. |
| 848B | Q¹ is 1-Me-1H-pyrazol-3-yl, R² is I and R⁴ is Me. |
| 849B | Q¹ is 1-Me-1H-pyrazol-3-yl, R² is Me and R⁴ is Me. |
| 850B | Q¹ is 1-Me-1H-pyrazol-3-yl, R² is Me and R⁴ is Cl. |
| 851B | Q¹ is 1-Me-1H-pyrazol-3-yl, R² is Me and R⁴ is Br. |
| 852B | Q¹ is 1-Me-1H-pyrazol-3-yl, R² is Me and R⁴ is I. |
| 853B | Q¹ is 1-Me-1H-pyrazol-3-yl, R² is Br and R⁴ is Br. |
| 854B | Q¹ is 1-Me-1H-pyrazol-3-yl, R² is Br and R⁴ is Cl. |
| 855B | Q¹ is 1-Me-1H-pyrazol-3-yl, R² is Cl and R⁴ is Br. |
| 856B | Q¹ is 1-Me-1H-pyrazol-3-yl, R² is Cl and R⁴ is Cl. |
| 857B | Q¹ is 1-Me-1H-pyrazol-3-yl, R² is Me and R⁴ is H. |
| 858B | Q¹ is 1-Me-1H-pyrazol-3-yl, R² is Cl and R⁴ is H. |
| 859B | Q¹ is 1-Me-1H-pyrazol-3-yl, R² is Br and R⁴ is H. |
| 860B | Q¹ is 1-Me-1H-pyrazol-4-yl, R² is Cl and R⁴ is Me. |
| 861B | Q¹ is 1-Me-1H-pyrazol-4-yl, R² is Cl and R⁴ is CFH₂. |
| 862B | Q¹ is 1-Me-1H-pyrazol-4-yl, R² is Br and R⁴ is Me. |
| 863B | Q¹ is 1-Me-1H-pyrazol-4-yl, R² is I and R⁴ is Me. |
| 864B | Q¹ is 1-Me-1H-pyrazol-4-yl, R² is Me and R⁴ is Me. |
| 865B | Q¹ is 1-Me-1H-pyrazol-4-yl, R² is Me and R⁴ is Cl. |
| 866B | Q¹ is 1-Me-1H-pyrazol-4-yl, R² is Me and R⁴ is Br. |
| 867B | Q¹ is 1-Me-1H-pyrazol-4-yl, R² is Me and R⁴ is I. |
| 868B | Q¹ is 1-Me-1H-pyrazol-4-yl, R² is Br and R⁴ is Br. |
| 869B | Q¹ is 1-Me-1H-pyrazol-4-yl, R² is Br and R⁴ is Cl. |
| 870B | Q¹ is 1-Me-1H-pyrazol-4-yl, R² is Cl and R⁴ is Br. |
| 871B | Q¹ is 1-Me-1H-pyrazol-4-yl, R² is Cl and R⁴ is Cl. |
| 872B | Q¹ is 1-Me-1H-pyrazol-4-yl, R² is Me and R⁴ is H. |
| 873B | Q¹ is 1-Me-1H-pyrazol-4-yl, R² is Cl and R⁴ is H. |
| 874B | Q¹ is 1-Me-1H-pyrazol-4-yl, R² is Br and R⁴ is H. |
| 875B | Q¹ is 2-Me-5-thiazolyl, R² is Cl and R⁴ is Me. |
| 876B | Q¹ is 2-Me-5-thiazolyl, R² is Cl and R⁴ is CFH₂. |
| 877B | Q¹ is 2-Me-5-thiazolyl, R² is Br and R⁴ is Me. |
| 878B | Q¹ is 2-Me-5-thiazolyl, R² is I and R⁴ is Me. |
| 879B | Q¹ is 2-Me-5-thiazolyl, R² is Me and R⁴ is Me. |
| 880B | Q¹ is 2-Me-5-thiazolyl, R² is Me and R⁴ is Cl. |
| 881B | Q¹ is 2-Me-5-thiazolyl, R² is Me and R⁴ is Br. |
| 882B | Q¹ is 2-Me-5-thiazolyl, R² is Me and R⁴ is I. |
| 883B | Q¹ is 2-Me-5-thiazolyl, R² is Br and R⁴ is Br. |
| 884B | Q¹ is 2-Me-5-thiazolyl, R² is Br and R⁴ is Cl. |
| 885B | Q¹ is 2-Me-5-thiazolyl, R² is Cl and R⁴ is Br. |
| 886B | Q¹ is 2-Me-5-thiazolyl, R² is Cl and R⁴ is Cl. |
| 887B | Q¹ is 2-Me-5-thiazolyl, R² is Me and R⁴ is H. |
| 888B | Q¹ is 2-Me-5-thiazolyl, R² is Cl and R⁴ is H. |
| 889B | Q¹ is 2-Me-5-thiazolyl, R² is Br and R⁴ is H. |
| 890B | Q¹ is 2-Cl-5-thiazolyl, R² is Cl and R⁴ is Me. |
| 891B | Q¹ is 2-Cl-5-thiazolyl, R² is Cl and R⁴ is CFH₂. |
| 892B | Q¹ is 2-Cl-5-thiazolyl, R² is Br and R⁴ is Me. |
| 893B | Q¹ is 2-Cl-5-thiazolyl, R² is I and R⁴ is Me. |
| 894B | Q¹ is 2-Cl-5-thiazolyl, R² is Me and R⁴ is Me. |
| 895B | Q¹ is 2-Cl-5-thiazolyl, R² is Me and R⁴ is Cl. |
| 896B | Q¹ is 2-Cl-5-thiazolyl, R² is Me and R⁴ is Br. |
| 897B | Q¹ is 2-Cl-5-thiazolyl, R² is Me and R⁴ is I. |
| 898B | Q¹ is 2-Cl-5-thiazolyl, R² is Br and R⁴ is Br. |
| 899B | Q¹ is 2-Cl-5-thiazolyl, R² is Br and R⁴ is Cl. |
| 900B | Q¹ is 2-Cl-5-thiazolyl, R² is Cl and R⁴ is Br. |
| 901B | Q¹ is 2-Cl-5-thiazolyl, R² is Cl and R⁴ is Cl. |
| 902B | Q¹ is 2-Cl-5-thiazolyl, R² is Me and R⁴ is H. |
| 903B | Q¹ is 2-Cl-5-thiazolyl, R² is Cl and R⁴ is H. |
| 904B | Q¹ is 2-Cl-5-thiazolyl, R² is Br and R⁴ is H. |
| 905B | Q¹ is 5-Me-3-isothiazolyl, R² is Cl and R⁴ is Me. |
| 906B | Q¹ is 5-Me-3-isothiazolyl, R² is Cl and R⁴ is CFH₂. |
| 907B | Q¹ is 5-Me-3-isothiazolyl, R² is Br and R⁴ is Me. |
| 908B | Q¹ is 5-Me-3-isothiazolyl, R² is I and R⁴ is Me. |
| 909B | Q¹ is 5-Me-3-isothiazolyl, R² is Me and R⁴ is Me. |
| 910B | Q¹ is 5-Me-3-isothiazolyl, R² is Me and R⁴ is Cl. |
| 911B | Q¹ is 5-Me-3-isothiazolyl, R² is Me and R⁴ is Br. |
| 912B | Q¹ is 5-Me-3-isothiazolyl, R² is Me and R⁴ is I. |
| 913B | Q¹ is 5-Me-3-isothiazolyl, R² is Br and R⁴ is Br. |
| 914B | Q¹ is 5-Me-3-isothiazolyl, R² is Br and R⁴ is Cl. |
| 915B | Q¹ is 5-Me-3-isothiazolyl, R² is Cl and R⁴ is Br. |
| 916B | Q¹ is 5-Me-3-isothiazolyl, R² is Cl and R⁴ is Cl. |
| 917B | Q¹ is 5-Me-3-isothiazolyl, R² is Me and R⁴ is H. |
| 918B | Q¹ is 5-Me-3-isothiazolyl, R² is Cl and R⁴ is H. |
| 919B | Q¹ is 5-Me-3-isothiazolyl, R² is Br and R⁴ is H. |
| 920B | Q¹ is 5-Cl-3-isothiazolyl, R² is Cl and R⁴ is Me. |
| 921B | Q¹ is 5-Cl-3-isothiazolyl, R² is Cl and R⁴ is CFH₂. |
| 922B | Q¹ is 5-Cl-3-isothiazolyl, R² is Br and R⁴ is Me. |
| 923B | Q¹ is 5-Cl-3-isothiazolyl, R² is I and R⁴ is Me. |
| 924B | Q¹ is 5-Cl-3-isothiazolyl, R² is Me and R⁴ is Me. |
| 925B | Q¹ is 5-Cl-3-isothiazolyl, R² is Me and R⁴ is Cl. |
| 926B | Q¹ is 5-Cl-3-isothiazolyl, R² is Me and R⁴ is Br. |
| 927B | Q¹ is 5-Cl-3-isothiazolyl, R² is Me and R⁴ is I. |
| 928B | Q¹ is 5-Cl-3-isothiazolyl, R² is Br and R⁴ is Br. |
| 929B | Q¹ is 5-Cl-3-isothiazolyl, R² is Br and R⁴ is Cl. |
| 930B | Q¹ is 5-Cl-3-isothiazolyl, R² is Cl and R⁴ is Br. |
| 931B | Q¹ is 5-Cl-3-isothiazolyl, R² is Cl and R⁴ is Cl. |
| 932B | Q¹ is 5-Cl-3-isothiazolyl, R² is Me and R⁴ is H. |
| 933B | Q¹ is 5-Cl-3-isothiazolyl, R² is Cl and R⁴ is H. |
| 934B | Q¹ is 5-Cl-3-isothiazolyl, R² is Br and R⁴ is H. |

TABLE 3

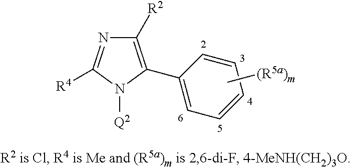

| Q² | Q² | Q² | Q² | Q² |
|---|---|---|---|---|
| 4-Cl—Ph | 3-F—Ph | 3,4-di-F—Ph | 6-CF₃-3-pyridinyl | 5-Me-3-pyridinyl |
| 4-F—Ph | 3-Br—Ph | 3,4-di-Cl—Ph | 2-Cl, 6-CF₃-3-pyridinyl | 5-F-3-pyridinyl |
| 4-Br—Ph | 3-Me—Ph | 2-Cl, 3,5-di-MeO—Ph | 2-Cl, 6-MeO-3-pyridinyl | 6-Me-3-pyridinyl |
| 4-Me—Ph | 3-Et—Ph | 4-Cl, 3,5-di-MeO—Ph | 2-Cl, 6-Me-3-pyridinyl | 6-Cl-3-pyridinyl |
| 4-Et—Ph | 3-F, 4-Me—Ph | 5-MeO-3-pyridinyl | 6-MeO-3-pyridinyl | 5-Cl-3-pyridinyl |
| 4-Cl—Bn | 4-Cl, 3-F—Ph | 6-MeO-3-pyridinyl | 6-Br-3-pyridinyl | 6-CF₃-3-pyridinyl |
| 4-F—Bn | 2-Cl, 4-F—Ph | 2-Me-5-pyridinyl | 2-CF₃-5-pyridinyl | 2-Cl-5-pyridinyl |
| 3-Cl—Ph | 3,5-di-MeO—Ph | 6-F-3-pyridinyl | 6-Me-3-pyridinyl | 6-Cl-3-pyridinyl |
| | | | | 2-MeO-5-pyridinyl |

The present disclosure also includes Tables 1C through 62C, each of which is constructed the same as Table 3 above except that the row heading in Table 3 (i.e. “R² is Cl, R⁴ is Me and (R^{5a})ₘ is 2,6-di-F, 4-MeNH(CH₂)₃O”) is replaced with the respective row heading shown below. Thus, for example, in Table 1C the row heading is “R² is Cl, R⁴ is Cl and (R^{5a})ₘ is 2,6-di-F, 4-MeNH(CH₂)₃O”, and Q² is as defined in Table 3 above. Tables 2C through 62C are constructed similarly.

| Table | |
|---|---|
| Number | Row Heading |
| 1C | R² is Cl, R⁴ is Cl and (R^{5a})ₘ is 2,6-di-F, 4-MeNH(CH₂)₃O. |
| 2C | R² is Cl, R⁴ is Br and (R^{5a})ₘ is 2,6-di-F, 4-MeNH(CH₂)₃O. |
| 3C | R² is Br, R⁴ is Me and (R^{5a})ₘ is 2,6-di-F, 4-MeNH(CH₂)₃O. |
| 4C | R² is Br, R⁴ is Cl and (R^{5a})ₘ is 2,6-di-F, 4-MeNH(CH₂)₃O. |
| 5C | R² is Me, R⁴ is Me and (R^{5a})ₘ is 2,6-di-F, 4-MeNH(CH₂)₃O. |
| 6C | R² is Br, R⁴ is Br and (R^{5a})ₘ is 2,6-di-F, 4-MeNH(CH₂)₃O. |
| 7C | R² is Cl, R⁴ is Me and (R^{5a})ₘ is 2,6-di-F, 4-Me₂N(CH₂)₃O. |
| 8C | R² is Cl, R⁴ is Cl and (R^{5a})ₘ is 2,6-di-F, 4-Me₂N(CH₂)₃O. |
| 9C | R² is Cl, R⁴ is Br and (R^{5a})ₘ is 2,6-di-F, 4-Me₂N(CH₂)₃O. |
| 10C | R² is Br, R⁴ is Me and (R^{5a})ₘ is 2,6-di-F, 4-Me₂N(CH₂)₃O. |
| 11C | R² is Br, R⁴ is Cl and (R^{5a})ₘ is 2,6-di-F, 4-Me₂N(CH₂)₃O. |
| 12C | R² is Me, R⁴ is Me and (R^{5a})ₘ is 2,6-di-F, 4-Me₂N(CH₂)₃O. |
| 13C | R² is Br, R⁴ is Br and (R^{5a})ₘ is 2,6-di-F, 4-Me₂N(CH₂)₃O. |
| 14C | R² is Cl, R⁴ is Me, and (R^{5a})ₘ is 2,6-di-F, 4-MeO(CH₂)₃O. |
| 15C | R² is Cl, R⁴ is Cl and (R^{5a})ₘ is 2,6-di-F, 4-MeO(CH₂)₃O. |
| 16C | R² is Cl, R⁴ is Br and (R^{5a})ₘ is 2,6-di-F, 4-MeO(CH₂)₃O. |
| 17C | R² is Br, R⁴ is Me and (R^{5a})ₘ is 2,6-di-F, 4-MeO(CH₂)₃O. |
| 18C | R² is Br, R⁴ is Cl and (R^{5a})ₘ is 2,6-di-F, 4-MeO(CH₂)₃O. |
| 19C | R² is Me, R⁴ is Me and (R^{5a})ₘ is 2,6-di-F, 4-MeO(CH₂)₃O. |
| 20C | R² is Br, R⁴ is Br and (R^{5a})ₘ is 2,6-di-F, 4-MeO(CH₂)₃O. |
| 21C | R² is Cl, R⁴ is Me and (R^{5a})ₘ is 2-Cl-6-F, 4-MeNH(CH₂)₃O. |
| 22C | R² is Cl, R⁴ is Cl and (R^{5a})ₘ is 2-Cl-6-F, 4-MeNH(CH₂)₃O. |
| 23C | R² is Cl, R⁴ is Br and (R^{5a})ₘ is 2-Cl-6-F, 4-MeNH(CH₂)₃O. |
| 24C | R² is Br, R⁴ is Me and (R^{5a})ₘ is 2-Cl-6-F, 4-MeNH(CH₂)₃O. |
| 25C | R² is Br, R⁴ is Cl and (R^{5a})ₘ is 2-Cl-6-F, 4-MeNH(CH₂)₃O. |
| 26C | R² is Me, R⁴ is Me and (R^{5a})ₘ is 2-Cl-6-F, 4-MeNH(CH₂)₃O. |
| 27C | R² is Br, R⁴ is Br and (R^{5a})ₘ is 2-Cl-6-F, 4-MeNH(CH₂)₃O. |
| 28C | R² is Cl, R⁴ is Me and (R^{5a})ₘ is 2,6-di-F, 3-MeNH(CH₂)₃O. |
| 29C | R² is Cl, R⁴ is Cl and (R^{5a})ₘ is 2,6-di-F, 3-MeNH(CH₂)₃O. |
| 30C | R² is Cl, R⁴ is Br and (R^{5a})ₘ is 2,6-di-F, 3-MeNH(CH₂)₃O. |
| 31C | R² is Br, R⁴ is Me and (R^{5a})ₘ is 2,6-di-F, 3-MeNH(CH₂)₃O. |
| 32C | R² is Br, R⁴ is Cl and (R^{5a})ₘ is 2,6-di-F, 3-MeNH(CH₂)₃O. |
| 33C | R² is Me, R⁴ is Me and (R^{5a})ₘ is 2,6-di-F, 3-MeNH(CH₂)₃O. |
| 34C | R² is Br, R⁴ is Br and (R^{5a})ₘ is 2,6-di-F, 3-MeNH(CH₂)₃O. |
| 35C | R² is Cl, R⁴ is Me and (R^{5a})ₘ is 2,6-di-F, 3-Me₂N(CH₂)₃O. |
| 36C | R² is Cl, R⁴ is Cl and (R^{5a})ₘ is 2,6-di-F, 3-Me₂N(CH₂)₃O. |
| 37C | R² is Cl, R⁴ is Br and (R^{5a})ₘ is 2,6-di-F, 3-Me₂N(CH₂)₃O. |
| 38C | R² is Br, R⁴ is Me and (R^{5a})ₘ is 2,6-di-F, 3-Me₂N(CH₂)₃O. |
| 39C | R² is Br, R⁴ is Cl and (R^{5a})ₘ is 2,6-di-F, 3-Me₂N(CH₂)₃O. |
| 40C | R² is Me, R⁴ is Me and (R^{5a})ₘ is 2,6-di-F, 3-Me₂N(CH₂)₃O. |
| 41C | R² is Br, R⁴ is Br and (R^{5a})ₘ is 2,6-di-F, 3-Me₂N(CH₂)₃O. |
| 42C | R² is Cl, R⁴ is Me, and (R^{5a})ₘ is 2,6-di-F, 3-MeO(CH₂)₃O. |
| 43C | R² is Cl, R⁴ is Cl and (R^{5a})ₘ is 2,6-di-F, 3-MeO(CH₂)₃O. |
| 44C | R² is Cl, R⁴ is Br and (R^{5a})ₘ is 2,6-di-F, 3-MeO(CH₂)₃O. |
| 45C | R² is Br, R⁴ is Me and (R^{5a})ₘ is 2,6-di-F, 3-MeO(CH₂)₃O. |
| 46C | R² is Br, R⁴ is Cl and (R^{5a})ₘ is 2,6-di-F, 3-MeO(CH₂)₃O. |
| 46C | R² is Me, R⁴ is Me and (R^{5a})ₘ is 2,6-di-F, 3-MeO(CH₂)₃O. |
| 48C | R² is Br, R⁴ is Br and (R^{5a})ₘ is 2,6-di-F, 3-MeO(CH₂)₃O. |
| 49C | R² is Cl, R⁴ is Me and (R^{5a})ₘ is 2-Cl-6-F, 3-MeNH(CH₂)₃O. |
| 50C | R² is Cl, R⁴ is Cl and (R^{5a})ₘ is 2-Cl-6-F, 3-MeNH(CH₂)₃O. |
| 51C | R² is Cl, R⁴ is Br and (R^{5a})ₘ is 2-Cl-6-F, 3-MeNH(CH₂)₃O. |
| 52C | R² is Br, R⁴ is Me and (R^{5a})ₘ is 2-Cl-6-F, 3-MeNH(CH₂)₃O. |
| 53C | R² is Br, R⁴ is Cl and (R^{5a})ₘ is 2-Cl-6-F, 3-MeNH(CH₂)₃O. |
| 54C | R² is Me, R⁴ is Me and (R^{5a})ₘ is 6-Cl-6-F, 3-MeNH(CH₂)₃O. |
| 55C | R² is Br, R⁴ is Br and (R^{5a})ₘ is 2-Cl-6-F, 3-MeNH(CH₂)₃O. |
| 56C | R² is Cl, R⁴ is Me and (R^{5a})ₘ is 6-Cl-2-F, 3-MeNH(CH₂)₃O. |
| 57C | R² is Cl, R⁴ is Cl and (R^{5a})ₘ is 6-Cl-2-F, 3-MeNH(CH₂)₃O. |
| 58C | R² is Cl, R⁴ is Br and (R^{5a})ₘ is 6-Cl-2-F, 3-MeNH(CH₂)₃O. |
| 59C | R² is Br, R⁴ is Me and (R^{5a})ₘ is 6-Cl-2-F, 3-MeNH(CH₂)₃O. |
| 60C | R² is Br, R⁴ is Cl and (R^{5a})ₘ is 6-Cl-2-F, 3-MeNH(CH₂)₃O. |
| 61C | R² is Me, R⁴ is Me and (R^{5a})ₘ is 6-Cl-2-F, 3-MeNH(CH₂)₃O. |
| 62C | R² is Br, R⁴ is Br and (R^{5a})ₘ is 6-Cl-2-F, 3-MeNH(CH₂)₃O. |

TABLE 4

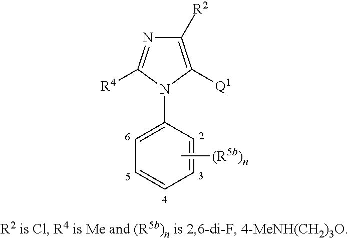

| Q¹ | Q¹ | Q¹ | Q¹ | Q¹ |
|---|---|---|---|---|
| 4-Cl—Ph | 3-F—Ph | 3,4-di-F—Ph | 6-CF₃-3-pyridinyl | 5-Me-3-pyridinyl |
| 4-F—Ph | 3-Br—Ph | 3,4-di-Cl—Ph | 2-Cl, 6-CF₃-3-pyridinyl | 5-F-3-pyridinyl |
| 4-Br—Ph | 3-Me—Ph | 2-Cl, 3,5-di-MeO—Ph | 2-Cl, 6-MeO-3-pyridinyl | 6-Me-3-pyridinyl |
| 4-Me—Ph | 3-Et—Ph | 4-Cl, 3,5-di-MeO—Ph | 2-Cl, 6-Me-3-pyridinyl | 6-Cl-3-pyridinyl |
| 4-Et—Ph | 3-F, 4-Me—Ph | 5-MeO-3-pyridinyl | 6-MeO-3-pyridinyl | 5-Cl-3-pyridinyl |
| 4-Cl—Bn | 4-Cl, 3-F—Ph | 6-MeO-3-pyridinyl | 6-Br-3-pyridinyl | 6-CF₃-3-pyridinyl |
| 4-F—Bn | 2-Cl, 4-F—Ph | 2-Me-5-pyridinyl | 2-CF₃-5-pyridinyl | 2-Cl-5-pyridinyl |
| 3-Cl—Ph | 3,5-di-MeO—Ph | 6-F-3-pyridinyl | 6-Me-3-pyridinyl | 6-Cl-3-pyridinyl |
| | | | | 2-MeO-5-pyridinyl |

The present disclosure also includes Tables 1D through 62D, each of which is constructed the same as Table 4 above except that the row heading in Table 4 (i.e. “R² is Cl, R⁴ is Me and (R^{5b})ₙ is 2,6-di-F, 4-MeNH(CH₂)₃O”) is replaced with the respective row heading shown below. Thus, for example, in Table 1D the row heading is “R² is Cl, R⁴ is Cl and (R^{5b})ₙ is 2,6-di-F, 4-MeNH(CH₂)₃O”, and Q¹ is as defined in Table 4 above. Tables 2D through 62D are constructed similarly.

| Table | |
|---|---|
| Number | Row Heading |
| 1D | R² is Cl, R⁴ is Cl and (R^{5b})ₙ is 2,6-di-F, 4-MeNH(CH₂)₃O. |
| 2D | R² is Cl, R⁴ is Br and (R^{5b})ₙ is 2,6-di-F, 4-MeNH(CH₂)₃O. |
| 3D | R² is Br, R⁴ is Me and (R^{5b})ₙ is 2,6-di-F, 4-MeNH(CH₂)₃O. |
| 4D | R² is Br, R⁴ is Cl and (R^{5b})ₙ is 2,6-di-F, 4-MeNH(CH₂)₃O. |
| 5D | R² is Me, R⁴ is Me and (R^{5b})ₙ is 2,6-di-F, 4-MeNH(CH₂)₃O. |
| 6D | R² is Br, R⁴ is Br and (R^{5b})ₙ is 2,6-di-F, 4-MeNH(CH₂)₃O. |
| 7D | R² is Cl, R⁴ is Me and (R^{5b})ₙ is 2,6-di-F, 4-Me₂N(CH₂)₃O. |
| 8D | R² is Cl, R⁴ is Cl and (R^{5b})ₙ is 2,6-di-F, 4-Me₂N(CH₂)₃O. |
| 9D | R² is Cl, R⁴ is Br and (R^{5b})ₙ is 2,6-di-F, 4-Me₂N(CH₂)₃O. |
| 10D | R² is Br, R⁴ is Me and (R^{5b})ₙ is 2,6-di-F, 4-Me₂N(CH₂)₃O. |
| 11D | R² is Br, R⁴ is Cl and (R^{5b})ₙ is 2,6-di-F, 4-Me₂N(CH₂)₃O. |
| 12D | R² is Me, R⁴ is Me and (R^{5b})ₙ is 2,6-di-F, 4-Me₂N(CH₂)₃O. |
| 13D | R² is Br, R⁴ is Br and (R^{5b})ₙ is 2,6-di-F, 4-Me₂N(CH₂)₃O. |
| 14D | R² is Cl, R⁴ is Me, and (R^{5b})ₙ is 2,6-di-F, 4-MeO(CH₂)₃O. |
| 15D | R² is Cl, R⁴ is Cl and (R^{5b})ₙ is 2,6-di-F, 4-MeO(CH₂)₃O. |
| 16D | R² is Cl, R⁴ is Br and (R^{5b})ₙ is 2,6-di-F, 4-MeO(CH₂)₃O. |
| 17D | R² is Br, R⁴ is Me and (R^{5b})ₙ is 2,6-di-F, 4-MeO(CH₂)₃O. |
| 18D | R² is Br, R⁴ is Cl and (R^{5b})ₙ is 2,6-di-F, 4-MeO(CH₂)₃O. |
| 19D | R² is Me, R⁴ is Me and (R^{5b})ₙ is 2,6-di-F, 4-MeO(CH₂)₃O. |
| 20D | R² is Br, R⁴ is Br and (R^{5b})ₙ is 2,6-di-F, 4-MeO(CH₂)₃O. |
| 21D | R² is Cl, R⁴ is Me and (R^{5b})ₙ is 2-Cl-6-F, 4-MeNH(CH₂)₃O. |
| 22D | R² is Cl, R⁴ is Cl and (R^{5b})ₙ is 2-Cl-6-F, 4-MeNH(CH₂)₃O. |
| 23D | R² is Cl, R⁴ is Br and (R^{5b})ₙ is 2-Cl-6-F, 4-MeNH(CH₂)₃O. |
| 24D | R² is Br, R⁴ is Me and (R^{5b})ₙ is 2-Cl-6-F, 4-MeNH(CH₂)₃O. |
| 25D | R² is Br, R⁴ is Cl and (R^{5b})ₙ is 2-Cl-6-F, 4-MeNH(CH₂)₃O. |
| 26D | R² is Me, R⁴ is Me and (R^{5b})ₙ is 2-Cl-6-F, 4-MeNH(CH₂)₃O. |
| 27D | R² is Br, R⁴ is Br and (R^{5b})ₙ is 2-Cl-6-F, 4-MeNH(CH₂)₃O. |
| 28D | R² is Cl, R⁴ is Me and (R^{5b})ₙ is 2,6-di-F, 3-MeNH(CH₂)₃O. |
| 29D | R² is Cl, R⁴ is Cl and (R^{5b})ₙ is 2,6-di-F, 3-MeNH(CH₂)₃O. |
| 30D | R² is Cl, R⁴ is Br and (R^{5b})ₙ is 2,6-di-F, 3-MeNH(CH₂)₃O. |
| 31D | R² is Br, R⁴ is Me and (R^{5b})ₙ is 2,6-di-F, 3-MeNH(CH₂)₃O. |
| 32D | R² is Br, R⁴ is Cl and (R^{5b})ₙ is 2,6-di-F, 3-MeNH(CH₂)₃O. |
| 33D | R² is Me, R⁴ is Me and (R^{5b})ₙ is 2,6-di-F, 3-MeNH(CH₂)₃O. |
| 34D | R² is Br, R⁴ is Br and (R^{5b})ₙ is 2,6-di-F, 3-MeNH(CH₂)₃O. |
| 35D | R² is Cl, R⁴ is Me and (R^{5b})ₙ is 2,6-di-F, 3-Me₂N(CH₂)₃O. |
| 36D | R² is Cl, R⁴ is Cl and (R^{5b})ₙ is 2,6-di-F, 3-Me₂N(CH₂)₃O. |
| 37D | R² is Cl, R⁴ is Br and (R^{5b})ₙ is 2,6-di-F, 3-Me₂N(CH₂)₃O. |
| 38D | R² is Br, R⁴ is Me and (R^{5b})ₙ is 2,6-di-F, 3-Me₂N(CH₂)₃O. |
| 39D | R² is Br, R⁴ is Cl and (R^{5b})ₙ is 2,6-di-F, 3-Me₂N(CH₂)₃O. |
| 40D | R² is Me, R⁴ is Me and (R^{5b})ₙ is 2,6-di-F, 3-Me₂N(CH₂)₃O. |
| 41D | R² is Br, R⁴ is Br and (R^{5b})ₙ is 2,6-di-F, 3-Me₂N(CH₂)₃O. |
| 42D | R² is Cl, R⁴ is Me, and (R^{5b})ₙ is 2,6-di-F, 3-MeO(CH₂)₃O. |
| 43D | R² is Cl, R⁴ is Cl and (R^{5b})ₙ is 2,6-di-F, 3-MeO(CH₂)₃O. |
| 44D | R² is Cl, R⁴ is Br and (R^{5b})ₙ is 2,6-di-F, 3-MeO(CH₂)₃O. |
| 45D | R² is Br, R⁴ is Me and (R^{5b})ₙ is 2,6-di-F, 3-MeO(CH₂)₃O. |
| 46D | R² is Br, R⁴ is Cl and (R^{5b})ₙ is 2,6-di-F, 3-MeO(CH₂)₃O. |
| 46D | R² is Me, R⁴ is Me and (R^{5b})ₙ is 2,6-di-F, 3-MeO(CH₂)₃O. |
| 48D | R² is Br, R⁴ is Br and (R^{5b})ₙ is 2,6-di-F, 3-MeO(CH₂)₃O. |
| 49D | R² is Cl, R⁴ is Me and (R^{5b})ₙ is 2-Cl-6-F, 3-MeNH(CH₂)₃O. |
| 50D | R² is Cl, R⁴ is Cl and (R^{5b})ₙ is 2-Cl-6-F, 3-MeNH(CH₂)₃O. |
| 51D | R² is Cl, R⁴ is Br and (R^{5b})ₙ is 2-Cl-6-F, 3-MeNH(CH₂)₃O. |
| 52D | R² is Br, R⁴ is Me and (R^{5b})ₙ is 2-Cl-6-F, 3-MeNH(CH₂)₃O. |
| 53D | R² is Br, R⁴ is Cl and (R^{5b})ₙ is 2-Cl-6-F, 3-MeNH(CH₂)₃O. |
| 54D | R² is Me, R⁴ is Me and (R^{5b})ₙ is 6-Cl-6-F, 3-MeNH(CH₂)₃O. |
| 55D | R² is Br, R⁴ is Br and (R^{5b})ₙ is 2-Cl-6-F, 3-MeNH(CH₂)₃O. |
| 56D | R² is Cl, R⁴ is Me and (R^{5b})ₙ is 6-Cl-2-F, 3-MeNH(CH₂)₃O. |
| 57D | R² is Cl, R⁴ is Cl and (R^{5b})ₙ is 6-Cl-2-F, 3-MeNH(CH₂)₃O. |
| 58D | R² is Cl, R⁴ is Br and (R^{5b})ₙ is 6-Cl-2-F, 3-MeNH(CH₂)₃O. |
| 59D | R² is Br, R⁴ is Me and (R^{5b})ₙ is 6-Cl-2-F, 3-MeNH(CH₂)₃O. |
| 60D | R² is Br, R⁴ is Cl and (R^{5b})ₙ is 6-Cl-2-F, 3-MeNH(CH₂)₃O. |
| 61D | R² is Me, R⁴ is Me and (R^{5b})ₙ is 6-Cl-2-F, 3-MeNH(CH₂)₃O. |
| 62D | R² is Br, R⁴ is Br and (R^{5b})ₙ is 6-Cl-2-F, 3-MeNH(CH₂)₃O. |

TABLE 5

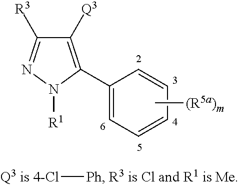

| (R^{5a})ₘ | (R^{5a})ₘ | (R^{5a})ₘ | (R^{5a})ₘ | (R^{5a})ₘ |
|---|---|---|---|---|
| 2,6-di-F | 2,4-di-Cl | 4-CN, 2,6-di-F | 2-CF₃, 4-F | 2-Cl, 4-NO₂ |
| 2,4,6-tri-F | 2-Cl, 4,6-di-F | 2,6-di-F, 4-Me | 2-CF₂HO, 4-F | 2-NO₂, 4-F |
| 2,3,6-tri-F | 2-Cl, 6-F | 2-Cl, 5-CF₃ | 2-CN, 6-F | 2,3,4,5,6-penta-F |
| 2,4,5-tri-F | 2-Br, 6-F | 2-Cl, 4-Me | 2,5-di-Cl | 2,5-di-Cl, 4-F |
| 2,3,4-tri-F | 2-F, 6-CF₃ | 2-Cl, 4-MeO | 2-CF₃, 4-MeO | 2,3-di-Cl, 4-F |
| 2-Cl, 4-F | 2-F, 6-CF₂HO | 2-Br, 4-MeO | 2-F, 6-Me | 2-Cl, 5-CN |
| 2-Br, 4-F | 2-I, 4-F | 2,6-di-F, 3-Cl | 2,6-di-F, 3-Me | 2,4-di-F, 5-CN |
| 2,4-di-F | 4-Cl, 2,6-di-F | 2,6-di-F, 3-CN | 2-CF₃ | 2-Cl, 6-F, 3-MeO |
| 2,6-di-Cl | 2,6-di-F, 4-MeO | 2,6-di-F, 3-MeO | 2-CF₃O | |
| 2,6-di-F, 4-EtO | 2-Cl, 6-F, 4-MeO | 2,6-di-F, 4-CF₂HO | 2,6-di-F, 4-NO₂ | |
| 2-Cl, 3,6-di-F | 2-Cl, 6-F, 5-MeO | 2,6-di-F, 3-CF₂HO | 2,6-di-F, 3-EtO | |

The present disclosure also includes Tables 1E through 387E, each of which is constructed the same as Table 5 above except that the row heading in Table 5 (i.e. “Q³ is 4-Cl-Ph, R³ is Cl and R¹ is Me”) is replaced with the respective row heading shown below. Thus, for example, in Table 1E the row heading is “Q³ is 4-Cl-Ph, R³ is Cl and R¹ is CFH₂”, and (R^{5a})ₘ is as defined in Table 5 above. Tables 2E through 387E are constructed similarly.

| Table Number | Row Heading |
|---|---|
| 1E | Q³ is 4-Cl-Ph, R³ is Cl and R¹ is CFH₂. |
| 2E | Q³ is 4-Cl-Ph, R³ is Br and R¹ is Me. |
| 3E | Q³ is 4-Cl-Ph, R³ is I and R¹ is Me. |
| 4E | Q³ is 4-Cl-Ph, R³ is Me and R¹ is Me. |
| 5E | Q³ is 4-Cl-Ph, R³ is MeO and R¹ is Me. |
| 6E | Q³ is 4-Cl-Ph, R³ is MeS and R¹ is Me. |
| 7E | Q³ is 4-Cl-Ph, R³ is Et and R¹ is Me. |
| 8E | Q³ is 4-Cl-Ph, R³ is Me and R¹ is Et. |
| 9E | Q³ is 4-Cl-Ph, R³ is Cl and R¹ is Et. |
| 10E | Q³ is 3-Cl-Ph, R³ is Cl and R¹ is Me. |
| 11E | Q³ is 3-Cl-Ph, R³ is Cl and R¹ is CFH₂. |
| 12E | Q³ is 3-Cl-Ph, R³ is Br and R¹ is Me. |
| 13E | Q³ is 3-Cl-Ph, R³ is I and R¹ is Me. |
| 14E | Q³ is 3-Cl-Ph, R³ is Me and R¹ is Me. |
| 15E | Q³ is 3-Cl-Ph, R³ is MeO and R¹ is Me. |
| 16E | Q³ is 3-Cl-Ph, R³ is MeS and R¹ is Me. |
| 17E | Q³ is 3-Cl-Ph, R³ is Et and R¹ is Me. |
| 18E | Q³ is 3-Cl-Ph, R³ is Me and R¹ is Et. |
| 19E | Q³ is 3-Cl-Ph, R³ is Cl and R¹ is Et. |
| 20E | Q³ is 4-F-Ph, R³ is Cl and R¹ is Me. |
| 21E | Q³ is 4-F-Ph, R³ is Cl and R¹ is CFH₂. |
| 22E | Q³ is 4-F-Ph, R³ is Br and R¹ is Me. |
| 23E | Q³ is 4-F-Ph, R³ is I and R¹ is Me. |
| 24E | Q³ is 4-F-Ph, R³ is Me and R¹ is Me. |
| 25E | Q³ is 4-F-Ph, R³ is MeO and R¹ is Me. |
| 26E | Q³ is 4-F-Ph, R³ is MeS and R¹ is Me. |
| 27E | Q³ is 4-F-Ph, R³ is Et and R¹ is Me. |
| 28E | Q³ is 4-F-Ph, R³ is Me and R¹ is Et. |
| 29E | Q³ is 4-F-Ph, R³ is Cl and R¹ is Et. |
| 30E | Q³ is 3-F-Ph, R³ is Cl and R¹ is Me. |
| 31E | Q³ is 3-F-Ph, R³ is Cl and R¹ is CFH₂. |
| 32E | Q³ is 3-F-Ph, R³ is Br and R¹ is Me. |
| 33E | Q³ is 3-F-Ph, R³ is I and R¹ is Me. |
| 34E | Q³ is 3-F-Ph, R³ is Me and R¹ is Me. |
| 35E | Q³ is 3-F-Ph, R³ is MeO and R¹ is Me. |
| 36E | Q³ is 3-F-Ph, R³ is MeS and R¹ is Me. |
| 37E | Q³ is 3-F-Ph, R³ is Et and R¹ is Me. |
| 38E | Q³ is 3-F-Ph, R³ is Me and R¹ is Et. |
| 39E | Q³ is 3-F-Ph, R³ is Cl and R¹ is Et. |
| 40E | Q³ is 3-CF₂HO-Ph, R³ is Cl and R¹ is Me. |
| 41E | Q³ is 3-CF₂HO-Ph, R³ is Cl and R¹ is CFH₂. |
| 42E | Q³ is 3-CF₂HO-Ph, R³ is Br and R¹ is Me. |
| 43E | Q³ is 3-CF₂HO-Ph, R³ is I and R¹ is Me. |
| 44E | Q³ is 3-CF₂HO-Ph, R³ is Me and R¹ is Me. |
| 45E | Q³ is 3-CF₂HO-Ph, R³ is MeO and R¹ is Me. |
| 46E | Q³ is 3-CF₂HO-Ph, R³ is MeS and R¹ is Me. |
| 47E | Q³ is 3-CF₂HO-Ph, R³ is Et and R¹ is Me. |
| 48E | Q³ is 3-CF₂HO-Ph, R³ is Me and R¹ is Et. |
| 49E | Q³ is 3-CF₂HO-Ph, R³ is Cl and R¹ is Et. |
| 50E | Q³ is 4-Me-Ph, R³ is Cl and R¹ is Me. |
| 51E | Q³ is 4-Me-Ph, R³ is Cl and R¹ is CFH₂. |
| 52E | Q³ is 4-Me-Ph, R³ is Br and R¹ is Me. |
| 53E | Q³ is 4-Me-Ph, R³ is I and R¹ is Me. |
| 54E | Q³ is 4-Me-Ph, R³ is Me and R¹ is Me. |
| 55E | Q³ is 4-Me-Ph, R³ is MeO and R¹ is Me. |
| 56E | Q³ is 4-Me-Ph, R³ is MeS and R¹ is Me. |
| 57E | Q³ is 4-Me-Ph, R³ is Et and R¹ is Me. |
| 58E | Q³ is 4-Me-Ph, R³ is Me and R¹ is Et. |
| 59E | Q³ is 4-Me-Ph, R³ is Cl and R¹ is Et. |
| 60E | Q³ is 3-Me-Ph, R³ is Cl and R¹ is Me. |
| 61E | Q³ is 3-Me-Ph, R³ is Cl and R¹ is CFH₂. |
| 62E | Q³ is 3-Me-Ph, R³ is Br and R¹ is Me. |
| 63E | Q³ is 3-Me-Ph, R³ is I and R¹ is Me. |
| 64E | Q³ is 3-Me-Ph, R³ is Me and R¹ is Me. |
| 65E | Q³ is 3-Me-Ph, R³ is MeO and R¹ is Me. |
| 66E | Q³ is 3-Me-Ph, R³ is MeS and R¹ is Me. |
| 67E | Q³ is 3-Me-Ph, R³ is Et and R¹ is Me. |
| 68E | Q³ is 3-Me-Ph, R³ is Me and R¹ is Et. |
| 69E | Q³ is 3-Me-Ph, R³ is Cl and R¹ is Et. |
| 70E | Q³ is 4-Et-Ph, R³ is Cl and R¹ is Me. |
| 71E | Q³ is 4-Et-Ph, R³ is Cl and R¹ is CFH₂. |
| 72E | Q³ is 4-Et-Ph, R³ is Br and R¹ is Me. |
| 73E | Q³ is 4-Et-Ph, R³ is I and R¹ is Me. |
| 74E | Q³ is 4-Et-Ph, R³ is Me and R¹ is Me. |
| 75E | Q³ is 4-Et-Ph, R³ is MeO and R¹ is Me. |
| 76E | Q³ is 4-Et-Ph, R³ is MeS and R¹ is Me. |
| 77E | Q³ is 4-Et-Ph, R³ is Et and R¹ is Me. |
| 78E | Q³ is 4-Et-Ph, R³ is Me and R¹ is Et. |
| 79E | Q³ is 4-Et-Ph, R³ is Cl and R¹ is Et. |
| 80E | Q³ is 4-Cl, 3-F-Ph, R³ is Cl and R¹ is Me. |
| 81E | Q³ is 4-Cl, 3-F-Ph, R³ is Cl and R¹ is CFH₂. |
| 82E | Q³ is 4-Cl, 3-F-Ph, R³ is Br and R¹ is Me. |
| 83E | Q³ is 4-Cl, 3-F-Ph, R³ is I and R¹ is Me. |
| 84E | Q³ is 4-Cl, 3-F-Ph, R³ is Me and R¹ is Me. |
| 85E | Q³ is 4-Cl, 3-F-Ph, R³ is MeO and R¹ is Me. |
| 86E | Q³ is 4-Cl, 3-F-Ph, R³ is MeS and R¹ is Me. |
| 87E | Q³ is 4-Cl, 3-F-Ph, R³ is Et and R¹ is Me. |
| 88E | Q³ is 4-Cl, 3-F-Ph, R³ is Me and R¹ is Et. |
| 89E | Q³ is 4-Cl, 3-F-Ph, R³ is Cl and R¹ is Et. |
| 90E | Q³ is 2-Cl, 4-F-Ph, R³ is Cl and R¹ is Me. |
| 91E | Q³ is 2-Cl, 4-F-Ph, R³ is Cl and R¹ is CFH₂. |
| 92E | Q³ is 2-Cl, 4-F-Ph, R³ is Br and R¹ is Me. |
| 93E | Q³ is 2-Cl, 4-F-Ph, R³ is I and R¹ is Me. |
| 94E | Q³ is 2-Cl, 4-F-Ph, R³ is Me and R¹ is Me. |
| 95E | Q³ is 2-Cl, 4-F-Ph, R³ is MeO and R¹ is Me. |
| 96E | Q³ is 2-Cl, 4-F-Ph, R³ is MeS and R¹ is Me. |
| 97E | Q³ is 2-Cl, 4-F-Ph, R³ is Et and R¹ is Me. |
| 98E | Q³ is 2-Cl, 4-F-Ph, R³ is Me and R¹ is Et. |
| 99E | Q³ is 2-Cl, 4-F-Ph, R³ is Cl and R¹ is Et. |
| 100E | Q³ is 4-F, 3-Me-Ph, R³ is Cl and R¹ is Me. |
| 101E | Q³ is 4-F, 3-Me-Ph, R³ is Cl and R¹ is CFH₂. |
| 102E | Q³ is 4-F, 3-Me-Ph, R³ is Br and R¹ is Me. |
| 103E | Q³ is 4-F, 3-Me-Ph, R³ is I and R¹ is Me. |
| 104E | Q³ is 4-F, 3-Me-Ph, R³ is Me and R¹ is Me. |
| 105E | Q³ is 4-F, 3-Me-Ph, R³ is MeO and R¹ is Me. |
| 106E | Q³ is 4-F, 3-Me-Ph, R³ is MeS and R¹ is Me. |
| 107E | Q³ is 4-F, 3-Me-Ph, R³ is Et and R¹ is Me. |
| 108E | Q³ is 4-F, 3-Me-Ph, R³ is Me and R¹ is Et. |
| 109E | Q³ is 4-F, 3-Me-Ph, R³ is Cl and R¹ is Et. |
| 110E | Q³ is 3,4-di-F-Ph, R³ is Cl and R¹ is Me. |
| 111E | Q³ is 3,4-di-F-Ph, R³ is Cl and R¹ is CFH₂. |
| 112E | Q³ is 3,4-di-F-Ph, R³ is Br and R¹ is Me. |
| 113E | Q³ is 3,4-di-F-Ph, R³ is I and R¹ is Me. |
| 114E | Q³ is 3,4-di-F-Ph, R³ is Me and R¹ is Me. |
| 115E | Q³ is 3,4-di-F-Ph, R³ is MeO and R¹ is Me. |
| 116E | Q³ is 3,4-di-F-Ph, R³ is MeS and R¹ is Me. |
| 117E | Q³ is 3,4-di-F-Ph, R³ is Et and R¹ is Me. |
| 118E | Q³ is 3,4-di-F-Ph, R³ is Me and R¹ is Et. |
| 119E | Q³ is 3,4-di-F-Ph, R³ is Cl and R¹ is Et. |
| 120E | Q³ is 3,4-di-Cl-Ph, R³ is Cl and R¹ is Me. |
| 121E | Q³ is 3,4-di-Cl-Ph, R³ is Cl and R¹ is CFH₂. |
| 122E | Q³ is 3,4-di-Cl-Ph, R³ is Br and R¹ is Me. |
| 123E | Q³ is 3,4-di-Cl-Ph, R³ is I and R¹ is Me. |
| 124E | Q³ is 3,4-di-Cl-Ph, R³ is Me and R¹ is Me. |
| 125E | Q³ is 3,4-di-Cl-Ph, R³ is MeO and R¹ is Me. |
| 126E | Q³ is 3,4-di-Cl-Ph, R³ is MeS and R¹ is Me. |
| 127E | Q³ is 3,4-di-Cl-Ph, R³ is Et and R¹ is Me. |
| 128E | Q³ is 3,4-di-Cl-Ph, R³ is Me and R¹ is Et. |
| 129E | Q³ is 3,4-di-Cl-Ph, R³ is Cl and R¹ is Et. |
| 130E | Q³ is 3,5-di-MeO-Ph, R³ is Cl and R¹ is Me. |
| 131E | Q³ is 3,5-di-MeO-Ph, R³ is Cl and R¹ is CFH₂. |
| 132E | Q³ is 3,5-di-MeO-Ph, R³ is Br and R¹ is Me. |
| 133E | Q³ is 3,5-di-MeO-Ph, R³ is I and R¹ is Me. |
| 134E | Q³ is 3,5-di-MeO-Ph, R³ is Me and R¹ is Me. |
| 135E | Q³ is 3,5-di-MeO-Ph, R³ is MeO and R¹ is Me. |
| 136E | Q³ is 3,5-di-MeO-Ph, R³ is MeS and R¹ is Me. |
| 137E | Q³ is 3,5-di-MeO-Ph, R³ is Et and R¹ is Me. |
| 138E | Q³ is 3,5-di-MeO-Ph, R³ is Me and R¹ is Et. |
| 139E | Q³ is 3,5-di-MeO-Ph, R³ is Cl and R¹ is Et. |
| 140E | Q³ is 2-Cl, 3,5-di-MeO-Ph, R³ is Cl and R¹ is Me. |
| 141E | Q³ is 2-Cl, 3,5-di-MeO-Ph, R³ is Cl and R¹ is CFH₂. |
| 142E | Q³ is 2-Cl, 3,5-di-MeO-Ph, R³ is Br and R¹ is Me. |
| 143E | Q³ is 2-Cl, 3,5-di-MeO-Ph, R³ is I and R¹ is Me. |
| 144E | Q³ is 2-Cl, 3,5-di-MeO-Ph, R³ is Me and R¹ is Me. |
| 145E | Q³ is 2-Cl, 3,5-di-MeO-Ph, R³ is MeO and R¹ is Me. |
| 146E | Q³ is 2-Cl, 3,5-di-MeO-Ph, R³ is MeS and R¹ is Me. |
| 147E | Q³ is 2-Cl, 3,5-di-MeO-Ph, R³ is Et and R¹ is Me. |
| 148E | Q³ is 2-Cl, 3,5-di-MeO-Ph, R³ is Me and R¹ is Et. |
| 149E | Q³ is 2-Cl, 3,5-di-MeO-Ph, R³ is Cl and R¹ is Et. |
| 150E | Q³ is 4-Cl, 3,5-di-MeO-Ph, R³ is Cl and R¹ is Me. |
| 151E | Q³ is 4-Cl, 3,5-di-MeO-Ph, R³ is Cl and R¹ is CFH₂. |
| 152E | Q³ is 4-Cl, 3,5-di-MeO-Ph, R³ is Br and R¹ is Me. |
| 153E | Q³ is 4-Cl, 3,5-di-MeO-Ph, R³ is I and R¹ is Me. |
| 154E | Q³ is 4-Cl, 3,5-di-MeO-Ph, R³ is Me and R¹ is Me. |
| 155E | Q³ is 4-Cl, 3,5-di-MeO-Ph, R³ is MeO and R¹ is Me. |
| 156E | Q³ is 4-Cl, 3,5-di-MeO-Ph, R³ is MeS and R¹ is Me. |
| 157E | Q³ is 4-Cl, 3,5-di-MeO-Ph, R³ is Et and R¹ is Me. |
| 158E | Q³ is 4-Cl, 3,5-di-MeO-Ph, R³ is Me and R¹ is Et. |
| 159E | Q³ is 4-Cl, 3,5-di-MeO-Ph, R³ is Cl and R¹ is Et. |
| 160E | Q³ is 4-Cl-Bn, R³ is Cl and R¹ is Me. |
| 161E | Q³ is 4-Cl-Bn, R³ is Cl and R¹ is CFH₂. |
| 162E | Q³ is 4-Cl-Bn, R³ is Br and R¹ is Me. |
| 163E | Q³ is 4-Cl-Bn, R³ is I and R¹ is Me. |
| 164E | Q³ is 4-Cl-Bn, R³ is Me and R¹ is Me. |
| 165E | Q³ is 4-Cl-Bn, R³ is MeO and R¹ is Me. |
| 166E | Q³ is 4-Cl-Bn, R³ is MeS and R¹ is Me. |
| 167E | Q³ is 4-Cl-Bn, R³ is Et and R¹ is Me. |
| 168E | Q³ is 4-Cl-Bn, R³ is Me and R¹ is Et. |
| 169E | Q³ is 4-Cl-Bn, R³ is Cl and R¹ is Et. |
| 170E | Q³ is 4-F-Bn, R³ is Cl and R¹ is Me. |
| 171E | Q³ is 4-F-Bn, R³ is Cl and R¹ is CFH₂. |
| 172E | Q³ is 4-F-Bn, R³ is Br and R¹ is Me. |
| 173E | Q³ is 4-F-Bn, R³ is I and R¹ is Me. |
| 174E | Q³ is 4-F-Bn, R³ is Me and R¹ is Me. |
| 175E | Q³ is 4-F-Bn, R³ is MeO and R¹ is Me. |
| 176E | Q³ is 4-F-Bn, R³ is MeS and R¹ is Me. |
| 177E | Q³ is 4-F-Bn, R³ is Et and R¹ is Me. |
| 178E | Q³ is 4-F-Bn, R³ is Me and R¹ is Et. |
| 179E | Q³ is 4-F-Bn, R³ is Cl and R¹ is Et. |
| 180E | Q³ is 6-Cl-3-pyridinyl, R³ is Cl and R¹ is Me. |
| 181E | Q³ is 6-Cl-3-pyridinyl, R³ is Cl and R¹ is CFH₂. |
| 182E | Q³ is 6-Cl-3-pyridinyl, R³ is Br and R¹ is Me. |
| 183E | Q³ is 6-Cl-3-pyridinyl, R³ is I and R¹ is Me. |
| 184E | Q³ is 6-Cl-3-pyridinyl, R³ is Me and R¹ is Me. |
| 185E | Q³ is 6-Cl-3-pyridinyl, R³ is MeO and R¹ is Me. |
| 186E | Q³ is 6-Cl-3-pyridinyl, R³ is MeS and R¹ is Me. |
| 187E | Q³ is 6-Cl-3-pyridinyl, R³ is Et and R¹ is Me. |
| 188E | Q³ is 6-Cl-3-pyridinyl, R³ is Me and R¹ is Et. |
| 189E | Q³ is 6-Cl-3-pyridinyl, R³ is Cl and R¹ is Et. |
| 190E | Q³ is 6-Me-3-pyridinyl, R³ is Cl and R¹ is Me. |
| 191E | Q³ is 6-Me-3-pyridinyl, R³ is Cl and R¹ is CFH₂. |
| 192E | Q³ is 6-Me-3-pyridinyl, R³ is Br and R¹ is Me. |
| 193E | Q³ is 6-Me-3-pyridinyl, R³ is I and R¹ is Me. |
| 194E | Q³ is 6-Me-3-pyridinyl, R³ is Me and R¹ is Me. |
| 195E | Q³ is 6-Me-3-pyridinyl, R³ is MeO and R¹ is Me. |
| 196E | Q³ is 6-Me-3-pyridinyl, R³ is MeS and R¹ is Me. |
| 197E | Q³ is 6-Me-3-pyridinyl, R³ is Et and R¹ is Me. |
| 198E | Q³ is 6-Me-3-pyridinyl, R³ is Me and R¹ is Et. |
| 199E | Q³ is 6-Me-3-pyridinyl, R³ is Cl and R¹ is Et. |
| 200E | Q³ is 6-MeO-3-pyridinyl, R³ is Cl and R¹ is Me. |
| 201E | Q³ is 6-MeO-3-pyridinyl, R³ is Cl and R¹ is CFH₂. |
| 202E | Q³ is 6-MeO-3-pyridinyl, R³ is Br and R¹ is Me. |
| 203E | Q³ is 6-MeO-3-pyridinyl, R³ is I and R¹ is Me. |
| 204E | Q³ is 6-MeO-3-pyridinyl, R³ is Me and R¹ is Me. |
| 205E | Q³ is 6-MeO-3-pyridinyl, R³ is MeO and R¹ is Me. |
| 206E | Q³ is 6-MeO-3-pyridinyl, R³ is MeS and R¹ is Me. |
| 207E | Q³ is 6-MeO-3-pyridinyl, R³ is Et and R¹ is Me. |
| 208E | Q³ is 6-MeO-3-pyridinyl, R³ is Me and R¹ is Et. |
| 209E | Q³ is 6-MeO-3-pyridinyl, R³ is Cl and R¹ is Et. |
| 210E | Q³ is 6-CF₃-3-pyridinyl, R³ is Cl and R¹ is Me. |
| 211E | Q³ is 6-CF₃-3-pyridinyl, R³ is Cl and R¹ is CFH₂. |
| 212E | Q³ is 6-CF₃-3-pyridinyl, R³ is Br and R¹ is Me. |
| 213E | Q³ is 6-CF₃-3-pyridinyl, R³ is I and R¹ is Me. |
| 214E | Q³ is 6-CF₃-3-pyridinyl, R³ is Me and R¹ is Me. |
| 215E | Q³ is 6-CF₃-3-pyridinyl, R³ is MeO and R¹ is Me. |
| 216E | Q³ is 6-CF₃-3-pyridinyl, R³ is MeS and R¹ is Me. |
| 217E | Q³ is 6-CF₃-3-pyridinyl, R³ is Et and R¹ is Me. |
| 218E | Q³ is 6-CF₃-3-pyridinyl, R³ is Me and R¹ is Et. |
| 219E | Q³ is 6-CF₃-3-pyridinyl, R³ is Cl and R¹ is Et. |
| 220E | Q³ is 6-F-3-pyridinyl, R³ is Cl and R¹ is Me. |
| 221E | Q³ is 6-F-3-pyridinyl, R³ is Cl and R¹ is CFH₂. |
| 222E | Q³ is 6-F-3-pyridinyl, R³ is Br and R¹ is Me. |
| 223E | Q³ is 6-F-3-pyridinyl, R³ is I and R¹ is Me. |
| 224E | Q³ is 6-F-3-pyridinyl, R³ is Me and R¹ is Me. |
| 225E | Q³ is 6-F-3-pyridinyl, R³ is MeO and R¹ is Me. |
| 226E | Q³ is 6-F-3-pyridinyl, R³ is MeS and R¹ is Me. |
| 227E | Q³ is 6-F-3-pyridinyl, R³ is Et and R¹ is Me. |
| 228E | Q³ is 6-F-3-pyridinyl, R³ is Me and R¹ is Et. |
| 229E | Q³ is 6-F-3-pyridinyl, R³ is Cl and R¹ is Et. |
| 230E | Q³ is 5-Cl-3-pyridinyl, R³ is Cl and R¹ is Me. |
| 231E | Q³ is 5-Cl-3-pyridinyl, R³ is Cl and R¹ is CFH₂. |
| 232E | Q³ is 5-Cl-3-pyridinyl, R³ is Br and R¹ is Me. |
| 233E | Q³ is 5-Cl-3-pyridinyl, R³ is I and R¹ is Me. |
| 234E | Q³ is 5-Cl-3-pyridinyl, R³ is Me and R¹ is Me. |
| 235E | Q³ is 5-Cl-3-pyridinyl, R³ is MeO and R¹ is Me. |
| 236E | Q³ is 5-Cl-3-pyridinyl, R³ is MeS and R¹ is Me. |
| 237E | Q³ is 5-Cl-3-pyridinyl, R³ is Et and R¹ is Me. |
| 238E | Q³ is 5-Cl-3-pyridinyl, R³ is Me and R¹ is Et. |
| 239E | Q³ is 5-Cl-3-pyridinyl, R³ is Cl and R¹ is Et. |
| 240E | Q³ is 5-Me-3-pyridinyl, R³ is Cl and R¹ is Me. |
| 241E | Q³ is 5-Me-3-pyridinyl, R³ is Cl and R¹ is CFH₂. |
| 242E | Q³ is 5-Me-3-pyridinyl, R³ is Br and R¹ is Me. |
| 243E | Q³ is 5-Me-3-pyridinyl, R³ is I and R¹ is Me. |
| 244E | Q³ is 5-Me-3-pyridinyl, R³ is Me and R¹ is Me. |
| 245E | Q³ is 5-Me-3-pyridinyl, R³ is MeO and R¹ is Me. |
| 246E | Q³ is 5-Me-3-pyridinyl, R³ is MeS and R¹ is Me. |
| 247E | Q³ is 5-Me-3-pyridinyl, R³ is Et and R¹ is Me. |
| 248E | Q³ is 5-Me-3-pyridinyl, R³ is Me and R¹ is Et. |
| 249E | Q³ is 5-Me-3-pyridinyl, R³ is Cl and R¹ is Et. |
| 250E | Q³ is 5-MeO-3-pyridinyl, R³ is Cl and R¹ is Me. |
| 251E | Q³ is 5-MeO-3-pyridinyl, R³ is Cl and R¹ is CFH₂. |
| 252E | Q³ is 5-MeO-3-pyridinyl, R³ is Br and R¹ is Me. |
| 253E | Q³ is 5-MeO-3-pyridinyl, R³ is I and R¹ is Me. |
| 254E | Q³ is 5-MeO-3-pyridinyl, R³ is Me and R¹ is Me. |
| 255E | Q³ is 5-MeO-3-pyridinyl, R³ is MeO and R¹ is Me. |
| 256E | Q³ is 5-MeO-3-pyridinyl, R³ is MeS and R¹ is Me. |
| 257E | Q³ is 5-MeO-3-pyridinyl, R³ is Et and R¹ is Me. |
| 258E | Q³ is 5-MeO-3-pyridinyl, R³ is Me and R¹ is Et. |
| 259E | Q³ is 5-MeO-3-pyridinyl, R³ is Cl and R¹ is Et. |
| 260E | Q³ is 5-CF₃-3-pyridinyl, R³ is Cl and R¹ is Me. |
| 261E | Q³ is 5-CF₃-3-pyridinyl, R³ is Cl and R¹ is CFH₂. |
| 262E | Q³ is 5-CF₃-3-pyridinyl, R³ is Br and R¹ is Me. |
| 263E | Q³ is 5-CF₃-3-pyridinyl, R³ is I and R¹ is Me. |
| 264E | Q³ is 5-CF₃-3-pyridinyl, R³ is Me and R¹ is Me. |
| 265E | Q³ is 5-CF₃-3-pyridinyl, R³ is MeO and R¹ is Me. |
| 266E | Q³ is 5-CF₃-3-pyridinyl, R³ is MeS and R¹ is Me. |
| 267E | Q³ is 5-CF₃-3-pyridinyl, R³ is Et and R¹ is Me. |
| 268E | Q³ is 5-CF₃-3-pyridinyl, R³ is Me and R¹ is Et. |
| 269E | Q³ is 5-CF₃-3-pyridinyl, R³ is Cl and R¹ is Et. |
| 270E | Q³ is 5-F-3-pyridinyl, R³ is Cl and R¹ is Me. |
| 271E | Q³ is 5-F-3-pyridinyl, R³ is Cl and R¹ is CFH₂. |
| 272E | Q³ is 5-F-3-pyridinyl, R³ is Br and R¹ is Me. |
| 273E | Q³ is 5-F-3-pyridinyl, R³ is I and R¹ is Me. |
| 274E | Q³ is 5-F-3-pyridinyl, R³ is Me and R¹ is Me. |
| 275E | Q³ is 5-F-3-pyridinyl, R³ is MeO and R¹ is Me. |
| 276E | Q³ is 5-F-3-pyridinyl, R³ is MeS and R¹ is Me. |
| 277E | Q³ is 5-F-3-pyridinyl, R³ is Et and R¹ is Me. |
| 278E | Q³ is 5-F-3-pyridinyl, R³ is Me and R¹ is Et. |
| 279E | Q³ is 5-F-3-pyridinyl, R³ is Cl and R¹ is Et. |
| 280E | Q³ is 5-F-3-pyridinyl, R³ is Cl and R¹ is CFH₂. |
| 281E | Q³ is 5-F-3-pyridinyl, R³ is Br and R¹ is Me. |
| 282E | Q³ is 5-F-3-pyridinyl, R³ is I and R¹ is Me. |
| 283E | Q³ is 5-F-3-pyridinyl, R³ is Me and R¹ is Me. |
| 284E | Q³ is 5-F-3-pyridinyl, R³ is MeO and R¹ is Me. |
| 285E | Q³ is 5-F-3-pyridinyl, R³ is MeS and R¹ is Me. |
| 286E | Q³ is 5-F-3-pyridinyl, R³ is Et and R¹ is Me. |
| 287E | Q³ is 5-F-3-pyridinyl, R³ is Me and R¹ is Et. |
| 288E | Q³ is 5-F-3-pyridinyl, R³ is Cl and R¹ is Et. |
| 289E | Q³ is 6-Cl-3-pyridazinyl, R³ is Cl and R¹ is CFH₂. |
| 290E | Q³ is 6-Cl-3-pyridazinyl, R³ is Br and R¹ is Me. |
| 291E | Q³ is 6-Cl-3-pyridazinyl, R³ is I and R¹ is Me. |
| 292E | Q³ is 6-Cl-3-pyridazinyl, R³ is Me and R¹ is Me. |
| 293E | Q³ is 6-Cl-3-pyridazinyl, R³ is MeO and R¹ is Me. |
| 294E | Q³ is 6-Cl-3-pyridazinyl, R³ is MeS and R¹ is Me. |
| 295E | Q³ is 6-Cl-3-pyridazinyl, R³ is Et and R¹ is Me. |
| 296E | Q³ is 6-Cl-3-pyridazinyl, R³ is Me and R¹ is Et. |
| 297E | Q³ is 6-Cl-3-pyridazinyl, R³ is Cl and R¹ is Et. |
| 298E | Q³ is 5-Cl-3-pyridazinyl, R³ is Cl and R¹ is CFH₂. |
| 299E | Q³ is 5-Cl-3-pyridazinyl, R³ is Br and R¹ is Me. |
| 300E | Q³ is 5-Cl-3-pyridazinyl, R³ is I and R¹ is Me. |
| 301E | Q³ is 5-Cl-3-pyridazinyl, R³ is Me and R¹ is Me. |
| 302E | Q³ is 5-Cl-3-pyridazinyl, R³ is MeO and R¹ is Me. |
| 303E | Q³ is 5-Cl-3-pyridazinyl, R³ is MeS and R¹ is Me. |
| 304E | Q³ is 5-Cl-3-pyridazinyl, R³ is Et and R¹ is Me. |
| 305E | Q³ is 5-Cl-3-pyridazinyl, R³ is Me and R¹ is Et. |
| 306E | Q³ is 5-Cl-3-pyridazinyl, R³ is Cl and R¹ is Et. |
| 307E | Q³ is 2-Cl-5-pyrimidinyl, R³ is Cl and R¹ is CFH₂. |
| 308E | Q³ is 2-Cl-5-pyrimidinyl, R³ is Br and R¹ is Me. |
| 309E | Q³ is 2-Cl-5-pyrimidinyl, R³ is I and R¹ is Me. |
| 310E | Q³ is 2-Cl-5-pyrimidinyl, R³ is Me and R¹ is Me. |
| 311E | Q³ is 2-Cl-5-pyrimidinyl, R³ is MeO and R¹ is Me. |
| 312E | Q³ is 2-Cl-5-pyrimidinyl, R³ is MeS and R¹ is Me. |
| 313E | Q³ is 2-Cl-5-pyrimidinyl, R³ is Et and R¹ is Me. |
| 314E | Q³ is 2-Cl-5-pyrimidinyl, R³ is Me and R¹ is Et. |
| 315E | Q³ is 2-Cl-5-pyrimidinyl, R³ is Cl and R¹ is Et. |
| 316E | Q³ is 2-Cl-2-pyrimidinyl, R³ is Cl and R¹ is CFH₂. |
| 317E | Q³ is 2-Cl-2-pyrimidinyl, R³ is Br and R¹ is Me. |
| 318E | Q³ is 2-Cl-2-pyrimidinyl, R³ is I and R¹ is Me. |
| 319E | Q³ is 2-Cl-2-pyrimidinyl, R³ is Me and R¹ is Me. |
| 320E | Q³ is 2-Cl-2-pyrimidinyl, R³ is MeO and R¹ is Me. |
| 321E | Q³ is 2-Cl-2-pyrimidinyl, R³ is MeS and R¹ is Me. |
| 322E | Q³ is 2-Cl-2-pyrimidinyl, R³ is Et and R¹ is Me. |
| 323E | Q³ is 2-Cl-2-pyrimidinyl, R³ is Me and R¹ is Et. |
| 324E | Q³ is 2-Cl-2-pyrimidinyl, R³ is Cl and R¹ is Et. |
| 325E | Q³ is 5-Me-2-thienyl, R³ is Cl and R¹ is CFH₂. |
| 326E | Q³ is 5-Me-2-thienyl, R³ is Br and R¹ is Me. |
| 327E | Q³ is 5-Me-2-thienyl, R³ is I and R¹ is Me. |
| 328E | Q³ is 5-Me-2-thienyl, R³ is Me and R¹ is Me. |
| 329E | Q³ is 5-Me-2-thienyl, R³ is MeO and R¹ is Me. |
| 330E | Q³ is 5-Me-2-thienyl, R³ is MeS and R¹ is Me. |
| 331E | Q³ is 5-Me-2-thienyl, R³ is Et and R¹ is Me. |
| 332E | Q³ is 5-Me-2-thienyl, R³ is Me and R¹ is Et. |
| 333E | Q³ is 5-Me-2-thienyl, R³ is Cl and R¹ is Et. |
| 334E | Q³ is 5-Me-3-thienyl, R³ is Cl and R¹ is CFH₂. |
| 335E | Q³ is 5-Me-3-thienyl, R³ is Br and R¹ is Me. |
| 336E | Q³ is 5-Me-3-thienyl, R³ is I and R¹ is Me. |
| 337E | Q³ is 5-Me-3-thienyl, R³ is Me and R¹ is Me. |
| 338E | Q³ is 5-Me-3-thienyl, R³ is MeO and R¹ is Me. |
| 339E | Q³ is 5-Me-3-thienyl, R³ is MeS and R¹ is Me. |
| 340E | Q³ is 5-Me-3-thienyl, R³ is Et and R¹ is Me. |
| 341E | Q³ is 5-Me-3-thienyl, R³ is Me and R¹ is Et. |
| 342E | Q³ is 5-Me-3-thienyl, R³ is Cl and R¹ is Et. |
| 343E | Q³ is 1-Me-1H-pyrazol-3-yl, R³ is Cl and R¹ is CFH₂. |
| 344E | Q³ is 1-Me-1H-pyrazol-3-yl, R³ is Br and R¹ is Me. |
| 345E | Q³ is 1-Me-1H-pyrazol-3-yl, R³ is I and R¹ is Me. |
| 346E | Q³ is 1-Me-1H-pyrazol-3-yl, R³ is Me and R¹ is Me. |
| 347E | Q³ is 1-Me-1H-pyrazol-3-yl, R³ is MeO and R¹ is Me. |
| 348E | Q³ is 1-Me-1H-pyrazol-3-yl, R³ is MeS and R¹ is Me. |
| 349E | Q³ is 1-Me-1H-pyrazol-3-yl, R³ is Et and R¹ is Me. |
| 350E | Q³ is 1-Me-1H-pyrazol-3-y1,, R³ is Me and R¹ is Et. |
| 351E | Q³ is 1-Me-1H-pyrazol-3-yl, R³ is Cl and R¹ is Et. |
| 352E | Q³ is 1-Me-1H-pyrazol-4-yl, R³ is Cl and R¹ is CFH₂. |
| 353E | Q³ is 1-Me-1H-pyrazol-4-yl, R³ is Br and R¹ is Me. |
| 354E | Q³ is 1-Me-1H-pyrazol-4-yl, R³ is I and R¹ is Me. |
| 355E | Q³ is 1-Me-1H-pyrazol-4-yl, R³ is Me and R¹ is Me. |
| 356E | Q³ is 1-Me-1H-pyrazol-4-yl, R³ is MeO and R¹ is Me. |
| 357E | Q³ is 1-Me-1H-pyrazol-4-yl, R³ is MeS and R¹ is Me. |
| 358E | Q³ is 1-Me-1H-pyrazol-4-yl, R³ is Et and R¹ is Me. |
| 359E | Q³ is 1-Me-1H-pyrazol-4-yl, R³ is Me and R¹ is Et. |
| 360E | Q³ is 1-Me-1H-pyrazol-4-yl, R³ is Cl and R¹ is Et. |
| 361E | Q³ is 2-Me-5-thiazolyl, R³ is Cl and R¹ is CFH₂. |
| 362E | Q³ is 2-Me-5-thiazolyl, R³ is Br and R¹ is Me. |
| 363E | Q³ is 2-Me-5-thiazolyl, R³ is I and R¹ is Me. |
| 364E | Q³ is 2-Me-5-thiazolyl, R³ is Me and R¹ is Me. |
| 365E | Q³ is 2-Me-5-thiazolyl, R³ is MeO and R¹ is Me. |
| 366E | Q³ is 2-Me-5-thiazolyl, R³ is MeS and R¹ is Me. |
| 367E | Q³ is 2-Me-5-thiazolyl, R³ is Et and R¹ is Me. |
| 368E | Q³ is 2-Me-5-thiazolyl, R³ is Me and R¹ is Et. |
| 369E | Q³ is 2-Me-5-thiazolyl, R³ is Cl and R¹ is Et. |
| 370E | Q³ is 2-Cl-5-thiazolyl, R³ is Cl and R¹ is CFH₂. |
| 371E | Q³ is 2-Cl-5-thiazolyl, R³ is Br and R¹ is Me. |
| 372E | Q³ is 2-Cl-5-thiazolyl, R³ is I and R¹ is Me. |
| 373E | Q³ is 2-Cl-5-thiazolyl, R³ is Me and R¹ is Me. |
| 374E | Q³ is 2-Cl-5-thiazolyl, R³ is MeO and R¹ is Me. |
| 375E | Q³ is 2-Cl-5-thiazolyl, R³ is MeS and R¹ is Me. |
| 376E | Q³ is 2-Cl-5-thiazolyl, R³ is Et and R¹ is Me. |
| 377E | Q³ is 2-Cl-5-thiazolyl, R³ is Me and R¹ is Et. |
| 378E | Q³ is 2-Cl-5-thiazolyl, R³ is Cl and R¹ is Et. |
| 379E | Q³ is 5-Me-3-isothiazolyl, R³ is Cl and R¹ is CFH₂. |
| 380E | Q³ is 5-Me-3-isothiazolyl, R³ is Br and R¹ is Me. |
| 381E | Q³ is 5-Me-3-isothiazolyl, R³ is I and R¹ is Me. |
| 382E | Q³ is 5-Me-3-isothiazolyl, R³ is Me and R¹ is Me. |
| 383E | Q³ is 5-Me-3-isothiazolyl, R³ is MeO and R¹ is Me. |
| 384E | Q³ is 5-Me-3-isothiazolyl, R³ is MeS and R¹ is Me. |
| 385E | Q³ is 5-Me-3-isothiazolyl, R³ is Et and R¹ is Me. |
| 386E | Q³ is 5-Me-3-isothiazolyl, R³ is Me and R¹ is Et. |
| 387E | Q³ is 5-Me-3-isothiazolyl, R³ is Cl and R¹ is Et. |

TABLE 6

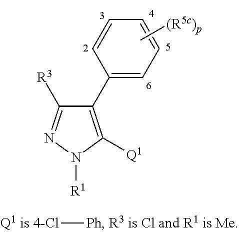

| (R^{5c})ₚ | (R^{5c})ₚ | (R^{5c})ₚ | (R^{5c})ₚ | (R^{5c})ₚ |
|---|---|---|---|---|
| 2,6-di-F | 2,4-di-Cl | 4-CN, 2,6-di-F | 2-CF₃, 4-F | 2-Cl, 4-NO₂ |
| 2,4,6-tri-F | 2-Cl, 4,6-di-F | 2,6-di-F, 4-Me | 2-CF₂HO, 4-F | 2-NO₂, 4-F |
| 2,3,6-tri-F | 2-Cl, 6-F | 2-Cl, 5-CF₃ | 2-CN, 6-F | 2,3,4,5,6-penta-F |
| 2,4,5-tri-F | 2-Br, 6-F | 2-Cl, 4-Me | 2,5-di-Cl | 2,5-di-Cl, 4-F |
| 2,3,4-tri-F | 2-F, 6-CF₃ | 2-Cl, 4-MeO | 2-CF₃, 4-MeO | 2,3-di-Cl, 4-F |
| 2-Cl, 4-F | 2-F, 6-CF₂HO | 2-Br, 4-MeO | 2-F, 6-Me | 2-Cl, 5-CN |
| 2-Br-4-F | 2-I, 4-F | 2,6-di-F, 3-Cl | 2,6-di-F, 3-Me | 2,4-di-F, 5-CN |
| 2,4-di-F | 4-Cl, 2,6-di-F | 2,6-di-F, 3-CN | 2-CF₃ | 2-Cl, 6-F, 3-MeO |
| 2,6-di-Cl | 2,6-di-F, 4-MeO | 2,6-di-F, 3-MeO | 2-CF₃O | |
| 2,6-di-F, 4-EtO | 2-Cl, 6-F, 4-MeO | 2,6-di-F, 4-CF₂HO | 2,6-di-F, 4-NO₂ | |
| 2-Cl, 3,6-di-F | 2-Cl, 6-F, 5-MeO | 2,6-di-F, 3-CF₂HO | 2,6-di-F, 3-EtO | |

The present disclosure also includes Tables 1F through 934F, each of which is constructed the same as Table 6 above except that the row heading in Table 6 (i.e. “Q¹ is 4-Cl-Ph, R³ is Cl and R¹ is Me”) is replaced with the respective row heading shown below. Thus, for example, in Table 1F the row heading is “Q³ is 4-Cl-Ph, R³ is Cl and R¹ is CFH₂” and (R^{5c})ₚ is as defined in Table 6 above. Tables 2F through 934F are constructed similarly.

| Table Number | Row Heading |
|---|---|
| 1F | Q³ is 4-Cl-Ph, R³ is Cl and R¹ is CFH₂ |
| 2F | Q³ is 4-Cl-Ph, R³ is Br and R¹ is Me. |
| 3F | Q³ is 4-Cl-Ph, R³ is I and R¹ is Me. |
| 4F | Q³ is 4-Cl-Ph, R³ is Me and R¹ is Me. |
| 5F | Q³ is 4-Cl-Ph, R³ is MeO and R¹ is Me. |
| 6F | Q³ is 4-Cl-Ph, R³ is MeS and R¹ is Me. |
| 7F | Q³ is 4-Cl-Ph, R³ is Et and R¹ is Me. |
| 8F | Q³ is 4-Cl-Ph, R³ is Me and R¹ is Et. |
| 9F | Q³ is 4-Cl-Ph, R³ is Cl and R¹ is Et. |
| 10F | Q³ is 3-Cl-Ph, R³ is Cl and R¹ is Me. |
| 11F | Q³ is 3-Cl-Ph, R³ is Cl and R¹ is CFH₂. |
| 12F | Q³ is 3-Cl-Ph, R³ is Br and R¹ is Me. |
| 13F | Q³ is 3-Cl-Ph, R³ is I and R¹ is Me. |
| 14F | Q³ is 3-Cl-Ph, R³ is Me and R¹ is Me. |
| 15F | Q³ is 3-Cl-Ph, R³ is MeO and R¹ is Me. |
| 16F | Q³ is 3-Cl-Ph, R³ is MeS and R¹ is Me. |
| 17F | Q³ is 3-Cl-Ph, R³ is Et and R¹ is Me. |
| 18F | Q³ is 3-Cl-Ph, R³ is Me and R¹ is Et. |
| 19F | Q³ is 3-Cl-Ph, R³ is Cl and R¹ is Et. |
| 20F | Q³ is 4-F-Ph, R³ is Cl and R¹ is Me. |
| 21F | Q³ is 4-F-Ph, R³ is Cl and R¹ is CFH₂. |
| 22F | Q³ is 4-F-Ph, R³ is Br and R¹ is Me. |
| 23F | Q³ is 4-F-Ph, R³ is I and R¹ is Me. |
| 24F | Q³ is 4-F-Ph, R³ is Me and R¹ is Me. |
| 25F | Q³ is 4-F-Ph, R³ is MeO and R¹ is Me. |
| 26F | Q³ is 4-F-Ph, R³ is MeS and R¹ is Me. |
| 27F | Q³ is 4-F-Ph, R³ is Et and R¹ is Me. |
| 28F | Q³ is 4-F-Ph, R³ is Me and R¹ is Et. |
| 29F | Q³ is 4-F-Ph, R³ is Cl and R¹ is Et. |
| 30F | Q³ is 3-F-Ph, R³ is Cl and R¹ is Me. |
| 31F | Q³ is 3-F-Ph, R³ is Cl and R¹ is CFH₂. |
| 32F | Q³ is 3-F-Ph, R³ is Br and R¹ is Me. |
| 33F | Q³ is 3-F-Ph, R³ is I and R¹ is Me. |
| 34F | Q³ is 3-F-Ph, R³ is Me and R¹ is Me. |
| 35F | Q³ is 3-F-Ph, R³ is MeO and R¹ is Me. |
| 36F | Q³ is 3-F-Ph, R³ is MeS and R¹ is Me. |
| 37F | Q³ is 3-F-Ph, R³ is Et and R¹ is Me. |
| 38F | Q³ is 3-F-Ph, R³ is Me and R¹ is Et. |
| 39F | Q³ is 3-F-Ph, R³ is Cl and R¹ is Et. |
| 40F | Q³ is 3-CF₂HO-Ph, R³ is Cl and R¹ is Me. |
| 41F | Q³ is 3-CF₂HO-Ph, R³ is Cl and R¹ is CFH₂. |
| 42F | Q³ is 3-CF₂HO-Ph, R³ is Br and R¹ is Me. |
| 43F | Q³ is 3-CF₂HO-Ph, R³ is I and R¹ is Me. |
| 44F | Q³ is 3-CF₂HO-Ph, R³ is Me and R¹ is Me. |
| 45F | Q³ is 3-CF₂HO-Ph, R³ is MeO and R¹ is Me. |
| 46F | Q³ is 3-CF₂HO-Ph, R³ is MeS and R¹ is Me. |
| 47F | Q³ is 3-CF₂HO-Ph, R³ is Et and R¹ is Me. |
| 48F | Q³ is 3-CF₂HO-Ph, R³ is Me and R¹ is Et. |
| 49F | Q³ is 3-CF₂HO-Ph, R³ is Cl and R¹ is Et. |
| 50F | Q³ is 4-Me-Ph, R³ is Cl and R¹ is Me. |
| 51F | Q³ is 4-Me-Ph, R³ is Cl and R¹ is CFH₂. |
| 52F | Q³ is 4-Me-Ph, R³ is Br and R¹ is Me. |
| 53F | Q³ is 4-Me-Ph, R³ is I and R¹ is Me. |
| 54F | Q³ is 4-Me-Ph, R³ is Me and R¹ is Me. |
| 55F | Q³ is 4-Me-Ph, R³ is MeO and R¹ is Me. |
| 56F | Q³ is 4-Me-Ph, R³ is MeS and R¹ is Me. |
| 57F | Q³ is 4-Me-Ph, R³ is Et and R¹ is Me. |
| 58F | Q³ is 4-Me-Ph, R³ is Me and R¹ is Et. |
| 59F | Q³ is 4-Me-Ph, R³ is Cl and R¹ is Et. |
| 60F | Q³ is 3-Me-Ph, R³ is Cl and R¹ is Me. |
| 61F | Q³ is 3-Me-Ph, R³ is Cl and R¹ is CFH₂. |
| 62F | Q³ is 3-Me-Ph, R³ is Br and R¹ is Me. |
| 63F | Q³ is 3-Me-Ph, R³ is I and R¹ is Me. |
| 64F | Q³ is 3-Me-Ph, R³ is Me and R¹ is Me. |
| 65F | Q³ is 3-Me-Ph, R³ is MeO and R¹ is Me. |
| 66F | Q³ is 3-Me-Ph, R³ is MeS and R¹ is Me. |
| 67F | Q³ is 3-Me-Ph, R³ is Et and R¹ is Me. |
| 68F | Q³ is 3-Me-Ph, R³ is Me and R¹ is Et. |
| 69F | Q³ is 3-Me-Ph, R³ is Cl and R¹ is Et. |
| 70F | Q³ is 4-Et-Ph, R³ is Cl and R¹ is Me. |
| 71F | Q³ is 4-Et-Ph, R³ is Cl and R¹ is CFH₂. |
| 72F | Q³ is 4-Et-Ph, R³ is Br and R¹ is Me. |
| 73F | Q³ is 4-Et-Ph, R³ is I and R¹ is Me. |
| 74F | Q³ is 4-Et-Ph, R³ is Me and R¹ is Me. |
| 75F | Q³ is 4-Et-Ph, R³ is MeO and R¹ is Me. |
| 76F | Q³ is 4-Et-Ph, R³ is MeS and R¹ is Me. |
| 77F | Q³ is 4-Et-Ph, R³ is Et and R¹ is Me. |
| 78F | Q³ is 4-Et-Ph, R³ is Me and R¹ is Et. |
| 79F | Q³ is 4-Et-Ph, R³ is Cl and R¹ is Et. |
| 80F | Q³ is 4-Cl-3-F-Ph, R³ is Cl and R¹ is Me. |
| 81F | Q³ is 4-Cl-3-F-Ph, R³ is Cl and R¹ is CFH₂. |
| 82F | Q³ is 4-Cl-3-F-Ph, R³ is Br and R¹ is Me. |
| 83F | Q³ is 4-Cl-3-F-Ph, R³ is I and R¹ is Me. |
| 84F | Q³ is 4-Cl-3-F-Ph, R³ is Me and R¹ is Me. |
| 85F | Q³ is 4-Cl-3-F-Ph, R³ is MeO and R¹ is Me. |
| 86F | Q³ is 4-Cl-3-F-Ph, R³ is MeS and R¹ is Me. |
| 87F | Q³ is 4-Cl-3-F-Ph, R³ is Et and R¹ is Me. |
| 88F | Q³ is 4-Cl-3-F-Ph, R³ is Me and R¹ is Et. |
| 89F | Q³ is 4-Cl-3-F-Ph, R³ is Cl and R¹ is Et. |
| 90F | Q³ is 2-Cl, 4-F-Ph, R³ is Cl and R¹ is Me. |
| 91F | Q³ is 2-Cl, 4-F-Ph, R³ is Cl and R¹ is CFH₂. |
| 92F | Q³ is 2-Cl, 4-F-Ph, R³ is Br and R¹ is Me. |
| 93F | Q³ is 2-Cl, 4-F-Ph, R³ is I and R¹ is Me. |
| 94F | Q³ is 2-Cl, 4-F-Ph, R³ is Me and R¹ is Me. |
| 95F | Q³ is 2-Cl, 4-F-Ph, R³ is MeO and R¹ is Me. |
| 96F | Q³ is 2-Cl, 4-F-Ph, R³ is MeS and R¹ is Me. |
| 97F | Q³ is 2-Cl, 4-F-Ph, R³ is Et and R¹ is Me. |
| 98F | Q³ is 2-Cl, 4-F-Ph, R³ is Me and R¹ is Et. |
| 99F | Q³ is 2-Cl, 4-F-Ph, R³ is Cl and R¹ is Et. |
| 100F | Q³ is 4-F, 3-Me-Ph, R³ is Cl and R¹ is Me. |
| 101F | Q³ is 4-F, 3-Me-Ph, R³ is Cl and R¹ is CFH₂. |
| 102F | Q³ is 4-F, 3-Me-Ph, R³ is Br and R¹ is Me. |
| 103F | Q³ is 4-F, 3-Me-Ph, R³ is I and R¹ is Me. |
| 104F | Q³ is 4-F, 3-Me-Ph, R³ is Me and R¹ is Me. |
| 105F | Q³ is 4-F, 3-Me-Ph, R³ is MeO and R¹ is Me. |
| 106F | Q³ is 4-F, 3-Me-Ph, R³ is MeS and R¹ is Me. |
| 107F | Q³ is 4-F, 3-Me-Ph, R³ is Et and R¹ is Me. |
| 108F | Q³ is 4-F, 3-Me-Ph, R³ is Me and R¹ is Et. |
| 109F | Q³ is 4-F, 3-Me-Ph, R³ is Cl and R¹ is Et. |
| 110F | Q³ is 3,4-di-F-Ph, R³ is Cl and R¹ is Me. |
| 111F | Q³ is 3,4-di-F-Ph, R³ is Cl and R¹ is CFH₂. |
| 112F | Q³ is 3,4-di-F-Ph, R³ is Br and R¹ is Me. |
| 113F | Q³ is 3,4-di-F-Ph, R³ is I and R¹ is Me. |
| 114F | Q³ is 3,4-di-F-Ph, R³ is Me and R¹ is Me. |
| 115F | Q³ is 3,4-di-F-Ph, R³ is MeO and R¹ is Me. |
| 116F | Q³ is 3,4-di-F-Ph, R³ is MeS and R¹ is Me. |
| 117F | Q³ is 3,4-di-F-Ph, R³ is Et and R¹ is Me. |
| 118F | Q³ is 3,4-di-F-Ph, R³ is Me and R¹ is Et. |
| 119F | Q³ is 3,4-di-F-Ph, R³ is Cl and R¹ is Et. |
| 120F | Q³ is 3,4-di-Cl-Ph, R³ is Cl and R¹ is Me. |
| 121F | Q³ is 3,4-di-Cl-Ph, R³ is Cl and R¹ is CFH₂. |
| 122F | Q³ is 3,4-di-Cl-Ph, R³ is Br and R¹ is Me. |
| 123F | Q³ is 3,4-di-Cl-Ph, R³ is I and R¹ is Me. |
| 124F | Q³ is 3,4-di-Cl-Ph, R³ is Me and R¹ is Me. |
| 125F | Q³ is 3,4-di-Cl-Ph, R³ is MeO and R¹ is Me. |
| 126F | Q³ is 3,4-di-Cl-Ph, R³ is MeS and R¹ is Me. |
| 127F | Q³ is 3,4-di-Cl-Ph, R³ is Et and R¹ is Me. |
| 128F | Q³ is 3,4-di-Cl-Ph, R³ is Me and R¹ is Et. |
| 129F | Q³ is 3,4-di-Cl-Ph, R³ is Cl and R¹ is Et. |
| 130F | Q³ is 3,5-di-MeO-Ph, R³ is Cl and R¹ is Me. |
| 131F | Q³ is 3,5-di-MeO-Ph, R³ is Cl and R¹ is CFH₂. |
| 132F | Q³ is 3,5-di-MeO-Ph, R³ is Br and R¹ is Me. |
| 133F | Q³ is 3,5-di-MeO-Ph, R³ is I and R¹ is Me. |
| 134F | Q³ is 3,5-di-MeO-Ph, R³ is Me and R¹ is Me. |
| 135F | Q³ is 3,5-di-MeO-Ph, R³ is MeO and R¹ is Me. |
| 136F | Q³ is 3,5-di-MeO-Ph, R³ is MeS and R¹ is Me. |
| 137F | Q³ is 3,5-di-MeO-Ph, R³ is Et and R¹ is Me. |
| 138F | Q³ is 3,5-di-MeO-Ph, R³ is Me and R¹ is Et. |
| 139F | Q³ is 3,5-di-MeO-Ph, R³ is Cl and R¹ is Et. |
| 140F | Q³ is 2-Cl, 3,5-di-MeO-Ph, R³ is Cl and R¹ is Me. |
| 141F | Q³ is 2-Cl, 3,5-di-MeO-Ph, R³ is Cl and R¹ is CFH₂. |
| 142F | Q³ is 2-Cl, 3,5-di-MeO-Ph, R³ is Br and R¹ is Me. |
| 143F | Q³ is 2-Cl, 3,5-di-MeO-Ph, R³ is I and R¹ is Me. |
| 144F | Q³ is 2-Cl, 3,5-di-MeO-Ph, R³ is Me and R¹ is Me. |
| 145F | Q³ is 2-Cl, 3,5-di-MeO-Ph, R³ is MeO and R¹ is Me. |
| 146F | Q³ is 2-Cl, 3,5-di-MeO-Ph, R³ is MeS and R¹ is Me. |
| 147F | Q³ is 2-Cl, 3,5-di-MeO-Ph, R³ is Et and R¹ is Me. |
| 148F | Q³ is 2-Cl, 3,5-di-MeO-Ph, R³ is Me and R¹ is Et. |
| 149F | Q³ is 2-Cl, 3,5-di-MeO-Ph, R³ is Cl and R¹ is Et. |
| 150F | Q³ is 4-Cl, 3,5-di-MeO-Ph, R³ is Cl and R¹ is Me. |
| 151F | Q³ is 4-Cl, 3,5-di-MeO-Ph, R³ is Cl and R¹ is CFH₂. |
| 152F | Q³ is 4-Cl, 3,5-di-MeO-Ph, R³ is Br and R¹ is Me. |
| 153F | Q³ is 4-Cl, 3,5-di-MeO-Ph, R³ is I and R¹ is Me. |
| 154F | Q³ is 4-Cl, 3,5-di-MeO-Ph, R³ is Me and R¹ is Me. |
| 155F | Q³ is 4-Cl, 3,5-di-MeO-Ph, R³ is MeO and R¹ is Me. |
| 156F | Q³ is 4-Cl, 3,5-di-MeO-Ph, R³ is MeS and R¹ is Me. |
| 157F | Q³ is 4-Cl, 3,5-di-MeO-Ph, R³ is Et and R¹ is Me. |
| 158F | Q³ is 4-Cl, 3,5-di-MeO-Ph, R³ is Me and R¹ is Et. |
| 159F | Q³ is 4-Cl, 3,5-di-MeO-Ph, R³ is Cl and R¹ is Et. |
| 160F | Q³ is 4-Cl-Bn, R³ is Cl and R¹ is Me. |
| 161F | Q³ is 4-Cl-Bn, R³ is Cl and R¹ is CFH₂. |
| 162F | Q³ is 4-Cl-Bn, R³ is Br and R¹ is Me. |
| 163F | Q³ is 4-Cl-Bn, R³ is I and R¹ is Me. |
| 164F | Q³ is 4-Cl-Bn, R³ is Me and R¹ is Me. |
| 165F | Q³ is 4-Cl-Bn, R³ is MeO and R¹ is Me. |
| 166F | Q³ is 4-Cl-Bn, R³ is MeS and R¹ is Me. |
| 167F | Q³ is 4-Cl-Bn, R³ is Et and R¹ is Me. |
| 168F | Q³ is 4-Cl-Bn, R³ is Me and R¹ is Et. |
| 169F | Q³ is 4-Cl-Bn, R³ is Cl and R¹ is Et. |
| 170F | Q³ is 4-F-Bn, R³ is Cl and R¹ is Me. |
| 171F | Q³ is 4-F-Bn, R³ is Cl and R¹ is CFH₂. |
| 172F | Q³ is 4-F-Bn, R³ is Br and R¹ is Me. |
| 173F | Q³ is 4-F-Bn, R³ is I and R¹ is Me. |
| 174F | Q³ is 4-F-Bn, R³ is Me and R¹ is Me. |
| 175F | Q³ is 4-F-Bn, R³ is MeO and R¹ is Me. |
| 176F | Q³ is 4-F-Bn, R³ is MeS and R¹ is Me. |
| 177F | Q³ is 4-F-Bn, R³ is Et and R¹ is Me. |
| 178F | Q³ is 4-F-Bn, R³ is Me and R¹ is Et. |
| 179F | Q³ is 4-F-Bn, R³ is Cl and R¹ is Et. |
| 180F | Q³ is 6-Cl-3-pyridinyl, R³ is Cl and R¹ is Me. |
| 181F | Q³ is 6-Cl-3-pyridinyl, R³ is Cl and R¹ is CFH₂. |
| 182F | Q³ is 6-Cl-3-pyridinyl, R³ is Br and R¹ is Me. |
| 183F | Q³ is 6-Cl-3-pyridinyl, R³ is I and R¹ is Me. |
| 184F | Q³ is 6-Cl-3-pyridinyl, R³ is Me and R¹ is Me. |
| 185F | Q³ is 6-Cl-3-pyridinyl, R³ is MeO and R¹ is Me. |
| 186F | Q³ is 6-Cl-3-pyridinyl, R³ is MeS and R¹ is Me. |
| 187F | Q³ is 6-Cl-3-pyridinyl, R³ is Et and R¹ is Me. |
| 188F | Q³ is 6-Cl-3-pyridinyl, R³ is Me and R¹ is Et. |
| 189F | Q³ is 6-Cl-3-pyridinyl, R³ is Cl and R¹ is Et. |
| 190F | Q³ is 6-Me-3-pyridinyl, R³ is Cl and R¹ is Me. |
| 191F | Q³ is 6-Me-3-pyridinyl, R³ is Cl and R¹ is CFH₂. |
| 192F | Q³ is 6-Me-3-pyridinyl, R³ is Br and R¹ is Me. |
| 193F | Q³ is 6-Me-3-pyridinyl, R³ is I and R¹ is Me. |
| 194F | Q³ is 6-Me-3-pyridinyl, R³ is Me and R¹ is Me. |
| 195F | Q³ is 6-Me-3-pyridinyl, R³ is MeO and R¹ is Me. |
| 196F | Q³ is 6-Me-3-pyridinyl, R³ is MeS and R¹ is Me. |
| 197F | Q³ is 6-Me-3-pyridinyl, R³ is Et and R¹ is Me. |
| 198F | Q³ is 6-Me-3-pyridinyl, R³ is Me and R¹ is Et. |
| 199F | Q³ is 6-Me-3-pyridinyl, R³ is Cl and R¹ is Et. |
| 200F | Q³ is 6-MeO-3-pyridinyl, R³ is Cl and R¹ is Me. |
| 201F | Q³ is 6-MeO-3-pyridinyl, R³ is Cl and R¹ is CFH₂. |
| 202F | Q³ is 6-MeO-3-pyridinyl, R³ is Br and R¹ is Me. |
| 203F | Q³ is 6-MeO-3-pyridinyl, R³ is I and R¹ is Me. |
| 204F | Q³ is 6-MeO-3-pyridinyl, R³ is Me and R¹ is Me. |
| 205F | Q³ is 6-MeO-3-pyridinyl, R³ is MeO and R¹ is Me. |
| 206F | Q³ is 6-MeO-3-pyridinyl, R³ is MeS and R¹ is Me. |
| 207F | Q³ is 6-MeO-3-pyridinyl, R³ is Et and R¹ is Me. |
| 208F | Q³ is 6-MeO-3-pyridinyl, R³ is Me and R¹ is Et. |
| 209F | Q³ is 6-MeO-3-pyridinyl, R³ is Cl and R¹ is Et. |
| 210F | Q³ is 6-CF₃-3-pyridinyl, R³ is Cl and R¹ is Me. |
| 211F | Q³ is 6-CF₃-3-pyridinyl, R³ is Cl and R¹ is CFH₂. |
| 212F | Q³ is 6-CF₃-3-pyridinyl, R³ is Br and R¹ is Me. |
| 213F | Q³ is 6-CF₃-3-pyridinyl, R³ is I and R¹ is Me. |
| 214F | Q³ is 6-CF₃-3-pyridinyl, R³ is Me and R¹ is Me. |
| 215F | Q³ is 6-CF₃-3-pyridinyl, R³ is MeO and R¹ is Me. |
| 216F | Q³ is 6-CF₃-3-pyridinyl, R³ is MeS and R¹ is Me. |
| 217F | Q³ is 6-CF₃-3-pyridinyl, R³ is Et and R¹ is Me. |
| 218F | Q³ is 6-CF₃-3-pyridinyl, R³ is Me and R¹ is Et. |
| 219F | Q³ is 6-CF₃-3-pyridinyl, R³ is Cl and R¹ is Et. |
| 220F | Q³ is 6-F-3-pyridinyl, R³ is Cl and R¹ is Me. |
| 221F | Q³ is 6-F-3-pyridinyl, R³ is Cl and R¹ is CFH₂. |
| 222F | Q³ is 6-F-3-pyridinyl, R³ is Br and R¹ is Me. |
| 223F | Q³ is 6-F-3-pyridinyl, R³ is I and R¹ is Me. |
| 224F | Q³ is 6-F-3-pyridinyl, R³ is Me and R¹ is Me. |
| 225F | Q³ is 6-F-3-pyridinyl, R³ is MeO and R¹ is Me. |
| 226F | Q³ is 6-F-3-pyridinyl, R³ is MeS and R¹ is Me. |
| 227F | Q³ is 6-F-3-pyridinyl, R³ is Et and R¹ is Me. |
| 228F | Q³ is 6-F-3-pyridinyl, R³ is Me and R¹ is Et. |
| 229F | Q³ is 6-F-3-pyridinyl, R³ is Cl and R¹ is Et. |
| 230F | Q³ is 5-Cl-3-pyridinyl, R³ is Cl and R¹ is Me. |
| 231F | Q³ is 5-Cl-3-pyridinyl, R³ is Cl and R¹ is CFH₂. |
| 232F | Q³ is 5-Cl-3-pyridinyl, R³ is Br and R¹ is Me. |
| 233F | Q³ is 5-Cl-3-pyridinyl, R³ is I and R¹ is Me. |
| 234F | Q³ is 5-Cl-3-pyridinyl, R³ is Me and R¹ is Me. |
| 235F | Q³ is 5-Cl-3-pyridinyl, R³ is MeO and R¹ is Me. |
| 236F | Q³ is 5-Cl-3-pyridinyl, R³ is MeS and R¹ is Me. |
| 237F | Q³ is 5-Cl-3-pyridinyl, R³ is Et and R¹ is Me. |
| 238F | Q³ is 5-Cl-3-pyridinyl, R³ is Me and R¹ is Et. |
| 239F | Q³ is 5-Cl-3-pyridinyl, R³ is Cl and R¹ is Et. |
| 240F | Q³ is 5-Me-3-pyridinyl, R³ is Cl and R¹ is Me. |
| 241F | Q³ is 5-Me-3-pyridinyl, R³ is Cl and R¹ is CFH₂. |
| 242F | Q³ is 5-Me-3-pyridinyl, R³ is Br and R¹ is Me. |
| 243F | Q³ is 5-Me-3-pyridinyl, R³ is I and R¹ is Me. |
| 244F | Q³ is 5-Me-3-pyridinyl, R³ is Me and R¹ is Me. |
| 245F | Q³ is 5-Me-3-pyridinyl, R³ is MeO and R¹ is Me. |
| 246F | Q³ is 5-Me-3-pyridinyl, R³ is MeS and R¹ is Me. |
| 247F | Q³ is 5-Me-3-pyridinyl, R³ is Et and R¹ is Me. |
| 248F | Q³ is 5-Me-3-pyridinyl, R³ is Me and R¹ is Et. |
| 249F | Q³ is 5-Me-3-pyridinyl, R³ is Cl and R¹ is Et. |
| 250F | Q³ is 5-MeO-3-pyridinyl, R³ is Cl and R¹ is Me. |
| 251F | Q³ is 5-MeO-3-pyridinyl, R³ is Cl and R¹ is CFH₂. |
| 252F | Q³ is 5-MeO-3-pyridinyl, R³ is Br and R¹ is Me. |
| 253F | Q³ is 5-MeO-3-pyridinyl, R³ is I and R¹ is Me. |
| 254F | Q³ is 5-MeO-3-pyridinyl, R³ is Me and R¹ is Me. |
| 255F | Q³ is 5-MeO-3-pyridinyl, R³ is MeO and R¹ is Me. |
| 256F | Q³ is 5-MeO-3-pyridinyl, R³ is MeS and R¹ is Me. |
| 257F | Q³ is 5-MeO-3-pyridinyl, R³ is Et and R¹ is Me. |
| 258F | Q³ is 5-MeO-3-pyridinyl, R³ is Me and R¹ is Et. |
| 259F | Q³ is 5-MeO-3-pyridinyl, R³ is Cl and R¹ is Et. |
| 260F | Q³ is 5-CF₃-3-pyridinyl, R³ is Cl and R¹ is Me. |
| 261F | Q³ is 5-CF₃-3-pyridinyl, R³ is Cl and R¹ is CFH₂. |
| 262F | Q³ is 5-CF₃-3-pyridinyl, R³ is Br and R¹ is Me. |
| 263F | Q³ is 5-CF₃-3-pyridinyl, R³ is I and R¹ is Me. |
| 264F | Q³ is 5-CF₃-3-pyridinyl, R³ is Me and R¹ is Me. |
| 265F | Q³ is 5-CF₃-3-pyridinyl, R³ is MeO and R¹ is Me. |
| 266F | Q³ is 5-CF₃-3-pyridinyl, R³ is MeS and R¹ is Me. |
| 267F | Q³ is 5-CF₃-3-pyridinyl, R³ is Et and R¹ is Me. |
| 268F | Q³ is 5-CF₃-3-pyridinyl, R³ is Me and R¹ is Et. |
| 269F | Q³ is 5-CF₃-3-pyridinyl, R³ is Cl and R¹ is Et. |
| 270F | Q³ is 5-F-3-pyridinyl, R³ is Cl and R¹ is Me. |
| 271F | Q³ is 5-F-3-pyridinyl, R³ is Cl and R¹ is CFH₂. |
| 272F | Q³ is 5-F-3-pyridinyl, R³ is Br and R¹ is Me. |
| 273F | Q³ is 5-F-3-pyridinyl, R³ is I and R¹ is Me. |
| 274F | Q³ is 5-F-3-pyridinyl, R³ is Me and R¹ is Me. |
| 275F | Q³ is 5-F-3-pyridinyl, R³ is MeO and R¹ is Me. |
| 276F | Q³ is 5-F-3-pyridinyl, R³ is MeS and R¹ is Me. |
| 277F | Q³ is 5-F-3-pyridinyl, R³ is Et and R¹ is Me. |
| 278F | Q³ is 5-F-3-pyridinyl, R³ is Me and R¹ is Et. |
| 279F | Q³ is 5-F-3-pyridinyl, R³ is Cl and R¹ is Et. |
| 280F | Q³ is 5-F-3-pyridinyl, R³ is Cl and R¹ is CFH₂. |
| 281F | Q³ is 5-F-3-pyridinyl, R³ is Br and R¹ is Me. |
| 282F | Q³ is 5-F-3-pyridinyl, R³ is I and R¹ is Me. |
| 283F | Q³ is 5-F-3-pyridinyl, R³ is Me and R¹ is Me. |
| 284F | Q³ is 5-F-3-pyridinyl, R³ is MeO and R¹ is Me. |
| 285F | Q³ is 5-F-3-pyridinyl, R³ is MeS and R¹ is Me. |
| 286F | Q³ is 5-F-3-pyridinyl, R³ is Et and R¹ is Me. |
| 287F | Q³ is 5-F-3-pyridinyl, R³ is Me and R¹ is Et. |
| 288F | Q³ is 5-F-3-pyridinyl, R³ is Cl and R¹ is Et. |
| 289F | Q³ is 6-Cl-3-pyridazinyl, R³ is Cl and R¹ is CFH₂. |
| 290F | Q³ is 6-Cl-3-pyridazinyl, R³ is Br and R¹ is Me. |
| 291F | Q³ is 6-Cl-3-pyridazinyl, R³ is I and R¹ is Me. |
| 292F | Q³ is 6-Cl-3-pyridazinyl, R³ is Me and R¹ is Me. |
| 293F | Q³ is 6-Cl-3-pyridazinyl, R³ is MeO and R¹ is Me. |
| 294F | Q³ is 6-Cl-3-pyridazinyl, R³ is MeS and R¹ is Me. |
| 295F | Q³ is 6-Cl-3-pyridazinyl, R³ is Et and R¹ is Me. |
| 296F | Q³ is 6-Cl-3-pyridazinyl, R³ is Me and R¹ is Et. |
| 297F | Q³ is 6-Cl-3-pyridazinyl, R³ is Cl and R¹ is Et. |
| 298F | Q³ is 5-Cl-3-pyridazinyl, R³ is Cl and R¹ is CFH₂. |
| 299F | Q³ is 5-Cl-3-pyridazinyl, R³ is Br and R¹ is Me. |
| 300F | Q³ is 5-Cl-3-pyridazinyl, R³ is I and R¹ is Me. |
| 301F | Q³ is 5-Cl-3-pyridazinyl, R³ is Me and R¹ is Me. |
| 302F | Q³ is 5-Cl-3-pyridazinyl, R³ is MeO and R¹ is Me. |
| 303F | Q³ is 5-Cl-3-pyridazinyl, R³ is MeS and R¹ is Me. |
| 304F | Q³ is 5-Cl-3-pyridazinyl, R³ is Et and R¹ is Me. |
| 305F | Q³ is 5-Cl-3-pyridazinyl, R³ is Me and R¹ is Et. |
| 306F | Q³ is 5-Cl-3-pyridazinyl, R³ is Cl and R¹ is Et. |
| 307F | Q³ is 2-Cl-5-pyrimidinyl, R³ is Cl and R¹ is CFH₂. |
| 308F | Q³ is 2-Cl-5-pyrimidinyl, R³ is Br and R¹ is Me. |
| 309F | Q³ is 2-Cl-5-pyrimidinyl, R³ is I and R¹ is Me. |
| 310F | Q³ is 2-Cl-5-pyrimidinyl, R³ is Me and R¹ is Me. |
| 311F | Q³ is 2-Cl-5-pyrimidinyl, R³ is MeO and R¹ is Me. |
| 312F | Q³ is 2-Cl-5-pyrimidinyl, R³ is MeS and R¹ is Me. |
| 313F | Q³ is 2-Cl-5-pyrimidinyl, R³ is Et and R¹ is Me. |
| 314F | Q³ is 2-Cl-5-pyrimidinyl, R³ is Me and R¹ is Et. |
| 315F | Q³ is 2-Cl-5-pyrimidinyl, R³ is Cl and R¹ is Et. |
| 316F | Q³ is 2-Cl-2-pyrimidinyl, R³ is Cl and R¹ is CFH₂. |
| 317F | Q³ is 2-Cl-2-pyrimidinyl, R³ is Br and R¹ is Me. |
| 318F | Q³ is 2-Cl-2-pyrimidinyl, R³ is I and R¹ is Me. |
| 319F | Q³ is 2-Cl-2-pyrimidinyl, R³ is Me and R¹ is Me. |
| 320F | Q³ is 2-Cl-2-pyrimidinyl, R³ is MeO and R¹ is Me. |
| 321F | Q³ is 2-Cl-2-pyrimidinyl, R³ is MeS and R¹ is Me. |
| 322F | Q³ is 2-Cl-2-pyrimidinyl, R³ is Et and R¹ is Me. |
| 323F | Q³ is 2-Cl-2-pyrimidinyl, R³ is Me and R¹ is Et. |
| 324F | Q³ is 2-Cl-2-pyrimidinyl, R³ is Cl and R¹ is Et. |
| 325F | Q³ is 5-Me-2-thienyl, R³ is Cl and R¹ is CFH₂. |
| 326F | Q³ is 5-Me-2-thienyl, R³ is Br and R¹ is Me. |
| 327F | Q³ is 5-Me-2-thienyl, R³ is I and R¹ is Me. |
| 328F | Q³ is 5-Me-2-thienyl, R³ is Me and R¹ is Me. |
| 329F | Q³ is 5-Me-2-thienyl, R³ is MeO and R¹ is Me. |
| 330F | Q³ is 5-Me-2-thienyl, R³ is MeS and R¹ is Me. |
| 331F | Q³ is 5-Me-2-thienyl, R³ is Et and R¹ is Me. |
| 332F | Q³ is 5-Me-2-thienyl, R³ is Me and R¹ is Et. |
| 333F | Q³ is 5-Me-2-thienyl, R³ is Cl and R¹ is Et. |
| 334F | Q³ is 5-Me-3-thienyl, R³ is Cl and R¹ is CFH₂. |
| 335F | Q³ is 5-Me-3-thienyl, R³ is Br and R¹ is Me. |
| 336F | Q³ is 5-Me-3-thienyl, R³ is I and R¹ is Me. |
| 337F | Q³ is 5-Me-3-thienyl, R³ is Me and R¹ is Me. |
| 338F | Q³ is 5-Me-3-thienyl, R³ is MeO and R¹ is Me. |
| 339F | Q³ is 5-Me-3-thienyl, R³ is MeS and R¹ is Me. |
| 340F | Q³ is 5-Me-3-thienyl, R³ is Et and R¹ is Me. |
| 341F | Q³ is 5-Me-3-thienyl, R³ is Me and R¹ is Et. |
| 342F | Q³ is 5-Me-3-thienyl, R³ is Cl and R¹ is Et. |
| 343F | Q³ is 1-Me-1H-pyrazol-3-yl, R³ is Cl and R¹ is CFH₂. |
| 344F | Q³ is 1-Me-1H-pyrazol-3-yl, R³ is Br and R¹ is Me. |
| 345F | Q³ is 1-Me-1H-pyrazol-3-yl, R³ is I and R¹ is Me. |
| 346F | Q³ is 1-Me-1H-pyrazol-3-yl, R³ is Me and R¹ is Me. |
| 347F | Q³ is 1-Me-1H-pyrazol-3-yl, R³ is MeO and R¹ is Me. |
| 348F | Q³ is 1-Me-1H-pyrazol-3-yl, R³ is MeS and R¹ is Me. |
| 349F | Q³ is 1-Me-1H-pyrazol-3-yl, R³ is Et and R¹ is Me. |
| 350F | Q³ is 1-Me-1H-pyrazol-3-yl,, R³ is Me and R¹ is Et. |
| 351F | Q³ is 1-Me-1H-pyrazol-3-yl, R³ is Cl and R¹ is Et. |
| 352F | Q³ is 1-Me-1H-pyrazol-4-yl, R³ is Cl and R¹ is CFH₂. |
| 353F | Q³ is 1-Me-1H-pyrazol-4-yl, R³ is Br and R¹ is Me. |
| 354F | Q³ is 1-Me-1H-pyrazol-4-yl, R³ is I and R¹ is Me. |
| 355F | Q³ is 1-Me-1H-pyrazol-4-yl, R³ is Me and R¹ is Me. |
| 356F | Q³ is 1-Me-1H-pyrazol-4-yl, R³ is MeO and R¹ is Me. |
| 357F | Q³ is 1-Me-1H-pyrazol-4-yl, R³ is MeS and R¹ is Me. |
| 358F | Q³ is 1-Me-1H-pyrazol-4-yl, R³ is Et and R¹ is Me. |
| 359F | Q³ is 1-Me-1H-pyrazol-4-yl, R³ is Me and R¹ is Et. |
| 360F | Q³ is 1-Me-1H-pyrazol-4-yl, R³ is Cl and R¹ is Et. |
| 361F | Q³ is 2-Me-5-thiazolyl, R³ is Cl and R¹ is CFH₂. |
| 362F | Q³ is 2-Me-5-thiazolyl, R³ is Br and R¹ is Me. |
| 363F | Q³ is 2-Me-5-thiazolyl, R³ is I and R¹ is Me. |
| 364F | Q³ is 2-Me-5-thiazolyl, R³ is Me and R¹ is Me. |
| 365F | Q³ is 2-Me-5-thiazolyl, R³ is MeO and R¹ is Me. |
| 366F | Q³ is 2-Me-5-thiazolyl, R³ is MeS and R¹ is Me. |
| 367F | Q³ is 2-Me-5-thiazolyl, R³ is Et and R¹ is Me. |
| 368F | Q³ is 2-Me-5-thiazolyl, R³ is Me and R¹ is Et. |
| 369F | Q³ is 2-Me-5-thiazolyl, R³ is Cl and R¹ is Et. |
| 370F | Q³ is 2-Cl-5-thiazolyl, R³ is Cl and R¹ is CFH₂. |
| 371F | Q³ is 2-Cl-5-thiazolyl, R³ is Br and R¹ is Me. |
| 372F | Q³ is 2-Cl-5-thiazolyl, R³ is I and R¹ is Me. |
| 373F | Q³ is 2-Cl-5-thiazolyl, R³ is Me and R¹ is Me. |
| 374F | Q³ is 2-Cl-5-thiazolyl, R³ is MeO and R¹ is Me. |
| 375F | Q³ is 2-Cl-5-thiazolyl, R³ is MeS and R¹ is Me. |
| 376F | Q³ is 2-Cl-5-thiazolyl, R³ is Et and R¹ is Me. |
| 377F | Q³ is 2-Cl-5-thiazolyl, R³ is Me and R¹ is Et. |
| 378F | Q³ is 2-Cl-5-thiazolyl, R³ is Cl and R¹ is Et. |
| 379F | Q³ is 5-Me-3-isothiazolyl, R³ is Cl and R¹ is CFH₂. |
| 380F | Q³ is 5-Me-3-isothiazolyl, R³ is Br and R¹ is Me. |
| 381F | Q³ is 5-Me-3-isothiazolyl, R³ is I and R¹ is Me. |
| 382F | Q³ is 5-Me-3-isothiazolyl, R³ is Me and R¹ is Me. |
| 383F | Q³ is 5-Me-3-isothiazolyl, R³ is MeO and R¹ is Me. |
| 384F | Q³ is 5-Me-3-isothiazolyl, R³ is MeS and R¹ is Me. |
| 385F | Q³ is 5-Me-3-isothiazolyl, R³ is Et and R¹ is Me. |
| 386F | Q³ is 5-Me-3-isothiazolyl, R³ is Me and R¹ is Et. |
| 387F | Q³ is 5-Me-3-isothiazolyl, R³ is Cl and R¹ is Et. |

TABLE 7

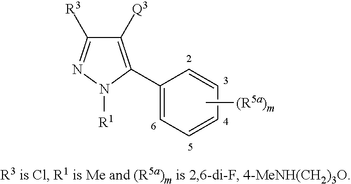

| Q³ | Q³ | Q³ | Q³ | Q³ |
|---|---|---|---|---|
| 4-Cl—Ph | 3-F—Ph | 3,4-di-F—Ph | 6-CF₃-3-pyridinyl | 5-Me-3-pyridinyl |
| 4-F—Ph | 3-Br—Ph | 3,4-di-Cl—Ph | 2-Cl, 6-CF₃-3-pyridinyl | 5-F-3-pyridinyl |
| 4-Br—Ph | 3-Me—Ph | 2-Cl, 3,5-di-MeO—Ph | 2-Cl, 6-MeO-3-pyridinyl | 6-Me-3-pyridinyl |
| 4-Me—Ph | 3-Et—Ph | 4-Cl, 3,5-di-MeO—Ph | 2-Cl, 6-Me-3-pyridinyl | 6-Cl-3-pyridinyl |
| 4-Et—Ph | 3-F, 4-Me—Ph | 5-MeO-3-pyridinyl | 6-MeO-3-pyridinyl | 5-Cl-3-pyridinyl |
| 4-Cl—Bn | 4-Cl, 3-F—Ph | 6-MeO-3-pyridinyl | 6-Br-3-pyridinyl | 6-CF₃-3-pyridinyl |
| 4-F—Bn | 2-Cl, 4-F—Ph | 2-Me-5-pyridinyl | 2-CF₃-5-pyridinyl | 2-Cl-5-pyridinyl |
| 3-Cl—Ph | 3,5-di-MeO—Ph | 6-F-3-pyridinyl | 6-Me-3-pyridinyl | 6-Cl-3-pyridinyl |
| | | | | 2-MeO-5-pyridinyl |

The present disclosure also includes Tables 1G through 23G, each of which is constructed the same as Table 7 above except that the row heading in Table 7 (i.e. “R³ is Cl, R¹ is Me and (R^{5a})ₘ is 2,6-di-F, 4-MeNH(CH₂)₃O”) is replaced with the respective row heading shown below. Thus, for example, in Table 1G the row heading is “R³ is Br, R¹ is Me and (R^{5a})ₘ is 2,6-di-F, 4-MeNH(CH₂)₃O”, and Q³ is as defined in Table 7 above. Tables 2G through 23G are constructed similarly.

| Table | |
|---|---|
| Number | Row Heading |
| 1G | R³ is Br, R¹ is Me and (R^{5a})ₘ is 2,6-di-F, 4-MeNH(CH₂)₃O. |
| 2G | R³ is Me, R¹ is Me and (R^{5a})ₘ is 2,6-di-F, 4-MeNH(CH₂)₃O. |
| 3G | R³ is Cl, R¹ is Me and (R^{5b})ₘ is 2,6-di-F, 4-Me₂N(CH₂)₃O. |
| 4G | R³ is Br, R¹ is Me and (R^{5b})ₘ is 2,6-di-F, 4-Me₂N(CH₂)₃O. |
| 5G | R³ is Me, R¹ is Me and (R^{5b})ₘ is 2,6-di-F, 4-Me₂N(CH₂)₃O. |
| 6G | R³ is Cl, R¹ is Me and (R^{5a})ₘ is 2,6-di-F, 4-MeO(CH₂)₃O. |
| 7G | R³ is Br, R¹ is Me and (R^{5a})ₘ is 2,6-di-F, 4-MeO(CH₂)₃O. |
| 8G | R³ is Me, R¹ is Me and (R^{5a})ₘ is 2,6-di-F, 4-MeO(CH₂)₃O. |
| 9G | R³ is Cl, R¹ is Me and (R^{5a})ₘ is 2-Cl-6-F, 4-MeNH(CH₂)₃O. |
| 10G | R³ is Br, R¹ is Me and (R^{5a})ₘ is 2-Cl-6-F, 4-MeNH(CH₂)₃O. |
| 11G | R³ is Me, R¹ is Me and (R^{5a})ₘ is 2-Cl-6-F, 4-MeNH(CH₂)₃O. |
| 12G | R³ is Cl, R¹ is Me and (R^{5a})ₘ is 2,6-di-F, 3-MeNH(CH₂)₃O. |
| 13G | R³ is Br, R¹ is Me and (R^{5a})ₘ is 2,6-di-F, 3-MeNH(CH₂)₃O. |
| 14G | R³ is Me, R¹ is Me and (R^{5a})ₘ is 2,6-di-F, 3-MeNH(CH₂)₃O. |
| 15G | R³ is Cl, R¹ is Me and (R^{5a})ₘ is 2,6-di-F, 3-Me₂N(CH₂)₃O. |
| 16G | R³ is Br, R¹ is Me and (R^{5a})ₘ is 2,6-di-F, 3-Me₂N(CH₂)₃O. |
| 17G | R³ is Me, R¹ is Me and (R^{5a})ₘ is 2,6-di-F, 3-Me₂N(CH₂)₃O. |
| 18G | R³ is Cl, R¹ is Me and (R^{5a})ₘ is 2,6-di-F, 3-MeO(CH₂)₃O. |
| 19G | R³ is Br, R¹ is Me and (R^{5a})ₘ is 2,6-di-F, 3-MeO(CH₂)₃O. |
| 20G | R³ is Me, R¹ is Me and (R^{5a})ₘ is 2,6-di-F, 3-MeO(CH₂)₃O. |
| 21G | R³ is Cl, R¹ is Me and (R^{5a})ₘ is 2-Cl-6-F, 4-MeNH(CH₂)₃O. |
| 22G | R³ is Br, R¹ is Me and (R^{5a})ₘ is 2-Cl-6-F, 3-MeNH(CH₂)₃O. |
| 23G | R³ is Me, R¹ is Me and (R^{5a})ₘ is 2-Cl-6-F, 3-MeNH(CH₂)₃O. |

TABLE 8

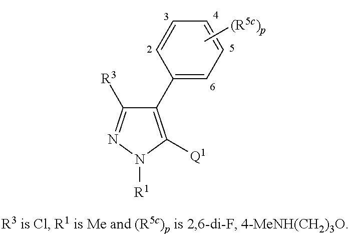

| Q¹ | Q¹ | Q¹ | Q¹ | Q¹ |
|---|---|---|---|---|
| 4-Cl—Ph | 3-F—Ph | 3,4-di-F—Ph | 6-CF₃-3-pyridinyl | 5-Me-3-pyridinyl |
| 4-F—Ph | 3-Br—Ph | 3,4-di-Cl—Ph | 2-Cl, 6-CF₃-3-pyridinyl | 5-F-3-pyridinyl |
| 4-Br—Ph | 3-Me—Ph | 2-Cl, 3,5-di-MeO—Ph | 2-Cl, 6-MeO-3-pyridinyl | 6-Me-3-pyridinyl |
| 4-Me—Ph | 3-Et—Ph | 4-Cl, 3,5-di-MeO—Ph | 2-Cl, 6-Me-3-pyridinyl | 6-Cl-3-pyridinyl |
| 4-Et—Ph | 3-F, 4-Me—Ph | 5-MeO-3-pyridinyl | 6-MeO-3-pyridinyl | 5-Cl-3-pyridinyl |
| 4-Cl—Bn | 4-Cl, 3-F—Ph | 6-MeO-3-pyridinyl | 6-Br-3-pyridinyl | 6-CF₃-3-pyridinyl |
| 4-F—Bn | 2-Cl, 4-F—Ph | 2-Me-5-pyridinyl | 2-CF₃-5-pyridinyl | 2-Cl-5-pyridinyl |
| 3-Cl—Ph | 3,5-di-MeO—Ph | 6-F-3-pyridinyl | 6-Me-3-pyridinyl | 6-Cl-3-pyridinyl |
| | | | | 2-MeO-5-pyridinyl |

The present disclosure also includes Tables 1H through 23H, each of which is constructed the same as Table 8 above except that the row heading in Table 8 (i.e. “R³ is Cl, R¹ is Me and (R^{5c})ₚ is 2,6-di-F, 4-MeNH(CH₂)₃O”) is replaced with the respective row heading shown below. Thus, for example, in Table 1H the row heading is “R³ is Br, R¹ is Me and (R^{5c})ₚ is 2,6-di-F, 4-MeNH(CH₂)₃O”, and Q¹ is as defined in Table 8 above. Tables 2G through 23G are constructed similarly.

| Table | |
|---|---|
| Number | Row Heading |
| 1H | R³ is Br, R¹ is Me and (R^{5c})ₚ is 2,6-di-F, 4-MeNH(CH₂)₃O. |
| 2H | R³ is Me, R¹ is Me and (R^{5c})ₚ is 2,6-di-F, 4-MeNH(CH₂)₃O. |
| 3H | R³ is Cl, R¹ is Me and (R^{5c})ₚ is 2,6-di-F, 4-Me₂N(CH₂)₃O. |
| 4H | R³ is Br, R¹ is Me and (R^{5c})ₚ is 2,6-di-F, 4-Me₂N(CH₂)₃O. |
| 5H | R³ is Me, R¹ is Me and (R^{5c})ₚ is 2,6-di-F, 4-Me₂N(CH₂)₃O. |
| 6H | R³ is Cl, R¹ is Me and (R^{5c})ₚ is 2,6-di-F, 4-MeO(CH₂)₃O. |
| 7H | R³ is Br, R¹ is Me and (R^{5a})ₚ is 2,6-di-F, 4-MeO(CH₂)₃O. |
| 8H | R³ is Me, R¹ is Me and (R^{5c})ₚ is 2,6-di-F, 4-MeO(CH₂)₃O. |
| 9H | R³ is Cl, R¹ is Me and (R^{5c})ₚ is 2-Cl-6-F, 4-MeNH(CH₂)₃O. |
| 10H | R³ is Br, R¹ is Me and (R^{5c})ₚ is 2-Cl-6-F, 4-MeNH(CH₂)₃O. |
| 11H | R³ is Me, R¹ is Me and (R^{5c})ₚ is 2-Cl-6-F, 4-MeNH(CH₂)₃O. |
| 12H | R³ is Cl, R¹ is Me and (R^{5c})ₚ is 2,6-di-F, 3-MeNH(CH₂)₃O. |
| 13H | R³ is Br, R¹ is Me and (R^{5c})ₚ is 2,6-di-F, 3-MeNH(CH₂)₃O. |
| 14H | R³ is Me, R¹ is Me and (R^{5c})ₚ is 2,6-di-F, 3-MeNH(CH₂)₃O. |
| 15H | R³ is Cl, R¹ is Me and (R^{5c})ₚ is 2,6-di-F, 3-Me₂N(CH₂)₃O. |
| 16H | R³ is Br, R¹ is Me and (R^{5c})ₚ is 2,6-di-F, 3-Me₂N(CH₂)₃O. |
| 17H | R³ is Me, R¹ is Me and (R^{5c})ₚ is 2,6-di-F, 3-Me₂N(CH₂)₃O. |
| 18H | R³ is Cl, R¹ is Me and (R^{5c})ₚ is 2,6-di-F, 3-MeO(CH₂)₃O. |
| 19H | R³ is Br, R¹ is Me and (R^{5c})ₚ is 2,6-di-F, 3-MeO(CH₂)₃O. |
| 20H | R³ is Me, R¹ is Me and (R^{5c})ₚ is 2,6-di-F, 3-MeO(CH₂)₃O. |
| 21H | R³ is Cl, R¹ is Me and (R^{5c})ₚ is 2-Cl-6-F, 4-MeNH(CH₂)₃O. |
| 22H | R³ is Br, R¹ is Me and (R^{5c})ₚ is 2-Cl-6-F, 3-MeNH(CH₂)₃O. |
| 23H | R³ is Me, R¹ is Me and (R^{5c})ₚ is 2-Cl-6-F, 3-MeNH(CH₂)₃O. |

TABLE 9

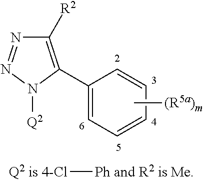

| (R^{5a})ₘ | (R^{5a})ₘ | (R^{5a})ₘ | (R^{5a})ₘ | (R^{5a})ₘ |
|---|---|---|---|---|
| 2,6-di-F | 2,4-di-Cl | 4-CN, 2,6-di-F | 2-CF₃, 4-F | 2-Cl, 4-NO₂ |
| 2,4,6-tri-F | 2-Cl, 4,6-di-F | 2,6-di-F, 4-Me | 2-CF₂HO, 4-F | 2-NO₂, 4-F |
| 2,3,6-tri-F | 2-Cl, 6-F | 2-Cl, 5-CF₃ | 2-CN, 6-F | 2,3,4,5,6-penta-F |
| 2,4,5-tri-F | 2-Br, 6-F | 2-Cl, 4-Me | 2,5-di-Cl | 2,5-di-Cl, 4-F |
| 2,3,4-tri-F | 2-F, 6-CF₃ | 2-Cl, 4-MeO | 2-CF₃, 4-MeO | 2,3-di-Cl, 4-F |
| 2-Cl, 4-F | 2-F, 6-CF₂HO | 2-Br, 4-MeO | 2-F, 6-Me | 2-Cl, 5-CN |
| 2-Br, 4-F | 2-I, 4-F | 2,6-di-F, 3-Cl | 2,6-di-F, 3-Me | 2,4-di-F, 5-CN |
| 2,4-di-F | 4-Cl, 2,6-di-F | 2,6-di-F, 3-CN | 2-CF₃ | 2-Cl, 6-F, 3-MeO |
| 2,6-di-Cl | 2,6-di-F, 4-MeO | 2,6-di-F, 3-MeO | 2-CF₃O | |
| 2,6-di-F, 4-EtO | 2-Cl, 6-F, 4-MeO | 2,6-di-F, 4-CF₂HO | 2,6-di-F, 4-NO₂ | |
| 2-Cl, 3,6-di-F | 2-Cl, 6-F, 5-MeO | 2,6-di-F, 3-CF₂HO | 2,6-di-F, 3-EtO | |

The present disclosure also includes Tables 1J through 65J, each of which are constructed the same as Table 9 above except that the row heading in Table 9 (i.e. “Q² is 4-Cl-Ph and R² is Me”) is replaced with the respective row headings shown below. Thus, for example, in Table 1J the row heading is “Q² is 4-Cl-Ph and R² is Br”, and (R^{5a})ₘ is as defined in Table 9 above. Tables 2J through 65J are constructed similarly.

| Table Number | Row Heading |
|---|---|
| 1J | Q² is 4-Cl-Ph, R² is Br. |
| 2J | Q² is 4-Cl-Ph, R² is Cl. |
| 3J | Q² is 3-F-Ph, R² is Me. |
| 4J | Q² is 3-F-Ph, R² is Br. |
| 5J | Q² is 3-F-Ph, R² is Cl. |
| 6J | Q² is 3-OCF₂H-Ph, R² is Me. |
| 7J | Q² is 3-CF₂HO-Ph, R² is Br. |
| 8J | Q² is 3-CF₂HO-Ph, R² is Cl. |
| 9J | Q² is 4-Cl, 3-F-Ph, R² is Me. |
| 10J | Q² is 4-Cl, 3-F-Ph, R² is Br. |
| 11J | Q² is 4-Cl, 3-F-Ph, R² is Cl. |
| 12J | Q² is 2-Cl, 4-F-Ph, R² is Me. |
| 13J | Q² is 2-Cl, 4-F-Ph, R² is Br. |
| 14J | Q² is 2-Cl, 4-F-Ph, R² is Cl. |
| 15J | Q² is 3,5-di-MeO-Ph, R² is Me. |
| 16J | Q² is 3,5-di-MeO-Ph, R² is Br. |
| 17J | Q² is 3,5-di-MeO-Ph, R² is Cl. |
| 18J | Q² is 2-Cl, 3,5-di-MeO-Ph, R² is Me. |
| 19J | Q² is 2-Cl, 3,5-di-MeO-Ph, R² is Br. |
| 20J | Q² is 2-Cl, 3,5-di-MeO-Ph, R² is Cl. |
| 21J | Q² is 4-Cl-Bn, R² is Me. |
| 22J | Q² is 4-Cl-Bn, R² is Br. |
| 23J | Q² is 4-Cl-Bn, R² is Cl. |
| 24J | Q² is 6-Cl-3-pyridinyl, R² is Me. |
| 25J | Q² is 6-Cl-3-pyridinyl, R² is Br. |
| 26J | Q² is 6-Cl-3-pyridinyl, R² is Cl. |
| 27J | Q² is 6-MeO-3-pyridinyl, R² is Me. |
| 28J | Q² is 6-MeO-3-pyridinyl, R² is Br. |
| 29J | Q² is 6-MeO-3-pyridinyl, R² is Cl. |
| 30J | Q² is 5-F-3-pyridinyl, R² is Me. |
| 31J | Q² is 5-F-3-pyridinyl, R² is Br. |
| 32J | Q² is 5-F-3-pyridinyl, R² is Cl. |
| 33J | Q² is 6-Cl-3-pyridazinyl, R² is Me. |
| 34J | Q² is 6-Cl-3-pyridazinyl, R² is Br. |
| 35J | Q² is 6-Cl-3-pyridazinyl, R² is Cl. |
| 36J | Q² is 5-Cl-3-pyridazinyl, R² is Me. |
| 37J | Q² is 5-Cl-3-pyridazinyl, R² is Br. |
| 38J | Q² is 5-Cl-3-pyridazinyl, R² is Cl. |
| 39J | Q² is 2-Cl-5-pyrimidinyl, R² is Me. |
| 40J | Q² is 2-Cl-5-pyrimidinyl, R² is Br. |
| 41J | Q² is 2-Cl-5-pyrimidinyl, R² is Cl. |
| 42J | Q² is 2-Me-5-pyrimidinyl, R² is Me. |
| 43J | Q² is 2-Me-5-pyrimidinyl, R² is Br. |
| 44J | Q² is 2-Me-5-pyrimidinyl, R² is Cl. |
| 45J | Q² is 5-Cl-2-pyrimidinyl, R² is Me. |
| 46J | Q² is 5-Cl-2-pyrimidinyl, R² is Br. |
| 47J | Q² is 5-Cl-2-pyrimidinyl, R² is Cl. |
| 48J | Q² is 5-Cl-2-thienyl, R² is Me. |
| 49J | Q² is 5-Cl-2-thienyl, R² is Br. |
| 50J | Q² is 5-Cl-2-thienyl, R² is Cl. |
| 51J | Q² is 5-Me-3-thienyl, R² is Me. |
| 52J | Q² is 5-Me-3-thienyl, R² is Br. |
| 53J | Q² is 5-Me-3-thienyl, R² is Cl. |
| 54J | Q² is 1-Me-1H-pyrazol-3-yl, R² is Me. |
| 55J | Q² is 1-Me-1H-pyrazol-3-yl, R² is Br. |
| 56J | Q² is 1-Me-1H-pyrazol-3-yl, R² is Cl. |
| 57J | Q² is 1-Me-1H-pyrazol-4-yl, R² is Me. |
| 58J | Q² is 1-Me-1H-pyrazol-4-yl, R² is Br. |
| 59J | Q² is 1-Me-1H-pyrazol-4-yl, R² is Cl. |
| 60J | Q² is 2-Cl-5-thiazolyl, R² is Me. |
| 61J | Q² is 2-Cl-5-thiazolyl, R² is Br. |
| 62J | Q² is 2-Cl-5-thiazolyl, R² is Cl. |
| 63J | Q² is 5-Me-3-isothiazolyl, R² is Me. |
| 64J | Q² is 5-Me-3-isothiazolyl, R² is Br. |
| 65J | Q² is 5-Me-3-isothiazolyl, R² is Cl. |

TABLE 10

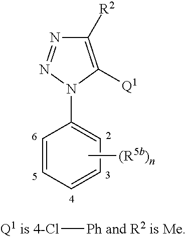

| (R^{5b})ₙ | (R^{5b})ₙ | (R^{5b})ₙ | (R^{5b})ₙ | (R^{5b})ₙ |
|---|---|---|---|---|
| 2,6-di-F | 2,4-di-Cl | 4-CN, 2,6-di-F | 2-CF₃, 4-F | 2-Cl, 4-NO₂ |
| 2,4,6-tri-F | 2-Cl, 4,6-di-F | 2,6-di-F, 4-Me | 2-CF₂HO, 4-F | 2-NO₂, 4-F |
| 2,3,6-tri-F | 2-Cl, 6-F | 2-Cl, 5-CF₃ | 2-CN, 6-F | 2,3,4,5,6-penta-F |
| 2,4,5-tri-F | 2-Br, 6-F | 2-Cl, 4-Me | 2,5-di-Cl | 2,5-di-Cl, 4-F |
| 2,3,4-tri-F | 2-F, 6-CF₃ | 2-Cl, 4-MeO | 2-CF₃, 4-MeO | 2,3-di-Cl, 4-F |
| 2-Cl, 4-F | 2-F, 6-CF₂HO | 2-Br, 4-MeO | 2-F, 6-Me | 2-Cl, 5-CN |
| 2-Br, 4-F | 2-I, 4-F | 2,6-di-F, 3-Cl | 2,6-di-F, 3-Me | 2,4-di-F, 5-CN |
| 2,4-di-F | 4-Cl, 2,6-di-F | 2,6-di-F, 3-CN | 2-CF₃ | 2-Cl, 6-F, 3-MeO |
| 2,6-di-Cl | 2,6-di-F, 4-MeO | 2,6-di-F, 3-MeO | 2-CF₃O | |
| 2,6-di-F, 4-EtO | 2-Cl, 6-F, 4-MeO | 2,6-di-F, 4-CF₂HO | 2,6-di-F, 4-NO₂ | |
| 2-Cl, 3,6-di-F | 2-Cl, 6-F, 5-MeO | 2,6-di-F, 3-CF₂HO | 2,6-di-F, 3-EtO | |

The present disclosure also includes Tables 1K through 65K, each of which are constructed the same as Table 10 above except that the row heading in Table 10 (i.e. “Q¹ is 4-Cl-Ph and R² is Me”) is replaced with the respective row heading shown below. Thus, for example, in Table 1K the row heading is “Q¹ is 4-Cl-Ph and R¹ is Br”, and (R^{5b})ₙ is as defined in Table 10 above. Tables 2K through 65K are constructed similarly.

| Table Number | Row Heading |
|---|---|
| 1K | Q¹ is 4-Cl-Ph, R² is Br. |
| 2K | Q¹ is 4-Cl-Ph, R² is Cl. |
| 3K | Q¹ is 3-F-Ph, R² is Me. |
| 4K | Q¹ is 3-F-Ph, R² is Br. |
| 5K | Q¹ is 3-F-Ph, R² is Cl. |
| 6K | Q¹ is 3-CF₂HO-Ph, R² is Me. |
| 7K | Q¹ is 3-CF₂HO-Ph, R² is Br. |
| 8K | Q¹ is 3-CF₂HO-Ph, R² is Cl. |
| 9K | Q¹ is 4-Cl, 3-F-Ph, R² is Me. |
| 10K | Q¹ is 4-Cl, 3-F-Ph, R² is Br. |
| 11K | Q¹ is 4-Cl, 3-F-Ph, R² is Cl. |
| 12K | Q¹ is 2-Cl, 4-F-Ph, R² is Me. |
| 13K | Q¹ is 2-Cl, 4-F-Ph, R² is Br. |
| 14K | Q¹ is 2-Cl, 4-F-Ph, R² is Cl. |
| 15K | Q¹ is 3,5-di-MeO-Ph, R² is Me. |
| 16K | Q¹ is 3,5-di-MeO-Ph, R² is Br. |
| 17K | Q¹ is 3,5-di-MeO-Ph, R² is Cl. |
| 18K | Q¹ is 2-Cl, 3,5-di-MeO-Ph, R² is Me. |
| 19K | Q¹ is 2-Cl, 3,5-di-MeO-Ph, R² is Br. |
| 20K | Q¹ is 2-Cl, 3,5-di-MeO-Ph, R² is Cl. |
| 21K | Q¹ is 4-Cl-Bn, R² is Me. |
| 22K | Q¹ is 4-Cl-Bn, R² is Br. |
| 23K | Q¹ is 4-Cl-Bn, R² is Cl. |
| 24K | Q¹ is 6-Cl-3-pyridinyl, R² is Me. |
| 25K | Q¹ is 6-Cl-3-pyridinyl, R² is Br. |
| 26K | Q¹ is 6-Cl-3-pyridinyl, R² is Cl. |
| 27K | Q¹ is 6-MeO-3-pyridinyl, R² is Me. |
| 28K | Q¹ is 6-MeO-3-pyridinyl, R² is Br. |
| 29K | Q¹ is 6-MeO-3-pyridinyl, R² is Cl. |
| 30K | Q¹ is 5-F-3-pyridinyl, R² is Me. |
| 31K | Q¹ is 5-F-3-pyridinyl, R² is Br. |
| 32K | Q¹ is 5-F-3-pyridinyl, R² is Cl. |
| 33K | Q¹ is 6-Cl-3-pyridazinyl, R² is Me. |
| 34K | Q¹ is 6-Cl-3-pyridazinyl, R² is Br. |
| 35K | Q¹ is 6-Cl-3-pyridazinyl, R² is Cl. |
| 36K | Q¹ is 5-Cl-3-pyridazinyl, R² is Me. |
| 37K | Q¹ is 5-Cl-3-pyridazinyl, R² is Br. |
| 38K | Q¹ is 5-Cl-3-pyridazinyl, R² is Cl. |
| 39K | Q¹ is 2-Cl-5-pyrimidinyl, R² is Me. |
| 40K | Q¹ is 2-Cl-5-pyrimidinyl, R² is Br. |
| 41K | Q¹ is 2-Cl-5-pyrimidinyl, R² is Cl. |
| 42K | Q¹ is 2-Me-5-pyrimidinyl, R² is Me. |
| 43K | Q¹ is 2-Me-5-pyrimidinyl, R² is Br. |
| 44K | Q¹ is 2-Me-5-pyrimidinyl, R² is Cl. |
| 45K | Q¹ is 5-Cl-2-pyrimidinyl, R² is Me. |
| 46K | Q¹ is 5-Cl-2-pyrimidinyl, R² is Br. |
| 47K | Q¹ is 5-Cl-2-pyrimidinyl, R² is Cl. |
| 48K | Q¹ is 5-Cl-2-thienyl, R² is Me. |
| 49K | Q¹ is 5-Cl-2-thienyl, R² is Br. |
| 50K | Q¹ is 5-Cl-2-thienyl, R² is Cl. |
| 51K | Q¹ is 5-Me-3-thienyl, R² is Me. |
| 52K | Q¹ is 5-Me-3-thienyl, R² is Br. |
| 53K | Q¹ is 5-Me-3-thienyl, R² is Cl. |
| 54K | Q¹ is 1-Me-1H-pyrazol-3-yl, R² is Me. |
| 55K | Q¹ is 1-Me-1H-pyrazol-3-yl, R² is Br. |
| 56K | Q¹ is 1-Me-1H-pyrazol-3-yl, R² is Cl. |
| 57K | Q¹ is 1-Me-1H-pyrazol-4-yl, R² is Me. |
| 58K | Q¹ is 1-Me-1H-pyrazol-4-yl, R² is Br. |
| 59K | Q¹ is 1-Me-1H-pyrazol-4-yl, R² is Cl. |
| 60K | Q¹ is 2-Cl-5-thiazolyl, R² is Me. |
| 61K | Q¹ is 2-Cl-5-thiazolyl, R² is Br. |
| 62K | Q¹ is 2-Cl-5-thiazolyl, R² is Cl. |
| 63K | Q¹ is 5-Me-3-isothiazolyl, R² is Me. |
| 64K | Q¹ is 5-Me-3-isothiazolyl, R² is Br. |
| 65K | Q¹ is 5-Me-3-isothiazolyl, R² is Cl. |

TABLE 11

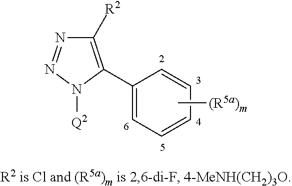

| Q² | Q² | Q² | Q² | Q² |
|---|---|---|---|---|
| 4-Cl—Ph | 3-F—Ph | 3,4-di-F—Ph | 6-CF₃-3-pyridinyl | 5-Me-3-pyridinyl |
| 4-F—Ph | 3-Br—Ph | 3,4-di-Cl—Ph | 2-Cl, 6-CF₃-3-pyridinyl | 5-F-3-pyridinyl |
| 4-Br—Ph | 3-Me—Ph | 2-Cl, 3,5-di-MeO—Ph | 2-Cl, 6-MeO-3-pyridinyl | 6-Me-3-pyridinyl |
| 4-Me—Ph | 3-Et—Ph | 4-Cl, 3,5-di-MeO—Ph | 2-Cl, 6-Me-3-pyridinyl | 6-Cl-3-pyridinyl |
| 4-Et—Ph | 3-F, 4-Me—Ph | 5-MeO-3-pyridinyl | 6-MeO-3-pyridinyl | 5-Cl-3-pyridinyl |
| 4-Cl—Bn | 4-Cl, 3-F—Ph | 6-MeO-3-pyridinyl | 6-Br-3-pyridinyl | 6-CF₃-3-pyridinyl |
| 4-F—Bn | 2-Cl, 4-F—Ph | 2-Me-5-pyridinyl | 2-CF₃-5-pyridinyl | 2-Cl-5-pyridinyl |
| 3-Cl—Ph | 3,5-di-MeO—Ph | 6-F-3-pyridinyl | 6-Me-3-pyridinyl | 6-Cl-3-pyridinyl |
| | | | | 2-MeO-5-pyridinyl |

The present disclosure also includes Tables 1L through 17L, each of which are constructed the same as Table 11 above except that the row heading in Table 11 (i.e. “R² is Cl and (R^{5a})ₘ is 2,6-di-F, 4-MeNH(CH₂)₃O.”) is replaced with the respective row heading shown below. Thus, for example, in Table 1L the row heading is “R² is Br and (R^{5a})ₘ is 2,6-di-F, 4-MeNH(CH₂)₃O”, and Q² is as define in Table 11 above. Tables 2L through 17L are constructed similarly.

| Table Number | Row Heading |
|---|---|
| 1L | R² is Br and (R^{5a})ₘ is 2,6-di-F, 4-MeNH(CH₂)₃O. |
| 2L | R² is Me and (R^{5a})ₘ is 2,6-di-F, 4-MeNH(CH₂)₃O. |
| 3L | R² is Cl and (R^{5a})ₘ is 2,6-di-F, 4-Me₂N(CH₂)₃O. |
| 4L | R² is Br and (R^{5a})ₘ is 2,6-di-F, 4-Me₂N(CH₂)₃O. |
| 5L | R² is Me and (R^{5a})ₘ is 2,6-di-F, 4-Me₂N(CH₂)₃O. |
| 6L | R² is Cl and (R^{5a})ₘ is 2,6-di-F, 4-MeO(CH₂)₃O. |
| 7L | R² is Br and (R^{5a})ₘ is 2,6-di-F, 4-MeO(CH₂)₃O. |
| 8L | R² is Me and (R^{5a})ₘ is 2,6-di-F, 4-MeO(CH₂)₃O. |
| 9 | R² is Cl and (R^{5a})ₘ is 2,6-di-F, 4-MeNH(CH₂)₃O. |
| 10L | R² is Br and (R^{5a})ₘ is 2,6-di-F, 4-MeNH(CH₂)₃O. |
| 11L | R² is Me and (R^{5a})ₘ is 2,6-di-F, 4-MeNH(CH₂)₃O. |
| 12L | R² is Cl and (R^{5a})ₘ is 2,6-di-F, 4-Me₂N(CH₂)₃O. |
| 13L | R² is Br and (R^{5a})ₘ is 2,6-di-F, 4-Me₂N(CH₂)₃O. |
| 14L | R² is Me and (R^{5a})ₘ is 2,6-di-F, 4-Me₂N(CH₂)₃O. |
| 15L | R² is Cl and (R^{5a})ₘ is 2,6-di-F, 4-MeO(CH₂)₃O. |
| 16L | R² is Br and (R^{5a})ₘ is 2,6-di-F, 4-MeO(CH₂)₃O. |
| 17L | R² is Me and (R^{5a})ₘ is 2,6-di-F, 4-MeO(CH₂)₃O. |

TABLE 12

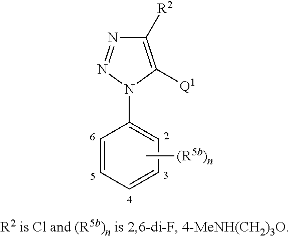

| Q¹ | Q¹ | Q¹ | Q¹ | Q¹ |
|---|---|---|---|---|
| 4-Cl—Ph | 3-F—Ph | 3,4-di-F—Ph | 6-CF₃-3-pyridinyl | 5-Me-3-pyridinyl |
| 4-F—Ph | 3-Br—Ph | 3,4-di-Cl—Ph | 2-Cl, 6-CF₃-3-pyridinyl | 5-F-3-pyridinyl |
| 4-Br—Ph | 3-Me—Ph | 2-Cl, 3,5-di-MeO—Ph | 2-Cl, 6-MeO-3-pyridinyl | 6-Me-3-pyridinyl |
| 4-Me—Ph | 3-Et—Ph | 4-Cl, 3,5-di-MeO—Ph | 2-Cl, 6-Me-3-pyridinyl | 6-Cl-3-pyridinyl |
| 4-Et—Ph | 3-F, 4-Me—Ph | 5-MeO-3-pyridinyl | 6-MeO-3-pyridinyl | 5-Cl-3-pyridinyl |
| 4-Cl—Bn | 4-Cl, 3-F—Ph | 6-MeO-3-pyridinyl | 6-Br-3-pyridinyl | 6-CF₃-3-pyridinyl |
| 4-F—Bn | 2-Cl, 4-F—Ph | 2-Me-5-pyridinyl | 2-CF₃-5-pyridinyl | 2-Cl-5-pyridinyl |
| 3-Cl—Ph | 3,5-di-MeO—Ph | 6-F-3-pyridinyl | 6-Me-3-pyridinyl | 6-Cl-3-pyridinyl |
| | | | | 2-MeO-5-pyridinyl |

The present disclosure also includes Tables 1M through 17M, each of which is constructed the same as Table 12 above except that the row heading in Table 12 (i.e. “R² is Cl and (R^{5b})ₙ is 2,6-di-F, 4-MeNH(CH₂)₃O.”) is replaced with the respective row heading shown below. Thus, for example, in Table 1M the row heading is “R² is Br and (R^{5b})ₙ is 2,6-di-F, 4-MeNH(CH₂)₃O”, and Q² is as defined in Table 12 above. Tables 2M through 17M are constructed similarly.

| Table Number | Row Heading |
|---|---|
| 1M | R² is Br and (R^{5b})ₙ is 2,6-di-F, 4-MeNH(CH₂)₃O. |
| 2M | R² is Me and (R^{5b})ₙ is 2,6-di-F, 4-MeNH(CH₂)₃O. |
| 3M | R² is Cl and (R^{5b})ₙ is 2,6-di-F, 4-Me₂N(CH₂)₃O. |
| 4M | R² is Br and (R^{5b})ₙ is 2,6-di-F, 4-Me₂N(CH₂)₃O. |
| 5M | R² is Me and (R^{5b})ₙ is 2,6-di-F, 4-Me₂N(CH₂)₃O. |
| 6M | R² is Cl and (R^{5b})ₙ is 2,6-di-F, 4-MeO(CH₂)₃O. |
| 7M | R² is Br and (R^{5b})ₙ is 2,6-di-F, 4-MeO(CH₂)₃O. |
| 8M | R² is Me and (R^{5b})ₙ is 2,6-di-F, 4-MeO(CH₂)₃O. |
| 9 | R² is Cl and (R^{5b})ₙ is 2,6-di-F, 4-MeNH(CH₂)₃O. |
| 10M | R² is Br and (R^{5b})ₙ is 2,6-di-F, 4-MeNH(CH₂)₃O. |
| 11M | R² is Me and (R^{5b})ₙ is 2,6-di-F, 4-MeNH(CH₂)₃O. |
| 12M | R² is Cl and (R^{5b})ₙ is 2,6-di-F, 4-Me₂N(CH₂)₃O. |
| 13M | R² is Br and (R^{5b})ₙ is 2,6-di-F, 4-Me₂N(CH₂)₃O. |
| 14M | R² is Me and (R^{5b})ₙ is 2,6-di-F, 4-Me₂N(CH₂)₃O. |
| 15M | R² is Cl and (R^{5b})ₙ is 2,6-di-F, 4-MeO(CH₂)₃O. |
| 16M | R² is Br and (R^{5b})ₙ is 2,6-di-F, 4-MeO(CH₂)₃O. |
| 17M | R² is Me and (R^{5b})ₙ is 2,6-di-F, 4-MeO(CH₂)₃O. |

### Formulation/Utility

A compound of this invention will generally be used as a fungicidal active ingredient in a composition, i.e. formulation, with at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2^{nd} Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, triacetin, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2^{nd} Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylenes, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4^{th} Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food—Environment Challenge, T. Brooks and T. R. Roberts, Eds., Proceedings of the 9^{th} International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8^{th} Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Tables A-C. Without further elaboration, it is believed that one skilled in the art using the preceding description can utilize the present invention to its fullest extent. The following Examples are, therefore, to be constructed as merely illustrative, and not limiting of the disclosure in any way whatsoever. Percentages are by weight except where otherwise indicated.

### Example A

### High Strength Concentrate

| | |
|---|---|
| Compound 159 | 98.5% |
| silica aerogel | 0.5% |
| synthetic amorphous fine silica | 1.0% |

### Example B

### Wettable Powder

| | |
|---|---|
| Compound 280 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

### Granule

| | |
|---|---|
| Compound 2 | 10.0% |
| attapulgite granules (low volatile matter, | 90.0% |
| 0.71/0.30 mm; U.S.S. No. 25-50 sieves) | |

### Example D

### Extruded Pellet

| | |
|---|---|
| Compound 37 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

### Emulsifiable Concentrate

| | |
|---|---|
| Compound 122 | 10.0% |
| polyoxyethylene sorbitol hexoleate | 20.0% |
| C₆-C₁₀ fatty acid methyl ester | 70.0% |

### Example F

### Microemulsion

| | |
|---|---|
| Compound 393 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

### Seed Treatment

| | |
|---|---|
| Compound 2 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

Formulations such as those in the Formulation Table are typically diluted with water to form aqueous compositions before application. Aqueous compositions for direct applications to the plant or portion thereof (e.g., spray tank compositions) typically comprise at least about 1 ppm or more (e.g., from 1 ppm to 100 ppm) of the compound(s) of this invention.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, turf, vegetable, field, cereal, and fruit crops. These pathogens include: Oomycetes, including Phytophthora diseases such as Phytophthora infestans, Phytophthora megasperma, Phytophthora parasitica, Phytophthora cinnamomi and Phytophthora capsici, Pythium diseases such as Pythium aphanidermatum, and diseases in the Peronosporaceae family such as Plasmopara viticola, Peronospora spp. (including Peronospora tabacina and Peronospora parasitica), Pseudoperonospora spp. (including Pseudoperonospora cubensis) and Bremia lactucae; Ascomycetes, including Alternaria diseases such as Alternaria solani and Alternaria brassicae, Guignardia diseases such as Guignardia bidwell, Venturia diseases such as Venturia inaequalis, Septoria diseases such as Septoria nodorum and Septoria tritici, powdery mildew diseases such as Erysiphe spp. (including Erysiphe graminis and Erysiphe polygoni), Uncinula necatur, Sphaerotheca fuligena and Podosphaera leucotricha, Pseudocercosporella herpotrichoides, Botrytis diseases such as Botrytis cinerea, Monilinia fructicola, Sclerotinia diseases such as Sclerotinia sclerotiorum, Magnaporthe grisea, Phomopsis viticola, Helminthosporium diseases such as Helminthosporium tritici repentis, Pyrenophora teres, anthracnose diseases such as Glomerella or Colletotrichum spp. (such as Colletotrichum graminicola and Colletotrichum orbiculare), and Gaeumannomyces graminis; Basidiomycetes, including rust diseases caused by Puccinia spp. (such as Puccinia recondite, Puccinia striiformis, Puccinia hordei, Puccinia graminis and Puccinia arachidis), Hemileia vastatrix and Phakopsora pachyrhizi; other pathogens including Rutstroemia floccosum (also known as Sclerontina homoeocarpa); Rhizoctonia spp. (such as Rhizoctonia solani); Fusarium diseases such as Fusarium roseum, Fusarium graminearum and Fusarium oxysporum; Verticillium dahliae; Sclerotium rolfsii; Rynchosporium secalis; Cercosporidium personatum, Cercospora arachidicola and Cercospora beticola; and other genera and species closely related to these pathogens. In addition to their fungicidal activity, the compositions or combinations also have activity against bacteria such as Erwinia amylovora, Xanthomonas campestris, Pseudomonas syringae, and other related species.

Plant disease control is ordinarily accomplished by applying an effective amount of a compound of this invention either pre- or post-infection, to the portion of the plant to be protected such as the roots, stems, foliage, fruit, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants.

Rates of application for these mixtures and compositions of this invention can be influenced by many factors of the environment and should be determined under actual use conditions. Foliage can normally be protected when treated at a rate of from less than about 1 g/ha to about 5,000 g/ha of active ingredients. Seed and seedlings can normally be protected when seed is treated at a rate of from about 0.1 to about 10 g per kilogram of seed; and vegetative propagation units (e.g., cuttings and tubers) can normally be protected when propagation unit is treated at a rate of from about 0.1 to about 10 g per kilogram of propagation unit.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a fungicidally effective amount of a compound of Formula 1 and a biologically effective amount of at least one additional biologically active compound or agent and can further comprise at least one of a surfactant, a solid diluent or a liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

Of note is a composition which in addition to the compound(s) of Formula 1 include at least one fungicidal compound selected from the group consisting of the classes (1) methyl benzimidazole carbamate (MBC) fungicides; (2) dicarboximide fungicides; (3) demethylation inhibitor (DMI) fungicides; (4) phenylamide fungicides; (5) amine/morpholine fungicides; (6) phospholipid biosynthesis inhibitor fungicides; (7) carboxamide fungicides; (8) hydroxy(2-amino-)pyrimidine fungicides; (9) anilinopyrimidine fungicides; (10) N-phenyl carbamate fungicides; (11) quinone outside inhibitor (QoI) fungicides; (12) phenylpyrrole fungicides; (13) quinoline fungicides; (14) lipid peroxidation inhibitor fungicides; (15) melanin biosynthesis inhibitors-reductase (MBI-R) fungicides; (16) melanin biosynthesis inhibitors-dehydratase (MBI-D) fungicides; (17) hydroxyanilide fungicides; (18) squalene-epoxidase inhibitor fungicides; (19) polyoxin fungicides; (20) phenylurea fungicides; (21) quinone inside inhibitor (QiI) fungicides; (22) benzamide fungicides; (23) enopyranuronic acid antibiotic fungicides; (24) hexopyranosyl antibiotic fungicides; (25) glucopyranosyl antibiotic: protein synthesis fungicides; (26) glucopyranosyl antibiotic: trehalase and inositol biosynthesis fungicides; (27) cyanoacetamideoxime fungicides; (28) carbamate fungicides; (29) oxidative phosphorylation uncoupling fungicides; (30) organo tin fungicides; (31) carboxylic acid fungicides; (32) heteroaromatic fungicides; (33) phosphonate fungicides; (34) phthalamic acid fungicides; (35) benzotriazine fungicides; (36) benzene-sulfonamide fungicides; (37) pyridazinone fungicides; (38) thiophene-carboxamide fungicides; (39) pyrimidinamide fungicides; (40) carboxylic acid amide (CAA) fungicides; (41) tetracycline antibiotic fungicides; (42) thiocarbamate fungicides; (43) benzamide fungicides; (44) host plant defense induction fungicides; (45) multi-site contact activity fungicides; (46) fungicides other than classes (1) through (45); and salts of compounds of classes (1) through (46).

Further descriptions of these classes of fungicidal compounds are provided below.

(1) “Methyl benzimidazole carbamate (MBC) fungicides” (Fungicide Resistance Action Committee (FRAC) code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(2) “Dicarboximide fungicides” (Fungicide Resistance Action Committee (FRAC) code 2) are proposed to inhibit a lipid peroxidation in fungi through interference with NADH cytochrome c reductase. Examples include chlozolinate, iprodione, procymidone and vinclozolin.

(3) “Demethylation inhibitor (DMI) fungicides” (Fungicide Resistance Action Committee (FRAC) code 3) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: azoles (including triazoles and imidazoles), pyrimidines, piperazines and pyridines. The triazoles include azaconazole, bitertanol, bromuconazole, cyproconazole, difenoconazole, diniconazole (including diniconazole-M), epoxiconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, triticonazole and uniconazole. The imidazoles include clotrimazole, imazalil, oxpoconazole, prochloraz, pefurazoate and triflumizole. The pyrimidines include fenarimol and nuarimol. The piperazines include triforine. The pyridines include pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(4) “Phenylamide fungicides” (Fungicide Resistance Action Committee (FRAC) code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M, furalaxyl, metalaxyl and metalaxyl-M/mefenoxam. The oxazolidinones include oxadixyl. The butyrolactones include ofurace.

(5) “Amine/morpholine fungicides” (Fungicide Resistance Action Committee (FRAC) code 5) inhibit two target sites within the sterol biosynthetic pathway, Δ⁸→Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(6) “Phospholipid biosynthesis inhibitor fungicides” (Fungicide Resistance Action Committee (FRAC) code 6) inhibit growth of fungi by affecting phospholipid biosynthesis. Phospholipid biosynthesis fungicides include phosphorothiolate and dithiolane fungicides. The phosphorothiolates include edifenphos, iprobenfos and pyrazophos. The dithiolanes include isoprothiolane.

(7) “Carboxamide fungicides” (Fungicide Resistance Action Committee (FRAC) code 7) inhibit Complex II (succinate dehydrogenase) fungal respiration by disrupting a key enzyme in the Krebs Cycle (TCA cycle) named succinate dehydrogenase. Inhibiting respiration prevents the fungus from making ATP, and thus inhibits growth and reproduction. Carboxamide fungicides include benzamides, furan carboxamides, oxathiin carboxamides, thiazole carboxamides, pyrazole carboxamides and pyridine carboxamides. The benzamides include benodanil, flutolanil and mepronil. The furan carboxamides include fenfuram. The oxathiin carboxamides include carboxin and oxycarboxin. The thiazole carboxamides include thifluzamide. The pyrazole carboxamides include furametpyr, penthiopyrad, bixafen, N-[2-(1S,2R)-[1,1′-bicyclopropyl]-2-ylphenyl]-3-(difluoromethyl)-1-methyl-1H-pyrazole-4-carboxamide and N-[2-(1,3-dimethylbutyl)phenyl]-5-fluoro-1,3-dimethyl-1H-pyrazole-4-carboxamide. The pyridine carboxamides include boscalid.

(8) “Hydroxy(2-amino-)pyrimidine fungicides” (Fungicide Resistance Action Committee (FRAC) code 8) inhibit nucleic acid synthesis by interfering with adenosine deaminase. Examples include bupirimate, dimethirimol and ethirimol.

(9) “Anilinopyrimidine fungicides” (Fungicide Resistance Action Committee (FRAC) code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(10) “N-Phenyl carbamate fungicides” (Fungicide Resistance Action Committee (FRAC) code 10) inhibit mitosis by binding to β-tubulin and disrupting microtubule assembly. Inhibition of microtubule assembly can disrupt cell division, transport within the cell and cell structure. Examples include diethofencarb.

(11) “Quinone outside inhibitor (QoI) fungicides” (Fungicide Resistance Action Committee (FRAC) code 11) inhibit Complex III mitochondrial respiration in fungi by affecting ubiquinol oxidase. Oxidation of ubiquinol is blocked at the “quinone outside” (Q_{O}) site of the cytochrome bc₁ complex, which is located in the inner mitochondrial membrane of fungi. Inhibiting mitochondrial respiration prevents normal fungal growth and development. Quinone outside inhibitor fungicides (also known as strobilurin fungicides) include methoxyacrylate, methoxycarbamate, oximinoacetate, oximinoacetamide, oxazolidinedione, dihydrodioxazine, imidazolinone and benzylcarbamate fungicides. The methoxyacrylates include azoxystrobin, enestroburin (SYP-Z071) and picoxystrobin. The methoxycarbamates include pyraclostrobin. The oximinoacetates include kresoxim-methyl and trifloxystrobin. The oximinoacetamides include dimoxystrobin, metominostrobin, orysastrobin, α-[methoxyimino]-N-methyl-2-[[[1-[3-(trifluoromethyl)phenyl]ethoxy]-imino]-methyl]-benzeneacetamide and 2-[[[3-(2,6-dichlorophenyl)-1-methyl-2-propen-1-ylidene]-amino]oxy]methyl]-α-(methoxyimino)-N-methylbenzeneacetamide. The oxazolidinediones include famoxadone. The dihydrodioxazines include fluoxastrobin. The imidazolinones include fenamidone. The benzylcarbamates include pyribencarb.

(12) “Phenylpyrrole fungicides” (Fungicide Resistance Action Committee (FRAC) code 12) inhibit a MAP protein kinase associated with osmotic signal transduction in fungi. Fenpiclonil and fludioxonil are examples of this fungicide class.

(13) “Quinoline fungicides” (Fungicide Resistance Action Committee (FRAC) code 13) are proposed to inhibit signal transduction by affecting G-proteins in early cell signaling. They have been shown to interfere with germination and/or appressorium formation in fungi that cause powder mildew diseases. Quinoxyfen is an example of this class of fungicide.

(14) “Lipid peroxidation inhibitor fungicides” (Fungicide Resistance Action Committee (FRAC) code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic carbon and 1,2,4-thiadiazole fungicides. The aromatic carbon fungicides include biphenyl, chloroneb, dicloran, quintozene, tecnazene and tolclofos-methyl. The 1,2,4-thiadiazole fungicides include etridiazole.

(15) “Melanin biosynthesis inhibitors-reductase (MBI-R) fungicides” (Fungicide Resistance Action Committee (FRAC) code 16.1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

(16) “Melanin biosynthesis inhibitors-dehydratase (MBI-D) fungicides” (Fungicide Resistance Action Committee (FRAC) code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(17) “Hydroxyanilide fungicides (Fungicide Resistance Action Committee (FRAC) code 17) inhibit C4-demethylase which plays a role in sterol production. Examples include fenhexamid.

(18) “Squalene-epoxidase inhibitor fungicides” (Fungicide Resistance Action Committee (FRAC) code 18) inhibit squalene-epoxidase in ergosterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(19) “Polyoxin fungicides” (Fungicide Resistance Action Committee (FRAC) code 19) inhibit chitin synthase. Examples include polyoxin.

(20) “Phenylurea fungicides” (Fungicide Resistance Action Committee (FRAC) code 20) are proposed to affect cell division. Examples include pencycuron.

(21) “Quinone inside inhibitor (QiI) fungicides” (Fungicide Resistance Action Committee (FRAC) code 21) inhibit Complex III mitochondrial respiration in fungi by affecting ubiquinol reductase. Reduction of ubiquinol is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(22) “Benzamide fungicides” (Fungicide Resistance Action Committee (FRAC) code 22) inhibit mitosis by binding to β-tubulin and disrupting microtubule assembly. Inhibition of microtubule assembly can disrupt cell division, transport within the cell and cell structure. Examples include zoxamide.

(23) “Enopyranuronic acid antibiotic fungicides” (Fungicide Resistance Action Committee (FRAC) code 23) inhibit growth of fungi by affecting protein biosynthesis. Examples include blasticidin-S.

(24) “Hexopyranosyl antibiotic fungicides” (Fungicide Resistance Action Committee (FRAC) code 24) inhibit growth of fungi by affecting protein biosynthesis. Examples include kasugamycin.

(25) “Glucopyranosyl antibiotic: protein synthesis fungicides” (Fungicide Resistance Action Committee (FRAC) code 25) inhibit growth of fungi by affecting protein biosynthesis. Examples include streptomycin.

(26) “Glucopyranosyl antibiotic: trehalase and inositol biosynthesis fungicides” (Fungicide Resistance Action Committee (FRAC) code 26) inhibit trehalase in inositol biosynthesis pathway. Examples include validamycin.

(27) “Cyanoacetamideoxime fungicides (Fungicide Resistance Action Committee (FRAC) code 27) include cymoxanil.

(28) “Carbamate fungicides” (Fungicide Resistance Action Committee (FRAC) code 28) are considered multi-site inhibitors of fungal growth. They are proposed to interfere with the synthesis of fatty acids in cell membranes, which then disrupts cell membrane permeability. Propamacarb, propamacarb-hydrochloride, iodocarb, and prothiocarb are examples of this fungicide class.

(29) “Oxidative phosphorylation uncoupling fungicides” (Fungicide Resistance Action Committee (FRAC) code 29) inhibit fungal respiration by uncoupling oxidative phosphorylation. Inhibiting respiration prevents normal fungal growth and development. This class includes 2,6-dinitroanilines such as fluazinam, pyrimidonehydrazones such as ferimzone and dinitrophenyl crotonates such as dinocap, meptyldinocap and binapacryl.

(30) “Organo tin fungicides” (Fungicide Resistance Action Committee (FRAC) code 30) inhibit adenosine triphosphate (ATP) synthase in oxidative phosphorylation pathway. Examples include fentin acetate, fentin chloride and fentin hydroxide.

(31) “Carboxylic acid fungicides” (Fungicide Resistance Action Committee (FRAC) code 31) inhibit growth of fungi by affecting deoxyribonucleic acid (DNA) topoisomerase type II (gyrase). Examples include oxolinic acid.

(32) “Heteroaromatic fungicides” (Fungicide Resistance Action Committee (FRAC) code 32) are proposed to affect DNA/ribonucleic acid (RNA) synthesis. Heteroaromatic fungicides include isoxazole and isothiazolone fungicides. The isoxazoles include hymexazole and the isothiazolones include octhilinone.

(33) “Phosphonate fungicides” (Fungicide Resistance Action Committee (FRAC) code 33) include phosphorous acid and its various salts, including fosetyl-aluminum.

(34) “Phthalamic acid fungicides” (Fungicide Resistance Action Committee (FRAC) code 34) include teclofthalam.

(35) “Benzotriazine fungicides” (Fungicide Resistance Action Committee (FRAC) code 35) include triazoxide.

(36) “Benzene-sulfonamide fungicides” (Fungicide Resistance Action Committee (FRAC) code 36) include flusulfamide.

(37) “Pyridazinone fungicides” (Fungicide Resistance Action Committee (FRAC) code 37) include diclomezine.

(38) “Thiophene-carboxamide fungicides” (Fungicide Resistance Action Committee (FRAC) code 38) are proposed to affect ATP production. Examples include silthiofam.

(39) “Pyrimidinamide fungicides” (Fungicide Resistance Action Committee (FRAC) code 39) inhibit growth of fungi by affecting phospholipid biosynthesis and include diflumetorim.

(40) “Carboxylic acid amide (CAA) fungicides” (Fungicide Resistance Action Committee (FRAC) code 40) are proposed to inhibit phospholipid biosynthesis and cell wall deposition. Inhibition of these processes prevents growth and leads to death of the target fungus. Carboxylic acid amide fungicides include cinnamic acid amide, valinamide carbamate and mandelic acid amide fungicides. The cinnamic acid amides include dimethomorph and flumorph. The valinamide carbamates include benthiavalicarb, benthiavalicarb-isopropyl, iprovalicarb and valiphenal. The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)amino]butanamide and N-[2-[4-[[3-(4-chlorophenyl)-2-propyn-1-yl]oxy]-3-methoxyphenyl]ethyl]-3-methyl-2-[(ethylsulfonyl)amino]butanamide.

(41) “Tetracycline antibiotic fungicides” (Fungicide Resistance Action Committee (FRAC) code 41) inhibit growth of fungi by affecting complex 1 nicotinamide adenine dinucleotide (NADH) oxidoreductase. Examples include oxytetracycline.

(42) “Thiocarbamate fungicides (b42)” (Fungicide Resistance Action Committee (FRAC) code 42) include methasulfocarb.

(43) “Benzamide fungicides” (Fungicide Resistance Action Committee (FRAC) code 43) inhibit growth of fungi by delocalization of spectrin-like proteins. Examples include acylpicolide fungicides such as fluopicolide and fluopyram.

(44) “Host plant defense induction fungicides” (Fungicide Resistance Action Committee (FRAC) code P) induce host plant defense mechanisms. Host plant defense induction fungicides include benzo-thiadiazole, benzisothiazole and thiadiazole-carboxamide fungicides. The benzo-thiadiazoles include acibenzolar-5-methyl. The benzisothiazoles include probenazole. The thiadiazole-carboxamides include tiadinil and isotianil.

(45) “Multi-site contact fungicides” inhibit fungal growth through multiple sites of action and have contact/preventive activity. This class of fungicides includes: (45.1) “copper fungicides” (Fungicide Resistance Action Committee (FRAC) code M1)”, (45.2) “sulfur fungicides” (Fungicide Resistance Action Committee (FRAC) code M2), (45.3) “dithiocarbamate fungicides” (Fungicide Resistance Action Committee (FRAC) code M3), (45.4) “phthalimide fungicides” (Fungicide Resistance Action Committee (FRAC) code M4), (45.5) “chloronitrile fungicides” (Fungicide Resistance Action Committee (FRAC) code M5), (45.6) “sulfamide fungicides” (Fungicide Resistance Action Committee (FRAC) code M6), (45.7) “guanidine fungicides” (Fungicide Resistance Action Committee (FRAC) code M7), (45.8) “triazine fungicides” (Fungicide Resistance Action Committee (FRAC) code M8) and (45.9) “quinone fungicides” (Fungicide Resistance Action Committee (FRAC) code M9). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. “Guanidine fungicides” include dodine, guazatine, iminoctadine albesilate and iminoctadine triacetate. “Triazine fungicides” include anilazine. “Quinone fungicides” include dithianon.

(46) “Fungicides other than fungicides of classes (1) through (45)” include certain fungicides whose mode of action may be unknown. These include: (46.1) “thiazole carboxamide fungicides” (Fungicide Resistance Action Committee (FRAC) code U5), (46.2) “phenyl-acetamide fungicides” (Fungicide Resistance Action Committee (FRAC) code U6), (46.3) “quinazolinone fungicides” (Fungicide Resistance Action Committee (FRAC) code U7) and (46.4) “benzophenone fungicides” (Fungicide Resistance Action Committee (FRAC) code U8). The thiazole carboxamides include ethaboxam. The phenyl-acetamides include cyflufenamid and N-[[cyclopropylmethoxy)amino][6-(difluoromethoxy)-2,3-difluorophenyl]-methylene]benzeneacetamide. The quinazolinones include proquinazid and 2-butoxy-6-iodo-3-propyl-4H-1-benzopyran-4-one. The benzophenones include metrafenone. The (b46) class also includes bethoxazin, neo-asozin (ferric methanearsonate), pyrroInitrin, quinomethionate, N-[2-[4-[[3-(4-chlorophenyl)-2-propyn-1-yl]oxy]-3-methoxy-phenyl]ethyl]-3-methyl-2-[(methylsulfonyl)amino]butanamide, N-[2-[4-[[3-(4-chloro-phenyl)-2-propyn-1-yl]oxy]-3-methoxyphenyl]ethyl]-3-methyl-2-[(ethyl-sulfonyl)amino]-butanamide, 2-[[2-fluoro-5-(trifluoromethyl)phenyl]thio]-2-[3-(2-methoxyphenyl)-2-thiazo-lidinylidene]acetonitrile, 3-[5-(4-chlorophenyl)-2,3-dimethyl-3-isoxazolidinyl]pyridine, 4-fluoro-phenyl N-[1-[[[1-(4-cyanophenyl)ethyl]sulfonyl]methyl]propyl]carbamate, 5-chloro-6-(2,4,6-trifluorophenyl)-7-(4-methylpiperidin-1-yl)[1,2,4]triazolo[1,5-a]pyrimidine, N-(4-chloro-2-nitrophenyl)-N-ethyl-4-methylbenzenesulfonamide, N-[[(cyclopropyl-methoxy)amino][6-(difluoromethoxy)-2,3-difluorophenyl]methylene]benzeneacetamide, N′-[4-[4-chloro-3-(trifluoro-methyl)phenoxy]-2,5-dimethylphenyl]-N-ethyl-N-methyl-methanimid-amide, 1-[(2-propenylthio)carbonyl]-2-(1-methylethyl)-4-(2-methylphenyl)-5-amino-1H-pyrazol-3-one, 3-(difluoromethyl)-1-methyl-N-(3′,4′,5′-trifluoro[1,1′-biphenyl]-2-yl)-1H-pyrazole-4-carboxamide, 5-ethyl-6-octyl-[1,2,4]triazole[1,5-a]pyrimidin-7-amine and Initium®.

Therefore of note is a mixture (i.e. composition) comprising a compound(s) of Formula 1 and at least one fungicidal compound selected from the group consisting of the aforedescribed classes (1) through (46). Examples include compositions comprising at least one fungicidal compound selected from aforedescribed class (18), compositions comprising at least one fungicidal compound selected from aforedescribed class (19), compositions comprising at least one fungicidal compound selected from aforedescribed class (21), compositions comprising at least one fungicidal compound selected from aforedescribed class (25), compositions comprising at least one fungicidal compound selected from aforedescribed class (31), compositions comprising at least one fungicidal compound selected from aforedescribed class (34), compositions comprising at least one fungicidal compound selected from aforedescribed class (38), compositions comprising at least one fungicidal compound selected from aforedescribed class (39), compositions comprising at least one fungicidal compound selected from aforedescribed class (41), compositions comprising at least one fungicidal compound selected from aforedescribed class (45.7) and compositions comprising at least one fungicidal compound selected from aforedescribed class (45.9); including but not limited to such compositions comprising a compound(s) of Formula 1 wherein J is Q², X is CR², Y is N and Z is CR⁴, Q² is an optionally substituted phenyl ring, an optionally substituted naphthalenyl ring system, an optionally substituted fully unsaturated heterocyclic ring, or an optionally substituted heteroaromatic bicyclic ring system, R² is halogen and Q¹ is a phenyl ring or a 2-pyridinyl ring substituted with halogen at an ortho position; and such compositions comprising a compound(s) of Formula 1 wherein J is Q², X is CR², Y is N and Z is CR⁴, Q¹ is an optionally substituted phenyl ring, an optionally substituted naphthalenyl ring system, an optionally substituted fully unsaturated heterocyclic ring, or an optionally substituted heteroaromatic bicyclic ring system, R⁴ is halogen and Q² is a phenyl ring or a 2-pyridinyl ring substituted with halogen at an ortho position.

Also of note is a composition comprising said mixture (in fungicidally effective amount) and further comprising at least one additional component selected from the group consisting of surfactants, solid diluents and liquid diluents. Of particular note is a mixture (i.e. composition) comprising a compound(s) of Formula 1 and at least one fungicidal compound selected from the group of specific compounds listed above in connection with classes (1) through (46). Also of particular note is a composition comprising said mixture (in fungicidally effective amount) and further comprising at least one additional surfactant selected from the group consisting of surfactants, solid diluents and liquid diluents.

Of particular note are compositions which in addition to compound(s) of Formula 1 include at least one compound selected from the group consisting of (1) alkylenebis(dithiocarbamate) fungicides; (2) cymoxanil; (3) phenylamide fungicides; (4) pyrimidinone fungicides; (5) chlorothalonil; (6) carboxamides acting at complex II of the fungal mitochondrial respiratory electron transfer site; (7) quinoxyfen; (8) metrafenone; (9) cyflufenamid; (10) cyprodinil; (11) copper compounds; (12) phthalimide fungicides; (13) fosetyl-aluminum; (14) benzimidazole fungicides; (15) cyazofamid; (16) fluazinam; (17) iprovalicarb; (18) propamocarb; (19) validomycin; (20) dichlorophenyl dicarboximide fungicides; (21) zoxamide; (22) fluopicolide; (23) mandipropamid; (24) carboxylic acid amides acting on phospholipid biosynthesis and cell wall deposition; (25) dimethomorph; (26) non-DMI sterol biosynthesis inhibitors; (27) inhibitors of demethylase in sterol biosynthesis; (28) bc₁ complex fungicides; and salts of compounds of (1) through (28).

Further descriptions of classes of fungicidal compounds are provided below.

Pyrimidinone fungicides (group (4)) include compounds of Formula A1

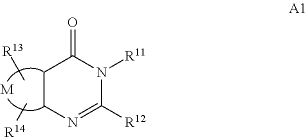

wherein M forms a fused phenyl, thiophene or pyridine ring; R¹¹ is C₁-C₆ alkyl; R¹² is C₁-C₆ alkyl or C₁-C₆ alkoxy; R¹³ is halogen; and R¹⁴ is hydrogen or halogen.

Pyrimidinone fungicides are described in PCT Patent Application Publication WO 94/26722 and U.S. Pat. Nos. 6,066,638, 6,245,770, 6,262,058 and 6,277,858. Of note are pyrimidinone fungicides selected from the group: 6-bromo-3-propyl-2-propyloxy-4(3H)-quinazolinone, 6,8-diiodo-3-propyl-2-propyloxy-4(3H)-quinazolinone, 6-iodo-3-propyl-2-propyloxy-4(3H)-quinazolinone (proquinazid), 6-chloro-2-propoxy-3-propyl-thieno-[2,3-d]pyrimidin-4(3H)-one, 6-bromo-2-propoxy-3-propylthieno[2,3-d]pyrimidin-4(3H)-one, 7-bromo-2-propoxy-3-propylthieno[3,2-d]pyrimidin-4(3H)-one, 6-bromo-2-propoxy-3-propylpyrido[2,3-d]pyrimidin-4(3H)-one, 6,7-dibromo-2-propoxy-3-propyl-thieno-[3,2-d]pyrimidin-4(3H)-one, and 3-(cyclopropylmethyl)-6-iodo-2-(propyl-thio)-pyrido-[2,3-d]pyrimidin-4(3H)-one.

Sterol biosynthesis inhibitors (group (27)) control fungi by inhibiting enzymes in the sterol biosynthesis pathway. Demethylase-inhibiting fungicides have a common site of action within the fungal sterol biosynthesis pathway, involving inhibition of demethylation at position 14 of lanosterol or 24-methylene dihydrolanosterol, which are precursors to sterols in fungi. Compounds acting at this site are often referred to as demethylase inhibitors, DMI fungicides, or DMIs. The demethylase enzyme is sometimes referred to by other names in the biochemical literature, including cytochrome P-450 (14DM). The demethylase enzyme is described in, for example, J. Biol. Chem. 1992, 267, 13175-79 and references cited therein. DMI fungicides are divided between several chemical classes: azoles (including triazoles and imidazoles), pyrimidines, piperazines and pyridines. The triazoles include azaconazole, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole and uniconazole. The imidazoles include clotrimazole, econazole, imazalil, isoconazole, miconazole, oxpoconazole, prochloraz and triflumizole. The pyrimidines include fenarimol, nuarimol and triarimol. The piperazines include triforine. The pyridines include buthiobate and pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

bc₁ Complex Fungicides (group (28)) have a fungicidal mode of action which inhibits the bc₁ complex in the mitochondrial respiration chain. The bc₁ complex is sometimes referred to by other names in the biochemical literature, including complex III of the electron transfer chain, and ubihydroquinone:cytochrome c oxidoreductase. This complex is uniquely identified by Enzyme Commission number EC1.10.2.2. The bc₁ complex is described in, for example, J. Biol. Chem. 1989, 264, 14543-48; Methods Enzymol. 1986, 126, 253-71; and references cited therein. Strobilurin fungicides such as azoxystrobin, dimoxystrobin, enestroburin (SYP-Z071), fluoxastrobin, kresoxim-methyl, metominostrobin, orysastrobin, picoxystrobin, pyraclostrobin and trifloxystrobin are known to have this mode of action (H. Sauter et al., Angew. Chem. Int. Ed. 1999, 38, 1328-1349). Other fungicidal compounds that inhibit the bc₁ complex in the mitochondrial respiration chain include famoxadone and fenamidone.

Alkylenebis(dithiocarbamate)s (group (1)) include compounds such as mancozeb, maneb, propineb and zineb. Phenylamides (group (3)) include compounds such as metalaxyl, benalaxyl, furalaxyl and oxadixyl. Carboxamides (group (6)) include compounds such as boscalid, carboxin, fenfuram, flutolanil, furametpyr, mepronil, oxycarboxin, thifluzamide, penthiopyrad and N-[2-(1,3-dimethylbutyl)phenyl]-5-fluoro-1,3-dimethyl-1H-pyrazole-4-carboxamide (PCT Patent Publication WO 2003/010149), and are known to inhibit mitochondrial function by disrupting complex II (succinate dehydrogenase) in the respiratory electron transport chain. Copper compounds (group (11)) include compounds such as copper oxychloride, copper sulfate and copper hydroxide, including compositions such as Bordeaux mixture (tribasic copper sulfate). Phthalimides (group (12)) include compounds such as folpet and captan. Benzimidazole fungicides (group (14)) include benomyl and carbendazim. Dichlorophenyl dicarboximide fungicides (group (20)) include chlozolinate, dichlozoline, iprodione, isovaledione, myclozolin, procymidone and vinclozolin.

Non-DMI sterol biosynthesis inhibitors (group (26)) include morpholine and piperidine fungicides. The morpholines and piperidines are sterol biosynthesis inhibitors that have been shown to inhibit steps in the sterol biosynthesis pathway at a point later than the inhibitions achieved by the DMI sterol biosynthesis (group (27)). The morpholines include aldimorph, dodemorph, fenpropimorph, tridemorph and trimorphamide. The piperidines include fenpropidin

Of further note are combinations of compound(s) of Formula 1 with azoxystrobin, kresoxim-methyl, trifloxystrobin, pyraclostrobin, picoxystrobin, dimoxystrobin, metominostrobinifenominostrobin, carbendazim, chlorothalonil, quinoxyfen, metrafenone, cyflufenamid, fenpropidine, fenpropimorph, bromuconazole, cyproconazole, difenoconazole, epoxiconazole, fenbuconazole, flusilazole, hexaconazole, ipconazole, metconazole, penconazole, propiconazole, proquinazid, prothioconazole, tebuconazole, triticonazole, famoxadone, prochloraz, penthiopyrad and boscalid (nicobifen).

Preferred for better control of plant diseases caused by fungal plant pathogens (e.g., lower use rate or broader spectrum of plant pathogens controlled) or resistance management are mixtures of a compound of this invention with a fungicide selected from the group: azoxystrobin, kresoxim-methyl, trifloxystrobin, pyraclostrobin, picoxystrobin, dimoxystrobin, metominostrobinifenominostrobin, quinoxyfen, metrafenone, cyflufenamid, fenpropidine, fenpropimorph, cyproconazole, epoxiconazole, flusilazole, metconazole, propiconazole, proquinazid, prothioconazole, tebuconazole, triticonazole, famoxadone and penthiopyrad.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective control is always desirable. When synergism of fungicidal active ingredients occurs at application rates giving agronomically satisfactory levels of fungal control, such combinations can be advantageous for reducing crop production cost and decreasing environmental load.

Of note is a combination of a compound(s) of Formula 1 with at least one other fungicidal active ingredient. Of particular note is such a combination where the other fungicidal active ingredient has different site of action from the compound(s) of Formula 1. In certain instances, a combination with at least one other fungicidal active ingredient having a similar spectrum of control but a different site of action will be particularly advantageous for resistance management. Thus, a composition of the present invention can further comprise a biologically effective amount of at least one additional fungicidal active ingredient having a similar spectrum of control but a different site of action.

When one or more of these various mixing partners are used, the weight ratio of these various mixing partners (in total) to the compound(s) of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

Specific weight ratios illustrative of the mixtures, compositions and methods of the present invention are listed in Table A1. The first column of Table A1 lists the specific mixing partner compound (e.g., “Acibenzolar-5-methyl” in the first line). The second, third and fourth columns of Table A1 lists ranges of weight ratios for rates at which the mixing partner compound is typically applied relative to a compound(s) of Formula 1. Thus, for example, the first line of Table A1 specifically discloses the combination of a compound(s) of Formula 1 with acibenzolar-5-methyl is typically applied in a weight ratio between 1:4 to 210:1. The remaining lines of Table A1 are to be construed similarly.

TABLE A1

| | Typical | More Typical | Most Typical |
|---|---|---|---|
| Mixing Partner Compound | Weight Ratio | Weight Ratio | Weight Ratio |
| acibenzolar-S-methyl | 1:4 to 210:1 | 1:1 to 70:1 | 1:1 to 11:1 |
| aldimorph | 1:60 to 4:1 | 1:20 to 2:1 | 1:12 to 1:2 |
| amisulbrom | 1:12 to 21:1 | 1:4 to 7:1 | 1:2 to 4:1 |
| anilazine | 1:180 to 2:1 | 1:60 to 1:3 | 1:36 to 1:6 |
| azaconazole | 1:15 to 21:1 | 1:5 to 7:1 | 1:3 to 3:1 |
| azoxystrobin | 1:18 to 14:1 | 1:6 to 5:1 | 1:4 to 2:1 |
| benalaxyl | 1:9 to 21:1 | 1:3 to 7:1 | 1:2 to 4:1 |
| benalaxyl-M | 1:9 to 42:1 | 1:3 to 14:1 | 1:1 to 5:1 |
| benodanil | 1:36 to 7:1 | 1:12 to 3:1 | 1:7 to 2:1 |
| benomyl | 1:90 to 5:1 | 1:30 to 2:1 | 1:18 to 1:2 |
| benthiavalicarb | 1:4 to 42:1 | 1:1 to 14:1 | 1:1 to 8:1 |
| benthiavalicarb-isopropyl | 1:4 to 42:1 | 1:1 to 14:1 | 1:1 to 8:1 |
| bethoxazin | 1:300 to 42:1 | 1:100 to 14:1 | 1:24 to 1:3 |
| binapacryl | 1:300 to 42:1 | 1:100 to 14:1 | 1:24 to 1:3 |
| biphenyl | 1:300 to 42:1 | 1:100 to 14:1 | 1:24 to 1:3 |
| bitertanol | 1:30 to 6:1 | 1:10 to 2:1 | 1:6 to 1:2 |
| bixafen | 1:24 to 11:1 | 1:8 to 4:1 | 1:3 to 2:1 |
| blasticidin-S | 1:6 to 105:1 | 1:2 to 35:1 | 2:1 to 19:1 |
| Bordeaux mixture (tribasic copper sulfate) | 1:900 to 2:1 | 1:300 to 1:3 | 1:72 to 1:8 |
| boscalid | 1:36 to 7:1 | 1:12 to 3:1 | 1:7 to 2:1 |
| bromuconazole | 1:30 to 11:1 | 1:10 to 4:1 | 1:4 to 2:1 |
| bupirimate | 1:6 to 105:1 | 1:2 to 35:1 | 2:1 to 19:1 |
| captafol | 1:180 to 5:1 | 1:60 to 2:1 | 1:24 to 1:3 |
| captan | 1:180 to 5:1 | 1:60 to 2:1 | 1:24 to 1:3 |
| carbendazim | 1:90 to 5:1 | 1:30 to 2:1 | 1:18 to 1:2 |
| carboxin | 1:36 to 7:1 | 1:12 to 3:1 | 1:7 to 2:1 |
| carpropamid | 1:30 to 11:1 | 1:10 to 4:1 | 1:4 to 2:1 |
| chloroneb | 1:600 to 2:1 | 1:200 to 1:3 | 1:160 to 1:22 |
| chlorothalonil | 1:180 to 5:1 | 1:60 to 2:1 | 1:24 to 1:3 |
| chlozolinate | 1:90 to 3:1 | 1:30 to 1:2 | 1:18 to 1:3 |
| clotrimazole | 1:30 to 11:1 | 1:10 to 4:1 | 1:4 to 2:1 |
| copper oxychloride | 1:450 to 2:1 | 1:150 to 1:3 | 1:96 to 1:11 |
| copper salts such as copper sulfate and copper | 1:240 to 6:1 | 1:80 to 2:1 | 1:12 to 1:2 |
| hydroxide | | | |
| cyazofamid | 1:9 to 21:1 | 1:3 to 7:1 | 1:1 to 4:1 |
| cyflufenamid | 1:3 to 105:1 | 1:1 to 35:1 | 1:1 to 15:1 |
| cymoxanil | 1:12 to 21:1 | 1:4 to 7:1 | 1:2 to 4:1 |
| cyproconazole | 1:9 to 21:1 | 1:3 to 7:1 | 1:1 to 4:1 |
| cyprodinil | 1:45 to 11:1 | 1:15 to 4:1 | 1:7 to 2:1 |
| dichlofluanid | 1:300 to 42:1 | 1:100 to 14:1 | 1:24 to 1:3 |
| diclocymet | 1:300 to 42:1 | 1:100 to 14:1 | 1:24 to 1:3 |
| diclomezine | 1:30 to 11:1 | 1:10 to 4:1 | 1:6 to 2:1 |
| dicloran | 1:300 to 42:1 | 1:100 to 14:1 | 1:24 to 1:3 |
| diethofencarb | 1:45 to 11:1 | 1:15 to 4:1 | 1:12 to 1:2 |
| difenoconazole | 1:9 to 42:1 | 1:3 to 14:1 | 1:1 to 8:1 |
| diflumetorim | 1:300 to 42:1 | 1:100 to 14:1 | 1:24 to 1:3 |
| dimethirimol | 1:6 to 105:1 | 1:2 to 35:1 | 2:1 to 19:1 |
| dimethomorph | 1:18 to 7:1 | 1:6 to 3:1 | 1:4 to 2:1 |
| dimoxystrobin | 1:18 to 21:1 | 1:6 to 7:1 | 1:3 to 3:1 |
| diniconazole | 1:6 to 42:1 | 1:2 to 14:1 | 1:1 to 5:1 |
| diniconazole M | 1:6 to 105:1 | 1:2 to 35:1 | 1:1 to 8:1 |
| dinocap | 1:15 to 11:1 | 1:5 to 4:1 | 1:3 to 2:1 |
| dithianon | 1:30 to 5:1 | 1:10 to 2:1 | 1:8 to 1:2 |
| dodemorph | 1:60 to 4:1 | 1:20 to 2:1 | 1:12 to 1:2 |
| dodine | 1:60 to 3:1 | 1:20 to 1:2 | 1:16 to 1:3 |
| edifenphos | 1:60 to 11:1 | 1:20 to 4:1 | 1:6 to 2:1 |
| enestroburin | 1:18 to 21:1 | 1:6 to 7:1 | 1:3 to 3:1 |
| epoxiconazole | 1:7 to 42:1 | 1:2 to 14:1 | 1:2 to 5:1 |
| ethaboxam | 1:15 to 11:1 | 1:5 to 4:1 | 1:3 to 2:1 |
| etridiazole | 1:6 to 105:1 | 1:2 to 35:1 | 2:1 to 19:1 |
| famoxadone | 1:18 to 21:1 | 1:6 to 7:1 | 1:3 to 3:1 |
| fenamidone | 1:12 to 21:1 | 1:4 to 7:1 | 1:3 to 3:1 |
| fenarimol | 1:6 to 105:1 | 1:2 to 35:1 | 1:1 to 15:1 |
| fenbuconazole | 1:6 to 35:1 | 1:2 to 12:1 | 1:1 to 7:1 |
| fenfuram | 1:36 to 7:1 | 1:12 to 3:1 | 1:7 to 2:1 |
| fenhexamid | 1:60 to 3:1 | 1:20 to 1:2 | 1:16 to 1:3 |
| fenoxanil | 1:300 to 42:1 | 1:100 to 14:1 | 1:24 to 1:3 |
| fenpiclonil | 1:150 to 11:1 | 1:50 to 4:1 | 1:24 to 1:3 |
| fenpropidin | 1:60 to 4:1 | 1:20 to 2:1 | 1:12 to 1:2 |
| fenpropimorph | 1:60 to 4:1 | 1:20 to 2:1 | 1:12 to 1:2 |
| fentin acetate | 1:30 to 11:1 | 1:10 to 4:1 | 1:4 to 2:1 |
| fentin chloride | 1:30 to 11:1 | 1:10 to 4:1 | 1:4 to 2:1 |
| fentin hydroxide | 1:30 to 11:1 | 1:10 to 4:1 | 1:4 to 2:1 |
| ferbam | 1:600 to 3:1 | 1:200 to 1:2 | 1:48 to 1:6 |
| ferimzone | 1:60 to 6:1 | 1:20 to 2:1 | 1:12 to 1:2 |
| fluazinam | 1:45 to 6:1 | 1:15 to 2:1 | 1:6 to 1:2 |
| fludioxonil | 1:15 to 14:1 | 1:5 to 5:1 | 1:3 to 3:1 |
| flumetover | 1:18 to 7:1 | 1:6 to 3:1 | 1:4 to 2:1 |
| flumorph | 1:18 to 21:1 | 1:6 to 7:1 | 1:4 to 2:1 |
| fluopicolide | 1:7 to 21:1 | 1:2 to 7:1 | 1:1 to 4:1 |
| fluopyram | 1:30 to 105:1 | 1:10 to 35:1 | 1:4 to 2:1 |
| fluoromide | 1:300 to 2:1 | 1:100 to 1:3 | 1:60 to 1:7 |
| fluoxastrobin | 1:9 to 21:1 | 1:3 to 7:1 | 1:2 to 4:1 |
| fluquinconazole | 1:9 to 14:1 | 1:3 to 5:1 | 1:2 to 3:1 |
| flusilazole | 1:30 to 11:1 | 1:10 to 4:1 | 1:4 to 2:1 |
| flusulfamide | 1:180 to 3:1 | 1:60 to 1:2 | 1:24 to 1:3 |
| flutianil | 1:15 to 42:1 | 1:5 to 14:1 | 1:2 to 4:1 |
| flutolanil | 1:36 to 7:1 | 1:12 to 3:1 | 1:7 to 2:1 |
| flutriafol | 1:9 to 14:1 | 1:3 to 5:1 | 1:2 to 3:1 |
| folpet | 1:180 to 5:1 | 1:60 to 2:1 | 1:24 to 1:3 |
| fosetyl-aluminum | 1:450 to 1:2 | 1:150 to 1:5 | 1:48 to 1:8 |
| fuberidazole | 1:90 to 5:1 | 1:30 to 2:1 | 1:18 to 1:2 |
| furalaxyl | 1:30 to 53:1 | 1:10 to 18:1 | 1:2 to 4:1 |
| furametpyr | 1:300 to 42:1 | 1:100 to 14:1 | 1:24 to 1:3 |
| guazatine | 1:300 to 42:1 | 1:100 to 14:1 | 1:24 to 1:3 |
| hexaconazole | 1:30 to 42:1 | 1:10 to 14:1 | 1:3 to 3:1 |
| hymexazol | 1:450 to 2:1 | 1:150 to 1:3 | 1:120 to 1:14 |
| imazalil | 1:15 to 21:1 | 1:5 to 7:1 | 1:3 to 3:1 |
| imibenconazole | 1:30 to 42:1 | 1:10 to 14:1 | 1:3 to 3:1 |
| iodocarb | 1:150 to 5:1 | 1:50 to 2:1 | 1:24 to 1:3 |
| ipconazole | 1:150 to 5:1 | 1:50 to 2:1 | 1:24 to 1:3 |
| iprobenfos | 1:300 to 42:1 | 1:100 to 14:1 | 1:24 to 1:3 |
| iprodione | 1:240 to 3:1 | 1:80 to 1:2 | 1:24 to 1:3 |
| iprovalicarb | 1:18 to 11:1 | 1:6 to 4:1 | 1:3 to 2:1 |
| isoprothiolane | 1:300 to 2:1 | 1:100 to 1:3 | 1:72 to 1:8 |
| isopyrazam | 1:24 to 11:1 | 1:8 to 4:1 | 1:3 to 2:1 |
| isotianil | 1:24 to 11:1 | 1:8 to 4:1 | 1:3 to 2:1 |
| kasugamycin | 1:15 to 105:1 | 1:5 to 35:1 | 1:1 to 15:1 |
| kresoxim-methyl | 1:15 to 21:1 | 1:5 to 7:1 | 1:3 to 3:1 |
| mancozeb | 1:360 to 3:1 | 1:120 to 1:2 | 1:36 to 1:4 |
| mandipropamid | 1:12 to 21:1 | 1:4 to 7:1 | 1:3 to 3:1 |
| maneb | 1:360 to 3:1 | 1:120 to 1:2 | 1:36 to 1:4 |
| mepanipyrim | 1:36 to 4:1 | 1:12 to 2:1 | 1:9 to 1:2 |
| mepronil | 1:15 to 42:1 | 1:5 to 14:1 | 1:2 to 4:1 |
| meptyldinocap | 1:15 to 11:1 | 1:5 to 4:1 | 1:3 to 2:1 |
| metalaxyl | 1:30 to 53:1 | 1:10 to 18:1 | 1:2 to 4:1 |
| metalaxyl-M | 1:30 to 53:1 | 1:10 to 18:1 | 1:2 to 4:1 |
| metconazole | 1:6 to 21:1 | 1:2 to 7:1 | 1:1 to 4:1 |
| methasulfocarb | 1:300 to 42:1 | 1:100 to 14:1 | 1:24 to 1:3 |
| metiram | 1:300 to 42:1 | 1:100 to 14:1 | 1:24 to 1:3 |
| metominostrobin | 1:18 to 14:1 | 1:6 to 5:1 | 1:4 to 2:1 |
| metrafenone | 1:12 to 14:1 | 1:4 to 5:1 | 1:3 to 3:1 |
| myclobutanil | 1:10 to 30:1 | 1:3 to 10:1 | 1:1 to 5:1 |
| naftifine | 1:300 to 42:1 | 1:100 to 14:1 | 1:24 to 1:3 |
| neo-asozin (ferric methanearsonate) | 1:300 to 42:1 | 1:100 to 14:1 | 1:24 to 1:3 |
| nuarimol | 1:30 to 11:1 | 1:10 to 4:1 | 1:4 to 2:1 |
| octhilinone | 1:300 to 42:1 | 1:100 to 14:1 | 1:24 to 1:3 |
| ofurace | 1:30 to 53:1 | 1:10 to 18:1 | 1:2 to 4:1 |
| orysastrobin | 1:18 to 14:1 | 1:6 to 5:1 | 1:4 to 2:1 |
| oxadixyl | 1:30 to 53:1 | 1:10 to 18:1 | 1:2 to 4:1 |
| oxolinic acid | 1:60 to 11:1 | 1:20 to 4:1 | 1:12 to 1:2 |
| oxpoconazole | 1:30 to 42:1 | 1:10 to 14:1 | 1:3 to 3:1 |
| oxycarboxin | 1:36 to 7:1 | 1:12 to 3:1 | 1:7 to 2:1 |
| oxytetracycline | 1:30 to 11:1 | 1:10 to 4:1 | 1:6 to 2:1 |
| pefurazoate | 1:300 to 42:1 | 1:100 to 14:1 | 1:24 to 1:3 |
| penconazole | 1:3 to 53:1 | 1:1 to 18:1 | 1:1 to 10:1 |
| pencycuron | 1:300 to 3:1 | 1:100 to 1:2 | 1:18 to 1:3 |
| penthiopyrad | 1:24 to 11:1 | 1:8 to 4:1 | 1:3 to 2:1 |
| phosphorous acid and salts | 1:300 to 42:1 | 1:100 to 14:1 | 1:24 to 1:3 |
| phthalide | 1:300 to 42:1 | 1:100 to 14:1 | 1:24 to 1:3 |
| picoxystrobin | 1:15 to 21:1 | 1:5 to 7:1 | 1:3 to 3:1 |
| piperalin | 1:30 to 11:1 | 1:10 to 4:1 | 1:4 to 2:1 |
| polyoxin | 1:30 to 11:1 | 1:10 to 4:1 | 1:6 to 2:1 |
| probenazole | 1:30 to 11:1 | 1:10 to 4:1 | 1:4 to 2:1 |
| prochloraz | 1:45 to 5:1 | 1:15 to 2:1 | 1:12 to 1:2 |
| procymidone | 1:90 to 4:1 | 1:30 to 2:1 | 1:18 to 1:2 |
| propamocarb | 1:60 to 3:1 | 1:20 to 1:2 | 1:16 to 1:3 |
| propamocarb-hydrochloride | 1:60 to 3:1 | 1:20 to 1:2 | 1:16 to 1:3 |
| propiconazole | 1:9 to 21:1 | 1:3 to 7:1 | 1:2 to 3:1 |
| propineb | 1:90 to 3:1 | 1:30 to 1:2 | 1:18 to 1:3 |
| proquinazid | 1:6 to 42:1 | 1:2 to 14:1 | 1:1 to 8:1 |
| prothioconazole | 1:60 to 3:1 | 1:20 to 1:2 | 1:16 to 1:3 |
| pyraclostrobin | 1:12 to 21:1 | 1:4 to 7:1 | 1:3 to 3:1 |
| pyrazophos | 1:18 to 21:1 | 1:6 to 7:1 | 1:3 to 3:1 |
| pyribencarb | 1:300 to 42:1 | 1:100 to 14:1 | 1:24 to 1:3 |
| pyrifenox | 1:150 to 3:1 | 1:50 to 1:2 | 1:24 to 1:3 |
| pyrimethanil | 1:30 to 11:1 | 1:10 to 4:1 | 1:4 to 2:1 |
| pyroquilon | 1:60 to 7:1 | 1:20 to 3:1 | 1:6 to 2:1 |
| pyrrolnitrin | 1:30 to 11:1 | 1:10 to 4:1 | 1:4 to 2:1 |
| quinmethionate | 1:300 to 42:1 | 1:100 to 14:1 | 1:24 to 1:3 |
| quinoxyfen | 1:9 to 21:1 | 1:3 to 7:1 | 1:1 to 4:1 |
| quintozene | 1:300 to 42:1 | 1:100 to 14:1 | 1:24 to 1:3 |
| silthiofam | 1:15 to 21:1 | 1:5 to 7:1 | 1:3 to 3:1 |
| simeconazole | 1:30 to 42:1 | 1:10 to 14:1 | 1:3 to 3:1 |
| spiroxamine | 1:45 to 5:1 | 1:15 to 2:1 | 1:9 to 1:2 |
| streptomycin | 1:30 to 11:1 | 1:10 to 4:1 | 1:6 to 2:1 |
| sulfur | 1:600 to 1:3 | 1:200 to 1:8 | 1:120 to 1:14 |
| tebuconazole | 1:15 to 21:1 | 1:5 to 7:1 | 1:3 to 3:1 |
| tecloftalam | 1:300 to 42:1 | 1:100 to 14:1 | 1:24 to 1:3 |
| tecnazene | 1:300 to 42:1 | 1:100 to 14:1 | 1:24 to 1:3 |
| terbinafine | 1:300 to 42:1 | 1:100 to 14:1 | 1:24 to 1:3 |
| tetraconazole | 1:30 to 42:1 | 1:10 to 14:1 | 1:3 to 3:1 |
| thiabendazole | 1:90 to 5:1 | 1:30 to 2:1 | 1:18 to 1:2 |
| thifluzamide | 1:30 to 11:1 | 1:10 to 4:1 | 1:4 to 2:1 |
| thiophanate | 1:90 to 3:1 | 1:30 to 1:2 | 1:18 to 1:3 |
| thiophanate-methyl | 1:90 to 3:1 | 1:30 to 1:2 | 1:18 to 1:3 |
| thiram | 1:300 to 3:1 | 1:100 to 1:2 | 1:60 to 1:7 |
| tiadinil | 1:24 to 11:1 | 1:8 to 4:1 | 1:3 to 2:1 |
| tolclofos-methyl | 1:300 to 3:1 | 1:100 to 1:2 | 1:60 to 1:7 |
| tolylfluanid | 1:300 to 42:1 | 1:100 to 14:1 | 1:24 to 1:3 |
| triadimefon | 1:30 to 42:1 | 1:10 to 14:1 | 1:3 to 3:1 |
| triadimenol | 1:30 to 42:1 | 1:10 to 14:1 | 1:3 to 3:1 |
| triazoxide | 1:300 to 42:1 | 1:100 to 14:1 | 1:24 to 1:3 |
| tricyclazole | 1:30 to 11:1 | 1:10 to 4:1 | 1:4 to 2:1 |
| tridemorph | 1:60 to 4:1 | 1:20 to 2:1 | 1:12 to 1:2 |
| trifloxystrobin | 1:12 to 21:1 | 1:4 to 7:1 | 1:3 to 3:1 |
| triflumizole | 1:30 to 11:1 | 1:10 to 4:1 | 1:4 to 2:1 |
| triforine | 1:30 to 11:1 | 1:10 to 4:1 | 1:4 to 2:1 |
| trimorphamide | 1:90 to 11:1 | 1:30 to 4:1 | 1:12 to 1:2 |
| triticonazole | 1:30 to 42:1 | 1:10 to 14:1 | 1:3 to 3:1 |
| uniconazole | 1:30 to 42:1 | 1:10 to 14:1 | 1:3 to 3:1 |
| validamycin | 1:300 to 42:1 | 1:100 to 14:1 | 1:4 to 2:1 |
| valiphenal | 1:12 to 21:1 | 1:4 to 7:1 | 1:3 to 3:1 |
| vinclozolin | 1:240 to 3:1 | 1:80 to 1:2 | 1:24 to 1:3 |
| zineb | 1:300 to 3:1 | 1:100 to 1:2 | 1:60 to 1:7 |
| ziram | 1:300 to 3:1 | 1:100 to 1:2 | 1:60 to 1:7 |
| zoxamide | 1:12 to 21:1 | 1:4 to 7:1 | 1:3 to 3:1 |
| 5-chloro-6-(2,4,6-trifluorophenyl)-7-(4- | 1:18 to 21:1 | 1:6 to 7:1 | 1:3 to 3:1 |
| methylpiperidin-1-yl)[1,2,4]triazolo[1,5- | | | |
| a]pyrimidine | | | |
| 2-[[[3-(2,6-dichlorophenyl)-1-methyl-2- | 1:6 to 42:1 | 1:2 to 14:1 | 1:1 to 8:1 |
| propen-1-ylidene]amino]oxy]methyl]-a- | | | |
| (methoxyimino)-N-methylbenzeneacetamide | | | |
| 2-butoxy-6-iodo-3-propyl-4H-1-benzopyran-4- | 1:30 to 11:1 | 1:10 to 4:1 | 1:4 to 2:1 |
| one | | | |
| 3-[5-(4-chlorophenyl)-2,3-dimethyl-3- | 1:12 to 21:1 | 1:4 to 7:1 | 1:3 to 3:1 |
| isoxazolidinyl]pyridine | | | |
| 4-fluorophenyl N-[1-[[[1-(4- | 1:30 to 42:1 | 1:10 to 14:1 | 1:2 to 4:1 |
| cyanophenyl)ethyl]sulfonyl]methyl]propyl]carbamate | | | |
| 5-chloro-6-(2,4,6-trifluorophenyl)-7-(4- | 1:18 to 21:1 | 1:6 to 7:1 | 1:4 to 2:1 |
| methylpiperidin-1-yl)[1,2,4]triazolo[1,5- | | | |
| a]pyrimidine | | | |
| α-[methoxyimino]-N-methyl-2-[[[1-[3- | 1:30 to 21:1 | 1:10 to 7:1 | 1:4 to 2:1 |
| (trifluoromethyl)phenyl]ethoxy]imino]methyl]benzeneacetamide | | | |
| N-(4-chloro-2-nitrophenyl)-N-ethyl-4- | 1:3 to 105:1 | 1:1 to 35:1 | 1:1 to 15:1 |
| methylbenzenesulfonamide | | | |
| N-[[(cyclopropylmethoxy)amino][6- | 1:24 to 11:1 | 1:8 to 4:1 | 1:3 to 2:1 |
| (difluoromethoxy)-2,3- | | | |
| difluorophenyl]methylene]benzeneacetamide | | | |
| N-[2-(1,3-dimethylbutyl)phenyl]-5-fluoro-1,3- | 1:24 to 11:1 | 1:8 to 4:1 | 1:3 to 2:1 |
| dimethyl-1H-pyrazol-4-carboxamide | | | |
| N-[2-(1S,2R)-[1,1′-bicyclopropyl]-2-ylphenyl]- | 1:12 to 21:1 | 1:4 to 7:1 | 1:3 to 3:1 |
| 3-(difluoromethyl)-1-methyl-1H-pyrazole-4- | | | |
| carboxamide | | | |
| N-[2-[4-[[3-(4-chlorophenyl)-2-propyn-1- | 1:12 to 21:1 | 1:4 to 7:1 | 1:3 to 3:1 |
| yl]oxy]-3-methoxyphenyl]ethyl]-3-methyl-2- | | | |
| [(methylsulfonyl)amino]butanamide | | | |
| N-[2-[4-[[3-(4-chlorophenyl)-2-propyn-1- | 1:30 to 21:1 | 1:10 to 7:1 | 1:4 to 2:1 |
| yl]oxy]-3-methoxyphenyl]ethyl]-3-methyl-2- | | | |
| [(ethylsulfonyl)amino]butanamide | | | |
| N′-[4-[4-chloro-3-(trifluoromethyl)phenoxy]- | 1:18 to 21:1 | 1:6 to 7:1 | 1:3 to 3:1 |
| 2,5-dimethylphenyl]-N-ethyl-N- | | | |
| methylmethanimidamide | | | |

Examples of insecticides with which compounds of this invention can be formulated are: abamectin, acephate, acetamiprid, amidoflumet (S-1955), avermectin, azadirachtin, azinphos-methyl, bifenthrin, bifenazate, 3-bromo-1-(3-chloro-2-pyridinyl)-N-[4-cyano-2-methyl-6-[(methylamino)carbonyl]phenyl]-1H-pyrazole-5-carboxamide, buprofezin, carbofuran, cartap, chlorantraniliprole (DPX-E2Y45), chlorfenapyr, chlorfluazuron, chlorpyrifos, chlorpyrifos-methyl, chromafenozide, clothianidin, cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim (UR-50701), flufenoxuron, fonophos, halofenozide, hexaflumuron, hydramethylnon, imidacloprid, indoxacarb, isofenphos, lufenuron, malathion, metaflumizone, metaldehyde, methamidophos, methidathion, methomyl, methoprene, methoxychlor, metofluthrin, monocrotophos, methoxyfenozide, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriprole, pyriproxyfen, rotenone, ryanodine, spinetoram, spinosad, spirodiclofen, spiromesifen (BSN 2060), spirotetramat, sulprofos, tebufenozide, teflubenzuron, tefluthrin, terbufos, tetrachlorvinphos, thiacloprid, thiamethoxam, thiodicarb, thiosultap-sodium, tralomethrin, triazamate, trichlorfon and triflumuron; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Table A2 lists specific combinations of invertebrate pest control agents with a compound(s) of Formula 1 illustrative of mixtures and compositions comprising these active ingredients and methods using them according to the present invention. The first column of Table A2 lists the specific invertebrate pest control agents (e.g., “Abamectin” in the first line). The second column of Table A2 lists the mode of action (if known) or chemical class of the invertebrate pest control agents. The third column of Table A2 lists embodiment(s) of ranges of weight ratios for rates at which the invertebrate pest control agent is typically applied relative to a compound(s) of Formula 1. Thus, for example, the first line of Table A2 specifically discloses the combination of a compound(s) of Formula 1 with abamectin is typically applied in a weight ratio between 50:1 to 1:50. The remaining lines of Table A2 are to be construed similarly.

TABLE A2

| Invertebrate Pest | | Typical |
|---|---|---|
| Control Agent | Mode of Action or Chemical Class | Weight Ratio |
| Abamectin | macrocyclic lactones | 50:1 to 1:50 |
| Acetamiprid | neonicotinoids | 150:1 to 1:200 |
| Amitraz | octopamine receptor ligands | 200:1 to 1:100 |
| Avermectin | macrocyclic lactones | 50:1 to 1:50 |
| Azadirachtin | ecdysone agonists | 100:1 to 1:120 |
| Beta-cyfluthrin | sodium channel modulators | 150:1 to 1:200 |
| Bifenthrin | sodium channel modulators | 100:1 to 1:10 |
| Buprofezin | chitin synthesis inhibitors | 500:1 to 1:50 |
| Cartap | nereistoxin analogs | 100:1 to 1:200 |
| Chlorantraniliprole | ryanodine receptor ligands | 100:1 to 1:120 |
| Chlorfenapyr | mitochondrial electron transport inhibitors | 300:1 to 1:200 |
| Chlorpyrifos | cholinesterase inhibitors | 500:1 to 1:200 |
| Clothianidin | neonicotinoids | 100:1 to 1:400 |
| Cyfluthrin | sodium channel modulators | 150:1 to 1:200 |
| Cyhalothrin | sodium channel modulators | 150:1 to 1:200 |
| Cypermethrin | sodium channel modulators | 150:1 to 1:200 |
| Cyromazine | chitin synthesis inhibitors | 400:1 to 1:50 |
| Deltamethrin | sodium channel modulators | 50:1 to 1:400 |
| Dieldrin | cyclodiene insecticides | 200:1 to 1:100 |
| Dinotefuran | neonicotinoids | 150:1 to 1:200 |
| Diofenolan | molting inhibitor | 150:1 to 1:200 |
| Emamectin | macrocyclic lactones | 50:1 to 1:10 |
| Endosulfan | cyclodiene insecticides | 200:1 to 1:100 |
| Esfenvalerate | sodium channel modulators | 100:1 to 1:400 |
| Ethiprole | GABA-regulated chloride channel blockers | 200:1 to 1:100 |
| Fenothiocarb | | 150:1 to 1:200 |
| Fenoxycarb | juvenile hormone mimics | 500:1 to 1:100 |
| Fenvalerate | sodium channel modulators | 150:1 to 1:200 |
| Fipronil | GABA-regulated chloride channel blockers | 150:1 to 1:100 |
| Flonicamid | | 200:1 to 1:100 |
| Flubendiamide | ryanodine receptor ligands | 100:1 to 1:120 |
| Flufenoxuron | chitin synthesis inhibitors | 200:1 to 1:100 |
| Hexaflumuron | chitin synthesis inhibitors | 300:1 to 1:50 |
| Hydramethylnon | mitochondrial electron transport inhibitors | 150:1 to 1:250 |
| Imidacloprid | neonicotinoids | 1000:1 to 1:1000 |
| Indoxacarb | sodium channel modulators | 200:1 to 1:50 |
| Lambda-cyhalothrin | sodium channel modulators | 50:1 to 1:250 |
| Lufenuron | chitin synthesis inhibitors | 500:1 to 1:250 |
| Metaflumizone | | 200:1 to 1:200 |
| Methomyl | cholinesterase inhibitors | 500:1 to 1:100 |
| Methoprene | juvenile hormone mimics | 500:1 to 1:100 |
| Methoxyfenozide | ecdysone agonists | 50:1 to 1:50 |
| Nitenpyram | neonicotinoids | 150:1 to 1:200 |
| Nithiazine | neonicotinoids | 150:1 to 1:200 |
| Novaluron | chitin synthesis inhibitors | 500:1 to 1:150 |
| Oxamyl | cholinesterase inhibitors | 200:1 to 1:200 |
| Pymetrozine | | 200:1 to 1:100 |
| Pyrethrin | sodium channel modulators | 100:1 to 1:10 |
| Pyridaben | mitochondrial electron transport inhibitors | 200:1 to 1:100 |
| Pyridalyl | | 200:1 to 1:100 |
| Pyriproxyfen | juvenile hormone mimics | 500:1 to 1:100 |
| Ryanodine | ryanodine receptor ligands | 100:1 to 1:120 |
| Spinetoram | macrocyclic lactones | 150:1 to 1:100 |
| Spinosad | macrocyclic lactones | 500:1 to 1:10 |
| Spirodiclofen | lipid biosynthesis inhibitors | 200:1 to 1:200 |
| Spiromesifen | lipid biosynthesis inhibitors | 200:1 to 1:200 |
| Tebufenozide | ecdysone agonists | 500:1 to 1:250 |
| Thiacloprid | neonicotinoids | 100:1 to 1:200 |
| Thiamethoxam | neonicotinoids | 1250:1 to 1:1000 |
| Thiodicarb | cholinesterase inhibitors | 500:1 to 1:400 |
| Thiosultap-sodium | | 150:1 to 1:100 |
| Tralomethrin | sodium channel modulators | 150:1 to 1:200 |
| Triazamate | cholinesterase inhibitors | 250:1 to 1:100 |
| Triflumuron | chitin synthesis inhibitors | 200:1 to 1:100 |
| Bacillus thuringiensis | biological agents | 50:1 to 1:10 |
| Bacillus thuringiensis delta- | biological agents | 50:1 to 1:10 |
| endotoxin | | |
| NPV (e.g., Gemstar) | biological agents | 50:1 to 1:10 |

One embodiment of invertebrate pest control agents (e.g., insecticides and acaricides) for mixing with a compound(s) of Formula 1 include sodium channel modulators such as bifenthrin, cypermethrin, cyhalothrin, lambda-cyhalothrin, cyfluthrin, beta-cyfluthrin, deltamethrin, dimefluthrin, esfenvalerate, fenvalerate, indoxacarb, metofluthrin, profluthrin, pyrethrin and tralomethrin; cholinesterase inhibitors such as chlorpyrifos, methomyl, oxamyl, thiodicarb and triazamate; neonicotinoids such as acetamiprid, clothianidin, dinotefuran, imidacloprid, nitenpyram, nithiazine, thiacloprid and thiamethoxam; insecticidal macrocyclic lactones such as spinetoram, spinosad, abamectin, avermectin and emamectin; GABA (γ-aminobutyric acid)-regulated chloride channel blockers such as endosulfan, ethiprole and fipronil; chitin synthesis inhibitors such as buprofezin, cyromazine, flufenoxuron, hexaflumuron, lufenuron, novaluron, noviflumuron and triflumuron; juvenile hormone mimics such as diofenolan, fenoxycarb, methoprene and pyriproxyfen; octopamine receptor ligands such as amitraz; ecdysone agonists such as azadirachtin, methoxyfenozide and tebufenozide; ryanodine receptor ligands such as ryanodine, anthranilic diamides such as chlorantraniliprole (see U.S. Pat. No. 6,747,047, PCT Publications WO 2003/015518 and WO 2004/067528), flubendiamide (see U.S. Pat. No. 6,603,044), 3-bromo-1-(3-chloro-2-pyridinyl)-N-[4-cyano-2-methyl-6-[[(1-methylethyl)amino]carbonyl]phenyl]-1H-pyrazole-5-carboxamide, 3-bromo-1-(3-chloro-2-pyridinyl)-N-[4-cyano-2-methyl-6-[(methylamino)carbonyl]phenyl]-1H-pyrazole-5-carboxamide, 3-chloro-1-(3-chloro-2-pyridinyl)-N-[4-cyano-2-methyl-6-[(methylamino)carbonyl]phenyl]-1H-pyrazole-5-carboxamide and 3-chloro-1-(3-chloro-2-pyridinyl)-N-[4-cyano-2-methyl-6-[[(1-methylethyl)amino]carbonyl]phenyl]-1H-pyrazole-5-carboxamide; nereistoxin analogs such as cartap; mitochondrial electron transport inhibitors such as chlorfenapyr, hydramethylnon and pyridaben; lipid biosynthesis inhibitors such as spirodiclofen and spiromesifen; cyclodiene insecticides such as dieldrin; cyflumetofen; fenothiocarb; flonicamid; metaflumizone; pyrafluprole; pyridalyl; pyriprole; pymetrozine; spirotetramat; and thiosultap-sodium. One embodiment of biological agents for mixing with a compound(s) Formula 1 include nucleopolyhedro virus such as HzNPV and AfNPV; Bacillus thuringiensis and encapsulated delta-endotoxins of Bacillus thuringiensis such as Cellcap, MPV and MPVII; as well as naturally occurring and genetically modified viral insecticides including members of the family Baculoviridae as well as entomophagous fungi. Of note is a composition comprising a compound of Formula 1 and at least one additional biologically active compound or agent selected from the Invertebrate Pest Control Agents listed in Table A2 above.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

General references for these agricultural protectants (i.e. insecticides, fungicides, nematocides, acaricides, herbicides and biological agents) include The Pesticide Manual, 13^{th} Edition, C. D. S. Tomlin, Ed., British Crop Protection Council, Farnham, Surrey, U.K., 2003 and The BioPesticide Manual, 2^{nd} Edition, L. G. Copping, Ed., British Crop Protection Council, Farnham, Surrey, U.K., 2001.

Compounds of this invention and mixtures with one or more other biologically active compounds provide control of diseases caused by a broad spectrum of fungal plant pathogens in the Basidiomycete, Ascomycete, Oomycete and Deuteromycete classes. They are effective in controlling a broad spectrum of plant diseases, foliar pathogens of crops including: cereal grain crops such as wheat, barley, oats, rye, triticale, rice, maize, sorghum and millet; vine crops such as table and wine grapes; field crops such as oilseed rape (canola), sunflower; sugar beets, sugar cane, soybean, peanuts (groundnut), tobacco, alfafa, clover, lespedeza, trefoil and vetch; pome fruits such as apple, pear, crabapple, loquat, mayhaw and quince; stone fruits such as peaches, cherries, plums, apricots, nectarines and almonds; citrus fruits such as lemons, limes, oranges, grapefruit, mandarin (tangerines) and kumquat; root and tuber vegetables and field crops (and their foliage) such as artichoke, garden and sugar beet, carrot, cassaya, ginger, ginseng, horseradish, parsnip, potato, radish, rutabaga, sweet potato, turnip and yam; bulb vegetables such as garlic, leek, onion and shallot; leafy vegetables such as arugula (roquette), celery, celery, cress, endive (escarole), fennel, head and leaf lettuce, parsley, radicchio (red chicory), rhubarb, spinach and Swiss chard; brassica (cole) leafy vegetables such as broccoli, broccoli raab (rapini), Brussels sprouts, cabbage, bok Choy, cauliflower, collards, kale, kohlrabi, mustard and greens; legume vegetables (succulent or dried) such as lupin, bean (Phaseolus spp.) (including field bean, kidney bean, lima bean, navy bean, pinto bean, runner bean, snap bean, tepary bean and wax bean), bean (Vigna spp.) (including adzuki bean, asparagus bean, blackeyed pea, catjang, Chinese longbean, cowpea, crowder pea, moth bean, mung bean, rice bean, southern pea, urd bean and yardlong bean), broad bean (fava), chickpea (garbanzo), guar, jackbean, lablab bean, lentil and pea (Pisum spp.) (including dwarf pea, edible-podded pea, English pea, field pea, garden pea, green pea, snowpea, sugar snap pea, pigeon pea and soybean); fruiting vegetables such as eggplant, groundcherry (Physalis spp.), pepino and pepper (including bell pepper, chili pepper, cooking pepper, pimento, sweet pepper; tomatillo and tomato); cucurbit vegetables such as Chayote (fruit), Chinese waxgourd (Chinese preserving melon), citron melon, cucumber, gherkin, edible gourd (including hyotan, cucuzza, hechima, and Chinese okra), Momordica spp. (including balsam apple, balsam pear, bittermelon and Chinese cucumber), muskmelon (including cantaloupe and pumpkin), summer and winter squash (including butternut squash, calabaza, hubbard squash, acorn squash, spaghetti squash) and watermelon; berries such as blackberry (including bingleberry, boysenberry, dewberry, lowberry, marionberry, olallieberry and youngberry), blueberry, cranberry, currant, elderberry, gooseberry, huckleberry, loganberry, raspberry and strawberry; tree nuts such as almond, beech nut, Brazil nut, butternut, cashew, chestnut, chinquapin, filbert (hazelnut), hickory nut, macadamia nut, pecan and walnut; tropical fruits and other crops such as bananas, plantains, mangos, coconuts, papaya, guava, avocado, lichee, agave, coffee, cacao, sugar cane, oil palm, sesame, rubber and spices; fiber crops such as cotton, flax and hemp; turfgrasses (including warm- and cool-season turfgrasses) such as bentgrass, Kentucky bluegrass, St. Augustine grass, tall fescue and Bermuda grass.

These pathogens include: Oomycetes, including Phytophthora diseases such as Phytophthora infestans, Phytophthora megasperma, Phytophthora parasitica, Phytophthora cinnamomi and Phytophthora capsici, Pythium diseases such as Pythium aphanidermatum, and diseases in the Peronosporaceae family such as Plasmopara viticola, Peronospora spp. (including Peronospora tabacina and Peronospora parasitica), Pseudoperonospora spp. (including Pseudoperonospora cubensis) and Bremia lactucae; Ascomycetes, including Alternaria diseases such as Alternaria solani and Alternaria brassicae, Guignardia diseases such as Guignardia bidwelli, Venturia diseases such as Venturia inaequalis, Septoria diseases such as Septoria nodorum and Septoria tritici, powdery mildew diseases such as Erysiphe spp. (including Erysiphe graminis and Erysiphe polygoni), Uncinula necatur, Sphaerotheca fuligena and Podosphaera leucotricha, Pseudocercosporella herpotrichoides, Botrytis diseases such as Botrytis cinerea, Monilinia fructicola, Sclerotinia diseases such as Sclerotinia sclerotiorum, Magnaporthe grisea, Phomopsis viticola, Helminthosporium diseases such as Helminthosporium tritici repentis, Pyrenophora teres, anthracnose diseases such as Glomerella or Colletotrichum spp. (such as Colletotrichum graminicola and Colletotrichum orbiculare), and Gaeumannomyces graminis; Basidiomycetes, including rust diseases caused by Puccinia spp. (such as Puccinia recondite, Puccinia striiformis, Puccinia hordei, Puccinia graminis and Puccinia arachidis), Hemileia vastatrix and Phakopsora pachyrhizi; other pathogens including Rhizoctonia spp. (such as Rhizoctonia solani and Rhizoctonia oryzae); Fusarium diseases such as Fusarium roseum, Fusarium graminearum and Fusarium oxysporum; Verticillium dahliae; Sclerotium rolfsii; Rynchosporium secalis; Cercosporidium personatum, Cercospora arachidicola and Cercospora beticola; Rutstroemia floccosum (also known as Sclerontina homoeocarpa); and other genera and species closely related to these pathogens. In addition to their fungicidal activity, the compositions or combinations also have activity against bacteria such as Erwinia amylovora, Xanthomonas campestris, Pseudomonas syringae, and other related species.

Mixtures of fungicides may provide significantly better disease control than could be predicted based on the activity of the individual components. This synergism has been described as “the cooperative action of two components of a mixture, such that the total effect is greater or more prolonged than the sum of the effects of the two (or more) taken independently” (see Tames, P. M. L., Neth. J. Plant Pathology, (1964), 70, 73-80).

Specifically preferred mixtures (compound numbers refer to compounds in Index Tables A-C) are selected from the group: combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with azoxystrobin, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with kresoxim-methyl, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with trifloxystrobin, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with picoxystrobin, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with metominostrobin/fenominostrobin, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with quinoxyfen, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with metrafenone, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with fenpropidine, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with fenpropimorph, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with cyproconazole, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with epoxiconazole, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with flusilazole, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with metconazole, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with propiconazole, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with proquinazid, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with prothioconazole, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with tebuconazole, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with triticonazole, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with famoxadone, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with penthiopyrad, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with 3-(difluoromethyl)-1-methyl-N-(3′,4′,5′-trifluoro[1,1′-biphenyl]-2-yl)-1H-pyrazole-4-carboxamide, combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with 5-ethyl-6-octyl-[1,2,4]triazole[1,5-a]pyrimidin-7-amine, and combinations of Compound 2, Compound 7, Compound 8, Compound 9, Compound 10, Compound 11, Compound 37, Compound 122, Compound 159, Compound 205, Compound 280, Compound 393, Compound 406, Compound 430, Compound 499, Compound 500, Compound 553 with Initium®.

The following TESTS demonstrate the control efficacy of compounds of this invention on specific pathogens. The pathogen control protection afforded by the compounds is not limited, however, to these species. See Index Tables A-C for compound descriptions. See Index Table D for ¹H NMR data. The following abbreviations are used in the Index Tables which follow: i means iso, Me is methyl, Et is ethyl, Ph is phenyl, Bn is benzyl, MeO is methoxy, EtO is ethoxy, MeS is methylthio and CN is cyano. In the Index Tables when an instance of Q¹, Q² and Q³ comprises a phenyl ring attached through the linker CR^{7a}R^{7b} to the remained of Formula 1, locant numbers of the ring are relative to the connection of the ring to the linker CR^{7a}R^{7b}. The abbreviation “Cmpd.” stands for “Compound”, and the abbreviation “Ex.” stands for “Example” and is followed by a number indicating in which example the compound is prepared. The abbreviation “m.p.” stands for melting point. In Index Tables A-C the numerical value reported in the column “AP⁺ (M+1)”, is the molecular weight of the observed molecular ion formed by addition of H⁺ (molecular weight of 1) to the molecule having the greatest isotopic abundance (i.e. M). The presence of molecular ions containing one or more higher atomic weight isotopes of lower abundance (e.g., ³⁷C, 81c) is not reported. The reported M+1 peaks were observed by mass spectrometry using atmospheric pressure chemical ionization (AP⁺).

INDEX TABLE A

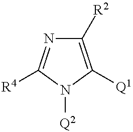

| Cmpd. | Q¹ | Q² | R² | R⁴ | m.p. (° C.) | AP⁺ (M + 1) |
|---|---|---|---|---|---|---|
| 1 (Ex. 1) | 2,4,6-tri-F—Ph | 4-Cl—Ph | Cl | H | *** | *** |
| 2 (Ex. 5) | 2,6-di-F—Ph | 4-Cl—Ph | Cl | Me | *** | *** |
| 3 (Ex. 3) | 2,6-di-F—Ph | 4-Cl—Ph | Cl | Cl | *** | *** |
| 4 (Ex. 7) | 4-F—Ph | 4-Cl—Ph | H | Me | *** | *** |
| 5 (Ex. 8) | 4-F—Ph | 4-Cl—Ph | Cl | Me | *** | *** |
| 6 (Ex. 2) | 2,4,6-tri-F—Ph | 4-Cl—Ph | Cl | Cl | *** | *** |
| 7 | 4-Cl—Ph | 2-Cl, 4-F—Ph | Cl | Cl | 129-131 | |
| 9 | 2,6-di-F—Ph | Ph | Cl | H | | 291 |
| 10 | 2,6-di-F—Ph | Ph | Cl | Br | | 371 |
| 11 | 2,4,6-tri-F—Ph | 4-Cl—Ph | Cl | Br | 139-141 | |
| 12 | 2,4,6-tri-F—Ph | 4-Cl—Ph | Cl | Me | 148-150 | |
| 13 | 2,6-di-F—Ph | Ph | Cl | I | | 417 |
| 14 | 2,6-di-F—Ph | 3-F—Ph | Cl | H | 112-116 | |
| 15 | 2,6-di-F—Ph | Ph | Cl | Me | | 305 |
| 16 | 2,6-di-F, 4-MeO—Ph | 4-Cl, 3-F—Ph | Me₂N | H | | 382 |
| 17 | 2,6-di-F, 4-MeO—Ph | 4-Cl, 3-F—Ph | Cl | H | | 373 |
| 18 | 2,4,6-tri-F—Ph | 4-Cl, 3-F—Ph | Cl | H | 114-117 | |
| 19 | 2,6-di-F—Ph | 3-F—Ph | Cl | Cl | 131-134 | |
| 20 | 2,4,6-tri-F—Ph | 4-Cl—Ph | Br | H | 68-70 | |
| 21 | 2,4,6-tri-F—Ph | 4-Cl—Ph | Br | Br | 145-148 | |
| 22 | 2,6-di-F—Ph | 3-F—Ph | Cl | Br | 120-122 | |
| 23 | 2,4,6-tri-F—Ph | 4-Cl, 3-F—Ph | Cl | Br | 135-137 | |
| 27 | 2,4,6-tri-F—Ph | 4-Cl, 3-F—Ph | Cl | Cl | | 397 |
| 28 | 2,6-di-F—Ph | 3-F—Ph | Cl | Me | | 323 |
| 29 | 2,4,6-tri-F—Ph | 4-Cl, 3-F—Ph | Cl | Me | | 375 |
| 32 | 2,6-di-F, 4-MeO—Ph | 4-Cl, 3-F—Ph | Cl | Br | ** | ** |
| 33 | 2,6-di-F, 4-MeO—Ph | 4-Cl, 3-F—Ph | Br | H | | 417 |
| 34 | 2,6-di-F, 4-MeO—Ph | 4-Cl, 3-F—Ph | Cl | Me | | 387 |
| 35 | 2,6-di-F—Ph | 4-Cl—Ph | Cl | Br | 124-126 | |
| 36 | 2,4,6-tri-F—Ph | 4-Cl, 3-F—Ph | Br | Br | 144-146 | |
| 37 | 2,6-di-F, 4-MeO—Ph | 4-Cl—Ph | Cl | Me | 146-148 | |
| 38 | 2,6-di-F, 4-MeO—Ph | 4-Cl—Ph | Br | H | 128-130 | |
| 39 | 2,6-di-F, 4-MeO—Ph | 4-Cl—Ph | Cl | H | 124-127 | |
| 40 | 2,6-di-F, 4-MeO—Ph | 4-Cl—Ph | Cl | Br | 152-153 | |
| 41 | 2,4,6-tri-F—Ph | 4-Cl, 3-F—Ph | Me | Me | | 355 |
| 42 | 2,6-di-F, 4-MeO—Ph | 4-Cl—Ph | Br | Br | 166-168 | |
| 43 | 2,6-di-F, 4-MeO—Ph | 4-Cl, 3-F—Ph | Br | Br | | 495 |
| 44 | 2,6-di-F, 4-MeO—Ph | 4-Cl, 3-F—Ph | Me | Me | | 367 |
| 45 | 2,4,6-tri-F—Ph | 4-Cl—Ph | Me | Me | ** | ** |
| 46 | 2,6-di-F, 4-MeO—Ph | 4-Cl, 3-F—Ph | Me | H | | 353 |
| 47 (Ex. 19) | 2,6-di-F—Ph | 3-F—Ph | Cl | CH≡C | | 333 |
| 48 | 2,3,6-tri-F—Ph | 4-Cl—Ph | Cl | Me | | 357 |
| 49 | 2-Cl, 4-F—Ph | 4-Cl—Ph | Cl | Me | | 357 |
| 50 | 2,3,6-tri-F—Ph | 4-Cl—Ph | Cl | Br | 124-127 | |
| 51 | 2-Cl, 4-F—Ph | 4-Cl—Ph | Cl | Br | 117-120 | |
| 52 | 2-Cl, 6-F—Ph | 4-Cl—Ph | Cl | Br | 174-177 | |
| 53 | 2-Cl, 6-F—Ph | 4-Cl—Ph | Cl | Me | | 357 |
| 54 | 2,6-di-F—Ph | 3,5-di-F—Ph | Cl | Br | | 407 |
| 55 | 2,6-di-F, 4-MeO—Ph | 4-Cl—Ph | Me | Me | 105-108 | |
| 56 | 2,6-di-F, 4-MeO—Ph | 4-Cl—Ph | Me | H | 98-101 | |
| 57 (Ex. 18) | 2,3,6-tri-F—Ph | 4-Cl—Ph | Cl | CN | 147-149 | |
| 58 | 2,6-di-F—Ph | 2-F—Ph | Cl | Br | 113-115 | |
| 59 | 2,6-di-F, 4-MeO, 3- | 4-Cl—Ph | Br | Me | 143-147 | |
| | Me—Ph | | | | | |
| 64 | 2,6-di-F—Ph | 2-F—Ph | Cl | Me | | 323 |
| 65 | 2,6-di-F—Ph | 4-CF₃O—Ph | Cl | Br | | 355 |
| 66 | 2,6-di-F—Ph | 4-Br—Ph | Cl | Br | 143-145 | |
| 72 | 2,6-di-F—Ph | 4-F—Ph | Cl | Br | | 389 |
| 73 | 2,6-di-F—Ph | 3,5-di-F—Ph | Cl | Me | | 341 |
| 74 | 2-Cl, 3,6-di-F—Ph | 4-Cl—Ph | Cl | H | | 360 |
| 75 | 3-Br, 2,6-di-F—Ph | 4-Cl—Ph | Cl | H | | 405 |
| 76 | 2-Cl, 3,6-di-F—Ph | 4-Cl—Ph | Cl | Br | | 439 |
| 80 | 2-Cl, 3,6-di-F—Ph | 4-Cl—Ph | Cl | Me | | 373 |
| 81 | 2,6-di-F—Ph | 4-CF₃O—Ph | Cl | Me | | 389 |
| 82 | 2,6-di-F—Ph | 4-Me—Ph | Cl | Me | | 319 |
| 83 | 2,6-di-F—Ph | 4-F—Ph | Cl | Me | | 323 |
| 84 | 2,6-di-F—Ph | 4-CF₃—Ph | Cl | Br | 117-120 | |
| 85 | 2,5-di-F—Ph | 4-Cl—Ph | Cl | Br | 114-117 | |
| 86 | 2-CF3, 6-F—Ph | 4-Cl—Ph | Cl | Br | | 455 |
| 90 | 2-Cl, 6-F—Ph | 4-Cl—Ph | Cl | CF3 | | 409 |
| 91 | 3-Br, 2,6-di-F—Ph | 4-Cl—Ph | Cl | Me | | 419 |
| 92 | 2,6-di-F—Ph | 4-CF₃—Ph | Cl | Me | | 373 |
| 93 | 2,5-di-F—Ph | 4-Cl—Ph | Cl | Me | | 339 |
| 94 | 2-CF3, 6-F—Ph | 4-Cl—Ph | Cl | Me | | 389 |
| 95 | 2,6-di-F—Ph | Bn | Cl | Br | 85-87 | |
| 96 | 2,6-di-F, 4-MeO—Ph | 4-Cl—Ph | Br | MeO | 141-144 | |
| 97 | 2,6-di-F, 3-Me—Ph | 4-Cl—Ph | Cl | H | | 339 |
| 98 | 4-Cl—Ph | 2,4,6-tri-F—Ph | Cl | H | 130-131 | |
| 99 | 2,6-di-F—Ph | 4-Cl—Bn | Me2N | H | | 348 |
| 100 | 3-Cl, 2, 6-di-F—Ph | 4-Cl—Ph | Cl | Br | | 439 |
| 101 | 2,6-di-F—Ph | 3-Cl—Bn | Cl | Br | | 419 |
| 102 | 2,6-di-F—Ph | 4-Cl, 3-F—Bn | Cl | Br | | 437 |
| 103 | 2,6-di-F—Ph | 4-C—Bn | Cl | Br | | 419 |
| 105 | 2,6-di-F, 3-Me—Ph | 4-Cl—Ph | Cl | Br | | 419 |
| 106 | 2-F, 4-MeO—Ph | 4-Cl—Ph | Cl | H | | 337 |
| 107 | 2,6-di-F, 3-Me—Ph | 4-Cl—Ph | Cl | Me | | 353 |
| 108 | 2,6-di-F—Ph | 4-Cl—Ph | Cl | Et | 125-126 | |
| 109 | 3-Br, 2-F, 4-MeO—Ph | 4-Cl—Ph | Cl | Br | | 498 |
| 110 | 2-F, 4-MeO—Ph | 4-Cl—Ph | Cl | Br | | 417 |
| 112 | 2,6-di-F, 3-MeO—Ph | 4-Cl—Ph | Cl | H | | 356 |
| 113 | 2-F, 4-MeO—Ph | 4-Cl—Ph | Cl | Me | | 351 |
| 114 | 2-F, 4-MeO, 3-Me—Ph | 4-Cl—Ph | Cl | Me | | 365 |
| 116 | 2,6-di-F—Ph | 4-Cl—Ph | Cl | i-Pr | 106-108 | |
| 117 | 2,6-di-F, 3-MeO—Ph | 4-Cl—Ph | Cl | Br | | 435 |
| 118 | 2,6-di-F—Ph | Bn | Cl | Me | | 319 |
| 119 | 2,6-di-F, 4-HO—Ph | 4-Cl—Ph | Cl | Me | | 355 |
| 120 | 2,6-di-F—Ph | 4-Cl, 3-F—Bn | Cl | Me | | 371 |
| 121 | 2,3,6-tri-F—Ph | 4-F—Ph | Cl | H | | 327 |
| 122 | 2,6-di-F, 3-MeO—Ph | 4-Cl—Ph | Cl | Me | | 369 |
| 123 | 2,3,6-tri-F—Ph | 4-Me—Ph | Cl | H | | 323 |
| 124 | 2,3,6-tri-F—Ph | 4-Cl, 3-F—Ph | Cl | H | | 361 |
| 126 | 2,6-di-F—Ph | 3-Cl—Bn | Cl | Me | | 353 |
| 127 | 3-Cl, 2,6-di-F—Ph | 4-Cl—Ph | Cl | Me | | 375 |
| 128 | 2,6-di-F—Ph | 4-Cl—Bn | Cl | Me | | 353 |
| 129 | 2,3,6-tri-F—Ph | 4-F—Ph | Cl | Cl | | 361 |
| 130 | 2,3,6-tri-F—Ph | 4-Me—Ph | Cl | Cl | | 357 |
| 131 | 4-Cl—Ph | 2,4,6-tri-F—Ph | Cl | Br | 117-118 | |
| 132 | 2-Cl, 6-F, 4-MeO—Ph | 4-Cl—Ph | Cl | H | | 371 |
| 133 | 4-Cl, 2-F, 6-MeO—Ph | 4-Cl—Ph | Cl | H | | 371 |
| 134 | 2,3,6-tri-F—Ph | 4-F—Ph | Cl | Br | | 407 |
| 135 | 2,3,6-tri-F—Ph | 4-Cl, 3-F—Ph | Cl | Cl | | 395 |
| 136 | 2,6-di-F, 4-MeO—Ph | 4-F—Ph | Cl | H | | 339 |
| 137 | 2,4,6-tri-F—Ph | 4-F—Ph | Cl | H | | 327 |
| 138 | 2,6-di-F, 4-MeO—Ph | 4-Me—Ph | Cl | H | | 335 |
| 139 | 2,4,6-tri-F—Ph | 4-Me—Ph | Cl | H | | 323 |
| 140 | 4-Cl, 2-F, 6-MeO—Ph | 4-Cl—Ph | Cl | Br | | 451 |
| 141 | 2,3,6-tri-F—Ph | 4-Me—Ph | Cl | Br | | 403 |
| 142 | 2,6-di-F, 4-MeO—Ph | 4-F—Ph | Cl | Cl | | 373 |
| 143 | 2,3,6-tri-F—Ph | 4-Cl, 3-F—Ph | Cl | Br | | 441 |
| 144 | 2,6-di-F, 4-MeO—Ph | 4-F—Ph | Cl | Br | | 419 |
| 145 | 2,3,6-tri-F—Ph | 4-F—Ph | Cl | Me | | 341 |
| 146 | 2,6-di-F, 4-MeO—Ph | 4-Me—Ph | Cl | Cl | | 369 |
| 147 | 2,4,6-tri-F—Ph | 4-F—Ph | Cl | Cl | | 361 |
| 148 | 2,6-di-F, 4-MeO—Ph | 4-Me—Ph | Cl | Br | | 415 |
| 149 | 4-Cl, 2-F, 6-MeO—Ph | 4-Cl—Ph | Cl | Me | | 387 |
| 150 | 4-Cl—Ph | 2,6-di-F—Ph | Cl | Cl | 123-126 | |
| 151 | 2,6-di-F—Ph | 4-Cl—Ph | Me | Me | 146-148 | |
| 152 | 2,4,6-tri-F—Ph | 4-F—Ph | Cl | Br | | 407 |
| 153 | 2,4,6-tri-F—Ph | 4-Me—Ph | Cl | Br | | 403 |
| 154 | 2,4,6-tri-F—Ph | 4-Me—Ph | Cl | Cl | | 357 |
| 155 | 2,3,6-tri-F—Ph | 4-Me—Ph | Cl | Me | | 337 |
| 156 | 4-EtO, 2,6-di-F—Ph | 4-Cl—Ph | Cl | Me | | 383 |
| 157 | 2,3,6-tri-F—Ph | 4-Cl, 3-F—Ph | Cl | Me | | 375 |
| 158 | 2,6-di-F, 4-MeO—Ph | 4-F—Ph | Cl | Me | | 353 |
| 159 | 2,6-di-F, 4-MeO—Ph | 4-Me—Ph | Cl | Me | | 349 |
| 160 | 2,6-di-F—Ph | 3,4-di-F—Ph | Cl | H | | 327 |
| 161 | 2,6-di-F—Ph | 3-F, 4-Me—Ph | Cl | H | | 323 |
| 162 | 4-Cl—Ph | 2,6-di-F—Ph | Br | Br | 156-158 | |
| 163 | 4-Cl—Ph | 2,6-di-F—Ph | Cl | H | 182-184 | |
| 164 | 4-Cl—Ph | 2,6-di-F—Ph | Cl | Br | 148-150 | |
| 167 | 2,4,6-tri-F—Ph | 4-F—Ph | Cl | Me | | 341 |
| 168 | 2,4,6-tri-F—Ph | 4-Me—Ph | Cl | Me | | 337 |
| 169 | 2,6-di-F—Ph | 3,4-di-F—Ph | Cl | Br | | 407 |
| 170 | 2,6-di-F—Ph | 3-F, 4-Me—Ph | Cl | Br | | 403 |
| 171 | 2-Cl, 6-F, 4-MeO—Ph | 4-Cl—Ph | Cl | Br | | 451 |
| 172 | 2-Cl, 6-F, 4-MeO—Ph | 4-Cl—Ph | Cl | Me | | 385 |
| 173 | 4-Cl—Ph | 2,6-di-F, 4-MeO—Ph | Br | H | 123-124 | |
| 174 | 2,6-di-F—Ph | 3,4-di-F—Ph | Cl | Me | | 341 |
| 175 | 2,6-di-F—Ph | 3-F, 4-Me—Ph | Cl | Me | | 337 |
| 176 | 2-Cl, 3,6-di-F—Ph | 4-F—Ph | Cl | H | | 343 |
| 177 | 2-Cl, 3,6-di-F—Ph | 4-Me—Ph | Cl | H | | 339 |
| 178 | 2-Cl, 4-F—Ph | 4-F—Ph | Cl | H | | 325 |
| 179 | 2-Cl, 4-F—Ph | 4-Me—Ph | Cl | H | | 321 |
| 180 | 2-Cl, 6-F—Ph | 4-F—Ph | Cl | H | | 325 |
| 181 | 2-Cl, 6-F—Ph | 4-Me—Ph | Cl | H | | 321 |
| 182 | 2-Cl, 4-F—Ph | 4-F—Ph | Cl | Br | 111-112 | |
| 183 | 2-Cl, 4-F—Ph | 4-F—Ph | Cl | Cl | | 359 |
| 184 | 2-Cl, 4-F—Ph | 4-Me—Ph | Cl | Br | 127-128 | |
| 185 | 2-Cl, 6-F—Ph | 4-F—Ph | Cl | Br | 95-97 | |
| 186 | 2-Cl, 6-F—Ph | 4-Me—Ph | Cl | Br | 147-148 | |
| 187 | 2-Cl, 4-F—Ph | 4-F—Ph | Cl | Me | 101-106 | |
| 188 | 2-Cl, 4-F—Ph | 4-Me—Ph | Cl | Me | 148-149 | |
| 189 | 2-Cl, 6-F—Ph | 4-F—Ph | Cl | Me | 122-123 | |
| 190 | 2-Cl, 6-F—Ph | 4-Me—Ph | Cl | Me | 158-162 | |
| 191 | 2-Cl, 4-F—Ph | 4-Cl, 3-F—Ph | Cl | H | | 359 |
| 192 | 2-Cl, 6-F—Ph | 4-Cl, 3-F—Ph | Cl | H | | 359 |
| 193 | 4-CN, 2,6-di-F—Ph | 4-Cl—Ph | Cl | H | | 350 |
| 194 | 2,6-di-F—Ph | 4-MeO—Bn | Cl | H | | 335 |
| 195 | 2-Cl, 3,6-di-F—Ph | 4-F—Ph | Cl | Br | | 423 |
| 196 | 2-Cl, 3,6-di-F—Ph | 4-F—Ph | Cl | Cl | | 378 |
| 197 | 2-Cl, 3,6-di-F—Ph | 4-Me—Ph | Cl | Br | | 419 |
| 198 | 2-Cl, 3,6-di-F—Ph | 4-Me—Ph | Cl | Cl | | 374 |
| 199 | 2,6-di-F—Ph | 3-quinolinyl | Cl | Me | 158-160 | |
| 200 | 4-Cl—Ph | 2,6-di-F—Ph | Br | H | 195-197 | |
| 201 | 4-CN, 2,6-di-F—Ph | 4-Cl—Ph | Cl | Br | | 429 |
| 202 | 2-Cl, 4-F—Ph | 4-Cl, 3-F—Ph | Cl | Br | 103-104 | |
| 203 | 2-Cl, 6-F—Ph | 4-Cl, 3-F—Ph | Cl | Br | 80-84 | |
| 204 | 4-Cl—Ph | 2,4,6-tri-F—Ph | Cl | Cl | 76-78 | |
| 205 | 4-CN, 2,6-di-F—Ph | 4-Cl—Ph | Cl | Me | | 429 |
| 206 | 2-Cl, 3,6-di-F—Ph | 4-F—Ph | Cl | Me | | 429 |
| 207 | 2-Cl, 3,6-di-F—Ph | 4-Me—Ph | Cl | Me | | 429 |
| 208 | 2-Cl, 3,6-di-F—Ph | 4-Cl, 3-F—Ph | Cl | H | | 429 |
| 209 | 2,6-di-F—Ph | 4-Cl, 3-F—Ph | Cl | H | | 429 |
| 210 | 2,6-di-F—Ph | 3-Cl—Ph | Cl | H | | 429 |
| 211 | 2,3,6-tri-F—Ph | 3,4-di-Me—Ph | Cl | H | | 429 |
| 212 | 4-Cl—Ph | 2,4,6-tri-F—Ph | Br | H | 137-140 | |
| 213 | 2,6-di-F—Ph | 3-Cl, 4-MeO—Bn | Cl | Cl | | 405 |
| 214 | 4-Cl—Ph | 2,6-di-F, 4-MeO—Ph | Br | Cl | 119-122 | |
| 215 | 2,6-di-F, 3-MeS—Ph | 4-Cl—Ph | Cl | H | | 371 |
| 216 | 4-Cl—Ph | 2,6-di-F—Ph | Me | Br | 156-158 | |
| 217 | 2,6-di-F—Ph | 4-Cl—Ph | H₂C=CH | Me | 141-143 | |
| 218 | 4-Cl—Ph | 2,4,6-tri-F—Ph | Br | Br | 130-131 | |
| 219 | 2-Cl, 3,6-di-F—Ph | 4-Cl, 3-F—Ph | Cl | Br | | 457 |
| 220 | 2-Cl, 3,6-di-F—Ph | 4-Cl, 3-F—Ph | Cl | Cl | | 413 |
| 221 | 2,6-di-F—Ph | 3-Cl—Ph | Cl | Br | | 405 |
| 222 | 2,6-di-F—Ph | 3-Cl—Ph | Cl | Cl | | 361 |
| 223 | 2,6-di-F—Ph | 4-Cl, 3-F—Ph | Cl | Br | | 423 |
| 224 | 2,3,6-tri-F—Ph | 3,4-di-Me—Ph | Cl | Br | | 417 |
| 225 | 2,6-di-F—Ph | 4-Cl, 3-F—Ph | Cl | Cl | | 379 |
| 226 | 2,3,6-tri-F—Ph | 3,4-di-Me—Ph | Cl | Cl | | 371 |
| 227 | 2-Cl, 6-F—Ph | 4-Me—Ph | Cl | Cl | 142-143 | |
| 228 | 2-Cl, 6-F—Ph | 4-F—Ph | Cl | Cl | 102-103 | |
| 229 | 2-Cl, 4-F—Ph | 4-Cl, 3-F—Ph | Cl | Cl | 116-117 | |
| 230 | 2-Cl, 6-F—Ph | 4-Cl, 3-F—Ph | Cl | Cl | 66-67 | |
| 231 | 2,6-di-F, 3-MeS—Ph | 4-Cl—Ph | Cl | Br | | 451 |
| 232 | 3-ClCH₂S, 2,6-di-F—Ph | 4-Cl—Ph | Cl | H | | 405 |
| 233 | 4-Cl—Ph | 2,4,6-tri-F—Ph | Br | Cl | 111-112 | |
| 234 | 2,3,6-tri-F—Ph | 3,4-di-Cl—Ph | Cl | Cl | | 413 |
| 235 | 2,6-di-F, 3-MeS—Ph | 4-Cl—Ph | Cl | Me | | 385 |
| 236 | 2,6-di-F—Ph | 3-Cl—Ph | Cl | Me | | 339 |
| 237 | 2-Cl, 3,6-di-F—Ph | 4-Cl, 3-F—Ph | Cl | Me | | 393 |
| 238 | 2,3,6-tri-F—Ph | 3,4-di-Me—Ph | Cl | Me | | 351 |
| 239 | 2,6-di-F, 4-MeO—Ph | 4-Cl—Ph | Cl | Cl | | 391 |
| 240 | 2,6-di-F, 4-MeO—Ph | 4-Cl, 3-F—Ph | Cl | Cl | | 407 |
| 241 | 2,6-di-F, 4-MeO—Ph | 4-Cl, 3-F—Ph | Cl | I | | 499 |
| 242 | 4-Cl—Ph | 2,6-di-F, 4-MeO—Ph | Br | Br | 137-139 | |
| 243 | 2-Cl, 4-F—Ph | 4-Cl, 3-F—Ph | Cl | Me | 89-91 | |
| 244 | 2-Cl, 6-F—Ph | 4-Cl, 3-F—Ph | Cl | Me | 140-142 | |
| 245 | 2,6-di-F, 4-MeO—Ph | 4-Cl—Ph | Cl | I | | 481 |
| 246 (Ex. 11) | 2,6-di-F, 4-MeO—Ph | 3-F—Ph | Cl | H | | 339 |
| 247 | 2,6-di-F—Ph | 4-Cl, 3-F—Ph | Cl | Me | | 358 |
| 249 | 4-Cl—Ph | 2,6-di-F, 4-MeO—Ph | Me | Me | 105-108 | |
| 250 | 2,6-di-F, 4-MeO—Ph | 2-pyridinyl | Me | H | | 302 |
| 251 | 2-pyridinyl | 4-Cl, 3-F—Ph | Me | H | | 288 |
| 252 | 3-pyridinyl | 4-Cl, 3-F—Ph | Me | H | | 288 |
| 253 | 4-pyridinyl | 4-Cl, 3-F—Ph | Me | H | | 288 |
| 254 | 2,6-di-F, 4-MeO—Ph | 6-Cl-3-pyridinyl | Me | H | | 336 |
| 255 | 3,5-di-Cl-4-pyridinyl | 4-Cl, 3-F—Ph | Me | H | | 356 |
| 256 (Ex. 12) | 2,6-di-F, 4-MeO—Ph | 3-F—Ph | Cl | Br | | 418 |
| 257 | 2,6-di-F, 4-MeO—Ph | 3-F—Ph | Cl | Cl | | 373 |
| 258 | 2,6-di-F—Bn | 4-Cl—Ph | Cl | Me | | 353 |
| 259 | 2-F—Ph | 4-Cl, 3-F—Ph | ClCH₂ | Cl | | 373 |
| 260 | 2-F—Ph | 4-Cl, 3-F—Ph | Me | Cl | | 339 |
| 261 | 2,6-di-F, 4-MeO—Ph | 4-Cl, 3-F—Ph | Me | Cl | | 387 |
| 262 | 3,5-di-Cl-4-pyridinyl | 4-Cl, 3-F—Ph | Me | Cl | | 390 |
| 263 | 2,6-di-F, 4-MeO—Ph | 6-Cl-3-pyridinyl | Me | Cl | | 370 |
| 264 | 2-pyridinyl | 4-Cl, 3-F—Ph | Me | Cl | | 322 |
| 265 | 2,6-di-F, 4-MeO—Ph | 4-Cl—Bn | Me₂N | H | | 378 |
| 266 | 2,3,6-tri-F—Ph | 4-Cl—Bn | Cl | H | ** | ** |
| 267 | 2,3,6-tri-F—Ph | 4-Cl—Bn | Me₂N | H | ** | ** |
| 268 | 2,6-di-F, 4-MeO—Ph | 4-Cl—Bn | Cl | H | ** | ** |
| 269 | 2,6-di-F, 4-MeO—Ph | 4-Cl—Bn | Me₂N | H | ** | ** |
| 270 | 2,6-di-F, 3-MeO—Ph | 4-Cl—Bn | Cl | H | ** | ** |
| 271 | 2,6-di-F, 3-MeO—Ph | 4-Cl—Bn | Me₂N | H | ** | ** |
| 272 | 4-Cl—Ph | 3-Cl—Bn | Cl | H | ** | ** |
| 273 | 4-Cl—Ph | 3-Cl—Bn | Me₂N | H | ** | ** |
| 274 | 2,6-di-F, 4-MeO—Ph | 4-Me—Bn | Cl | H | ** | ** |
| 275 | 2,6-di-F, 4-MeO—Ph | 4-Me—Bn | Me₂N | H | ** | ** |
| 276 (Ex. 20) | 2,6-di-F, 4-MeO—Ph | 4-F—Ph | Cl | HC(=O) | ** | ** |
| 277 | 4-Cl—Ph | 2,6-di-F, 4-MeO—Ph | Cl | H | 147-148 | |
| 278 | 2,6-di-F, 3-MeO—Ph | 3-F—Ph | Cl | Br | | 419 |
| 279 | 2,6-di-F, 3-MeO—Ph | 3-F—Ph | Cl | Cl | | 373 |
| 280 (Ex. 13) | 2,6-di-F, 4-MeO—Ph | 3-F—Ph | Cl | Me | | 354 |
| 281 | 2,6-di-F, 3-MeO—Ph | 4-Me—Ph | Cl | Br | | 415 |
| 282 | 2-Cl, 4-F—Ph | 4-Me—Ph | Cl | Cl | 72-73 | |
| 283 | 2,6-di-F, 4-MeO—Ph | 4-Cl—Bn | Cl | Br | 139-140 | |
| 284 | 2,6-di-F, 4-MeO—Ph | 4-Cl—Bn | Cl | Cl | 123-124 | |
| 285 | 2,3,6-tri-F—Ph | 4-Cl—Bn | Cl | Cl | ** | ** |
| 286 | 2,3,6-tri-F—Ph | 4-Cl—Bn | Cl | Br | 87-88 | |
| 287 | 2,6-di-F, 4-MeO—Ph | 4-Cl—Bn | Cl | Me | 116-117 | |
| 288 | 2,6-di-F, 3-MeO—Ph | 4-Cl—Bn | Cl | Cl | ** | ** |
| 289 | 2,6-di-F, 3-MeO—Ph | 4-Cl—Bn | Cl | Br | | 449 |
| 290 | 4-Cl—Ph | 3-Cl—Bn | Cl | Br | 108-109 | |
| 291 | 2,6-di-F, 4-MeO—Ph | 4-Me—Bn | Cl | Cl | | 383 |
| 292 | 2,6-di-F, 4-MeO—Ph | 4-Me—Bn | Cl | Br | 104-105 | |
| 293 | 2,3,6-tri-F—Ph | 4-Cl—Bn | Cl | Me | | 371 |
| 294 | 2,6-di-F, 3-MeO—Ph | 4-Cl—Bn | Cl | Me | | 384 |
| 295 | 4-Cl—Ph | 3-Cl—Bn | Cl | Me | 113-114 | |
| 296 | 2,6-di-F, 4-MeO—Ph | 4-Me—Bn | Cl | Me | | 363 |
| 297 | 4-Cl—Ph | 2,6-di-F, 4-MeO—Ph | Cl | Cl | 117-118 | |
| 298 (Ex. 21) | 2,6-di-F, 4-MeO—Ph | 4-F—Ph | Cl | HOCH₂ | ** | ** |
| 299 | 2,6-di-F, 3-MeO—Ph | 4-F—Ph | Cl | H | | 340 |
| 300 | 2,6-di-F, 3-MeO—Ph | 3-Me—Ph | Cl | H | | 336 |
| 301 | 4-Cl, 3-F—Ph | 2-Cl, 4-F—Ph | Br | Br | 104-106 | |
| 302 | 4-Cl, 3-F—Ph | 2-Cl, 4-F—Ph | Cl | Cl | 120-122 | |
| 303 | 2,6-di-F—Ph | 2-naphthalenyl | Cl | Me | 165-167 | |
| 304 | 2,6-di-F—Ph | 4-Cl-1-naphthalenyl | Cl | Me | 172-175 | |
| 305 | 2,6-di-F—Ph | 4-Cl—Ph | Et | Me | 122-124 | |
| 306 | 4-Cl—Ph | 2-Cl, 4-F—Ph | Br | Br | 114-116 | |
| 307 | 4-Cl—Ph | 2-Cl, 4-F—Ph | Me | Me | | 336 |
| 308 | 4-Cl—Ph | 2,4,6-tri-F—Ph | Me | Me | 90-92 | |
| 309 | 2,6-di-F, 3-MeO—Ph | 4-Cl—Ph | Cl | Cl | | 391 |
| 310 | 2,6-di-F, 3-MeO—Ph | 4-Me—Ph | Cl | Cl | | 369 |
| 314 | 2,6-di-F, 3-MeO—Ph | 4-F—Ph | Cl | Br | | 419 |
| 315 | 2,6-di-F, 3-MeO—Ph | 3-F—Ph | Cl | Me | | 353 |
| 316 | 2,6-di-F, 3-MeO—Ph | 3-Me—Ph | Cl | Br | | 414 |
| 317 | 2,6-di-F, 3-MeO—Ph | 4-Cl, 3-F—Ph | Cl | Br | | 453 |
| 318 | 2-F—Ph | 4-Cl, 3-F—Ph | Me | H | | 305 |
| 319 | 2-F-4-pyridinyl | 4-Cl, 3-F—Ph | Me | H | | 306 |
| 320 | 2,6-di-F—Ph | 5-Cl-2-pyridinyl | Me | H | ** | ** |
| 321 | 2,6-di-F—Ph | 5-Cl-2-pyridinyl | Me | Cl | ** | ** |
| 322 | 2-F-4-pyridinyl | 4-Cl, 3-F—Ph | ClCH₂ | Cl | ** | ** |
| 323 | 2-F-4-pyridinyl | 4-Cl, 3-F—Ph | Me | Cl | ** | ** |
| 324 | 4-Cl—Ph | 2-Cl, 4-F—Ph | Br | Cl | 106-109 | |
| 325 | 4-Cl, 3-F—Ph | 2-Cl, 4-F—Ph | Me | Br | 105-107 | |
| 326 | 4-Cl, 3-F—Ph | 2-Cl, 4-F—Ph | Br | Cl | 116-118 | |
| 327 | 2,6-di-F—Ph | 6-quinolinyl | Cl | Br | 176-180 | |
| 328 | 4-Cl, 3-F—Ph | 2-Cl, 4-F—Ph | Cl | Br | 120-122 | |
| 329 | 4-Cl—Ph | 2-Cl, 4-F—Ph | Cl | Br | 113-116 | |
| 330 | 2,3,6-tri-F—Ph | 4-Cl—Ph | Br | H | ** | ** |
| 331 (Ex. 22) | 2,6-di-F, 4-MeO—Ph | 4-F—Ph | Cl | FCH₂ | ** | ** |
| 332 | 2,3,6-tri-F—Ph | 4-F—Ph | Br | H | ** | ** |
| 333 | 2,6-di-F, 3-MeO—Ph | 4-Me—Ph | Cl | Me | | 349 |
| 334 | 2,6-di-F, 3-MeO—Ph | 4-F—Ph | Cl | Me | | 353 |
| 335 | 2,6-di-F, 3-MeO—Ph | 3-Me—Ph | Cl | Me | 113-117 | |
| 336 | 2,6-di-F—Ph | 2-naphthalenyl | Br | Me | 173-175 | |
| 337 | 2,6-di-F—Ph | 4-Cl-1-naphthalenyl | Br | Me | 192-195 | |
| 338 | 4-Cl—Ph | 2-Cl, 4-F—Ph | Br | Me | 127-131 | |
| 339 | 4-Cl—Ph | 2-Cl, 4-F—Ph | Me | Br | 146-150 | |
| 340 | 4-Cl, 3-F—Ph | 2-Cl, 4-F—Ph | Br | Me | 125-127 | |
| 341 | 4-Cl—Ph | 2,6-di-F—Ph | H | Cl | 77-79 | |
| 342 | 2,3,6-tri-F—Ph | 4-Cl, 3-F—Ph | Br | H | ** | ** |
| 343 | 2-Cl, 3,6-di-F—Ph | 4-Cl—Ph | Br | H | ** | ** |
| 345 | 2-Br-4-pyridinyl | 4-Cl, 3-F—Ph | Me | H | | 366 |
| 346 | 5-Br-3-pyridinyl | 4-Cl, 3-F—Ph | Me | H | | 366 |
| 347 | 5-Br-2-pyridinyl | 4-Cl, 3-F—Ph | Me | H | ** | ** |
| 348 | 2-Cl-3-pyridinyl | 4-Cl, 3-F—Ph | Me | H | | 322 |
| 349 (Ex. 23) | 2,6-di-F, 4-MeO—Ph | 4-F—Ph | Cl | HON═CH | ** | ** |
| 350 | 2,3,6-tri-F—Ph | 4-Cl, 3-F—Ph | Br | Cl | ** | ** |
| 351 | 2,3,6-tri-F—Ph | 4-Cl—Ph | Br | Cl | ** | ** |
| 352 | 2,3,6-tri-F—Ph | 4-F—Ph | Br | Cl | ** | ** |
| 353 | 2-Br-4-pyridinyl | 4-Cl, 3-F—Ph | ClCH₂ | Cl | | 434 |
| 354 | 2-Br-4-pyridinyl | 4-Cl, 3-F—Ph | Me | Cl | | 400 |
| 355 | 5-Br-3-pyridinyl | 4-Cl, 3-F—Ph | ClCH₂ | Cl | | 434 |
| 356 | 5-Br-3-pyridinyl | 4-Cl, 3-F—Ph | Me | Cl | | 400 |
| 357 | 5-Br-2-pyridinyl | 4-Cl, 3-F—Ph | ClCH₂ | Cl | ** | ** |
| 358 | 5-Br-2-pyridinyl | 4-Cl, 3-F—Ph | Me | Cl | | 400 |
| 359 | 2-Cl-3-pyridinyl | 4-Cl, 3-F—Ph | ClCH₂ | Cl | | 390 |
| 360 | 2-Cl-3-pyridinyl | 4-Cl, 3-F—Ph | Me | Cl | | 356 |
| 361 | 4-Cl—Ph | 3-Cl—Bn | Cl | Cl | 107-109 | |
| 362 | 2,4,6-tri-F—Ph | 3-F—Ph | Cl | H | 133-135 | |
| 363 | 2,4,6-tri-F—Ph | 3-F—Ph | Me₂N | H | ** | ** |
| 364 | 2,3,6-tri-F—Ph | 3-F—Ph | Cl | H | 78-81 | |
| 365 | 2,4,6-tri-F—Ph | 3-F—Ph | Cl | Cl | 78-81 | |
| 366 | 2,3,6-tri-F—Ph | 3-F—Ph | Cl | Cl | 143-145 | |
| 367 | 2,4,6-tri-F—Ph | 3-F—Ph | Cl | Br | 130-132 | |
| 368 | 2,3,6-tri-F—Ph | 3-F—Ph | Cl | Br | 137-138 | |
| 369 | 2,3,6-tri-F—Ph | 3-F—Ph | Cl | Me | 146-147 | |
| 370 | 2,4,6-tri-F—Ph | 3-F—Ph | Cl | Me | 150-152 | |
| 371 | 4,5-di-Br, 1-Me-1H- | 4-Cl—Ph | Br | Br | | 496 |
| | imidazol-2-yl | | | | | |
| 372 | 4,5-di-Br, 1-Me-1H- | 4-Cl—Ph | Br | Br | | 575 |
| | imidazol-2-yl | | | | | |
| 373 | 4-Cl—Ph | 2,6-di-F, 4-MeO—Ph | Cl | Br | 118-125 | |
| 374 | 2-Cl, 3,6-di-F—Ph | 4-Cl—Ph | Br | Br | ** | ** |
| 375 | 6-Me-2-pyridinyl | 4-Cl, 3-F—Ph | Me | H | ** | ** |
| 376 | 4-Cl-5-thiazolyl | 4-Cl, 3-F—Ph | Me | H | ** | ** |
| 377 | 2,4-di-Cl-5-thiazolyl | 4-Cl, 3-F—Ph | Me | H | ** | ** |
| 378 | 6-Me-2-pyridinyl | 4-Cl, 3-F—Ph | Me | Cl | ** | ** |
| 379 | 6-Me-2-pyridinyl | 4-Cl, 3-F—Ph | ClCH₂ | Cl | ** | ** |
| 380 | 4-Cl-5-thiazolyl | 4-Cl, 3-F—Ph | Me | Cl | ** | ** |
| 381 | 2,4-di-Cl-5-thiazolyl | 4-Cl, 3-F—Ph | Me | Cl | ** | ** |
| 382 | 2,3,6-tri-F—Ph | 4-Cl, 3-F—Ph | Br | Br | ** | ** |
| 383 | 2,3,6-tri-F—Ph | 4-Cl—Ph | Br | Br | | 467 |
| 384 | 2,3,6-tri-F—Ph | 4-F—Ph | Br | Br | | 449 |
| 385 | 2-Cl, 3,6-di-F—Ph | 4-Cl—Ph | Br | Cl | | 439 |
| 386 (Ex. 29) | 3-CN, 2,6-di-F—Ph | 4-Cl—Ph | Cl | H | | 350 |
| 387 | 3-CN, 2,6-di-F—Ph | 4-CN—Ph | Cl | H | | 341 |
| 388 | 2-Cl, 3,6-di-F—Ph | 4-Cl—Ph | Me | Me | | 353 |
| 389 | 2,3,6-tri-F—Ph | 4-Cl, 3-F—Ph | Me | Cl | ** | ** |
| 390 | 2,6-di-F, 3-MeO—Ph | 4-F—Ph | Cl | Cl | | 374 |
| 391 | 2,6-di-F, 3-MeO—Ph | 3-Me—Ph | Cl | Cl | 133-136 | |
| 392 | 2,6-di-F, 3-MeO—Ph | 4-Cl, 3-F—Ph | Cl | Cl | 84-86 | |
| 393 | 2,6-di-F, 3-MeO—Ph | 4-Cl, 3-F—Ph | Cl | Me | | 387 |
| 394 | 5-Br, 4-Cl, 1-Me-1H- | 4-Cl—Ph | H | Cl | | 407 |
| | imidazol-2-yl | | | | | |
| 396 | 4-Cl—Ph | 2,6-di-F, 4-MeO—Ph | Me | Cl | 104-106 | |
| 397 | 4-Cl—Ph | 2,6-di-F, 4-MeO—Ph | ClCH₂ | Cl | 127-129 | |
| 398 | 2,6-di-F, 3-MeO—Ph | 3-Et—Ph | Cl | Br | | 429 |
| 399 | 2,6-di-F, 3-MeO—Ph | 4-Cl, 3-Me—Ph | Cl | Cl | | 403 |
| 400 | 2,6-di-F, 3-MeO—Ph | 3-F₂CHO—Ph | Cl | Cl | 100-102 | |
| 401 | 2,6-di-F, 3-MeO—Ph | 4-Cl, 3-Me—Ph | Cl | Br | | 449 |
| 402 | 2,6-di-F, 3-MeO—Ph | 3-F₂CHO—Ph | Cl | Br | 109-111 | |
| 403 | 2,4-di-F-Bn | 4-Cl—Ph | H | Me | | 320 |
| 404 | 2,4-di-F-Bn | 4-Cl—Ph | Br | Me | | 399 |
| 405 | 3-Cl, 2,6-di-F, | 4-Me—Ph | Cl | Cl | ** | ** |
| | 4-MeO—Ph | | | | | |
| 406 | 2,6-di-F, 4-MeO—Ph | 6-Cl-3-pyridinyl | Cl | Cl | | 390 |
| 407 | 3-CN, 2,6-di-F—Ph | 4-Cl—Ph | Cl | Cl | | 386 |
| 408 | 4-Br, 5-MeS-2-thienyl | 4-Cl—Ph | Br | Br | | 545 |
| 409 | 2-Cl, 3,6-di-F—Ph | 4-Cl, 3-F—Ph | Br | H | ** | ** |
| 410 | 2-Cl, 3,6-di-F—Ph | 4-Me—Ph | Br | H | ** | ** |
| 411 | 2,3,6-tri-F—Ph | 4-Me—Ph | Br | H | ** | ** |
| 412 | 2,3,6-tri-F—Ph | 4-Cl—Ph | Me | Cl | ** | ** |
| 413 | 2,3,6-tri-F—Ph | 4-F—Ph | Me | Cl | ** | ** |
| 414 | 2-Cl, 3,6-di-F—Ph | 4-Cl—Ph | Me | Cl | ** | ** |
| 415 | 2,3,6-tri-F—Ph | 4-Cl, 3-F—Ph | Me | Me | ** | ** |
| 416 | 2,3,6-tri-F—Ph | 4-Cl—Ph | Me | Me | ** | ** |
| 417 | 2,3,6-tri-F—Ph | 4-F—Ph | Me | Me | ** | ** |
| 418 | 2,6-di-F, 3-MeO—Ph | 3-Et—Ph | Cl | Me | | 364 |
| 419 (Ex. 30) | 3-CN, 2,6-di-F—Ph | 4-Cl—Ph | Cl | Br | | 430 |
| 420 | 2,6-di-F, 4-MeO—Ph | Ph | Cl | H | | 321 |
| 421 | 4-Cl, 3-F—Ph | 2-Cl, 4-F—Ph | Cl | Me | 108-110 | |
| 422 | 4-Cl—Ph | 2-Cl, 4-F—Ph | Cl | Me | 135-137 | |
| 423 | 4-Cl—Ph | 2,6-di-F, 4-MeO—Ph | Cl | Me | 108-110 | |
| 425 | 2,6-di-F, 3-MeO—Ph | 4-Cl, 3-Me—Ph | Cl | Me | | 383 |
| 426 | 2,6-di-F, 3-MeO—Ph | 3-F₂CHO—Ph | Cl | Me | | 401 |
| 427 | 2,6-di-F, 3-MeO—Ph | 3-Et—Ph | Cl | Cl | 93-95 | |
| 428 | 3-Cl, 2,6-di-F, | Ph | Cl | Cl | | 391 |
| | 4-MeO—Ph | | | | | |
| 429 | 3-Br, 2,6-di-F, | Ph | Cl | Br | | 479 |
| | 4-MeO—Ph | | | | | |
| 430 (Ex. 31) | 3-CN, 2,6-di-F—Ph | 4-Cl—Ph | Cl | Me | | 364 |
| 433 | 6-Br-2-pyridinyl | 4-Cl, 3-F—Ph | Cl | Cl | | 420 |
| 434 | 6-MeO-3-pyridinyl | 4-Cl, 3-F—Ph | Cl | Cl | ** | ** |
| 435 | 3-F, 5-MeO-4-pyridinyl | 4-Cl, 3-F—Ph | Cl | Cl | | 390 |
| 436 | 3-quinolinyl | 4-Cl, 3-F—Ph | Cl | Cl | | 392 |
| 437 | 2,6-di-Cl, 4-MeO—Ph | 4-Cl, 3-F—Ph | Cl | Cl | ** | ** |
| 438 | 2,4-di-Cl, 6-MeO—Ph | 4-Cl, 3-F—Ph | Cl | Cl | ** | ** |
| 439 | 2,6-di-F, 4-MeO, 3- | Ph | Cl | Me | | 349 |
| | Me—Ph | | | | | |
| 440 | 2,3,6-tri-F—Ph | 3,4-di-Cl—Ph | Cl | Br | | 457 |
| 441 | 2,6-di-F, 3-MeO—Ph | 4-Et—Ph | Cl | Br | | 429 |
| 442 | 2,6-di-F, 3-MeO—Ph | 4-Et—Ph | Cl | Cl | | 383 |
| 443 | 2,6-di-F, 3-MeO—Ph | 4-I—Ph | Cl | Br | 134-138 | |
| 444 | 2,6-di-F, 3-MeO—Ph | 4-I—Ph | Cl | Cl | | 481 |
| 445 | 4-Cl—Ph | 2,4,6-tri-F—Ph | Me | Br | 123-125 | |
| 446 | 4-Cl—Ph | 2,4,6-tri-F—Ph | Me | Cl | 86-88 | |
| 447 | 4-Cl—Ph | 2,4,6-tri-F—Ph | Me | H | 110-112 | |
| 448 | 2,6-di-F, 3-MeO—Ph | 4-I—Ph | Cl | H | | 447 |
| 449 | 2,6-di-F, 3-MeO—Ph | 4-Cl—Ph | Br | H | | 401 |
| 450 (Ex. 14) | 2,6-di-F, 3-MeO—Ph | 4-F—Ph | Br | H | ** | ** |
| 451 | 3-Cl, 5-ClCH₂, 4-Me- | 4-Cl—Ph | H | Cl | | 393 |
| | 2-thienyl | | | | | |
| 452 | 5-ClCH₂, 4-Me-2- | 4-Cl—Ph | H | Cl | | 359 |
| | thienyl | | | | | |
| 453 | 2,6-di-F, 4-MeO—Ph | 3-Cl—Ph | Cl | Cl | ** | ** |
| 454 | 2,6-di-F, 4-MeO—Ph | 3,4-di-F—Ph | Cl | Cl | | 391 |
| 455 | 3-F-4-pyridinyl | 4-Cl, 3-F—Ph | Cl | Cl | | 360 |
| 456 | 4-Cl, 2-MeO-5- | 4-Cl, 3-F—Ph | Cl | Cl | | 412 |
| | thiazolyl | | | | | |
| 457 | 4-Cl-5-thiazolyl | 4-Cl, 3-F—Ph | Cl | Cl | | 482 |
| 458 | 2-Cl-3-pyridinyl | 4-Cl, 3-F—Ph | Cl | Cl | | 376 |
| 459 | 2-Cl, 3,6-di-F—Ph | 4-Cl, 3-F—Ph | Br | Br | ** | ** |
| 460 | 2-Cl, 3,6-di-F—Ph | 4-Me—Ph | Br | Br | ** | ** |
| 461 | 2,3,6-tri-F—Ph | 4-Me—Ph | Br | Br | ** | ** |
| 462 | 2,4,6-tri-F—Ph | 4-F—Ph | Br | Br | ** | ** |
| 463 | 2-Cl, 3,6-di-F—Ph | 4-Cl, 3-F—Ph | Br | Cl | ** | ** |
| 464 | 2-Cl, 3,6-di-F—Ph | 4-Me—Ph | Br | Cl | ** | ** |
| 465 | 2,3,6-tri-F—Ph | 4-Me—Ph | Br | Cl | ** | ** |
| 466 | 4-Cl—Ph | 2,6-di-F—Ph | Me | Cl | 127-129 | |
| 467 | 4-Cl—Ph | 2,6-di-F—Ph | MeOCH₂ | Cl | 95-97 | |
| 468 | 2,6-di-F—Ph | 6-quinolinyl | Cl | Me | 210-215 | |
| 469 | 2,6-di-F—Ph | 2,3-dihydro-1,4- | Cl | ClCH₂ | 142-144 | |
| | | benzodioxin-6-yl | | | | |
| 470 | 2,6-di-F—Ph | 2,3-dihydro-1,4- | Cl | Me | 122-124 | |
| | | benzodioxin-6-yl | | | | |
| 471 | 2,6-di-F, 3-MeO—Ph | 4-Cl—Ph | Br | Br | ** | ** |
| 472 | 2,6-di-F, 3-MeO—Ph | 4-F—Ph | Br | Cl | | 417 |
| 473 | 2,6-di-F, 3-MeO—Ph | 4-Me—Ph | Br | H | | 380 |
| 475 (Ex. 24) | 2,6-di-F—Ph | 4-Cl—Ph | Br | BrCH₂ | | 463 |
| 476 | 2,4-di-F—Bn | 3,5-di-MeO—Ph | H | Me | | 345 |
| 477 | 2,4-di-F—Bn | 2,6-di-Br, 3,5-di-MeO—Ph | Br | Me | | 582 |
| 479 | 2,6-di-F, 3-MeO—Ph | 4-Et—Ph | Cl | Me | | 364 |
| 480 | 2,3,6-tri-F—Ph | 3,4-di-Cl—Ph | Cl | Me | | 391 |
| 481 | 2-Cl, 6-F, 3-MeO—Ph | 4-Cl—Ph | Cl | Cl | | 407 |
| 482 | 2-Cl, 6-F, 5-MeO—Ph | 4-Cl—Ph | Cl | H | | 373 |
| 483 | 2,6-di-F, 4-MeO—Ph | 3-Cl—Ph | Cl | Br | | 435 |
| 484 | 2,6-di-F, 4-MeO—Ph | 3,4-di-F—Ph | Cl | Br | | 437 |
| 485 | 2-Br-4-pyridinyl | 4-Cl, 3-F—Ph | Cl | Cl | | 420 |
| 486 | 5-Br-3-pyridinyl | 4-Cl, 3-F—Ph | Cl | Cl | | 420 |
| 487 | 5-Br-2-pyridinyl | 4-Cl, 3-F—Ph | Cl | Cl | ** | ** |
| 488 | 2,6-di-F, 4-MeO—Ph | 6-CF₃-3-pyridinyl | Cl | Cl | ** | ** |
| 489 | 2-Cl, 3,6-di-F—Ph | 4-Cl, 3-F—Ph | Me | Me | ** | ** |
| 490 | 2,3,6-tri-F—Ph | 4-Me—Ph | Me | Me | ** | ** |
| 491 | 2,4,6-tri-F—Ph | 4-F—Ph | Me | Me | ** | ** |
| 492 | 2,4,6-tri-F—Ph | 4-F—Ph | Me | Cl | ** | ** |
| 493 | 2-Cl, 3,6-di-F—Ph | 4-Me—Ph | Me | Me | ** | ** |
| 494 | 2-Cl, 3,6-di-F—Ph | 4-Cl, 3-F—Ph | Me | Cl | ** | ** |
| 495 | 2-Cl, 3,6-di-F—Ph | 4-Me—Ph | Me | Cl | ** | ** |
| 496 | 2,3,6-tri-F—Ph | 4-Me—Ph | Me | Cl | ** | ** |
| 497 | 2,6-di-F, 4-MeO—Ph | 3-Cl—Ph | Cl | H | 114-115 | |
| 498 | 2,6-di-F, 4-MeO—Ph | 3,4-di-F—Ph | Cl | H | 115-116 | |
| 499 | 2,6-di-F, 4-MeO—Ph | 3,4-di-F—Ph | Cl | Me | 169-170 | |
| 500 | 2,6-di-F, 4-MeO—Ph | 3-Cl—Ph | Cl | Me | 173-174 | |
| 501 | 2-Cl, 6-F, 3-MeO—Ph | 4-Cl—Ph | Cl | Br | 154-156 | |
| 502 | 2,6-di-F, 4-MeO—Ph | 3-Cl, 4-Me—Ph | Cl | Cl | 143-145 | |
| 503 | 4-Cl—Ph | 2-Cl, 4-F—Ph | Me | Cl | 123-127 | |
| 504 (Ex. 15) | 2,6-di-F, 3-MeO—Ph | 4-F—Ph | Br | Br | 134-136 | |
| 505 | 2,6-di-F, 4-MeO—Ph | 4-Me—Ph | Br | H | | 385 |
| 506 | 2,6-di-F, 4-MeO—Ph | 4-F—Ph | Br | H | ** | ** |
| 507 | 2,4,6-tri-F—Ph | 4-F—Ph | Br | H | ** | ** |
| 508 | 2-Cl, 3,6-di-F—Ph | 4-F—Ph | Br | H | ** | ** |
| 509 (Ex. 25) | 2,6-di-F—Ph | 4-Cl—Ph | Br | N≡CCH₂ | | 410 |
| 510 | 2,6-di-F, 3-MeO—Ph | 4-Cl—Ph | Br | Cl | ** | ** |
| 511 | 2,6-di-F, 3-MeO—Ph | 4-Me—Ph | Br | Br | | 459 |
| 512 | 2-Cl, 6-F, 5-MeO—Ph | 4-Cl—Ph | Cl | Br | | 451 |
| 513 | 2,6-di-F, 4-MeO—Ph | 2-Cl-3-pyridinyl | Cl | Cl | | 390 |
| 514 | 2,6-di-F, 4-MeO—Ph | 6-Cl, 2-MeO-3-pyridinyl | Cl | Cl | ** | ** |
| 515 | 2,6-di-F, 4-MeO—Ph | 2-Cl, 6-MeO-3-pyridinyl | Cl | Cl | | 420 |
| 516 | 2,6-di-F, 4-MeO—Ph | 6-MeO-3-pyridinyl | Cl | Cl | | 386 |
| 517 | 2,6-di-F, 3-MeO—Ph | 4-Me—Ph | Br | Cl | | 415 |
| 518 | 2-Cl, 6-F, 5-MeO—Ph | 4-Cl—Ph | Cl | Cl | | 407 |
| 519 | 2-Cl, 6-F, 3-MeO—Ph | 4-Cl—Ph | Cl | Me | | 387 |
| 520 | 2,6-di-F, 3-MeO—Ph | 3-F, 4-Me—Ph | Cl | H | | 354 |
| 521 | 2,6-di-F, 4-MeO—Ph | 4-Me—Ph | Me | Me | ** | ** |
| 522 | 2,6-di-F, 4-MeO—Ph | 4-F—Ph | Me | Me | ** | ** |
| 523 | 2,6-di-F, 4-MeO—Ph | 4-F—Ph | Br | Br | ** | ** |
| 524 | 2,6-di-F, 4-MeO—Ph | 4-Me—Ph | Br | Br | ** | ** |
| 525 | 4-Me—Ph | 2,6-di-F, 4-MeO—Ph | Me | Br | 128-129 | |
| 526 | 2,6-di-F, 3-MeO—Ph | 4-Cl—Ph | Me | Cl | | 369 |
| 527 | 2,6-di-F, 3-MeO—Ph | 4-CN—Ph | Cl | H | | 346 |
| 528 | 2,6-di-F, 3-MeO—Ph | 4-Br—Ph | Cl | H | | 401 |
| 529 | 2,6-di-F—Ph | 4-ClCH₂S—Ph | Cl | Me | | 386 |
| 530 | 2,4,6-tri-F—Bn | 4-Cl—Ph | H | Me | | 338 |
| 531 | 2,4,6-tri-F—Bn | 4-Cl—Ph | Cl | Me | | 372 |
| 532 | 2,4,6-tri-F—Bn | 4-Cl—Ph | Br | Me | | 417 |
| 533 | 2,4,6-tri-F—Bn | 3,5-di-MeO—Ph | H | Me | | 363 |
| 534 | 2,4,6-tri-F—Bn | 3,5-di-MeO—Ph | Cl | Me | | 398 |
| 535 | 2,4,6-tri-F—Bn | 3,5-di-MeO—Ph | Br | Me | | 442 |
| 536 | 2,4,6-tri-F—Bn | 2,6-di-Cl—Ph | H | Me | | 372 |
| 537 | 2,4,6-tri-F—Bn | 2,6-di-Cl—Ph | Cl | Me | | 407 |
| 538 | 2,4,6-tri-F—Bn | 2,6-di-Cl—Ph | Br | Me | | 451 |
| 539 | 2,4,6-tri-F—Bn | 3,5-di-Cl—Ph | H | Me | | 372 |
| 540 | 2,4,6-tri-F—Bn | 3,5-di-Cl—Ph | Cl | Me | | 407 |
| 541 | 2,4,6-tri-F—Bn | 3,5-di-Cl—Ph | Br | Me | | 451 |
| 542 | 2,6-di-F, 4-MeO—Ph | 3-Cl, 4-Me—Ph | Cl | Br | 161-163 | |
| 543 | 4-Cl, 3-F—Ph | 2-Cl, 4-F—Ph | Me | Me | | 354 |
| 544 (Ex. 26) | 2,6-di-F—Ph | 4-Cl—Ph | Br | MeOC(═O)CH₂ | | 443 |
| 545 | 2,6-di-F, 4-MeO—Ph | 3-F₂CHO—Ph | Cl | Br | | 467 |
| 546 | 2,6-di-F, 4-MeO—Ph | 4-F₂CHO—Ph | Cl | Br | | 467 |
| 547 | 2,6-di-F, 4-MeO—Ph | 3-CF₃O—Ph | Cl | Br | | 485 |
| 548 | 2,6-di-F, 4-MeO—Ph | 4-CF₃O—Ph | Cl | Br | 80-82 | |
| 549 (Ex. 16) | 2,6-di-F, 3-MeO—Ph | 4-F—Ph | Me | Me | | 333 |
| 550 | 2-Cl, 6-F, 5-MeO—Ph | 4-Cl—Ph | Cl | Me | | 387 |
| 551 | 2,6-di-F, 3-MeO—Ph | 3-F, 4-Me—Ph | Cl | Br | | 432 |
| 552 | 2,6-di-F, 3-MeO—Ph | 4-CN—Ph | Cl | Cl | 191-193 | |
| 553 | 2,6-di-F, 4-MeO—Ph | 6-Me-3-pyridinyl | Cl | Cl | | 370 |
| 554 | 2,6-di-F, 4-MeO—Ph | 6-Me-3-pyridinyl | Br | Br | | 458 |
| 555 | 2,6-di-F, 4-MeO—Ph | 6-Me-3-pyridinyl | Br | H | | 380 |
| 556 | 2,6-di-F, 4-MeO—Ph | 3-F₂CHO—Ph | Cl | H | 102-103 | |
| 557 | 2,6-di-F, 4-MeO—Ph | 4-F₂CHO—Ph | Cl | H | 74-76 | |
| 558 | 2,6-di-F, 4-MeO—Ph | 3-CF₃O—Ph | Cl | H | 85-86 | |
| 559 | 2,6-di-F, 4-MeO—Ph | 4-CF₃O—Ph | Cl | H | 115-116 | |
| 560 | 4-Me—Ph | 2,6-di-F, 4-MeO—Ph | Me | H | 114-118 | |
| 561 | 2,6-di-F, 3-MeO—Ph | 3-F, 4-Me—Ph | Cl | Cl | | 387 |
| 562 | 2,6-di-F, 3-MeO—Ph | 4-Br—Ph | Cl | Cl | | 435 |
| 563 | 2,6-di-F, 3-MeO—Ph | 4-Br—Ph | Cl | Br | | 479 |
| 564 | 4-Me—Ph | 2,6-di-F, 4-MeO—Ph | Me | Cl | 121-122 | |
| 565 | 2,6-di-F, 4-MeO—Ph | 3-F₂CHO—Ph | Cl | Cl | 68-70 | |
| 566 | 2,6-di-F, 4-MeO—Ph | 4-F₂CHO—Ph | Cl | Cl | | 421 |
| 567 | 2,6-di-F, 4-MeO—Ph | 3-CF₃O—Ph | Cl | Cl | 94-95 | |
| 568 | 2,6-di-F, 4-MeO—Ph | 4-CF₃O—Ph | Cl | Cl | 90-91 | |
| 569 | 2,6-di-F, 4-MeO—Ph | 4-F₂CHO—Ph | Cl | Me | | 401 |
| 570 (Ex. 27) | 2,6-di-F—Ph | 4-Cl—Ph | Br | HOC(═O)CH₂ | | 429 |
| 571 (Ex.28) | 2,6-di-F—Ph | 4-Cl—Ph | Br | MeNHC(═O)CH₂ | | 442 |
| 572 | 2,6-di-F, 4-MeO—Ph | 3-CN, 4-Me—Ph | Cl | Cl | 141-143 | |
| 573 | 4-Cl, 3-F—Ph | 2-Cl, 4-F—Ph | Me | Cl | 106-109 | |
| 574 | 2,6-di-Cl, 4-MeO—Ph | 4-Cl, 3-F—Ph | Cl | Br | ** | ** |
| 575 | 4-CN, 2-F, 6-MeO—Ph | 4-Cl, 3-F—Ph | Cl | Cl | ** | ** |
| 576 | 2,6-di-F, 4-MeO—Ph | 2-Br-3-pyridinyl | Cl | Cl | ** | ** |
| 577 | 2,6-di-F—Ph | 5-Me-2-pyridinyl | Cl | Cl | ** | ** |
| 581 | 4-Me—Ph | 2,6-di-F, 4-MeO—Ph | ClCH₂ | Cl | ** | ** |
| 582 | 2,6-di-F, 3-MeO—Ph | 4-Br—Ph | Cl | Me | | 415 |
| 583 (Ex. 17) | 2,6-di-F, 3-MeO—Ph | 3-F₂CHO—Ph | Cl | H₂C═CH | | 413 |
| 584 | 2,4,6-tri-F—Ph | 3-F₂CHO—Ph | Cl | H | | 375 |
| 585 | 2,4,6-tri-F—Ph | 4-F₂CHO—Ph | Cl | H | | 375 |
| 586 | 2,4,6-tri-F—Ph | 3-CF₃O—Ph | Cl | H | | 393 |
| 587 | 2,4,6-tri-F—Ph | 4-CF₃O—Ph | Cl | H | | 393 |
| 588 | 2,6-di-F, 4-MeO—Ph | 3-F₂CHO—Ph | Cl | Me | | 401 |
| 589 | 2,6-di-F, 4-MeO—Ph | 3-CF₃O—Ph | Cl | Me | | 419 |
| 590 | 2,6-di-F, 3-MeO—Ph | 3-F, 4-Me—Ph | Cl | Me | | 367 |
| 591 | 2,6-di-F—-Ph | Ph | Cl | Cl | | 325 |
| 592 | 2,6-di-F—-Ph | 6-Cl-3-pyridinyl | Cl | Cl | ** | ** |
| 593 | 3,5-di-F-4-pyridinyl | 4-Cl, 3-F—Ph | Cl | Cl | ** | ** |
| 594 | 3,5-di-F-4-pyridinyl | 4-Cl, 3-F—Ph | Cl | H | ** | ** |
| 596 | 2,6-di-F, 3-MeO—Ph | 4-CN-Ph | Cl | Br | | 426 |
| 597 | 2,4,6-tri-F—Ph | 3-F₂CHO—Ph | Cl | Cl | 82-83 | |
| 598 | 2,4,6-tri-F—Ph | 4-F₂CHO—Ph | Cl | Cl | 91-92 | |
| 599 | 2,4,6-tri-F—Ph | 3-CF₃O—Ph | Cl | Cl | 76-77 | |
| 600 | 2,4,6-tri-F—Ph | 4-CF₃O—Ph | Cl | Cl | 75-76 | |
| 601 | 2,6-di-F, 4-MeO—Ph | 3-Cl, 4-Me—Ph | Cl | Me | 143-145 | |
| 602 | 2,6-di-F—Ph | 2,3-dihydro-1,4- | Br | Me | 203-207 | |
| | | benzodioxin-6-yl | | | | |
| 603 | 2,4,6-tri-F—Ph | 3-CF₂HO—Ph | Cl | Br | 89-90 | |
| 604 | 2,4,6-tri-F—Ph | 4-F₂CHO—Ph | Cl | Br | 94-95 | |
| 605 | 2,4,6-tri-F—Ph | 3-CF₃O—Ph | Cl | Br | 80-81 | |
| 606 | 2,4,6-tri-F—Ph | 4-CF₃O—Ph | Cl | Br | 87-88 | |
| 607 | 2,6-di-F, 4-MeO—Ph | 3-CN, 4-Me—Ph | Cl | Br | 145-147 | |
| 608 | 2,6-di-F—Ph | 2,2-difluoro-1,3- | Cl | Me | 100-105 | |
| | | benzodioxol-5-yl | | | | |
| 609 | 2,4,6-tri-F—Ph | 3-F₂CHO—Ph | Cl | Me | | 389 |
| 610 | 2,4,6-tri-F—Ph | 4-F₂CHO Ph | Cl | Me | 96-97 | |
| 611 | 2,4,6-tri-F—Ph | 3-CF₃O—Ph | Cl | Me | 93-94 | |
| 612 | 2,4,6-tri-F—Ph | 4-CF₃O—Ph | Cl | Me | 81-82 | |
| 613 | 2,6-di-F, 4-MeO—Ph | 3-CN, 4-Me—Ph | Cl | Me | 123-125 | |
| 614 | 2,6-di-F—Ph | 4-MeOC(═O)—Ph | Cl | H | 132-133 | |
| 615 | 2,6-di-F, 3-MeO—Ph | 4-CN—Ph | Cl | Me | | 360 |
| 616 | 2,6-di-F—Ph | 2-MeO-3-pyridinyl | Cl | Cl | ** | ** |
| 617 | 2,6-di-F—Ph | 2-Cl-3-pyridinyl | Cl | Cl | | 360 |
| 618 | 2,6-di-F—Ph | 2-Cl-3-pyridinyl | Br | Br | | 448 |
| 619 | 2,6-di-F—Ph | 6-Cl, 2-MeO-3-pyridinyl | Cl | Cl | | 390 |
| 620 | 2,6-di-F—Ph | 2-Cl, 6-MeO-3-pyridinyl | Cl | Cl | | 390 |
| 621 | 2,6-di-F—Ph | 6-MeO-3-pyridinyl | Cl | Cl | | 356 |
| 622 | 2,6-di-F—Ph | 6-MeO-3-pyridinyl | Br | Br | | 444 |
| 623 | 3-Br, 2,6-di-F—Ph | 3-F—Ph | Cl | H | | 389 |
| 624 (Ex. 32) | 2,6-di-F, 4-OH—Ph | 4-Me—Ph | Cl | Me | | 335 |
| 631 | 2,6-di-F, 4-F₂CHO—Ph | 4-Me—Ph | Cl | Me | | 385 |
| 632 (Ex. 9) | 2,6-di-F, 4-MeO—Ph | 3,5-di-MeO—Ph | Me | H | *** | *** |
| 633 | 2,6-di-F—Ph | 4-CH₃OC(═O)—Ph | Me2N | H | 203-204 | |
| 634 | 2,6-di-F—Ph | 4-Me₂N—Ph | Me2N | H | 143-144 | |
| 635 | 2,6-di-F—Ph | 4-Me₂N—Ph | Cl | H | 164-165 | |
| 636 | 2,6-di-F—Ph | 4-CH₃C(═O)NH, 3-Cl—Ph | Cl | Cl | 171-172 | |
| 637 | 2,6-di-F—Ph | 4-CH₃C(═O)NH—Ph | Cl | Cl | 191-195 | |
| 638 | 2,6-di-F, 4-MeO—Ph | 4-Cl—Ph | Cl | NH₂ | 371 | |
| 641 | 2-F, 4-Cl—Ph | 2-Cl, 3,5-di-MeO—Ph | Br | H | | 447 |
| 642 | 2-F, 4-Cl—Ph | 2-Br, 6-Cl, 3,5-di-MeO—Ph | Br | H | | 525 |
| 643 | 2,6-di-F—Ph | 4-CH₃C(═O)NH—Ph | Cl | Br | 224-225 | |
| 645 | 2,6-di-F, 4-MeO—Ph | 3-Cl, 4-MeO—Ph | Cl | Br | 126-128 | |
| 646 | 2,6-di-F, 4-MeO—Ph | 3-Cl, 4-MeO—Ph | Cl | Cl | 113-115 | |
| 647 | 2,6-di-F, 4-MeO—Ph | 3-I, 4-Me—Ph | Cl | Cl | 105-107 | |
| 649 | 2,6-di-F—Ph | 4-MeOC(═O)—Ph | Cl | Cl | 173-174 | |
| 650 | 2,6-di-F—Ph | 3-Cl, 4-Me₂N—Ph | Cl | H | 181-182 | |
| 651 | 3,5-di-MeO—Ph | 2,6-di-F, 4-NO₂—Ph | Br | Me | | 456 |
| 652 | 2,6-di-F—Ph | 3,5-di-Cl, 4-Me₂N—Ph | Cl | H | 117-120 | |
| 653 | 2,6-di-F,4-MeO—Ph | 2-F, 4-Me—Ph | Cl | Cl | 120-122 | |
| 654 | 2,6-di-F, 4-MeO—Ph | 2-F, 4-Me—Ph | Cl | Br | 144-146 | |
| 655 | 2,6-di-F, 4-MeO—Ph | 3-I, 4-Me—Ph | Cl | Br | 127-129 | |
| 656 | 3-CN, 2,6-di-F—Ph | 4-F—Ph | Cl | H | | 334 |
| 657 | 2,6-di-F, 4-MeO—Ph | 3,5-di-MeO—Ph | Me | Br | | 441 |
| 658 | 2,6-di-F, 4-MeO—Ph | 2,6-di-Br, 3,5-di-MeO—Ph | Me | Br | | 599 |
| 659 | 2,6-di-F, 4-MeO—Ph | 2,6-di-Br, 3,5-di-MeO—Ph | Me | H | | 518 |
| 660 | 3-EtO, 2,6-di-F—Ph | 4-Cl—Ph | Cl | H | | 369 |
| 661 | 3-EtO, 2,6-di-F—Ph | 3-F—Ph | Cl | H | | 353 |
| 662 | 3-EtO, 2,6-di-F—Ph | 4-Cl—Ph | Cl | Br | | 449 |
| 663 | 3-EtO, 2,6-di-F—Ph | 3-F—Ph | Cl | Br | | 433 |
| 664 | 3-EtO, 2,6-di-F—Ph | 4-Cl—Ph | Cl | Cl | | 403 |
| 665 | 3-EtO, 2,6-di-F—Ph | 3-F—Ph | Cl | Cl | | 487 |
| 666 | 2,6-di-F—-Ph | 6-Cl-3-pyridinyl | Cl | Me | | 341 |
| 667 | 2,6-di-F, 4-MeO—Ph | 6-Cl-3-pyridinyl-CH₂ | Cl | Cl | | 404 |
| 668 | 3-Cl, 2,6-di-F, | Cl-3-pyridiny-CH₂ | Cl | Cl | | 440 |
| | 4-MeO—Ph | | | | | |
| 669 | 2,6-di-F, 4-MeO—Ph | 6-Cl-3-pyridinyl-CH₂ | Cl | H | 127-128 | |
| 670 | 2,6-di-F, 4-MeO—Ph | 6-Cl-3-pyridinyl-CH₂ | Cl | Br | | 450 |
| 671 | 2,6-di-F, 4-MeO—Ph | 6-Me-3-pyridinyl-CH₂ | Cl | Me | | 364 |
| 672 | 2,6-di-F, 4-MeO—Ph | 4-Cl—Ph | OH | Me | ** | ** |
| 673 | 2,6-di-F, 4-MeO—Ph | 6-CH₃C(═O)NH-3- | Cl | Cl | | 413 |
| | | pyridinyl | | | | |
| 674 | 2,6-di-F, 4-MeO—Ph | 2,6-di-Cl-3-pyridinyl | Cl | Cl | | 424 |
| 675 | 2,6-di-F, 4-MeO—Ph | 2-Me-3-pyridinyl | Cl | Cl | | 370 |
| 676 | 2,6-di-F, 4-MeO—Ph | 2-MeO-3-pyridinyl | Cl | Cl | | 386 |
| 677 | 2,6-di-F, 4-MeO—Ph | 2-Cl, 5-Me-3-pyridinyl | Cl | Cl | | 404 |
| 678 | 2,6-di-F, 4-MeO—Ph | 5-Br-3-pyridinyl | Cl | Cl | | 434 |
| 679 | 3-CN, 2,6-di-F—Ph | 4-F—Ph | Cl | Cl | | 368 |
| 680 | 3-CN, 2,6-di-F—Ph | 4-Me—Ph | Cl | Cl | | 364 |
| 681 | 3-CN, 2,6-di-F—Ph | 3-F—Ph | Cl | Cl | | 368 |
| 682 | 2,6-di-F—Ph | 6-Cl-3-pyridinyl | Me | Br | | 386 |
| 683 | 3-F—Ph | 2,6-di-F, 4-MeO—Ph | Me | H | 128-130 | |
| 684 | 3-EtO, 2,6-di-F—Ph | 4-Cl—Ph | Cl | Me | | 383 |
| 685 (Ex. 37) | 6-Cl-3-pyridinyl | 2,6-di-F, 4-MeO—Ph | Me | H | 117-118 | |
| 686 | 3-F—Ph | 2,6-di-F, 4-MeO—Ph | Me | Cl | ** | ** |
| 687 | 3-F—Ph | 2,6-di-F, 4-MeO—Ph | Me | Br | 113-114 | |
| 688 | 4-Cl—Ph | 2,6-di-F, 4-MeO—Ph | Me | H | 145-148 | |
| 689 | 2,6-di-F—Ph | 3-Br, 4-Me₂N—Ph | Me₂N | Br | 150-151 | |
| 690 | 2,6-di-F—Ph | 4-MeOC(═O)—Ph | Cl | Br | 186-187 | |
| 691 | 3-CN, 2,6-di-F—Ph | 6-Cl-3-pyridinyl | Cl | Cl | | 387 |
| 692 | 2,6-di-F, 4-MeO—Ph | 4-Cl—Ph | Cl | MeNH | 385 | |
| 693 | 2,6-di-F—Ph | 6-Cl-3-pyridinyl | Me | Cl | | 341 |
| 694 (Ex. 10) | 2,6-di-F, 4-MeO—Ph | 3,5-di-MeO—Ph | Me | Cl | *** | *** |
| 695 | 2,6-di-F, 4-MeO—Ph | 4-Me—Ph | Cl | MeS | | 381 |
| 696 (Ex. 38) | 6-Cl-3-pyridinyl | 2,6-di-F, 4-MeO—Ph | Me | Cl | 94-95 | |
| 697 | 3-CN, 2, 6-di-F—Ph | 6-Cl-3-pyridinyl | H | Cl | | 351 |
| 698 | 4-MeO—Ph | 2,4-di-Cl—Ph | Cl | Cl | | 389 |
| 699 | 2,6-di-F, 4-MeO—Ph | 6-CF3-3-pyridinyl | H | Cl | | 390 |
| 700 | 4-Cl—Ph | 2,6-di-F, 4-NO₂—Ph | H | Me | | 350 |
| 701 | 3-CN, 2,6-di-F—Ph | 6-Cl-3-pyridinyl | Cl | Br | | 429 |
| 702 | 4-EtO, 2,6-di-F—Ph | 4-F—Ph | Cl | H | | 353 |
| 703 | 4-EtO, 2,6-di-F—Ph | 3-F—Ph | Cl | H | | 353 |
| 705 | 2,6-di-F, 4-MeO—Ph | 6-MeO-3-pyridinyl | Cl | Br | | 432 |
| 706 | 2,6-di-F, 4-MeO—Ph | 6-CF3-3-pyridinyl | Br | Cl | | 470 |
| 707 (Ex. 35) | 2,6-di-F, 4-MeO—Ph | 6-CF3-3-pyridinyl | Cl | Br | | 470 |
| 708 | 4-Cl—Ph | 2,6-di-F, 4-NO₂—Ph | Cl | Me | | 384 |
| 709 | 2,6-di-F, 3-MeO—Ph | 6-Cl-3-pyridinyl | Cl | Cl | | 392 |
| 710 | 2,6-di-F, 4-MeO—Ph | 6-MeO-3-pyridinyl | Br | Cl | | 432 |
| 711 | 2,6-di-F—Ph | 6-Cl-3-pyridinyl | Cl | Br | | 406 |
| 712 | 3-F—Ph | 2,6-di-F, 4-MeO—Ph | ClCH₂ | Cl | | |
| 713 (Ex. 36) | 2,6-di-F, 4-MeO—Ph | 6-CF₃-3-pyridinyl | Cl | Me | | 404 |
| 714 | 2,6-di-F, 4-MeO—Ph | 6-MeO-3-pyridinyl | Cl | Me | | 366 |
| 715 | 3-CN, 2,6-di-F—Ph | 6-Cl-3-pyridinyl | Me | Br | | 409 |
| 716 | 3-CN, 2,6-di-F—Ph | 6-Cl-3-pyridinyl | Me | Cl | | 365 |
| 717 | 3-CN, 2,6-di-F—Ph | 6-Cl-3-pyridinyl | ClCH₂ | Cl | | 401 |
| 718 | 2,6-di-F—Ph | 6-MeO-3-pyridinyl | Me | Cl | | 337 |
| 719 | 2,6-di-F—Ph | 6-MeO-3-pyridinyl | Me | Br | | 381 |
| 723 | 4-EtO, 2,6-di-F—Ph | 4-Me—Ph | Cl | H | | 349 |
| 724 | 3-EtO, 2,6-di-F—Ph | 3-F—Ph | Cl | Me | | 367 |
| 725 | 2-Cl, 4-MeO—Ph | 4-F—Ph | Cl | Br | 115-118 | |
| 726 | 2-Cl, 4-MeO—Ph | 4-F—Ph | Cl | H | 112-114 | |
| 727 | 2,6-di-F, 4-MeO—Ph | 3,5-di-Cl-2-pyridinyl- | Cl | Cl | | 452 |
| | | CH(Me) | | | | |
| 728 | 2,6-di-F, 4-MeO—Ph | 5-F-3-pyridinyl | Cl | Cl | | 374 |
| 729 | 2,6-di-F, 4-MeO—Ph | 6-Cl-3-pyridinyl | Cl | Br | | 434 |
| 730 | 2,6-di-F, 3-MeO—Ph | 6-Cl-3-pyridinyl | Cl | Br | | 436 |
| 731 | 2,6-di-F, 4-MeO—Ph | 6-CF3-3-pyridinyl | Me | Br | | 450 |
| 732 | 2,6-di-F, 4-MeO—Ph | 6-CF3-3-pyridinyl | Me | Cl | | 404 |
| 733 | 2,6-di-F, 3-MeO—Ph | 6-Cl-3-pyridinyl | Me | Cl | | 370 |
| 734* | 2,6-di-F, 4- | 4-Me—Ph | Cl | Me | | 406 |
| (Ex. 33) | (MeNHCH₂CH₂CH₂)—Ph | | | | | |
| 735 | 2,6-di-F, 3-MeO—Ph | 6-Cl-3-pyridinyl | Me | Br | | 416 |
| 736 | 2,6-di-F, 4-MeO—Ph | 6-MeO-3-pyridinyl | Me | Br | | 412 |
| 737 | 2,6-di-F, 4-MeO—Ph | 6-MeO-3-pyridinyl | Me | Cl | | 366 |
| 738 | 2,6-di-F, 3-MeO—Ph | 6-Cl-3-pyridinyl | Cl | Me | | 370 |
| 739 | 2,6-di-F, 4-MeO—Ph | 6-Cl-3-pyridinyl | Cl | Me | | 370 |
| 740 (Ex. 6) | 2,6-di-F—Ph | 4-Cl—Ph | Br | Me | *** | *** |
| 741(Ex. 43) | 2,6-di-F, 4-MeO—Ph | 4-CNS—Ph | Cl | H | *** | *** |

*HCl salt.
**See Index Table D for 1H NMR data.
***See synthesis example for ¹H NMR data.

INDEX TABLE B

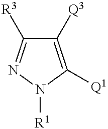

| Cmpd. | Q¹ | Q³ | R¹ | R³ | m.p. (° C.) | AP⁺ (M + 1) |
|---|---|---|---|---|---|---|
| 26 | Ph | 2,4-di-F—Ph | Me | Me | ** | ** |
| 31 | Ph | 2-Cl, 4-F—Ph | Me | Me | ** | ** |
| 60 (Ex. 40) | 2,6-di-F—Ph | 3-F—Ph | Me | Me | | 303 |
| 61 | 2-Cl, 6-F—Ph | 4-Cl—Ph | Me | Me | | 335 |
| 62 | 2-Cl, 6-F—Ph | 3-F—Ph | Me | Me | | 319 |
| 63 | 2-Cl, 6-F—Ph | 4-Cl, 3-F—Ph | Me | Me | | 353 |
| 67 | 4-Cl, 3-F—Ph | 2,6-di-F—Ph | Me | Me | | 337 |
| 68 | 2,6-di-F—Ph | 4-Cl, 3-F—Ph | Me | Me | | 337 |
| 69 | 2,4,6-tri-F—Ph | 4-Cl—Ph | Me | Me | | 337 |
| 70 | 2,4,6-tri-F—Ph | 3-F—Ph | Me | Me | | 321 |
| 71 | 2,4,6-tri-F—Ph | 4-Cl, 3-F—Ph | Me | Me | | 355 |
| 77 | 2,6-di-F, 4-MeO—Ph | 4-Cl—Ph | Me | Me | | 349 |
| 78 | 2,6-di-F, 4-MeO—Ph | 3-F—Ph | Me | Me | | 333 |
| 79 | 2,6-di-F, 4-MeO—Ph | 4-Cl, 3-F—Ph | Me | Me | | 367 |
| 87 | 2,6-di-Cl—Ph | 4-Cl—Ph | Me | Me | | 351 |
| 88 | 2,6-di-Cl—Ph | 3-F—Ph | Me | Me | | 335 |
| 89 | 2,6-di-Cl—Ph | 4-Cl, 3-F—Ph | Me | Me | | 369 |
| 104 | 4-Cl, 3-F—Ph | 2,4,6-tri-F—Ph | Me | Me | | 355 |
| 111 | 4-Cl, 3-MeO—Ph | 2,6-di-F, 4-MeO—Ph | Me | Me | | 379 |
| 115 | 4-Cl, 3-F—Ph | 2,6-di-F, 4-MeO—Ph | Me | Me | | 367 |
| 125 (Ex. 39) | 4-Cl—Ph | 2,6-di-F—Ph | Me | Br | | 385 |
| 165 | 4-Cl—Ph | 2,6-di-F—Ph | Et | Br | ** | ** |
| 166 | 4-Cl—Ph | 2,4,6-tri-F—Ph | Me | Br | | 403 |
| 311 | 3-F—Bn | 2-Cl, 4-F—Ph | Me | Me | | 333 |
| 312 | 4-F—Bn | 2-Cl, 4-F—Ph | Me | Me | | 333 |
| 313 (Ex. 41) | 2,4-di-F—Bn | 2-Cl, 4-F—Ph | Me | Me | | 351 |
| 344 | 4-Cl—Bn | 2-Cl, 4-F—Ph | Me | Me | | 349 |
| 424 | 4-Cl—Ph | 2,6-di-F, 4-MeO—Ph | Me | Br | 125-126 | |
| 431 | 2,6-di-F, 4-MeO—Ph | 4-I—Ph | Me | MeO | 122-124 | |
| 432 | 2-Cl, 4-F—Ph | 4-I—Ph | Me | MeO | 112-114 | |
| 578 | 2,6-di-F, 4-MeO—Ph | 4-Cl—Ph | Me | MeO | | 365 |
| 579 | 2,6-di-F, 4-MeO—Ph | 4-F—Ph | Me | MeO | | 349 |
| 580 | 2,6-di-F, 4-MeO—Ph | 4-MeO—Ph | Me | MeO | | 361 |

**See Index Table D for 1H NMR data.

INDEX TABLE C

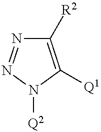

| Cmpd. | Q¹ | Q² | R² | AP⁺ (M + 1) |
|---|---|---|---|---|
| 24 (Ex. 42) | 2,4-di-F—Ph | 4-Cl—Ph | Cl | 326 |
| 30 | 2,4-di-F—Ph | 4-Cl—Ph | Me | 306 |
| 474 | 4-Cl—Ph | 2,6-di-F, 4-MeO—Ph | Br | 402 |
| 478 | 4-Cl—Ph | 2,6-di-F, 4-MeO—Ph | Cl | 356 |
| 595 | 4-Cl—Ph | 2,6-di-F, 4-MeO—Ph | Me | 336 |
| 626 | 2,6-di-F, | 4-Cl—Ph | Cl | 356 |
| | 4-MeO—Ph | | | |
| 627 | 2,6-di-F, | 4-Cl—Ph | Br | 402 |
| | 4-MeO—Ph | | | |
| 628 | 2,6-di-F, | 4-Cl—Ph | Me | 336 |
| | 4-MeO—Ph | | | |
| 644 | 4,6-di-Me- | 3,5-di-Cl—Ph | H | 320 |
| | 2-pyrimidinyl | | | |

INDEX TABLE D

| Cmpd. | ¹H NMR Data (CDCl₃ solution unless indicated otherwise)^{a} |
|---|---|
| 26 | δ 7.38-7.32 (m, 3H), 7.22-7.17 (m, 2H), 7.05-6.92 (m, 1H), 6.83-6.70 (m, 2H), 3.79 (s, 3H), |
| | 2.24 (s, 3H). |
| 31 | δ 7.30-7.25 (m, 3H), 7.20-7.11 (m, 3H), 7.08-7.00 (m, 1H), 6.90-6.83 (m, 1H), 3.82 (s, 3H), |
| | 2.17 (s, 3H). |
| 32 | δ 7.43 (t, 1H), 7.03 (dd, 1H), 6.95 (d, 1H), 6.43 (d, 2H), 3.78 (s, 3H). |
| 45 | δ 7.36-7.30 (m, 2H), 7.08-7.01 (m, 2H), 6.66-6.57 (m, 2H), 2.30 (s, 3H), 2.14 (s, 3H). |
| 165 | δ 7.34 (d, 2H), 7.30-7.21 (m, 1H), 7.17 (d, 2H), 7.93-7.82 (m, 2H), 4.08 (q, 2H), 1.43 (t, 3H). |
| 266 | δ 7.55 (s, 1H), 7.29-7.20 (m, 4H), 6.90 (d, 2H), 4.96 (s, 2H). |
| 267 | δ 7.12 (d, 2H), 7.12-7.01 (m, 1H), 6.98 (s, 1H), 6.81-6.72 (m, 3H), 4.99 (s, 2H), 2.82 (s, 6H). |
| 268 | δ 7.45 (s, 1H), 7.23 (d, 2H), 6.92 (d, 2H), 6.55-6.48 (m, 2H), 4.92 (s, 2H), 3.82 (s, 3H). |
| 269 | δ 7.13 (d, 2H), 6.88 (s, 1H), 6.75 (d, 2H), 6.44-6.38 (m, 2H), 4.94 (s, 2H), 3.77 (s, 3H), 2.77 (s, 6H). |
| 270 | δ 7.50 (s, 1H), 7.25 (d, 2H), 7.04-6.95 (m, 1H), 6.93-6.88 (m, 3H), 4.94 (s, 2H), 3.88 (s, 3H). |
| 271 | δ 7.11 (d, 2H), 6.86 (s, 1H), 6.88-6.73 (m, 4H), 4.98 (s, 2H), 3.83 (s, 3H), 2.79 (s, 6H). |
| 272 | δ 7.48 (s, 1H), 7.39 (d, 2H), 7.32-7.18 (m, 4H), 6.95 (s, 1H), 6.83 (d. 1H), 5.01 (s, 2H). |
| 273 | δ 7.26 (d, 2H), 7.20-7.15 (m, 4H), 6.91 (s, 1H), 6.86 (s, 1H), 6.73 (d, 1H), 5.07 (s, 2H), 2.77 (s, 6H). |
| 274 | δ 7.44 (s, 1H), 7.08 (d, 2H), 6.92 (d, 2H), 6.86-6.48 (m, 2H), 4.88 (s, 2H), 3.83 (s, 3H), 2.30 (s, 3H). |
| 275 | δ 6.97 (d, 2H), 6.87 (s, 1H), 6.73 (d, 2H), 6.43-6.35 (m, 2H), 4.94 (s, 2H), 3.76 (s, 3H), 2.76 (s, |
| | 6H), 2.24 (s, 3H). |
| 276 | δ 9.71 (s, 1H), 7.19-7.17 (m, 2H), 7.06 (t, J = 10.0 Hz, 2H), 6.44 (s, 1H), 6.42 (s, 1H), 3.78 (s, 3H). |
| 285 | δ 7.34-7.20 (m, 3H), 6.96-6.88 (m, 1H), 6.80 (m, 2H), 5.00 (s, 2H). |
| 288 | δ 7.20 (d, 2H), 6.99-7.08 (m, 1H), 6.93-6.80 (m, 3H), 4.99 (s, 2H), 3.88 (s, 3H). |
| 298 | δ 7.26-7.25 (m, 2 H), 7.07 (t, J = 10.0 Hz, 2H), 6.42 (s, 1H), 6.39 (s, 1H), 4.54 (d, J = 4.0 Hz, |
| | 2H), 4.13 (t, J = 8.0 Hz, 1H), 3.77 (s, 3H). |
| 320 | δ 8.36 (d, 1H), 8.15 (s, 1H), 7.60 (dd, 1H), 7.36 (m, 1H), 6.94 (m, 2H), 6.86 (d, 1H), 2.21 (2, 3H). |
| 321 | δ 8.34 (m, 1H), 7.74 (d, 1H), 7.29 (m, 2H), 6.84 (t, 2H), 2.17 (s, 3H). |
| 322 | δ 8.50 (d, 1H), 8.45 (s, 1H), 7.44 (t, 1H), 7.30 (t, 1H), 7.04 (dd, 1H), 4.50 (s, 2H). |
| 323 | δ 8.45 (s, 1H), 8.38 (d, 1H), 7.42 (t, 1H), 7.02 (m, 2H), 6.88 (d, 1H), 2.23 (s, 3H). |
| 330 | δ 7.73 (s, 1H), 7.36 (d, J = 8.0 Hz, 2H), 7.25-7.18 (m, 1H), 7.10 (d, J = 8.0 Hz, 2H), |
| | 6.89-6.83 (m, 1H). |
| 331 | δ 7.25-7.23 (m, 2H), 7.08 (t, J = 8.0 Hz, 2H), 6.43 (s, 1H), 6.41 (s, 1H), 5.25 (s, 1H), 5.13 (s, |
| | 1H), 3.78 (s, 3H). |
| 332 | δ 7.22 (s, 1H), 7.21-7.13 (m, 3H), 7.05-7.09 (t, 2H), 6.88-6.82 (t, 1H). |
| 342 | δ 7.75 (s, 1H), 7.45-7.41 (t, J = 8.0 Hz, 1H), 7.29-7.23 (m, 1H), 7.01-6.98 (dd, J = 8.0 Hz, |
| | 4.0 Hz, 1H), 6.93-6.86 (m, 2H). |
| 343 | δ 7.73 (s, 1H), 7.34 (d, J = 8.0 Hz, 2H), 7.23-7.20 (m, 1H), 7.09 (d, J = 8.0 Hz, 2H), |
| | 7.02-6.97 (m, 1H) |
| 342 | δ 7.75 (s, 1H), 7.45-7.41 (t, J = 8.0 Hz, 1H), 7.29-7.23 (m, 1H), 6.98-7.01 (dd, J = 8.0 Hz, |
| | 4.0 Hz, 1H), 6.93-6.86 (m, 2 H). |
| 347 | δ 8.56 (d, 1H), 7.80 (dd, 1H), 7.63 (s, 1H), 7.40 (t, 1H), 7.10 (d, 1H), 6.98 (dd, 1H), 6.89 (m, |
| | 1H), 2.41 (s, 3H). |
| 349 | δ 11.0 (br s, 1H), 7.42 (s, 1H), 6.82-6.79 (m, 2H), 6.68 (t, J = 8.0 Hz, 2H), 6.07 (s, 1H), 6.05 (s, |
| | 1H), 3.35 (s, 3H). |
| 350 | δ 7.46-7.42 (t, J = 8.0 Hz, 1H), 7.24 (m, 1H), 7.06-7.03 (dd, J = 8.0, 4.0 Hz, 1H), 6.96-6.94 (d, |
| | J = 8.0 Hz, 1H), 6.85 (m, 1H). |
| 351 | δ 7.38 (d, J = 8.0 Hz, 2H), 7.23-7.15 (m, 1H), 7.13 (d, J = 8.0 Hz, 2H), 6.86-6.81 (m, 1H). |
| 352 | δ 7.20-7.10 (m, 3H), 7.11-7.07 (t, J = 8.0 Hz, 2H), 6.85-6.80 (m, 1H). |
| 357 | δ 8.54 (d, 1H), 7.82 (dd, 1H), 7.46 (t, 1H), 7.21 (d, 1H), 7.10 (dd, 1H), 6.97 (m, 1H), 4.70 (s, 2H). |
| 363 | δ 7.36-7.22 (m, 1H), 7.08-6.87 (m, 4H), 6.63-6.55 (m, 2H), 2.67 (s, 6H). |
| 374 | δ 7.36 (d, J = 8.0 Hz, 2H), 7.17-7.13 (m, 3H), 6.97-6.93 (m, 1H). |
| 375 | δ 7.61 (s, 1H), 7.55 (t, 1H), 7.36 (t, 1H), 7.00 (m, 3H), 6.91 (m, 1H), 2.42 (s, 3H), 2.39 (s, 3H). |
| 376 | δ 8.80 (s, 1H), 7.74 (s, 1H), 7.42 (t, 1H), 6.98 (dd, 1H), 6.90 (m, 1H), 2.26 (s, 3H). |
| 377 | δ 7.72 (s, 1H), 7.46 (t, 1H), 7.01 (dd, 1H), 6.92 (m, 1H), 2.27 (s, 3H). |
| 378 | δ 7.51 (t, 1H), 7.40 (t, 1H), 7.08 (dd, 1H), 6.96 (m, 3H), 2.39 (s, 3H), 2.33 (s, 3H). |
| 379 | δ 7.57 (t, 1H), 7.44 (t, 1H), 7.11 (m, 2H), 7.05 (d, 1H), 7.00 (m, 1H), 4.73 (s, 2H), 2.37 (s, 3H). |
| 380 | δ 8.75 (s, 1H), 7.45 (t, 1H), 7.03 (dd, 1H), 6.93 (m, 1H), 2.22 (s, 3H). |
| 381 | δ 7.50 (t, 1H), 7.05 (dd, 1H), 6.95 (m, 1H), 2.24 (s, 3H). |
| 382 | δ 7.46-7.42 (t, J = 8.0 Hz, 1H), 7.22 (m, 1H), 7.06-7.03 (dd, J = 8.0, 4.0 Hz, 1H), 6.97-6.94 (dd, |
| | J = 8.0, 4.0 Hz, 1H), 6.88-6.85 (m, 1H). |
| 389 | δ 7.22-7.18 (m, 3H), 7.09-7.07 (t, J = 8.0 Hz, 2H), 6.84-6.79 (m, 1H). |
| 405 | δ 7.19 (d, 2H), 7.06 (d, 2H), 7.04 (m, 1H), 3.87 (s, 3H), 2.35 (s, 3H). |
| 409 | δ 7.74 (s, 1H), 7.43-7.39 (t, J = 8.0 Hz, 3H), 7.23-7.22 (m, 1H), 7.05-7.01 (m, 1H), |
| | 7.00-6.97 (dd, J = 8.0, 4.0 Hz, 1H), 6.92-6.89 (dd, J = 8.0, 4.0 Hz, 1H). |
| 410 | δ 7.71 (s, 1H), 7.20-7.16 (m, 1H), 7.14 (d, J = 8.0 Hz, 2H), 7.02 (d, J = 8.0 Hz, 2H), |
| | 7.01-6.94 (m, 1H), 2.34 (s, 3H). |
| 411 | δ 7.72 (s, 1H), 7.26-7.15 (m, 3H), 7.02 (d, J = 8.0 Hz, 2H), 6.86-6.81 (m, 1H), 2.35 (s, 3H). |
| 412 | δ 7.34 (d, J = 8.0 Hz, 2H), 7.18-7.13 (m, 1H), 7.11 (d, J = 8.0 Hz, 2H), 6.82-6.77 (m, 1H), |
| | 2.81 (s, 3H). |
| 413 | δ 7.17-7.03 (m, 5H), 6.82-6.77 (m, 1H), 2.18 (s, 3 H). |
| 414 | δ 7.32 (d, J = 8.0 Hz, 2H), 7.17-7.12 (m, 1H), 7.10 (d, J = 8.0 Hz, 2H), 6.94-6.89 (m, 1H), |
| | 2.13 (s, 3H). |
| 415 | δ 7.42-7.40 (t, J = 8.0 Hz, 1H), 7.14-7.12 (m, 1H), 6.97-6.95 (d, J = 8.0 Hz, 1H), 6.89-6.87 (d, J = 8.0 Hz, |
| | 1H), 6.80 (m, 1H), 2.33 (s, 3H), 2.16 (s, 3H). |
| 416 | δ 7.19 (d, J = 8.0 Hz, 2H), 7.02-6.97 (m, 1H), 6.91 (d, J = 8.0 Hz, 2H), 6.67-6.63 (m, 1 H), |
| | 1.97 (s, 3H), 1.88 (s, 3H). |
| 417 | δ 7.11-7.03 (m, 5H), 6.79-6.76 (m, 1H), 2.32 (s, 3H), 2.17 (s, 3H). |
| 434 | δ 8.00 (d, 1H), 7.48 (t, 1H), 7.34 (dd, 1H), 7.03 (dd, 1H), 6.95 (m, 1H), 6.70 (d, 1H), 3.92 (s, |
| | 3H). |
| 437 | δ 7.47 (t, 1H), 7.10 (dd, 1H), 7.01 (m, 1H), 6.87 (s, 2H), 3.80 (s, 3H). |
| 438 | δ 7.39 (t, 1H), 7.06 (d, 1H), 7.02 (dd, 1H), 6.92 (m, 1H), 6.74 (d, 1H), 3.70 (s, 3H). |
| 450 | δ 7.71 (s, 1 H), 7.15 (m, 2 H), 7.05 (m, 2 H), 6.95 (m, 1 H), 6.82 (m, 1 H), 3.85 (s, 3H). |
| 453 | δ 7.43-7.20 (m, 3H), 7.09 (d, 1H), 6.42 (d, 2H), 3.78 (s, 3H). |
| 459 | δ 7.44-7.40 (t, J = 8.0 Hz, 1H), 7.25-7.16 (m, 1H), 7.05 (d, J = 8.0 Hz, 1H), 6.97-6.95 (m, 2H). |
| 487 | δ 8.40 (m, 1H), 7.85 (dd, 1H), 7.61 (d, 1H), 7.45 (t, 1H), 7.08 (dd, 1H), 6.97 (m, 1H). |
| 488 | δ 8.57 (br s, 1H), 7.77 (m, 2H), 6.44 (d, 2H), 3.79 (s, 3H). |
| 460 | δ 7.16 (d, J = 8.0 Hz, 2H), 7.15-7.12 (m, 1H), 7.06 (d, J = 8.0 Hz, 2H), 6.93-6.92 (m, 1H), |
| | 2.34 (s, 3H). |
| 461 | δ 7.17 (d, J = 8.0 Hz, 2H), 7.15-7.10 (m, 1H), 7.05 (d, J = 8.0 Hz, 2H), 6.82-6.78 (m, 1H), |
| | 2.36 (s, 3H). |
| 462 | δ 7.19-7.16 (m, 2H), 7.11-7.07 (t, J = 8.0 Hz, 2H), 6.67-6.63 (t, J = 8.0 Hz, 2H). |
| 463 | δ 7.45-7.40 (t, J = 8.0 Hz, 1H), 7.21-7.06 (m, 1H), 7.05 (d, J = 8.0 Hz, 1H), 6.96-6.94 (m, 2H). |
| 646 | δ 7.16 (d, J = 8.0 Hz, 2 H,), 7.15-7.10 (m, 1H), 7.06 (d, J = 8.0 Hz, 2H), 6.95-6.92 (m, 1H), |
| | 2.34 (s, 3H). |
| 465 | δ 7.18 (d, J = 8.0 Hz, 2H), 7.15-7.10 (m, 1H), 7.05 (d, J = 8.0 Hz, 2H), 6.81-6.78 (m, 1H), |
| | 2.36 (s, 3H). |
| 471 | δ 7.35 (d, 2 H), 7.14 (d, 2 H), 6.95 (m, 1 H), 6.79 (m, 1 H), 3.83 (s, 3 H). |
| 489 | δ 7.39-7.35 (t, J = 8.0 Hz, 1H), 7.12-7.08 (m, 2H), 6.95-6.91 (m, 1H), 6.86-6.79 (d, J = 8.0 Hz, |
| | 1H), 2.33 (s, 3H), 2.11 (s, 3H). |
| 490 | δ 7.14-7.12 (d, J = 8.0 Hz, 2H), 7.11-7.02 (m, 1H), 6.99-6.97 (d, J = 8.0 Hz, 2H), 6.77-6.72 (m, |
| | 2H), 2.34 (s, 3H), 2.16 (s, 3H). |
| 491 | δ 7.11-7.02 (m, 4 H), 6.62-6.50 (t, J = 8.0 Hz, 2H), 2.28 (s, 3H), 2.13 (s, 3H). |
| 492 | δ 7.15-7.13 (m, 2H), 7.05-7.03 (t, J = 8.0 Hz, 2H), 6.61-6.64 (t, J = 8.0 Hz, 2H), 2.15 (s, 3H). |
| 493 | δ 7.11-7.09 (d, J = 8.0 Hz, 2H), 7.14-7.01 (m, 1H), 6.99-6.97 (d, J = 8.0 Hz, 2H), 6.88-6.83 (m, |
| | 1H), 2.32 (s, 3H), 2.12 (s, 3H). |
| 494 | δ 7.40-7.36 (t, J = 8.0 Hz, 1H), 7.19 (m, 2H), 7.01 (d, J = 8.0 Hz, 1H), 6.98-6.90 (m, 2H), |
| | 2.13 (s, 3H). |
| 495 | δ 7.12 (d, J = 8.0 Hz, 2H), 7.10-7.05 (m, 1H), 7.03 (d, J = 8.0 Hz, 2H), 6.91-6.87 (m, 1H), |
| | 2.33 (s, 3H), 2.13 (s, 3H). |
| 505 | δ 7.15 (d, J = 8.0 Hz, 2H), 7.13-7.05 (m, 1H), 7.03 (d, J = 8.0 Hz, 2H), 6.80-6.75 (m, 1H), |
| | 2.35 (s, 3H), 2.17 (s, 3H). |
| 506 | δ 7.68 (s, 1H), 7.15 (d, J = 8.0 Hz, 2H), 7.03 (d, J = 8.0 Hz, 2H), 6.43 (d, J = 12.0 Hz, 2H), |
| | 3.78 (s, 3H), 2.35 (s, 3H). |
| 507 | δ 7.70 (s, 1H), 7.14 (d, J = 8.0 Hz, 2H), 7.01 (d, J = 8.0 Hz, 2H), 6.69-6.65 (t, J = 8.0 Hz, 2H), |
| | 2.38 (s, 3H). |
| 508 | δ 7.71 (s, 1H), 7.24-7.13 (m, 3H), 7.07-7.03 (t, J = 8.0 Hz, 1H), 6.98-6.96 (m, 1H). |
| 510 | δ 7.35 (d, 2 H), 7.14 (d, 2 H), 6.95 (m, 1 H), 6.80 (m, 1 H), 3.84 (s, 3 H). |
| 514 | δ 7.46 (d, 1H), 6.96 (d, 1H), 6.41 (m, 2H), 3.85 (s, 3H), 3.78 (s, 3H). |
| 521 | δ 7.12 (d, J = 8.0 Hz, 2H), 6.98 (d, J = 8.0 Hz, 2H), 6.36 (d, J = 8.0 Hz, 2H), 3.74 (s, 3H), |
| | 2.27 (s, 3H), 2.13 (s, 3H). |
| 522 | δ 7.11-7.00 (m, 4H), 6.37 (d, J = 8.0 Hz, 2H), 3.75 (s, 3H), 2.28 (s, 3H), 2.13 (s, 3H). |
| 523 | δ 7.19-7.17 (m, 2H), 7.05-7.09 (t, J = 8.0 Hz, 2H), 6.40 (d, J = 8.0 Hz, 2H), 3.71 (s, 3H). |
| 524 | δ 7.16 (d, J = 8.0 Hz, 2H,), 7.05 (d, J = 8.0 Hz, 2H), 6.39 (d, J = 8.0 Hz, 2H), 3.76 (s, 3H), |
| | 2.36 (s, 3H). |
| 574 | δ 7.41 (t, 1H), 7.11 (dd, 1H), 7.01 (m, 1H), 6.86 (s, 2H), 3.80 (s, 3H). |
| 575 | δ 7.42 (t, 1H), 7.01 (m, 2H), 6.90 (m, 2H), 3.75 (s, 3H). |
| 576 | δ 8.46 (m, 1H), 7.73 (m, 1H), 7.39 (m, 1H), 6.48 (m, 1H), 6.34 (m, 1H), 3.77 (s, 3H). |
| 577 | δ 8.25 (s, 1H), 7.59 (d, 1H), 7.32 (m, 1H), 7.19 (d, 1H), 6.86 (t, 2H), 2.36 (s, 3H). |
| 581 | δ 7.15-7.10 (m, 4H), 6.53-6.47 (m, 2H), 4.54 (s, 2H), 3.80 (s, 3H), 2.33 (s, 3H). |
| 592 | δ 8.24 (s, 1H), 7.54 (d, 1H), 7.39 (m, 2H), 6.91 (t, 2H). |
| 593 | δ 8.39 (s, 2H), 7.46 (t, 1H), 7.07 (dd, 1H), 6.94 (d, 1H). |
| 594 | δ 8.42 (s, 2H), 7.76 (s, 1H), 7.45 (t, 1H), 7.01 (dd, 1H), 6.90 (d, 1H). |
| 616 | δ 8.20 (dd, 1H), 7.54 (m, 1H), 7.31 (m, 1H), 6.93 (m, 2H), 6.81 (t, 1H), 3.81 (s, 3H). |
| 672 | δ 7.35 (d, 2H), 7.04 (d, 2H), 6.40 (m, 2H), 6.50 (m, 1H), 3.75 (s, 3H), 2.32 (s, 3H). |
| 686 | δ 7.35-7.29 (m, 1H), 7.08-6.92 (m, 3H), 6.56-6.50 (m, 2H), 4.54 (s, 2H), 3.82 (s, 3H). |
| 712 | δ 7.36-7.28 (m, 1H), 7.08-7.02 (m, 2H), 6.98-6.92 (m, 1H), 6.53 (d, 2H), 4.54 (s, 2H), 3.82 (s, 3H). |

### Biological Examples of the Invention

General protocol for preparing test suspensions for Tests A-J: The test compounds were first dissolved in acetone in an amount equal to 3% of the final volume and then suspended at the desired concentration (in ppm) in acetone and purified water (50/50 mix) containing 250 ppm of the surfactant Trem® 014 (polyhydric alcohol esters). The resulting test suspensions were then used in tests A-J. Spraying a 200 ppm test suspension to the point of run-off on the test plants was the equivalent of a rate of 500 g/ha. (An asterisk “*” next to the rating value indicates a 40 ppm test suspension.)

### Test A

Grape seedlings were inoculated with a spore suspension of Plasmopara viticola (the causal agent of grape downy mildew) and incubated in a saturated atmosphere at 20° C. for 24 h. After a short drying period, the grape seedlings were sprayed with the test suspension to the point of run-off and then moved to a growth chamber at 20° C. for 5 days, after which time the grape seedlings were placed back into a saturated atmosphere at 20° C. for 24 h. Upon removal, visual disease ratings were made.

### Test B

The test suspension was sprayed to the point of run-off on bentgrass (Agrostis sp.) seedlings. The following day the seedlings were inoculated with a bran and mycelial slurry of Rhizoctonia solani (the causal agent of turf brown patch) and incubated in a saturated atmosphere at 27° C. for 48 h, and then moved to a growth chamber at 27° C. for 6 days, after which time visual disease ratings were made.

### Test C

The test suspension was sprayed to the point of run-off on tomato seedlings. The following day the seedlings were inoculated with a spore suspension of Botrytis cinerea (the causal agent of tomato Botrytis) and incubated in saturated atmosphere at 20° C. for 48 h, and then moved to a growth chamber at 24° C. for 3 days, after which time visual disease ratings were made.

### Test D

The test suspension was sprayed to the point of run-off on wheat seedlings. The following day the seedlings were inoculated with a spore suspension of Alternaria solani (the causal agent of tomato early blight) and incubated in a saturated atmosphere at 27° C. for 48 h, and then moved to a growth chamber at 20° C. for 5 days, after which time visual disease ratings were made.

### Test E

The test suspension was sprayed to the point of run-off on tomato seedlings. The following day the seedlings were inoculated with a spore suspension of Phytophthora infestans (the causal agent of tomato late blight) and incubated in a saturated atmosphere at 20° C. for 24 h, and then moved to a growth chamber at 20° C. for 5 days, after which time visual disease ratings were made.

### Test F

The test suspension was sprayed to the point of run-off on wheat seedlings. The following day the seedlings were inoculated with a spore suspension of Septoria nodorum (the causal agent of wheat glume blotch) and incubated in a saturated atmosphere at 24° C. for 48 h, and then moved to a growth chamber at 20° C. for 9 days, after which time visual disease ratings were made.

### Test G

The test suspension was sprayed to the point of run-off on wheat seedlings. The following day the seedlings were inoculated with a spore suspension of Septoria tritici (the causal agent of wheat leaf blotch) and incubated in saturated atmosphere at 24° C. for 48 h, and then moved to a growth chamber at 20° C. for 19 days, after which time visual disease ratings were made.

### Test H

Wheat seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 2 days. At the end of this time the test suspension was sprayed to the point of run-off on the wheat seedlings, then the seedlings were moved to a growth chamber at 20° C. for 4 days, after which time visual disease ratings were made.

### Test I

The test suspension was sprayed to the point of run-off on wheat seedlings. The following day the seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 6 days, after which time visual disease ratings were made

### Test J

The test suspension was sprayed to the point of run-off on wheat seedlings. The following day the seedlings were inoculated with a spore dust of Erysiphe graminis f. sp. tritici, (the causal agent of wheat powdery mildew) and incubated in a growth chamber at 20° C. for 7 days, after which time visual disease ratings were made.

Results for Tests A-J are given in Table A. In the Table, a rating of 100 indicates 100% disease control and a rating of 0 indicates no disease control (relative to the controls). A dash (-) indicates no test results. All results are for 200 ppm except where followed by “*”, which indicates 40 ppm.

TABLE A

| Cmpd. | Test A | Test B | Test C | Test D | Test E | Test F | Test G | Test H | Test I | Test J |
|---|---|---|---|---|---|---|---|---|---|---|
| 1 | — | — | 99* | 94* | — | 0* | 93* | — | 16* | 99* |
| 2 | — | 65 | 99 | 100 | — | 100 | 100 | — | 100 | 100 |
| 3 | — | 99 | 99 | 100 | — | 99 | 100 | — | 99 | 100 |
| 4 | 0 | 18 | 67 | 0 | 0 | 0 | 93 | — | 79 | 94 |
| 5 | 0 | 38 | 98 | 99 | 0 | 0 | 100 | — | 88 | 99 |
| 6 | 0 | 99 | 99 | 100 | 0 | 95 | 100 | — | 100 | 100 |
| 7 | 0 | 81 | 100 | 100 | 0 | 0 | 99 | — | 98 | 99 |
| 8 | — | — | 99 | 8 | — | 0 | 68 | — | 74 | 97 |
| 9 | — | — | 97* | 100* | — | 0* | 33* | — | 68* | 94* |
| 10 | — | — | 99* | 99* | — | 0* | 94* | — | 97* | 100* |
| 11 | 6 | 99 | 100 | 100 | 0 | 99 | 99 | — | 100 | 100 |
| 12 | — | — | 87* | 100* | — | 94* | 96* | — | 99* | 100* |
| 13 | — | — | | 0 | — | 0 | 97 | — | 99 | 90 |
| 14 | — | — | | 99 | — | 0 | 53 | — | 96 | 75 |
| 15 | — | — | 98* | 100* | — | 0* | 84* | — | 97* | 95* |
| 16 | — | — | 97 | 8 | — | 92 | 3 | — | 41 | 0 |
| 17 | — | — | | 100 | — | 0 | 96 | — | 98 | 100 |
| 18 | — | — | 98 | 99 | — | 0 | 93 | — | 98 | 100 |
| 19 | — | — | 99 | 99 | — | 0 | 88 | — | 100 | 95 |
| 20 | 0 | 0 | 100 | 100 | 0 | 0 | 100 | — | 98 | 100 |
| 21 | 13 | 100 | 99 | 100 | 0 | 100 | 100 | — | 99 | 100 |
| 22 | — | — | 93 | 100 | — | 0 | 91 | — | 100 | 100 |
| 23 | — | — | 99 | 100 | — | — | 88 | — | 100 | 99 |
| 24 | — | — | 90 | 0 | — | 0 | 67 | — | 28 | 95 |
| 25 | — | — | 93 | 0 | — | 0 | 0 | — | 6 | 13 |
| 26 | — | — | 96 | 8 | — | 0 | 0 | — | 34 | 0 |
| 27 | — | — | 97 | 100 | — | 51 | 95 | — | 100 | 100 |
| 28 | — | — | 99 | 100 | — | 74 | 91 | — | 100 | 100 |
| 29 | — | — | 99* | 100* | — | 0* | 94* | — | 100* | 100* |
| 30 | — | — | 99 | 0 | — | 0 | 73 | — | 0 | 95 |
| 31 | — | — | 92 | 15 | — | 0 | 27 | — | 13 | 73 |
| 32 | — | — | 99 | 100 | — | 100 | 95 | — | 100 | 100 |
| 33 | — | — | 99 | 100 | — | 0 | 93 | — | 99 | 98 |
| 34 | — | — | 99* | 100* | — | 99* | 90* | 91* | 100* | 100* |
| 35 | 20 | 100 | 100 | 100 | 0 | 100 | 100 | — | 100 | 100 |
| 36 | — | — | 99 | 100 | — | 99 | 92 | — | 100 | 100 |
| 37 | — | — | 99* | 100* | — | 100* | 92* | — | 100* | 100* |
| 38 | — | — | 99* | 100* | — | 0* | 96* | — | 86* | 87* |
| 39 | — | — | 94* | 100* | — | 0 | 89* | — | 89* | 96* |
| 40 | — | — | 99* | 100* | — | 99* | 95* | — | 98* | 100* |
| 41 | — | — | 99 | 100 | — | 64 | 94 | — | 99 | 98 |
| 42 | — | — | 99* | 100* | — | 99* | 93* | — | 91* | 100* |
| 43 | — | — | 98* | 100* | — | 97* | 96* | — | 97* | 99* |
| 44 | — | — | 99* | 100* | — | 40* | 92* | — | 99* | 98* |
| 45 | — | — | 100 | 98 | — | 100 | — | 99 | 100 | 100 |
| 46 | — | — | 86* | 97* | — | 0* | 83* | — | 26* | 78* |
| 47 | — | — | 94 | 42 | — | 0 | 77 | — | 54 | 93 |
| 48 | — | — | 99 | 100 | — | 100 | 94 | — | 100 | 100 |
| 49 | — | — | 98 | 100 | — | 100 | 94 | — | 99 | 99 |
| 50 | — | — | 98 | 100 | — | 100 | 92 | — | 100 | 100 |
| 51 | — | — | 94 | 100 | — | 100 | 94 | — | 97 | 99 |
| 52 | — | — | 84 | 100 | — | 0 | 97 | — | 99 | 100 |
| 53 | — | — | 89 | 100 | — | 0 | 95 | — | 100 | 99 |
| 54 | — | — | 92 | 24 | — | 0 | 87 | — | 99 | 91 |
| 55 | — | — | 94* | 100* | — | 98* | 93* | — | 99* | 100* |
| 56 | — | — | 96* | 100* | — | 60* | 96* | — | 94* | 99 |
| 57 | — | — | 84 | 99 | — | 0 | 87 | — | 96 | 100 |
| 58 | — | — | 98 | 100 | — | 51 | 79 | — | 99 | 100 |
| 59 | — | — | 93* | 100* | — | 82* | 97* | — | 86* | 97* |
| 60 | — | — | 81 | 0 | — | 0 | 2 | — | 0 | 0 |
| 61 | — | — | 40 | 0 | — | 0 | 60 | — | 92 | 99 |
| 62 | — | — | 94 | 0 | — | 0 | 5 | — | 0 | 81 |
| 63 | — | — | 0 | 0 | — | 0 | 33 | — | 41 | 100 |
| 64 | — | — | 99 | 100 | — | 95 | 95 | — | 100 | 96 |
| 65 | — | — | 0 | 100 | — | 0 | 95 | — | 41 | 95 |
| 66 | — | — | 99 | 100 | — | 99 | 94 | — | 99 | 99 |
| 67 | 7 | 98 | 44 | 99 | 0 | 0 | 80 | — | 0 | 99 |
| 68 | — | — | 0 | 0 | — | 0 | 40 | — | 9 | 99 |
| 69 | — | — | 74 | 99 | — | 0 | 25 | — | 0 | 97 |
| 70 | — | — | 71 | 0 | — | 0 | 0 | — | 0 | 0 |
| 71 | — | — | 47 | 33 | — | 0 | 2 | — | 9 | 56 |
| 72 | — | — | 96 | 100 | — | 92 | 97 | — | 99 | 100 |
| 73 | — | — | 100 | 100 | — | 60 | 87 | — | 100 | 100 |
| 74 | — | — | 98 | 96 | — | 31 | 98 | — | 99 | 99 |
| 75 | — | — | 98 | 93 | — | 0 | 95 | — | 77 | 98 |
| 76 | — | — | 37 | 99 | — | 60 | 97 | — | 99 | 100 |
| 77 | — | — | 98 | 100 | — | 82 | 97 | — | 99 | 100 |
| 78 | — | — | 91 | 100 | — | 0 | 92 | — | 99 | 100 |
| 79 | — | — | 53 | 97 | — | 0 | 95 | — | 92 | 100 |
| 80 | — | — | 90 | 100 | — | 100 | 98 | — | 100 | 100 |
| 81 | — | — | 81 | 100 | — | 69 | 93 | — | 98 | 100 |
| 82 | — | — | 90 | 100 | — | 100 | 91 | — | 99 | 100 |
| 83 | — | — | 97 | 100 | — | 99 | 98 | — | 100 | 100 |
| 84 | — | — | 0 | 99 | — | 0 | 88 | — | 91 | 99 |
| 85 | — | — | 73 | 100 | — | 11 | 98 | — | 96 | 100 |
| 86 | — | — | 0 | 0 | — | 0 | 93 | — | 27 | 99 |
| 87 | — | — | 0 | 0 | — | 0 | 84 | — | 27 | 99 |
| 88 | — | — | 75 | 0 | — | 0 | 0 | — | 68 | 99 |
| 89 | — | — | 0 | 0 | — | 0 | 3 | — | 85 | 98 |
| 90 | — | — | 0 | 0 | — | 0 | 0 | — | 54 | 0 |
| 91 | — | — | 98 | 100 | — | 100 | 99 | — | 96 | 100 |
| 92 | — | — | 76 | 100 | — | 0 | 98 | — | 89 | 100 |
| 93 | — | — | 97 | 100 | — | 100 | 99 | — | 98 | 100 |
| 94 | — | — | 56 | 53 | — | 0 | 95 | — | 99 | 100 |
| 95 | — | — | 96 | 97 | — | 95 | 95 | — | 100 | 100 |
| 96 | — | — | 98* | 100* | — | 97* | 97* | — | 97* | 99* |
| 97 | — | — | 94 | 97 | — | 0 | 95 | — | 91 | 99 |
| 98 | — | — | 0* | 0* | — | 0* | 27* | — | 0* | 0* |
| 99 | — | — | 0 | 0 | — | 0 | 25 | — | 91 | 0 |
| 100 | — | — | 97 | 93 | — | 99 | 97 | — | 98 | 99 |
| 101 | — | — | 88 | 0 | — | 0 | 97 | — | 100 | 99 |
| 102 | — | — | 98 | 0 | — | 55 | 97 | — | 99 | 99 |
| 103 | — | — | 79 | 92 | — | 90 | 98 | — | 100 | 99 |
| 104 | — | 0 | 92 | 98 | 0 | 0 | 32 | — | 0 | 95 |
| 105 | — | — | 98 | 99 | — | 94 | 94 | — | 99 | 100 |
| 106 | — | — | 100 | 99 | — | 64 | 93 | — | 99 | 95 |
| 107 | — | — | 96 | 100 | — | 100 | 96 | — | 100 | 100 |
| 108 | — | — | 100* | 100* | — | 0* | 74* | — | 98* | 99* |
| 109 | — | — | 94 | 57 | — | 0 | 94 | — | 68 | 63 |
| 110 | — | — | 94 | 99 | — | 100 | 95 | — | 97 | 96 |
| 111 | — | 81 | 99 | 99 | 0 | 100 | 100 | — | 89 | 99 |
| 112 | — | — | 99 | 100 | — | 89 | 98 | — | 99 | 99 |
| 113 | — | — | 98 | 99 | — | 97 | 95 | — | 97 | 97 |
| 114 | — | — | 93 | 0 | — | 0 | 95 | — | 41 | 72 |
| 115 | — | 99 | 99 | 99 | 0 | 98 | 99 | — | 98 | 100 |
| 116 | — | — | 0* | 0* | — | 0* | 0* | — | 0* | 0* |
| 117 | — | — | 96 | 100 | — | 99 | 97 | — | 99 | 100 |
| 118 | — | — | 99 | 100 | — | 60 | 94 | — | 99 | 100 |
| 119 | — | — | 96 | 80 | — | 40 | 91 | — | 80 | 0 |
| 120 | — | — | 98 | 0 | — | 0 | 96 | — | 90 | 92 |
| 121 | — | — | 99 | 100 | — | 0 | 85 | — | 89 | 97 |
| 122 | — | — | 98 | 100 | — | 100 | 96 | — | 100 | 100 |
| 123 | — | — | 99 | 99 | — | 0 | 48 | — | 90 | 94 |
| 124 | — | — | 98 | 97 | — | 0 | 90 | — | 95 | 98 |
| 125 | — | — | 98 | 100 | — | 0 | 92 | — | 91 | 100 |
| 126 | — | — | 97 | 99 | — | 69 | 96 | — | 99 | 99 |
| 127 | — | — | 99 | 100 | — | 98 | 95 | — | 100 | 100 |
| 128 | — | — | 97 | 96 | — | 96 | 93 | — | 97 | 100 |
| 129 | — | — | 99 | 100 | — | 0 | 89 | — | 74 | 95 |
| 130 | — | — | 98 | 100 | — | 0 | 85 | — | 99 | 96 |
| 131 | — | — | 90* | 99* | — | 0* | 85* | — | 0* | 48* |
| 132 | — | — | 94 | 98 | — | 0 | 92 | — | 74 | 97 |
| 133 | — | — | 0 | 0 | — | 0 | 0 | — | 0 | 0 |
| 134 | — | — | 97 | 100 | — | 99 | 93 | — | 99 | 100 |
| 135 | — | — | 99 | 100 | — | 0 | 93 | — | 100 | 100 |
| 136 | — | — | 99 | 100 | — | 99 | 96 | — | 100 | 100 |
| 137 | — | — | 94 | 100 | — | 0 | 98 | — | 55 | 97 |
| 138 | — | — | 98 | 100 | — | 92 | 99 | — | 100 | 100 |
| 139 | — | — | 99 | 100 | — | 0 | 75 | — | 98 | 98 |
| 140 | — | — | 0 | 0 | — | 0 | 93 | — | 55 | 39 |
| 141 | — | — | 99 | 100 | — | 0 | 91 | — | 99 | 100 |
| 142 | — | — | 99 | 100 | — | 97 | 94 | — | 100 | 100 |
| 143 | — | — | 99 | 100 | — | 100 | 100 | — | 100 | 100 |
| 144 | — | — | 100 | 100 | — | 100 | 100 | — | 100 | 100 |
| 145 | — | — | 99 | 100 | — | 100 | 100 | — | 100 | 100 |
| 146 | — | — | 99 | 100 | — | 100 | 100 | — | 100 | 100 |
| 147 | — | — | 91 | 100 | — | 82 | 100 | — | 100 | 99 |
| 148 | — | — | 99 | 100 | — | 99 | 100 | — | 100 | 100 |
| 149 | — | — | 53 | 0 | — | 98 | 96 | — | 95 | 95 |
| 150 | — | — | 99 | 100 | — | 60 | 98 | — | 97 | 100 |
| 151 | — | — | 97* | 100* | — | 69* | 99* | — | 89* | 96* |
| 152 | — | — | 99 | 99 | — | 69 | 96 | — | 89 | 99 |
| 153 | — | — | 98 | 100 | — | 69 | 96 | — | 99 | 100 |
| 154 | — | — | 99 | 100 | — | 0 | 93 | — | 86 | 99 |
| 155 | — | — | 99 | 100 | — | 87 | 95 | — | 99 | 100 |
| 156 | — | — | 98* | 100* | — | 99* | 97* | — | 97* | 99* |
| 157 | — | — | 97 | 99 | — | 98 | 97 | — | 100 | 100 |
| 158 | — | — | 99 | 100 | — | 96 | 97 | — | 100 | 100 |
| 159 | — | — | 99 | 100 | — | 99 | 97 | — | 100 | 100 |
| 160 | — | — | 91 | 93 | — | 0 | 93 | — | 28 | 98 |
| 161 | — | — | 99 | 71 | — | 0 | 1 | — | 80 | 100 |
| 162 | — | — | 88* | 97* | — | 0* | 93* | — | 23* | 94* |
| 163 | — | — | 0* | 0* | — | 0* | 10* | — | 0* | 0* |
| 164 | — | — | 91* | 88* | — | 0* | 96* | — | 19* | 94* |
| 165 | — | — | 0 | 0 | — | 0 | 93 | — | 27 | 93 |
| 166 | — | — | 96 | 97 | — | 40 | 96 | — | 91 | 100 |
| 167 | — | — | 99* | 100* | — | 0* | 95* | — | 0* | 98* |
| 168 | — | — | 97 | 99 | — | 84 | 98 | — | 100 | 100 |
| 169 | — | — | 99 | 100 | — | 40 | 96 | — | 88 | 99 |
| 170 | — | — | 97 | 98 | — | 0 | 95 | — | 99 | 99 |
| 171 | — | — | 99 | 100 | — | 100 | 98 | — | 98 | 99 |
| 172 | — | — | 99 | 99 | — | 100 | 98 | — | 99 | 99 |
| 173 | — | — | 98* | 0* | — | 0* | 90* | — | 41* | 0* |
| 174 | — | — | 98 | 99 | — | 82 | 98 | — | 100 | 100 |
| 175 | — | — | 97 | 97 | — | 82 | 98 | — | 100 | 99 |
| 176 | — | — | 96 | 91 | — | 0 | 87 | — | 99 | 99 |
| 177 | — | — | 88 | 0 | — | 0 | 79 | — | 99 | 99 |
| 178 | 9 | 0 | 99 | 99 | — | 0 | 100 | — | 74 | 100 |
| 179 | 0 | 0 | 99 | 9 | 0 | 0 | 98 | — | 99 | 99 |
| 180 | 0 | 88 | 99 | 99 | — | 0 | 98 | — | 99 | 100 |
| 181 | — | 24 | 99 | 0 | — | 0 | 94 | — | 98 | 99 |
| 182 | — | — | 99 | 100 | — | 73 | 96 | — | 97 | 99 |
| 183 | — | — | 99* | 91* | — | 0* | 85* | — | 66* | 48* |
| 184 | — | — | 99 | 100 | — | 0 | 97 | — | 100 | 100 |
| 185 | — | — | 99 | 100 | — | 78 | 94 | — | 100 | 99 |
| 186 | — | — | 98 | 88 | — | 0 | 97 | — | 100 | 100 |
| 187 | — | — | 99 | 100 | — | 94 | 97 | — | 100 | 100 |
| 188 | — | — | 98 | 100 | — | 0 | 97 | — | 100 | 99 |
| 189 | — | — | 98 | 100 | — | 51 | 97 | — | 100 | 99 |
| 190 | — | — | 97 | 100 | — | 0 | 93 | — | 100 | 100 |
| 191 | 0 | 0 | 99 | 26 | 29 | 0 | 97 | — | 80 | 93 |
| 192 | 0 | 21 | 99 | 16 | 0 | 0 | 94 | — | 84 | 99 |
| 193 | — | — | 99 | 100 | — | 0 | 98 | — | 97 | 98 |
| 194 | — | — | 99 | 95 | — | 73 | 97 | — | 100 | 96 |
| 195 | — | — | 99 | 100 | — | 64 | 94 | — | 100 | 94 |
| 196 | — | — | 99 | 100 | — | 0 | 92 | — | 100 | 97 |
| 197 | — | — | 99 | 99 | — | 0 | 93 | — | 100 | 100 |
| 198 | — | — | 98 | 99 | — | 0 | 90 | — | 100 | 95 |
| 199 | — | — | 92 | 100 | — | 100 | 96 | — | 98 | 95 |
| 200 | — | — | 0 | 0 | — | 0 | 45 | — | 0 | 21 |
| 201 | — | — | 99 | 100 | — | 100 | 98 | — | 96 | 100 |
| 202 | — | — | 91 | 61 | — | 98 | 97 | — | 98 | 100 |
| 203 | — | — | 87 | 98 | — | 87 | 97 | — | 100 | 99 |
| 204 | — | — | 90* | 91* | — | 0* | 90* | — | 18* | 26* |
| 205 | — | — | 99 | 100 | — | 100 | 98 | — | 100 | 100 |
| 206 | — | — | 95 | 99 | — | 0 | 98 | — | 100 | 99 |
| 207 | — | — | 93 | 99 | — | 80 | 98 | — | 100 | 99 |
| 208 | — | — | 92 | 39 | — | 0 | 98 | — | 98 | 99 |
| 209 | — | — | 88 | 99 | — | 0 | 96 | — | 99 | 99 |
| 210 | — | — | 97 | 66 | — | 0 | 91 | — | 98 | 99 |
| 211 | — | — | 74 | 0 | — | 0 | 0 | — | 37 | 86 |
| 212 | — | — | 40* | 20* | — | 0* | 0* | — | 0* | 0* |
| 213 | — | — | 63 | 0 | — | 0 | 96 | — | 100 | 42 |
| 214 | — | — | 100* | 99* | — | 97* | 94* | — | 97* | 98* |
| 215 | — | — | 98 | 85 | — | 0 | 96 | — | 68 | 91 |
| 216 | — | — | 99* | 100* | — | 0* | 96* | — | 93* | 94* |
| 217 | — | — | 33* | 57* | — | 0* | 13* | — | 0* | 0* |
| 218 | — | — | 99* | 99* | — | 0* | 91* | — | 80* | 73* |
| 219 | — | — | 98 | 97 | — | 51 | 96 | — | 99 | 99 |
| 220 | — | — | 98 | 99 | — | 0 | 97 | — | 99 | 100 |
| 221 | — | — | 99 | 99 | — | 0 | 95 | — | 97 | 98 |
| 222 | — | — | 99 | 99 | — | 0 | 99 | — | 97 | 98 |
| 223 | — | — | 99 | 100 | — | 60 | 98 | — | 99 | 100 |
| 224 | — | — | 93 | 51 | — | 0 | 90 | — | 80 | 98 |
| 225 | — | — | 96 | 100 | — | 0 | 98 | — | 99 | 100 |
| 226 | — | — | 76 | 67 | — | 0 | 88 | — | 0 | 95 |
| 227 | — | — | 98 | 96 | — | 0 | 94 | — | 99 | 99 |
| 228 | — | — | 96 | 99 | — | 0 | 98 | — | 94 | 99 |
| 229 | — | — | 98 | 99 | — | 0 | 98 | — | 89 | 91 |
| 230 | — | — | 99 | 97 | — | 0 | 99 | — | 94 | 99 |
| 231 | — | — | 87 | 61 | — | 100 | 99 | — | 96 | 98 |
| 232 | — | — | 0 | 0 | — | 0 | 95 | — | 9 | 0 |
| 233 | — | — | 96* | 88* | — | 0* | 95* | — | 41* | 94* |
| 234 | — | — | 95 | 99 | — | 0 | 98 | — | 55 | 98 |
| 235 | — | — | 99 | 99 | — | 92 | 99 | — | 97 | 99 |
| 236 | — | — | 100 | 100 | — | 99 | 96 | — | 100 | 100 |
| 237 | — | — | 100 | 100 | — | 100 | 98 | — | 100 | 100 |
| 238 | — | — | 99 | 99 | — | 0 | 93 | — | 96 | 100 |
| 239 | — | — | 100 | 100 | — | 100 | 96 | — | 99 | 100 |
| 240 | — | — | 100 | 100 | — | 100 | 97 | — | 100 | 100 |
| 241 | — | — | 100 | 100 | — | 100 | 96 | — | 96 | 93 |
| 242 | — | — | 99* | 95* | — | 51* | 95* | — | 89* | 92* |
| 243 | — | — | 99 | 100 | — | 98 | 99 | — | 100 | 98 |
| 244 | — | — | 100 | 100 | — | 99 | 96 | — | 99 | 100 |
| 245 | — | — | 100 | 99 | — | 100 | 97 | — | 95 | 99 |
| 246 | — | — | 100 | 100 | — | 95 | 93 | — | 100 | 99 |
| 247 | — | — | 100 | 100 | — | 100 | 97 | — | 100 | 100 |
| 248 | — | — | 99* | 47* | — | 0* | 92* | — | 68* | 92* |
| 249 | — | — | 100* | 99* | — | 73* | 93* | — | 74* | 81* |
| 250 | — | — | 99 | 100 | — | 0 | 83 | — | 99 | 94 |
| 251 | — | — | 99 | 0 | — | 0 | 81 | — | 9 | 0 |
| 252 | — | — | 15 | 0 | — | 0 | 13 | — | 0 | 0 |
| 253 | — | — | 94 | 0 | — | 0 | 91 | — | 74 | 0 |
| 254 | — | — | 100 | 100 | — | 0 | 95 | — | 99 | 98 |
| 255 | — | — | 99 | 0 | — | 0 | 95 | — | 86 | 91 |
| 256 | — | — | 100 | 100 | — | 100 | 97 | — | 100 | 99 |
| 257 | — | — | 100 | 100 | — | 99 | 95 | — | 100 | 100 |
| 258 | — | — | 99 | 0 | — | 42 | 87 | — | 97 | 78 |
| 259 | — | — | 99 | 16 | — | 0 | 63 | — | 82 | 93 |
| 260 | — | — | 100 | 95 | — | 0 | 92 | — | 94 | 99 |
| 261 | — | — | 99 | 100 | — | 100 | 95 | — | 100 | 100 |
| 262 | — | — | 99 | 99 | — | 69 | 96 | — | 100 | 100 |
| 263 | — | — | 99 | 100 | — | 100 | 95 | — | 100 | 100 |
| 264 | — | — | 99 | 88 | — | 0 | 95 | — | 98 | 99 |
| 265 | — | — | 97 | 0 | — | 60 | 86 | 32 | 61 | 0 |
| 266 | — | — | 100 | 100 | — | 97 | 94 | 98 | 99 | 99 |
| 267 | — | — | 98 | 0 | — | 0 | 91 | 9 | 86 | 97 |
| 268 | — | — | 99 | 100 | — | 98 | 87 | 63 | 99 | 75 |
| 269 | — | — | 100 | 0 | — | 87 | 83 | 9 | 9 | 0 |
| 270 | — | — | 100 | 100 | — | 98 | 86 | 0 | 99 | 96 |
| 271 | — | — | 99 | 9 | — | 0 | 82 | 0 | 83 | 0 |
| 272 | — | — | 64 | 0 | — | 0 | 91 | 17 | 55 | 0 |
| 273 | — | — | 63 | 0 | — | 0 | 13 | 46 | 41 | 0 |
| 274 | — | — | 100 | 100 | — | 51 | 94 | 48 | 99 | 77 |
| 275 | — | — | 100 | 82 | — | 0 | 85 | 9 | 74 | 0 |
| 276 | — | — | 100 | 100 | — | 0 | 92 | 9 | 98 | 100 |
| 277 | — | — | 99* | 0* | — | 0* | 89* | — | 88* | 94* |
| 278 | — | — | 99 | 100 | — | 100 | 95 | — | 100 | 100 |
| 279 | — | — | 100 | 100 | — | 100 | 93 | — | 100 | 100 |
| 280 | — | — | 100 | 100 | — | 100 | 95 | — | 100 | 100 |
| 281 | — | — | 100 | 100 | — | 97 | 96 | — | 100 | 100 |
| 282 | — | — | 99 | 99 | — | 0 | 93 | 65 | 99 | 100 |
| 283 | — | — | 100 | 98 | — | 0 | 93 | 0 | 93 | 99 |
| 284 | — | — | 100 | 99 | — | 92 | 95 | 6 | 99 | 100 |
| 285 | — | — | 99 | 85 | — | 83 | 94 | 98 | 100 | 100 |
| 286 | — | — | 99 | 96 | — | 79 | 95 | 64 | 99 | 100 |
| 287 | — | — | 100 | 100 | — | 99 | 95 | 17 | 100 | 99 |
| 288 | — | — | 100 | 91 | — | 73 | 94 | 32 | 96 | 99 |
| 289 | — | — | 100 | 96 | — | 0 | 94 | 11 | 94 | 97 |
| 290 | — | — | 95 | 0 | — | 0 | 85 | 11 | 19 | 0 |
| 291 | — | — | 99 | 80 | — | 0 | 94 | 6 | 98 | 94 |
| 292 | — | — | 99 | 98 | — | 69 | 93 | 11 | 99 | 99 |
| 293 | — | — | 99 | 91 | — | 0 | 95 | 6 | 99 | 100 |
| 294 | — | — | 99 | 100 | — | 98 | 96 | 11 | 98 | 97 |
| 295 | — | — | 99 | 0 | — | 0 | 94 | 0 | 80 | 0 |
| 296 | — | — | 100 | 96 | — | 86 | 96 | 17 | 99 | 97 |
| 297 | — | — | 99* | 100* | — | 99* | 94* | — | 99* | 100* |
| 298 | — | — | 100 | 100 | — | 0 | 96 | 0 | 98 | 0 |
| 299 | — | — | 99 | 99 | — | 90 | 97 | 0 | 99 | 99 |
| 300 | — | — | 99 | 98 | — | 0 | 91 | 48 | 97 | 99 |
| 301 | — | — | 100 | 94 | — | 0 | 95 | 6 | 91 | 96 |
| 302 | — | — | 100 | 100 | — | 0 | 94 | 17 | 98 | 100 |
| 303 | — | — | 100* | 99* | — | 98* | 96* | 0* | 74* | 81* |
| 304 | — | — | 0* | 0* | — | 0* | 27* | 0* | 0* | 0* |
| 305 | — | — | 63* | 0* | — | 0* | 23* | 11* | 0* | 0* |
| 306 | — | — | 99 | 96 | — | 0 | 94 | 6 | 91 | 99 |
| 307 | — | — | 100 | 99 | — | 0 | 97 | 11 | 97 | 99 |
| 308 | — | — | 99* | 100* | — | 0* | 88* | 11* | 41* | 0* |
| 309 | — | — | 100 | 100 | — | 100 | 97 | 11 | 100 | 100 |
| 310 | — | — | 99 | 100 | — | 99 | 96 | 61 | 100 | 100 |
| 311 | — | — | 98 | 9 | — | 0 | — | 100 | 99 | 95 |
| 312 | — | — | 99 | 82 | — | 0 | — | 9 | 99 | 92 |
| 313 | — | — | 99 | 100 | — | 0 | — | 95 | 100 | 99 |
| 314 | — | — | 99 | 100 | — | 100 | 93 | 32 | 100 | 100 |
| 315 | — | — | 99 | 100 | — | 100 | 95 | 86 | 100 | 100 |
| 316 | — | — | 99 | 99 | — | 95 | 93 | 6 | 99 | 100 |
| 317 | — | — | 100 | 100 | — | 100 | 95 | 17 | 99 | 100 |
| 318 | — | — | 100 | 16 | — | 0 | 91 | 0 | 9 | 87 |
| 319 | — | — | 99 | 24 | — | 0 | 98 | 6 | 28 | 81 |
| 320 | — | — | 99 | 67 | — | 0 | 100 | 0 | 28 | 99 |
| 321 | — | — | 100 | 100 | — | 100 | 100 | 83 | 100 | 100 |
| 322 | — | — | 62* | 0* | — | 0* | 0* | 63* | 28* | 0* |
| 323 | — | — | 100 | 99 | — | 0 | 100 | 70 | 100 | 99 |
| 324 | — | — | 99 | 99 | — | 82 | 93 | 9 | 97 | 99 |
| 325 | — | — | 94 | 91 | — | 51 | 96 | 0 | 97 | 99 |
| 326 | — | — | 100 | 100 | — | 51 | 96 | 67 | 98 | 100 |
| 327 | — | — | 99* | 98* | — | 0* | 94* | 0* | 86* | 0* |
| 328 | — | — | 100 | 98 | — | 0 | 95 | 9 | 94 | 98 |
| 329 | — | — | 99 | 99 | — | 0 | 94 | 0 | 90 | 99 |
| 330 | — | — | 100 | 100 | — | 0 | 96 | 9 | 98 | 99 |
| 331 | — | — | 100 | 100 | — | 99 | 95 | 85 | 100 | 100 |
| 332 | — | — | 100 | 99 | — | 60 | 95 | 9 | 96 | 99 |
| 333 | — | — | 100* | 100* | — | 99* | 91* | 0* | 100* | 100* |
| 334 | — | — | 100* | 100* | — | 90* | 93* | 0* | 100* | 100* |
| 335 | — | — | 100* | 100* | — | 82* | 91* | 0* | 99* | 99* |
| 336 | — | — | 99* | 88* | — | 99* | 96* | 0* | 74* | 87* |
| 337 | — | — | 19* | 0* | — | 0* | 23* | 0* | 0* | 0* |
| 338 | — | — | 100 | 100 | — | 80 | 93 | 0 | 99 | 99 |
| 339 | — | — | 100 | 99 | — | 78 | 95 | 0 | 96 | 96 |
| 340 | — | — | 99 | 98 | — | 0 | 92 | 0 | 94 | 95 |
| 341 | — | — | 91* | 24* | — | 0* | 0* | 9* | 0* | 0* |
| 342 | — | — | 100 | 99 | — | 0 | 95 | 0 | 98 | 99 |
| 343 | — | — | 100 | 97 | — | 64 | 94 | 0 | 93 | 98 |
| 344 | — | — | 100 | 0 | — | 0 | 95 | 17 | 89 | 99 |
| 345 | — | — | 64 | 0 | — | 0 | 27 | 0 | 0 | 0 |
| 346 | — | — | 99 | 0 | — | 0 | 38 | 9 | 9 | 0 |
| 347 | — | — | 93 | 0 | — | 0 | 70 | 26 | 9 | 0 |
| 348 | — | — | 0 | 0 | — | 0 | 8 | 0 | 0 | 27 |
| 349 | — | — | 100 | 99 | — | 99 | 88 | 0 | 99 | 95 |
| 350 | — | — | 99 | 100 | — | 82 | 91 | 0 | 99 | 100 |
| 351 | — | — | 100 | 100 | — | 99 | 93 | 54 | 100 | 100 |
| 352 | — | — | 100 | 100 | — | 87 | 92 | 18 | 97 | 77 |
| 353 | — | — | 0 | 0 | — | 0 | 8 | 0 | 41 | 0 |
| 354 | — | — | 100 | 99 | — | 0 | 93 | 0 | 98 | 97 |
| 355 | — | — | 0 | 0 | — | 0 | 3 | 0 | 28 | 0 |
| 356 | — | — | 98 | 57 | — | 0 | 72 | 17 | 0 | 0 |
| 357 | — | — | 63 | 0 | — | 100 | 50 | 0 | 55 | 0 |
| 358 | — | — | 99 | 0 | — | 0 | 90 | 0 | 41 | 0 |
| 359 | — | — | 98 | 0 | — | 0 | 35 | 0 | 80 | 0 |
| 360 | — | — | 100 | 99 | — | 0 | 95 | 9 | 99 | 98 |
| 361 | — | — | 99 | 0 | — | 0 | 93 | 9 | 74 | 80 |
| 362 | — | — | 100 | 99 | — | 0 | 86 | 0 | 83 | 49 |
| 363 | — | — | 99 | 0 | — | 0 | 53 | 9 | 0 | 0 |
| 364 | — | — | 100 | 100 | — | 0 | 58 | 0 | 98 | 83 |
| 365 | — | — | 100 | 100 | — | 40 | 91 | 54 | 99 | 96 |
| 366 | — | — | 100 | 100 | — | 64 | 91 | 41 | 99 | 97 |
| 367 | — | — | 99 | 100 | — | 89 | 90 | 9 | 98 | 97 |
| 368 | — | — | 99 | 100 | — | 0 | 92 | 0 | 99 | 96 |
| 369 | — | — | 99 | 100 | — | 98 | 92 | 74 | 99 | 99 |
| 370 | — | — | 99 | 100 | — | 87 | 89 | 100 | 100 | 100 |
| 371 | — | — | 31* | 0* | — | 0* | 48* | 0* | 0* | 0* |
| 372 | — | — | 99 | 0 | — | 0 | 91 | 0 | 74 | 0 |
| 373 | — | — | 99* | 98* | — | 60* | 92* | 0* | 95* | 99* |
| 374 | — | — | 99 | 99 | — | 0 | 94 | 0 | 90 | 98 |
| 375 | — | — | 99 | 26 | — | 0 | 92 | 32 | 68 | 62 |
| 376 | — | — | 90 | 0 | — | 0 | 78 | 0 | 54 | 67 |
| 377 | — | — | 0 | 0 | — | 0 | 3 | 0 | 27 | 33 |
| 378 | — | — | 100 | 0 | — | 0 | 93 | 0 | 74 | 75 |
| 379 | — | — | 99 | 0 | — | 0 | 86 | 0 | 27 | 0 |
| 380 | — | — | 100 | 91 | — | 0 | 91 | 0 | 99 | 99 |
| 381 | — | — | 94 | 0 | — | 0 | 78 | 0 | 0 | 0 |
| 382 | — | — | 100* | 99* | — | 0* | 96* | 0* | 95* | 97* |
| 383 | — | — | 100* | 99* | — | 35* | 96* | 0* | 91* | 98* |
| 384 | — | — | 100* | 99* | — | 0* | 95* | 0* | 67* | 95* |
| 385 | — | — | 99* | 99* | — | 0* | 95* | 0* | 95* | 99* |
| 386 | — | — | 99 | 100 | — | 0 | 96 | 0 | 98 | 95 |
| 387 | — | — | 0 | 0 | — | 0 | 0 | 0 | 0 | 0 |
| 388 | — | — | 99 | 100 | — | 60 | 99 | — | 100 | 100 |
| 389 | — | — | 98 | 100 | — | 0 | 91 | | 99 | 98 |
| 390 | — | — | 100 | 100 | — | 99 | 95 | 18 | 100 | 100 |
| 391 | — | — | 100 | 99 | — | 0 | 94 | 0 | 99 | 100 |
| 392 | — | — | 100 | 100 | — | 100 | 97 | 60 | 99 | 100 |
| 393 | — | — | 99 | 100 | — | 99 | 95 | 79 | 99 | 100 |
| 394 | — | — | 3* | 0* | — | 0* | 28* | 0* | 45* | 0* |
| 395 | — | — | 30 | 0 | — | 0 | 67 | 0 | 0 | 0 |
| 396 | — | — | 100* | 100* | — | 99* | 93* | 0* | 100* | 99* |
| 397 | — | — | 100* | 98* | — | 0* | 94* | 0* | 88* | 13* |
| 398 | — | — | 100 | 80 | — | 79 | 96 | 0 | 79 | 100 |
| 399 | — | — | 100 | 99 | — | 82 | 96 | 0 | 82 | 100 |
| 400 | — | — | 100 | 100 | — | 99 | 94 | 0 | 99 | 100 |
| 401 | — | — | 99 | 100 | — | 0 | 96 | 0 | 73 | 100 |
| 402 | — | — | 100 | 100 | — | 99 | 96 | 0 | 94 | 99 |
| 403 | — | — | 99 | 0 | — | 0 | 97 | 0 | 77 | 86 |
| 404 | — | — | 100 | 0 | — | 0 | 99 | 0 | 97 | 98 |
| 405 | — | — | 99 | 98 | — | 0 | 100 | 0 | 98 | 99 |
| 406 | — | — | 100 | 100 | — | 100 | 93 | 100 | 100 | 100 |
| 407 | — | — | 100 | 0 | — | 92 | 96 | 0 | 97 | 98 |
| 408 | — | — | 0* | 0* | — | 0* | 3* | 0* | 0* | 0* |
| 409 | — | — | 100 | 9 | — | 0 | 100 | 0 | 96 | 98 |
| 410 | — | — | 98 | 99 | — | 0 | 100 | 0 | 99 | 100 |
| 411 | — | — | 100 | 100 | — | 0 | 99 | 0 | 99 | 100 |
| 412 | — | — | 100* | 100* | — | 0* | 99* | 0* | 74* | 99* |
| 413 | — | — | 100* | 99* | — | 0* | 14* | 9* | 0* | 0* |
| 414 | — | — | 99* | 99* | — | 0* | 96* | 0* | 93* | 100* |
| 415 | — | — | 100* | 100* | — | 0* | 99* | 0* | 94* | 98* |
| 416 | — | — | 100* | 100* | — | 0* | 97* | 0* | 26* | 99* |
| 417 | — | — | 100* | 99* | — | 0* | 89* | 9* | 9* | 94* |
| 418 | — | — | 100 | 100 | — | 99 | 100 | 0 | 97 | 100 |
| 419 | — | — | 100 | 100 | — | 100 | 100 | 0 | 97 | 99 |
| 420 | — | — | 100 | 100 | — | 99 | 100 | 0 | 100 | 100 |
| 421 | — | — | 100 | 98 | — | 0 | 100 | 0 | 100 | 100 |
| 422 | — | — | 100 | 100 | — | 60 | 100 | 0 | 99 | 100 |
| 423 | — | — | 100* | 0* | — | 69* | 94* | 0* | 91* | 99* |
| 424 | — | — | 100 | 100 | — | 99 | 100 | 0 | 94 | 100 |
| 425 | — | — | 100 | 100 | — | 100 | 100 | 0 | 96 | 100 |
| 426 | — | — | 100 | 100 | — | 100 | 100 | 27 | 100 | 100 |
| 427 | — | — | 100 | 100 | — | 99 | 100 | 0 | 93 | 100 |
| 428 | — | — | 100 | 100 | — | 0 | 99 | 0 | 100 | 100 |
| 429 | — | — | 99 | 77 | — | 0 | 99 | 0 | 98 | 98 |
| 430 | — | — | 100 | 100 | — | 100 | 100 | 0 | 100 | 100 |
| 431 | — | — | 9 | 9 | — | 0 | 98 | 9 | 0 | 0 |
| 432 | — | — | 33 | 0 | — | 0 | 25 | 0 | 0 | 0 |
| 433 | — | — | 50 | 0 | — | 0 | 92 | 0 | 0 | 0 |
| 434 | — | — | 96 | 9 | — | 0 | 99 | 0 | 68 | 76 |
| 435 | — | — | 99 | 24 | — | 0 | 99 | 0 | 68 | 0 |
| 436 | — | — | 87 | 98 | — | 0 | 81 | 0 | 0 | 0 |
| 437 | — | — | 99 | 80 | — | 60 | 100 | 0 | 98 | 98 |
| 438 | — | — | 0 | 0 | — | 0 | 86* | 0 | 0 | 0 |
| 439 | — | — | 97 | 100 | — | 95 | 94 | — | 100 | 100 |
| 440 | — | — | 99 | 100 | — | 98 | 95 | — | 98 | 99 |
| 441 | — | — | 97 | 99 | — | 0 | 97 | — | 97 | 99 |
| 442 | — | — | 99 | 99 | — | 0 | 95 | — | 99 | 100 |
| 443 | — | — | 99 | 99 | — | 99 | 97 | — | 89 | 100 |
| 444 | — | — | 99 | 100 | — | 100 | 98 | — | 95 | 99 |
| 445 | — | — | 97* | 100* | — | 92* | 95 | — | 97* | 98* |
| 446 | — | — | 96* | 100* | — | 0* | 95* | — | 68* | 91* |
| 447 | — | — | 94 | 80 | — | 0 | 30 | — | 0 | 81 |
| 448 | — | — | 97 | 100 | — | 89 | 98 | — | 95 | 98 |
| 449 | — | — | 99 | 100 | — | 99 | 97 | — | 99 | 100 |
| 450 | — | — | 97 | 100 | — | 99 | 97 | — | 100 | 100 |
| 451 | — | — | 0 | 0 | — | 0 | 82 | — | 19 | 0 |
| 452 | — | — | 55 | 0 | — | 0 | 86 | — | 86 | 76 |
| 453 | — | — | 99 | 100 | — | 90* | 93 | — | 100 | 100 |
| 454 | — | — | 77 | 100 | — | 33 | 93 | — | 100 | 100 |
| 455 | — | — | 99 | 95 | — | 0 | 95 | — | 99 | 68 |
| 456 | — | — | 0 | 0 | — | 0 | 88 | — | 0 | 43 |
| 457 | — | — | 85 | 0 | — | 0 | 91 | — | 97 | 97 |
| 458 | — | — | 96 | 16 | — | 0 | 92 | — | 95 | 89 |
| 459 | — | — | 99* | 63* | — | 0* | 95* | 9* | 98* | 98* |
| 460 | — | — | 99* | 85* | — | 0* | 97* | 0* | 93* | 96* |
| 461 | — | — | 99* | 99* | — | 0* | 94* | 0* | 97* | 98* |
| 462 | — | — | 100* | 100* | — | 0* | 96* | 0* | 78* | 99* |
| 463 | — | — | 99* | 99* | — | 0* | 95* | 0* | 100* | 99* |
| 464 | — | — | 99* | 89* | — | 0* | 72* | 0* | 99* | 100* |
| 465 | — | — | 100* | 99* | — | 0* | 88* | 0* | 99* | 99* |
| 466 | — | — | 99* | 100* | — | 0* | 82* | 0* | 93* | 100* |
| 467 | — | — | 94* | 0* | — | 0* | 32* | 0* | 71* | 96* |
| 468 | — | — | 99* | 99* | — | 0* | 96* | 0* | 91* | 58* |
| 469 | — | — | 65* | 0* | — | 0* | 79* | 0* | 81* | 0* |
| 470 | — | — | 99* | 99* | — | 0* | 92* | 0* | 99* | 87* |
| 471 | — | — | 100 | 100 | — | 100 | 97 | 0 | 98 | 100 |
| 472 | — | — | 100 | 100 | — | 100 | 96 | 80 | 100 | 100 |
| 473 | — | — | 99 | 100 | — | 0 | 97 | 0 | 100 | 100 |
| 474 | — | — | 99 | 57 | — | 0 | 90 | 0 | 36 | 95 |
| 475 | — | — | 98 | 26 | — | 0 | 92 | 0 | 87 | 42 |
| 476 | — | — | 0 | 0 | — | 0 | 59 | 0 | 78 | 47 |
| 477 | — | — | 9 | 0 | — | 0 | 0 | 0 | 75 | 0 |
| 478 | — | — | 100 | 93 | — | 0 | 93 | 0 | 96 | 97 |
| 479 | — | — | 100 | 100 | — | 95 | 94 | 0 | 99 | 100 |
| 480 | — | — | 100 | 100 | — | 95 | 96 | 0 | 99 | 100 |
| 481 | — | — | 100 | 100 | — | 99 | 95 | 0 | 97 | 100 |
| 482 | — | — | 99 | 99 | — | 69 | 95 | 0 | 91 | 99 |
| 483 | — | — | 100 | 99 | — | 0 | 94 | 32 | 98 | 100 |
| 484 | — | — | 100 | 100 | — | 89 | 95 | 28 | 100 | 100 |
| 485 | — | — | 96 | 88 | — | 0 | 95 | 0 | 98 | 98 |
| 486 | — | — | 66 | 0 | — | 0 | 0 | 0 | 14 | 0 |
| 487 | — | — | 96 | 0 | — | 0 | 88 | 0 | 78 | 0 |
| 488 | — | — | 99 | 100 | — | 100 | 93 | 41 | 100 | 100 |
| 489 | — | — | 96* | 98* | — | 0* | 93* | 0* | 100* | 100* |
| 490 | — | — | 97* | 99* | — | 0* | 87* | 0* | 76* | 100* |
| 491 | — | — | 92* | 99* | — | 0* | 91* | 0* | 0* | 0* |
| 492 | — | — | 94* | 100* | — | 0* | 82* | 0* | 0* | 0* |
| 493 | — | — | 96* | 99* | — | 0* | 93* | 0* | 97* | 99* |
| 494 | — | — | 89* | 99* | — | 0* | 89* | 0* | 99* | 99* |
| 495 | — | — | 97* | 99* | — | 0* | 31* | 0* | 99* | 100* |
| 496 | — | — | 93* | 99* | — | 0* | 6* | 0* | 0* | 97* |
| 497 | — | — | 97 | 99 | — | 100 | 97 | 0 | 100 | 100 |
| 498 | — | — | 99 | 100 | — | 95 | 95 | 0 | 100 | 100 |
| 499 | — | — | 99 | 100 | — | 100 | 93 | 100 | 100 | 100 |
| 500 | — | — | 98 | 100 | — | 100 | 94 | 98 | 100 | 100 |
| 501 | — | — | 98 | 99 | — | 98 | 95 | 0 | 67 | 99 |
| 502 | — | — | 90* | 92* | — | 60* | 93* | 0* | 98* | 100* |
| 503 | — | — | 99 | 99 | — | 99 | 96 | 0 | 100 | 100 |
| 504 | — | — | 99 | 100 | — | 100 | 97 | 0 | 99 | 100 |
| 505 | — | — | 99 | 100 | — | 98 | 97 | 78 | 100 | 100 |
| 506 | — | — | 100 | 100 | — | 92 | 99 | 78 | 100 | 99 |
| 507 | — | — | 100 | 95 | — | 0 | 88 | 0 | 100 | 98 |
| 508 | — | — | 100 | 100 | — | 0 | 94 | 78 | 99 | 99 |
| 509 | — | — | 98 | 77 | — | 0 | 92 | 9 | 85 | 78 |
| 510 | — | — | 99 | 100 | — | 100 | 99 | 9 | 98 | 100 |
| 511 | — | — | 99 | 99 | — | 99 | 99 | 0 | 99 | 100 |
| 512 | — | — | 97 | 98 | — | 0 | 98 | 40 | 79 | 99 |
| 513 | — | — | 99 | 100 | — | 100 | 100 | 100 | 100 | 100 |
| 514 | — | — | 99 | 99 | — | 73 | 100 | 0 | 93 | 99 |
| 515 | — | — | 97 | 99 | — | 0 | 100 | 100 | 100 | 100 |
| 516 | — | — | 97 | 100 | — | 100 | 100 | 100 | 100 | 100 |
| 517 | — | — | 100 | 100 | — | 96 | 98 | 0 | 99 | 100 |
| 518 | — | — | 100 | 99 | — | 99 | 98 | 0 | 98 | 100 |
| 519 | — | — | 100 | 100 | — | 100 | 97 | 0 | 100 | 100 |
| 520 | — | — | 100 | 100 | — | 0 | 97 | 0 | 99 | 100 |
| 521 | — | — | 99* | 99* | — | 0* | 98* | 0* | 98* | 90* |
| 522 | — | — | 100* | 100* | — | 0* | 98* | 0* | 99* | 96* |
| 523 | — | — | 100* | 100* | — | 0* | 98* | 0* | 99* | 97* |
| 524 | — | — | 99* | 100* | — | 60* | 98* | 85* | 99* | 97 |
| 525 | — | — | 100* | 100* | — | 80* | 98* | 68* | 100* | 98* |
| 526 | — | — | 100 | 100 | — | 100 | 99 | 0 | 100 | 100 |
| 527 | — | — | 100 | 99 | — | 0 | 95 | 37 | 91 | 88 |
| 528 | — | — | 100 | 100 | — | 99 | 99 | 15 | 96 | 100 |
| 529 | — | — | 0 | 0 | — | 0 | 82 | 9 | 48 | 0 |
| 530 | — | — | 0 | 0 | — | 0 | 58 | 0 | 88 | 71 |
| 531 | — | — | 100 | 0 | — | 0 | 99 | 41 | 99 | 95 |
| 532 | — | — | 97 | 0 | — | 0 | 99 | 0 | 99 | 91 |
| 533 | — | — | 0 | 0 | — | 0 | 49 | 15 | 92 | 82 |
| 534 | — | — | 38 | 0 | — | 0 | 2 | 0 | 0 | 0 |
| 535 | — | — | 0 | 0 | — | 0 | 2 | 0 | 0 | 0 |
| 536 | — | — | 89 | 68 | — | 0 | 90 | 37 | 98 | 98 |
| 537 | — | — | 99 | 76 | — | 60 | 97 | 9 | 100 | 99 |
| 538 | — | — | 99 | 0 | — | 0 | 99 | 27 | 100 | 100 |
| 539 | — | — | 36 | 0 | — | 0 | 7 | 0 | 93 | 95 |
| 540 | — | — | 15 | 0 | — | 0 | 3 | 0 | 0 | 21 |
| 541 | — | — | 0 | 0 | — | 0 | 0 | 0 | 0 | 0 |
| 542 | — | — | 99* | 96* | — | 0* | 97* | 0* | 98* | 89* |
| 543 | — | — | 99* | 60* | — | 0* | 97* | 0* | 95* | 0* |
| 544 | — | — | 84 | 98 | — | 0 | 82 | 9 | 91 | 86 |
| 545 | — | — | 100 | 100 | — | 98 | 99 | 27 | 100 | 99 |
| 546 | — | — | 100 | 100 | — | 99 | 98 | 0 | 100 | 100 |
| 547 | — | — | 99 | 88 | — | 60 | 97 | 0 | 99 | 100 |
| 548 | — | — | 98 | 100 | — | 94 | 96 | 0 | 99 | 100 |
| 549 | — | — | 100 | 100 | — | 99 | 98 | 0 | 100 | 100 |
| 550 | — | — | 100 | 100 | — | 100 | 97 | 0 | 99 | 100 |
| 551 | — | — | 100 | 100 | — | 96 | 98 | 0 | 100 | 100 |
| 552 | — | — | 99 | 100 | — | 0 | 96 | 0 | 46 | 98 |
| 553 | — | — | 100 | 100 | — | 100 | 99 | 100 | 100 | 100 |
| 554 | — | — | 100 | 100 | — | 100 | 99 | 100 | 100 | 100 |
| 555 | — | — | 100 | 99 | — | 69 | 98 | 0 | 100 | 98 |
| 556 | — | — | 100 | 99 | — | 98 | 95 | 87 | 100 | 97 |
| 557 | — | — | 100 | 99 | — | 78 | 96 | 43 | 99 | 87 |
| 558 | — | — | 100 | 0 | — | 0 | 94 | 15 | 99 | 100 |
| 559 | — | — | 94 | 41 | — | 0 | 95 | 29 | 88 | 98 |
| 560 | — | — | 99* | 0* | — | 0* | 65* | 33* | 79* | 93* |
| 561 | — | — | 99 | 100 | — | 97 | 100 | 9 | 100 | 100 |
| 562 | — | — | 100 | 100 | — | 99 | 100 | 0 | 99 | 100 |
| 563 | — | — | 100 | 100 | — | 100 | 100 | 0 | 94 | 100 |
| 564 | — | — | 99* | 100* | — | 64* | 99* | 66* | 100* | 100* |
| 565 | — | — | 100 | 100 | — | 100 | 96 | 97 | 100 | 100 |
| 566 | — | — | 100 | 100 | — | 100 | 97 | 97 | 100 | 100 |
| 567 | — | — | 99 | 67 | — | 0 | 94 | 7 | 99 | 100 |
| 568 | — | — | 99 | 100 | — | 98 | 94 | 72 | 100 | 100 |
| 569 | — | — | 100 | 100 | — | 100 | 97 | 84 | 100 | 99 |
| 570 | — | — | 0 | 0 | — | 0 | 97 | 0 | 13 | 69 |
| 571 | — | — | 33 | 0 | — | 0 | 82 | 0 | 90 | 0 |
| 572 | — | — | 100* | 99* | — | 92* | 95* | 0* | 99* | 71* |
| 573 | — | — | 100 | 100 | — | 0 | 96 | 22 | 100 | 99 |
| 574 | — | — | 100 | 0 | — | 60 | 100 | 0 | 98 | 97 |
| 575 | — | — | 96 | 0 | — | 0 | 73 | 0 | 76 | 72 |
| 576 | — | — | 100 | 99 | — | 100 | 100 | 100 | 100 | 99 |
| 577 | — | — | 100 | 100 | — | 87 | 100 | 97 | 100 | 100 |
| 578 | 0 | 58 | 98 | 37 | 0 | 0 | 100 | — | 74 | 98 |
| 579 | 0 | 92 | 99 | 97 | 0 | 0 | 100 | — | 93 | 100 |
| 580 | 0 | 88 | 98 | 0 | 0 | 0 | 100 | — | 84 | 90 |
| 581 | — | — | 99* | 33* | — | 0* | 96* | 84* | 98* | 21* |
| 582 | — | — | 100 | 100 | — | 99 | 97 | 15 | 99 | 100 |
| 583 | — | — | 99 | 97 | — | 0 | 94 | 0 | 85 | 83 |
| 584 | — | — | 99 | 99 | — | 0 | 98 | 0 | 100 | 99 |
| 585 | — | — | 100 | 99 | — | 0 | 100 | 0 | 100 | 99 |
| 586 | — | — | 0 | 0 | — | 0 | 3 | 0 | 0 | 21 |
| 587 | — | — | 25 | 80 | — | 0 | 97 | 7 | 0 | 97 |
| 588 | — | — | 100 | 100 | — | 100 | 97 | 100 | 100 | 100 |
| 589 | — | — | 100 | 99 | — | 98 | 98 | 97 | 100 | 100 |
| 590 | — | — | 100* | 100* | — | 94* | 96* | 7* | 100* | 100* |
| 591 | — | — | 100 | 100 | — | 0 | 97 | 96 | 98 | 98 |
| 592 | — | — | 100 | 100 | — | 100 | 96 | 100 | 100 | 100 |
| 593 | — | — | 100 | 100 | — | 99 | 96 | 99 | 100 | 100 |
| 594 | — | — | 100 | 99 | — | 0 | 94 | 78 | 99 | 100 |
| 595 | — | — | 100 | 80 | — | 0 | 96 | 22 | 74 | 98 |
| 596 | — | — | 94 | 73 | — | 0 | 98 | 0 | 74 | 97 |
| 597 | — | — | 100 | 100 | — | 97 | 98 | 99 | | 100 |
| 598 | — | — | 100 | 100 | — | 100 | 99 | 99 | 100 | 100 |
| 599 | — | — | 26 | 0 | — | 0 | 9 | 0 | 0 | 0 |
| 600 | — | — | 86 | 100 | — | 0 | 94 | 7 | 0 | 99 |
| 601 | — | — | 99* | 98* | — | 87* | 99* | 7* | 97* | 97* |
| 602 | — | — | 99* | 91* | — | 0* | 99* | 0* | 98* | 81* |
| 603 | — | — | 99 | 100 | — | 99 | 99 | 98 | 100 | 100 |
| 604 | — | — | 99 | 100 | — | 100 | 99 | 97 | 100 | 100 |
| 605 | — | — | 19 | 0 | — | 0 | 73 | 0 | 0 | 94 |
| 606 | — | — | 99 | 100 | — | 0 | 99 | 0 | 28 | 100 |
| 607 | — | — | 100* | 96* | — | 31* | 99* | 7* | 86* | 82* |
| 608 | — | — | 17* | 0* | — | 0* | 4* | 7* | 41* | 0* |
| 609 | — | — | 99 | 100 | — | 100 | 99 | 100 | 100 | 100 |
| 610 | — | — | 99 | 100 | — | 99 | 99 | 100 | 100 | 100 |
| 611 | — | — | 91 | 66 | — | 0 | 81 | 0 | 16 | 57 |
| 612 | — | — | 99 | 100 | — | 60 | 96 | 0 | 8 | 100 |
| 613 | — | — | 99* | 100* | — | 64* | 99* | 15* | 97* | 21* |
| 614 | — | — | 89* | 0* | — | 0* | 92* | 0* | 28* | 0* |
| 615 | — | — | 100 | 100 | — | 0 | 100 | 0 | 99 | 100 |
| 616 | — | — | 100 | 0 | — | 0 | 99 | 0 | 98 | 99 |
| 617 | — | — | 99 | 99 | — | 0 | 100 | 100 | 100 | 99 |
| 618 | — | — | 98 | 85 | — | 0 | 98 | 99 | 99 | 97 |
| 619 | — | — | 99 | 53 | — | 0 | 96 | 51 | 98 | 99 |
| 620 | — | — | 100 | 99 | — | 90 | 98 | 100 | 100 | 99 |
| 621 | — | — | 100 | 100 | — | 99 | 98 | 100 | 100 | 100 |
| 622 | — | — | 100 | 100 | — | 99 | 99 | 100 | 100 | 100 |
| 623 | — | — | 98 | 33 | — | 0 | 97 | 0 | 98 | 100 |
| 624 | — | — | 99 | 64 | — | 0 | 94 | 0 | 99 | 90 |
| 625 | — | — | 98 | 0 | — | 0 | 97 | 0 | 0 | 94 |
| 626 | — | — | 99 | 99 | — | 0 | 100 | 17 | 79 | 97 |
| 627 | — | — | 99 | 99 | — | 0 | 99 | 40 | 95 | 98 |
| 628 | — | — | 99 | 100 | — | 0 | 100 | 0 | 99 | 99 |
| 629 | 0 | 0 | 0 | 0 | 0 | 0 | 0 | — | 0 | 0 |
| 630 | 0 | 0 | 0 | 0 | 0 | 69 | 0 | — | 95 | 0 |
| 631 | — | — | 99 | 99 | — | 0 | 100 | 40 | 99 | 100 |
| 632 | — | — | 99 | 100 | — | 0 | 98 | 99 | 100 | 97 |
| 633 | — | — | 0* | 0* | — | 0* | 0* | 0* | 44* | 0* |
| 634 | — | — | 0* | 0* | — | 0* | 0* | 0* | 0* | 0* |
| 635 | — | — | 99* | 0* | — | 0* | 81* | 0* | 39* | 0* |
| 636 | — | — | 90* | 24* | — | 0* | 95* | 0* | 52* | 0* |
| 637 | — | — | 97* | 0* | — | 0* | 92* | 0* | 65* | 0* |
| 638 | — | — | 99 | 99 | — | 55 | 99 | 0 | 95 | 56 |
| 639 | 0 | 0 | 0 | 0 | 0 | 0 | 0 | — | 0 | 0 |
| 640 | 0 | 0 | 0 | 0 | 0 | 0 | 0 | — | 0 | 0 |
| 641 | — | — | 95 | 96 | — | 94 | 99 | 100 | 100 | 83 |
| 642 | — | — | 97* | 0* | — | 0* | 94* | 0* | 98* | 56* |
| 643 | — | — | 0* | 0* | — | 0* | 87* | 0* | 52* | 0* |
| 644 | 0 | — | 94 | 0 | 0 | 0 | 0 | — | 0 | 0 |
| 645 | — | — | 99* | 17* | — | 0* | 95* | 0* | 83* | 35* |
| 646 | — | — | 93* | 47* | — | 0* | 97* | 0* | 92* | 35* |
| 647 | — | — | 0* | 0* | — | 0* | 95 | 0* | 0* | 69* |
| 648 | 9 | 0 | 0 | 0 | 0 | 0 | 0 | — | 0 | 0 |
| 649 | — | — | 0* | 0* | — | 0* | 97* | 0* | 61* | 0* |
| 650 | — | — | 0* | 0* | — | 0* | 0* | 9* | 0* | 0* |
| 651 | — | — | 97 | 0 | — | 60 | 97 | 0 | 95 | 79 |
| 652 | — | — | 0* | 0* | — | 0* | 0* | 6* | 0* | 0* |
| 653 | — | — | 99* | 99* | — | 78* | 100* | 99* | 100* | 99* |
| 654 | — | — | 100* | 99* | — | 91* | 98* | 100* | 99* | 100* |
| 655 | — | — | 8* | 0* | — | 0* | 97* | 49* | 9* | 0* |
| 656 | — | — | 100 | 100 | — | 20 | 99 | 9 | 100 | 100 |
| 657 | — | — | 99* | 99* | — | 99* | 99* | 100* | 100* | 96* |
| 658 | — | — | 0 | 0 | — | 0 | 55 | 0 | 61 | 61 |
| 659 | — | — | — | — | — | — | — | — | — | — |
| 660 | — | — | 100 | 98 | — | 82 | 100 | 9 | 92 | 100 |
| 661 | — | — | 99 | 99 | — | 87 | 99 | 0 | 100 | 100 |
| 662 | — | — | 100 | 99 | — | 100 | 99 | 0 | 98 | 100 |
| 663 | — | — | 100 | 99 | — | 99 | 99 | 54 | 99 | 100 |
| 664 | — | — | 100 | 99 | — | 96 | 99 | 46 | 97 | 100 |
| 665 | — | — | 100 | 99 | — | 99 | 99 | 54 | 99 | 100 |
| 666 | — | — | 100 | 100 | — | 99 | 99 | 100 | 100 | 100 |
| 667 | — | — | 100 | 100 | — | 100 | 99 | 8 | 97 | 76 |
| 668 | — | — | 100 | 99 | — | 91 | 99 | 0 | 85 | 96 |
| 669 | — | — | 100 | 100 | — | 99 | 99 | 73 | 98 | 0 |
| 670 | — | — | 100 | 100 | — | 100 | 98 | 0 | 98 | 94 |
| 671 | — | — | 99 | 100 | — | 92 | 95 | 17 | 78 | 0 |
| 672 | — | — | 96 | 0 | — | 0 | 87 | 0 | 0 | 0 |
| 673 | — | — | 99 | 100 | — | 99 | 99 | 9 | 100 | 90 |
| 674 | — | — | 99 | 93 | — | 98 | 99 | 98 | 100 | 94 |
| 675 | — | — | 100 | 100 | — | 99 | 98 | 100 | 100 | 100 |
| 676 | — | — | 100 | 99 | — | 99 | 99 | 99 | 100 | 98 |
| 677 | — | — | 100 | 100 | — | 100 | 99 | 100 | 100 | 100 |
| 678 | — | — | 100 | 100 | — | 100 | 99 | 100 | 100 | 100 |
| 679 | — | — | 100 | 100 | — | 99 | 99 | 18 | 100 | 100 |
| 680 | — | — | 99 | 100 | — | 98 | 100 | 0 | 100 | 100 |
| 681 | — | — | 99 | 100 | — | 98 | 99 | 0 | 99 | 100 |
| 682 | — | — | 100 | 100 | — | 99 | 100 | 97 | 100 | 100 |
| 683 | — | — | 100* | 97* | — | 0* | 67* | 8* | 0* | 98* |
| 684 | — | — | 100 | 100 | — | 100 | 99 | 53 | 99 | 100 |
| 685 | — | — | 100* | 99* | — | 0* | 87* | 0* | 68* | 64* |
| 686 | — | — | 100* | 100* | — | 82* | 98* | 98* | 99* | 98* |
| 687 | — | — | 100* | 100* | — | 99* | 98* | 100* | 100* | 100* |
| 688 | — | — | 100* | 33* | — | 0* | 97* | 8* | 0* | 78* |
| 689 | — | — | 0 | 0 | — | 0 | 0 | 0 | 0 | 0 |
| 690 | — | — | 78 | 0 | — | 0 | 98 | 0 | 74 | 81 |
| 691 | — | — | 99* | 99* | — | 51* | 95* | 39* | 82* | 64* |
| 692 | — | — | 100 | 70 | — | 0 | 99 | 8 | 0 | 79 |
| 693 | — | — | 100 | 100 | — | 99 | 99 | 100 | 100 | 100 |
| 694 | — | — | 100* | 100* | — | 99* | 98* | 0* | 99* | 98* |
| 695 | — | — | 100 | 99 | — | 97 | 99 | 97 | 99 | 96 |
| 696 | — | — | 100* | 100* | — | 100* | 99* | 99* | 99* | 100* |
| 697 | — | — | 100 | 91 | — | 0 | 98 | 8 | 19 | 93 |
| 698 | — | — | 99 | 0 | — | 0 | 99 | 0 | 83 | 56 |
| 699 | — | — | 100 | 99 | — | 0 | 99 | 0 | 41 | 99 |
| 700 | — | — | 93* | 0* | — | 0* | 53* | 0* | 0* | 0* |
| 701 | — | — | 100 | 100 | — | 91 | 99 | 7 | 100 | 95 |
| 702 | — | — | 100 | 100 | — | 99 | 99 | 59 | 86 | 99 |
| 703 | — | — | 100 | 100 | — | 100 | 99 | 39 | 98 | 97 |
| 704 | — | — | 64 | 80 | — | 0 | 0 | 0 | 9 | 0 |
| 705 | — | — | 99 | 100 | — | 99 | 99 | 100 | 100 | 100 |
| 706 | — | — | 57 | 100 | — | 0 | 99 | 25 | 86 | 100 |
| 707 | — | — | 100 | 100 | — | 100 | 99 | 67 | 100 | 100 |
| 708 | — | — | 100 | 0 | — | 82 | 99 | 7 | 80 | 89 |
| 709 | — | — | 100 | 100 | — | 100 | 99 | 100 | 100 | 100 |
| 710 | — | — | 100 | 100 | — | 100 | 98 | 99 | 100 | 98 |
| 711 | — | — | 100 | 100 | — | 99 | 99 | 98 | 99 | 99 |
| 712 | — | — | 100* | 57* | — | 0* | 95 | 7* | 86* | 61* |
| 713 | — | — | 100 | 100 | — | 100 | 100 | 78 | 100 | 100 |
| 714 | — | — | 100 | 100 | — | 100 | 99 | 100 | 100 | 100 |
| 715 | — | — | 100 | 100 | — | 78 | 99 | 100 | 99 | 73 |
| 716 | — | — | 100 | 100 | — | 95 | 100 | 100 | 99 | 99 |
| 717 | — | — | 100 | 100 | — | 0 | 99 | 0 | 96 | 79 |
| 718 | — | — | 100 | 100 | — | 97 | 100 | 99 | 100 | 100 |
| 719 | — | — | 99 | 100 | — | 98 | 99 | 99 | 100 | 100 |
| 720 | 0 | 0 | 0 | 0 | 0 | 0 | 0 | — | 0 | 94 |
| 721 | 0 | 0 | 0 | 0 | 0 | 0 | 0 | — | 0 | 0 |
| 722 | 9 | 0 | 0 | 0 | 0 | 0 | 0 | — | 0 | 0 |
| 723 | — | — | 100 | 99 | — | 0 | 97 | 66 | 68 | 95 |
| 724 | — | — | 100 | 100 | — | 97 | 98 | 0 | 100 | 99 |
| 725 | — | — | 100 | 99 | — | 73 | 99 | 15 | 91 | 98 |
| 726 | — | — | 99 | 99 | — | 0 | 99 | 0 | 80 | 89 |
| 727 | — | — | 98 | 0 | — | 0 | 89 | 15 | 74 | 21 |
| 728 | — | — | 100 | 100 | — | 100 | 99 | 100 | 100 | 100 |
| 729 | — | — | 100 | 100 | — | 99 | 99 | — | 100 | 100 |
| 730 | — | — | 100 | 100 | — | 99 | 100 | 9 | 99 | 100 |
| 731 | — | — | 99 | 100 | — | 99 | 100 | 0 | 99 | 100 |
| 732 | — | — | 100 | 100 | — | 100 | 99 | 0 | 99 | 100 |
| 733 | — | — | 100 | 100 | — | 100 | 100 | 41 | 100 | 100 |
| 734 | — | — | 100 | 99 | — | 99 | 99 | 41 | 100 | 95 |
| 735 | — | — | 100 | 100 | — | 99 | 100 | 9 | 100 | 100 |
| 736 | — | — | 99 | 100 | — | 97 | 100 | 99 | 100 | 100 |
| 737 | — | — | 100 | 100 | — | 100 | 100 | 100 | 100 | 100 |
| 738 | — | — | 100 | 100 | — | 100 | 100 | 96 | 100 | 100 |
| 739 | — | — | 100 | 100 | — | 100 | 100 | 100 | 100 | 100 |
| 740 | 37 | 99 | 100 | 100 | 0 | 100 | 100 | — | 100 | 100 |
| 741 | — | — | 100 | 99 | — | 92 | — | 0 | 99 | 72 |

What is claimed is:

1. A compound selected from Formula 1, N-oxides, and salts thereof, 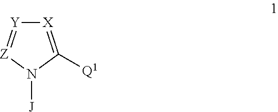
J is Q² or R¹;
X is N, CR² or CQ³;
Y is N or CR³;
Z is N or CR⁴;
Q¹ is a phenyl ring or a naphthalenyl ring system, each ring or ring system optionally substituted with up to 5 substituents independently selected from R^{5a}; or a 5- to 6-membered fully unsaturated heterocyclic ring or an 8- to 10-membered heteroaromatic bicyclic ring system, each ring or ring system containing ring members selected from carbon atoms and up to 4 heteroatoms independently selected from up to 2O, up to 2S and up to 4N atoms, wherein up to 3 carbon atom ring members are independently selected from C(═O) and C(═S), and the sulfur atom ring members are independently selected from S(═O)ₚ(═NR⁶)_{f}, each ring or ring system optionally substituted with up to 5 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W¹;
W¹ is a phenyl ring optionally substituted with up to 5 substituents independently selected from R^{5a}; or a 5- to 6-membered fully unsaturated heterocyclic ring containing ring members selected from carbon atoms and up to 4 heteroatoms independently selected from up to 2O, up to 2S and up to 4N atoms, wherein up to 2 carbon atom ring members are independently selected from C(═O) and C(═S), and the sulfur atom ring members are independently selected from S(═O)_{P}(═NR⁶)_{f}, the ring optionally substituted with up to 5 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members;
Q² is a phenyl ring or a naphthalenyl ring system, each ring or ring system optionally substituted with up to 5 substituents independently selected from R^{5b}; or a 5- to 6-membered fully unsaturated heterocyclic ring or an 8- to 10-membered heteroaromatic bicyclic ring system, each ring or ring system containing ring members selected from carbon atoms and up to 4 heteroatoms independently selected from up to 2O, up to 2S and up to 4N atoms, wherein up to 3 carbon atom ring members are independently selected from C(═O) and C(═S), and the sulfur atom ring members are independently selected from S(═O)ₚ(═NR⁶)_{f}, each ring or ring system optionally substituted with up to 5 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W²;
W² is a phenyl ring optionally substituted with up to 5 substituents independently selected from R^{5b}; or a 5- to 6-membered fully unsaturated heterocyclic ring containing ring members selected from carbon atoms and up to 4 heteroatoms independently selected from up to 2O, up to 2S and up to 4N atoms, wherein up to 2 carbon atom ring members are independently selected from C(═O) and C(═S), and the sulfur atom ring members are independently selected from S(═O)_{P}(═NR⁶)_{f}, the ring optionally substituted with up to 5 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members;
Q³ is a phenyl ring or a naphthalenyl ring system, each ring or ring system optionally substituted with up to 5 substituents independently selected from R^{5c}; or a 5- to 6-membered fully unsaturated heterocyclic ring or an 8- to 10-membered heteroaromatic bicyclic ring system, each ring or ring system containing ring members selected from carbon atoms and up to 4 heteroatoms independently selected from up to 2O, up to 2S and up to 4N atoms, wherein up to 3 carbon atom ring members are independently selected from C(═O) and C(═S), and the sulfur atom ring members are independently selected from S(═O)ₚ(═NR⁶)_{f}, each ring or ring system optionally substituted with up to 5 substituents independently selected from R^{5c} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W³;
W³ is a phenyl ring optionally substituted with up to 5 substituents independently selected from R^{5c}; or a 5- to 6-membered fully unsaturated heterocyclic ring containing ring members selected from carbon atoms and up to 4 heteroatoms independently selected from up to 2O, up to 2S and up to 4N atoms, wherein up to 2 carbon atom ring members are independently selected from C(═O) and C(═S), and the sulfur atom ring members are independently selected from S(═O)ₚ(═NR⁶)_{f}, the ring optionally substituted with up to 5 substituents independently selected from R^{5c} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members;
R¹ is C₁-C₇ alkyl, C₂-C₇ alkenyl, C₂-C₇ alkynyl, C₁-C₇ haloalkyl, C₂-C₇ haloalkenyl, C₃-C₇ cycloalkyl, C₃-C₇ halocycloalkyl, C₄-C₁₀ alkylcycloalkyl, C₄-C₁₀ cycloalkylalkyl, C₆-C₁₄ cycloalkylcycloalkyl, C₁-C₇ alkoxy, C₁-C₇ haloalkoxy, C₂-C₇ alkoxyalkyl, C₁-C₇ alkylthio, C₁-C₇ haloalkylthio, C₂-C₇ alkylthioalkyl, C₁-C₇ alkylsulfinyl, C₁-C₇ alkylsulfonyl, C₁-C₇ haloalkylsulfinyl, C₁-C₇ haloalkylsulfonyl, C₁-C₇ alkylamino, C₂-C₇ dialkylamino, C₂-C₇ alkylcarbonylamino or C₁-C₇ hydroxyalkyl;
each R², R³ and R⁴ is independently H, halogen, cyano, amino, nitro, —CHO, C₂-C₇ alkenyl, C₂-C₇ alkynyl, C₂-C₇ haloalkenyl, C₃-C₇ cycloalkyl, C₃-C₇ halocycloalkyl, C₄-C₁₀ alkylcycloalkyl, C₄-C₁₀ cycloalkylalkyl, C₆-C₁₄ cycloalkylcycloalkyl, C₁-C₇ alkoxy, C₁-C₇ haloalkoxy, C₂-C₇ alkoxyalkyl, C₁-C₇ alkylthio, C₁-C₇ haloalkylthio, C₂-C₇ alkylthioalkyl, C₁-C₇ alkylsulfinyl, C₁-C₇ alkylsulfonyl, C₁-C₇ haloalkylsulfinyl, C₁-C₇ haloalkylsulfonyl, C₁-C₇ alkylamino, C₂-C₇ dialkylamino, C₁-C₇ hydroxyalkyl, —SCN or CH═NOR¹¹; or C₁-C₇ alkyl or C₁-C₇ haloalkyl, each optionally substituted with up to 3 substituents independently selected from hydroxy, cyano, C(═O)OR⁸, C(═O)NR^{9a}R^{9b}, C(═O)R¹⁰ and CH═NOR¹¹;
each R^{5a}, R^{5b} and R^{5c} is independently halogen, cyano, hydroxy, nitro, C₁-C₇ alkyl, C₂-C₇ alkenyl, C₂-C₇ alkynyl, C₁-C₇ haloalkyl, C₂-C₇ haloalkenyl, C₃-C₇ cycloalkyl, C₃-C₇ halocycloalkyl, C₄-C₁₀ alkylcycloalkyl, C₄-C₁₀ cycloalkylalkyl, C₆-C₁₄ cycloalkylcycloalkyl, C₃-C₇ cycloalkoxy, C₃-C₇ halocycloalkoxy, C₁-C₇ alkoxy, C₁-C₇ haloalkoxy, C₁-C₆ alkylthio, C₁-C₇ haloalkylthio, C₁-C₇ alkylsulfinyl, C₁-C₇ alkylsulfonyl, C₁-C₇ haloalkylsulfinyl, C₁-C₇ haloalkylsulfonyl, C₁-C₇ alkylamino, C₂-C₇ dialkylamino, C₂-C₇ alkylcarbonyl, C₂-C₇ alkoxycarbonyl, C₂-C₇ alkylcarbonylamino, C₃-C₁₀ trialkylsilyl, SF₅, —SCN, C(═S)NH₂ or -U-V-T;
each U is independently O, S(═O)ₙ, NR¹² or a direct bond;
each V is independently C₁-C₆ alkylene, C₂-C₆ alkenylene, C₃-C₆ alkynylene, C₃-C₆ cycloalkylene or C₃-C₆ cycloalkenylene, wherein up to 3 carbon atoms are independently selected from C(═O), each optionally substituted with up to 5 substituents independently selected from halogen, cyano, nitro, hydroxy, C₁-C₆ alkyl, C₁-C₆ haloalky, C₁-C₆ alkoxy and C₁-C₆ haloalkoxy;
each T is independently NR^{13a}R^{13b}, OR¹⁴ or S(═O)ₙR¹⁴;
each R^{7a} is independently H, cyano or C₁-C₄ alkyl;
each R^{7b} is independently H or C₁-C₄ alkyl; or
a pair of R^{7a} and R^{7b} attached to the same carbon atom are taken together with the carbon atom to form a 3- to 6-membered saturated carbocyclic ring;
each R⁶ is independently H, cyano, C₁-C₃ alkyl or C₁-C₃ haloalkyl;
each R⁸, R^{9a}, R^{9b}, R¹⁰ and R¹¹ is independently H, C₁-C₇ alkyl, C₂-C₇ alkenyl, C₂-C₇ alkynyl, C₁-C₇ haloalkyl, C₂-C₇ haloalkenyl, C₃-C₇ cycloalkyl or C₃-C₇ halocycloalkyl;
each R¹² is independently H, C₁-C₆ alkyl, C₁-C₆ haloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R^{13a} and R^{13b} is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl); or
a pair of R^{13a} and R^{13b} attached to the same nitrogen atom are taken together with the nitrogen atom to form a 3- to 6-membered heterocyclic ring, the ring optionally substituted with up to 5 substituents independently selected from R¹⁵;
each R¹⁴ is independently H, C₁-C₆ alkyl, C₁-C₆ haloalkyl, C₂-C₆ alkenyl, C₃-C₆ alkynyl, C₃-C₆ cycloalkyl, C₃-C₆ halocycloalkyl, C₂-C₆ alkylcarbonyl, C₂-C₆ alkoxycarbonyl, C₂-C₆ (alkylthio)carbonyl, C₂-C₆ alkoxy(thiocarbonyl), C₄-C₈ cycloalkylcarbonyl, C₄-C₈ cycloalkoxycarbonyl, C₄-C₈ (cycloalkylthio)carbonyl or C₄-C₈ cycloalkoxy(thiocarbonyl);
each R¹⁵ is independently halogen, C₁-C₆ alkyl, C₁-C₆ haloalkyl or C₁-C₆ alkoxy;
each n is independently 0, 1 or 2; and
each p and f are independently 0, 1 or 2 in each instance of S(═O)ₚ(═NR⁶)_{f}, provided that the sum of p and f is 0, 1 or 2;
provided that:
(a) when J is R¹, then X is CQ³;
(b) when J is Q², then X is N or CR², and if X is N or CH, then Z is other than CH;
(c) for compounds other than 1-(4-chlorophenyl)-5-(4-fluorophenyl)-2-methyl-1H-imidazole or 4-chloro-1-(4-chlorophenyl)-5-(4-fluorophenyl)-2-methyl-1H-imidazole, when Q¹ is a phenyl ring which is unsubstituted by R^{5a} at both ortho positions, then when X is N or CR² and Q² is a phenyl ring, the Q² phenyl ring is substituted by at least one R^{5b} at an ortho position; and when X is CQ³ and Q³ is a phenyl ring, the Q³ phenyl ring is substituted by at least one R^{5c} at an ortho position;
(d) at least one and no more than two of X, Y and Z is nitrogen;
(e) the compound is not a compound of formula F-1 through F-4, as shown below 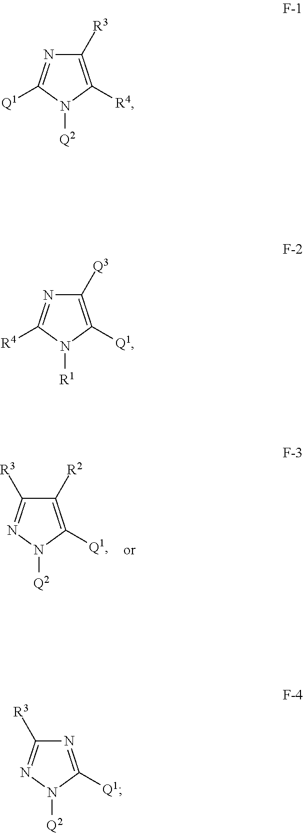
(g) the compound is not 4-[2-ethyl-1-(4-methoxyphenyl)-1H-imidazol-5-yl]-pyridine, 4-[1-(4-methoxyphenyl)-2-methyl-1H-imidazol-5-yl]pyridine or 3,5-dichloro-2-(4-iodo-5-phenyl-1H-1,2,3-triazol-1-yl)pyridine; and
(e) when J is Q², X is CR², Y is N and Z is N, then R² is other than H.

2. A compound of claim 1 wherein:
Q¹ is a phenyl ring or a naphthalenyl ring system, each ring or ring system optionally substituted with up to 3 substituents independently selected from R^{5a}; or a 5- to 6-membered fully unsaturated heterocyclic ring containing ring members selected from carbon atoms and up to 3 heteroatoms independently selected from up to 2O, up to 2S and up to 3N atoms, wherein up to 2 carbon atom ring members are independently selected from C(═O) and C(═S), and the sulfur atom ring members are independently selected from S(═O)ₛ(═NR⁶)_{f}, the ring optionally substituted with up to 3 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W¹;
W¹ is a phenyl, thienyl, pyridinyl, pyridazinyl, pyrimidinyl or pyrazolyl ring optionally substituted with up to 3 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members;
Q² is a phenyl ring or a naphthalenyl ring system, each ring or ring system optionally substituted with up to 3 substituents independently selected from R^{5b}; or a 5- to 6-membered fully unsaturated heterocyclic ring containing ring members selected from carbon atoms and up to 3 heteroatoms independently selected from up to 2O, up to 2S and up to 3N atoms, wherein up to 2 carbon atom ring members are independently selected from C(═O) and C(═S), and the sulfur atom ring members are independently selected from S(═O)ₛ(═NR⁶)_{f}, the ring optionally substituted with up to 3 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W²;
W² is a phenyl, thienyl, pyridinyl, pyridazinyl, pyrimidinyl or pyrazolyl ring optionally substituted with up to 3 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members;
Q³ is a phenyl ring or a naphthalenyl ring system, each ring or ring system optionally substituted with up to 3 substituents independently selected from R^{5c}; or a 5- to 6-membered fully unsaturated heterocyclic ring containing ring members selected from carbon atoms and up to 3 heteroatoms independently selected from up to 2O, up to 2S and up to 3N atoms, wherein up to 2 carbon atom ring members are independently selected from C(═O) and C(═S), and the sulfur atom ring members are independently selected from S(═O)ₛ(═NR⁶)_{f}, the ring optionally substituted with up to 3 substituents independently selected from R^{5c} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W³;
W³ is a phenyl, thienyl, pyridinyl, pyridazinyl, pyrimidinyl or pyrazolyl ring optionally substituted with up to 3 substituents independently selected from R^{5c} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members;
each R^{7a} is independently H, cyano or methyl;
each R^{7b} is independently H or methyl; or
a pair of R^{7a} and R^{7b} attached to the same carbon atom are taken together with the carbon atom to form a cyclopropyl ring.

3. A compound of claim 2 wherein:
Q¹ is a phenyl, thienyl, pyridinyl, pyridazinyl, pyrimidinyl or pyrazolyl ring or a naphthalenyl ring system, each ring or ring system optionally substituted with up to 3 substituents independently selected from R^{5a} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W¹;
W¹ is a phenyl or pyridinyl ring optionally substituted with up to 3 substituents independently selected from R^{5a};
Q² is a phenyl, thienyl, pyridinyl, pyridazinyl, pyrimidinyl or pyrazolyl ring or a naphthalenyl ring system, each ring or ring system optionally substituted with up to 3 substituents independently selected from R^{5b} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W²;
W² is a phenyl or pyridinyl ring optionally substituted with up to 3 substituents independently selected from R^{5b};
Q³ is a phenyl, thienyl, pyridinyl, pyridazinyl, pyrimidinyl or pyrazolyl ring or a naphthalenyl ring system, each ring or ring system optionally substituted with up to 3 substituents independently selected from R^{5c} on carbon atom ring members and selected from cyano, C₁-C₆ alkyl, C₂-C₆ alkenyl, C₂-C₆ alkynyl, C₃-C₆ cycloalkyl, C₁-C₆ alkoxy, C₂-C₆ alkoxyalkyl, C₂-C₆ alkylcarbonyl, C₂-C₆ alkoxycarbonyl, C₂-C₆ alkylaminoalkyl and C₃-C₆ dialkylaminoalkyl on nitrogen atom ring members; or C(R^{7a}R^{7b})W³;
W³ is a phenyl or pyridinyl ring optionally substituted with up to 3 substituents independently selected from R^{5c};
each R², R³ and R⁴ is independently H, halogen, C₂-C₃ alkenyl, C₂-C₃ alkynyl, C₂-C₃ haloalkenyl, C₃-C₆ cycloalkyl, C₁-C₃ alkoxy, C₁-C₃ alkylthio, C₁-C₃ alkylamino, C₂-C₄ dialkylamino or C₁-C₃ hydroxyalkyl; or C₁-C₃ alkyl or C₁-C₃ haloalkyl, each optionally substituted with up to 1 substituent independently selected from hydroxy, cyano, C(═O)OR⁸, C(═O)NR^{9a}R^{9b}, C(═O)R¹⁰ and CH═NOR¹¹;
each R^{5a}, R^{5b} and R^{5c} is independently halogen, cyano, C₁-C₃ alkyl, C₂-C₃ alkenyl, C₂-C₃ alkynyl, C₁-C₃ haloalkyl, C₃ cycloalkyl, C₁-C₃ alkoxy, C₁-C₃ haloalkoxy, C₁-C₃ alkylthio, C₁-C₃ alkylamino, C₂-C₄ dialkylamino C₂-C₄ alkylcarbonyl, C₂-C₄ alkoxycarbonyl, C₂-C₄ alkylcarbonylamino or -U-V-T;
U is O or NH;
V is C₂-C₄ alkylene;
T is NR^{13a}R^{13b} or OR¹⁴;
each R^{7a} and R^{7b} is independently H or methyl;
each R⁸, R^{9a}, R^{9b}, R¹⁰ and R¹¹ is independently H or methyl;
each R^{13a} and R^{13b} is independently H, C₁-C₆ alkyl or C₁-C₆ haloalkyl; and
each R¹⁴ is independently H, C₁-C₆ alkyl or C₁-C₆ haloalkyl.

4. A compound of claim 3 wherein:
Q¹ is a phenyl or pyridinyl ring optionally substituted with up to 3 substituents independently selected from R^{5a};
Q² is a phenyl or pyridinyl ring optionally substituted with up to 3 substituents independently selected from R^{5b}; and
Q³ is a phenyl or pyridinyl ring optionally substituted with up to 3 substituents independently selected from R^{5c}.

5. A compound of claim 4 wherein:
each R², R³ and R⁴ is independently H, halogen, cyano or C₁-C₃ alkyl; and
each R^{5a}, R^{5b} and R^{5c} is independently halogen, cyano, C₁-C₃ alkyl, C₂-C₃ alkenyl, C₁-C₃ haloalkyl, C₁-C₃ alkoxy, C₁-C₃ alkylthio or C₁-C₃ alkylamino.

6. A compound of claim 5 wherein:
J is Q²;
X is CR²;
Y is N;
Z is CR⁴;
each R² and R⁴ is independently Cl, Br, I or C₁-C₂ alkyl;
each R^{5a} and R^{5b} is independently F, Cl, Br, cyano, C₁-C₂ alkyl, C₁-C₂ haloalkyl or C₁-C₂ alkoxy; and
one of the Q¹ and Q² rings is substituted with 2 to 3 substituents and the other of the Q¹ and Q² rings is substituted with 1 to 2 substituents.

7. A compound of claim 1 which is selected from the group consisting of:
4-chloro-1-(4-chlorophenyl)-5-(2,4,6-trifluorophenyl)-1H-imidazole;
4-chloro-1-(4-chlorophenyl)-5-(2,6-difluorophenyl)-2-methyl-1H-imidazole;
2,4-dichloro-1-(4-chlorophenyl)-5-(2,6-difluorophenyl)-1H-imidazole;
1-(4-chlorophenyl)-5-(2,6-difluorophenyl)-2,4-dimethyl-1H-imidazole;
4-chloro-1-(4-chlorophenyl)-2-methyl-5-(2,4,6-trifluorophenyl)-1H-imidazole;
1-(4-chlorophenyl)-2,4-dimethyl-5-(2,4,6-trifluorophenyl)-1H-imidazole;
1-(4-chlorophenyl)-2,4-dichloro-5-(2,4,6-trifluorophenyl)-1H-imidazole;
1-(4-chlorophenyl)-5-(2,6-difluoro-4-methoxyphenyl)-2,4-dimethyl-1H-imidazole;
1-(4-chlorophenyl)-2,4-dichloro-5-(2,6-difluoro-4-methoxyphenyl)-1H-imidazole;
4-chloro-1-(4-chlorophenyl)-5-(2,6-difluoro-4-methoxyphenyl)-2-methyl-1H-imidazole;
4-chloro-1-(4-chloro-3-fluorophenyl)-2-methyl-5-(2,4,6-trifluorophenyl)-1H-imidazole;
1-(4-chloro-3-fluorophenyl)-2,4-dichloro-5-(2,4,6-trifluorophenyl)-1H-imidazole;
1-(4-chloro-3-fluorophenyl)-2,4-dimethyl-5-(2,4,6-trifluorophenyl)-1H-imidazole;
1-(4-chloro-3-fluorophenyl)-5-(2,6-difluorophenyl)-2,4-dimethyl-1H-imidazole;
1-(4-chloro-3-fluorophenyl)-2,4-dichloro-5-(2,6-difluorophenyl)-1H-imidazole;
4-chloro-1-(4-chloro-3-fluorophenyl)-5-(2,6-difluoro-4-methoxyphenyl)-2-methyl-1H-imidazole;
1-(4-chloro-3-fluorophenyl)-5-(2,6-difluoro-4-methoxyphenyl)-2,4-dimethyl-1H-imidazole;
1-(4-chloro-3-fluorophenyl)-2,4-dichloro-5-(2,6-difluoro-4-methoxyphenyl)-1H-imidazole;
4-chloro-1-(3-fluorophenyl)-2-methyl-5-(2,4,6-trifluorophenyl)-1H-imidazole;
2,4-dichloro-1-(3-fluorophenyl)-5-(2,4,6-trifluorophenyl)-1H-imidazole;
4-chloro-1-(3-fluorophenyl)-5-(2,6-difluoro-4-methoxyphenyl)-2-methyl-1H-imidazole;
1-(3-fluorophenyl)-5-(2,6-difluoro-4-methoxyphenyl)-2,4-dimethyl-1H-imidazole;
1-(3-fluorophenyl)-2,4-dichloro-5-(2,6-difluoro-4-methoxyphenyl)-1H-imidazole;
4-chloro-1-(4-chlorophenyl)-2-methyl-5-(2,3,6-trifluorophenyl)-1H-imidazole;
1-(4-chlorophenyl)-2,4-dimethyl-5-(2,3,6-trifluorophenyl)-1H-imidazole;
1-(4-chlorophenyl)-2,4-dichloro-5-(2,3,6-trifluorophenyl)-1H-imidazole;
4-chloro-1-(3-fluorophenyl)-2-methyl-5-(2,3,6-trifluorophenyl)-1H-imidazole;
1-(3-fluorophenyl)-2,4-dimethyl-5-(2,3,6-trifluorophenyl)-1H-imidazole;
1-(3-fluorophenyl)-2,4-dichloro-5-(2,3,6-trifluorophenyl)-1H-imidazole;
4-chloro-5-(2-chloro-4-fluorophenyl)-1-(4-chlorophenyl)-2-methyl-1H-imidazole;
5-(2-chloro-4-fluorophenyl)-1-(4-chlorophenyl)-2,4-dimethyl-1H-imidazole;
5-(2-chloro-4-fluorophenyl)-1-(4-chlorophenyl)-2,4-dichloro-1H-imidazole;
4-chloro-1-(4-chlorophenyl)-5-(2,6-difluoro-3-methoxyphenyl)-2-methyl-1H-imidazole;
4-chloro-5-(2,6-difluoro-4-methoxyphenyl)-1-(4-fluorophenyl)-2-methyl-1H-imidazole;
4-chloro-5-(2,6-difluoro-4-methoxyphenyl)-2-methyl-1-(4-methylphenyl)-1H-imidazole;
4-[4-chloro-1-(4-chlorophenyl)-2-methyl-1H-imidazol-5-yl]-3,5-difluorobenzonitrile;
2-bromo-4-chloro-5-(2,6-difluoro-4-methoxyphenyl)-1-(3-fluorophenyl)-1H-imidazole;
2-chloro-1-(4-chloro-3-fluorophenyl)-5-(2,6-difluoro-4-methoxyphenyl)-4-methyl-1H-imidazole;
2-chloro-5-[2-chloro-5-(2,6-difluoro-4-methoxyphenyl)-4-methyl-1H-imidazol-1-yl]pyridine;
2-bromo-4-chloro-5-(2,6-difluoro-3-methoxyphenyl)-1-(4-fluorophenyl)-1H-imidazole;
4-chloro-5-(2,6-difluoro-3-methoxyphenyl)-2-methyl-1-(4-methylphenyl)-1H-imidazole;
4-chloro-5-(2,6-difluoro-3-methoxyphenyl)-1-(4-fluorophenyl)-2-methyl-1H-imidazole;
2,4-dichloro-5-(2,6-difluoro-3-methoxyphenyl)-1-(4-fluorophenyl)-1H-imidazole;
2,4-dichloro-1-(4-chloro-3-fluorophenyl)-5-(2,6-difluoro-3-methoxyphenyl)-1H-imidazole;
4-chloro-1-(4-chloro-3-fluorophenyl)-5-(2,6-difluoro-3-methoxyphenyl)-2-methyl-1H-imidazole;
2-chloro-5-[2,4-dichloro-5-(2,6-difluoro-4-methoxyphenyl)-1H-imidazol-1-yl]pyridine;
4-chloro-1-[3-(difluoromethoxy)phenyl]-5-(2,6-difluoro-3-methoxyphenyl)-2-methyl-1H-imidazole;
3-[4-chloro-1-(4-chlorophenyl)-2-methyl-1H-imidazol-5-yl]-2,4-difluorobenzonitrile;
4-chloro-5-(2,6-difluoro-4-methoxyphenyl)-1-(3,4-difluorophenyl)-2-methyl-1H-imidazole;
4-chloro-1-(3-chlorophenyl)-5-(2,6-difluoro-4-methoxyphenyl)-2-methyl-1H-imidazole;
4-bromo-2-chloro-1-(4-chlorophenyl)-5-(2,6-difluoro-3-methoxyphenyl)-1H-imidazole;
5-[2,4-dichloro-5-(2,6-difluoro-4-methoxyphenyl)-1H-imidazol-yl]-2-methylpyridine;
5-[2,4-dibromo-5-(2,6-difluoro-4-methoxyphenyl)-1H-imidazol-1-yl]-2-methylpyridine;
2-chloro-5-(4-chlorophenyl)-1-(2,6-difluoro-4-methoxyphenyl)-4-methyl-imidazole;
4-chloro-5-(2,6-difluoro-4-methoxyphenyl)-1-(4-fluorophenyl)-1H-imidazole-2-carboxaldehyde oxime;
4-chloro-1-(2,6-dichlorophenyl)-2-methyl-5-[(2,4,6-trifluorophenyl)methyl]-1H-imidazole;
2-chloro-5-[2,4-dichloro-5-(2,6-difluoro-4-methoxyphenyl)-1H-imidazol-1-yl-4-methyl]pyridine;
4-(2-chloro-4-fluorophenyl)-5-[(2,4-difluorophenyl)methyl]-1,3-dimethyl-1H-pyrazole; and
2-chloro-1-(4-chlorophenyl)-5-(2,6-difluorophenyl)-4-methyl-1H-imidazole;
2-chloro-1-(4-chlorophenyl)-4-methyl-5-(2,4,6-trifluorophenyl)-1H-imidazole;
2-chloro-1-(4-chlorophenyl)-5-(2,6-difluoro-4-methoxyphenyl)-4-methyl-1H-imidazole;
2-chloro-1-(4-chloro-3-fluorophenyl)-4-methyl-5-(2,4,6-trifluorophenyl)-1H-imidazole;
2-chloro-1-(4-chloro-3-fluorophenyl)-5-(2,6-difluoro-4-methoxyphenyl)-4-methyl-1H-imidazole;
2-chloro-1-(3-fluorophenyl)-4-methyl-5-(2,4,6-trifluorophenyl)-1H-imidazole;
2-chloro-1-(3-fluorophenyl)-5-(2,6-difluoro-4-methoxyphenyl)-4-methyl-1H-imidazole;
2-chloro-1-(4-chlorophenyl)-4-methyl-5-(2,3,6-trifluorophenyl)-1H-imidazole;
2-chloro-1-(3-fluorophenyl)-4-methyl-5-(2,3,6-trifluorophenyl)-1H-imidazole;
2-chloro-1-(4-chlorophenyl)-5-(2,6-difluoro-3-methyoxyphenyl)-4-methyl-1H-imidazole;
2-chloro-5-(2,6-difluoro-4-methoxyphenyl)-1-(4-fluorolphenyl)-4-methyl-1H-imidazole;
2-chloro-5-(2,6-difluoro-4-methoxyphenyl)-4-methyl-1-(4-methylphenyl)-1H-imidazole;
4-[2-chloro-1-(4-chlorophenyl)-4-methyl-1H-imidazol-5-yl]-3,5-difluorobenzonitrile;
2-chloro-5-(2,6-difluoro-3-methoxyphenyl)-4-methyl-1-(4-methylphenyl)-1H-imidazole;
2-chloro-5-(2,6-difluoro-3-methoxyphenyl)-1-(4-fluorophenyl)-4-methyl-1H-imidazole;
2-chloro-1-(4-chloro-3-fluorophenyl)-5-(2,6-difluoro-3-methoxyphenyl)-1H-imidazole;
2-chloro-1-[3-(difluoromethoxy)phenyl]-5-(2,6-difluoro-3-methoxyphenyl)-4-methyl-1H-imidazole;
3-[2-chloro-1-(4-chlorophenyl)-4-methyl-1H-imidazol-5-yl]-2,4-difluorobenzonitrile;
2-chloro-5-(2,6-difluoro-4-methoxyphenyl)-1-(3,4-difluorophenyl)-4-methyl-1H-imidazole;
2-chloro-1-(3-chlorophenyl)-5-(2,6-difluoro-4-methoxyphenyl)-4-methyl-1H-imidazole;
2-fluoro-5-[2-chloro-5-(2,6-difluoro-4-methoxyphenyl)-4-methyl-1H-imidazol-1-yl]pyridine;
2-bromo-5-[2-chloro-5-(2,6-difluoro-4-methoxyphenyl)-4-methyl-1H-imidazol-1-yl]pyridine;
2-methoxy-5-[2-chloro-5-(2,6-difluoro-4-methoxyphenyl)-4-methyl-1H-imidazol-1-yl]pyridine;
2-trifluoromethoxy-5-[2-chloro-5-(2,6-difluoro-4-methoxyphenyl)-4-methyl-1H-imidazol-1-yl]pyridine;
2-trifluoromethyl-5-[2-chloro-5-(2,6-difluoro-4-methoxyphenyl)-4-methyl-1H-imidazol-1-yl]pyridine;
2-cyano-5-[2-chloro-5-(2,6-difluoro-4-methoxyphenyl)-4-methyl-1H-imidazol-1-yl]pyridine;
3-fluoro-5-[2-chloro-5-(2,6-difluoro-4-methoxyphenyl)-4-methyl-1H-imidazol-1-yl]pyridine;
3-bromo-5-[2-chloro-5-(2,6-difluoro-4-methoxyphenyl)-4-methyl-1H-imidazol-1-yl]pyridine;
3-chloro-5-[2-chloro-5-(2,6-difluoro-4-methoxyphenyl)-4-methyl-1H-imidazol-1-yl]pyridine;
3-methoxy-5-[2-chloro-5-(2,6-difluoro-4-methoxyphenyl)-4-methyl-1H-imidazol-1-yl]pyridine;
1-(4-chlorophenyl)-5-(2,6-difluoro-4-ethoxyphenyl)-2,4-dimethyl-1H-imidazole;
1-(4-fluorophenyl)-5-(2,6-difluoro-4-ethoxyphenyl)-2,4-dimethyl-1H-imidazole;
1-(3-chlorophenyl)-5-(2,6-difluoro-4-ethoxyphenyl)-2,4-dimethyl-1H-imidazole;
1-(3-fluorophenyl)-5-(2,6-difluoro-4-ethoxyphenyl)-2,4-dimethyl-1H-imidazole;
1-(4-methylphenyl)-5-(2,6-difluoro-4-ethoxyphenyl)-2,4-dimethyl-1H-imidazole; and
1-(3-methylphenyl)-5-(2,6-difluoro-4-ethoxyphenyl)-2,4-dimethyl-1H-imidazole.

8. A compound of claim 1 which is selected from the group consisting of:
4-chloro-1-(4-chlorophenyl)-5-(2,4,6-trifluorophenyl)-1H-imidazole;
4-chloro-1-(4-chlorophenyl)-5-(2,6-difluorophenyl)-2-methyl-1H-imidazole;
2,4-dichloro-1-(4-chlorophenyl)-5-(2,6-difluorophenyl)-1H-imidazole;
1-(4-chlorophenyl)-2,4-dichloro-5-(2,4,6-trifluorophenyl)-1H-imidazole;
1-(4-chlorophenyl)-5-(2,6-difluorophenyl)-2,4-dimethyl-1H-imidazole;
4-chloro-1-(4-chlorophenyl)-2-methyl-5-(2,4,6-trifluorophenyl)-1H-imidazole;
1-(4-chlorophenyl)-2,4-dimethyl-5-(2,4,6-trifluorophenyl)-1H-imidazole;
1-(4-chlorophenyl)-2,4-dichloro-5-(2,4,6-trifluorophenyl)-1H-imidazole;
1-(4-chlorophenyl)-5-(2,6-difluoro-4-methoxyphenyl)-2,4-dimethyl-1H-imidazole;
1-(4-chlorophenyl)-2,4-dichloro-5-(2,6-difluoro-4-methoxyphenyl)-1H-imidazole;
4-chloro-1-(4-chlorophenyl)-5-(2,6-difluoro-4-methoxyphenyl)-2-methyl-1H-imidazole;
4-chloro-1-(4-chloro-3-fluorophenyl)-2-methyl-5-(2,4,6-trifluorophenyl)-1H-imidazole;
1-(4-chloro-3-fluorophenyl)-2,4-dichloro-5-(2,4,6-trifluorophenyl)-1H-imidazole;
1-(4-chloro-3-fluorophenyl)-2,4-dimethyl-5-(2,4,6-trifluorophenyl)-1H-imidazole;
1-(4-chloro-3-fluorophenyl)-5-(2,6-difluorophenyl)-2,4-dimethyl-1H-imidazole;
1-(4-chloro-3-fluorophenyl)-2,4-dichloro-5-(2,6-difluorophenyl)-1H-imidazole;
1-(4-chloro-3-fluorophenyl)-5-(2,6-difluorophenyl)-2,4-dimethyl-1H-imidazole;
4-chloro-1-(4-chloro-3-fluorophenyl)-5-(2,6-difluoro-4-methoxyphenyl)-2-methyl-1H-imidazole;
1-(4-chloro-3-fluorophenyl)-5-(2,6-difluoro-4-methoxyphenyl)-2,4-dimethyl-1H-imidazole;
1-(4-chloro-3-fluorophenyl)-2,4-dichloro-5-(2,6-difluoro-4-methoxyphenyl)-1H-imidazole;
4-chloro-1-(3-fluorophenyl)-2-methyl-5-(2,4,6-trifluorophenyl)-1H-imidazole;
2,4-dichloro-1-(3-fluorophenyl)-5-(2,4,6-trifluorophenyl)-1H-imidazole;
1-(3-fluorophenyl)-5-(2,4,6-trifluorophenyl)-2,4-dimethyl-1H-imidazole;
4-chloro-1-(3-fluorophenyl)-5-(2,6-difluoro-4-methoxyphenyl)-2-methyl-1H-imidazole;
1-(4-fluorophenyl)-5-(2,6-difluoro-4-methoxyphenyl)-2,4-dimethyl-1H-imidazole;
1-(3-fluorophenyl)-2,4-dichloro-5-(2,6-difluoro-4-methoxyphenyl)-1H-imidazole;
1-(3-fluorophenyl)-2,4-dichloro-5-(2,6-difluoro-4-methoxyphenyl)-1H-imidazole;
1-(3-fluorophenyl)-2,4-dimethyl-5-(2,6-difluoro-4-methoxyphenyl)-1H-imidazole;
4-chloro-1-(3-fluorophenyl)-5-(2,6-difluoro-4-methoxyphenyl)-2-methyl-1H-imidazole;
4-chloro-1-(4-chlorophenyl)-2-methyl-5-(2,3,6-trifluorophenyl)-1H-imidazole;
1-(4-chlorophenyl)-2,4-dimethyl-5-(2,3,6-trifluorophenyl)-1H-imidazole; and
1-(4-chlorophenyl)-2,4-dichloro-5-(2,3,6-trifluorophenyl)-1H-imidazole;
4-chloro-1-(3-fluorophenyl)-2-methyl-5-(2,3,6-trifluorophenyl)-1H-imidazole;
1-(3-fluorophenyl)-2,4-dimethyl-5-(2,3,6-trifluorophenyl)-1H-imidazole;
1-(3-fluorophenyl)-2,4-dichloro-5-(2,3,6-trifluorophenyl)-1H-imidazole;
4-chloro-5-(2-chloro-4-fluorophenyl)-1-(4-chlorophenyl)-2-methyl-1H-imidazole;
5-(2-chloro-4-fluorophenyl)-1-(4-chlorophenyl)-2,4-dimethyl-1H-imidazole; and
5-(2-chloro-4-fluorophenyl)-1-(4-chlorophenyl)-2,4-dichloro-1H-imidazole.

9. A fungicidal composition comprising (a) a compound of claim 1; and (b) at least one other fungicide.

10. A fungicidal composition comprising (a) a fungicidally effective amount of a compound of claim 1; and (b) at least one additional component selected from the group consisting of surfactants, solid diluents and liquid diluents.

11. A method for controlling plant diseases caused by fungal plant pathogens comprising applying to the plant or portion thereof, or to the plant seed, a fungicidally effective amount of a compound of claim 1.
